(19) United States
(12) Patent Application Publication
(10) Pub. No.: US 2023/0117680 A1
(43) Pub. Date: Apr. 20, 2023

(54) CYCLOPHILIN D INHIBITORS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Alexander A. Peterson, Cambridge, MA (US); Alvin Hsu, Cambridge, MA (US)

(73) Assignees: The Broad Institute, Inc., Cambridge, MA (US); President and Fellows of Harvard College, Cambridge, MA (US)

(21) Appl. No.: 17/784,075

(22) PCT Filed: Dec. 10, 2020
(86) PCT No.: PCT/US2020/064200

§ 371 Date: Jun. 9, 2022

Related U.S. Application Data
(60) Provisional application No. 62/946,320, filed on Dec. 10, 2019.

(51) Int. Cl. C07K 7/56

(52) U.S. Cl. CPC C07K 7/56

(57) ABSTRACT

Provided herein are compounds of Formula (I′) or (I), and pharmaceutically acceptable salts, solvates, hydrates, polymorphs, co-crystals, tautomers, stereoisomers, isotopically labeled derivatives, prodrugs, and compositions thereof. Also provided are methods and kits involving the inventive compounds or compositions for treating and/or preventing diseases and/or conditions (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins) in a subject, as well as for reducing oxidative stress. Provided are methods of inhibiting a cyclophilin (e.g., CypB, CypC, CypD, CypE, CypG, CypH, Cyp40, PPWD1, PPIL1, NKTR) in a subject and/or biological sample.

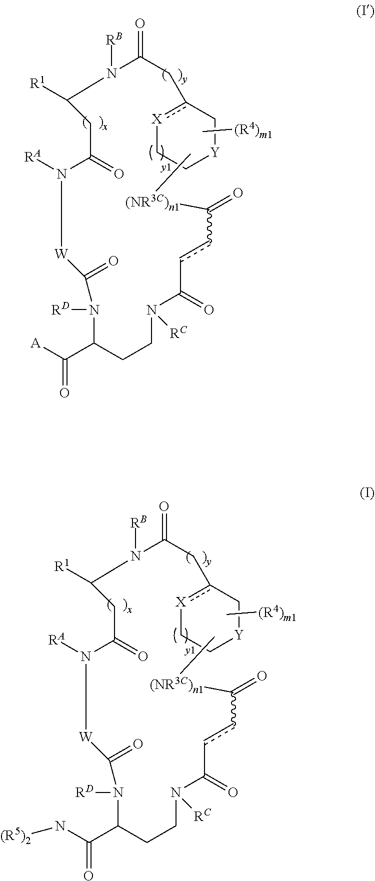

### RELATED APPLICATIONS

This application claims priority under 35 U.S.C. § 119(e) to U.S. Provisional Application Ser. No. 62/946,320, filed Dec. 10, 2019, which is incorporated herein by reference.

### GOVERNMENT SUPPORT

This invention was made with government support under grant numbers R35GM118062 and R35GM119437 awarded by the National Institutes of Health. The government has certain rights in the invention.

### BACKGROUND OF THE INVENTION

The cyclophilin family is a group of 17 proteins characterized by a highly conserved peptidyl-prolyl-isomerase domain, with a majority of members possessing enzymatic activity to convert between cis and trans proline-peptide bonds. The mitochondrial isoform of this family, cyclophilin D (CypD), acts as a regulator of the mitochondrial permeability transition pore (mPTP), a channel across the inner mitochondrial membrane where prolonged opening results in cell necrosis. The mPTP has been implicated in diseases including ischemia/reperfusion injury and many neurodegenerative disorders. Several studies through knockout/knockdown and small molecule inhibition have linked CypD with amelioration of oxidatively induced cell death. Furthermore, CypD is the only consistently verified regulator of the mPTP, the structure and biochemical pathways of which still remain under study. Although this makes CypD as popular target for probing biology and new therapeutics, current inhibitors exhibit one of two types of promiscuous binding modes. The first, exemplified by the prototypical cyclophilin inhibitor Cyclosporine A (CsA), target a highly conserved active site. The second, which encompasses many de novo designed inhibitors, target both the active site and the adjacent S2 pocket, the latter of which is diversified across the cyclophilin family. However, the region of the S2 pocket that these inhibitors interact with is located at the base of the pocket, which displays only backbone moieties as opposed to sidechain functionalities. Both of these types of small molecule inhibitors promiscuously inhibit multiple cyclophilins, blurring the phenotypic data from biological mechanisms and offering undesired side effects as a therapeutic. However, there is a need to target inhibition of one specific cyclophilin over other cyclophilins. Accordingly, to both elucidate the biological roles and garner further therapeutic potential from cyclophilin (e.g., CypB, CypC, CypD, CypE, CypG, CypH, Cyp40, PPWD1, PPIL1, NKTR) inhibition, there is a need to identify new and specific inhibitors of cyclophilins (e.g., CypB, CypC, CypD, CypE, CypG, CypH, Cyp40, PPWD1, PPIL1, NKTR) and develop tools for studying inhibition of cyclophilins (e.g., CypB, CypC, CypD, CypE, CypG, CypH, Cyp40, PPWD1, PPIL1, NKTR), where the inhibitors of the cyclophilins (e.g., CypB, CypC, CypD, CypE, CypG, CypH, Cyp40, PPWD1, PPIL1, NKTR) can be used for treating a disease and/or condition (e.g., diseases associated with the regulation of the mitochondrial permeability transition pore (mPTP), autophagy, aging, neurodegenerative diseases, metabolic diseases, proliferative diseases, conditions associated with oxidative stress, mitochondrial diseases).

### SUMMARY OF THE INVENTION

To gain cyclophilin-specific inhibition over other cyclophilins, it has been hypothesized, but not limited to this hypothesis, that interacting with residues in the S2 pocket that have accessible sidechains could accomplish this. The most accessible and diverse of these residues are two “gatekeeper” residues on the opposite side of the S2 pocket (with respect to the active site). Accordingly described herein are cyclophilin inhibitors (e.g., selective cyclophilin inhibitors). Described herein are compounds of Formula (I′) or (I), and salts, solvates, hydrates, polymorphs, co-crystals, tautomers, stereoisomers, isotopically labeled derivatives, and prodrugs thereof, and mixtures thereof. The compounds of Formula (I′) or (I), and pharmaceutically acceptable salts, solvates, hydrates, polymorphs, co-crystals, tautomers, stereoisomers, isotopically labeled derivatives, prodrugs, and compositions thereof, may inhibit the activity of a cyclophilin (e.g., CypB, CypC, CypD, CypE, CypG, CypH, Cyp40, PPWD1, PPIL1, NKTR) in a biological sample or subject. In certain embodiments, a compound of Formula (I′) or (I) selectively inhibits one or more cyclophilins. In certain embodiments, the cyclophilin is CypD. In certain embodiments, the cyclophilin is cyclophilin A (CypA). In certain embodiments, the cyclophilin is cyclophilin B (CypB), cyclophilin C (CypC), cyclophilin E (CypE), cyclophilin G (CypG), cyclophilin H (CypH), or cyclophilin 40 (Cyp40), PPWD1, PPIL1, or NKTR. In certain embodiments, the compounds of Formula (I′) or (I) are selective for cyclophilin D compared to other cyclophilins (e.g., at least 2-fold, 5-fold, 10-fold, or more selective for cyclophilin D). Described herein are methods of using the inventive compounds, and pharmaceutically acceptable salts, solvates, hydrates, polymorphs, co-crystals, tautomers, stereoisomers, isotopically labeled derivatives, prodrugs, and compositions thereof, to study the inhibition of a cyclophilin (e.g., CypB, CypC, CypD, CypE, CypG, CypH, Cyp40, PPWD1, PPIL1, NKTR) or as therapeutics for the prevention and/or treatment of diseases associated with the overexpression and/or aberrant (e.g., increased or unwanted) activity of a cyclophilin (e.g., CypB, CypC, CypD, CypE, CypG, CypH, Cyp40, PPWD1, PPIL1, NKTR). The compounds described herein may be useful in treating and/or preventing a disease or condition, e.g., in treating and/or preventing a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, condition associated with modulating (e.g., regulating) the mPTP, cardiovascular condition (e.g., ischemia-reperfusion injury), stroke, heart attack, conditions associated with oxidative stress, mitochondrial diseases), in a subject in need thereof. Also provided are uses, pharmaceutical compositions, and kits including a compound described herein.

In one aspect, the present disclosure provides compounds of Formula (I′):

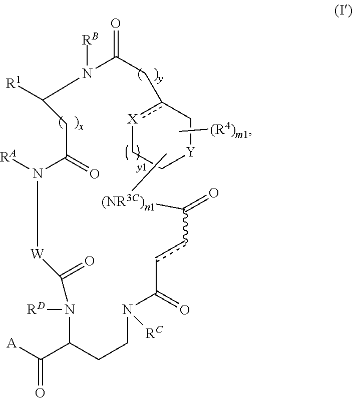

and pharmaceutically acceptable salts, solvates, hydrates, polymorphs, co-crystals, tautomers, stereoisomers, isotopically labeled derivatives, and prodrugs thereof, wherein R¹, R^{3C}, R⁴, R^{A}, R^{B}, R^{D}, x, y, y1, m1, n1, A, W, X, and Y are as defined herein. In certain embodiments, a compound of Formula (I′) is a compound of Formula (I). In certain embodiments, the compound of Formula (I′) or (I) is not a compound disclosed in PCT Application Publication No. WO 2019/168654, published Sep. 6, 2019. In certain embodiments, the compound of Formula (I′) or (I) is not a compound of formula:

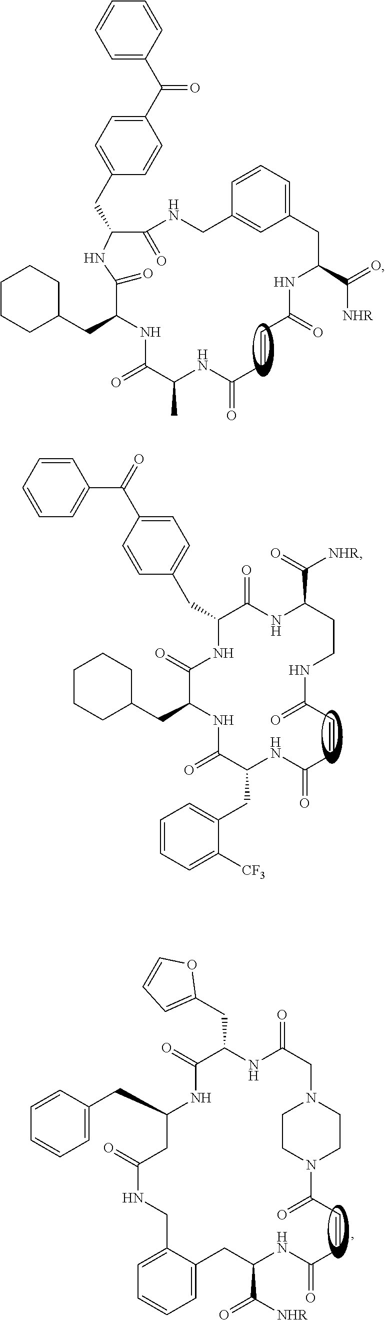

or pharmaceutically acceptable salts, solvates, hydrates, polymorphs, co-crystals, tautomers, stereoisomers, isotopically labeled derivatives, and prodrugs thereof. In certain embodiments, the compound of Formula (I′) or (I) is not a compound produced using DNA-templated synthesis and the building blocks:

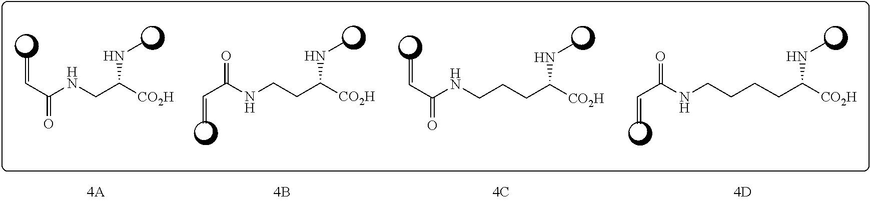

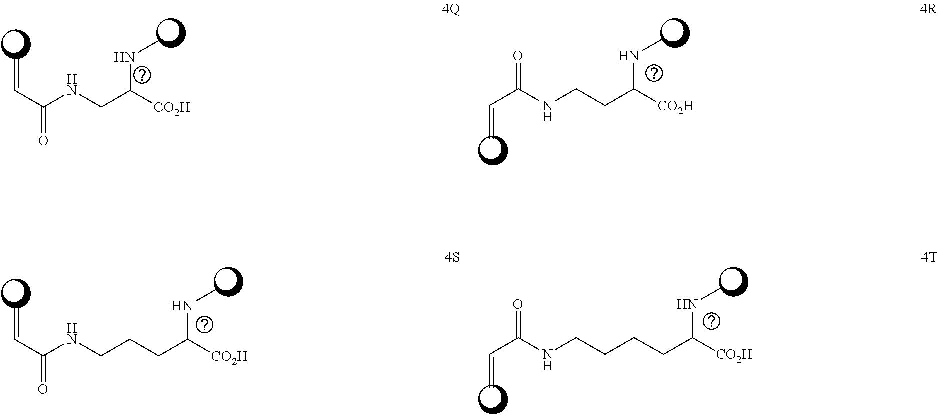

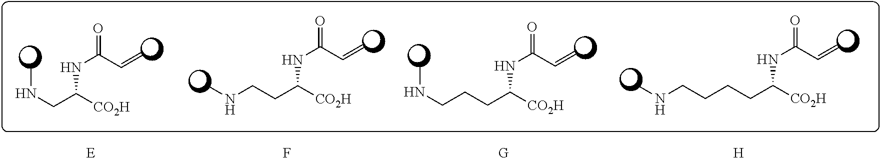

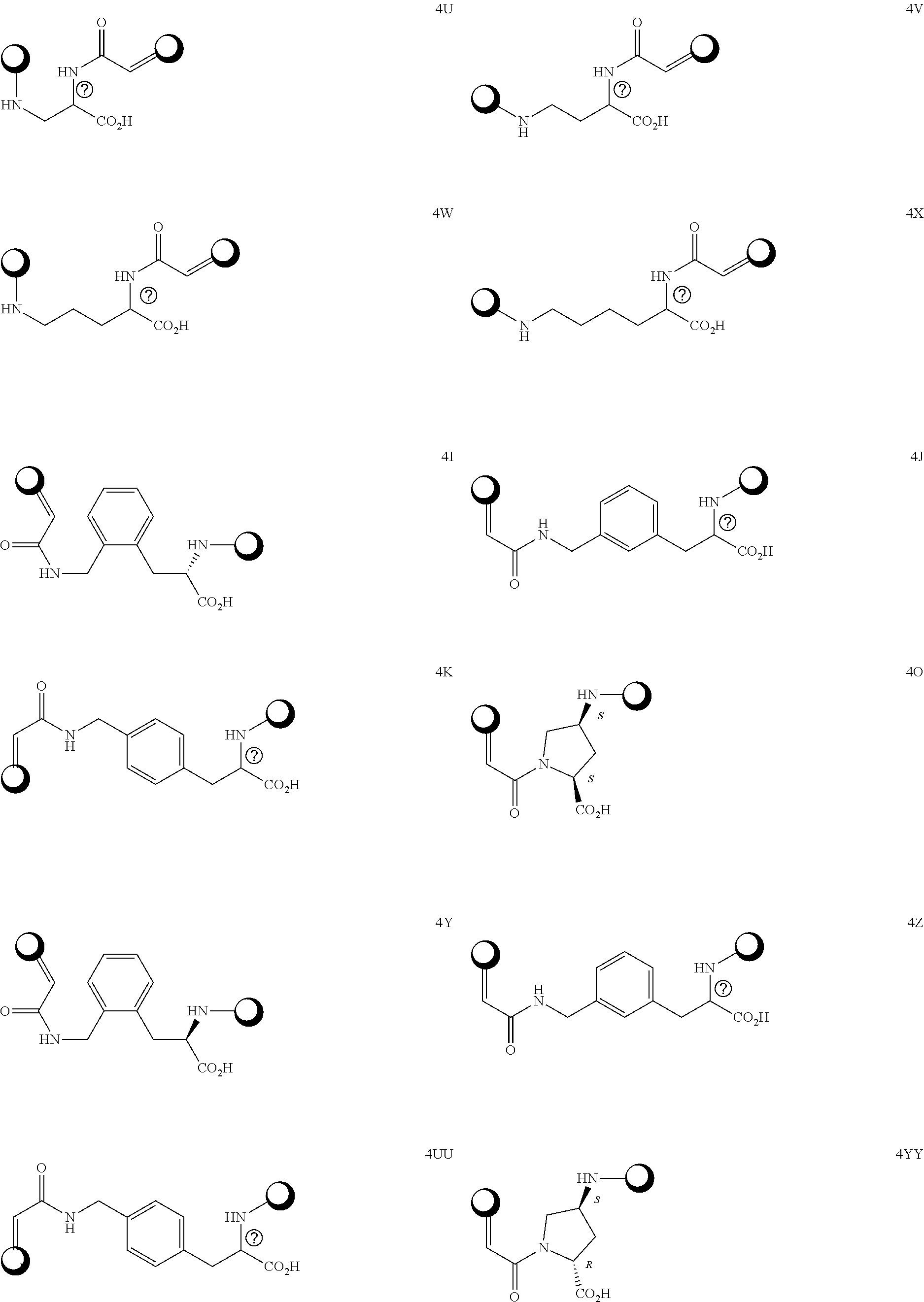

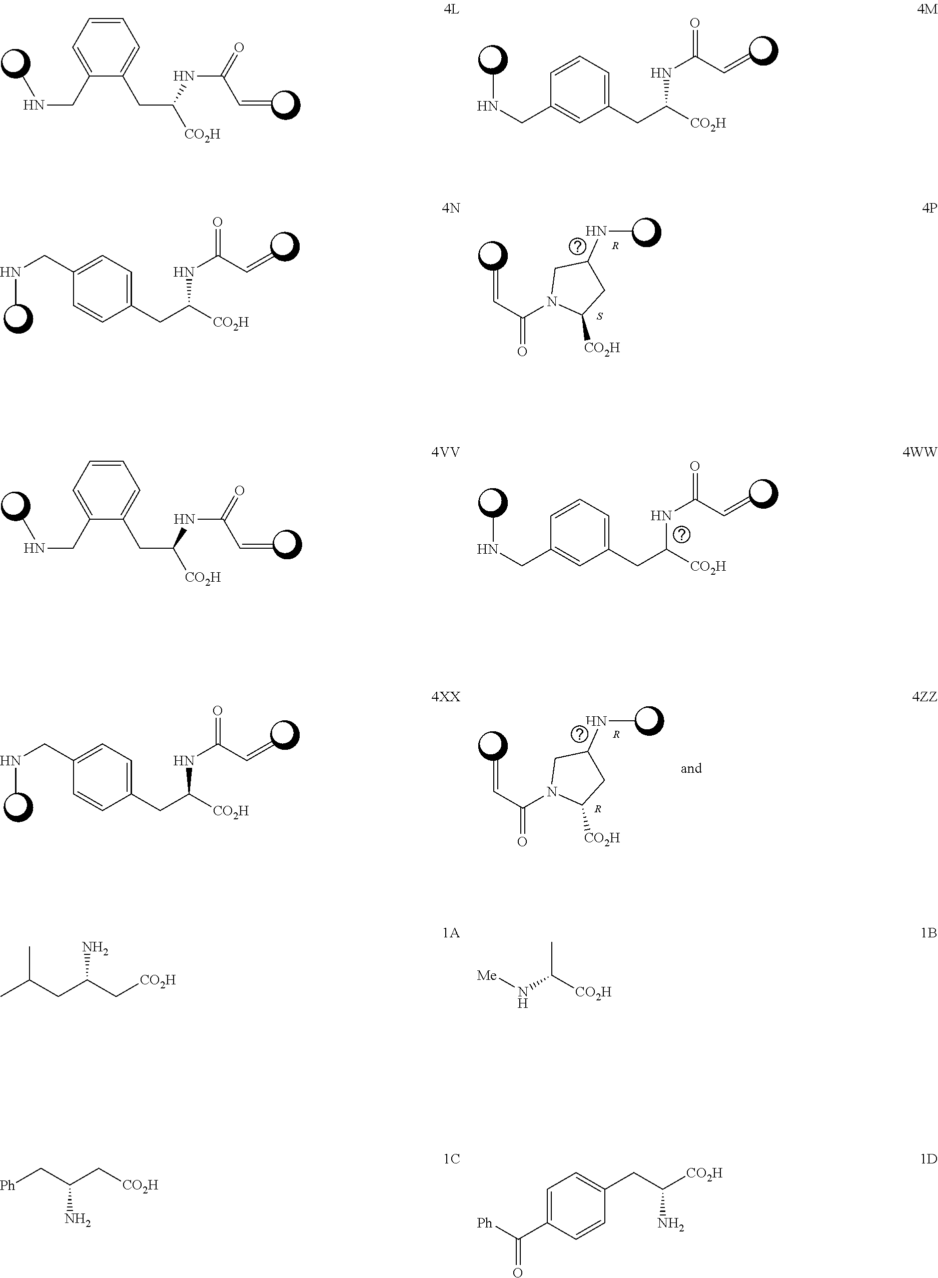

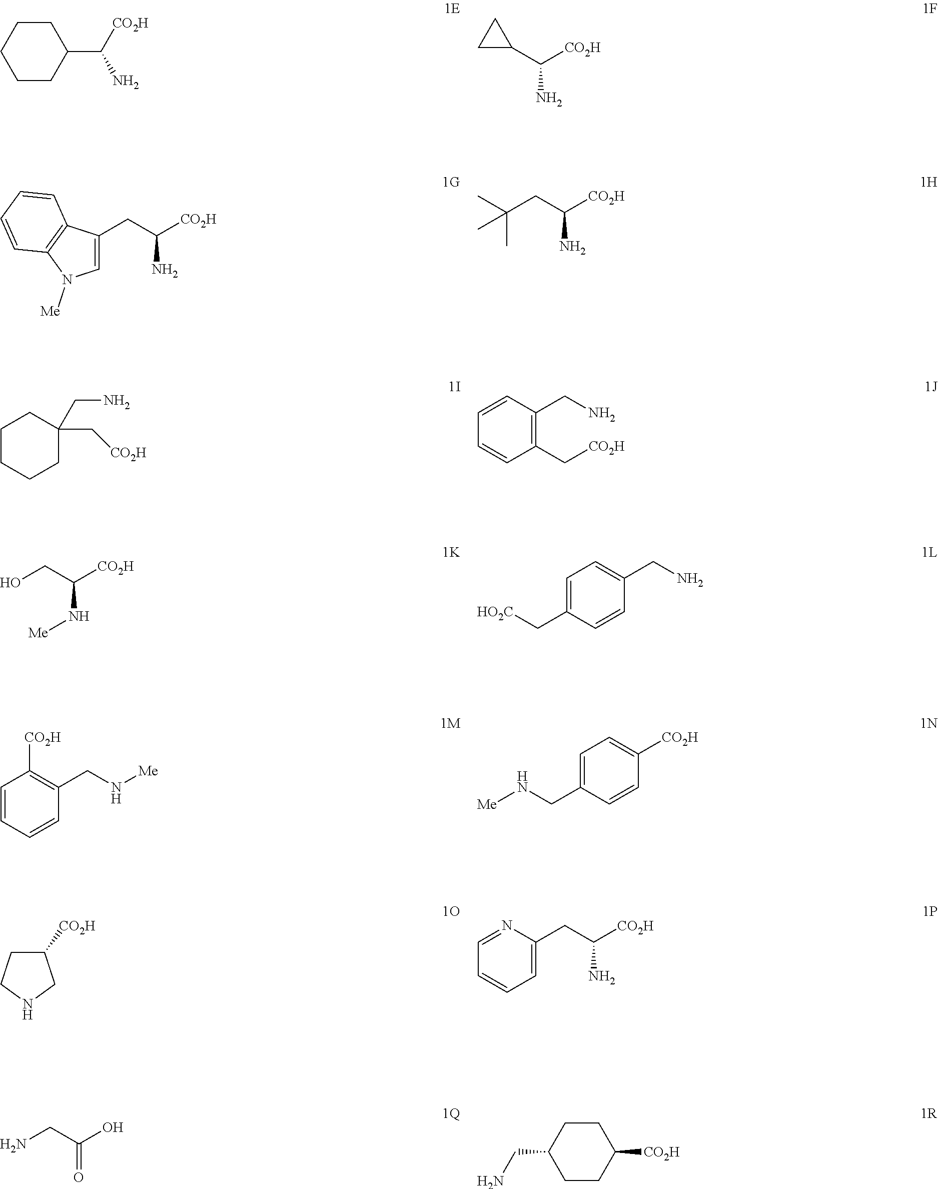

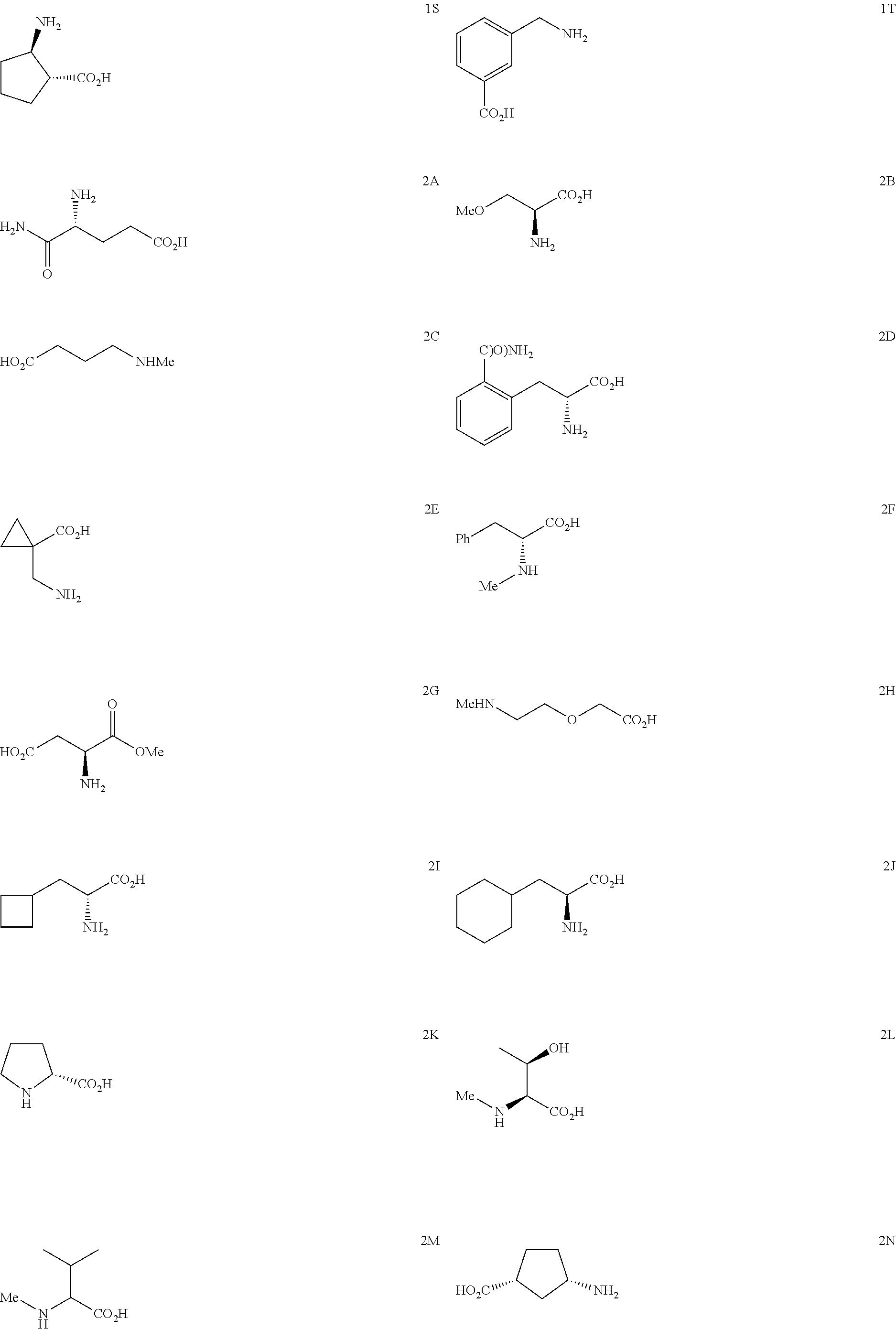

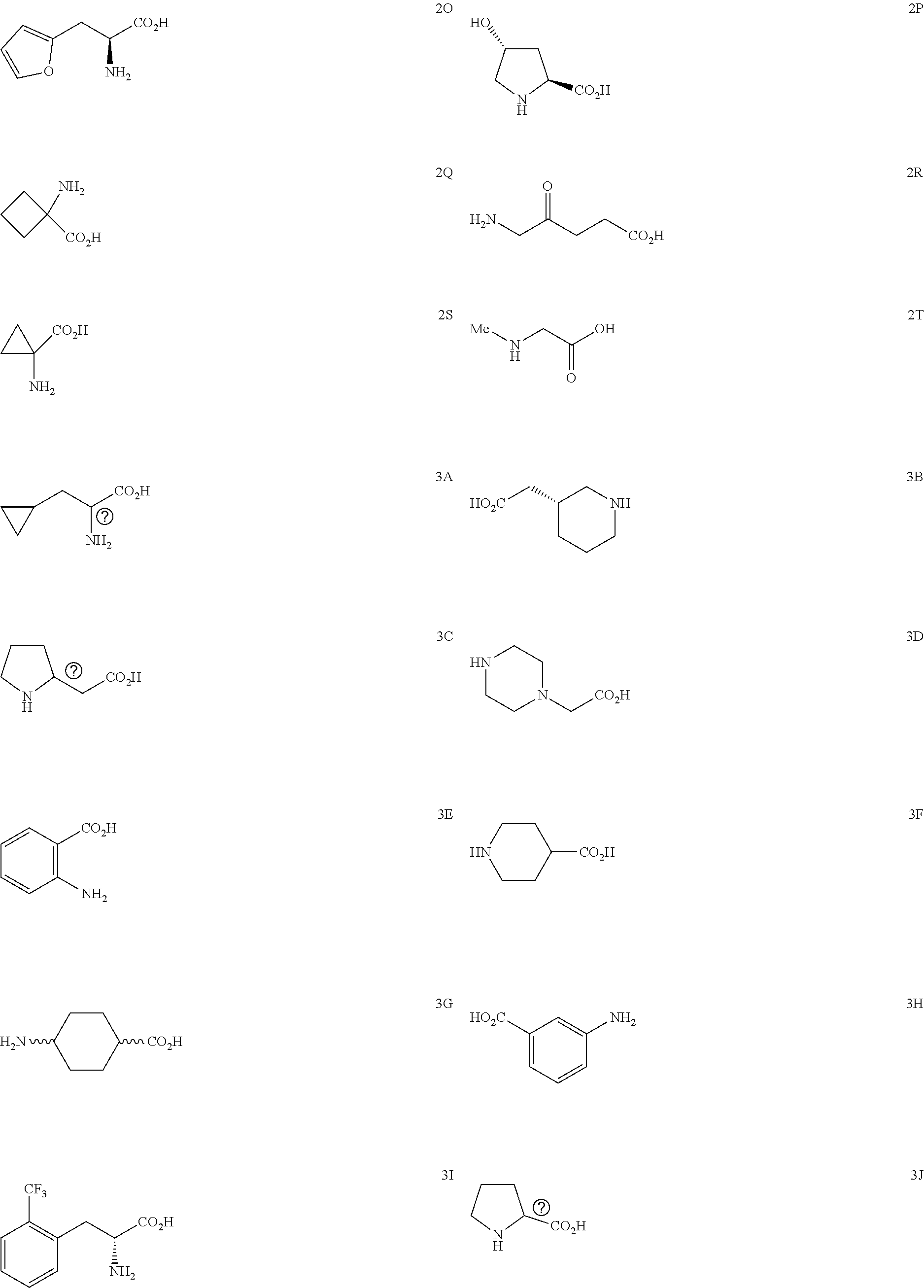

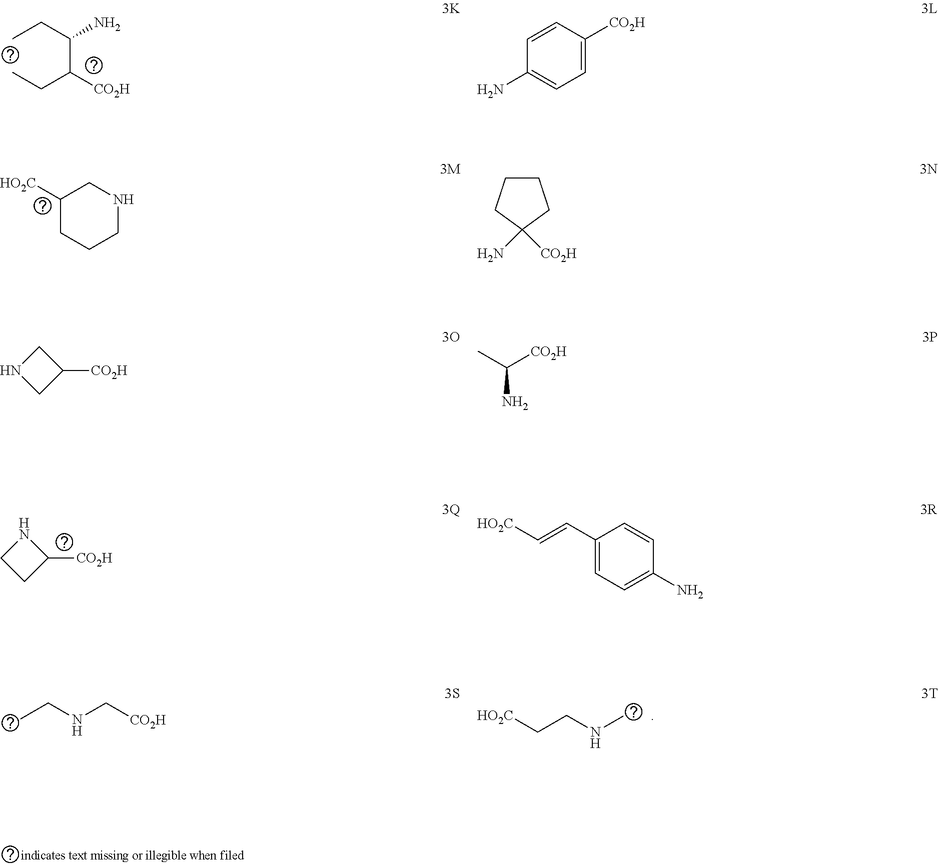

In certain embodiments, the compound of Formula (I′) or (I) is not a compound disclosed in Table 28 or FIG. 28 in PCT Application Publication no. WO 2019/168654, published Sep. 6, 2019. In certain embodiments, the compound of Formula (I′) or (I) is not a compound produced using DNA-templated synthesis and based on one or more codons from the orthogonal codon sets of Table 5A:

TABLE 5A

| 4A | 4B | 4C |
|---|---|---|
| 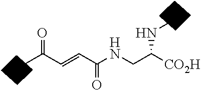 | 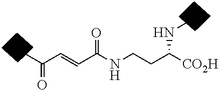 | 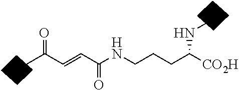 [SMILES: CN[C@@H](CCCNC(=O)/C=C/C(C)=O)C(=O)O] |
| L-Dap-s | L-Dab-s | L-Orn-s |
| 4E | 4F | 4G |
| 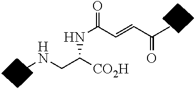 | 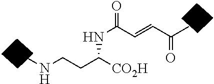 | 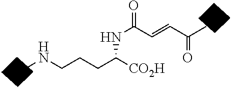 |
| L-Dap-a | L-Dab-a | L-Orn-a |
| 4I | 4J | 4K |
| 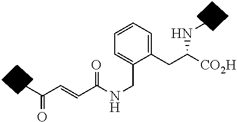 | 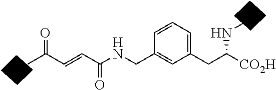 [SMILES: CN[C@@H](Cc1cccc(CNC(=O)/C=C/C(C)=O)c1)C(=O)O] | 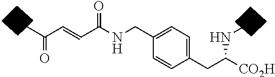 |
| L-2-NHCH₂-Phe-s | L-3-NHCH₂-Phe-s | L-4-NHCH₂-Phe-s |
| 4M | 4N | 4O |
| 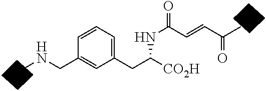 | 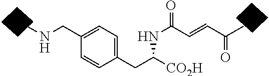 | 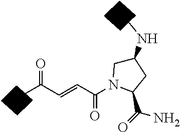 |
| L-3-NHCH,-Phe-a | L-4-NHCH₂-Phe-a | S,S-NH₂-Pro |
| 4Q | 4R | 4S |
| 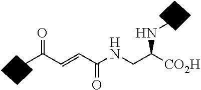 | 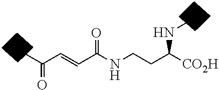 | 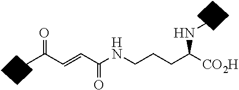 [SMILES: CN[C@H](CCCNC(=O)/C=C/C(C)=O)C(=O)O] |
| D-Dap-s | D-Dab-s | D-Orn-s |
| 4U | 4V | 4W |
| 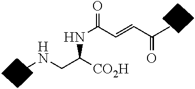 | 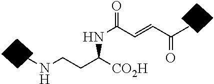 | 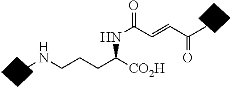 |
| D-Dap-a | D-Dab-a | D-Orn-a |
| 4Y | 4Z | 4UU |
| 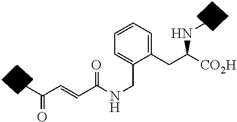 | 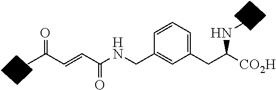 [SMILES: CN[C@H](Cc1cccc(CNC(=O)/C=C/C(C)=O)c1)C(=O)O] | 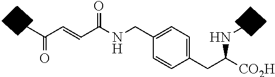 |
| D-2-NHCH₂-Phe-s | D-3-NHCH₂-Phe-s | D-4-NHCH₂-Phe-s |
| 4WW | 4XX | 4YY |
| 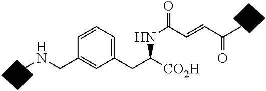 | 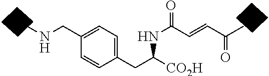 | 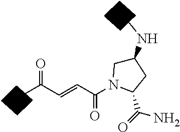 |
| D-3-NHCH₂-Phe-a | D-4-NHCH₂-Phe-a | R,S-NH₂-Pro |
| | 4A | 4D |
| | 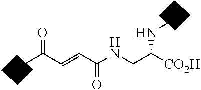 | 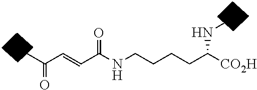 |
| | L-Dap-s | L-Lys-s |
| | 4E | 4H |
| | 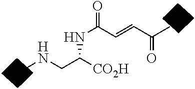 | 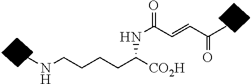 |
| | L-Dap-a | L-Lys-a |
| | 4I | 4L |
| | 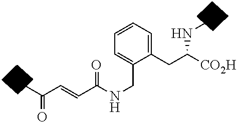 | 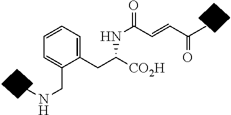 |
| | L-2-NHCH₂-Phe-s | L-2-NHCH₂-Phe-a |
| | 4M | 4P |
| | 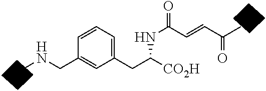 | 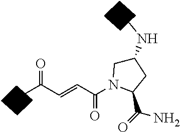 |
| | L-3-NHCH,-Phe-a | S,R-NH₂-Pro |
| | 4Q | 4T |
| | 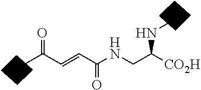 | 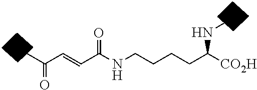 |
| | D-Dap-s | D-Lys-s |
| | 4U | 4X |
| | 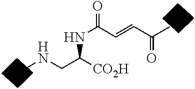 | 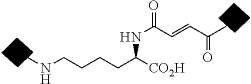 |
| | D-Dap-a | D-Lys-a |
| | 4Y | 4VV |
| | 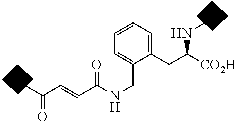 | 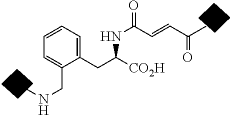 |
| | D-2-NHCH₂-Phe-s | D-2-NHCH₂-Phe-a |
| | 4WW | 4ZZ |
| | 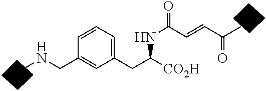 | 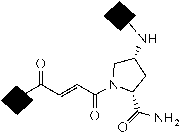 |
| | D-3-NHCH₂-Phe-a | R,R-NH₂-Pro |

and/or Table 7A:

TABLE 7A

| | | CAS | |
|---|---|---|---|
| | parent amino acid | number | commercial source |
| 1A | hexanoic acid, 3-amino-5-methyl-, (3S)- | 22818-43-5 | Astatech 52006 |
| 1B | N-methyl-D-alanine (HCl) | 29475-64-7 | Chem-Impex 04405 |
| 1C | D-beta-homophenylalanine (HCl) | 131270-08-1 | Peptech BD733-1 |
| 1D | D-4-benzoylphenylalanine | 201466-03-7 | Chem-Impex 05111 |
| 1E | 2-cyclohexyl-D-glycine | 14328-52-0 | Oakwood 040199-1g |
| 1F | D-cyclopropylglycine | 49607-01-4 | Chem-Impex 16797 |
| 1G | 1-methyl-L-tryptophan | 21339-55-9 | Sigma 447439-1G |
| 1H | L-neopentylglycine | 57224-50-7 | Chem-Impex 06234 |
| 1I | gabapentin | 60142-96-3 | Chem-Impex 16983 |
| 1J | 2-aminomethylphenylacetic Acid | 40851-65-8 | TCI America A2199 |
| 1K | N-methyl-L-serine (HCl) | 2480-26-4 | Chem-Impex 06357 |
| 1L | 4-(aminomethyl)benzeneacetic acid | 1200-05-1 | Astatech 75935 |
| 1M | 2-[(methylamino)methyl]benzoic acid | 527705-23-3 | Matrix Scientific 077672 |
| 1N | 4-[(methylamino)methyl]benzoic acid | 96084-38-7 | Matrix Scientific 065416 |
| 1O | L-beta-Proline | 72580-53-1 | Alfa Aesar H57895 |
| 1P | 3-(2-pyridyl)-D-alanine | 37535-52-7 | Peptech AD185-1 |
| 1Q | glycine | 56-40-6 | Sigma-Aldrich |
| 1R | tranexamic acid | 1197-18-8 | Chem-Impex 06911 |
| 1S | (1R,2R)-boc-2-aminocyclopentane | 245115-25-7 | Chem-Impex 14468 |
| | carboxylic acid | | |
| 1T | 3-aminomethylbenzoic acid | 2393-20-6 | Chem-Impex 28733. |

In certain embodiments, the compound of Formula (I′) or (I) is not a compound produced using DNA-templated synthesis and based on one or more codons from the orthogonal codon sets of Table 5A. In certain embodiments, the compound of Formula (I′) or (I) is not a compound produced using DNA-templated synthesis and based on one or more codons from the orthogonal codon sets of Table 7A. In certain embodiments, the compound of Formula (I′) or (I) is not a compound produced using DNA-templated synthesis and based on one or more codons from the orthogonal codon sets of Tables 5A, 7A, and/or anticodons or reagents in Table 6A:

TABLE 6A

| | |
|---|---|
| 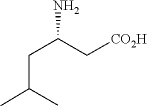 | 1A |
| 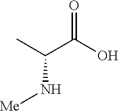 | 1B |
| 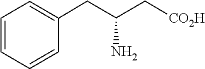 [SMILES: N[C@@H](CC(=O)O)Cc1ccccc1] | 1C |
| 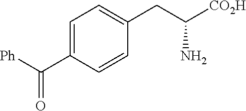 [SMILES: N[C@H](Cc1ccc(C(=O)c2ccccc2)cc1)C(=O)O] | 1D |
| 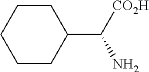 [SMILES: N[C@@H](C(=O)O)C1CCCCC1] | 1E |
| 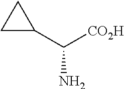 | 1F |
| 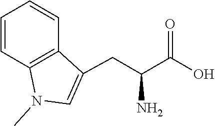 | 1G |
| 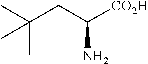 | 1H |
| 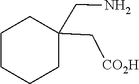 [SMILES: NCC1(CC(=O)O)CCCCC1] | 1I |
| 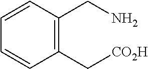 [SMILES: NCc1ccccc1CC(=O)O] | 1J |
| 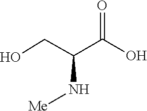 | 1K |
| 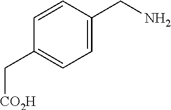 [SMILES: NCc1ccc(CC(=O)O)cc1] | 1L |
| 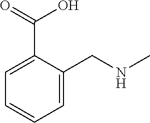 | 1M |
| 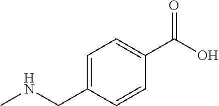 | 1N |
| 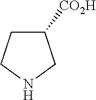 | 1O |
| 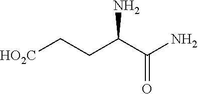 | 2A |
| 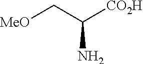 | 2B |
| 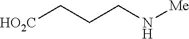 | 2C |
| 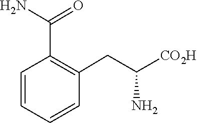 [SMILES: NC(=O)c1ccccc1C[C@@H](N)C(=O)O] | 2D |
| 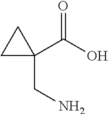 | 2E |
| 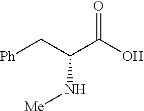 [SMILES: CN[C@H](Cc1ccccc1)C(=O)O] | 2F |
| 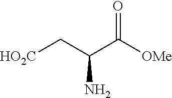 | 2G |
| 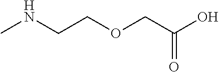 | 2H |
| 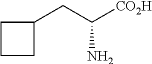 | 2I |
| 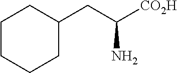 [SMILES: N[C@@H](CC1CCCCC1)C(=O)O] | 2J |
| 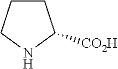 | 2K |
| 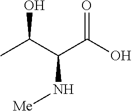 | 2L |
| 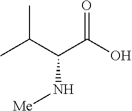 | 2M |
| 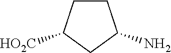 | 2N |
| 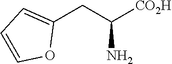 [SMILES: N[C@@H](Cc1ccco1)C(=O)O] | 2O |
| 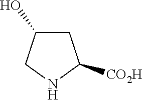 | 2P |
| 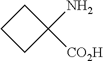 | 2Q |
| 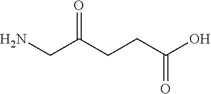 | 2R |
| 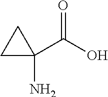 | 2S |
| 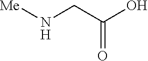 | 2T |
| 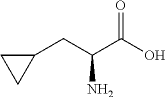 | 3A |
| 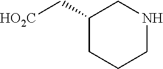 | 3B |
| 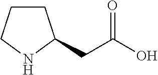 | 3C |
| 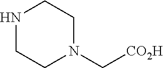 | 3D |
| 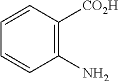 | 3E |
| 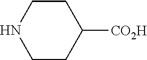 | 3F |
| 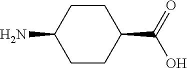 | 3G |
| 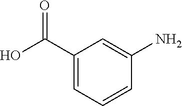 | 3H |
| 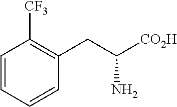 [SMILES: N[C@H](Cc1ccccc1C(F)(F)F)C(=O)O] | 3I |
| 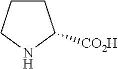 | 3J |
| 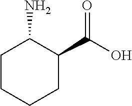 | 3K |
| 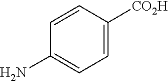 | 3L |
| 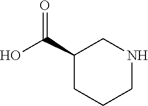 | 3M |
| 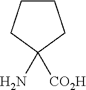 | 3N |
| 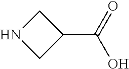 | 3O |
| 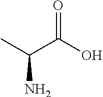 | 3P |
| 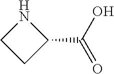 | 3Q |
| 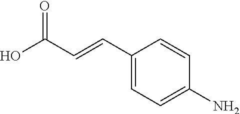 [SMILES: Nc1ccc(/C=C/C(=O)O)cc1] | 3R |
| 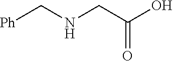 [SMILES: O=C(O)CNCc1ccccc1] | 3S |
| 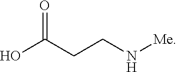 | 3T |

In certain embodiments, the compound of Formula (I′) or (I) is not a compound produced using DNA-templated synthesis and based on one or more codons from Table 6A.

In one aspect, the present disclosure provides compounds of Formula (I):

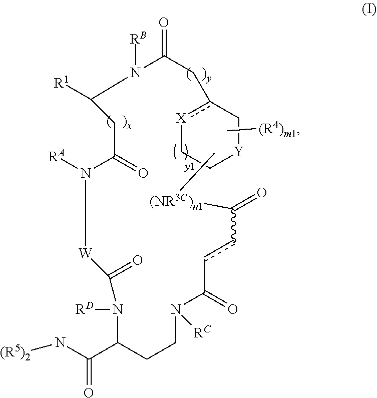

and pharmaceutically acceptable salts, solvates, hydrates, polymorphs, co-crystals, tautomers, stereoisomers, isotopically labeled derivatives, and prodrugs thereof, wherein R¹, R^{3C}, R⁴, R⁵, R^{A}, R^{B}, R^{C}, R^{D}, x, y, y1, m1, n1, W, X, and Y are as defined herein. In certain embodiments, the compound of Formula (I′) or (I) is not a compound disclosed in PCT Application Publication no. WO 2019/168654, published Sep. 6, 2019.

Exemplary compounds of Formula (I′) or (I) include, but are not limited to:

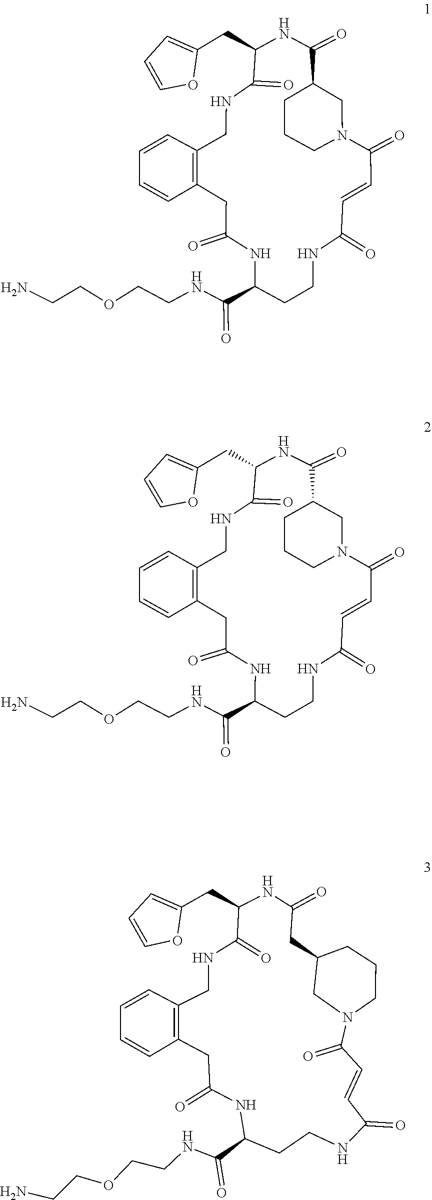 [SMILES: NCCOCCNC(=O)[C@@H]1CCNC(=O)/C=C/C(=O)N2CCC[C@H](C2)C(=O)N[C@H](Cc2ccco2)C(=O)NCc2ccccc2CC(=O)N1 ; NCCOCCNC(=O)[C@@H]1CCNC(=O)/C=C/C(=O)N2CCC[C@@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@H](CC(=O)N[C@H](Cc3ccco3)C(=O)NCc3ccccc3CC(=O)N1)C2]

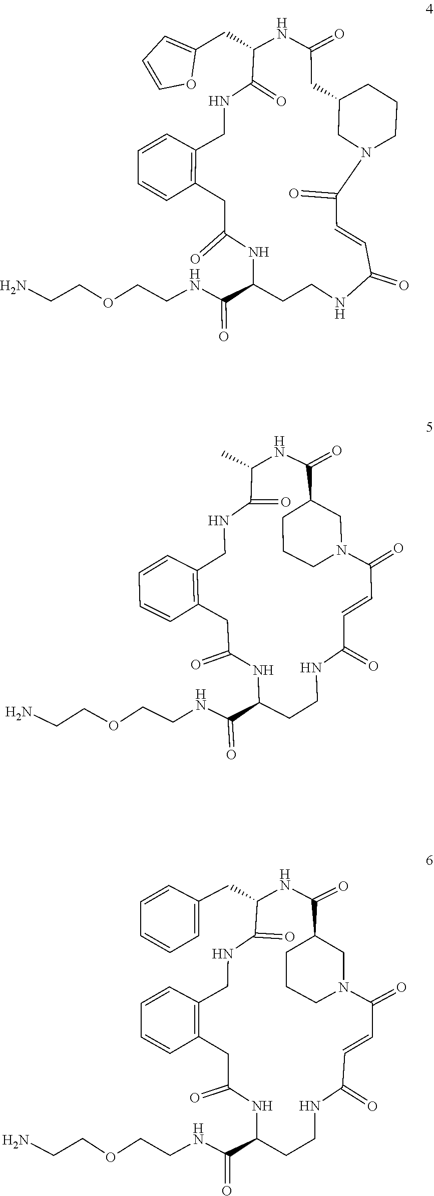 [SMILES: NCCOCCNC(=O)[C@@H]1CCNC(=O)/C=C/C(=O)N2CCC[C@@H](CC(=O)N[C@@H](Cc3ccco3)C(=O)NCc3ccccc3CC(=O)N1)C2 ; C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccccc2)C(=O)NCc2ccccc2CC(=O)N1]

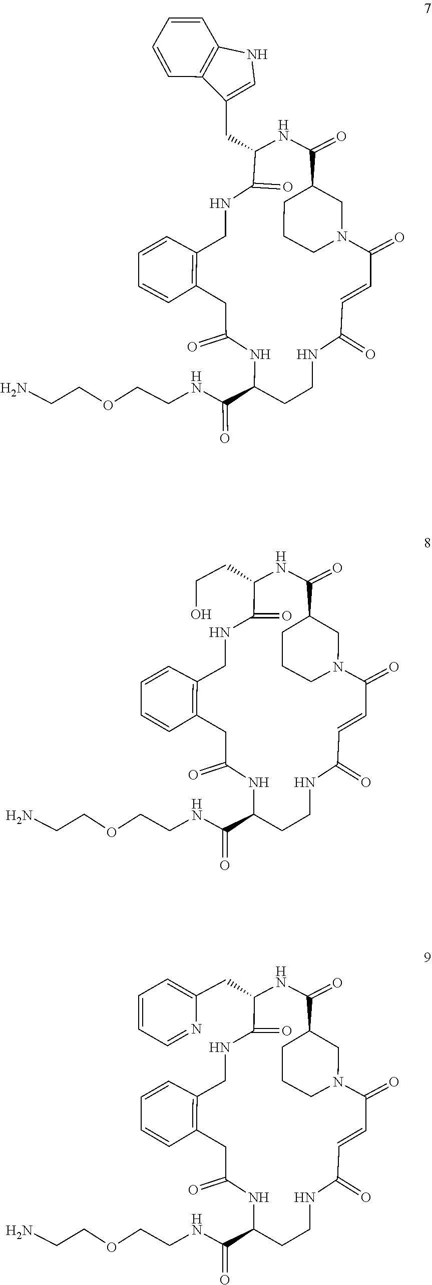 [SMILES: NCCOCCNC(=O)[C@@H]1CCNC(=O)/C=C/C(=O)N2CCC[C@H](C2)C(=O)N[C@@H](Cc2c[nH]c3ccccc23)C(=O)NCc2ccccc2CC(=O)N1 ; NCCOCCNC(=O)[C@@H]1CCNC(=O)/C=C/C(=O)N2CCC[C@H](C2)C(=O)N[C@@H](CCO)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccccn2)C(=O)NCc2ccccc2CC(=O)N1]

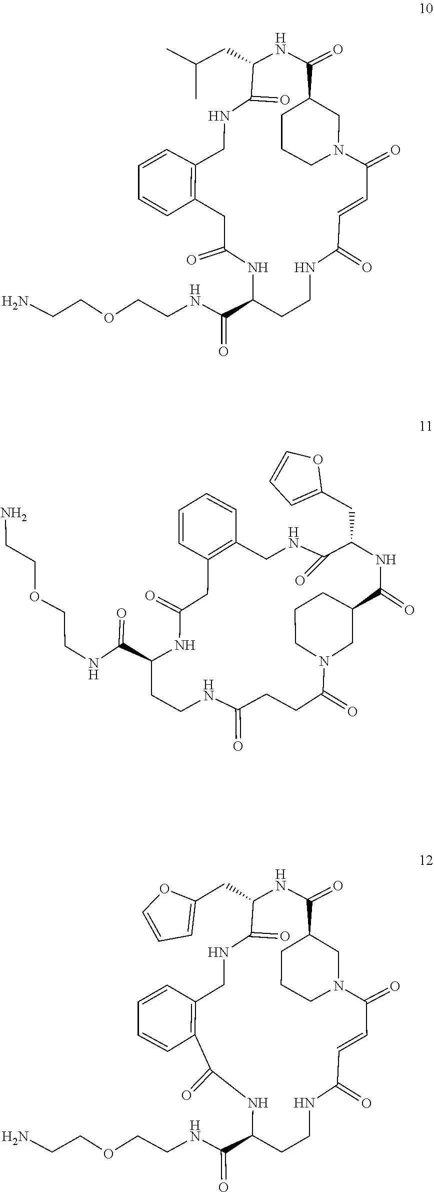 [SMILES: CC(C)C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)CCC(=O)N2CCC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccco2)C(=O)NCc2ccccc2C(=O)N1]

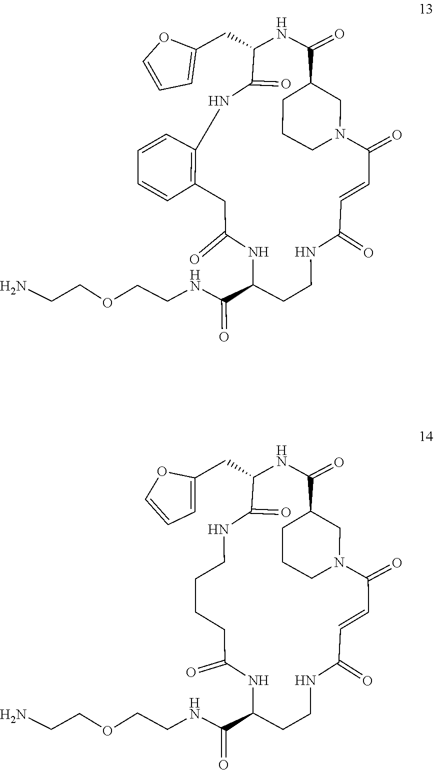 [SMILES: NCCOCCNC(=O)[C@@H]1CCNC(=O)/C=C/C(=O)N2CCC[C@H](C2)C(=O)N[C@@H](Cc2ccco2)C(=O)Nc2ccccc2CC(=O)N1 ; NCCOCCNC(=O)[C@@H]1CCNC(=O)/C=C/C(=O)N2CCC[C@H](C2)C(=O)N[C@@H](Cc2ccco2)C(=O)NCCCCC(=O)N1]

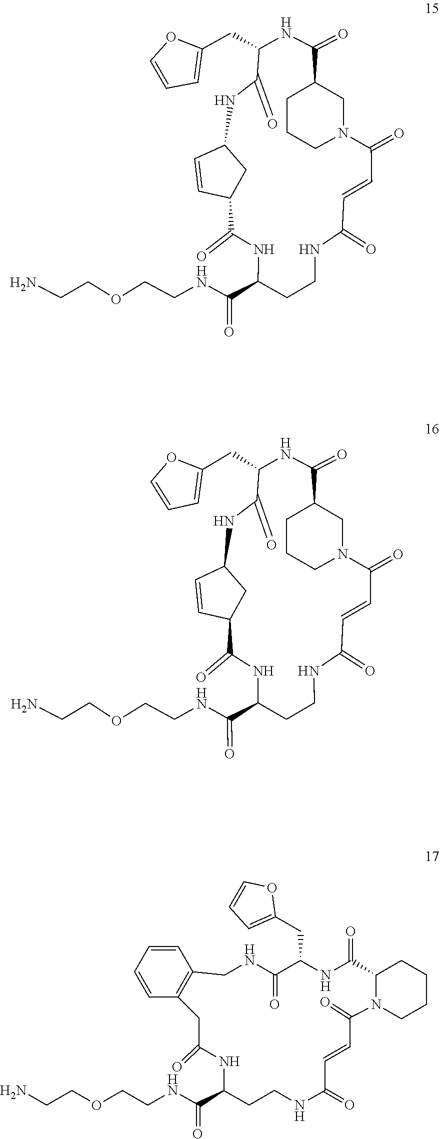 [SMILES: NCCOCCNC(=O)[C@@H]1CCNC(=O)/C=C/C(=O)N2CCC[C@H](C2)C(=O)N[C@@H](Cc2ccco2)C(=O)N[C@H]2C=C[C@H](C2)C(=O)N1 ; NCCOCCNC(=O)[C@@H]1CCNC(=O)/C=C/C(=O)N2CCC[C@H](C2)C(=O)N[C@@H](Cc2ccco2)C(=O)N[C@@H]2C=C[C@@H](C2)C(=O)N1 ; NCCOCCNC(=O)[C@@H]1CCNC(=O)/C=C/C(=O)N2CCCC[C@H]2C(=O)N[C@@H](Cc2ccco2)C(=O)NCc2ccccc2CC(=O)N1]

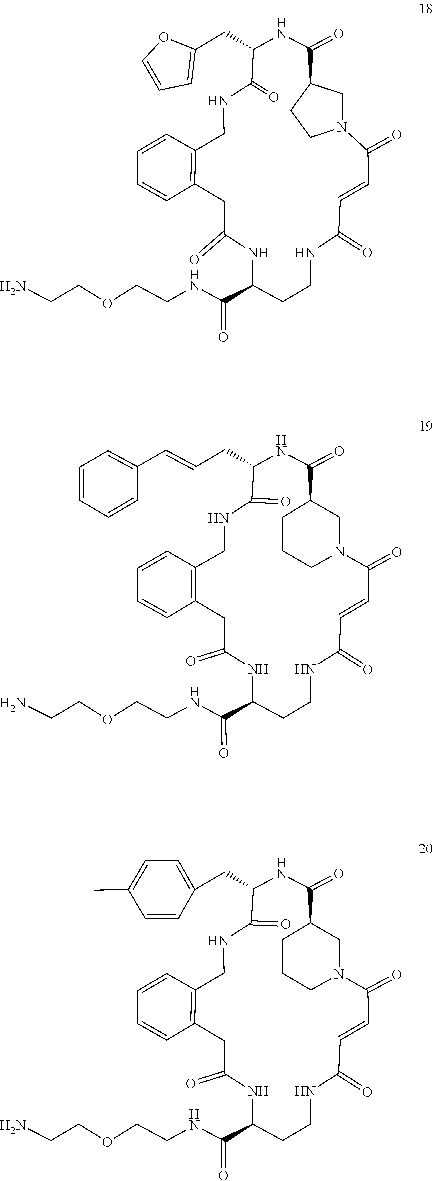 [SMILES: NCCOCCNC(=O)[C@@H]1CCNC(=O)/C=C/C(=O)N2CC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@H](C2)C(=O)N[C@@H](C/C=C/c2ccccc2)C(=O)NCc2ccccc2CC(=O)N1 ; Cc1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1]

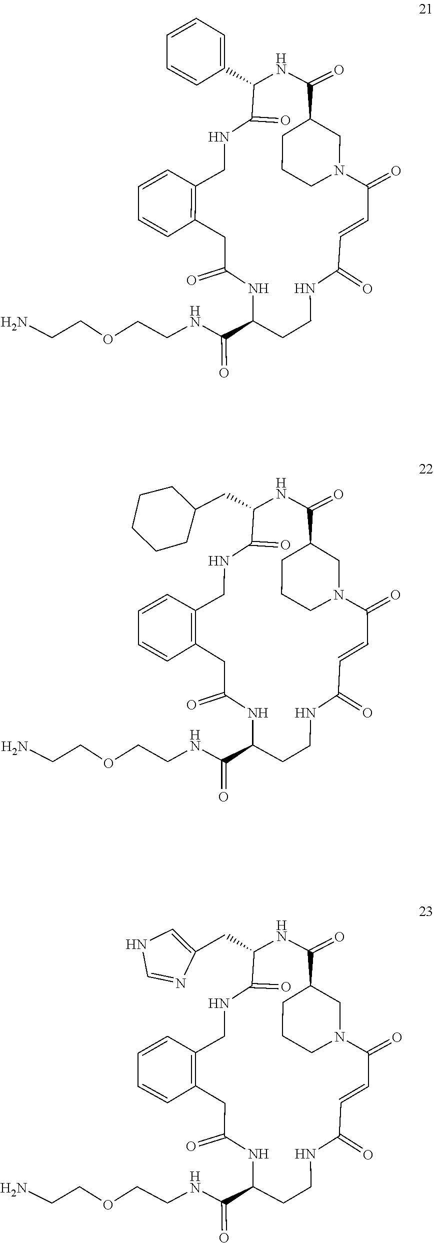 [SMILES: NCCOCCNC(=O)[C@@H]1CCNC(=O)/C=C/C(=O)N2CCC[C@H](C2)C(=O)N[C@@H](c2ccccc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](CC2CCCCC2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2c[nH]cn2)C(=O)NCc2ccccc2CC(=O)N1]

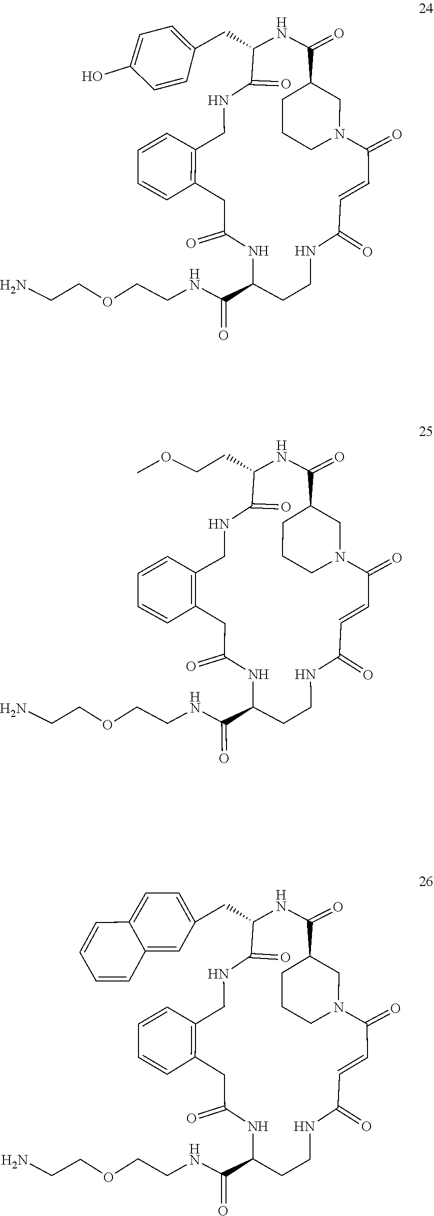 [SMILES: NCCOCCNC(=O)[C@@H]1CCNC(=O)/C=C/C(=O)N2CCC[C@H](C2)C(=O)N[C@@H](Cc2ccc(O)cc2)C(=O)NCc2ccccc2CC(=O)N1 ; COCC[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccc3ccccc3c2)C(=O)NCc2ccccc2CC(=O)N1]

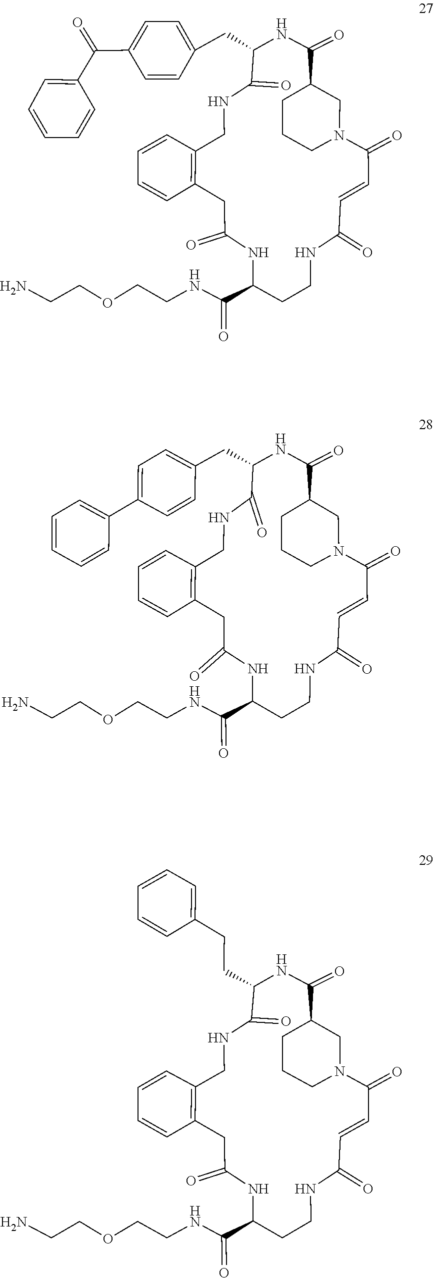 [SMILES: NCCOCCNC(=O)[C@@H]1CCNC(=O)/C=C/C(=O)N2CCC[C@H](C2)C(=O)N[C@@H](Cc2ccc(C(=O)c3ccccc3)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(-c3ccccc3)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](CCc2ccccc2)C(=O)NCc2ccccc2CC(=O)N1]

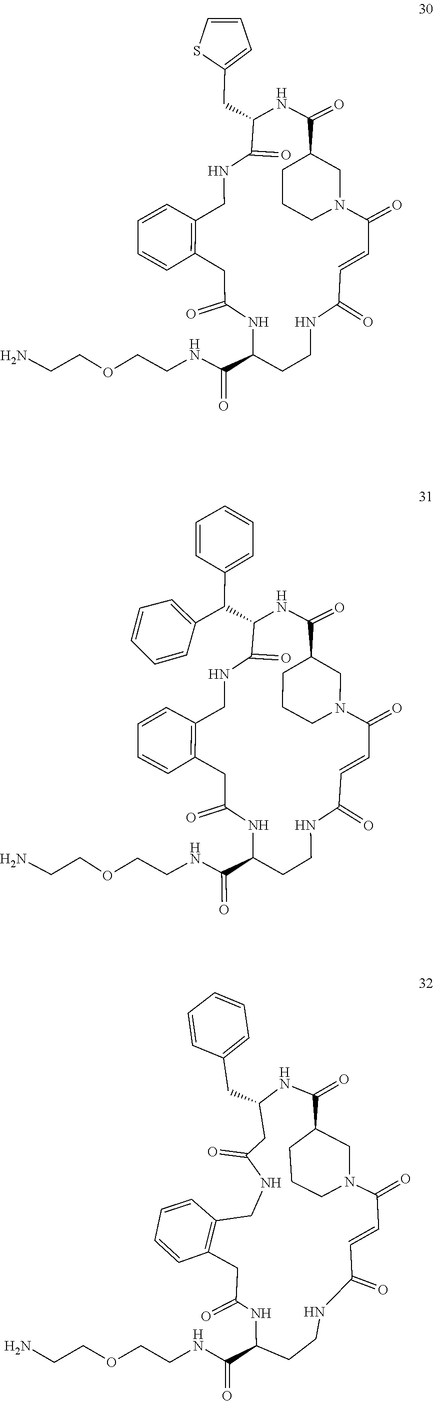 [SMILES: NCCOCCNC(=O)[C@@H]1CCNC(=O)/C=C/C(=O)N2CCC[C@H](C2)C(=O)N[C@@H](Cc2cccs2)C(=O)NCc2ccccc2CC(=O)N1 ; NCCOCCNC(=O)[C@@H]1CCNC(=O)/C=C/C(=O)N2CCC[C@H](C2)C(=O)N[C@@H](C(c2ccccc2)c2ccccc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccccc2)CC(=O)NCc2ccccc2CC(=O)N1]

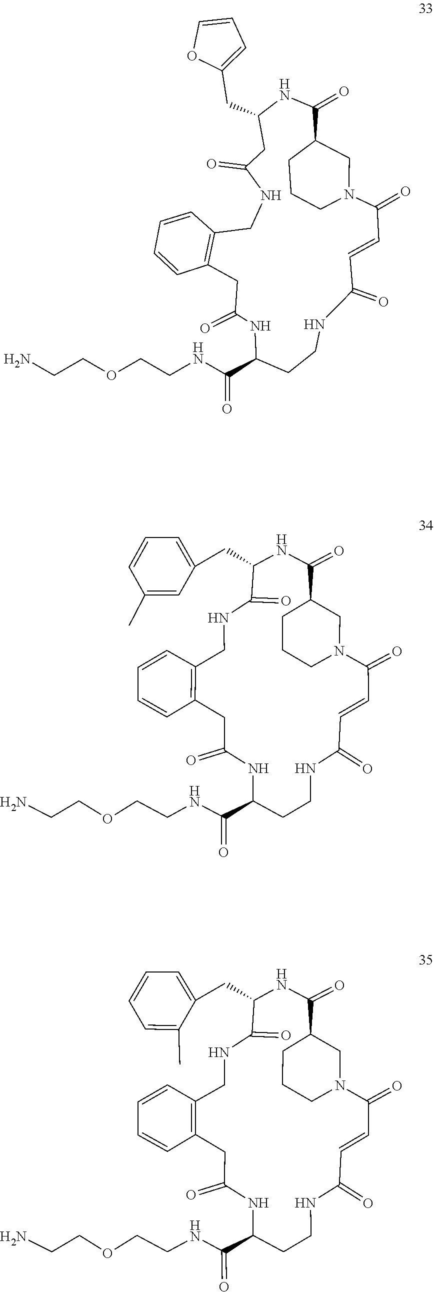 [SMILES: NCCOCCNC(=O)[C@@H]1CCNC(=O)/C=C/C(=O)N2CCC[C@H](C2)C(=O)N[C@@H](Cc2ccco2)CC(=O)NCc2ccccc2CC(=O)N1 ; Cc1cccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)c1 ; Cc1ccccc1C[C@@H]1NC(=O)[C@@H]2CCCN(C2)C(=O)/C=C/C(=O)NCC[C@@H](C(=O)NCCOCCN)NC(=O)Cc2ccccc2CNC1=O]

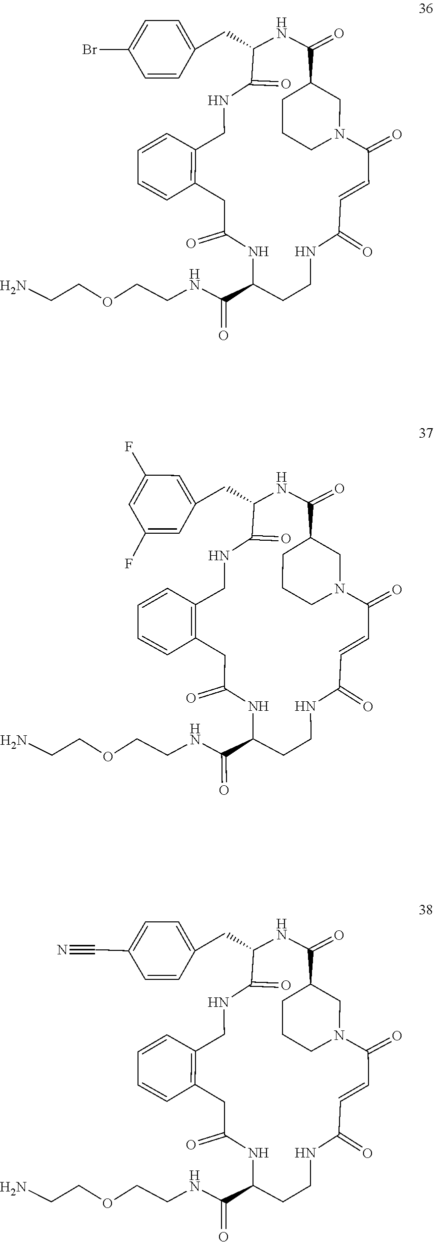 [SMILES: NCCOCCNC(=O)[C@@H]1CCNC(=O)/C=C/C(=O)N2CCC[C@H](C2)C(=O)N[C@@H](Cc2ccc(Br)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2cc(F)cc(F)c2)C(=O)NCc2ccccc2CC(=O)N1 ; N#Cc1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1]

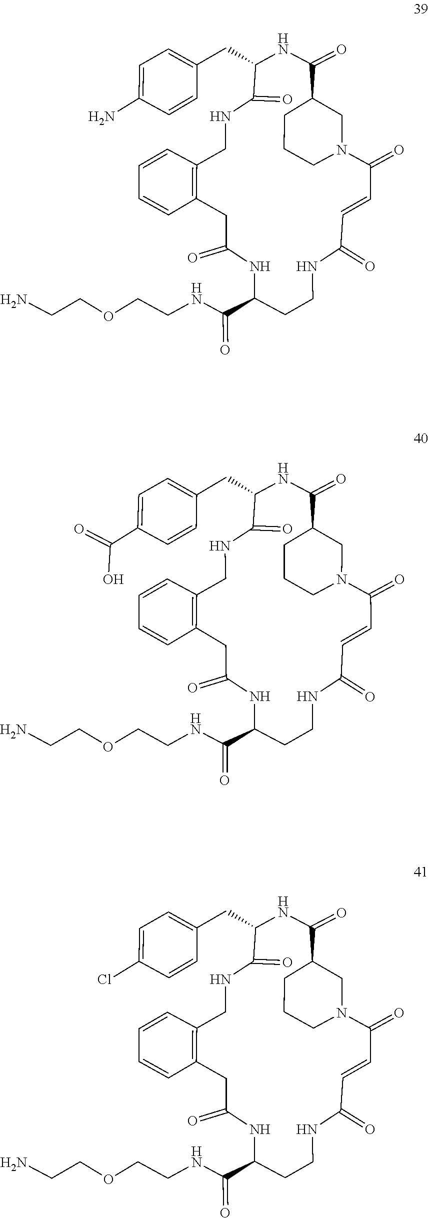 [SMILES: NCCOCCNC(=O)[C@@H]1CCNC(=O)/C=C/C(=O)N2CCC[C@H](C2)C(=O)N[C@@H](Cc2ccc(N)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(C(=O)O)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(Cl)cc2)C(=O)NCc2ccccc2CC(=O)N1]

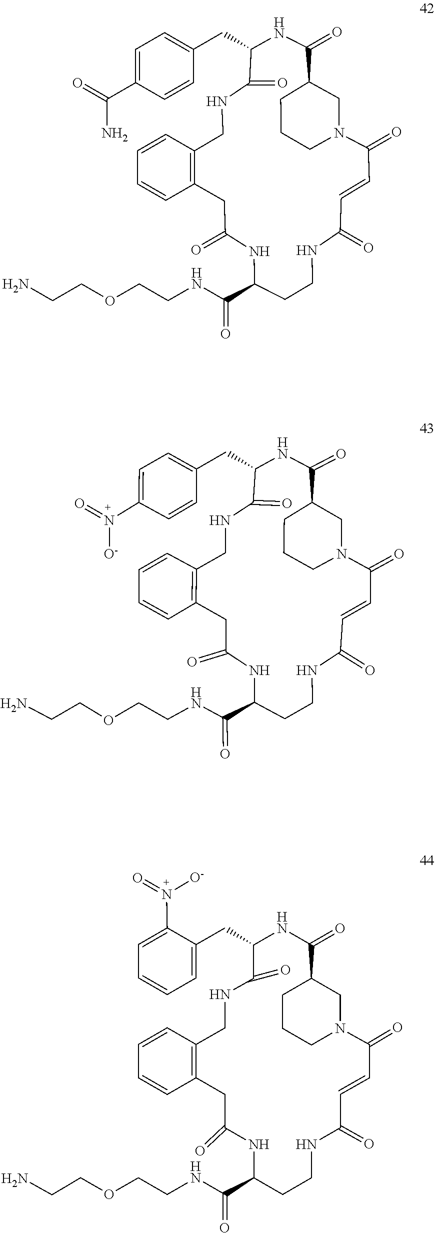

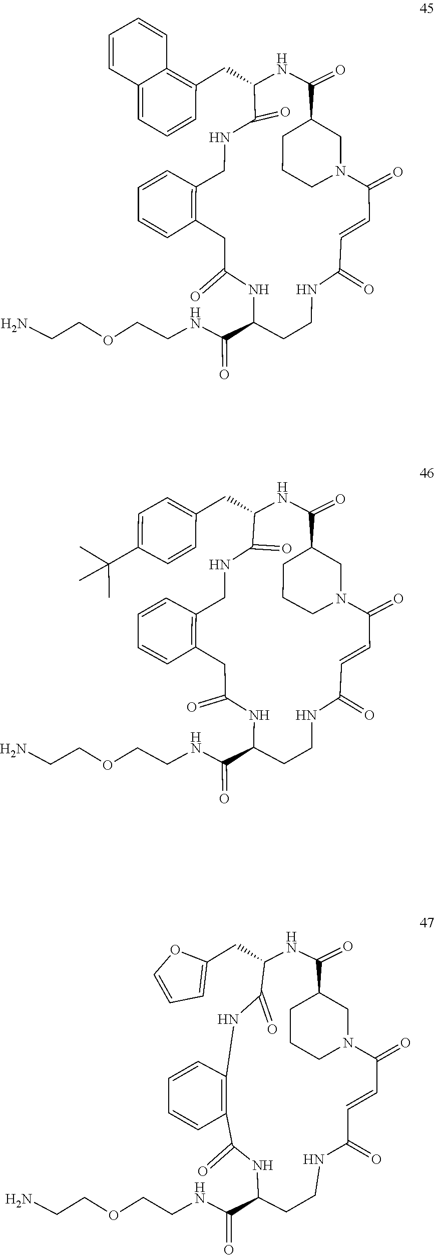 [SMILES: NCCOCCNC(=O)[C@@H]1CCNC(=O)/C=C/C(=O)N2CCC[C@H](C2)C(=O)N[C@@H](Cc2cccc3ccccc23)C(=O)NCc2ccccc2CC(=O)N1 ; CC(C)(C)c1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@H](C2)C(=O)N[C@@H](Cc2ccco2)C(=O)Nc2ccccc2C(=O)N1]

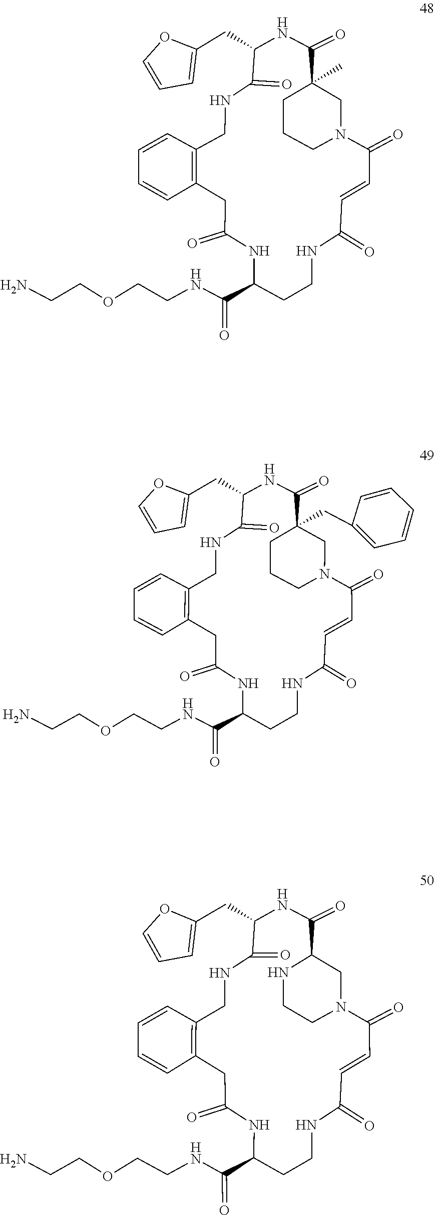 [SMILES: C[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; NCCOCCNC(=O)[C@@H]1CCNC(=O)/C=C/C(=O)N2CCC[C@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCN[C@H](C2)C(=O)N[C@@H](Cc2ccco2)C(=O)NCc2ccccc2CC(=O)N1]

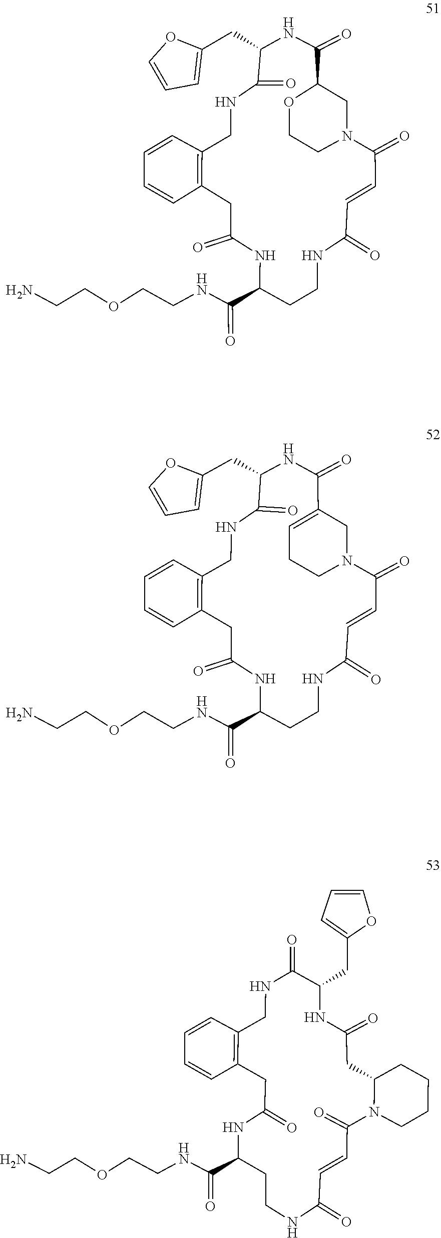 [SMILES: NCCOCCNC(=O)[C@@H]1CCNC(=O)/C=C/C(=O)N2CCO[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC=C(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCCC[C@H]2CC(=O)N[C@@H](Cc2ccco2)C(=O)NCc2ccccc2CC(=O)N1]

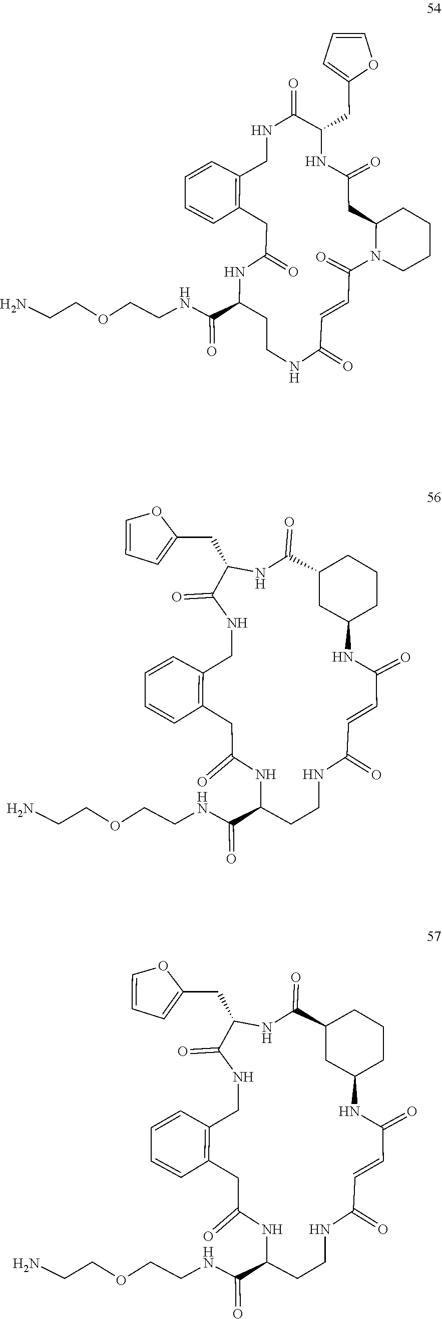 [SMILES: NCCOCCNC(=O)[C@@H]1CCNC(=O)/C=C/C(=O)N2CCCC[C@@H]2CC(=O)N[C@@H](Cc2ccco2)C(=O)NCc2ccccc2CC(=O)N1 ; NCCOCCNC(=O)[C@@H]1CCNC(=O)/C=C/C(=O)N[C@@H]2CCC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N[C@@H]2CCC[C@@H](C2)C(=O)N[C@@H](Cc2ccco2)C(=O)NCc2ccccc2CC(=O)N1]

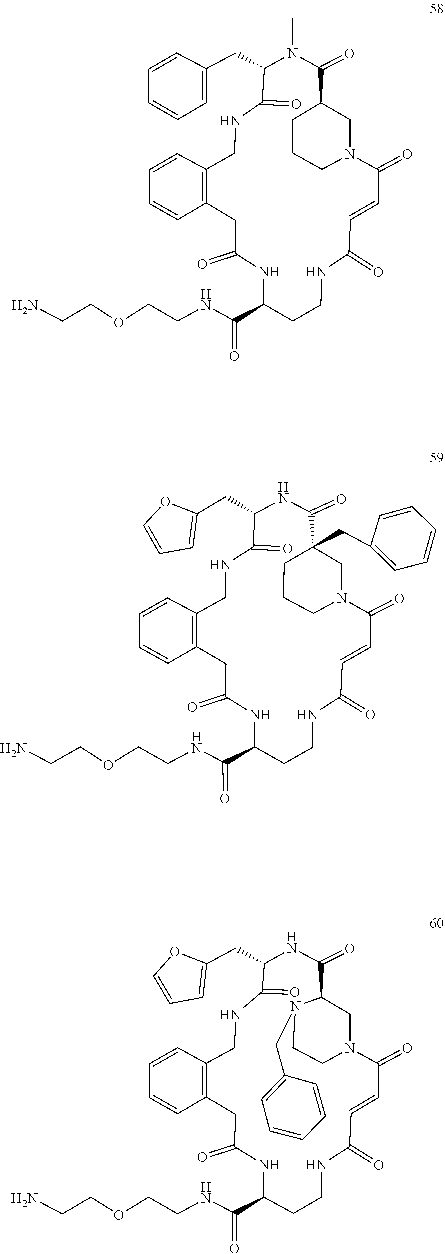 [SMILES: CN1C(=O)[C@@H]2CCCN(C2)C(=O)/C=C/C(=O)NCC[C@@H](C(=O)NCCOCCN)NC(=O)Cc2ccccc2CNC(=O)[C@@H]1Cc1ccccc1 ; NCCOCCNC(=O)[C@@H]1CCNC(=O)/C=C/C(=O)N2CCC[C@@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCN(Cc3ccccc3)[C@H](C2)C(=O)N[C@@H](Cc2ccco2)C(=O)NCc2ccccc2CC(=O)N1]

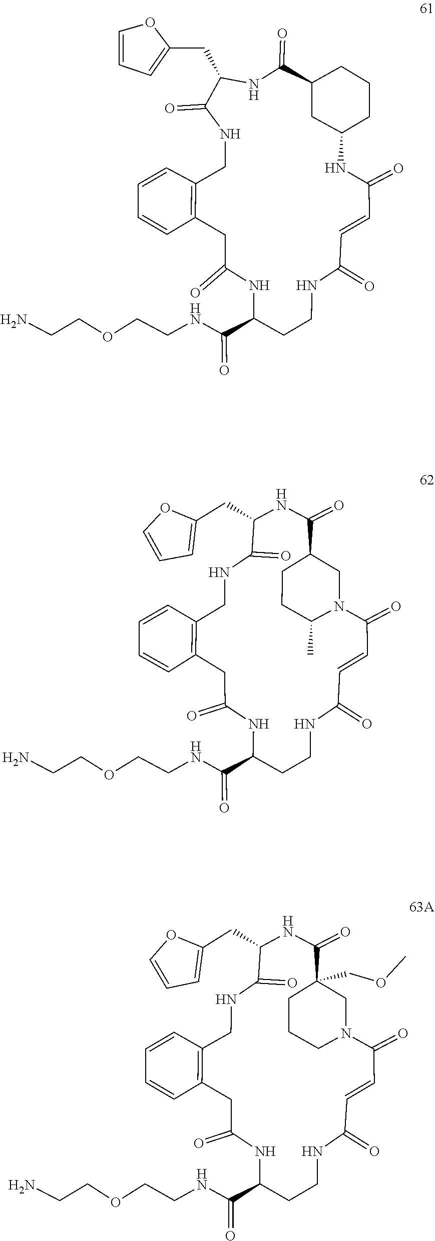 [SMILES: NCCOCCNC(=O)[C@@H]1CCNC(=O)/C=C/C(=O)N[C@H]2CCC[C@@H](C2)C(=O)N[C@@H](Cc2ccco2)C(=O)NCc2ccccc2CC(=O)N1 ; C[C@@H]1CC[C@@H]2CN1C(=O)/C=C/C(=O)NCC[C@@H](C(=O)NCCOCCN)NC(=O)Cc1ccccc1CNC(=O)[C@H](Cc1ccco1)NC2=O ; COC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

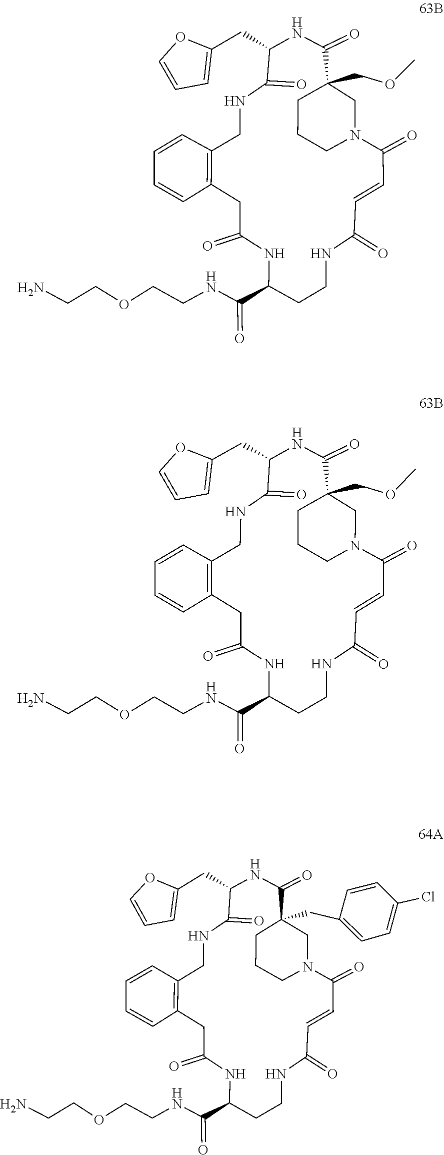 [SMILES: COC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; COC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; NCCOCCNC(=O)[C@@H]1CCNC(=O)/C=C/C(=O)N2CCC[C@](Cc3ccc(Cl)cc3)(C2)C(=O)N[C@@H](Cc2ccco2)C(=O)NCc2ccccc2CC(=O)N1]

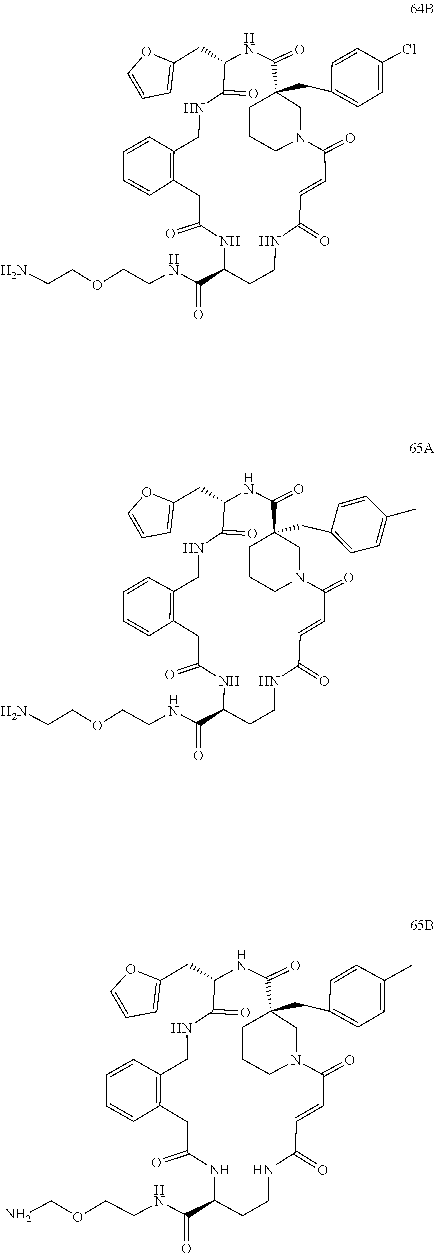 [SMILES: NCCOCCNC(=O)[C@@H]1CCNC(=O)/C=C/C(=O)N2CCC[C@@](Cc3ccc(Cl)cc3)(C2)C(=O)N[C@@H](Cc2ccco2)C(=O)NCc2ccccc2CC(=O)N1 ; Cc1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; Cc1ccc(C[C@]23CCCN(C2)C(=O)/C=C/C(=O)NCC[C@@H](C(=O)NCCOCN)NC(=O)Cc2ccccc2CNC(=O)[C@H](Cc2ccco2)NC3=O)cc1]

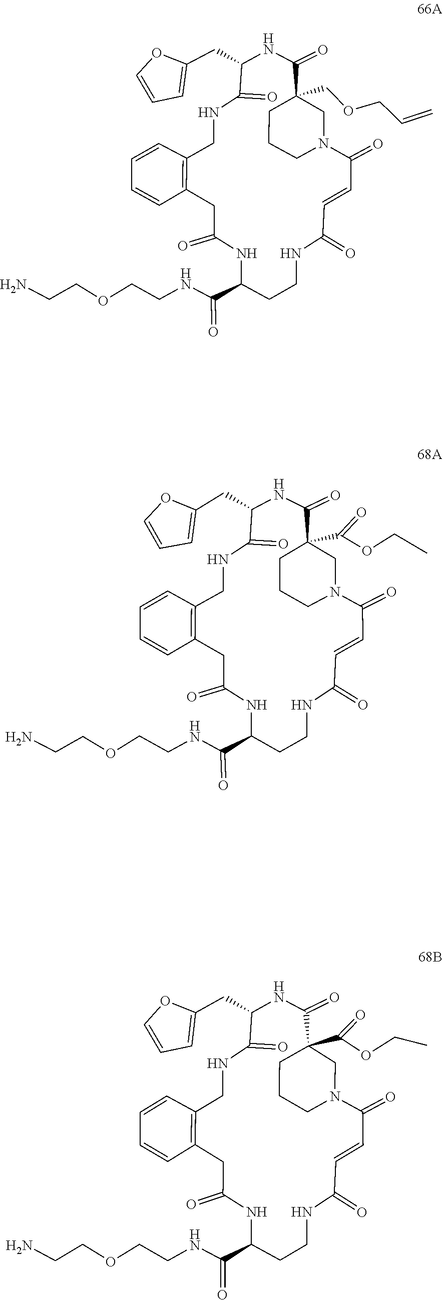 [SMILES: C=CCOC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CCOC(=O)[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CCOC(=O)[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

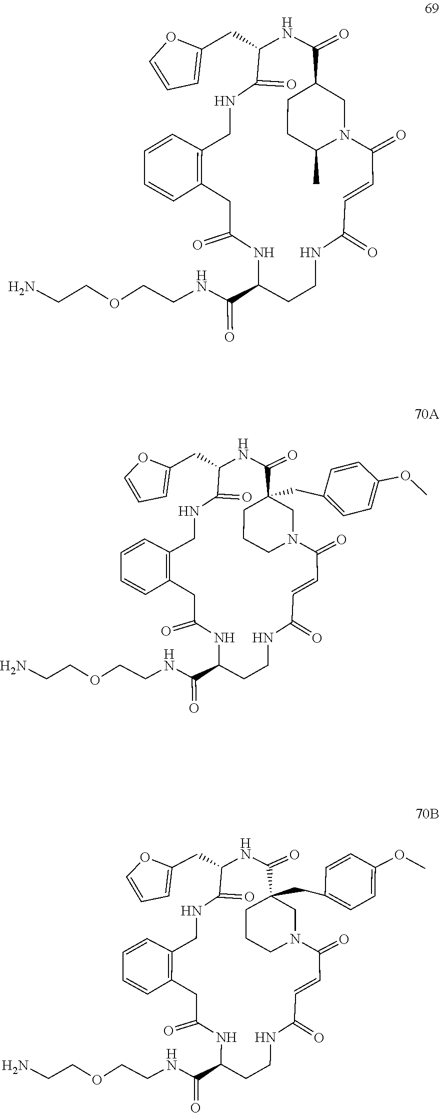 [SMILES: C[C@H]1CC[C@@H]2CN1C(=O)/C=C/C(=O)NCC[C@@H](C(=O)NCCOCCN)NC(=O)Cc1ccccc1CNC(=O)[C@H](Cc1ccco1)NC2=O ; COc1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; COc1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1]

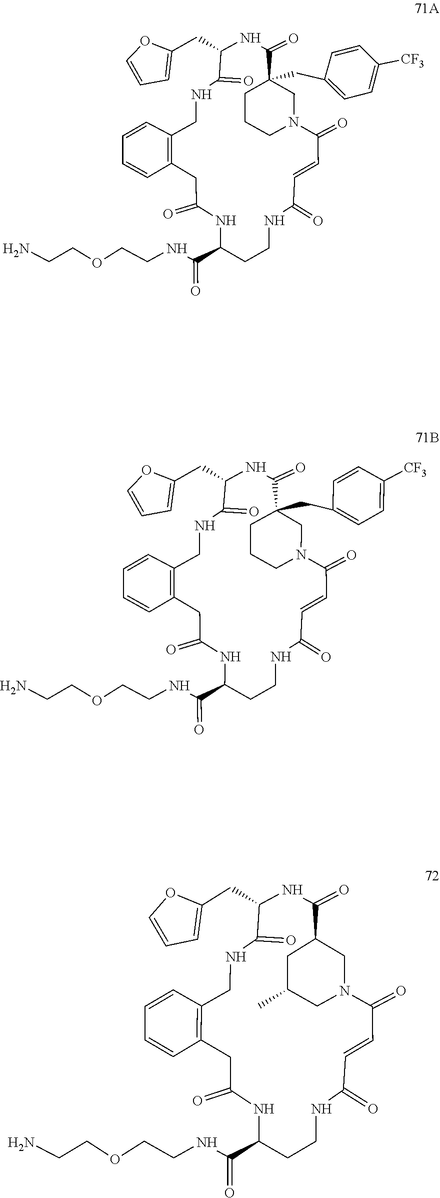 [SMILES: NCCOCCNC(=O)[C@@H]1CCNC(=O)/C=C/C(=O)N2CCC[C@](Cc3ccc(C(F)(F)F)cc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(C(F)(F)F)cc3)(C2)C(=O)N[C@@H](Cc2ccco2)C(=O)NCc2ccccc2CC(=O)N1 ; C[C@@H]1C[C@@H]2CN(C1)C(=O)/C=C/C(=O)NCC[C@@H](C(=O)NCCOCCN)NC(=O)Cc1ccccc1CNC(=O)[C@H](Cc1ccco1)NC2=O]

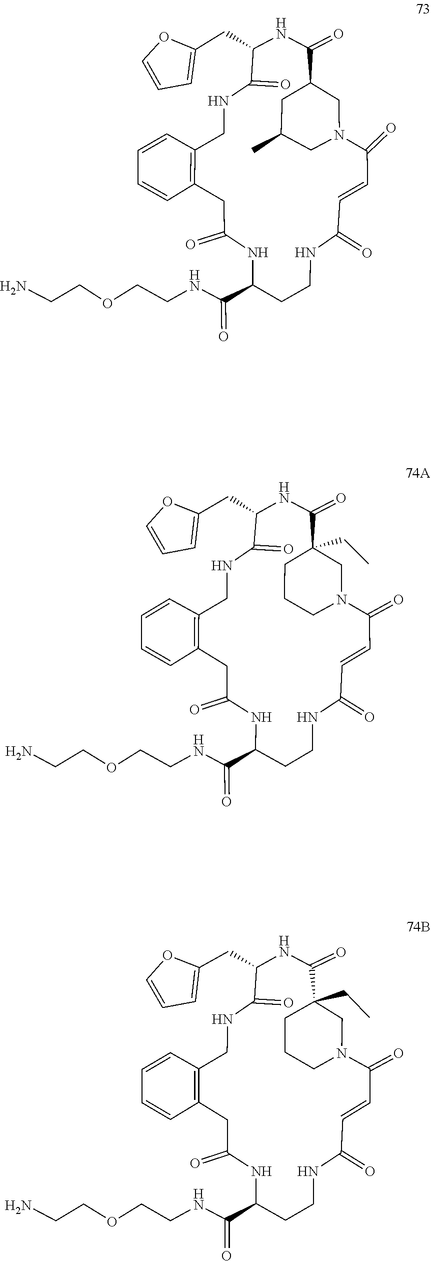 [SMILES: C[C@H]1C[C@@H]2CN(C1)C(=O)/C=C/C(=O)NCC[C@@H](C(=O)NCCOCCN)NC(=O)Cc1ccccc1CNC(=O)[C@H](Cc1ccco1)NC2=O ; CC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

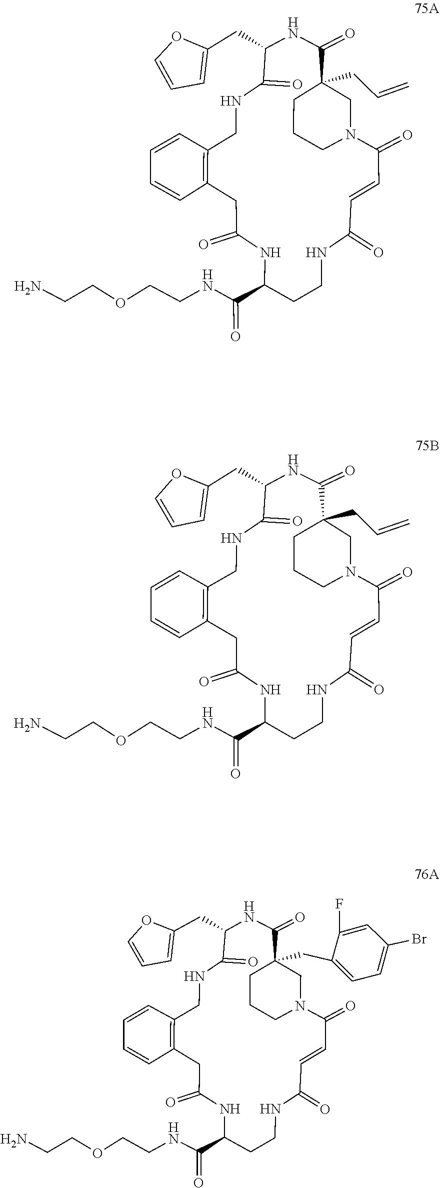 [SMILES: C=CC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; C=CC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; NCCOCCNC(=O)[C@@H]1CCNC(=O)/C=C/C(=O)N2CCC[C@](Cc3ccc(Br)cc3F)(C2)C(=O)N[C@@H](Cc2ccco2)C(=O)NCc2ccccc2CC(=O)N1]

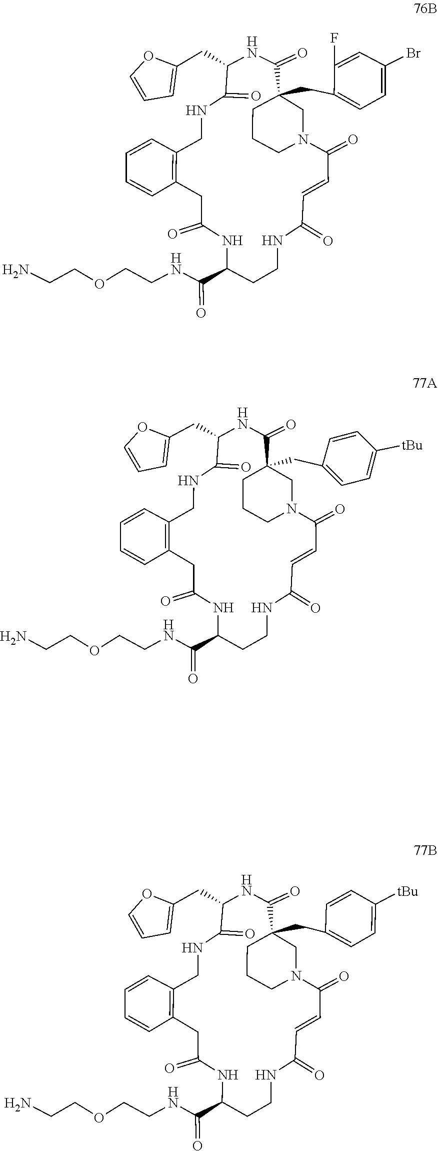 [SMILES: NCCOCCNC(=O)[C@@H]1CCNC(=O)/C=C/C(=O)N2CCC[C@@](Cc3ccc(Br)cc3F)(C2)C(=O)N[C@@H](Cc2ccco2)C(=O)NCc2ccccc2CC(=O)N1 ; CC(C)(C)c1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; CC(C)(C)c1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1]

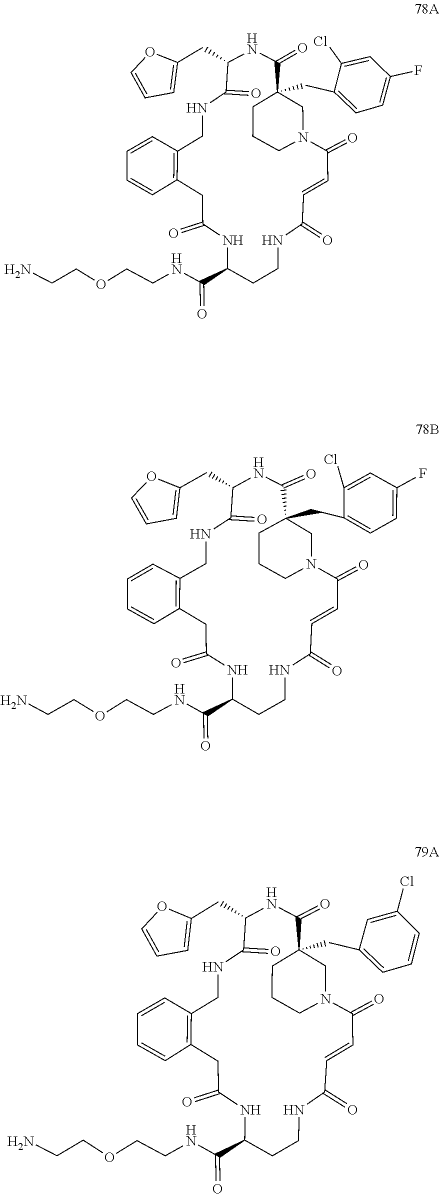 [SMILES: NCCOCCNC(=O)[C@@H]1CCNC(=O)/C=C/C(=O)N2CCC[C@](Cc3ccc(F)cc3Cl)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(F)cc3Cl)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3cccc(Cl)c3)(C2)C(=O)N[C@@H](Cc2ccco2)C(=O)NCc2ccccc2CC(=O)N1]

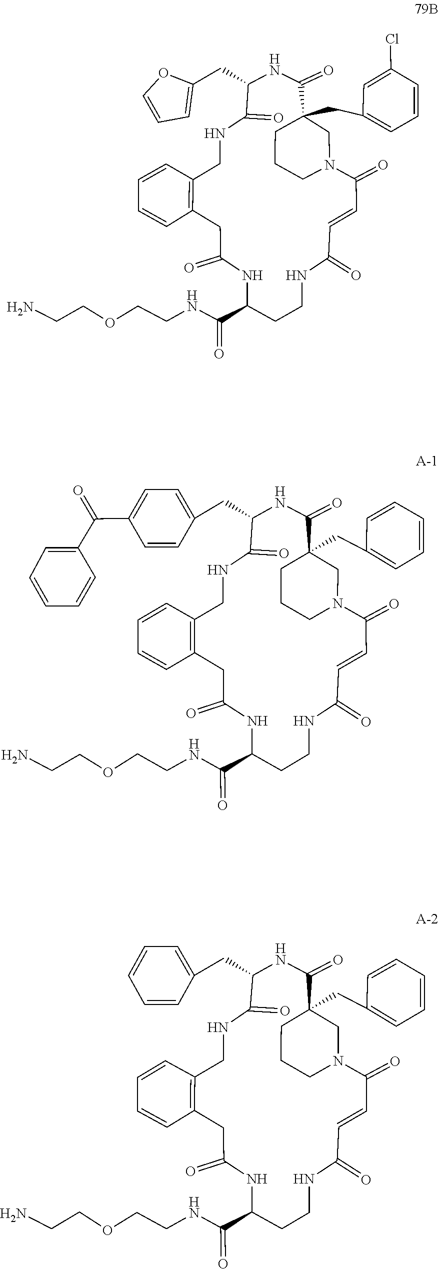 [SMILES: NCCOCCNC(=O)[C@@H]1CCNC(=O)/C=C/C(=O)N2CCC[C@@](Cc3cccc(Cl)c3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccccc2)C(=O)NCc2ccccc2CC(=O)N1]

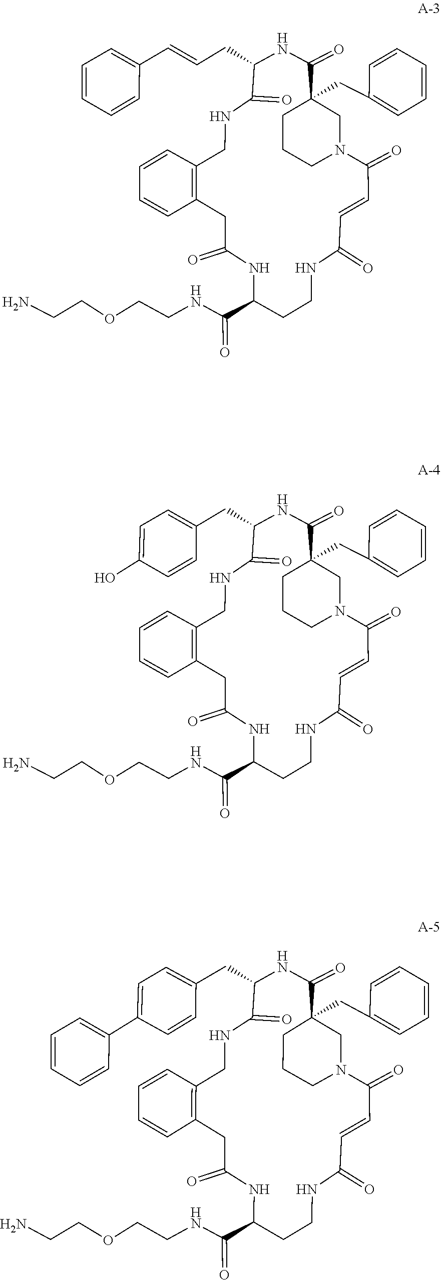 [SMILES: NCCOCCNC(=O)[C@@H]1CCNC(=O)/C=C/C(=O)N2CCC[C@](Cc3ccccc3)(C2)C(=O)N[C@@H](C/C=C/c2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(O)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccccc3)cc2)C(=O)NCc2ccccc2CC(=O)N1]

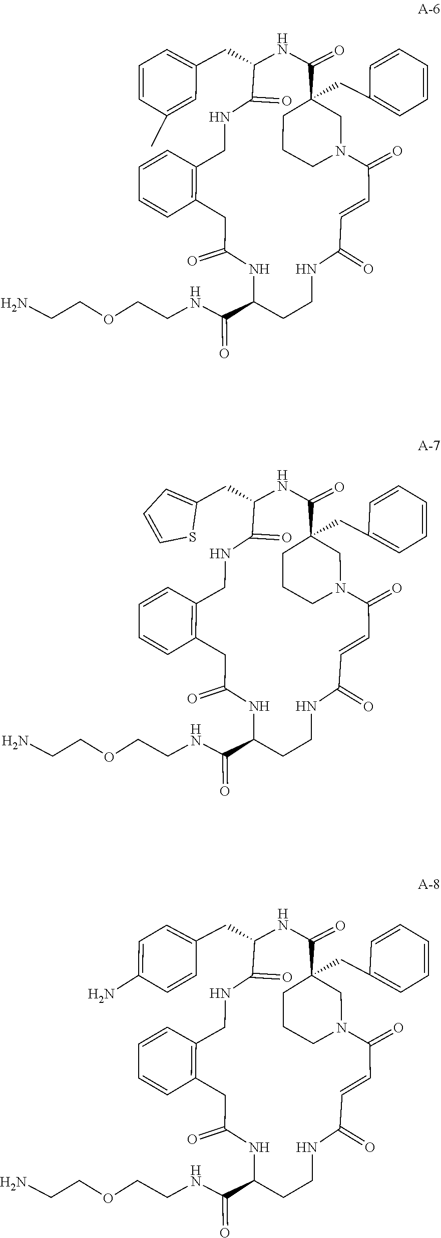 [SMILES: Cc1cccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)c1 ; NCCOCCNC(=O)[C@@H]1CCNC(=O)/C=C/C(=O)N2CCC[C@](Cc3ccccc3)(C2)C(=O)N[C@@H](Cc2cccs2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(N)cc2)C(=O)NCc2ccccc2CC(=O)N1]

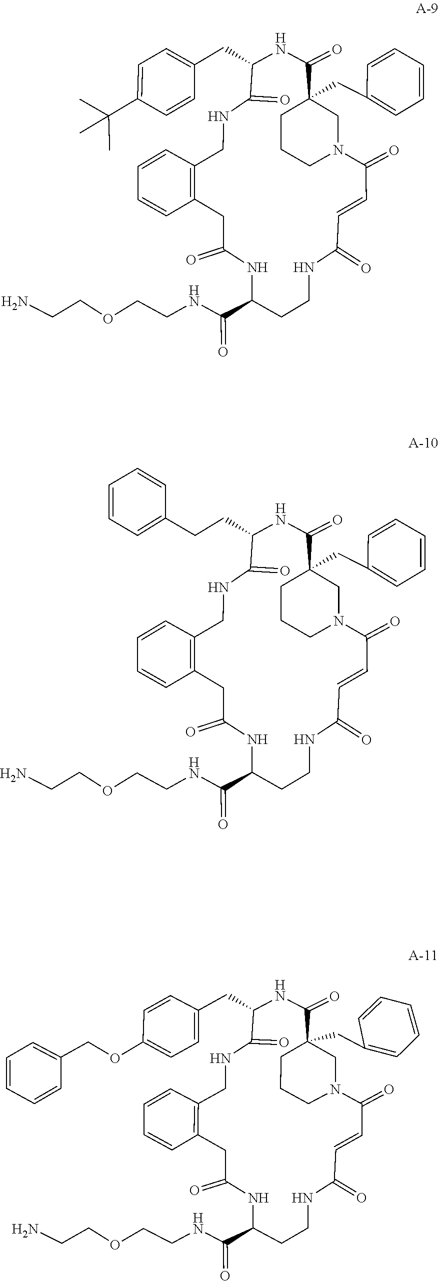 [SMILES: CC(C)(C)c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](CCc2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(OCc3ccccc3)cc2)C(=O)NCc2ccccc2CC(=O)N1]

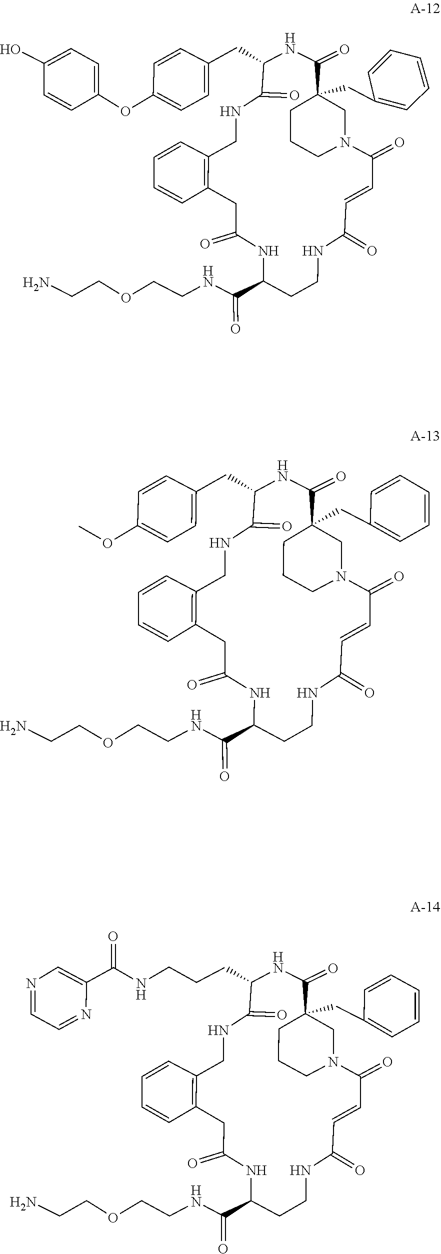 [SMILES: NCCOCCNC(=O)[C@@H]1CCNC(=O)/C=C/C(=O)N2CCC[C@](Cc3ccccc3)(C2)C(=O)N[C@@H](Cc2ccc(Oc3ccc(O)cc3)cc2)C(=O)NCc2ccccc2CC(=O)N1 ; COc1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](CCCNC(=O)c2cnccn2)C(=O)NCc2ccccc2CC(=O)N1]

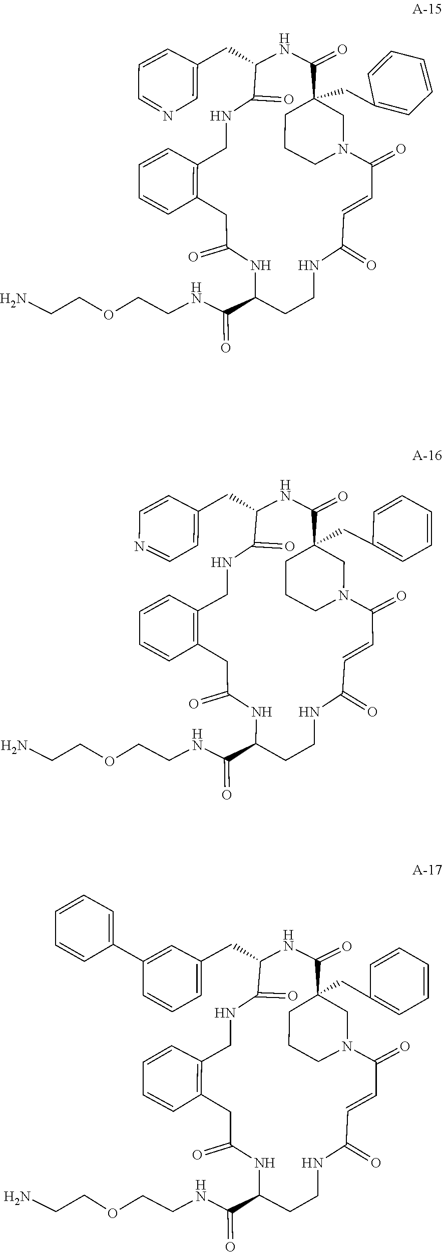 [SMILES: NCCOCCNC(=O)[C@@H]1CCNC(=O)/C=C/C(=O)N2CCC[C@](Cc3ccccc3)(C2)C(=O)N[C@@H](Cc2cccnc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccncc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccc(-c3ccccc3)c2)C(=O)NCc2ccccc2CC(=O)N1]

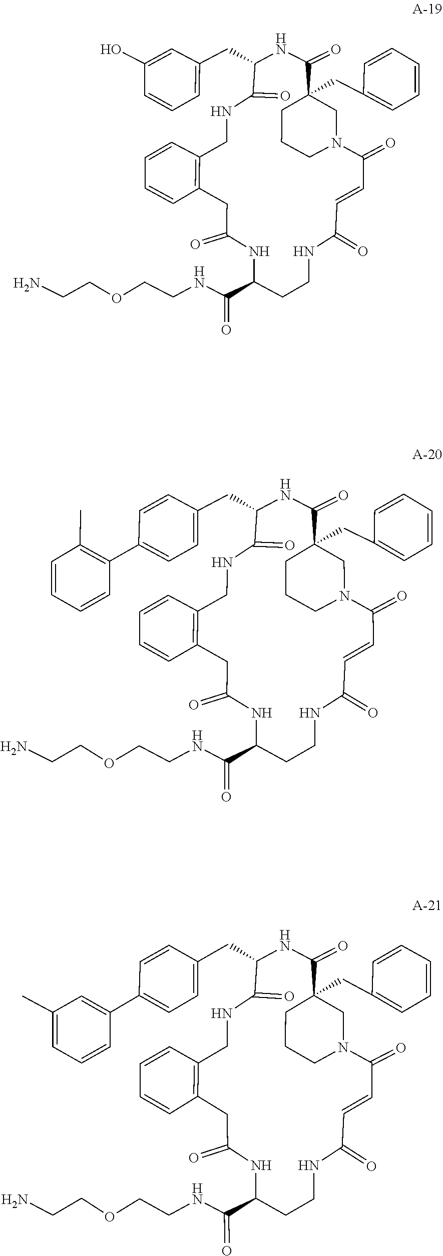 [SMILES: NCCOCCNC(=O)[C@@H]1CCNC(=O)/C=C/C(=O)N2CCC[C@](Cc3ccccc3)(C2)C(=O)N[C@@H](Cc2cccc(O)c2)C(=O)NCc2ccccc2CC(=O)N1 ; Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; Cc1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1]

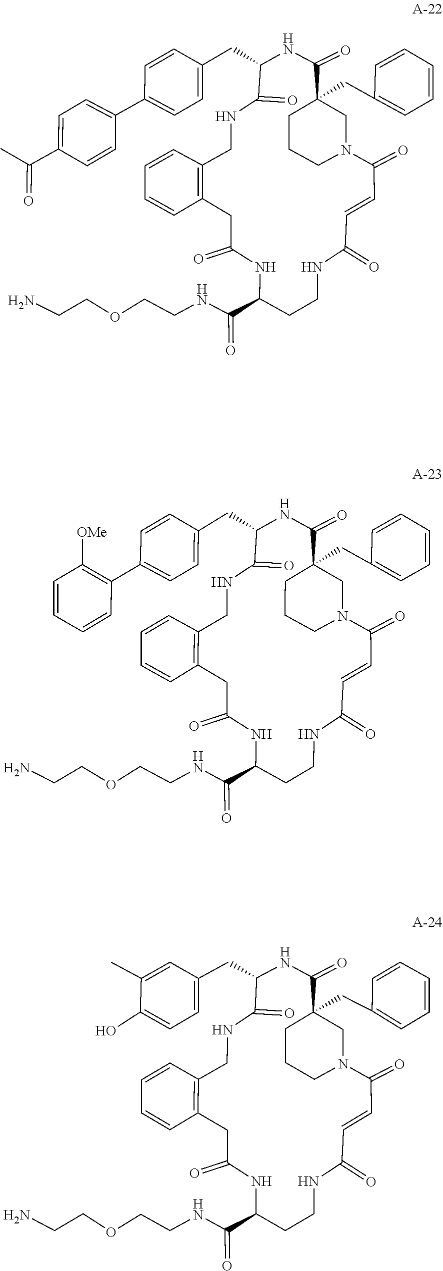 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; COc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; Cc1cc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)ccc1O]

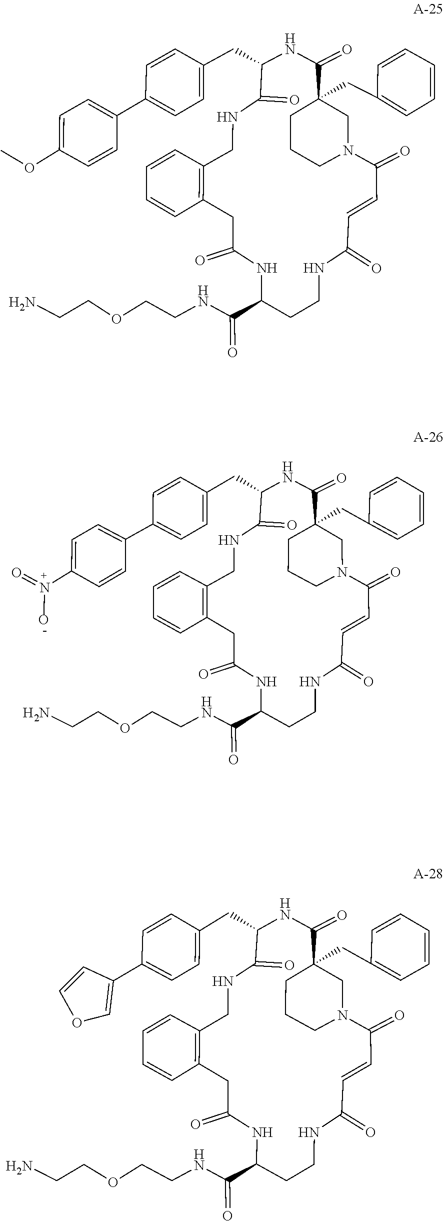

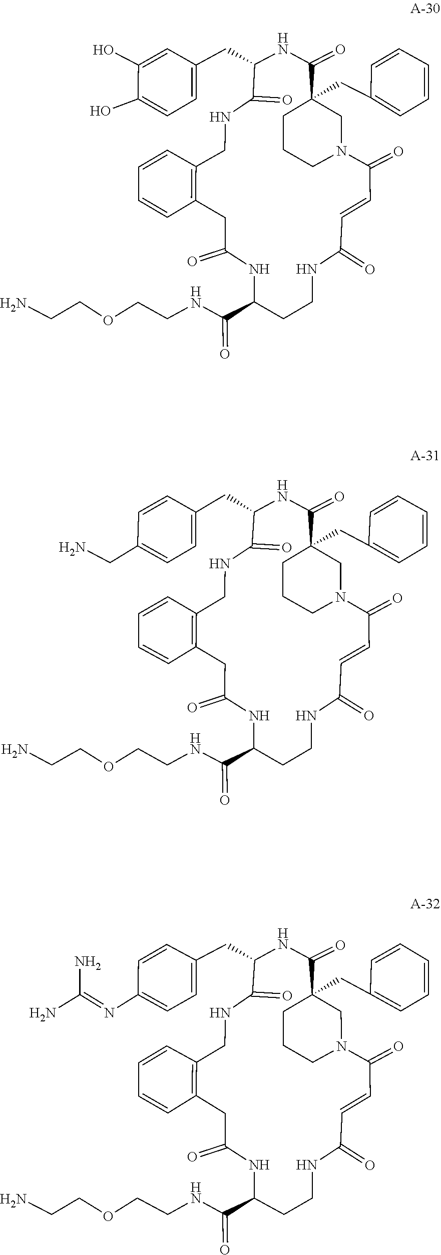 [SMILES: NCCOCCNC(=O)[C@@H]1CCNC(=O)/C=C/C(=O)N2CCC[C@](Cc3ccccc3)(C2)C(=O)N[C@@H](Cc2ccc(O)c(O)c2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(CN)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(N=C(N)N)cc2)C(=O)NCc2ccccc2CC(=O)N1]

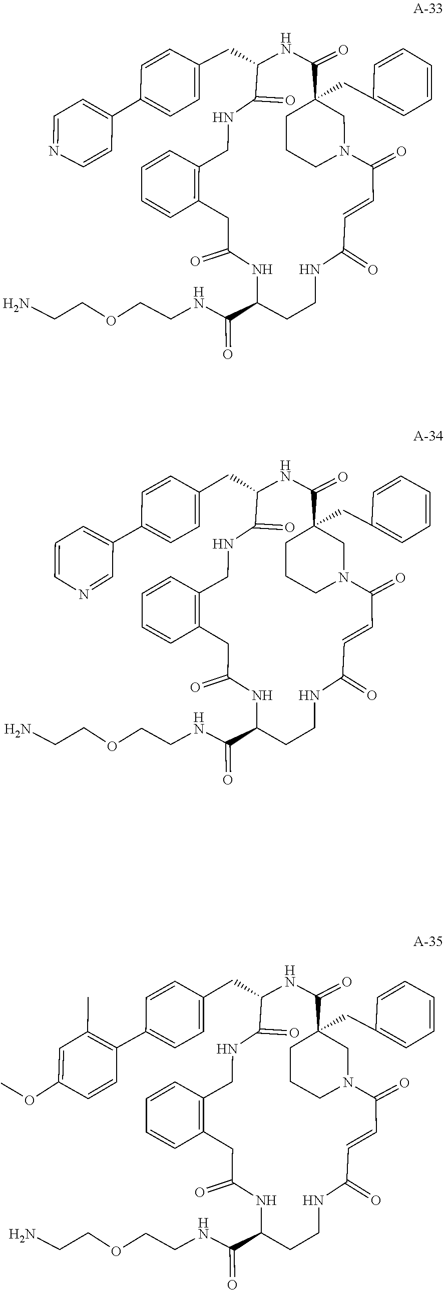 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccncc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3cccnc3)cc2)C(=O)NCc2ccccc2CC(=O)N1 ; COc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(C)c1]

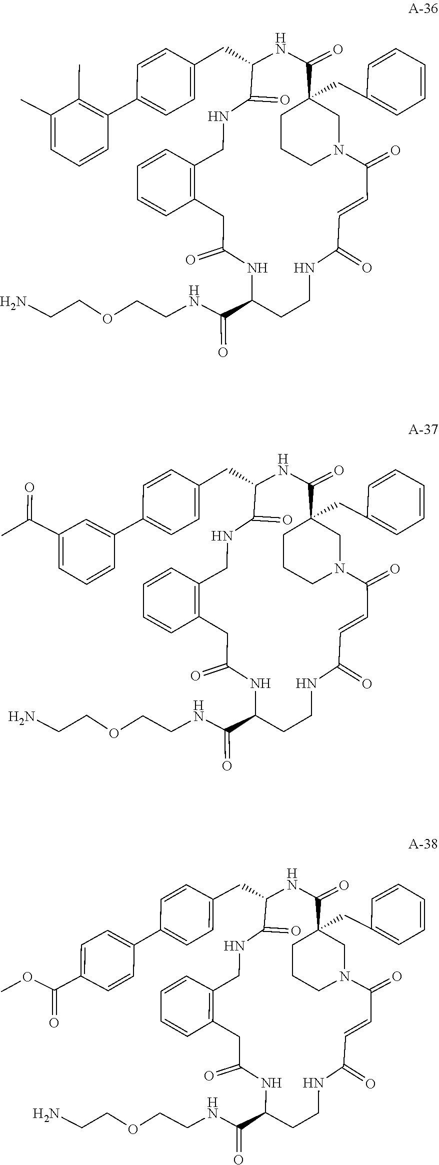 [SMILES: Cc1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1C ; CC(=O)c1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1 ; COC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

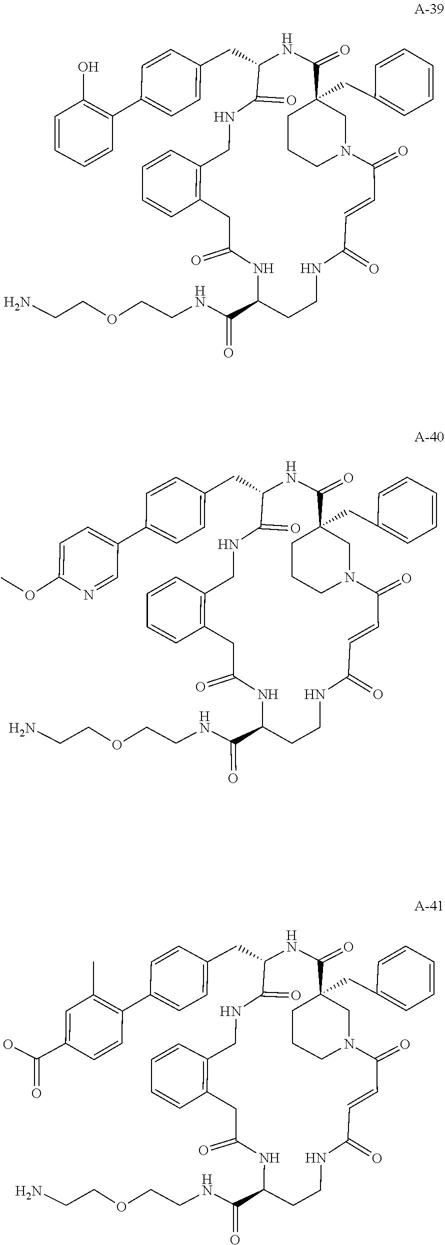

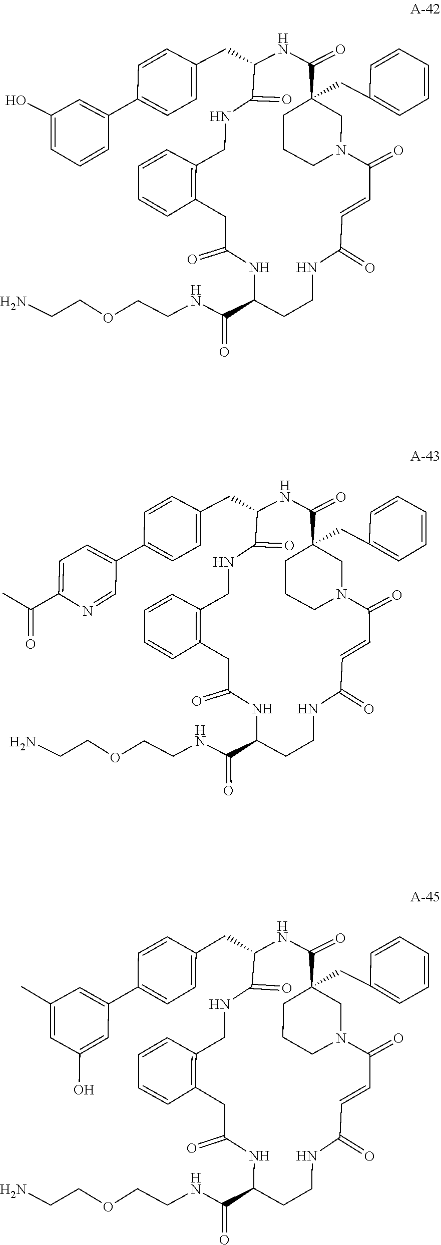 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3cccc(O)c3)cc2)C(=O)NCc2ccccc2CC(=O)N1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cn1 ; Cc1cc(O)cc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1]

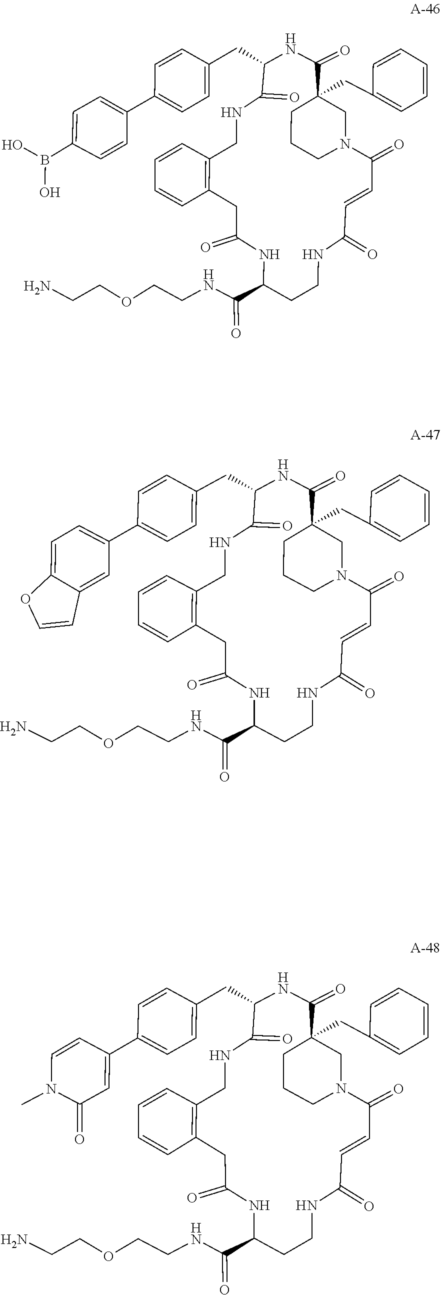 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(B(O)O)cc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc4occc4c3)cc2)C(=O)NCc2ccccc2CC(=O)N1 ; Cn1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1=O]

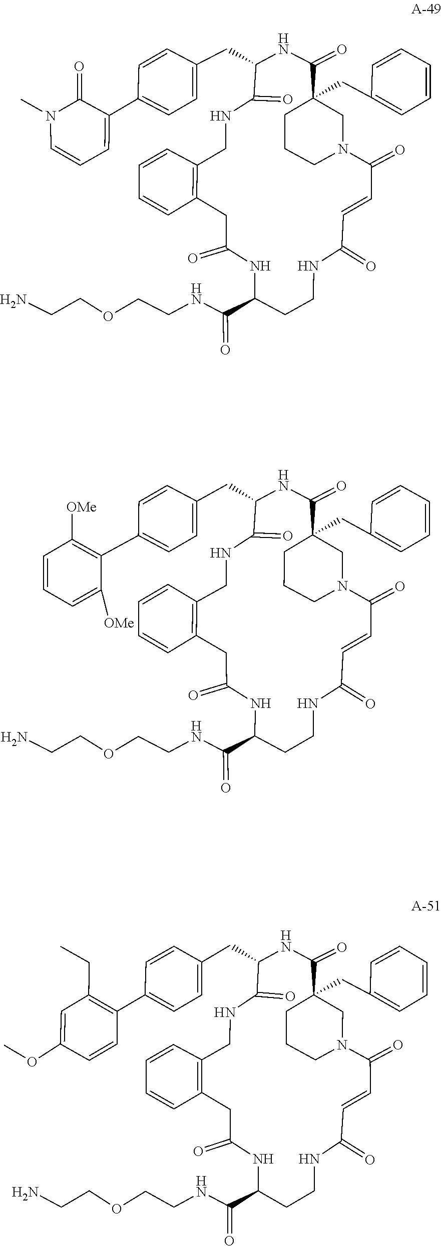 [SMILES: Cn1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1=O ; COc1cccc(OC)c1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; CCc1cc(OC)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1]

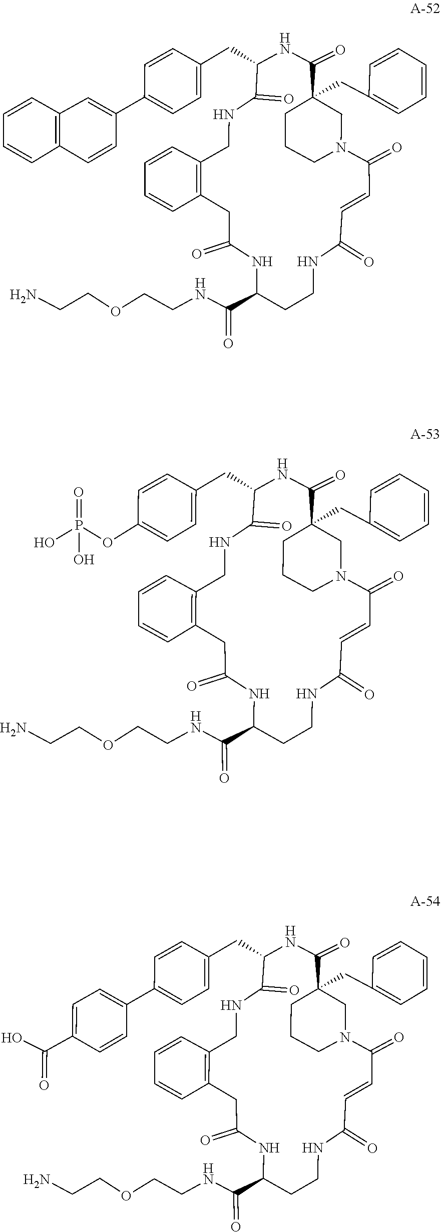 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc4ccccc4c3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(OP(=O)(O)O)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(C(=O)O)cc3)cc2)C(=O)NCc2ccccc2CC(=O)N1]

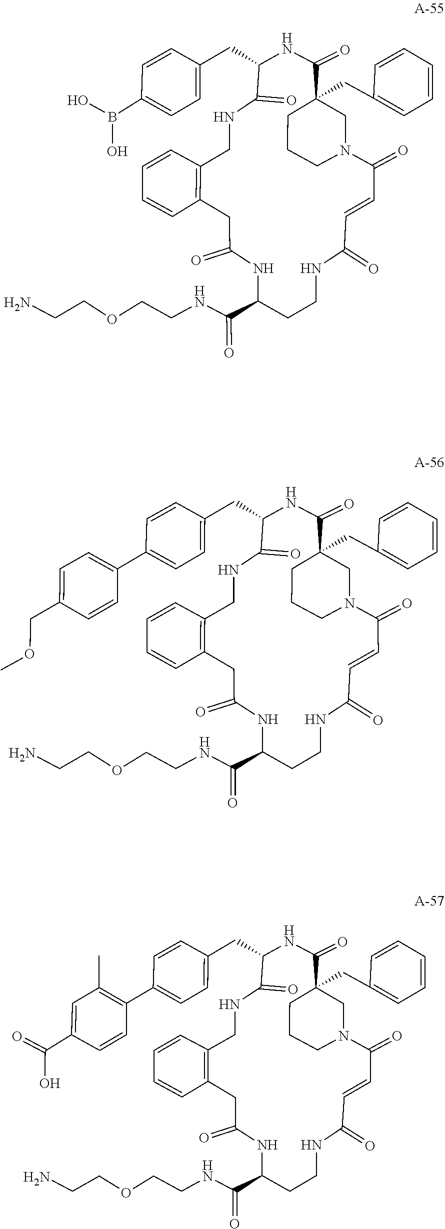 [SMILES: NCCOCCNC(=O)[C@@H]1CCNC(=O)/C=C/C(=O)N2CCC[C@](Cc3ccccc3)(C2)C(=O)N[C@@H](Cc2ccc(B(O)O)cc2)C(=O)NCc2ccccc2CC(=O)N1 ; COCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; Cc1cc(C(=O)O)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1]

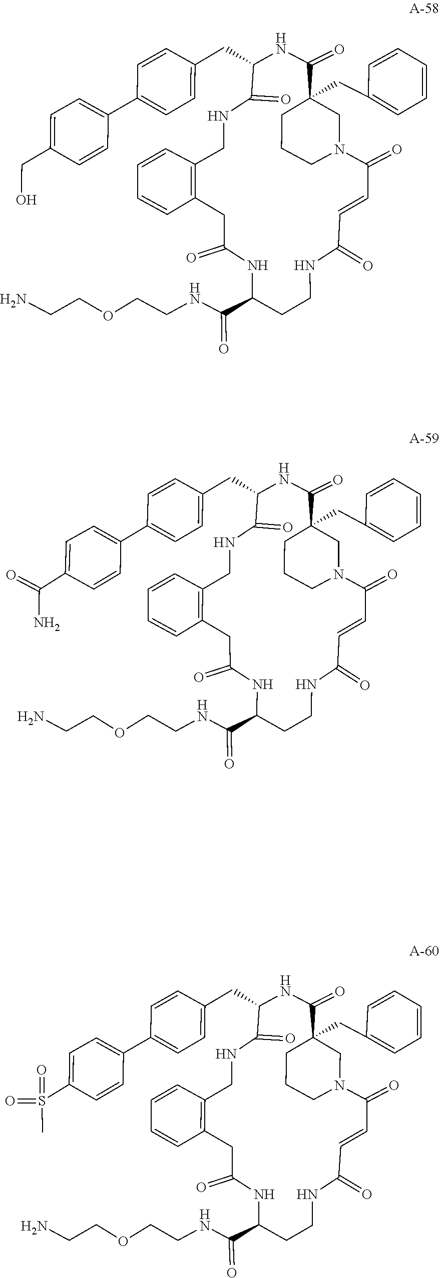 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(CO)cc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(C(N)=O)cc3)cc2)C(=O)NCc2ccccc2CC(=O)N1 ; CS(=O)(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

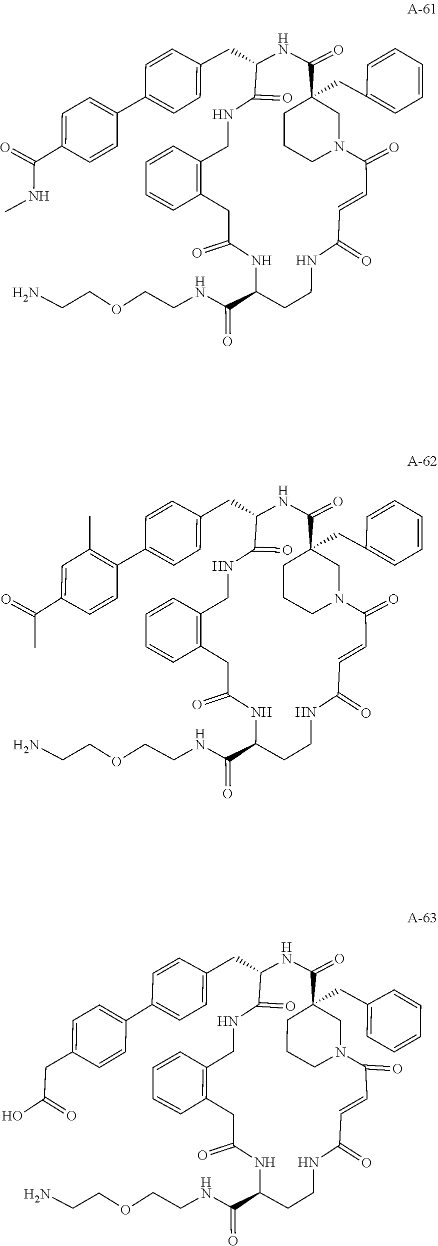 [SMILES: CNC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(C)c1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CC(=O)O)cc3)cc2)C(=O)NCc2ccccc2CC(=O)N1]

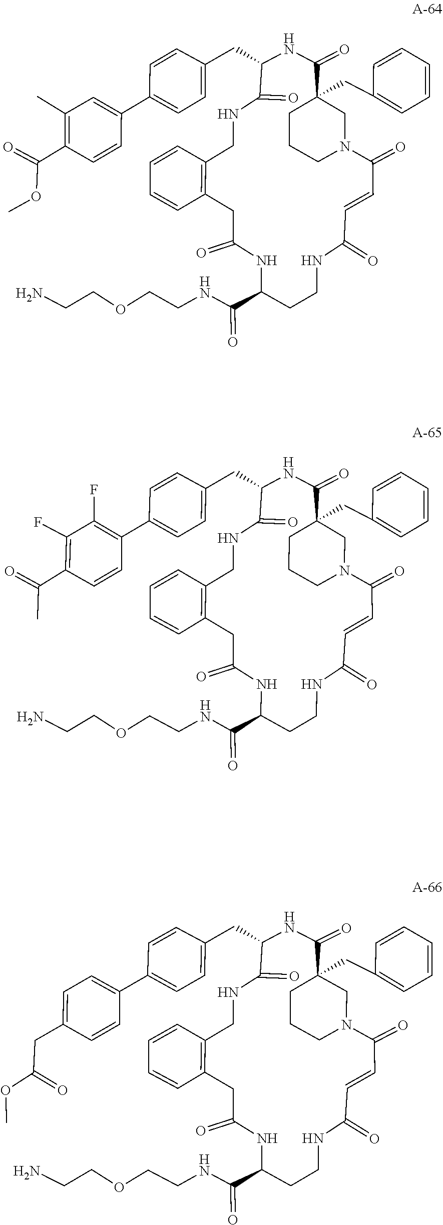 [SMILES: COC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1C ; CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(F)c1F ; COC(=O)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

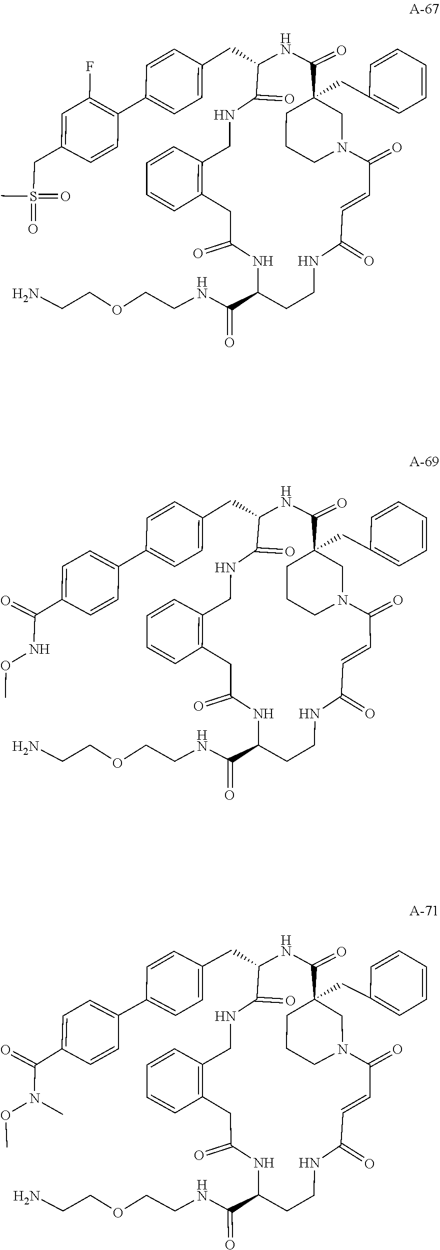 [SMILES: CS(=O)(=O)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(F)c1 ; CONC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CON(C)C(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

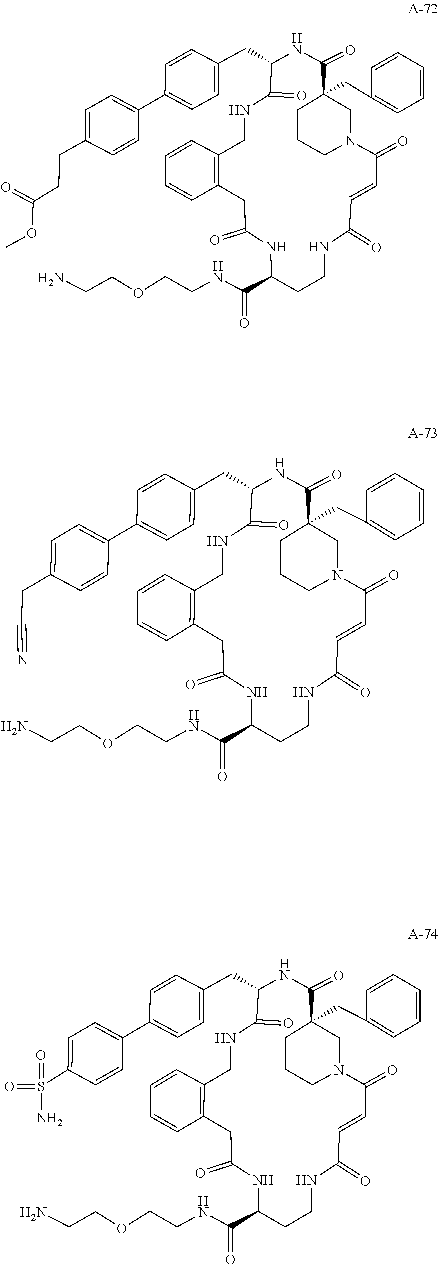 [SMILES: COC(=O)CCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; N#CCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(S(N)(=O)=O)cc3)cc2)C(=O)NCc2ccccc2CC(=O)N1]

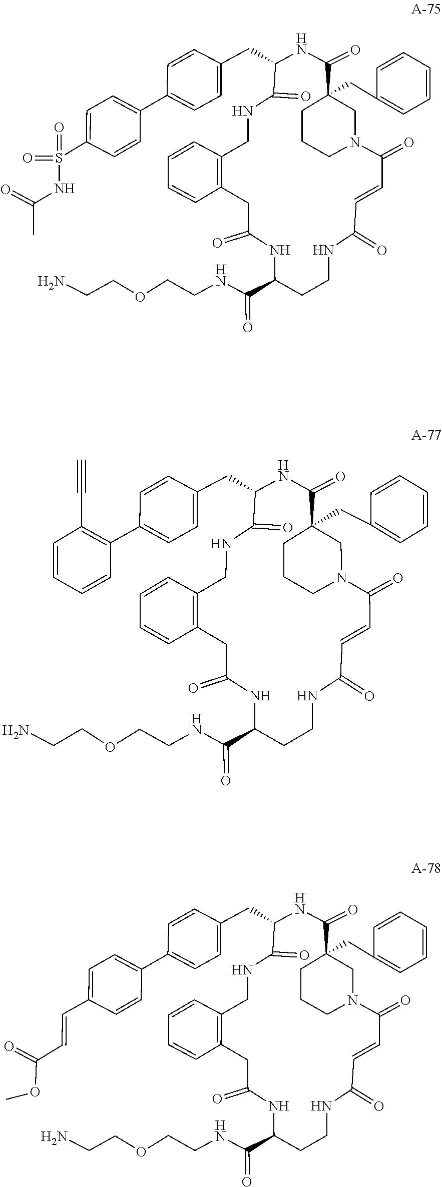 [SMILES: CC(=O)NS(=O)(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; C#Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; COC(=O)/C=C/c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

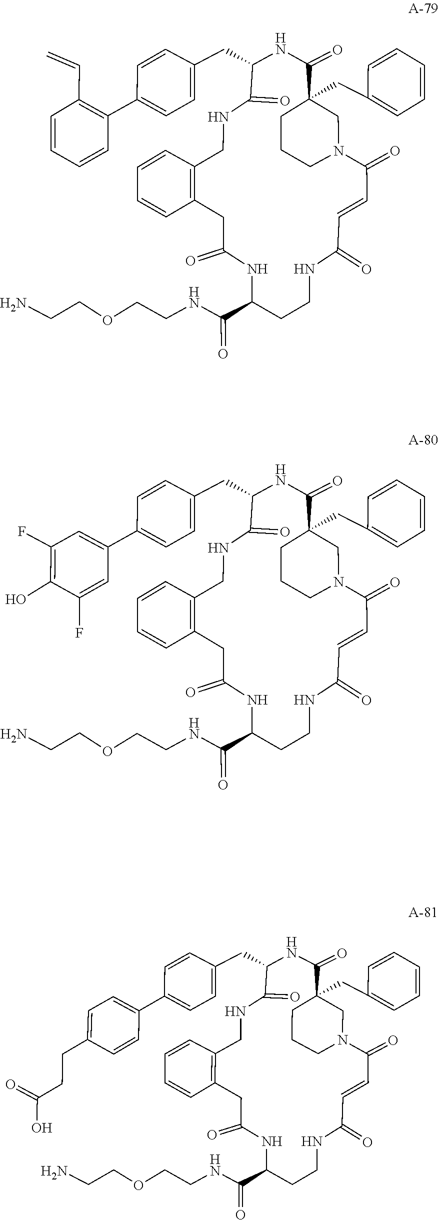 [SMILES: C=Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3cc(F)c(O)c(F)c3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCC(=O)O)cc3)cc2)C(=O)NCc2ccccc2CC(=O)N1]

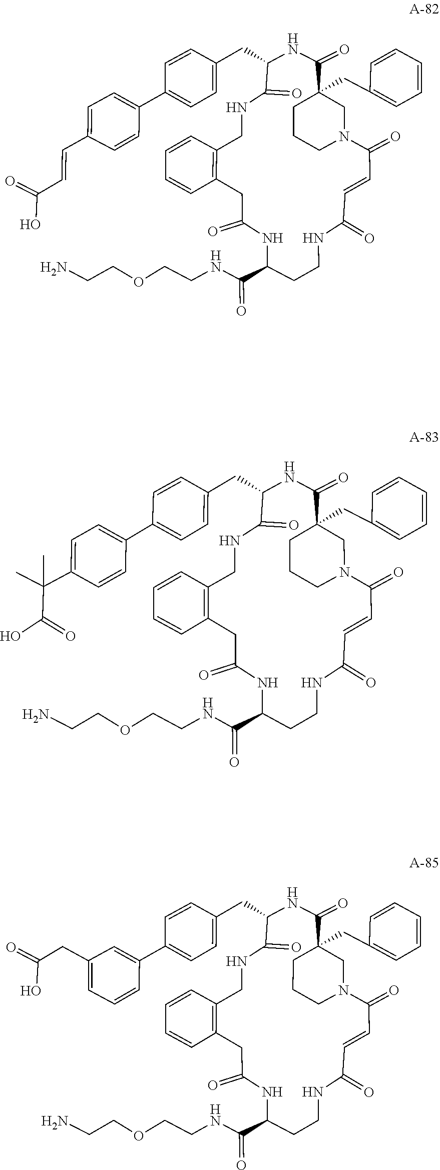 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(/C=C/C(=O)O)cc3)cc2)C(=O)NCc2ccccc2CC(=O)N1 ; CC(C)(C(=O)O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3cccc(CC(=O)O)c3)cc2)C(=O)NCc2ccccc2CC(=O)N1]

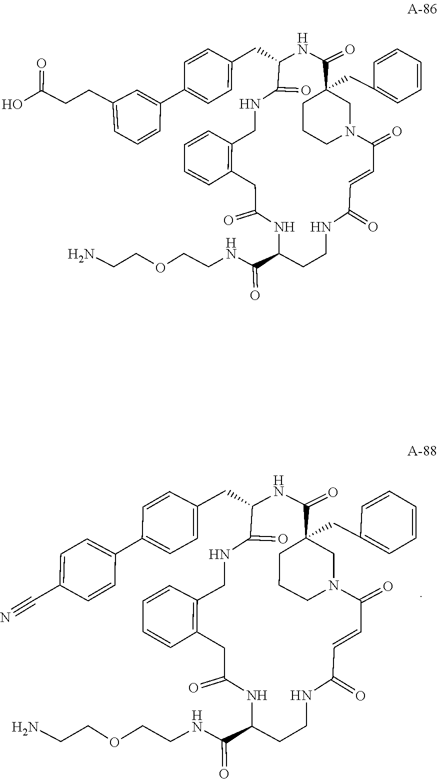 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3cccc(CCC(=O)O)c3)cc2)C(=O)NCc2ccccc2CC(=O)N1 ; N#Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

and pharmaceutically acceptable salts, solvates, hydrates, polymorphs, co-crystals, tautomers, stereoisomers, isotopically labeled derivatives, and prodrugs thereof.

Exemplary compounds of Formula (I′) or (I) include, but are not limited to:

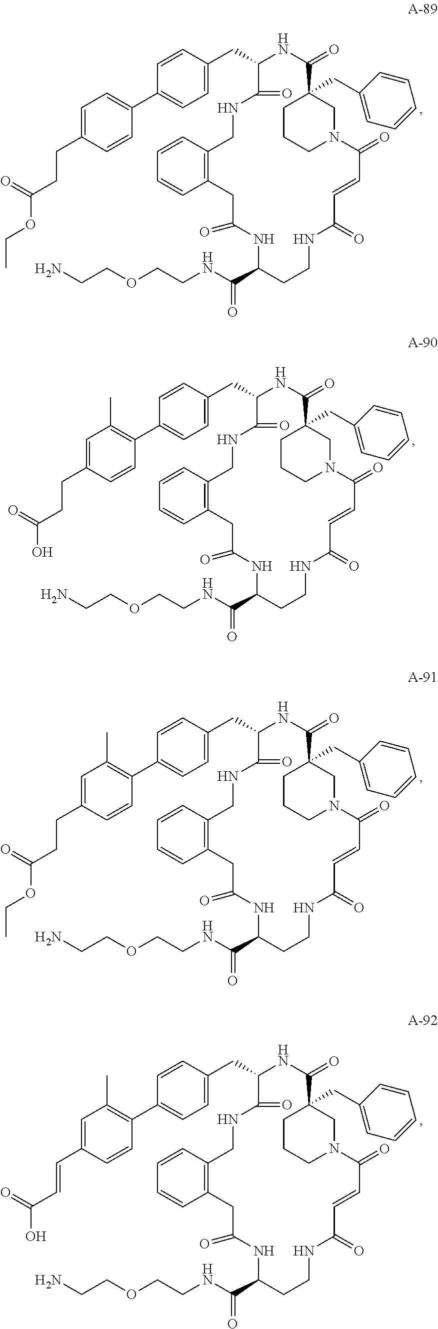

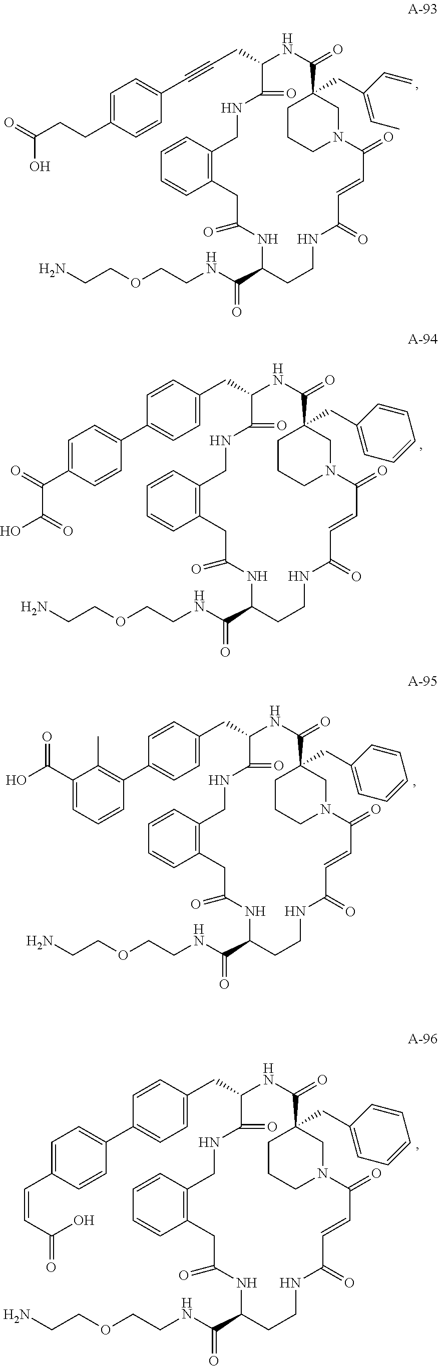

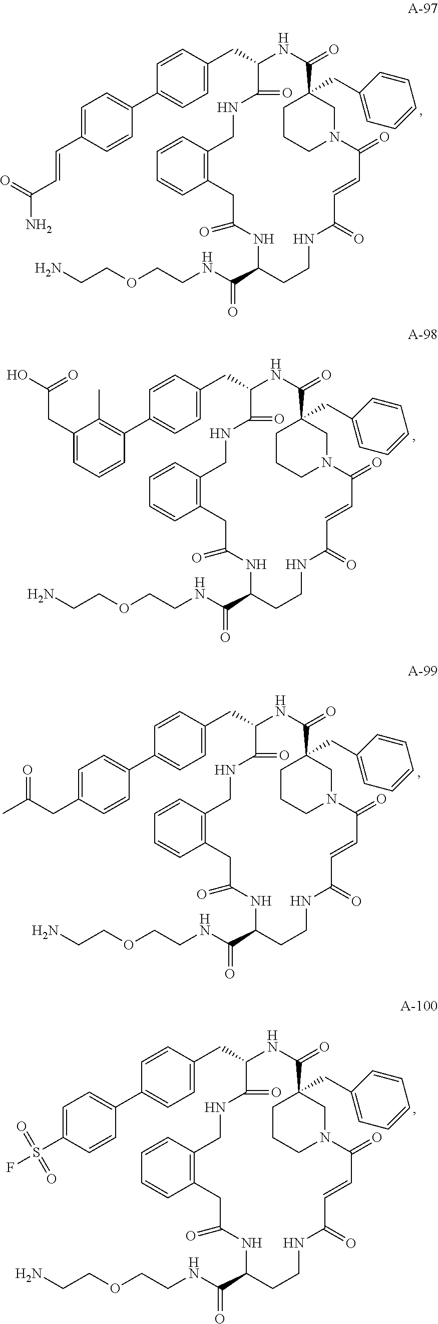

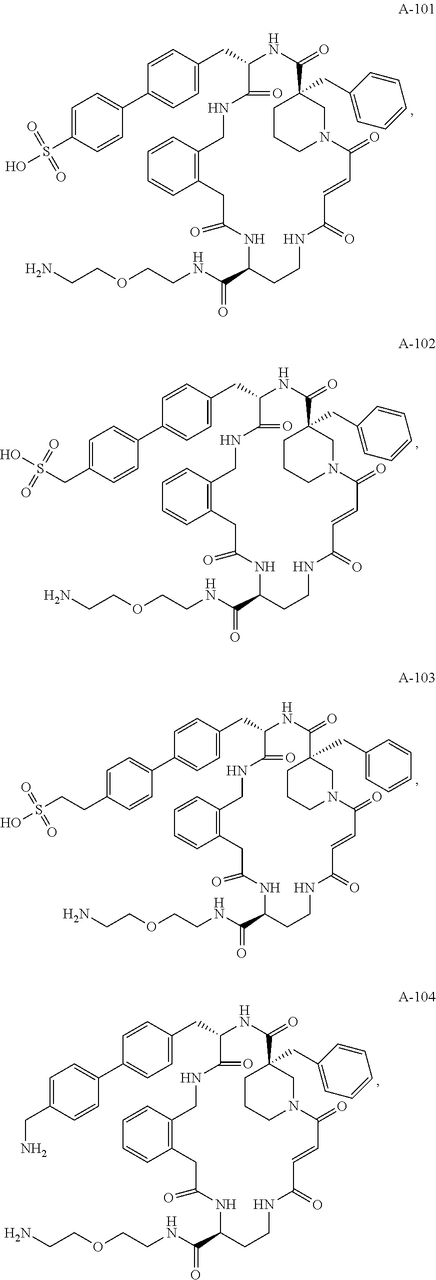

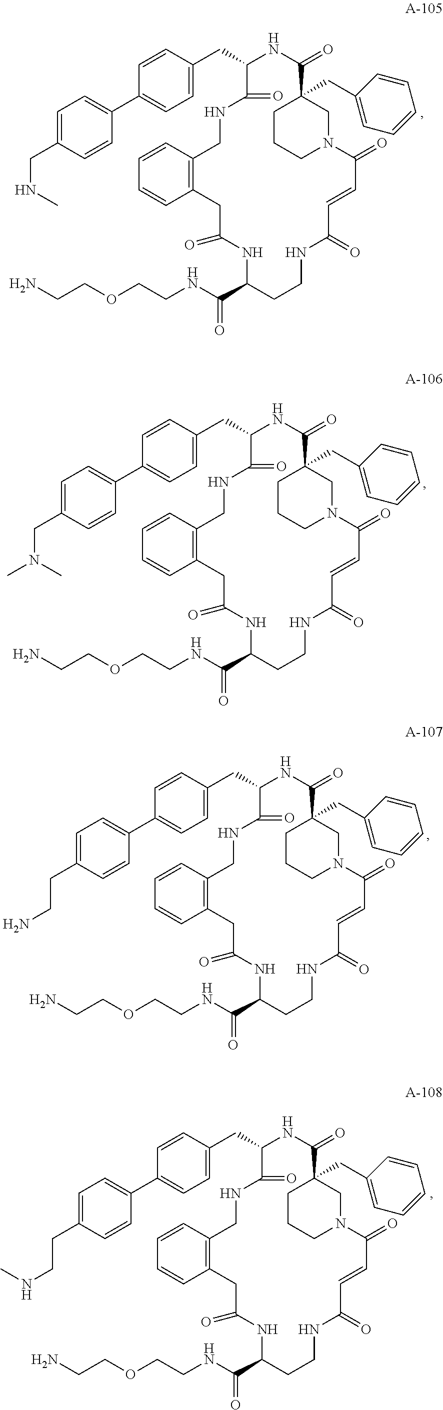 [SMILES: CNCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CN(C)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCN)cc3)cc2)C(=O)NCc2ccccc2CC(=O)N1 ; CNCCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

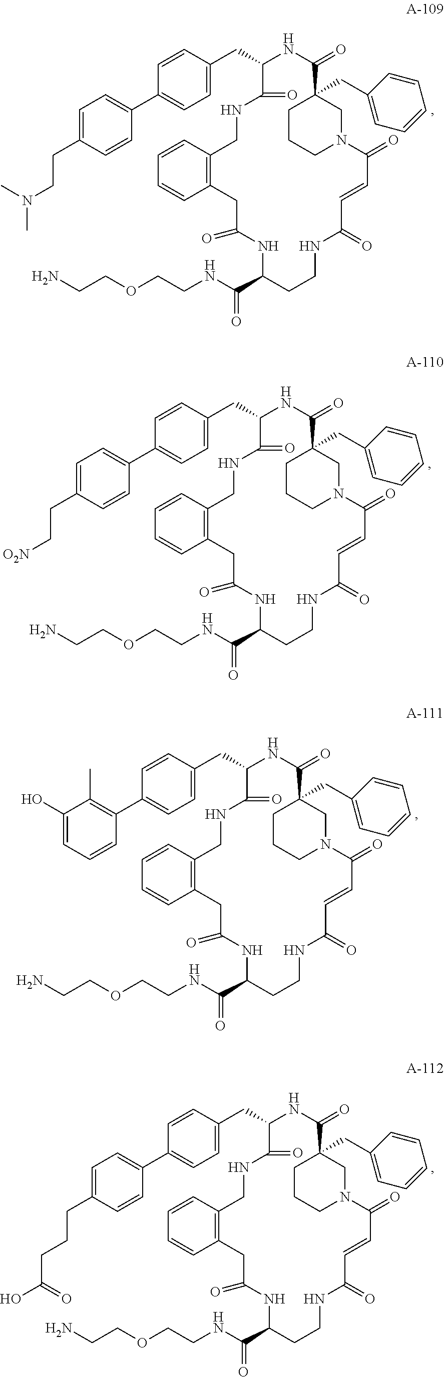

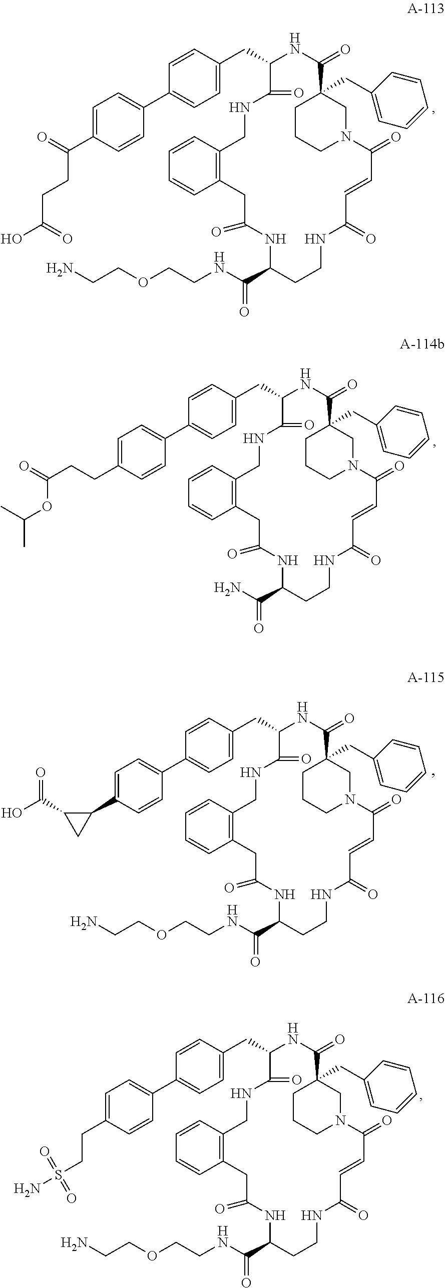

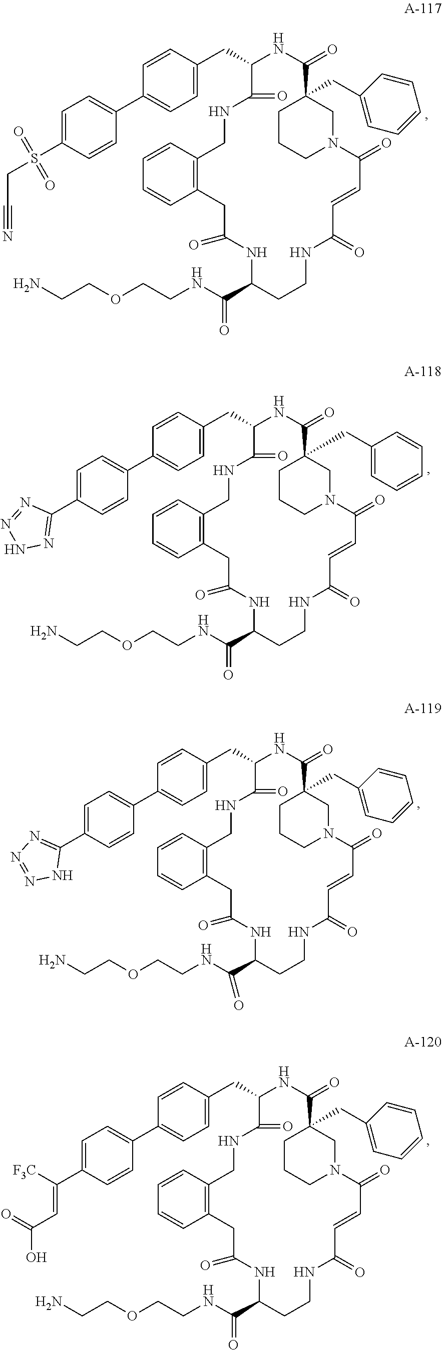

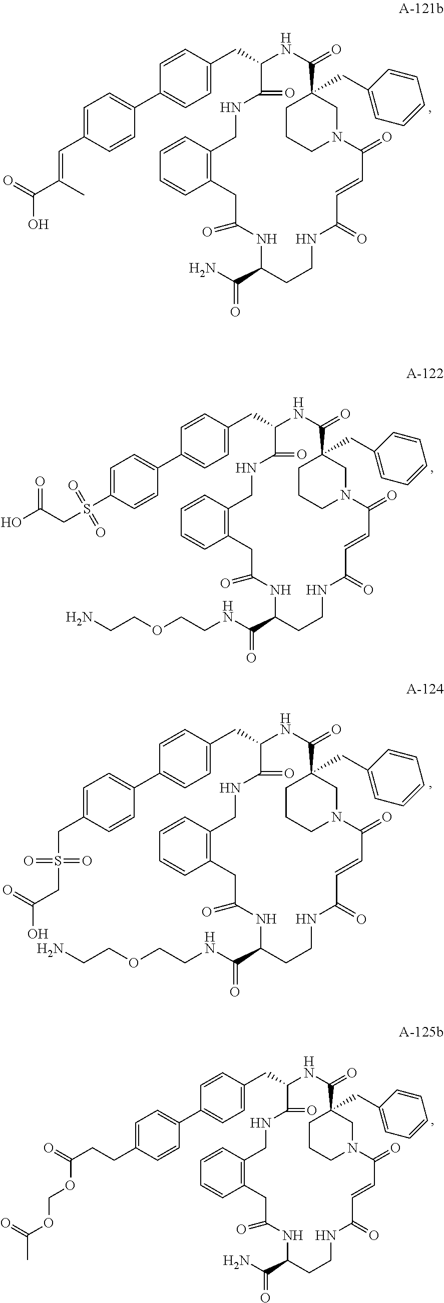

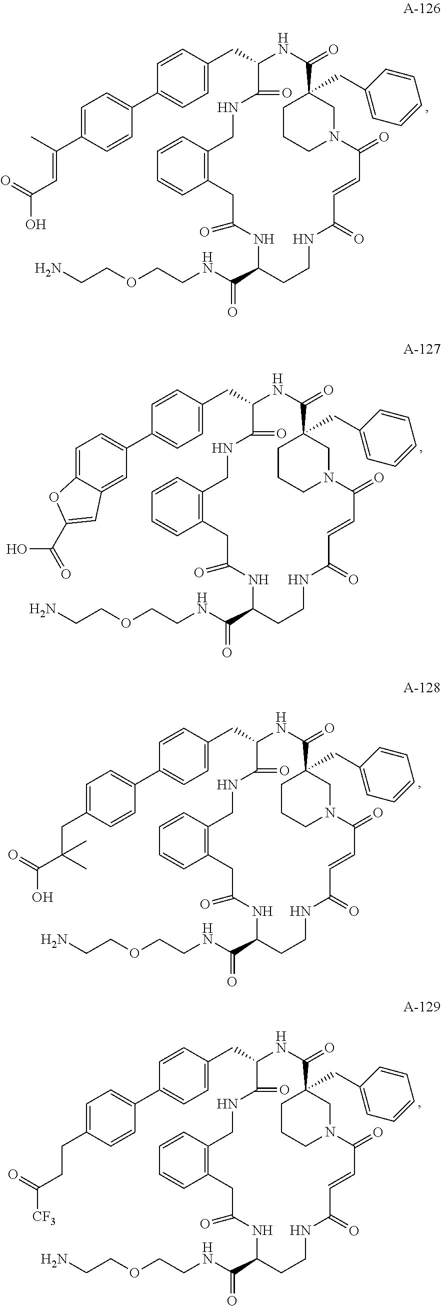

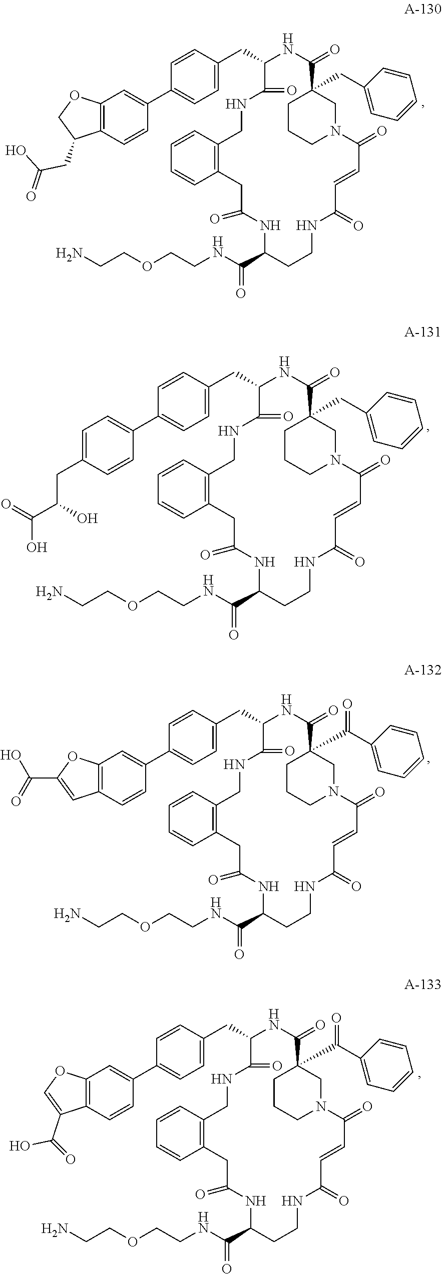

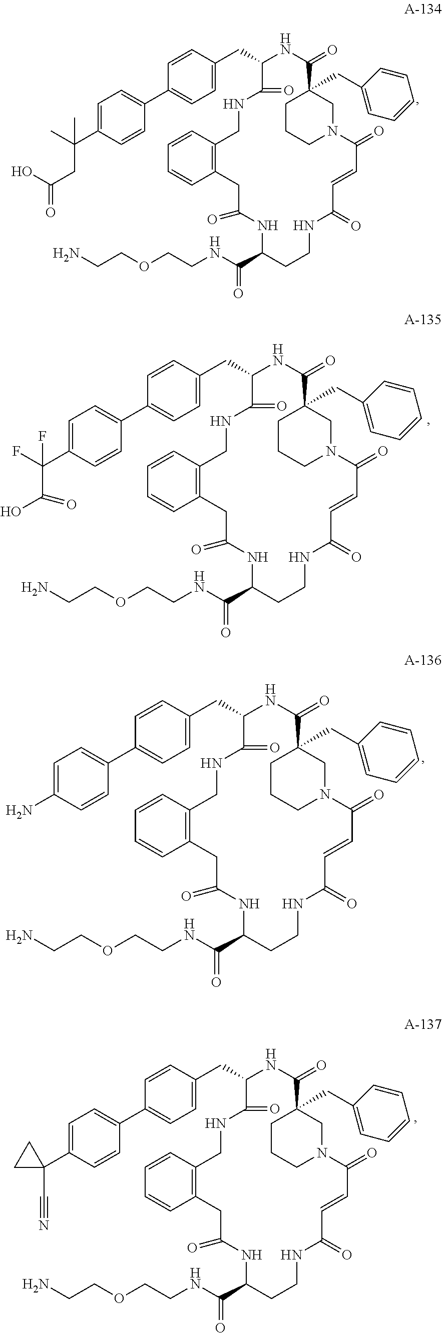

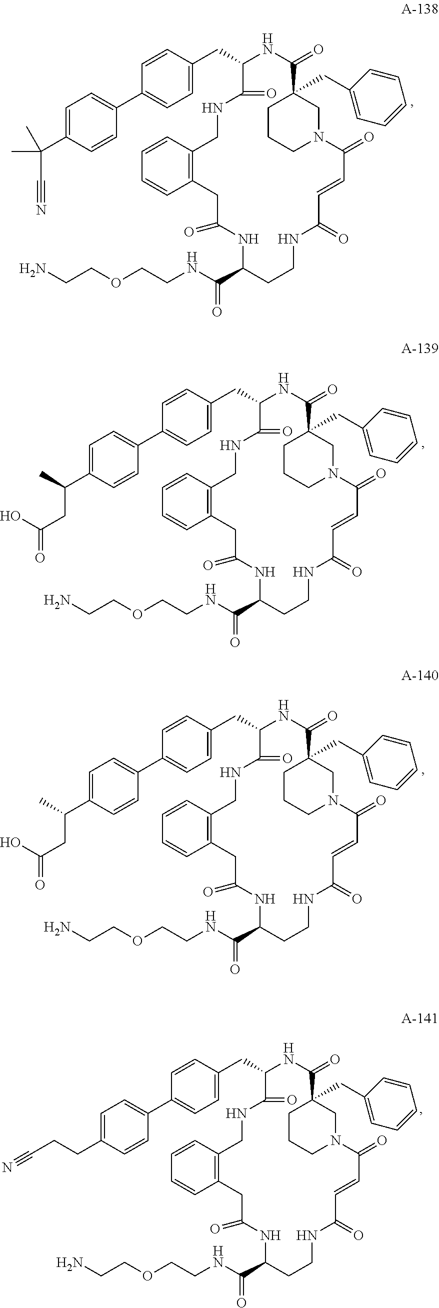

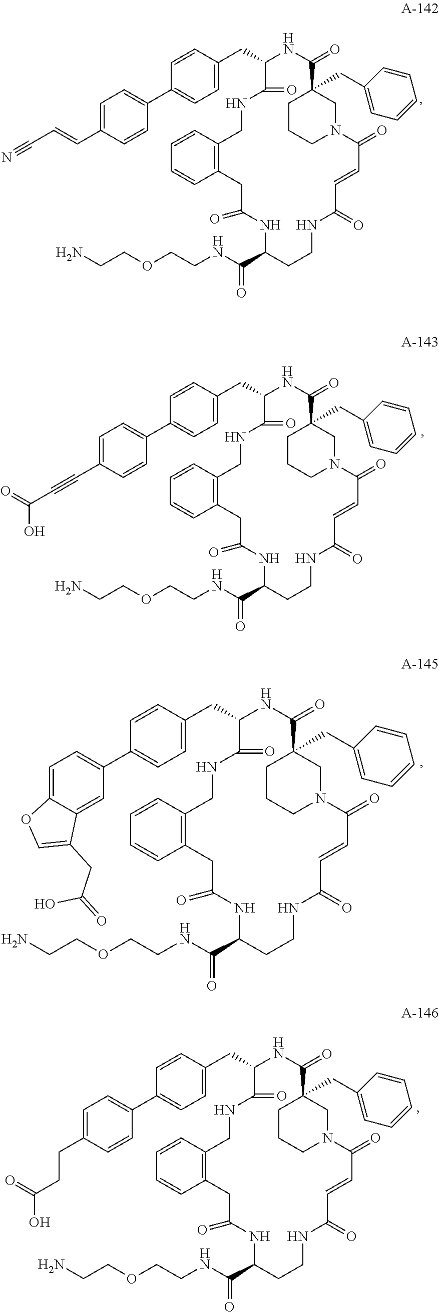

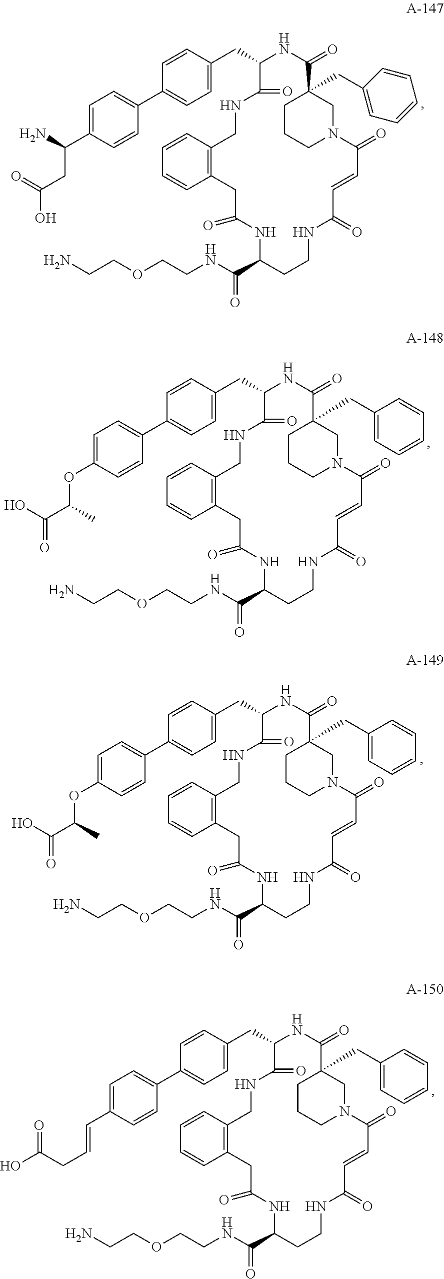

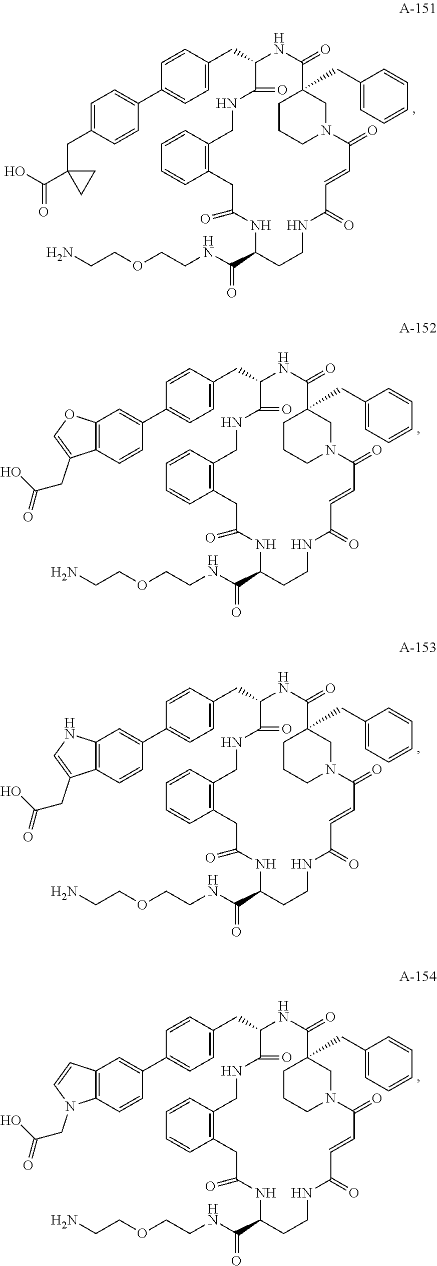

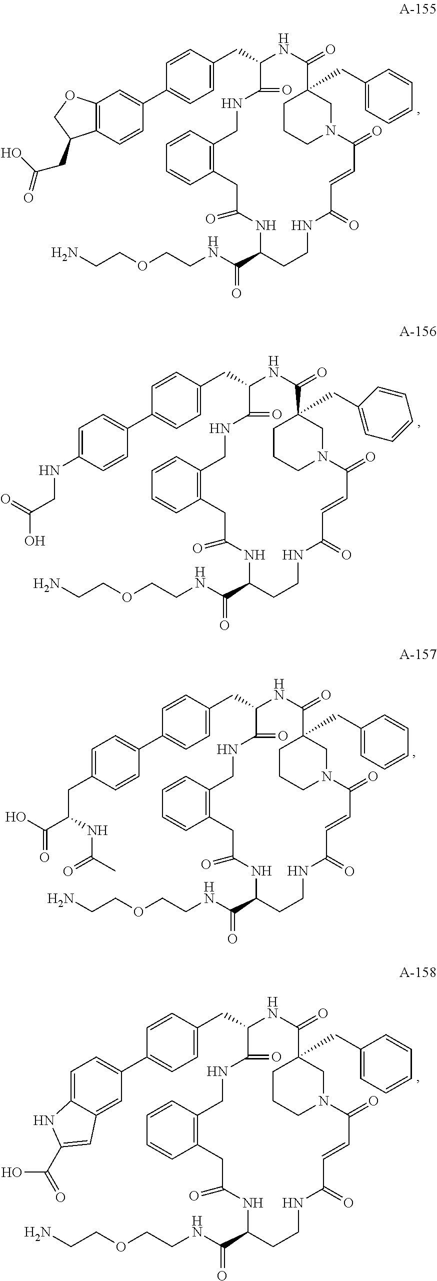

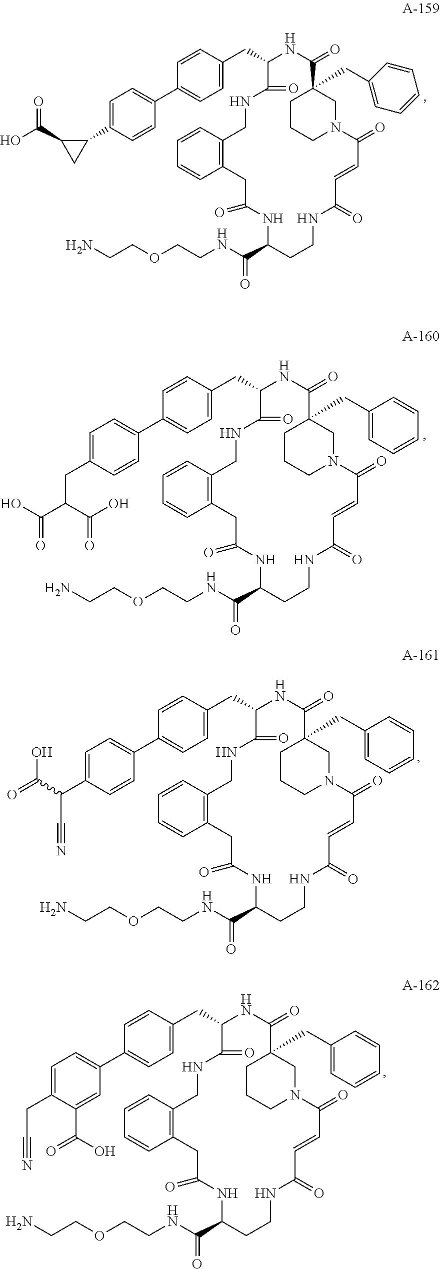

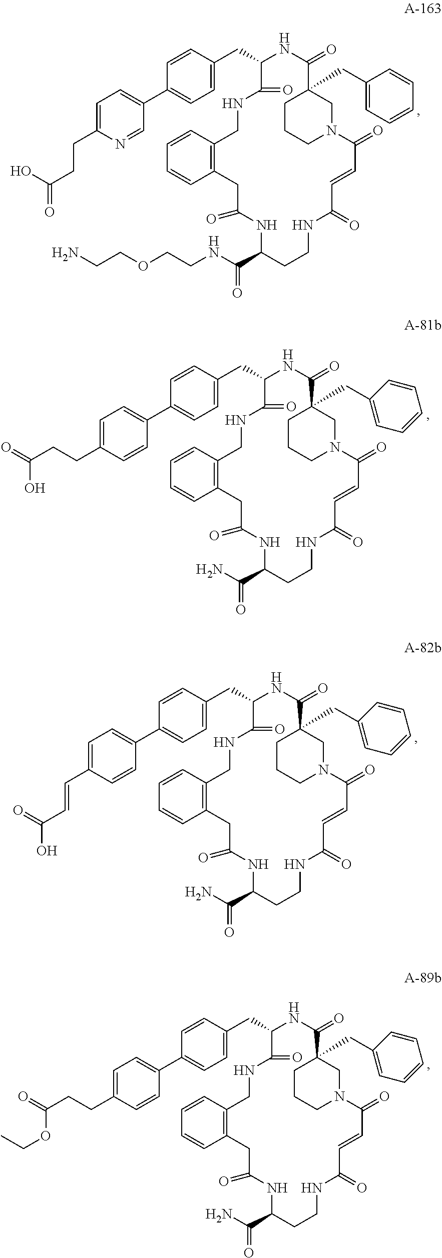

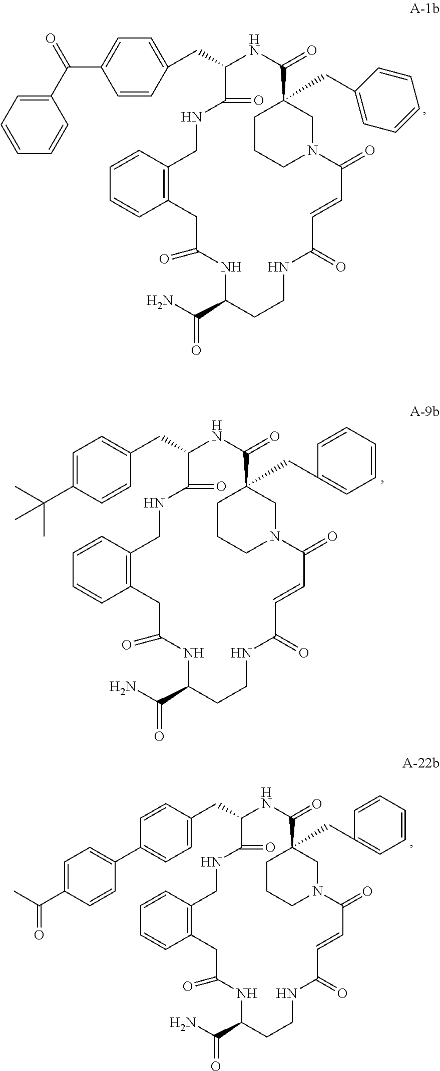 [SMILES: NC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1 ; CC(C)(C)c1ccc(C[C@@H]2NC(=O)[C@]3(Cc4ccccc4)CCCN(C3)C(=O)/C=C/C(=O)NCC[C@@H](C(N)=O)NC(=O)Cc3ccccc3CNC2=O)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1]

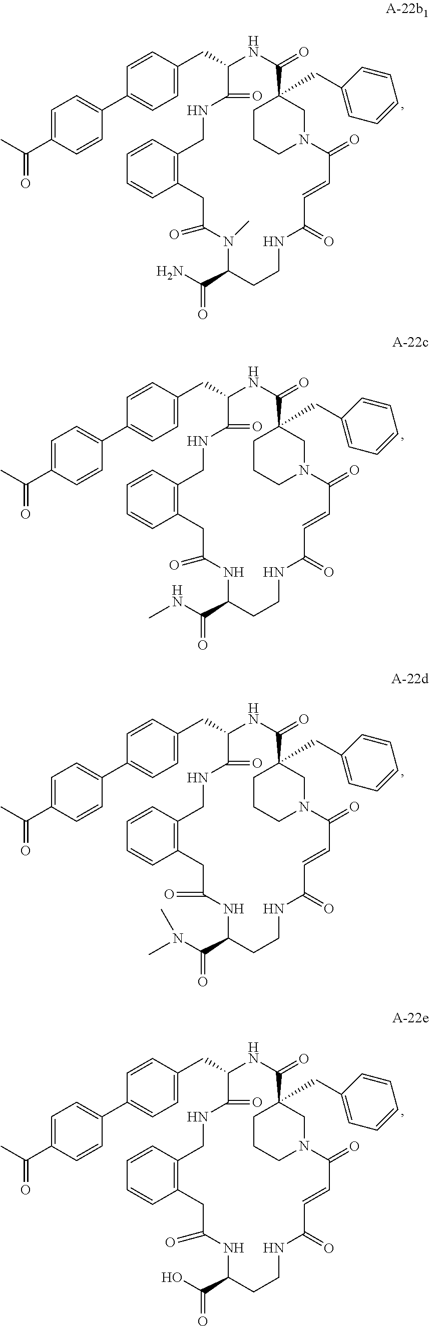 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(N)=O)N(C)C(=O)Cc4ccccc4CNC3=O)cc2)cc1 ; CNC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)N(C)C)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)O)NC(=O)Cc4ccccc4CNC3=O)cc2)cc1]

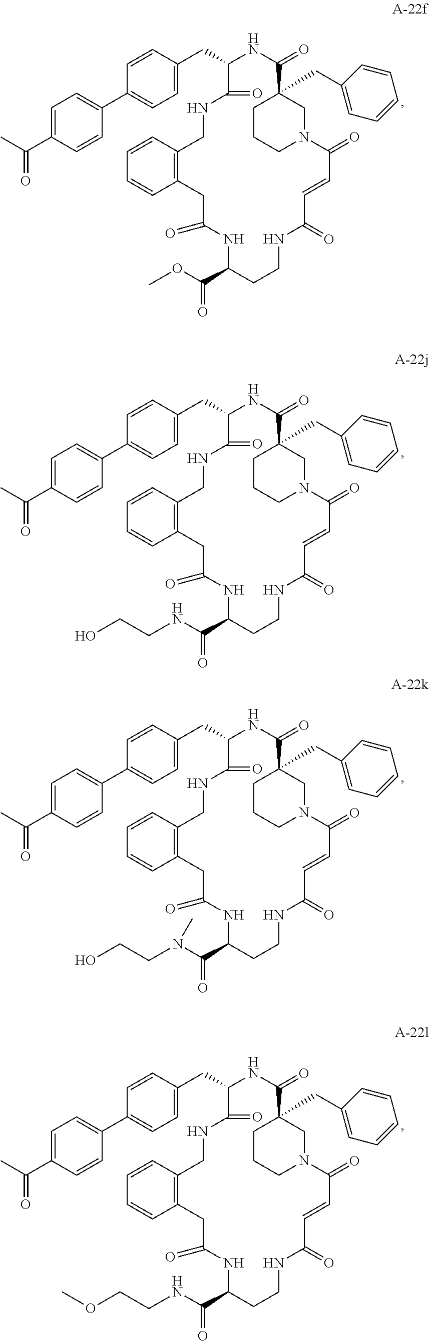 [SMILES: COC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)NCCO)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)N(C)CCO)NC(=O)Cc4ccccc4CNC3=O)cc2)cc1 ; COCCNC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1]

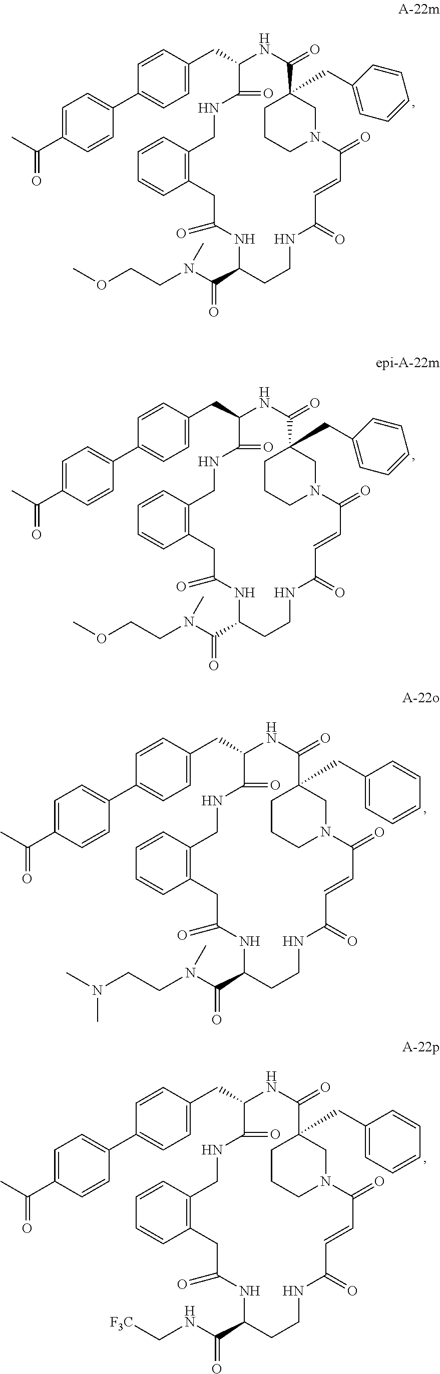

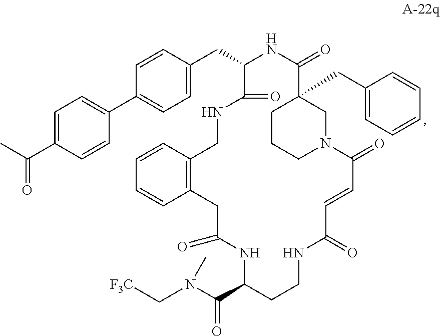 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(=O)N(C)CC(F)(F)F)NC(=O)Cc4ccccc4CNC3=O)cc2)cc1]

and pharmaceutically acceptable salts, solvates, hydrates, polymorphs, co-crystals, tautomers, stereoisomers, isotopically labeled derivatives, and prodrugs thereof.

Exemplary compounds of Formula (I′) or (I) include, but are not limited to, compounds disclosed in Examples 1, 2, and 3. Exemplary compounds of Formula (I′) or (I) include, but are not limited to, compounds disclosed in Examples 1 and 2.

In another aspect, the present disclosure provides pharmaceutical compositions including a compound described herein (e.g., compounds of Formula (I′) or (I) and compounds of Table 1), and optionally a pharmaceutically acceptable excipient. In certain embodiments, a pharmaceutical composition described herein includes a therapeutically or prophylactically effective amount of a compound described herein. The pharmaceutical compositions may be useful in reducing oxidative stress in a subject or cell, inhibiting a cyclophilin (e.g., CypB, CypC, CypD, CypE, CypG, CypH, Cyp40, PPWD1, PPIL1, NKTR) in a subject or cell, in treating and/or preventing a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, a pharmaceutical composition described herein including a compound described herein (e.g., compounds of Table 1) includes a therapeutically or prophylactically effective amount of a compound described herein. The pharmaceutical compositions may be useful in reducing oxidative stress in a subject or cell, inhibiting a cyclophilin (e.g., CypB, CypC, CypD, CypE, CypG, CypH, Cyp40, PPWD1, PPIL1, NKTR) in a subject or cell, in treating and/or preventing a disease (e.g., neurodegenerative disease (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD)) in a subject in need thereof. In certain embodiments, the compound being administered or used inhibits a cyclophilin (e.g., CypB, CypC, CypD, CypE, CypG, CypH, Cyp40, PPWD1, PPIL1, NKTR) in a subject or cell, treats and/or prevents a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, the compound being administered or used (e.g., compounds of Table 1) inhibits a cyclophilin (e.g., CypB, CypC, CypD, CypE, CypG, CypH, Cyp40, PPWD1, PPIL1, NKTR) in a subject or cell, treats and/or prevents a disease (e.g., neurodegenerative disease (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD)) in a subject in need thereof.

In still another aspect, described herein are kits including a container with a compound or pharmaceutical composition described herein. A kit described herein may include a single dose or multiple doses of the compound or pharmaceutical composition. The described kits may be useful in reducing oxidative stress in a subject or cell. The described kits may be useful in inhibiting a cyclophilin (e.g., CypB, CypC, CypD, CypE, CypG, CypH, Cyp40, PPWD1, PPIL1, NKTR) in a subject or cell. The described kits may be useful in modulating (e.g., regulating) the mPTP and/or reducing oxidating stress. The described kits may be useful in inhibiting a cyclophilin (e.g., CypD). The described kits (e.g., including compounds of Formula (I′)) may be useful in treating and/or preventing a disease described herein (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), cardiovascular disease, heart disease, stroke, heart attack, conditions associated with oxidative stress, mitochondrial diseases), or other diseases associated with cyclophilins (e.g., CypD)) in a subject in need thereof. The described kits (e.g., including compounds of Formula (I′) or (I) and compounds of Table 1) may be useful in treating and/or preventing a disease described herein (e.g., neurodegenerative disease (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD)) in a subject in need thereof. In certain embodiments, a kit described herein further includes instructions for using the compound or pharmaceutical composition included in the kit. A kit described herein may also include information (e.g. prescribing information) as required by a regulatory agency, such as the U.S. Food and Drug Administration (FDA).

In certain embodiments, the compound being administered or used inhibits a cyclophilin (e.g., CypB, CypC, CypD, CypE, CypG, CypH, Cyp40, PPWD1, PPIL1, NKTR) in a subject or cell. In certain embodiments, the compound being administered or used inhibits a cyclophilin (e.g., CypD). In certain embodiments, the compound being administered or used inhibits CypD specifically.

Another aspect of the present disclosure relates to methods of treating a disease in a subject in need thereof comprising administering to the subject a therapeutically effective amount of a compound or pharmaceutical composition described herein. In another aspect, the present disclosure provides methods of preventing a disease in a subject in need thereof comprising administering to the subject a prophylactically effective amount of a compound or pharmaceutical composition described herein.

In yet another aspect, the present disclosure provides compounds and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject or cell, a method of reducing oxidative stress in a subject or cell, and a method of treating and/or preventing a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In yet another aspect, the present disclosure provides compounds (e.g., including compounds of Formula (I′) or (I) and compounds of Table 1) and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject or cell, a method of reducing oxidative stress in a subject or cell, and a method of treating and/or preventing a disease (e.g., neurodegenerative disease (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD)) in a subject in need thereof. In another aspect, the present disclosure provides compounds and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject or cell, a method of reducing oxidative stress in a subject or cell, and a method of treating and/or preventing a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In another aspect, the present disclosure provides compounds (e.g., including compounds of Formula (I′) or (I) and compounds of Table 1) and pharmaceutical compositions described herein for use in a method of the disclosure (e.g., a method of inhibiting a cyclophilin (e.g., CypB, CypC, CypD, CypE, CypG, CypH, Cyp40, PPWD1, PPIL1, NKTR) in a subject or cell, a method of reducing oxidative stress in a subject or cell, and a method of treating and/or preventing a disease (e.g., neurodegenerative disease (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD)) in a subject in need thereof.

The present application refers to various issued patents, published patent applications, journal articles, and other publications, all of which are incorporated herein by reference. The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N.Y., 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“Hydrocarbon chain” refers to a substituted or unsubstituted divalent alkyl, alkenyl, or alkynyl group. A hydrocarbon chain includes at least one chain, each node (“carbon unit”) of which including at least one carbon atom, between the two radicals of the hydrocarbon chain. For example, hydrocarbon chain —C^{A}H(C^{B}H₂C^{C}H₃)— includes only one carbon unit C^{A}. The term “Cₓ hydrocarbon chain,” wherein x is a positive integer, refers to a hydrocarbon chain that includes x number of carbon unit(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

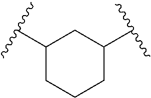 [SMILES: *C1CCCC(*)C1]

is a C₃ hydrocarbon chain. When a range of values is used, e.g., a C₁₋₆ hydrocarbon chain, the meaning of the range is as described herein. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

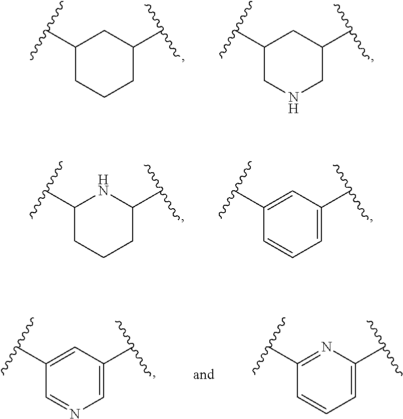 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments

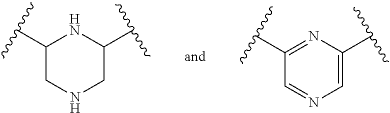 [SMILES: *C1CNCC(*)N1 ; *c1cncc(*)n1]

are not within the scope of the hydrocarbon chains described herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

“Alkenyl” refers to a radical of a straight-chain or branched hydrocarbon group having from 1 to 20 carbon atoms one or more carbon-carbon double bonds (e.g., 1, 2, 3, or 4 double bonds), and no triple bonds (“C₁₋₂₀ alkenyl”). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. In certain embodiments, the alkynyl group is an optionally substituted C₂₋₂₀ alkenyl. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

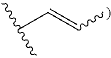

may be in the (E)- or (Z)-configuration.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl. In certain embodiments, the alkynyl group is an optionally substituted C₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (Cm), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined herein. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

A group is optionally substituted unless expressly provided otherwise. The term “optionally substituted” refers to being substituted or unsubstituted.

In certain embodiments, alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}—, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R_{cc})₂, —SO₂R^{aa}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R)₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃-10 carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb)}₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkoxy” or “alkoxyl” refers to a radical of the formula: —O-alkyl.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R—, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I′) or (I), which have cleavable groups and become by solvolysis or under physiological conditions the compounds of Formula (I′) or (I) which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration” refer to implanting, absorbing, ingesting, injecting, inhaling, or otherwise introducing an inventive compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound of Formula (I′) or (I) refers to an amount sufficient to elicit the desired biological response, i.e., treating the condition. As will be appreciated by those of ordinary skill in this art, the effective amount of a compound of Formula (I′) or (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I′) or (I) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for binding a cyclophilin (e.g., CypB, CypC, CypD, CypE, CypG, CypH, Cyp40, PPWD1, PPIL1, NKTR). In certain embodiments, a therapeutically effective amount is an amount sufficient for treating a disease and/or condition (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with a cyclophilin). In certain embodiments, a therapeutically effective amount is an amount sufficient for binding and/or inhibiting a cyclophilin (e.g., CypB, CypC, CypD, CypE, CypG, CypH, Cyp40, PPWD1, PPIL1, NKTR). In certain embodiments, a therapeutically effective amount is an amount sufficient for binding and/or inhibiting a cyclophilin (e.g., CypD).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a cyclophilin (e.g., CypB, CypC, CypD, CypE, CypG, CypH, Cyp40, PPWD1, PPIL1, NKTR) and/or inhibiting the cyclophilin (e.g., CypB, CypC, CypD, CypE, CypG, CypH, Cyp40, PPWD1, PPIL1, NKTR). In certain embodiments, a prophylactically effective amount is an amount sufficient for binding a cyclophilin (e.g., CypD) and/or inhibiting the cyclophilin (e.g., CypD). In certain embodiments, a prophylactically effective amount is an amount sufficient for treating a disease and/or condition (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)).

In certain embodiments, a prophylactically effective amount is an amount sufficient for binding a cyclophilin (e.g., CypB, CypC, CypD, CypE, CypG, CypH, Cyp40, PPWD1, PPIL1, NKTR) and/or inhibiting the cyclophilin (e.g., CypB, CypC, CypD, CypE, CypG, CypH, Cyp40, PPWD1, PPIL1, NKTR), and treating and/or preventing a disease and/or condition (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)). In certain embodiments, a prophylactically effective amount is an amount sufficient for binding a cyclophilin (e.g., CypD) and/or inhibiting the cyclophilin (e.g., CypD), and treating and/or preventing a disease and/or condition (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)).

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). The term “neurodegenerative disease” refers to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, lymphoma, non-Hodgkin's lymphoma, Waldenstrom macroglobulinemia, MYD88-mutated Waldenstrom macroglobulinemia, activated B-cell diffuse large B-cell lymphoma, leukemia. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary original tumor to another organ or tissue and is typically identifiable by the presence of a “secondary tumor” or “secondary cell mass” of the tissue type of the primary original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), MYD88-mutated Waldenstrom macroglobulinemia, activated B-cell (ABC) diffuse large B-cell lymphoma, mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

The “mitochondrial permeability transition pore” (mPTP) is a protein within the inner membrane of the mitochondria that is permeable to molecules less than 1.5 kDa. The mPTP is usually closed, but may be opened under certain conditions including mitochondrial matrix Ca²⁺ accumulation, adenine nucleotide depletion, increased phosphate concentration, or oxidative stress. The opening of the mPTP pore is associated with apoptosis. Cyclophilins (e.g., CypD) can regulate the opening and closing of the mPTP.

“Autophagy” relates to a self-degradation maintenance process in a cell where the cell breaks down and destroys old, damaged, or abnormal proteins and/or other substances in its cytoplasm, to keep the cell functioning properly. Three exemplary types of autophagy include: pexophagy, autophagy selective for degradation of peroxisomes; mitophagy, autophagy selective for degradation of mitochondria; and xenophagy, autophagy selective for degradation of intracellular bacteria and viruses. Exemplary diseases and/or conditions associated with autophagy include, but are not limited to, neurodegenerative disease (e.g., Alzheimer's disease, multiple sclerosis, Parkinson's disease, Huntington's disease), infection (e.g., infection by bacteria, viruses, microbes), cancer, aging, and heart disease.

“Aging” is a phenomenon characterized by progressive accumulation of dysfunctional proteins and damaged organelles at the cellular level. Increased malfunction in the cellular regulatory processes for maintenance, repair, and turnover of defective protein structures is thought to be associated with aging. With age, autophagy activity appears to decline and contributes to the accumulation of cellular components associated with aging.

“Cardiovascular disease” refers any disease or disorder relating to the heart and blood vessels, including, but not limited to hypertension (high blood pressure), coronary heart disease (heart attack), cerebrovascular disease (stroke), peripheral vascular disease, heart failure, rheumatic heart disease, congenital heart disease, and cardiomyopathies. In certain embodiments, cardiovascular disease is caused by “oxidative stress” (e.g., increased production of reactive oxygen species (ROS)). In certain embodiments, a “cardiovascular” condition is an ischemia-reperfusion injury.

“Ischemia-reperfusion injury” refers to the injury characterized by cellular dysfunction and death, after restoration of blood flow to ischemic tissues. “Ischemia” refers to a state where the tissues have a lower than normal blood supply (e.g., resulting in a deficiency of oxygen, glucose, and other materials required for metabolism). “Reperfusion injury” refers to the restoration of blood flow to damaged tissues (e.g., damaged myocardium) which triggers additional ischemic cellular damage.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

A “cyclophilin” is a protein from the cyclophilin family, a group of 17 proteins characterized by a highly conserved peptidyl-prolyl-isomerase domain. A majority of the cyclophilin family members possess enzymatic activity to convert between cis and trans proline-peptide bonds. Cyclophilin D (CypD) acts as a regulator of the mitochondrial permeability transition pore (mPTP), a channel across the inner mitochondrial membrane where prolonged opening results in cell necrosis. For CypD, an exemplary sequence from GenBank is: P30405.1 (Homo sapiens). Other exemplary cyclophilins include, but are not limited to, CypB, CypC, CypD, CypE, CypH, and Cyp40.

### BRIEF DESCRIPTION OF THE DRAWINGS

FIG. 1 is an overview of cyclophilin (e.g., Cyclophilin D) function and activity.

FIGS. 2A-2C illustrate the design of previous Cyclophilin D inhibitors. FIGS. 2A-2C show that earlier inhibitors were reliant on either binding to a highly conserved active pocket (CsA), or dual binding into the active pocket, losing binding in conserved residues at the base of the S2 pocket, resulting in promiscuous cyclophilin inhibition. FIG. 2C shows different cyclophilins and the corresponding residues, where cyclophilin PPIA is the reference, and PPIF corresponds to CypD.

FIG. 3 shows that the adjacent S2 pocket was diversified across the Cyp family. FIG. 3 shows different cyclophilins and the corresponding residues, where cyclophilin PPIA is the reference, and PPIF corresponds to CypD.

FIG. 4 shows results of the selectivity for Cyclophilin D (CypD-5) using enrichment of the compounds of a 256,000 member DNA-templated macrocycle (DTS) library.

FIGS. 5A-5E show CypD inhibition (IC₅₀ inhibitory data) by enriched hits from DTS library selection. ‘Cis’ and ‘trans’ refer to stereochemistry at the alkene.

FIG. 6 show dose response curves with IC₅₀ inhibitory data by exemplary depicted compounds JOMBtrans and JOBBtransA from the inhibitory library. CypD and 10 other Cyp family members were screened against JOMBtrans. The binding curve was generated from Surface Plasmon Resonance experiments. CypD was covalently immobilized on the chip, and the macrocyclic compound was passed over the surface.

FIGS. 7A-7E show exemplary JOMBtrans derived compound structures and their IC₅₀ inhibitory dose response data. FIGS. 7A-7E shows the structures of exemplary JOMBtrans derived compounds, and IC₅₀ data for CypD inhibition for the depicted JOMBtrans derivatives.

FIGS. 8A-8D show relevant dose response curves of IC₅₀ inhibition and administered compound concentration for good JOMBtrans derivatives (compounds 27, 48, 49, 63A, 64A, 65A, 66A, 68A, 70A, 71A, 74A, 75A, 76A, 77A, 78A, 79A) shown in FIGS. 8A-8D) against 11 cyclophilins (CypB, CypC, CypD, CypE, CypG, CypH, Cyp40, PPWD1, PPIL1, NKTR).

FIGS. 9A-9C show co-crystal structures of exemplary inhibitors. CypD-compound JOMBtrans was solved at 1.8 Å and CypD-compound 49 was solved at 1.02 Å. FIG. 9B shows the binding interactions of JOMBtrans: H-bonds with the backbone of Gly72 and sidechains of Gln63 and Arg55; cation-pi interaction with Arg55 (phenyl); and hydrophobic interactions in both S2 pocket (furan) and active site (phenyl). FIG. 9C shows the binding mode of compound 49, which has the same binding interactions as JOMBtrans, plus H-bonds with the backbone of Asn102 and sidechain of Trp121. It is hypothesized that the altered conformation from benzyl group afforded these new H-bond interactions and a 75-fold increase in potency.

FIGS. 10A-10B show the IC₅₀ inhibitory data for of the specificity analysis of the first lead inhibitor compounds. The benzyl group afforded better potency due to the conformational change in the macrocycle. This did not appear to affect the specificity profile, as compound 49 had approximately 10-fold better specificity for CypD compared to any other cyclophilins. Replacement of the benzyl group with other moieties resulted in the same trend (see dose response curves for exemplary compounds 48, 63A, 64A, 64A, 66A, 68A, 70A, 71A, 74A, 75A, 76A, 77A, 78A, and 79A). The furan on JOMBtrans/49 sits inside the S2 pocket.

FIGS. 11A-11E show the IC₅₀ inhibitory data for the first A-Series macrocycles against the depicted cyclophilins.

FIGS. 12A-12B show the trends in the IC₅₀ inhibitory data compared to concentration of administered compounds for the first depicted A-Series macrocycles (see FIGS. 12A-12B for administered compounds) against the depicted cyclophilins. The results show large moieties offered greater overall specificity. In particular, NKTR, CypG, PPWD1, and CypH all show reduced IC₅₀ compared to CypD. These 4 cyclophilins have more sterically occluded S2 pockets.

FIGS. 13A-13C show the co-crystal structures of compounds A-1, A-5, and A-6. All show the same binding mode as Compound 49. As such, compounds A-1 and A-5 reach deeper into the S2 pocket, and the gatekeeper residues are shown to flip out of the pocket upon binding. Crystal structures confirm that increased specificity for CypD is dependent on deeper binding in S2 pocket. Using compound A-5, biphenyl moieties were diversified to gain interactions with gatekeeper residues of CypD (Ser81, Arg82). The crystal structures of CypD-A-1, CypD-A-5, and CypD-A-6 were solved at 1.2 Å.

FIGS. 14A-14W show a collection of further A-Series compound structures and dose response curves (IC₅₀ inhibitory data compared to concentration of administered compounds) against the depicted cyclophilins.

FIGS. 15A-15D show the dose response curves (IC₅₀ inhibitory data compared to concentration of administered compounds) from the A-Series derivative compounds against the depicted cyclophilins. Results show that compounds with ortho-alkyl biphenyl may offer approximately ten-fold CypE specificity.

FIG. 16 shows that the co-crystal structure of A-57 with CypD A-57 has ortho-methyl dug into S2 pocket, and flips Arg even further out of the pocket. The crystal structure was solved at 1.2 Å. Compounds with carboxylate gave specificity over almost all cyclophilins except CypE. Compounds with ortho-methyl gave approximately ten-fold specificity over CypE by pushing biphenyl closer to non-conserved CypE-Serine.

FIGS. 17A-17I show dose response curves (IC₅₀ inhibitory data compared to concentration of the listed administered compounds) for A-Series derivative compounds against the depicted cyclophilins. Results show compounds with para carbonyl biphenyl offers varying selectivity over CypA, CypC, CypB, Cyp40, PPIL1, and PPWD1. In addition, increased potency for CypD was observed for multiple analogs compared to parent compound (A-5, 0.20 μM). FIG. 17D shows compounds with para carboxy biphenyl offered both very good potency for CypD and very good selectivity. FIGS. 17H and 17I show the compounds A-54, A-57, A-63, A-81, and A-81 have potency and specificity increases with the further extension of the carboxylate moiety.

FIG. 18A-18C show results of an analysis of the interactions between cyclophilins and the gatekeeper residues. FIG. 18A shows the co-crystal structure of a compound (A-5) and demonstrates that para-substituents can be placed near/between Ser/Arg gatekeepers. CypD specificity may derive from, but is not limited to, interactions between the carboxylate derivatives of compound A-5 and the gatekeepers. FIG. 18B shows examples of gatekeeper residue-cyclophilin interactions for the compound A-81. FIG. 18C shows results of the results of IC₅₀ inhibitory data against the depicted cyclophilins, for the A-81 macrocycle compound.

FIGS. 19A-19U show a collection of exemplary A-Series compound structures and their corresponding dose response curves against the depicted cyclophilins, with IC₅₀ inhibitory data compared to the concentration of the administered compounds. FIGS. 19A, 19K, 19P, and 19S show exemplary A-Series compound structures. FIGS. 19B-19J, 19L, 19M-190, 19Q, 19R, 19T, and 19U show dose response curves against the depicted cyclophilins.

FIGS. 20A-20I show the dose response curves (IC₅₀ inhibitory data compared to concentration of the listed administered compounds) from the exemplary A-Series derivative compounds against the depicted cyclophilins.

FIG. 21 shows the co-crystal structure of A-81 with CypD A-81, which maintains the same interactions with CypD as shown in all other previous co-crystal structures in the Figures except JOMBtrans (including the co-crystal structures for compound 49 (FIGS. 9C and 10A), compound A-1 (FIG. 13A), compound A-5 (FIG. 13B), compound A-6 (FIG. 13C), and compound A-57 (FIG. 16) with regard to the active site and S2 pocket. Meanwhile, the compound A-81 also forms H-bonds with the non-gatekeeper Ser119, and a salt bridge with the non-gatekeeper Lys118.

FIGS. 22A-22B show unique exemplary structural features of A-81 binding. For example, the loop that contains the gatekeepers may flip out depending on the ligand present and the side chain of the gatekeepers may drastically change position based on the ligand. FIG. 22A shows small S2 pocket ligands, and FIG. 22B shows large S2 pocket ligands.

FIGS. 23A-23C show results of an analysis of the interactions between cyclophilins and the gatekeeper residues. FIG. 23A shows the co-crystal structure of a compound (A-81) (in the left panel) and different depicted cyclophilins (e.g., CypD, CypA, CypB) with their corresponding specified residues proximal to the ligand carboxylate (right panel), including gatekeeper residues. FIG. 23B shows exemplary residue-cyclophilin interactions for the compound A-81, and IC₅₀ values for the depicted cyclophilins. FIG. 23C shows the IC₅₀ inhibitory data against the depicted cyclophilins, for the macrocyclic compound A-81.

FIG. 24 shows in vitro inhibition of mPTP (via the maximum number of tolerated Ca²⁺ pulses over time, before full mPTP opening) in isolated mice kidney mitochondria by the depicted compounds of cyclosporin A, A-22b, A-81b, and DMSO control.

FIG. 25A shows different cyclophilins and their corresponding residues, where cyclophilin PPIA is the reference, and PPIF corresponds to CypD. FIG. 25B shows different cyclophilins and the corresponding residues, where cyclophilin PPIA is the reference, and PPIF corresponds to CypD.

FIGS. 26A-26B show the dose response curves (IC₅₀ inhibitory data compared to concentration of the listed administered compounds) from the exemplary A-Series derivative compounds against the depicted cyclophilins. These macrocycle compounds are designed using the anchor and carboxylate hypothesis. FIGS. 26A-26B show that the inclusion of the malonate moiety in the A-series compounds provides specificity for CypD, including the compound with a dicarboxylate/malonate (A-160) which shows greatly improved potency compared to A-81.

FIGS. 27A-27C show a comparison of the dose response curves (IC₅₀ inhibitory data compared to concentration of the listed administered cyclophilin inhibitor compounds) from the exemplary A-Series derivative compounds and cyclosporine A against the depicted cyclophilins. FIG. 27A shows that cyclosporine A shows high potency, low specificity, and binding to the active site of CypD. FIG. 27B shows that compound 49 shows low potency, low specificity, and binding to the active site of CypD. FIG. 27C shows that compound A-81 shows high potency, high specificity, and binding to the active site, S2 pocket and gatekeeper region of CypD. FIG. 27D shows that compound A-160 shows high potency, high specificity, and binding to the active site, S2 pocket, and gatekeeper region of CypD.

FIG. 28A shows a collection of exemplary A-Series cyclophilin inhibitor compound structures. FIGS. 28B-280 show dose response curves against the depicted cyclophilins, with IC₅₀ inhibitory data compared to the concentration of the exemplary administered cyclophilin inhibitor compounds.

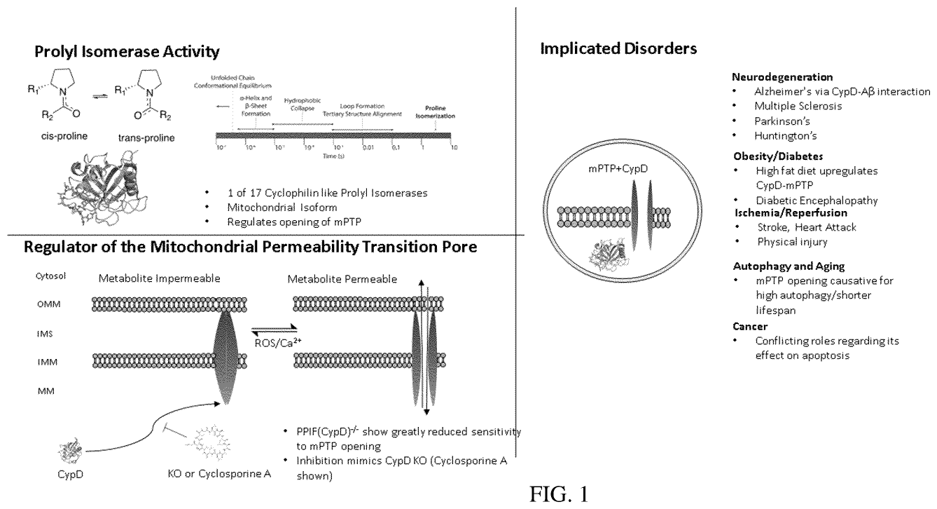

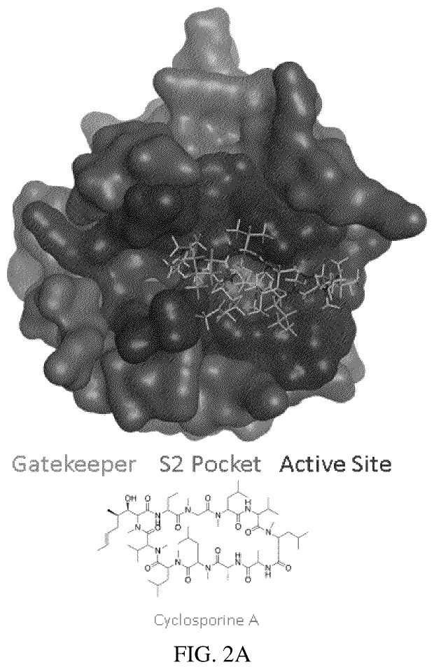

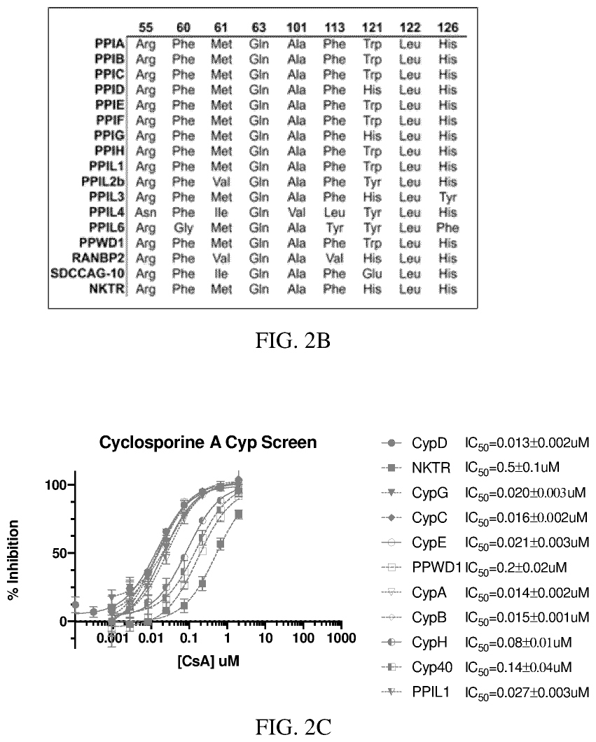

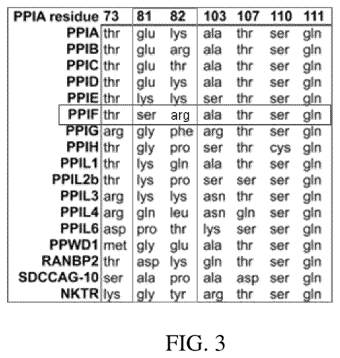

### DETAILED DESCRIPTION OF CERTAIN EMBODIMENTS OF THE INVENTION

The present disclosure provides inhibitors (e.g., selective inhibitors) of cyclophilins (e.g., CypB, CypC, CypD, CypE, CypG, CypH, Cyp40, PPWD1, PPIL1, NKTR). In certain embodiments, the inventive compounds inhibit the activity of CypD. The present disclosure further provides methods of using the compounds described herein, e.g., as biological probes to study the inhibition of the activity of a cyclophilin (e.g., CypB, CypC, CypD, CypE, CypG, CypH, Cyp40, PPWD1, PPIL1, NKTR), and as therapeutics, e.g., in the treatment and/or prevention of diseases associated with the overexpression and/or aberrant activity of the cyclophilin (e.g., CypB, CypC, CypD, CypE, CypG, CypH, Cyp40, PPWD1, PPIL1, NKTR). In certain embodiments, the compounds covalently inhibit a cyclophilin (e.g., CypD). In certain embodiments, the diseases treated and/or prevented include, but are not limited to, neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)). The neurodegenerative diseases include, but are not limited to, Alzheimer's disease, multiple sclerosis, Parkinson's disease, and Huntington's disease. The metabolic disorders include, but are not limited to, obesity and diabetes. The proliferative diseases include, but are not limited to, cancer. Other treated conditions include conditions associated with autophagy and/or aging. The cardiovascular diseases and conditions include, but are not limited to, ischemia-reperfusion injury, stroke, coronary artery disease, and heart attack. In certain embodiments, the condition is a mitochondrial disease, for example, a condition and/or disease associated with the regulation of the mitochondrial permeability transition pore (mPTP) and/or CypD. Also provided by the present disclosure are pharmaceutical compositions, kits, methods, and uses of a compound of Formula (I′) or (I) as described herein.

### Compounds

Certain aspects of the present disclosure relate to the compounds described herein. The compounds described herein may be useful in treating and/or preventing diseases and/or conditions (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)), diseases associated with regulation of the mitochondrial permeability transition pore (mPTP), or diseases associated with the activity of a cyclophilin (e.g., CypD) in a subject, or inhibiting the activity of a cyclophilin (e.g., CypD) in a subject or biological sample. In certain embodiments, a compound described herein is a compound of Formula (I′) or (I), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I′) or (I), or a pharmaceutically acceptable salt thereof.

In certain embodiments, a compound described herein is of Formula (I′):

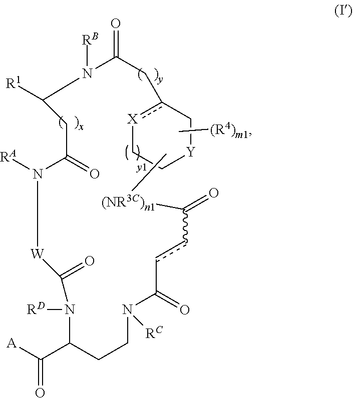

or a pharmaceutically acceptable salt, solvate, hydrate, stereoisomer, polymorph, tautomer, isotopically enriched form, or prodrug thereof, wherein:

each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;

A is —OR^{5A} or —N(R⁵)₂;

W is an optionally substituted C₁₋₆ hydrocarbon chain, optionally wherein one or more carbon units of the hydrocarbon chain are independently replaced with substituted or unsubstituted phenylene, substituted or unsubstituted carbocyclylene, —O—, or —N(R^{W})—;

R¹ is substituted or unsubstituted C₁₋₆ alkyl, substituted or unsubstituted alkenyl, substituted or unsubstituted carbocyclyl, or substituted or unsubstituted aryl;

X is —O—, —C(R^{3A})₂—, —C(R^{3A})═, or —N(R^{3B})—, as valency permits; Y is —C(R^{3A})₂— or —N(R^{3B})—, as valency permits;
each instance of R^{3A} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or optionally wherein two instances of R^{3A} are joined together with the intervening atoms to form an optionally substituted heterocyclyl or heteroaryl ring;

R^{c1} is halogen, hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, —NH₂, —N(optionally substituted alkyl)₂, —OH, or —O(optionally substituted alkyl);

wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

R^{3B} is hydrogen, substituted or unsubstituted acyl, substituted or unsubstituted alkyl, or a nitrogen protecting group;

R^{3C} is hydrogen, substituted or unsubstituted acyl, substituted or unsubstituted alkyl, or a nitrogen protecting group;

R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN;

each instance of R⁵ is independently hydrogen, substituted or unsubstituted acyl, substituted or unsubstituted alkyl, substituted or unsubstituted alkenyl, substituted or unsubstituted alkynyl, or a nitrogen protecting group;

R^{5A} is hydrogen, substituted or unsubstituted acyl, substituted or unsubstituted alkyl, substituted or unsubstituted alkenyl, substituted or unsubstituted alkynyl, or an oxygen protecting group;

each of R^{A}, R^{B}, R^{C}, R^{D}, and R^{W} is independently hydrogen, substituted or unsubstituted alkyl, substituted or unsubstituted acyl, or a nitrogen protecting group;

x is 0 or 1;

y is 0 or 1;

y1 is 0 or 1;

m1 is 0, 1, 2, 3, 4, 5, or 6; and

n1 is 0 or 1;

provided that the compound is not of formula:

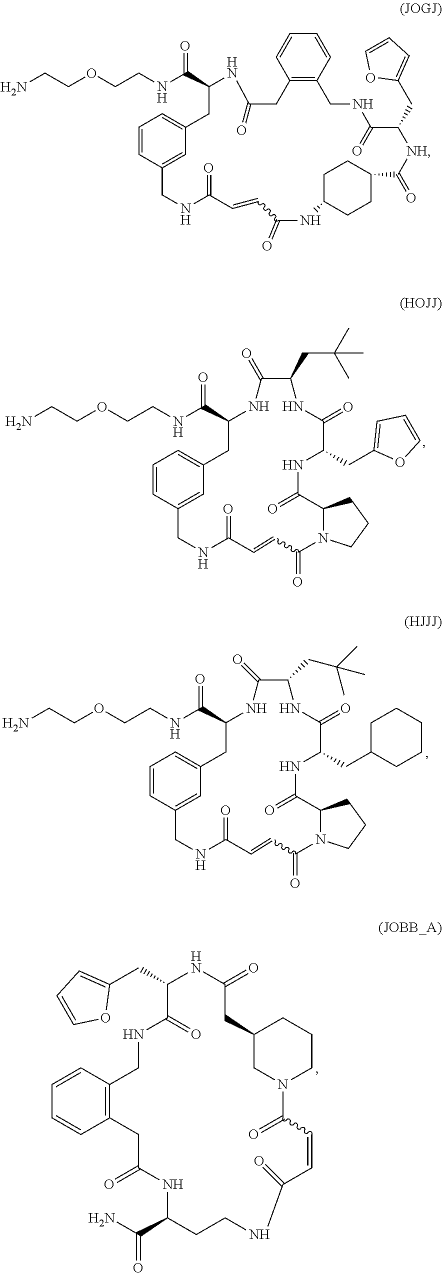 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

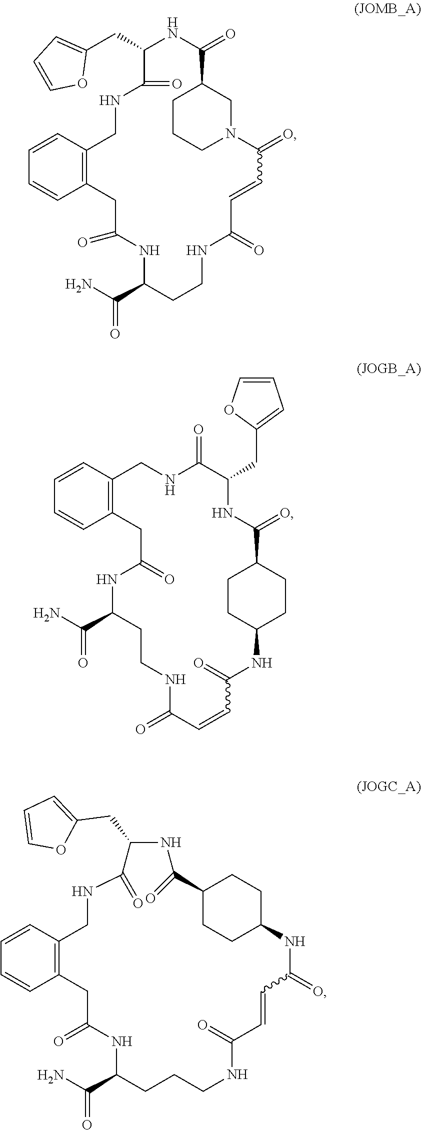 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)NCc2ccccc2CC(=O)N1]

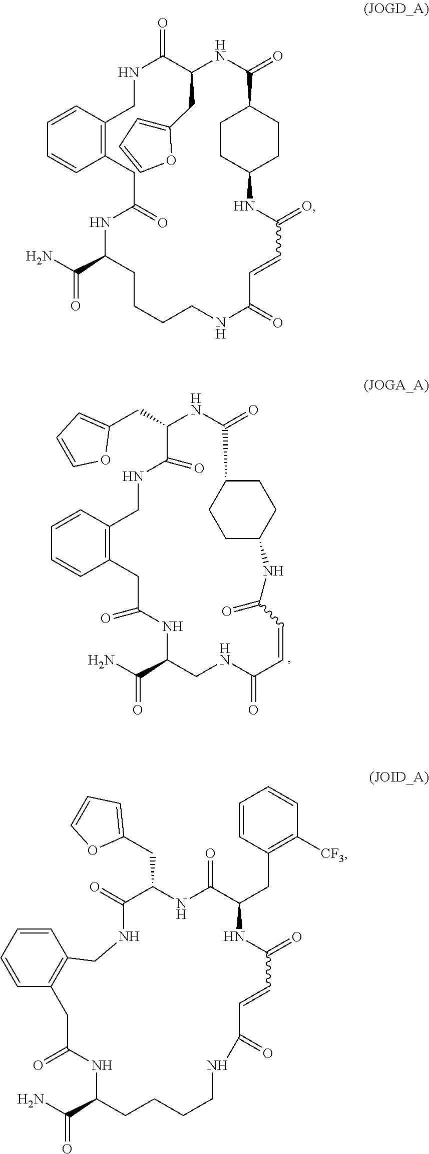 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1]

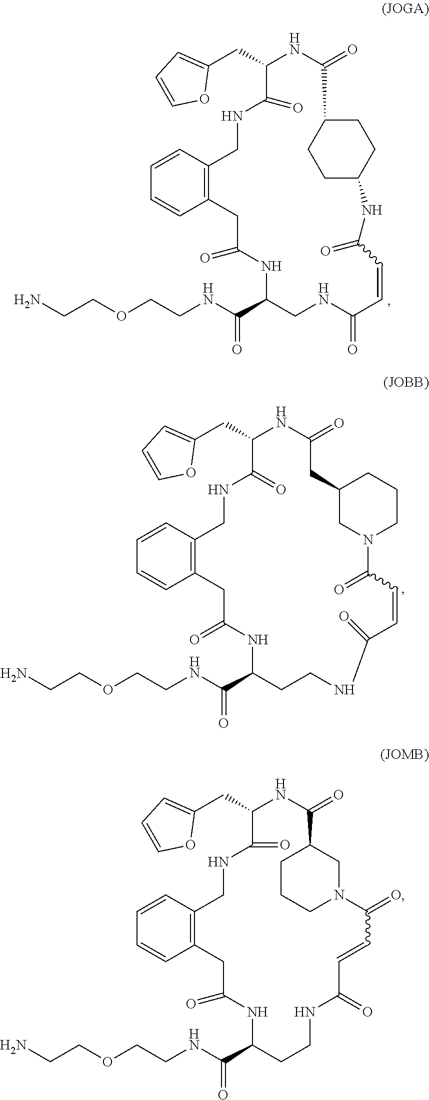 [SMILES: NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NCCOCCNC(=O)[C@@H]1CCNC(=O)C=CC(=O)N2CCC[C@H](CC(=O)N[C@@H](Cc3ccco3)C(=O)NCc3ccccc3CC(=O)N1)C2 ; NCCOCCNC(=O)[C@@H]1CCNC(=O)C=CC(=O)N2CCC[C@H](C2)C(=O)N[C@@H](Cc2ccco2)C(=O)NCc2ccccc2CC(=O)N1]

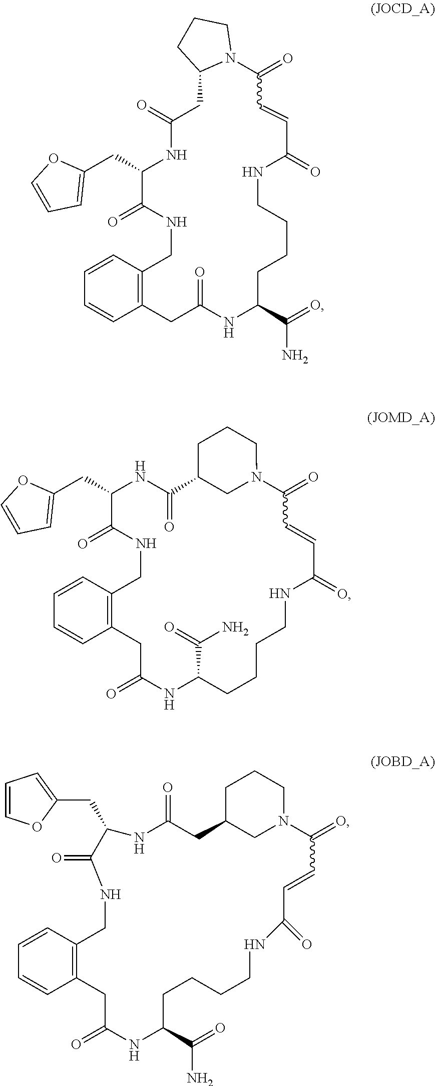 [SMILES: NC(=O)[C@@H]1CCCCNC(=O)C=CC(=O)N2CCC[C@H]2CC(=O)N[C@@H](Cc2ccco2)C(=O)NCc2ccccc2CC(=O)N1 ; NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2]

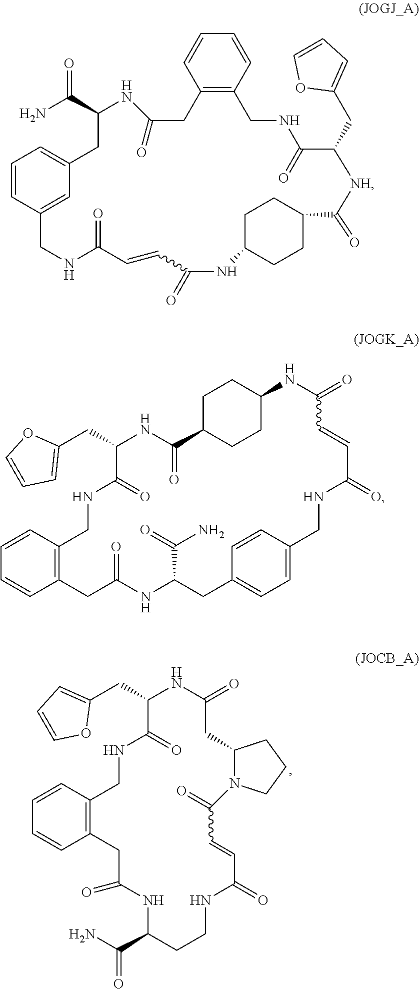 [SMILES: NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1]

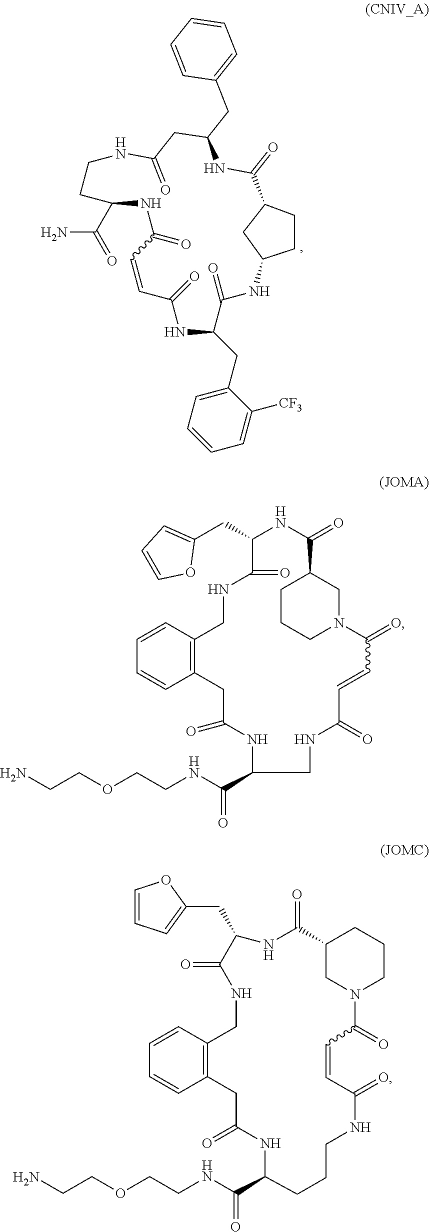 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1]

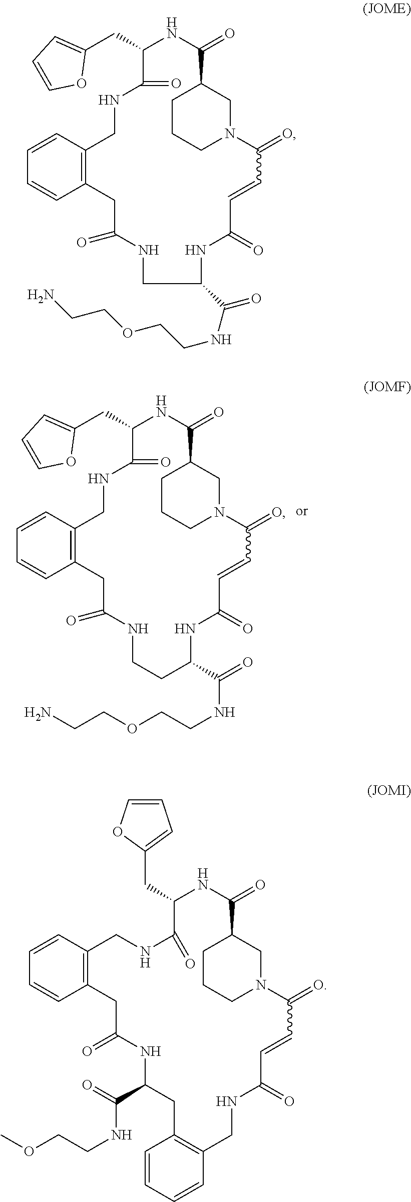 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; COCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

In certain embodiments, a compound described herein is of Formula (I′):

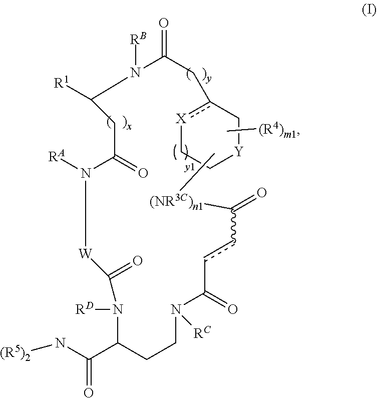

or a pharmaceutically acceptable salt, solvate, hydrate, stereoisomer, polymorph, tautomer, isotopically enriched form, or prodrug thereof, wherein:

each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;

W is optionally substituted C₁₋₆ hydrocarbon chain, optionally wherein one or more carbon units of the hydrocarbon chain are independently replaced with substituted or unsubstituted phenylene, substituted or unsubstituted carbocyclylene, —O—, or —N(R_{W})—;

R¹ is substituted or unsubstituted C₁₋₆ alkyl, substituted or unsubstituted alkenyl, substituted or unsubstituted carbocyclyl, or substituted or unsubstituted aryl;

X is —O—, —C(R^{3A})₂—, —C(R^{3A})═, or —N(R^{3B})—, as valency permits;

Y is —C(R^{3A})₂— or —N(R^{3B})—, as valency permits;

each instance of R^{3A} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or optionally wherein two instances of R^{3A} are joined together with the intervening atoms to form an optionally substituted heterocyclyl or heteroaryl ring;

R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;

wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

R^{3B} is hydrogen, substituted or unsubstituted acyl, substituted or unsubstituted alkyl, or a nitrogen protecting group;

R^{3C} is hydrogen, substituted or unsubstituted acyl, substituted or unsubstituted alkyl, or a nitrogen protecting group;

R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN;

each instance of R⁵ is independently hydrogen, substituted or unsubstituted acyl, substituted or unsubstituted alkyl, substituted or unsubstituted alkenyl, substituted or unsubstituted alkynyl, or a nitrogen protecting group;

each of R^{A}, R^{B}, R^{C}, R^{D}, and R^{W} is independently hydrogen, substituted or unsubstituted alkyl, substituted or unsubstituted acyl, or a nitrogen protecting group;

x is 0 or 1;

y is 0 or 1;

y1 is 0 or 1;

m1 is 0, 1, 2, 3, 4, 5, or 6; and

n1 is 0 or 1;

provided that the compound is not of formula:

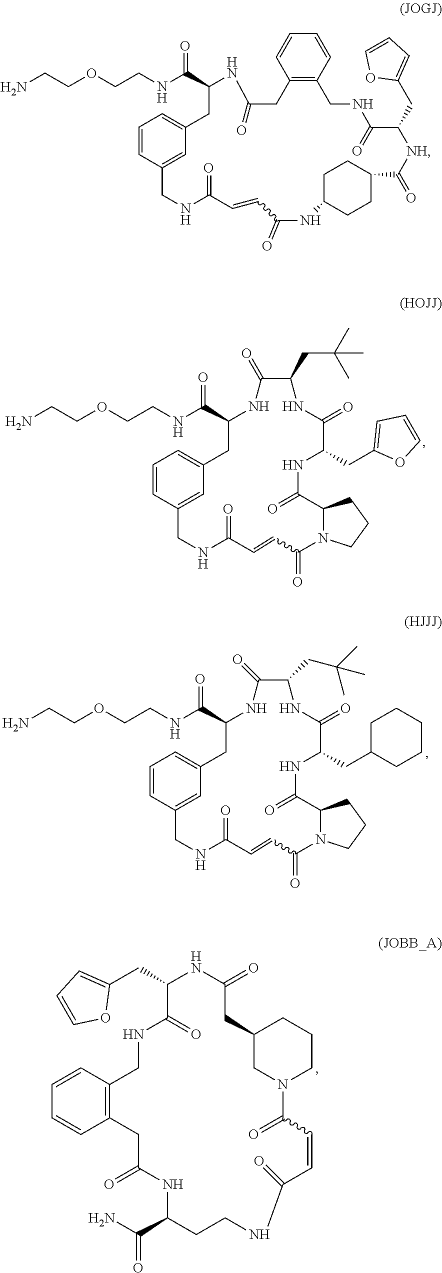 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

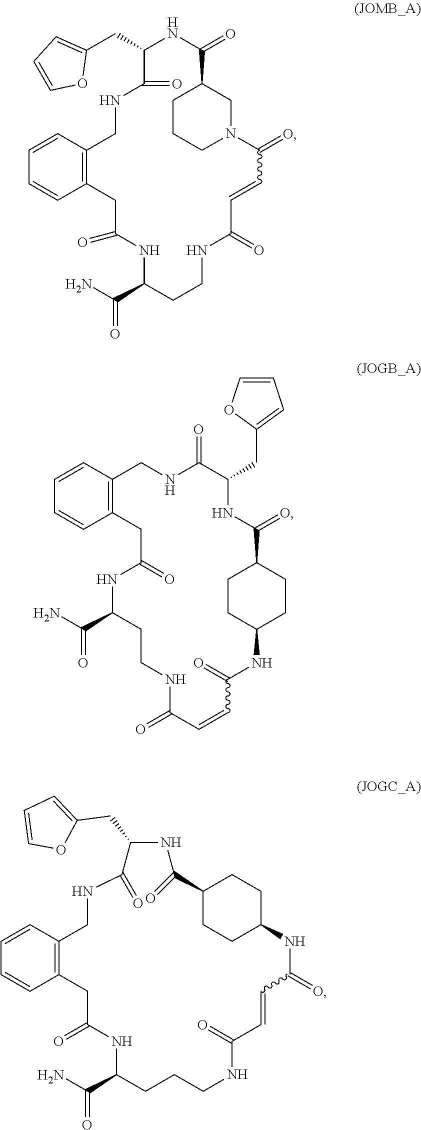 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)NCc2ccccc2CC(=O)N1]

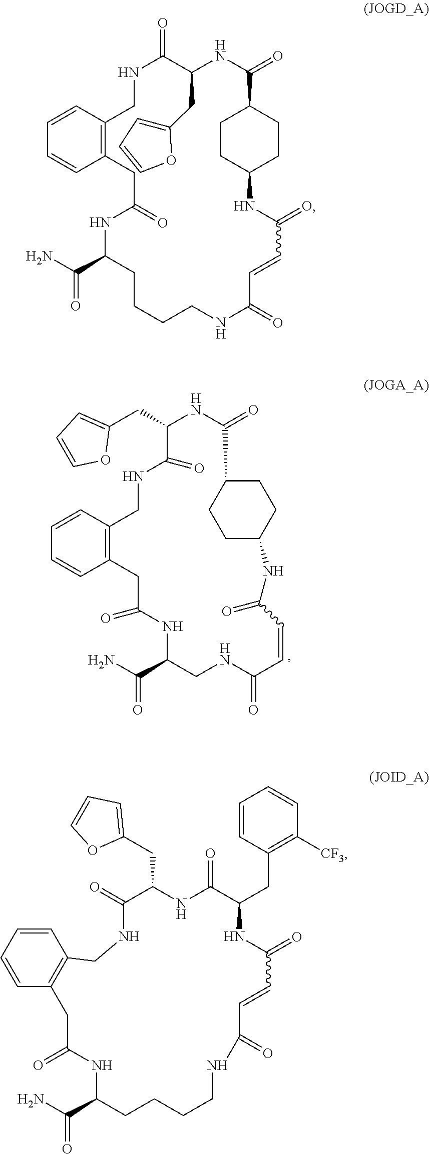 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1]

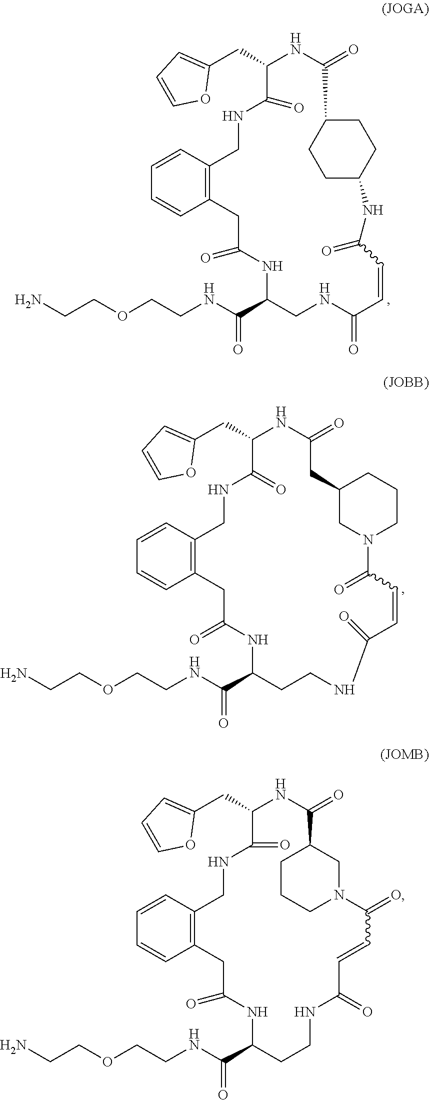 [SMILES: NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NCCOCCNC(=O)[C@@H]1CCNC(=O)C=CC(=O)N2CCC[C@H](CC(=O)N[C@@H](Cc3ccco3)C(=O)NCc3ccccc3CC(=O)N1)C2 ; NCCOCCNC(=O)[C@@H]1CCNC(=O)C=CC(=O)N2CCC[C@H](C2)C(=O)N[C@@H](Cc2ccco2)C(=O)NCc2ccccc2CC(=O)N1]

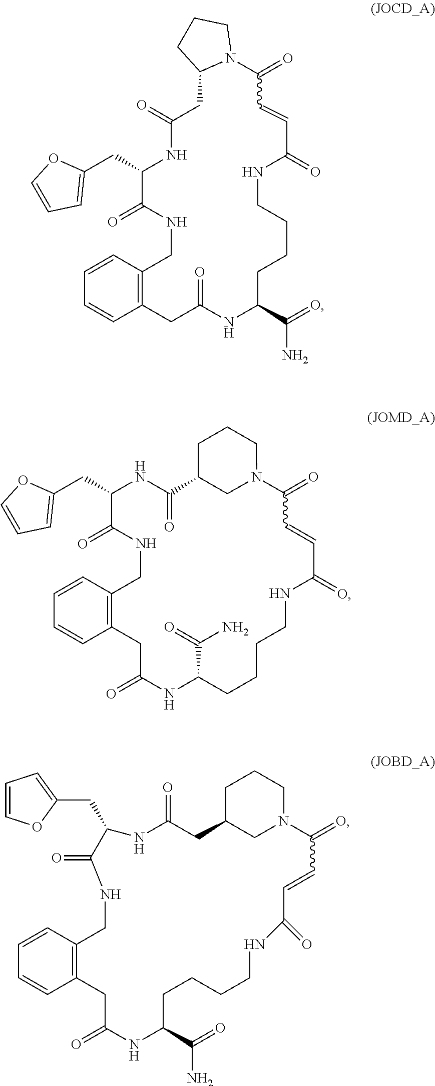 [SMILES: NC(=O)[C@@H]1CCCCNC(=O)C=CC(=O)N2CCC[C@H]2CC(=O)N[C@@H](Cc2ccco2)C(=O)NCc2ccccc2CC(=O)N1 ; NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2]

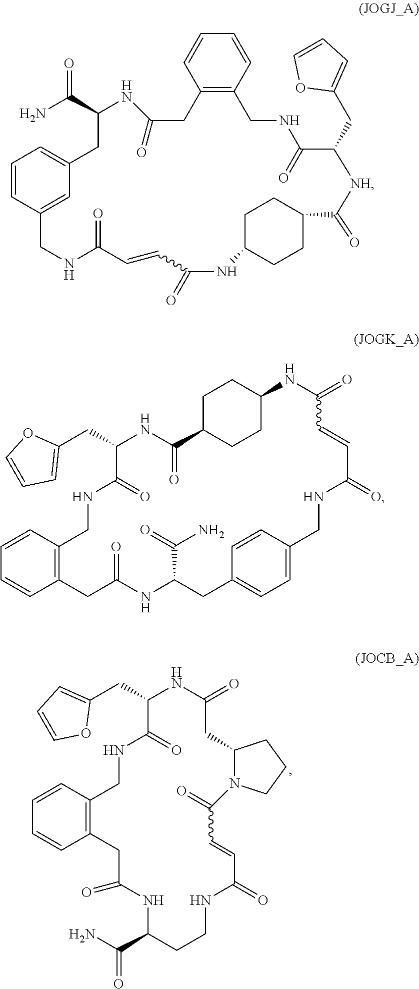 [SMILES: NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1]

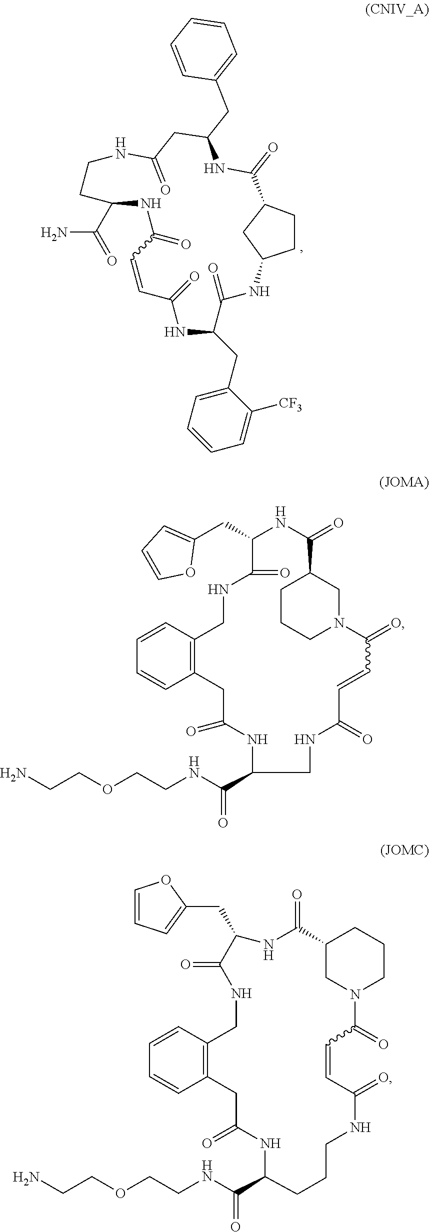 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1]

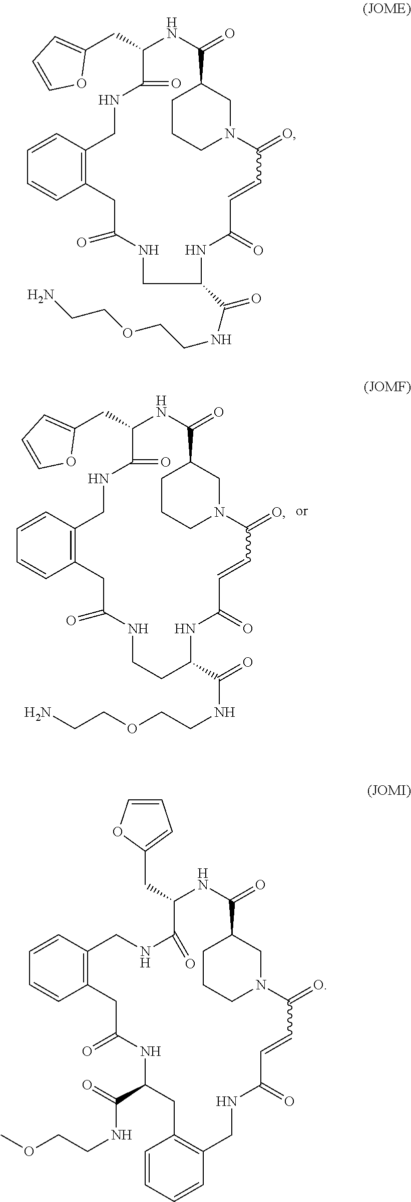 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; COCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

In certain embodiments, the compound of Formula (I′) or (I) is not of formula:

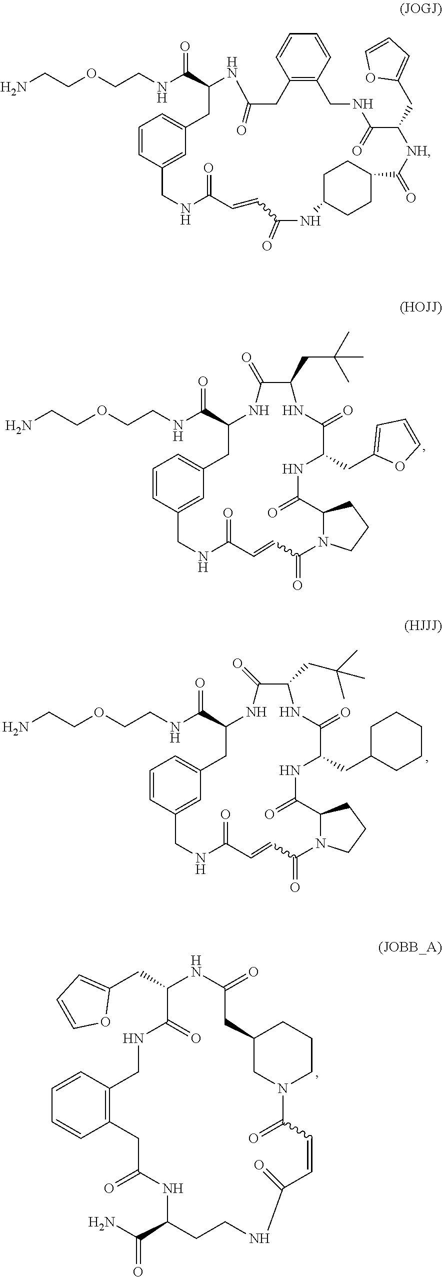 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

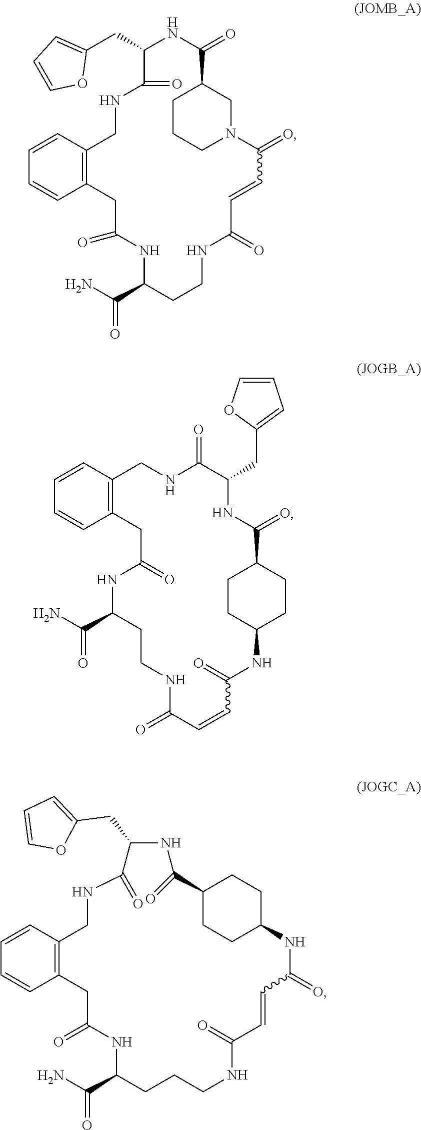 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)NCc2ccccc2CC(=O)N1]

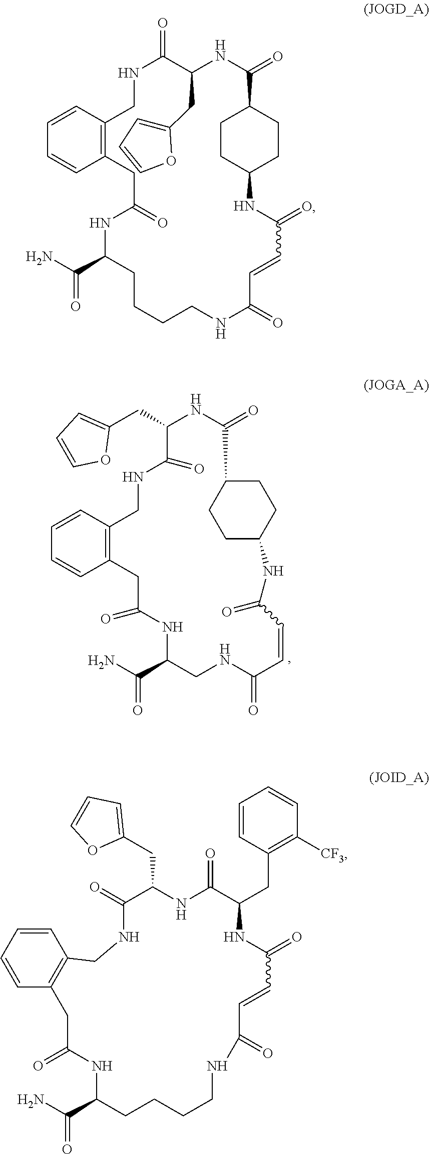 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1]

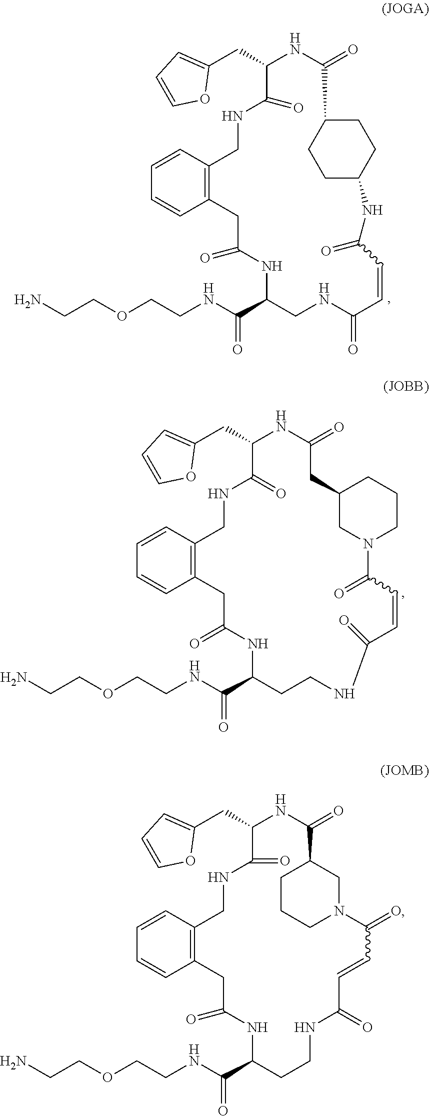 [SMILES: NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NCCOCCNC(=O)[C@@H]1CCNC(=O)C=CC(=O)N2CCC[C@H](CC(=O)N[C@@H](Cc3ccco3)C(=O)NCc3ccccc3CC(=O)N1)C2 ; NCCOCCNC(=O)[C@@H]1CCNC(=O)C=CC(=O)N2CCC[C@H](C2)C(=O)N[C@@H](Cc2ccco2)C(=O)NCc2ccccc2CC(=O)N1]

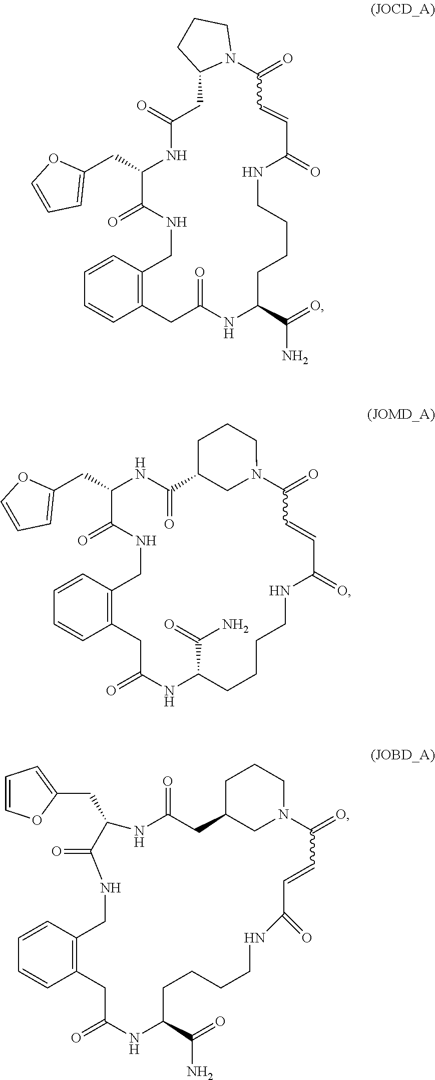 [SMILES: NC(=O)[C@@H]1CCCCNC(=O)C=CC(=O)N2CCC[C@H]2CC(=O)N[C@@H](Cc2ccco2)C(=O)NCc2ccccc2CC(=O)N1 ; NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2]

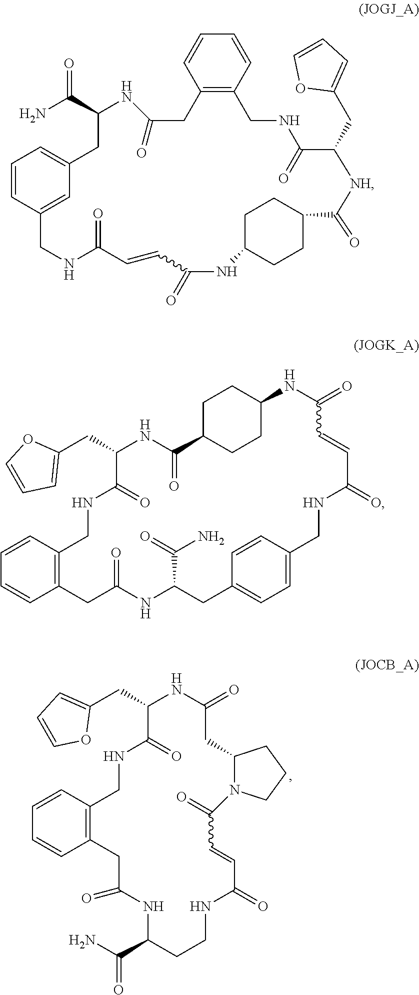 [SMILES: NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1]

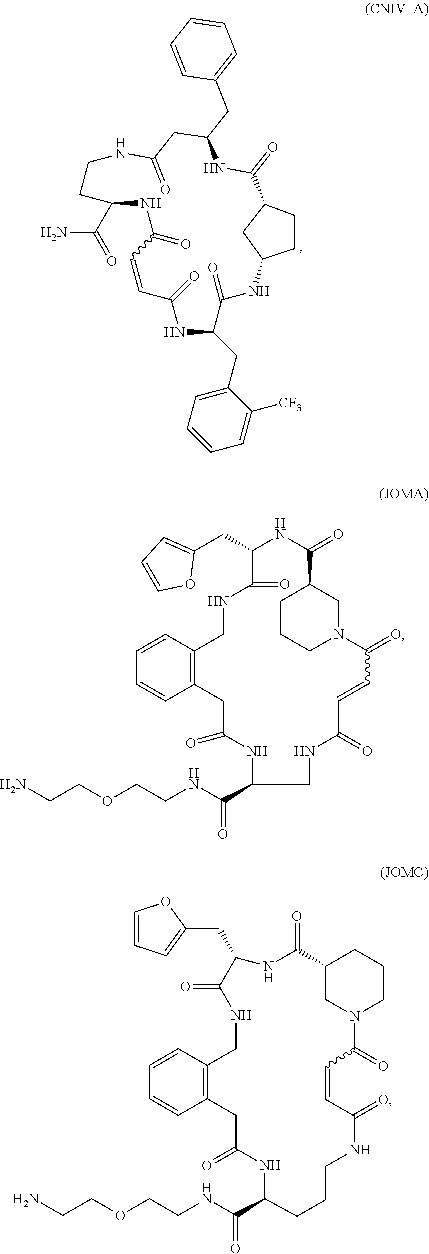 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1]

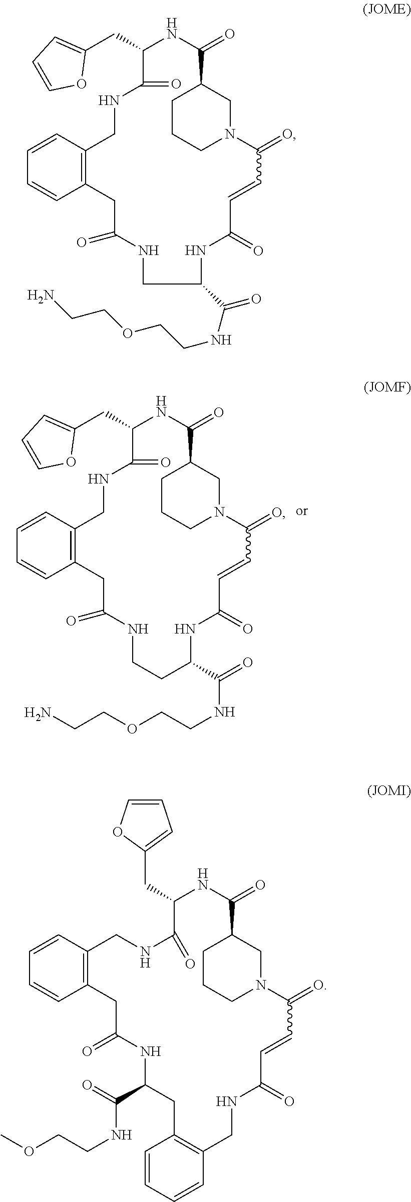 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; COCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

In certain embodiments, a compound described herein is a compound of Formula (I′) or (I), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I′) or (I), or a pharmaceutically acceptable salt thereof.

Compounds of Formula (I′) or (I) include linker W. In certain embodiments, W is an optionally substituted C₁₋₆ hydrocarbon chain, optionally wherein one or more carbon units of the hydrocarbon chain are independently replaced with substituted or unsubstituted phenylene, or substituted or unsubstituted carbocyclylene, —O—, or —N(R^{W})—; and R^{W} is hydrogen, substituted or unsubstituted acyl, substituted or unsubstituted alkyl, or a nitrogen protecting group. In certain embodiments, W is an optionally substituted C₁₋₆ hydrocarbon chain, optionally wherein one or more carbon units of the hydrocarbon chain are independently replaced with substituted or unsubstituted phenylene, or substituted or unsubstituted carbocyclylene, —O—, or —N(R^{W})— (e.g., —NH—). In certain embodiments, W is an optionally substituted C₁₋₆ hydrocarbon chain, optionally wherein one or more carbon units of the hydrocarbon chain are independently replaced with substituted or unsubstituted phenylene, —O—, or —N(R^{W})— (e.g., —NH—).

In certain embodiments, in linker W that is an optionally substituted C₁₋₆ hydrocarbon chain, one or more carbon units of the hydrocarbon chain are independently optionally replaced with —N(R^{W})—, and R^{W} is as defined herein. In certain embodiments, R^{W} is hydrogen. In certain embodiments, R^{W} is substituted or unsubstituted acyl (e.g., —C(═O)Me). In certain embodiments, R^{W} is substituted or unsubstituted alkyl (e.g., substituted or unsubstituted C₁₋₆ alkyl). In certain embodiments, R^{W} is substituted or unsubstituted C₁₋₆ alkyl. In certain embodiments, R^{W} is substituted or unsubstituted methyl. In certain embodiments, R^{W} is unsubstituted methyl. In certain embodiments, R^{W} is substituted or unsubstituted ethyl. In certain embodiments, R^{W} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, W is an optionally substituted C₁₋₆ hydrocarbon chain, optionally wherein one or more carbon units of the hydrocarbon chain are independently replaced with substituted or unsubstituted phenylene or substituted or unsubstituted carbocyclylene (e.g., substituted or unsubstituted, 3- to 10-membered, monocyclic carbocyclyl comprising zero, one, or two double bonds in the carbocyclic ring system)). In certain embodiments, W is substituted or unsubstituted C₁₋₆ alkylene. In certain embodiments, W is substituted or unsubstituted C₁₋₄ alkylene. In certain embodiments, W is substituted or unsubstituted n-butylene, n-pentylene, or n-hexylene. In certain embodiments, W is substituted or unsubstituted n-butylene. In certain embodiments, W is substituted or unsubstituted n-pentylene. In certain embodiments, W is substituted or unsubstituted n-hexylene. In certain embodiments, W is substituted or unsubstituted carbocyclylene (e.g., substituted or unsubstituted, 3- to 10-membered, monocyclic carbocyclyl comprising zero, one, or two double bonds in the carbocyclic ring system)). In certain embodiments, W is substituted or unsubstituted, 5- to 10-membered, monocyclic carbocyclyl comprising zero, one, or two double bonds in the carbocyclic ring system. In certain embodiments, W is of formula:

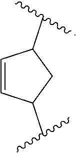 [SMILES: *C1C=CC(*)C1]

In certain embodiments, W is optionally substituted C₁₋₆ hydrocarbon chain, optionally wherein one or more carbon units of the hydrocarbon chain are independently replaced with substituted or unsubstituted phenylene. In certain embodiments, W is of formula:

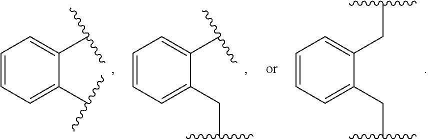

In certain embodiments, W is of formula:

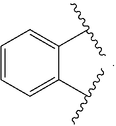 [SMILES: *c1ccccc1* ; "."]

In certain embodiments, W is of formula:

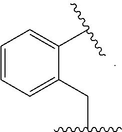 [SMILES: *Cc1ccccc1* ; "."]

In certain embodiments, W is of formula:

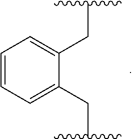 [SMILES: *Cc1ccccc1C* ; "."]

In certain embodiments, W is unsubstituted n-butylene, or of formula:

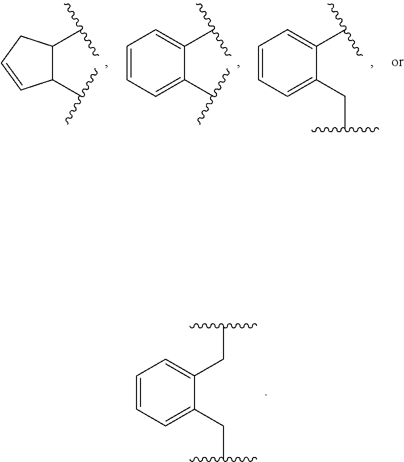

In certain embodiments, x is 0. In certain embodiments, x is 1.

In certain embodiments, R¹ is substituted or unsubstituted C₁₋₆ alkyl (e.g., substituted or unsubstituted methyl, substituted or unsubstituted ethyl, substituted or unsubstituted propyl, substituted or unsubstituted butyl). In certain embodiments, R¹ is C₁₋₆ alkyl optionally substituted with substituted or unsubstituted acyl, substituted or unsubstituted carbocyclyl, substituted or unsubstituted aryl, substituted or unsubstituted heterocyclyl, or substituted or unsubstituted heteroaryl, or —OR^{c1} wherein R^{c1} is as defined herein. In certain embodiments, R¹ is C₁₋₆ alkyl, optionally substituted with substituted or unsubstituted acyl, substituted or unsubstituted alkenyl, substituted or unsubstituted carbocyclyl, substituted or unsubstituted aryl, substituted or unsubstituted heterocyclyl, or substituted or unsubstituted heteroaryl. In certain embodiments, R¹ is methyl. In certain embodiments, R¹ is C₁₋₆ alkyl, optionally substituted with substituted or unsubstituted aryl (e.g., substituted or unsubstituted phenyl). In certain embodiments, R¹ is of formula:

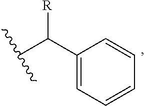

wherein R is independently halogen, substituted or unsubstituted acyl, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted heteroaryl, —OR^{c1}, —NO₂, —N(R^{c2})₂, —N═C(NH₂)₂, —SR^{c1}, —SO₂, —CN, or —SCN. In certain embodiments, R¹ is of formula:

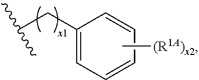

wherein: each instance of R^{1A} is independently halogen, substituted or unsubstituted acyl, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted heteroaryl, —OR^{c1}, —NO₂, —N(R^{c2})₂, —N═C(NH₂)₂, —SR^{c1}, —SO₂, —CN, —SCN, or —OP(═O)(OH)₂, or optionally wherein two instances of R^{1A} are joined together with the intervening atoms to form an optionally substituted aryl or an optionally substituted heteroaryl group; x1 is 0, 1, 2, 3, 4, 5, or 6; and x2 is 0, 1, 2, 3, 4, or 5.

In certain embodiments, R¹ is of formula:

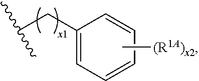

wherein: R^{1A} is halogen, substituted or unsubstituted acyl, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted heteroaryl, —OR^{c1}, —NO₂, —N(R^{c2})₂, —N═C(NH₂)₂, —SR^{c1}, —SO₂, —CN, —SCN, or —OP(═O)(OH)₂, or optionally wherein two instances of R^{1A} are joined together with the intervening atoms to form an optionally substituted aryl group; x1 is 0, 1, 2, 3, 4, 5, or 6; and x2 is 0, 1, 2, 3, 4, or 5. In certain embodiments, R¹ is of formula:

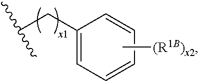

wherein: each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; and x2 is 0, 1, 2, 3, 4, 5, 6, 7, 8, 9, or 10. In certain embodiments, R¹ is of formula:

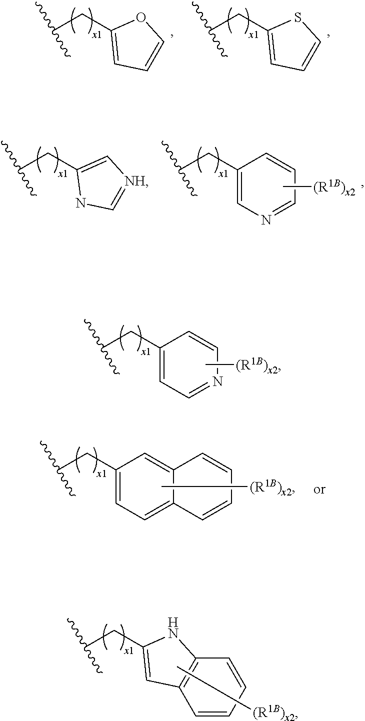

wherein: each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or nitrogen protecting group when attached to a nitrogen atom; x1 is 0, 1, 2, 3, 4, 5, or 6; and x2 is 0, 1, 2, 3, 4, 5, or 6. In certain embodiments, at least one instance of R^{1A} is halogen (e.g., F, Cl, Br, or I). In certain embodiments, at least one instance of R^{1A} is substituted or unsubstituted acyl (e.g.,
—C(═O)Me). In certain embodiments, at least one instance of R^{1A} is substituted or unsubstituted alkyl (e.g., substituted or unsubstituted C₁₋₆ alkyl). In certain embodiments, at least one instance of R^{1A} is substituted or unsubstituted methyl. In certain embodiments, at least one instance of R^{1A} is substituted methyl (e.g., —CF₃). In certain embodiments, at least one instance of R^{1A} is unsubstituted methyl. In certain embodiments, at least one instance of R^{1A} is substituted or unsubstituted ethyl. In certain embodiments, at least one instance of R^{1A} is substituted ethyl (e.g., —CH₂CH₂OH). In certain embodiments, at least one instance of R^{1A} is —CH₂CH₂OH. In certain embodiments, at least one instance of R^{1A} is —CH₂CH₂OMe. In certain embodiments, at least one instance of R^{1A} is unsubstituted ethyl. In certain embodiments, at least one instance of R^{1A} is substituted or unsubstituted propyl. In certain embodiments, at least one instance of R^{1A} is substituted or unsubstituted butyl (e.g., t-butyl, n-butyl). In certain embodiments, at least one instance of R^{1A} is substituted or unsubstituted t-butyl. In certain embodiments, at least one instance of R^{1A} is unsubstituted t-butyl. In certain embodiments, at least one instance of R^{1A} is substituted or unsubstituted alkenyl (e.g., substituted or unsubstituted C₂₋₆ alkenyl). In certain embodiments, at least one instance of R^{1A} is substituted or unsubstituted alkynyl (e.g., substituted or unsubstituted C₂₋₆ alkynyl). In certain embodiments, at least one instance of R^{1A} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{1A} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{1A} is substituted or unsubstituted aryl (e.g., substituted or unsubstituted, 6- to 10-membered aryl). In certain embodiments, at least one instance R^{1A} is benzyl. In certain embodiments, at least one instance of R^{1A} is substituted or unsubstituted phenyl. In certain embodiments, at least one instance of R^{1A} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{1A} is —OR^{c1} (e.g., —OH or —OMe). In certain embodiments, at least one instance of R^{1A} is —O(CH₂)(optionally substituted aryl). In certain embodiments, at least one instance of R^{1A} is —O(CH₂)(phenyl). In certain embodiments, at least one instance of R^{1A} is —O(optionally substituted phenyl). In certain embodiments, at least one instance of R^{1A} is —O(optionally substituted aryl). In certain embodiments, at least one instance of R^{1A} is —NO₂. In certain embodiments, at least one instance of R^{1A} is —N(R^{c2})₂ (e.g., —NMe₂). In certain embodiments, at least one instance of R^{1A} is —N═C(NH₂)₂. In certain embodiments, at least one instance of R^{1A} is —SR^{c1} (e.g., —SMe). In certain embodiments, at least one instance of R^{1A} is —SO₂, —CN, or —SCN. In certain embodiments, at least one instance of R^{1A} is —OP(═O)(OH)₂. In certain embodiments, two instances of R^{1A} are joined together with the intervening atoms to form an optionally substituted aryl group (e.g., substituted or unsubstituted, 6- to 10-membered aryl).

In certain embodiments, at least one instance of x1 is 0. In certain embodiments, at least one instance of x1 is 1. In certain embodiments, at least one instance of x1 is 2. In certain embodiments, at least one instance of x1 is 3. In certain embodiments, at least one instance of x1 is 4. In certain embodiments, at least one instance of x1 is 5. In certain embodiments, at least one instance of x1 is 6. In certain embodiments, at least one instance of x2 is 0. In certain embodiments, at least one instance of x2 is 1. In certain embodiments, at least one instance of x2 is 2. In certain embodiments, at least one instance of x2 is 3. In certain embodiments, at least one instance of x2 is 4. In certain embodiments, at least one instance of x2 is 5.

In certain embodiments, R¹ is of formula:

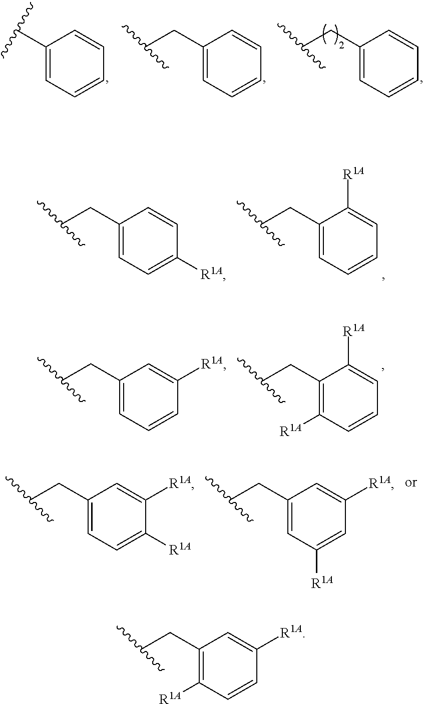

In certain embodiments, R¹ is of formula:

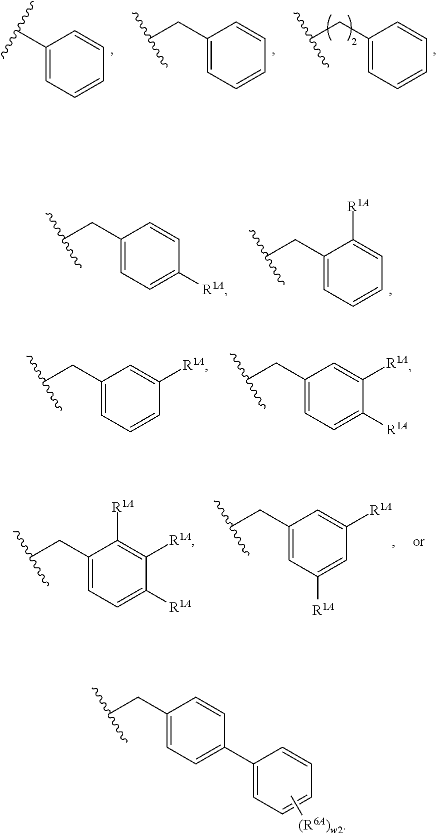

In certain embodiments, R¹ is

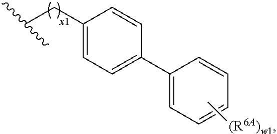

wherein: each instance of R^{6A} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, —B(OR^{c1})₂, or —SCN; x1 is 0, 1, 2, or 3; and w1 is 0, 1, 2, 3, 4, or 5.

In certain embodiments, R¹ is of formula:

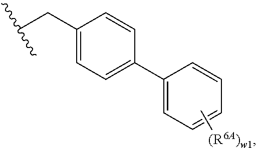

wherein: each instance of R^{6A} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, —B(OH)₂, or —SCN; and w1 is 0, 1, 2, 3, 4, 5, or 6.

In certain embodiments, in the compound of formula:

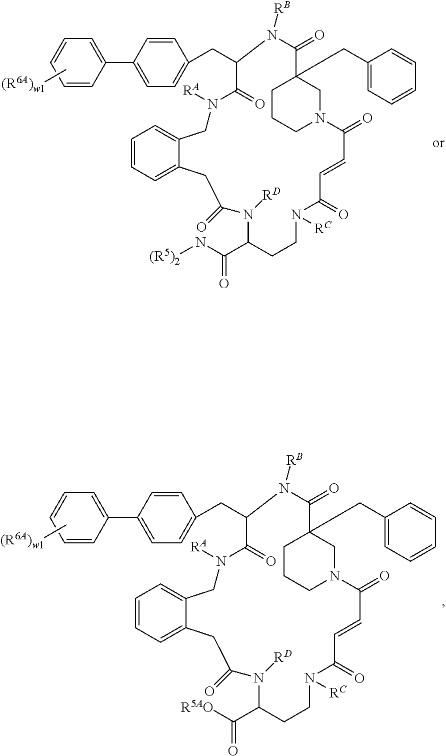

there are zero or more instances of R^{6A}. In certain embodiments, in the compound of formula:

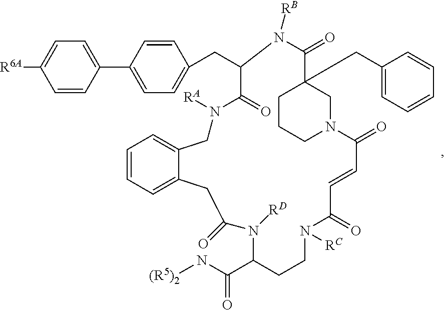

there is one instance of R^{6A}. In certain embodiments, w1 is 0. In certain embodiments, there are one or more instances of R^{6A}. In certain embodiments, w1 is 1. In certain embodiments, at least one instance of w1 is 2. In certain embodiments, at least one instance of w1 is 3. In certain embodiments, at least one instance of w1 is 4. In certain embodiments, at least one instance of w1 is 5. In certain embodiments, at least one instance of w1 is 6. In certain embodiments, at least one instance of R^{6A} is halogen (e.g., F, Cl, Br, or I). In certain embodiments, at least one instance of R^{6A} is substituted or unsubstituted acyl (e.g., —C(═O)Me). In certain embodiments, at least one instance of R^{6A} is substituted or unsubstituted alkyl (e.g., substituted or unsubstituted C₁₋₆ alkyl). In certain embodiments, at least one instance of R^{6A} is methyl. In certain embodiments, at least one instance of R^{6A} is C₁₋₆ alkyl, optionally substituted with substituted or unsubstituted acyl, —OH, —O(C₁₋₆ alkyl), —NH₂, —NH(C₁₋₆ alkyl), —N(C₁₋₆ alkyl)₂, —NO₂, —CN, or —SO₂H. In certain embodiments, at least one instance of R^{6A} is —(CH₂)NH₂, —(CH₂)NHMe, —(CH₂)NMe₂, —(CH₂)₂NH₂, —(CH₂)₂NHMe, —(CH₂)₂NMe₂, or —(CH₂)₂NO₂. In certain embodiments, at least one instance of R^{6A} is:

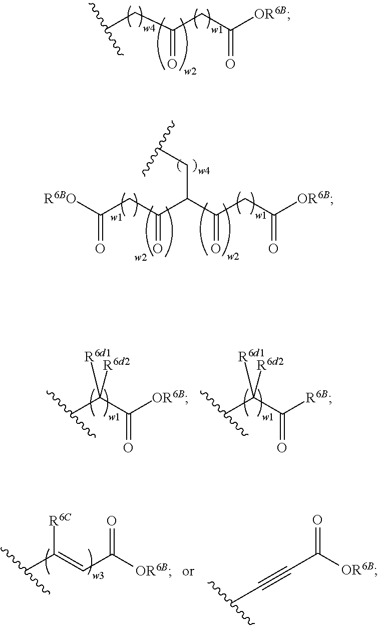

wherein: each instance of w1 is independently 0, 1, 2, 3, 4, 5, or 6; each instance of w2 is independently 1, 2, or 3; w3 is 1, 2, or 3; w4 is 0, 1, 2, or 3; R^{B} is hydrogen or substituted or unsubstituted alkyl; each instance of R⁶ is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN; and

each instance of R^{6d1} and R^{6d2} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN. In certain embodiments, at least one instance of R^{6A} is:

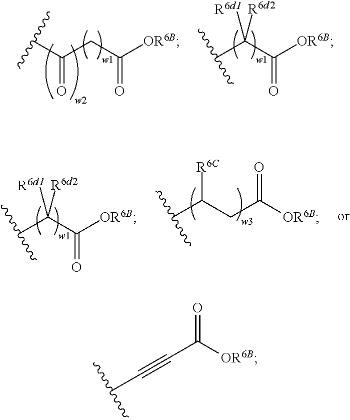

wherein: w1, w2, w3, R^{6B}, R^{6C}, R^{6d1}, and R^{d2} are as defined herein. In certain embodiments, at least one instance of R^{6A} is:

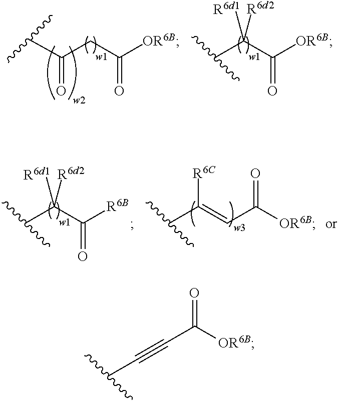

wherein: w1 is 0, 1, 2, 3, 4, 5, or 6; w2 is 1, 2, or 3; w3 is 1, 2, or 3; R^{6B} is hydrogen or substituted or unsubstituted alkyl; each instance of R^{6C} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN; and each instance of R^{6d1} and R^{6d2} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN. In certain embodiments, at least one instance of w1 is 0 or 1. In certain embodiments, at least one instance of w2 is 0 or 1. In certain embodiments, w2 is 1. In certain embodiments, at least one instance of w2 is 2. In certain embodiments, at least one instance of w2 is 3. In certain embodiments, w3 is 1. In certain embodiments, at least one instance of w3 is 2. In certain embodiments, at least one instance of w3 is 3. In certain embodiments, w4 is 1. In certain embodiments, at least one instance of R^{6A} is:

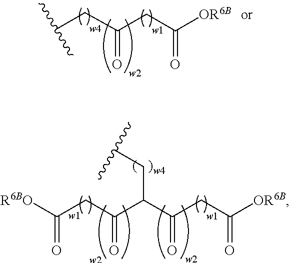

wherein each instance of w1 is independently 0, 1, 2, or 3; each instance of w2 is independently is 1, 2, or 3; w4 is 0, 1, 2, or 3; and R^{6B} is hydrogen or alkyl. In certain embodiments, at least one instance of w1 is 0, and at least one instance of w2 is 0. In certain embodiments, each instance of w1 and w2 is 0, and w4 is 0 or 1. In certain embodiments, R¹ is

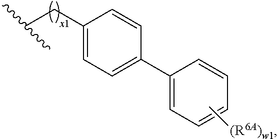

and at least one instance of R^{6A} is:

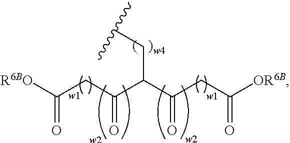

wherein w1, w2, w4, and R^{6B} are as defined herein. In certain embodiments, R¹ is of formula:

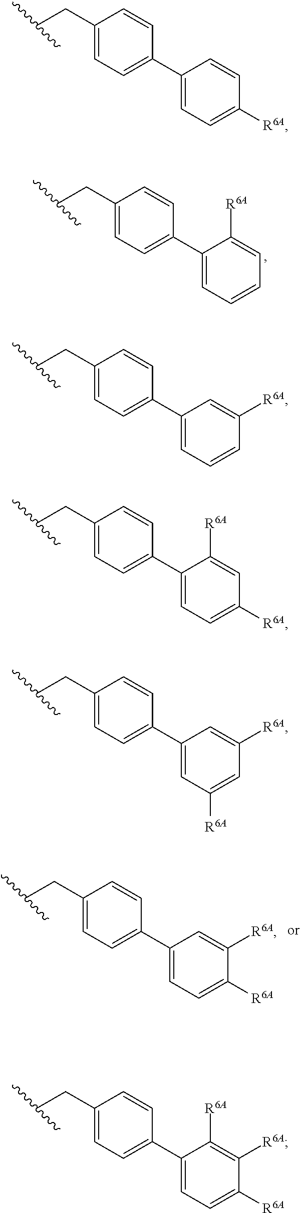

and at least one instance of R^{6A} is:

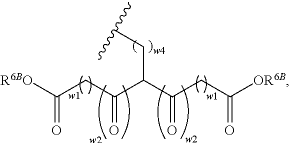

wherein w1, w2, w4, and R^{6B} are as defined herein. In certain embodiments, R¹ is of formula:

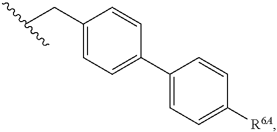

and at least one instance of R^{6A} is:

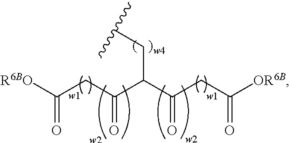

wherein w1, w2, w4, and R^{6B} are as defined herein. In certain embodiments, at least one instance of R^{6A} is:

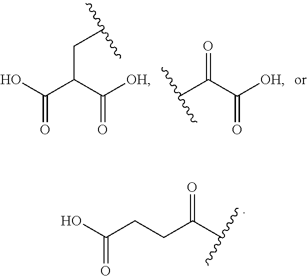 [SMILES: *CC(C(=O)O)C(=O)O ; *C(=O)C(=O)O ; *C(=O)CCC(=O)O]

In certain embodiments, at least one instance of R^{6A} is:

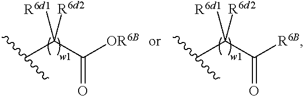

wherein w1 is 0, 1, 2, or 3; at least one instance of R^{6d1} and R^{6d2} is hydrogen, halogen, substituted or unsubstituted acyl, substituted or unsubstituted alkyl, —NH₂, —OH, or —O(substituted or unsubstituted alkyl); and R^{6B} is hydrogen or substituted or unsubstituted alkyl. In certain embodiments, at least one instance of R^{6A} is:

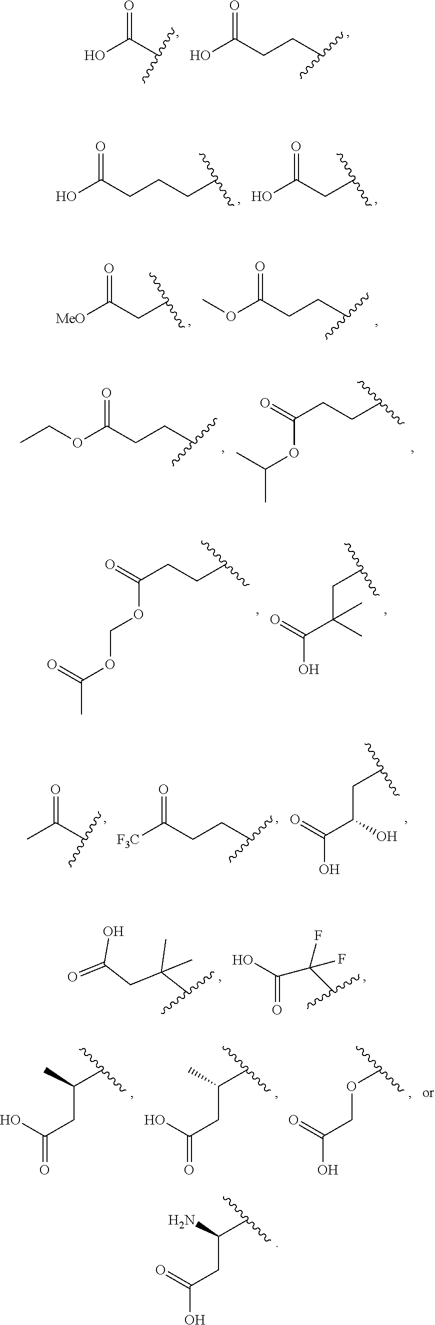

In certain embodiments, at least one instance of R^{6A} is:

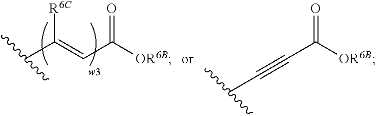

wherein: w3 is 1, 2, or 3; R^{6B} is hydrogen or substituted or unsubstituted alkyl; and R^{6C} is hydrogen, halogen, substituted or unsubstituted acyl, substituted or unsubstituted alkyl, —NH₂, —OH, or —O(substituted or unsubstituted alkyl). In certain embodiments, at least one instance of R^{6A} is:

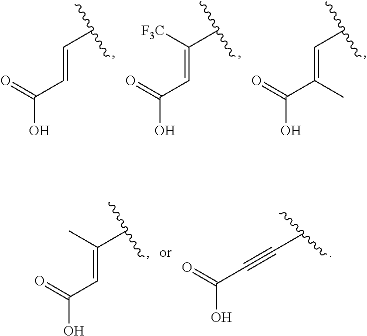

In certain embodiments, at least one instance of R^{6A} is:

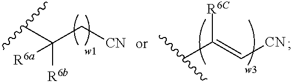

wherein: w1 is 0, 1, 2, 3, 4, 5, or 6; w3 is 1, 2, or 3; R^{6a} and R^{6b} are each independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN; or optionally, R^{6a} and R^{6b} are joined together with the intervening atoms to form an optionally substituted carbocycyl group; and each instance of R^{6C} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN. In certain embodiments, at least one instance of R^{6A} is

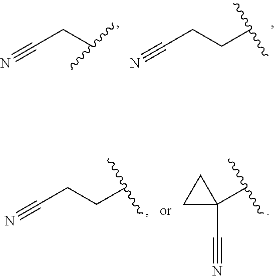

In certain embodiments, at least one instance of R^{6A} is

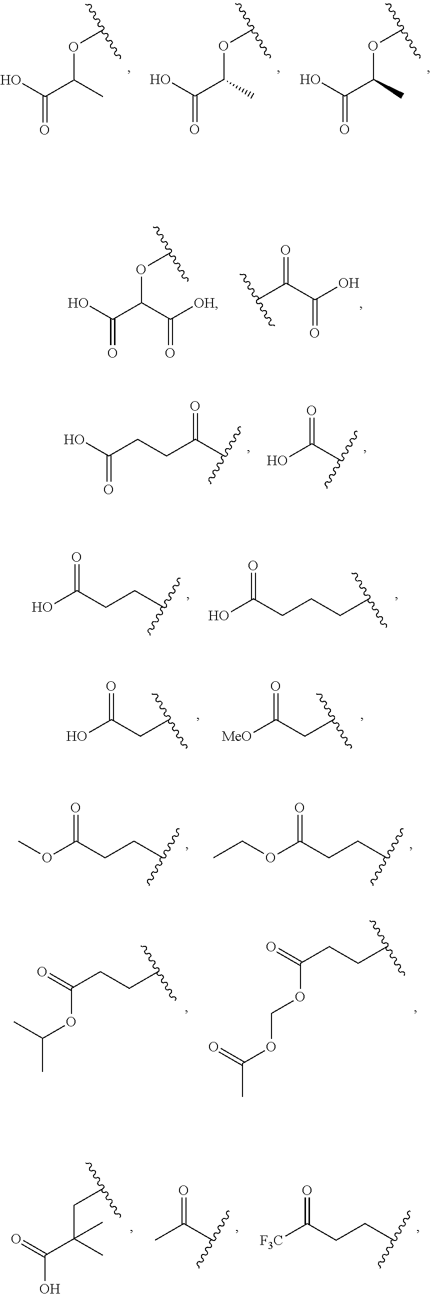

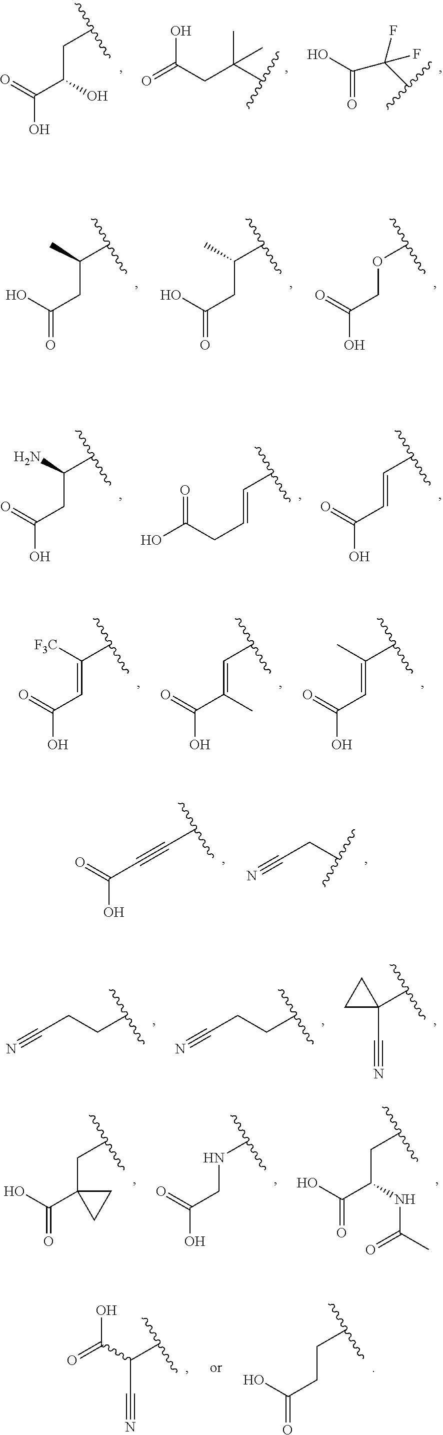

In certain embodiments, at least one instance of R^{6A} is substituted or unsubstituted methyl. In certain embodiments, at least one instance of R^{6A} is substituted or unsubstituted ethyl. In certain embodiments, at least one instance of R^{6A} is

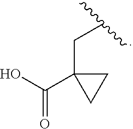 [SMILES: *CC1(C(=O)O)CC1]

In certain embodiments, at least one instance of R^{6A} is substituted or unsubstituted propyl. In certain embodiments, at least one instance of R^{6A} is substituted or unsubstituted alkenyl (e.g., substituted or unsubstituted C₂₋₆ alkenyl). In certain embodiments, at least one instance of R^{6A} is

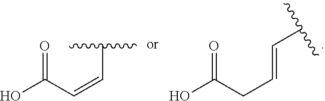 [SMILES: */C=C\C(=O)O ; */C=C/CC(=O)O]

In certain embodiments, at least one instance of R^{6A} is substituted or unsubstituted alkynyl (e.g., substituted or unsubstituted C₂₋₆ alkynyl). In certain embodiments, at least one instance of R^{6A} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{6A} is substituted or unsubstituted cyclopropyl. In certain embodiments, at least one instance of R^{6A} is

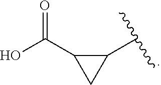 [SMILES: *C1CC1C(=O)O]

In certain embodiments, at least one instance of R^{6A} is

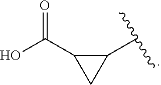 [SMILES: *C1CC1C(=O)O]

In certain embodiments, at least one instance of R^{6A} is

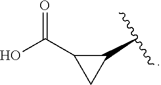

In certain embodiments, at least one instance of R^{6A} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{6A} is optionally substituted 2,3-dihydrobenzofuran. In certain embodiments, at least one instance of R^{6A} is

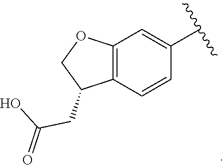 [SMILES: *c1ccc2c(c1)OC[C@H]2CC(=O)O]

In certain embodiments, at least one instance of R^{6A} is substituted or unsubstituted aryl (e.g., substituted or unsubstituted, 6- to 10-membered aryl). In certain embodiments, at least one instance of R^{6A} is optionally substituted benzyl. In certain embodiments, at least one instance of R^{6A} is benzyl. In certain embodiments, at least one instance of R^{6A} is substituted or unsubstituted phenyl. In certain embodiments, at least one instance of R^{6A} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{6A} is optionally substituted tetrazole or optionally substituted benzofuran. In certain embodiments, at least one instance of R^{6A} is

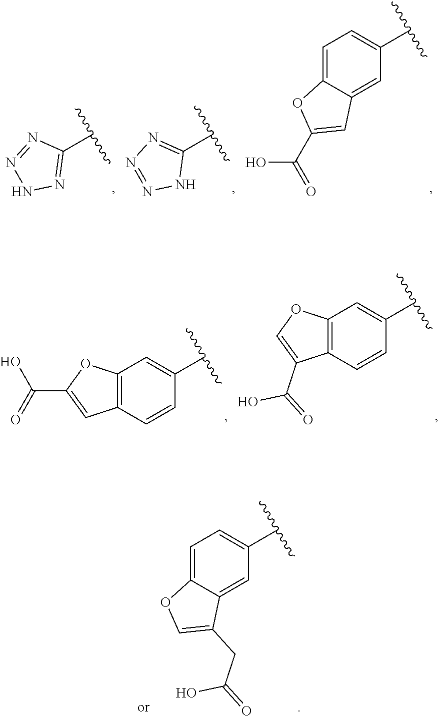 [SMILES: *c1nn[nH]n1 ; *c1nnn[nH]1 ; *c1ccc2oc(C(=O)O)cc2c1 ; *c1ccc2cc(C(=O)O)oc2c1 ; *c1ccc2c(C(=O)O)coc2c1 ; *c1ccc2occ(CC(=O)O)c2c1]

In certain embodiments, R¹ is of formula:

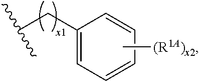

wherein at least one instance of R^{1A} is

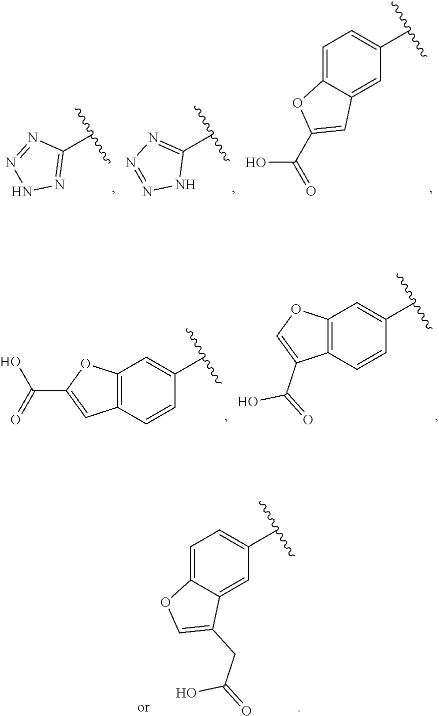 [SMILES: *c1nn[nH]n1 ; *c1nnn[nH]1 ; *c1ccc2oc(C(=O)O)cc2c1 ; *c1ccc2cc(C(=O)O)oc2c1 ; *c1ccc2c(C(=O)O)coc2c1 ; *c1ccc2occ(CC(=O)O)c2c1]

In certain embodiments, at least one instance of R^{1A} is optionally substituted 2,3-dihydrobenzofuran. In certain embodiments, at least one instance of R^{1A} is

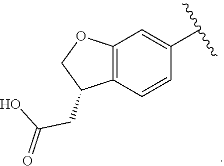 [SMILES: *c1ccc2c(c1)OC[C@H]2CC(=O)O]

In certain embodiments, at least one instance of R^{6A} is —OR^{c1} (e.g., —OH or —OMe). In certain embodiments, at least one instance of R^{6A} is —O(optionally substituted alkyl). In certain embodiments, at least one instance of R^{6A} is —O(optionally substituted C₁₋₆ alkyl). In certain embodiments, at least one instance of R^{6A} is

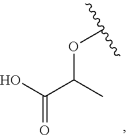 [SMILES: *OC(C)C(=O)O]

for example,

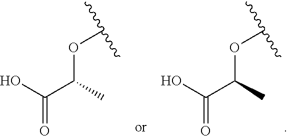

In certain embodiments, at least one instance of R^{6A} is —NO₂. In certain embodiments, at least one instance of R^{6A} is —N(R^{c2})₂ (e.g., —NMe₂). In certain embodiments, at least one instance of R^{6A} is —N(R^{c2})₂, wherein at least one instance of R^{c2} is hydrogen. In certain embodiments, at least one instance of R^{6A} is —N(R^{c2})₂, wherein at least one instance of R^{c2} is hydrogen and the other instance of R^{c2} is optionally substituted C₁₋₆ alkyl. In certain embodiments, at least one instance of R^{6A} is

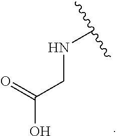 [SMILES: *NCC(=O)O]

In certain embodiments, at least one instance of R^{6A} is —SR^{c1} (e.g., —SMe). In certain embodiments, at least one instance of R^{6A} is —CN. In certain embodiments, at least one instance of R^{6A} is —B(OR^{c1})₂ (e.g., —B(OH)₂, —B(OMe)₂). In certain embodiments, at least one instance of R^{6A} is —B(OH)₂. In certain embodiments, at least one instance of R^{6A} is —B(O(optionally substituted alkyl))₂. In certain embodiments, at least one instance of R^{6A} is —B(O(optionally substituted C₁₋₆ alkyl))₂. In certain embodiments, at least one instance of R^{6A} is —SCN.

In certain embodiments, the compound is of formula:

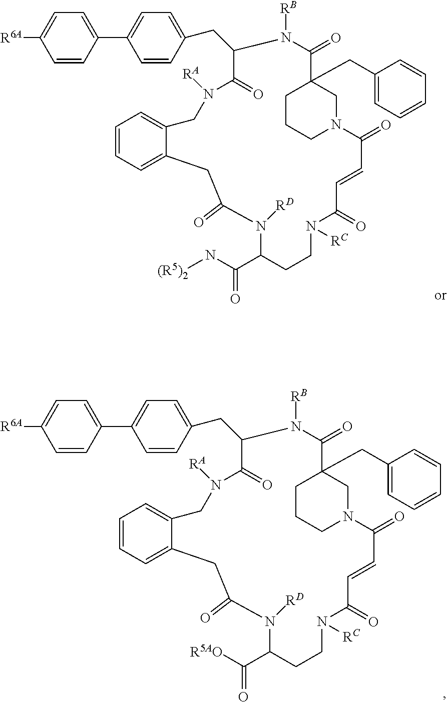

or a pharmaceutically acceptable salt, solvate, hydrate, stereoisomer, polymorph, tautomer, isotopically enriched form, or prodrug thereof, wherein: R^{6A} is substituted or unsubstituted acyl, —C(═O)N(R^{6c})₂,

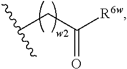

—NO₂, —SO₂R^{c1}, —SO₂N(R^{6c})₂, —B(OR^{c1})₂, or —OR^{c1}; each instance of R^{6c} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group; R^{6d} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or an oxygen protecting group; and R^{6w} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group; and w2 is 0, 1, 2, 3, 4, 5, or 6.

In certain embodiments, the compound is of formula:

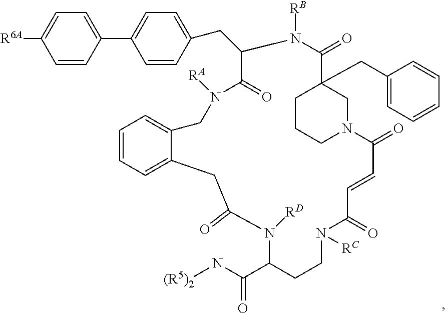

or a pharmaceutically acceptable salt, solvate, hydrate, stereoisomer, polymorph, tautomer, isotopically enriched form, or prodrug thereof, wherein: R^{6A} is substituted or unsubstituted acyl, —C(═O)N(R^{6c})₂,

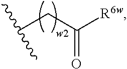

—NO₂, —SO₂R^{c1}, —SO₂N(R^{6c})₂, —B(OH)₂, or —OR^{c1}; each instance of R^{6c} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group; R^{6d} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or an oxygen protecting group; R^{6w} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group; and w2 is 0, 1, 2, 3, 4, 5, or 6.

In certain embodiments, R^{6A} is substituted or unsubstituted acyl, —C(═O)N(R)₂,

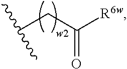

—NO₂, —SO₂R^{c1}, —SO₂N(R^{6c})₂, —B(OR^{c1})₂, or —OR^{c1}, wherein R^{6c} and R^{6c1} are as described herein. In certain embodiments, R^{6A} is substituted or unsubstituted acyl,

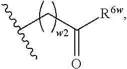

—NO₂, —SO₂R^{c1}, —B(OR^{c1})₂, or —OR^{c1}, wherein R^{6a} and R^{6c} are as described herein. In certain embodiments, at least one instance of R^{6c} is hydrogen. In certain embodiments, R^{6A} is substituted or unsubstituted acyl, —C(═O)N(R^{6c})₂,

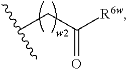

—NO₂, —SO₂R^{c1}, —SO₂N(R^{6c})₂, —B(OH)₂, or —OR^{c1}, wherein R^{6c} is as described herein. In certain embodiments, at least one instance of R^{6c} is hydrogen. In certain embodiments, at least one instance of R^{6A} is substituted or unsubstituted acyl (e.g., —C(═O)Me). In certain embodiments, R^{6A} is —C(═O)N(R^{6c})₂ (e.g., —C(═O)NH₂). In certain embodiments, R^{6A} is not —C(═O)N(R^{6c})₂ (e.g., is not —C(═O)NH₂). In certain embodiments, at least one instance of R^{6A} is

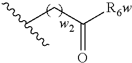

(e.g., —CH₂C(═O)OMe). In certain embodiments, at least one instance of R^{6A} is

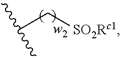

w2, w2 is 0, 1, 2, 3, 4, 5, or 6; and R^{c1} is halogen, optionally substituted acyl, optionally substituted alkyl, —OH, —O(optionally substituted alkyl), or —NH₂. In certain embodiments, at least one instance of R^{6A} is

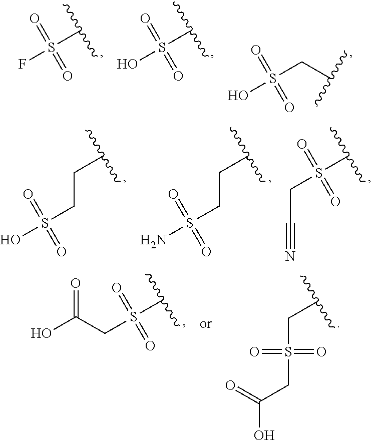

In certain embodiments, at least one instance of w2 is 0. In certain embodiments, at least one instance of w2 is 1. In certain embodiments, at least one instance of w2 is 2. In certain embodiments, at least one instance of w2 is 3. In certain embodiments, at least one instance of w2 is 4. In certain embodiments, at least one instance of w2 is 5. In certain embodiments, at least one instance of w2 is 6. In certain embodiments, at least one instance of R^{6w} is hydrogen. In certain embodiments, at least one instance of R^{6w} is substituted or unsubstituted acyl (e.g., —C(═O)Me). In certain embodiments, R^{6w} is substituted or unsubstituted alkyl (e.g., substituted or unsubstituted C₁₋₆ alkyl). In certain embodiments, R^{6w} is substituted or unsubstituted alkenyl (e.g., substituted or unsubstituted C₂₋₆ alkenyl). In certain embodiments, R^{6w} is substituted or unsubstituted alkynyl (e.g., substituted or unsubstituted C₂₋₆ alkynyl). In certain embodiments, R^{6w} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{6w} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{6w} is substituted or unsubstituted aryl (e.g., substituted or unsubstituted, 6- to 10-membered aryl). In certain embodiments, R^{6w} is optionally substituted benzyl. In certain embodiments, R^{6w} is benzyl. In certain embodiments, R^{6w} is substituted or unsubstituted phenyl. In certain embodiments, R^{6w} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9 to 10-membered, bicyclic heteroaryl, wherein one, two, three, or four atoms in the heteroaryl ring system are independently nitrogen, oxygen, or sulfur). In certain embodiments, R^{6w} is —OR^{6d} (e.g., —OMe). In certain embodiments, R^{6w} is an oxygen protecting group.

In certain embodiments, at least one instance of R^{6A} is —SO₂R^{c1} (e.g., —SO₂Me). In certain embodiments, at least one instance of R^{6A} is —SO₂N(R^{6c})₂ (e.g., —SO₂NHMe). In certain embodiments, at least one instance of R^{6A} is —B(OH)₂. In certain embodiments, at least one instance of R^{6A} is —B(O(optionally substituted alkyl))₂. In certain embodiments, at least one instance of R^{6A} is —B(O(optionally substituted C₁₋₆ alkyl))₂. In certain embodiments, at least one instance of R^{6A} is —OR^{c1} (e.g., —OMe). In certain embodiments, at least one instance of R^{6A} is —SO₂R^{c1}, and R^{c1} is halogen, —OH, or —O(optionally substituted alkyl). In certain embodiments, at least one instance of R^{6A} is —SO₂R^{c1}, and R^{c1} is halogen (e.g., F, Cl, Br, or I). In certain embodiments, at least one instance of R^{6A} is —SO₂F. In certain embodiments, at least one instance of R^{6A} is —SO₂(OH).

In certain embodiments, at least one instance of R^{6c} is substituted or unsubstituted alkyl (e.g., substituted or unsubstituted C₁₋₆ alkyl). In certain embodiments, at least one instance of R^{6c} is substituted or unsubstituted methyl. In certain embodiments, at least one instance of R^{6c} is substituted or unsubstituted ethyl. In certain embodiments, at least one instance of R^{6c} is substituted or unsubstituted alkenyl (e.g., substituted or unsubstituted C₂₋₆ alkenyl). In certain embodiments, at least one instance of R^{6c} is substituted or unsubstituted alkynyl (e.g., substituted or unsubstituted C₂₋₆ alkynyl). In certain embodiments, at least one instance of R^{6c} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{6c} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{6c} is substituted or unsubstituted aryl (e.g., substituted or unsubstituted, 6- to 10-membered aryl). In certain embodiments, at least one instance of R^{6c} is optionally substituted benzyl. In certain embodiments, at least one instance of R^{6c} is benzyl. In certain embodiments, at least one instance of R^{6c} is substituted or unsubstituted phenyl. In certain embodiments, at least one instance of R^{6c} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{6c} is —OR^{6d} (e.g., —OMe). In certain embodiments, at least one instance of R^{6c} is nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R¹ is of formula:

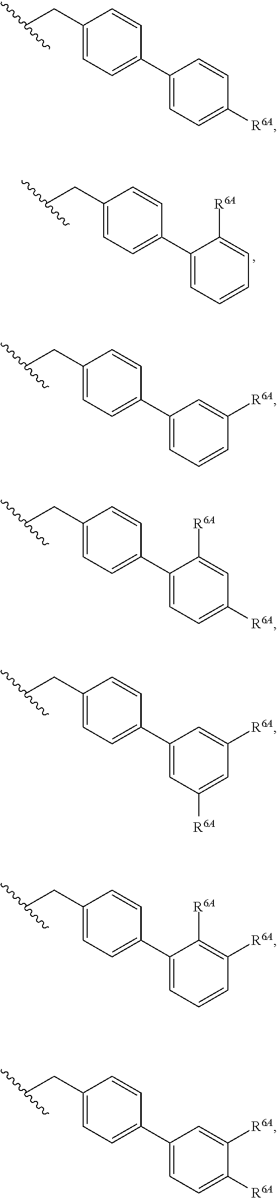

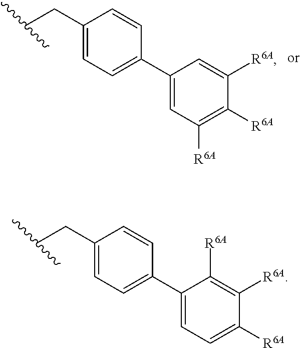

In certain embodiments, R¹ is of formula:

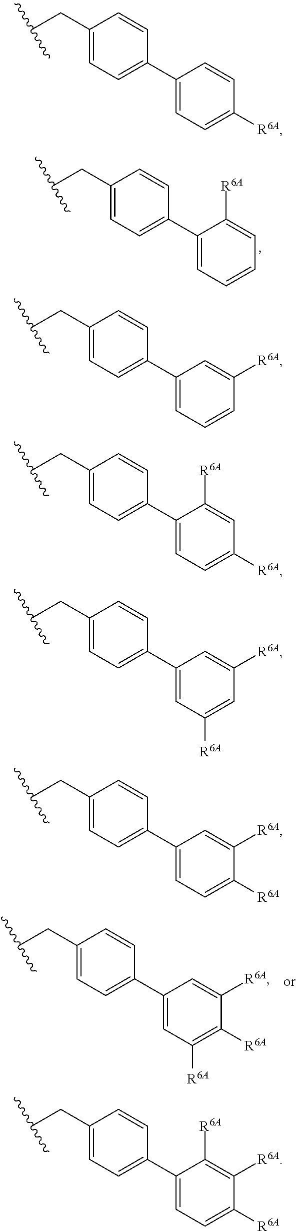

In certain embodiments, R¹ is of formula:

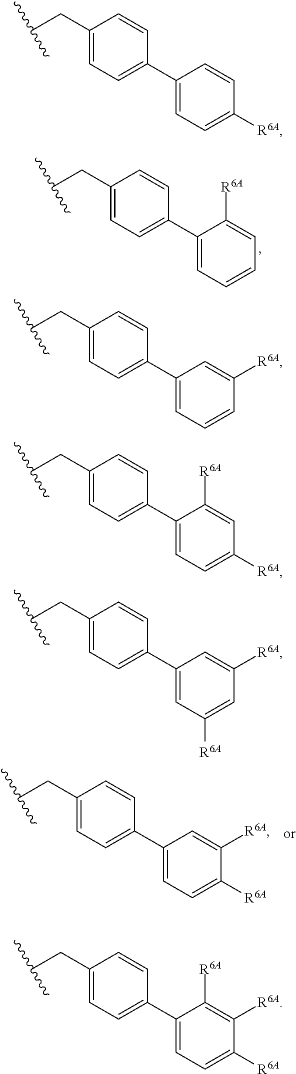

In certain embodiments, R¹ is of formula:

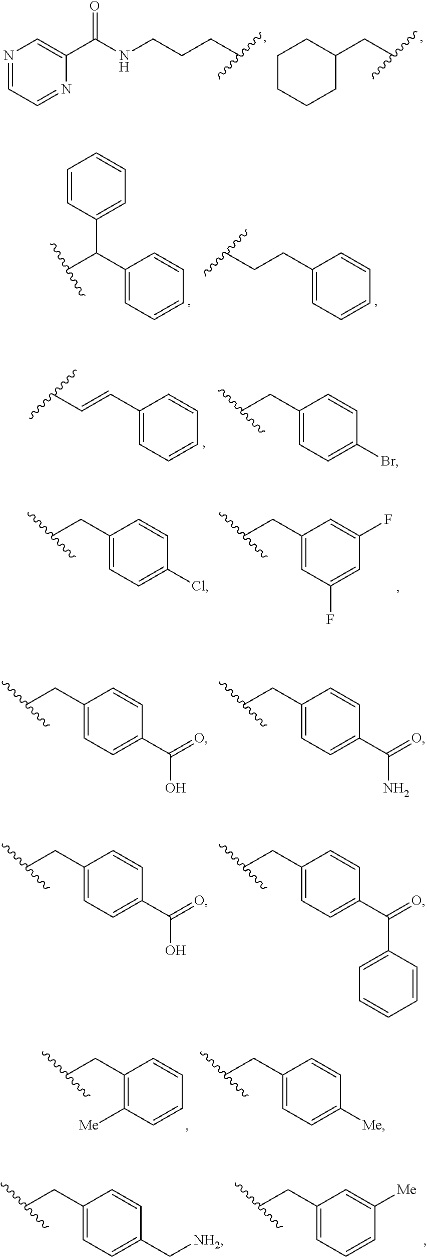

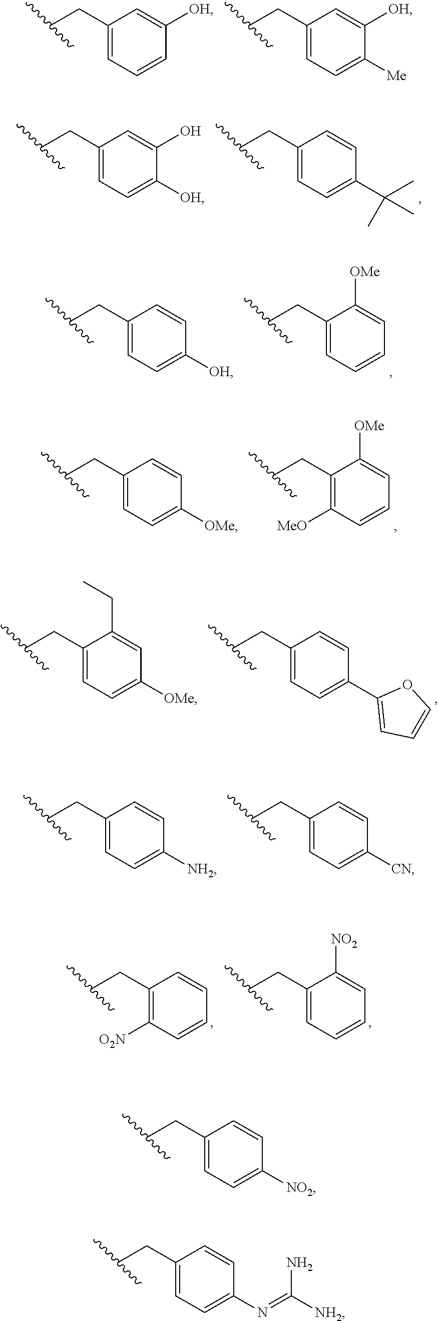

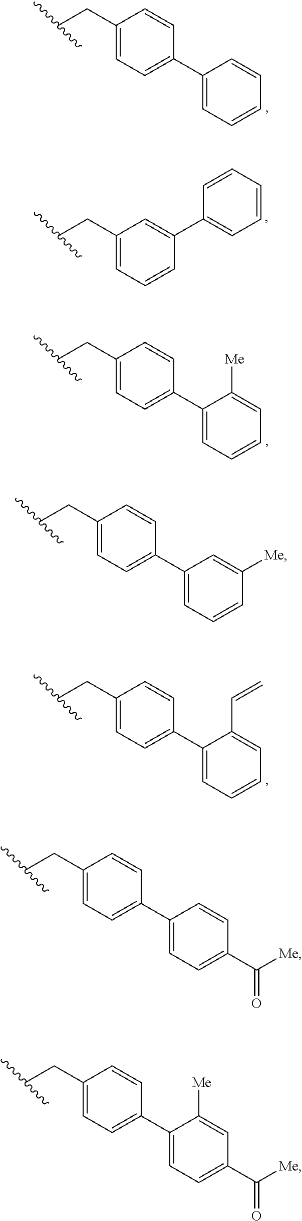

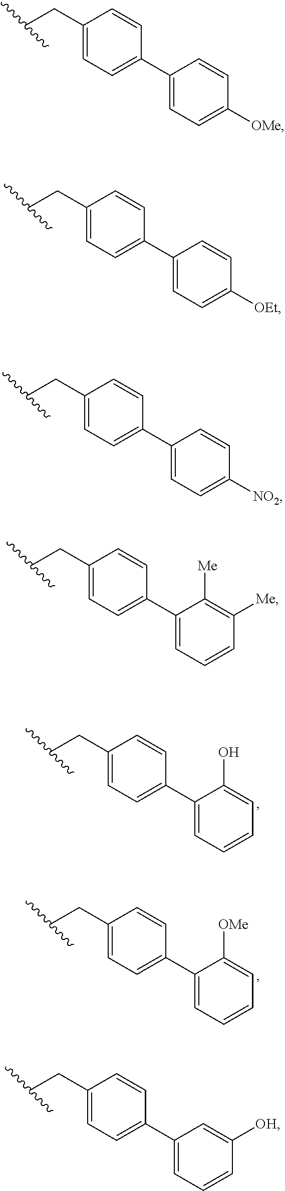

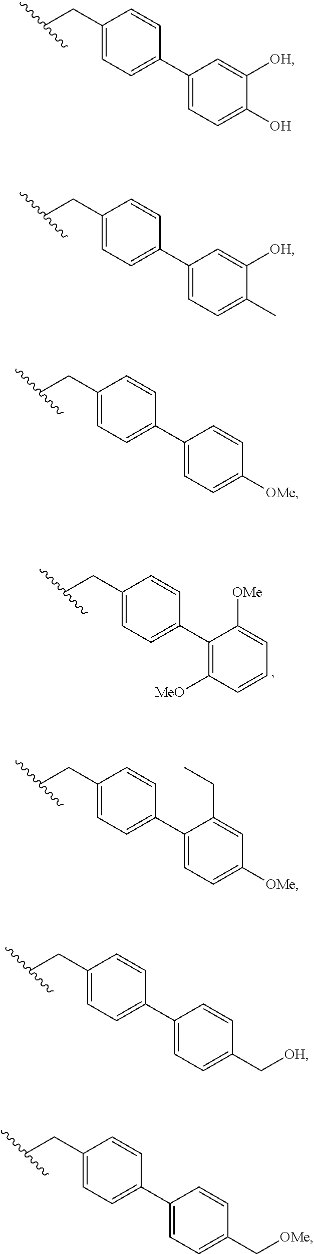

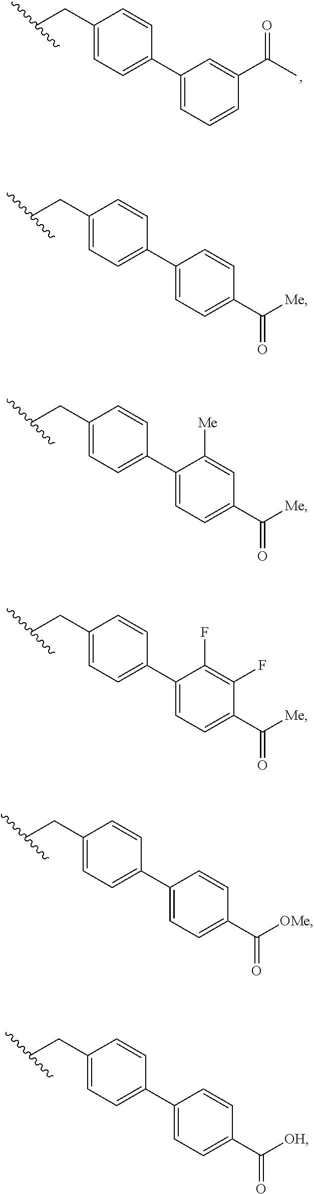 [SMILES: *Cc1ccc(-c2cccc(C(C)=O)c2)cc1 ; *Cc1ccc(-c2ccc(C(C)=O)cc2)cc1 ; *Cc1ccc(-c2ccc(C(C)=O)cc2C)cc1 ; *Cc1ccc(-c2ccc(C(C)=O)c(F)c2F)cc1 ; *Cc1ccc(-c2ccc(C(=O)OC)cc2)cc1 ; *Cc1ccc(-c2ccc(C(=O)O)cc2)cc1]

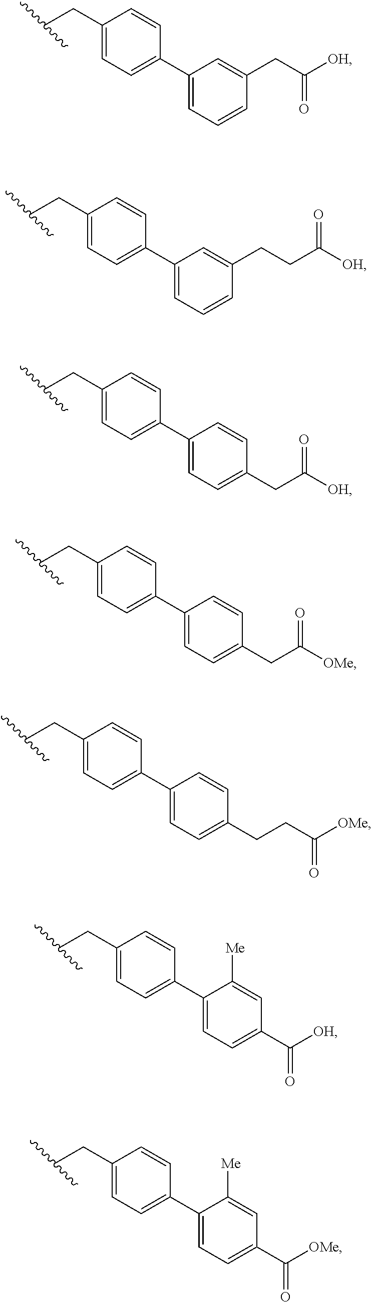

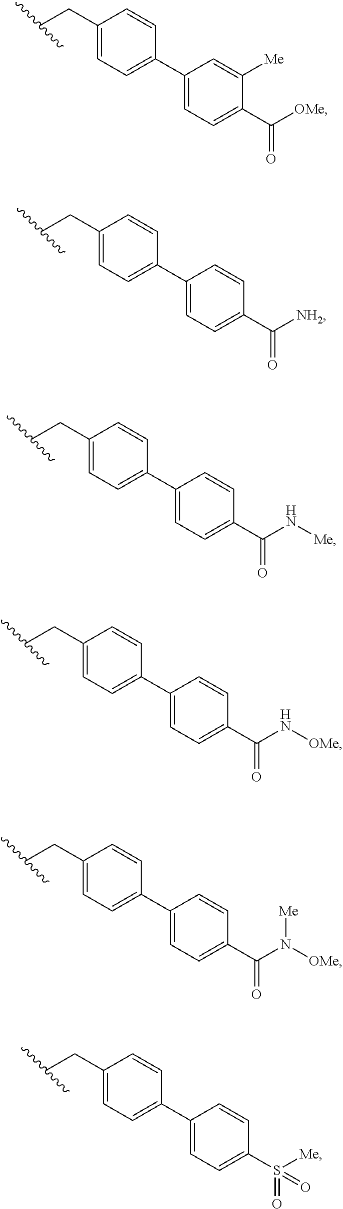 [SMILES: *Cc1ccc(-c2ccc(C(=O)OC)c(C)c2)cc1 ; *Cc1ccc(-c2ccc(C(N)=O)cc2)cc1 ; *Cc1ccc(-c2ccc(C(=O)NC)cc2)cc1 ; *Cc1ccc(-c2ccc(C(=O)NOC)cc2)cc1 ; *Cc1ccc(-c2ccc(C(=O)N(C)OC)cc2)cc1 ; *Cc1ccc(-c2ccc(S(C)(=O)=O)cc2)cc1]

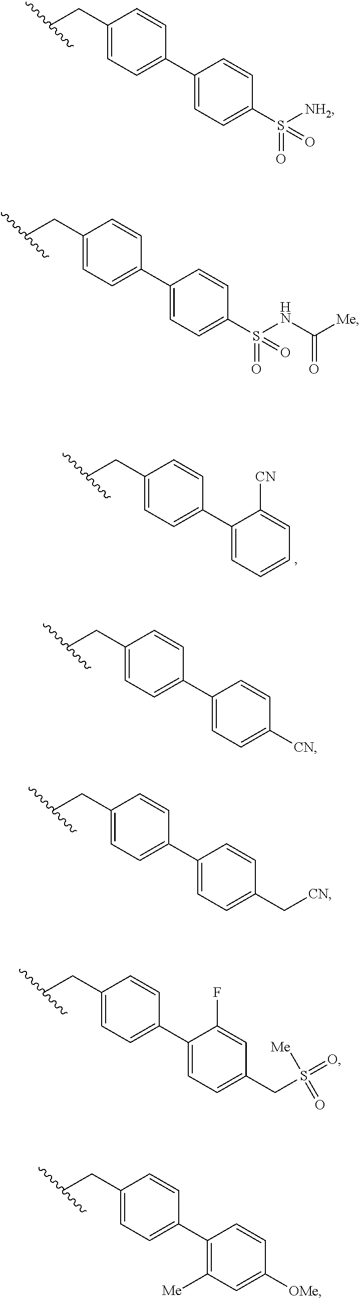

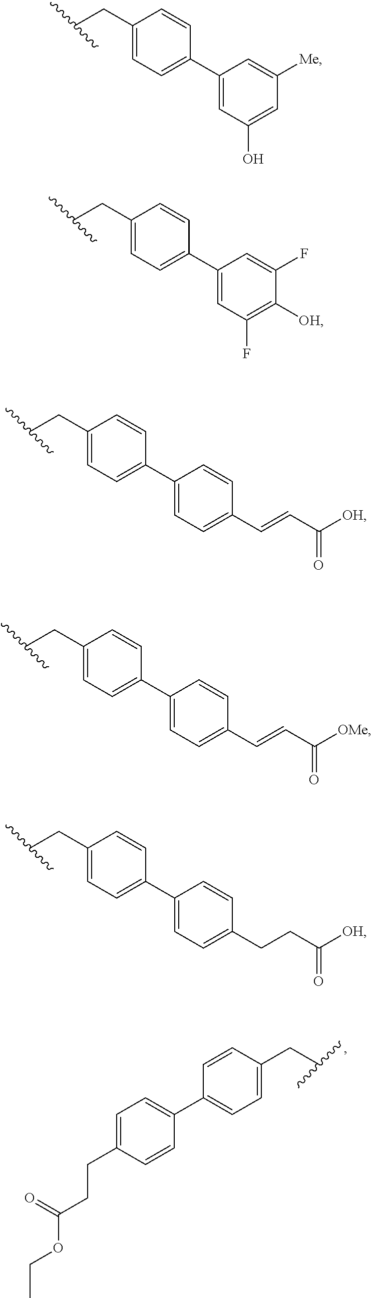 [SMILES: *Cc1ccc(-c2cc(C)cc(O)c2)cc1 ; *Cc1ccc(-c2cc(F)c(O)c(F)c2)cc1 ; *Cc1ccc(-c2ccc(/C=C/C(=O)O)cc2)cc1 ; *Cc1ccc(-c2ccc(/C=C/C(=O)OC)cc2)cc1 ; *Cc1ccc(-c2ccc(CCC(=O)O)cc2)cc1 ; *Cc1ccc(-c2ccc(CCC(=O)OCC)cc2)cc1]

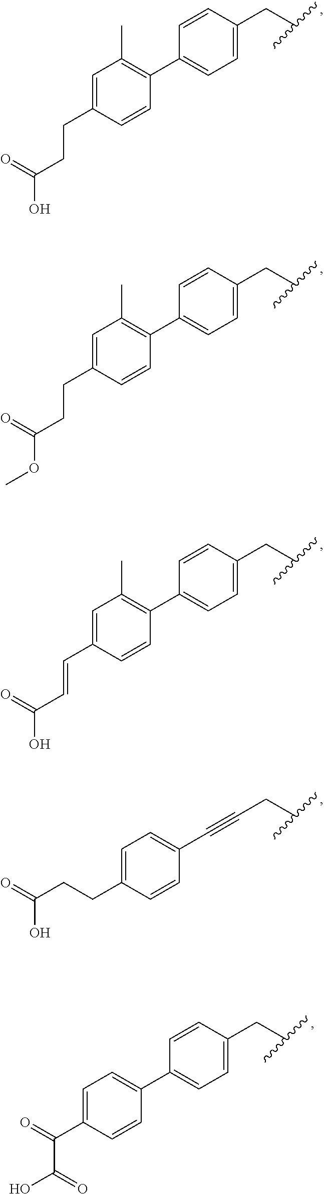 [SMILES: *Cc1ccc(-c2ccc(CCC(=O)O)cc2C)cc1 ; *Cc1ccc(-c2ccc(CCC(=O)OC)cc2C)cc1 ; *Cc1ccc(-c2ccc(/C=C/C(=O)O)cc2C)cc1 ; *CC#Cc1ccc(CCC(=O)O)cc1 ; *Cc1ccc(-c2ccc(C(=O)C(=O)O)cc2)cc1]

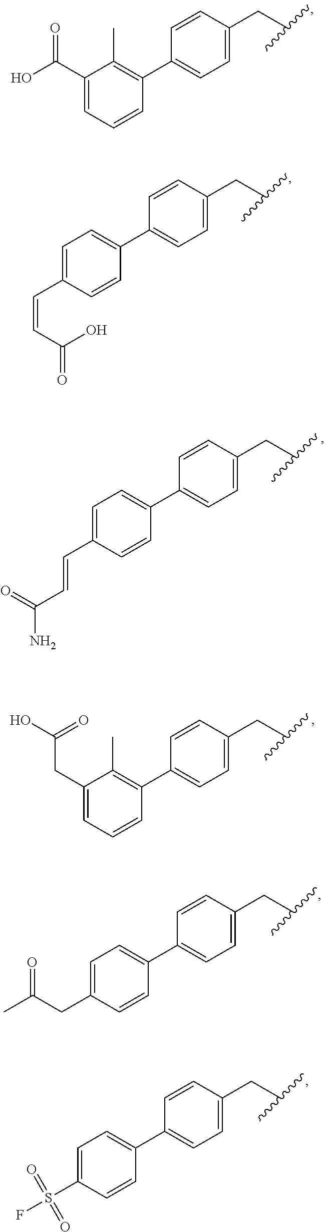 [SMILES: *Cc1ccc(-c2cccc(C(=O)O)c2C)cc1 ; *Cc1ccc(-c2ccc(/C=C\C(=O)O)cc2)cc1 ; *Cc1ccc(-c2ccc(/C=C/C(N)=O)cc2)cc1 ; *Cc1ccc(-c2cccc(CC(=O)O)c2C)cc1 ; *Cc1ccc(-c2ccc(CC(C)=O)cc2)cc1 ; *Cc1ccc(-c2ccc(S(=O)(=O)F)cc2)cc1]

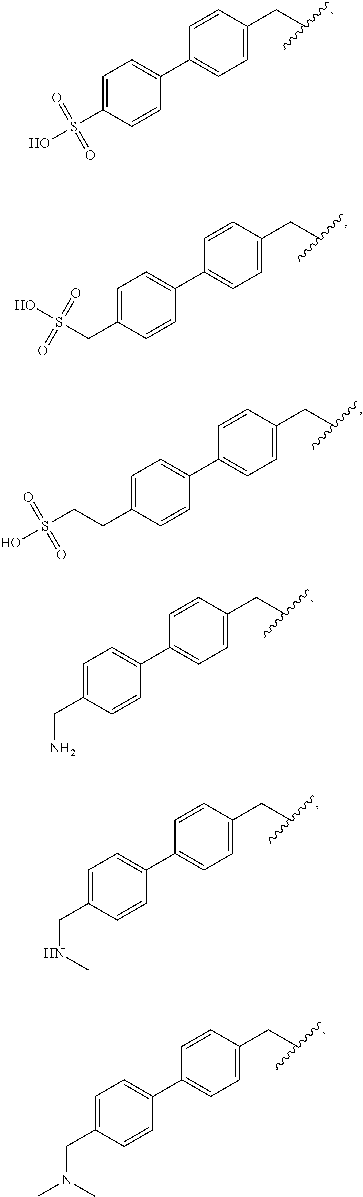 [SMILES: *Cc1ccc(-c2ccc(S(=O)(=O)O)cc2)cc1 ; *Cc1ccc(-c2ccc(CS(=O)(=O)O)cc2)cc1 ; *Cc1ccc(-c2ccc(CCS(=O)(=O)O)cc2)cc1 ; *Cc1ccc(-c2ccc(CN)cc2)cc1 ; *Cc1ccc(-c2ccc(CNC)cc2)cc1 ; *Cc1ccc(-c2ccc(CN(C)C)cc2)cc1]

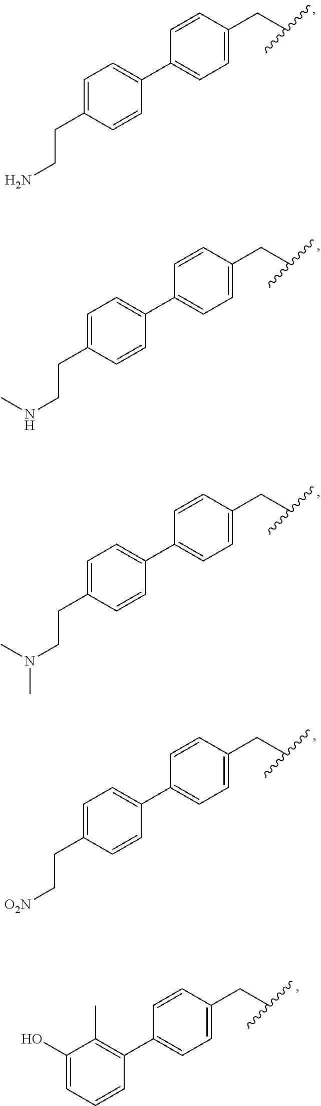 [SMILES: *Cc1ccc(-c2ccc(CCN)cc2)cc1 ; *Cc1ccc(-c2ccc(CCNC)cc2)cc1 ; *Cc1ccc(-c2ccc(CCN(C)C)cc2)cc1 ; *Cc1ccc(-c2ccc(CC[N+](=O)[O-])cc2)cc1 ; *Cc1ccc(-c2cccc(O)c2C)cc1]

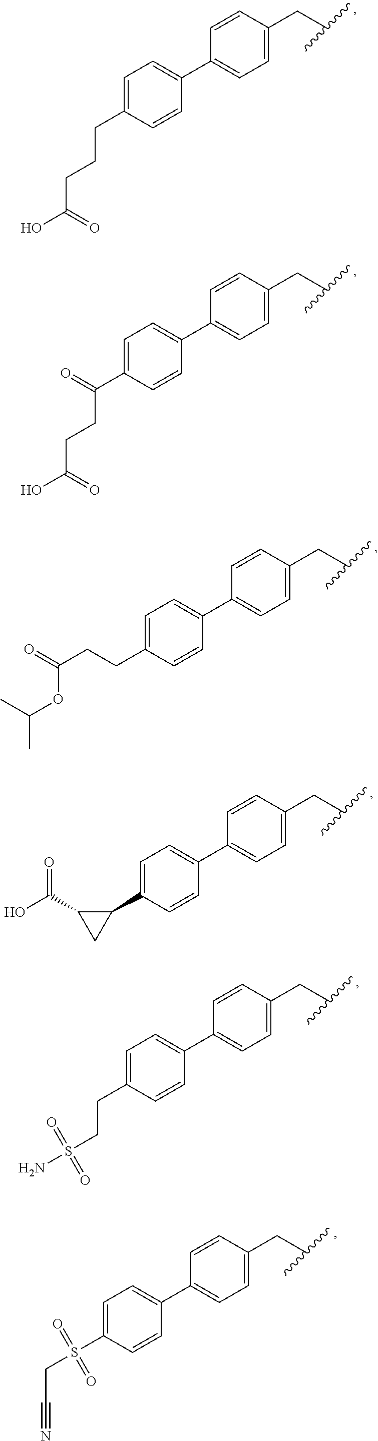 [SMILES: *Cc1ccc(-c2ccc(CCCC(=O)O)cc2)cc1 ; *Cc1ccc(-c2ccc(C(=O)CCC(=O)O)cc2)cc1 ; *Cc1ccc(-c2ccc(CCC(=O)OC(C)C)cc2)cc1 ; *Cc1ccc(-c2ccc([C@H]3C[C@@H]3C(=O)O)cc2)cc1 ; *Cc1ccc(-c2ccc(CCS(N)(=O)=O)cc2)cc1 ; *Cc1ccc(-c2ccc(S(=O)(=O)CC#N)cc2)cc1]

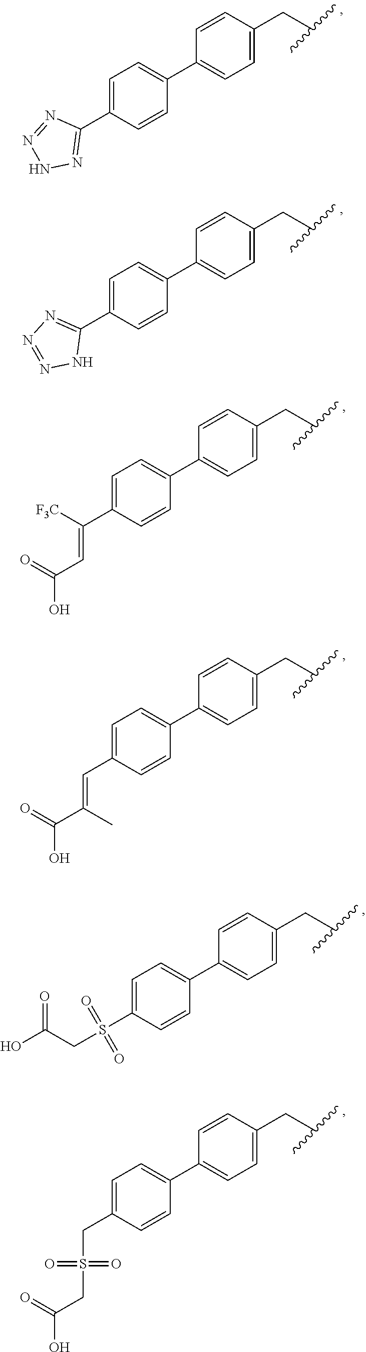 [SMILES: *Cc1ccc(-c2ccc(-c3nn[nH]n3)cc2)cc1 ; *Cc1ccc(-c2ccc(-c3nnn[nH]3)cc2)cc1 ; *Cc1ccc(-c2ccc(/C(=C/C(=O)O)C(F)(F)F)cc2)cc1 ; *Cc1ccc(-c2ccc(/C=C(\C)C(=O)O)cc2)cc1 ; *Cc1ccc(-c2ccc(S(=O)(=O)CC(=O)O)cc2)cc1 ; *Cc1ccc(-c2ccc(CS(=O)(=O)CC(=O)O)cc2)cc1]

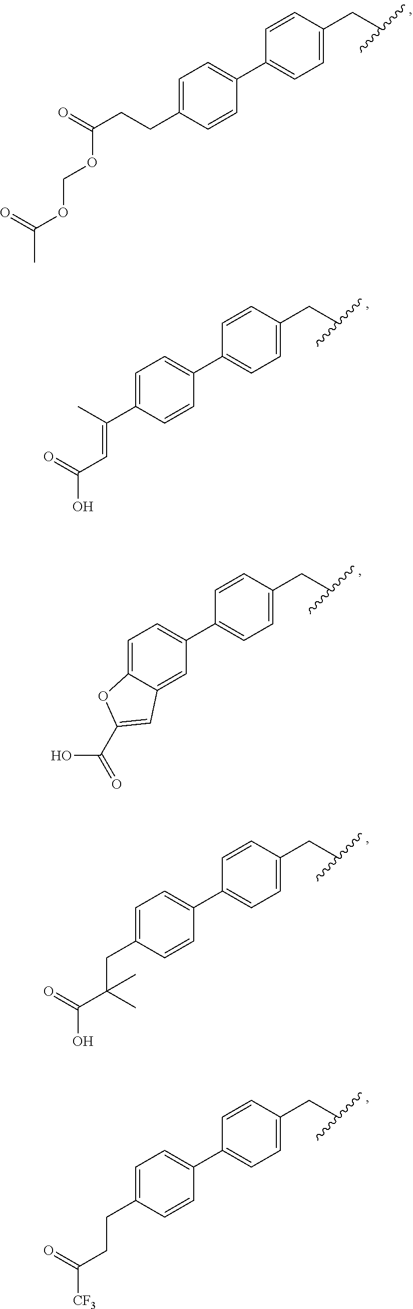 [SMILES: *Cc1ccc(-c2ccc(CCC(=O)OCOC(C)=O)cc2)cc1 ; *Cc1ccc(-c2ccc(/C(C)=C/C(=O)O)cc2)cc1 ; *Cc1ccc(-c2ccc3oc(C(=O)O)cc3c2)cc1 ; *Cc1ccc(-c2ccc(CC(C)(C)C(=O)O)cc2)cc1 ; *Cc1ccc(-c2ccc(CCC(=O)C(F)(F)F)cc2)cc1]

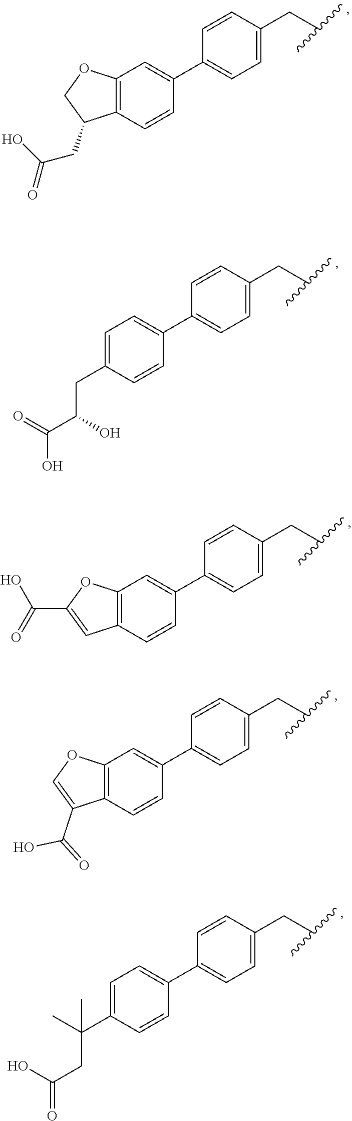 [SMILES: *Cc1ccc(-c2ccc3c(c2)OC[C@H]3CC(=O)O)cc1 ; *Cc1ccc(-c2ccc(C[C@H](O)C(=O)O)cc2)cc1 ; *Cc1ccc(-c2ccc3cc(C(=O)O)oc3c2)cc1 ; *Cc1ccc(-c2ccc3c(C(=O)O)coc3c2)cc1 ; *Cc1ccc(-c2ccc(C(C)(C)CC(=O)O)cc2)cc1]

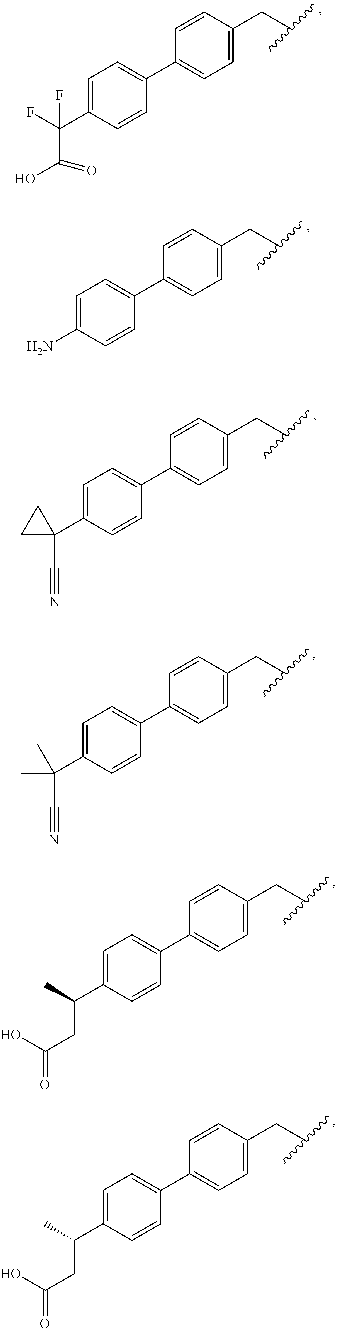 [SMILES: *Cc1ccc(-c2ccc(C(F)(F)C(=O)O)cc2)cc1 ; *Cc1ccc(-c2ccc(N)cc2)cc1 ; *Cc1ccc(-c2ccc(C3(C#N)CC3)cc2)cc1 ; *Cc1ccc(-c2ccc(C(C)(C)C#N)cc2)cc1 ; *Cc1ccc(-c2ccc([C@H](C)CC(=O)O)cc2)cc1 ; *Cc1ccc(-c2ccc([C@@H](C)CC(=O)O)cc2)cc1]

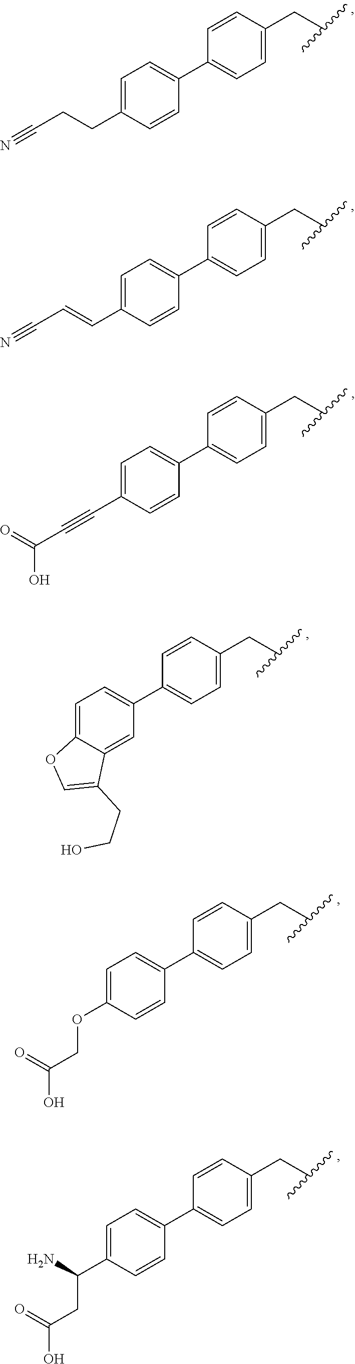 [SMILES: *Cc1ccc(-c2ccc(CCC#N)cc2)cc1 ; *Cc1ccc(-c2ccc(/C=C/C#N)cc2)cc1 ; *Cc1ccc(-c2ccc(C#CC(=O)O)cc2)cc1 ; *Cc1ccc(-c2ccc3occ(CCO)c3c2)cc1 ; *Cc1ccc(-c2ccc(OCC(=O)O)cc2)cc1 ; *Cc1ccc(-c2ccc([C@H](N)CC(=O)O)cc2)cc1]

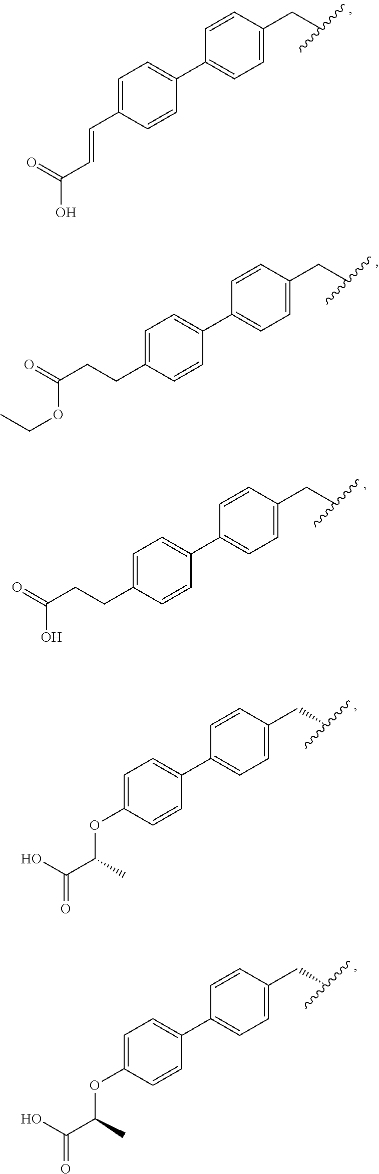

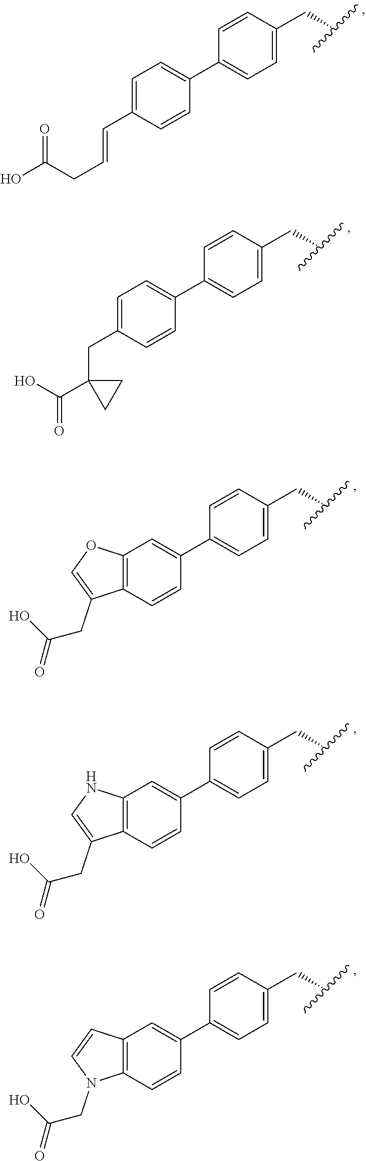

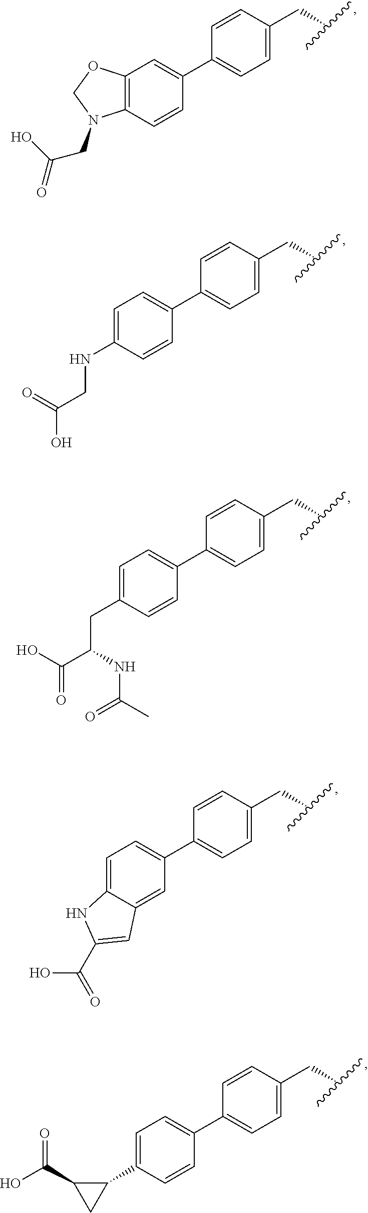

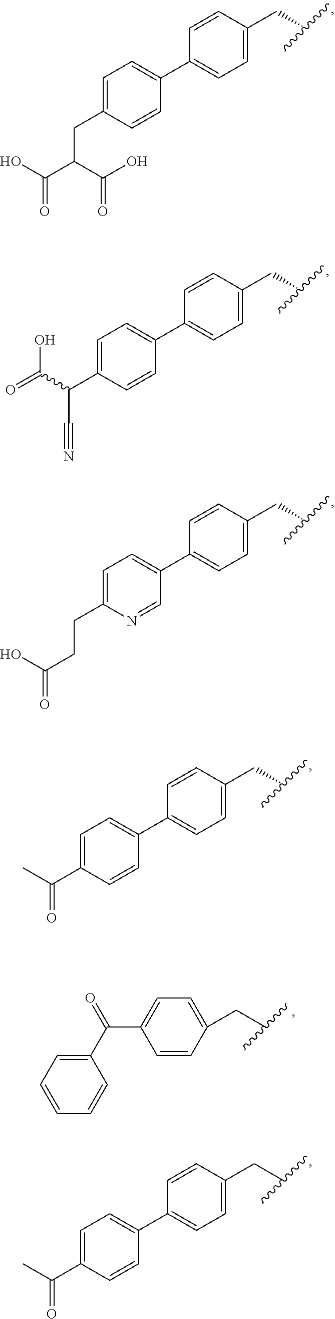

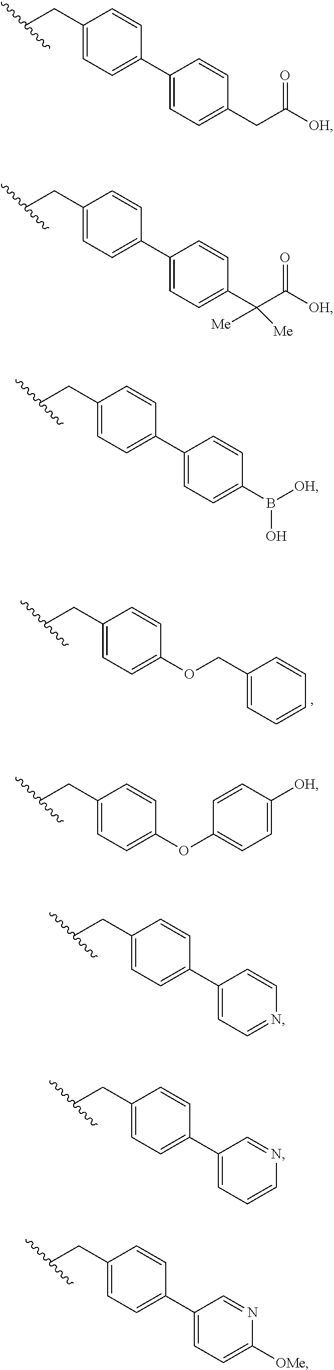

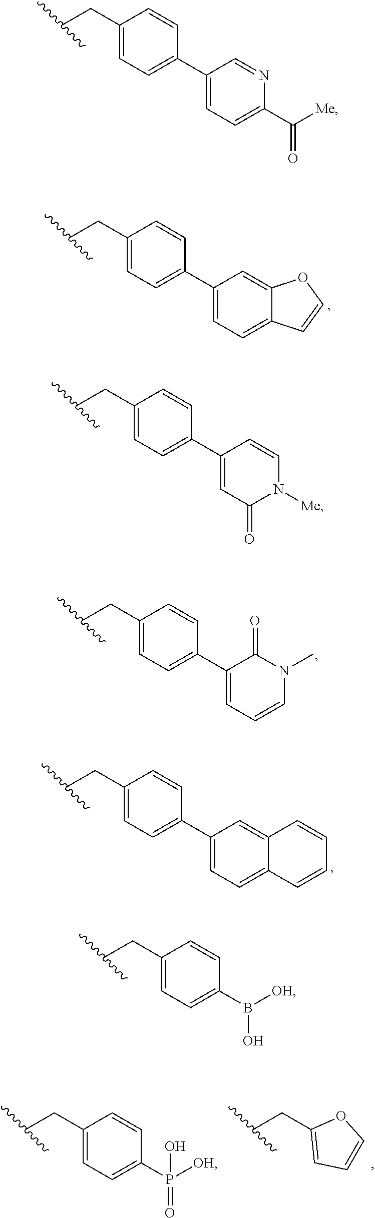

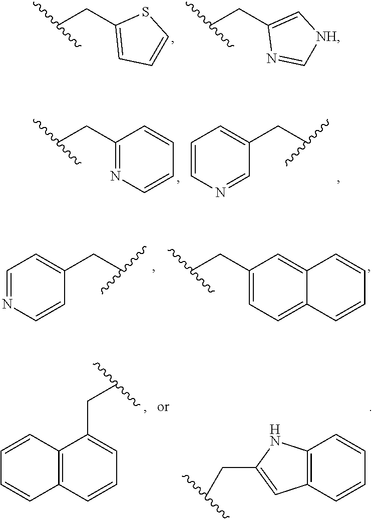

In certain embodiments, R¹ is of formula:

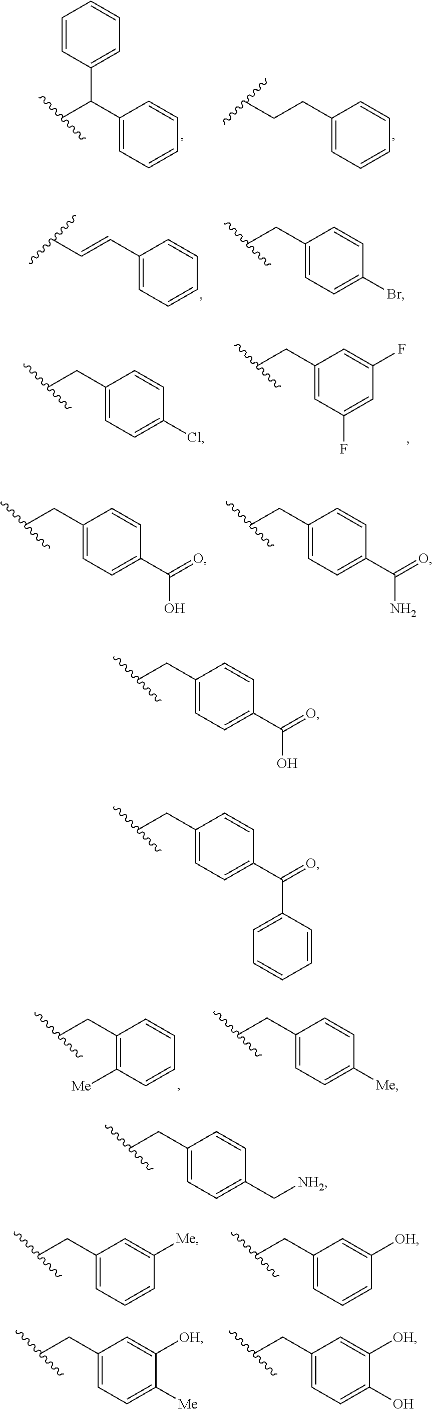

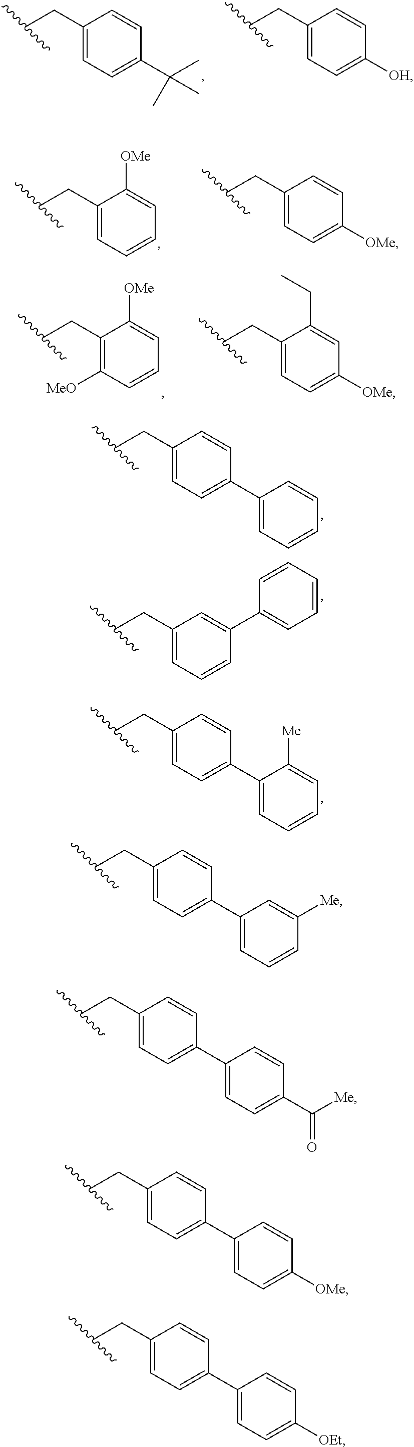

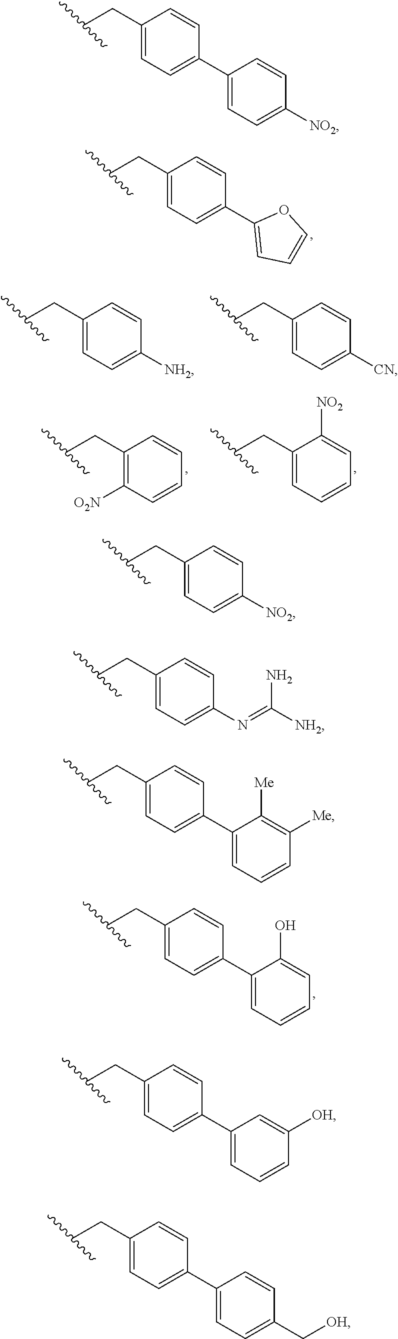

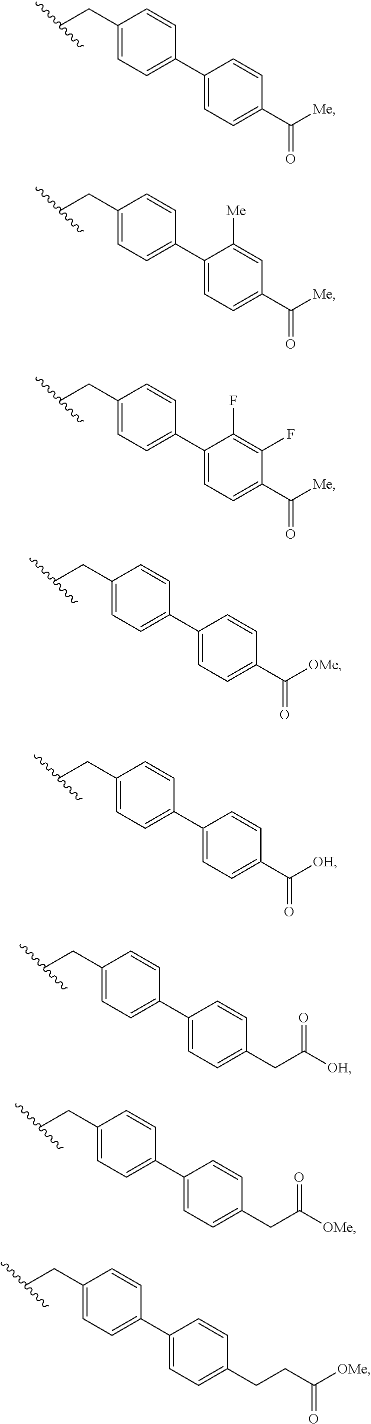

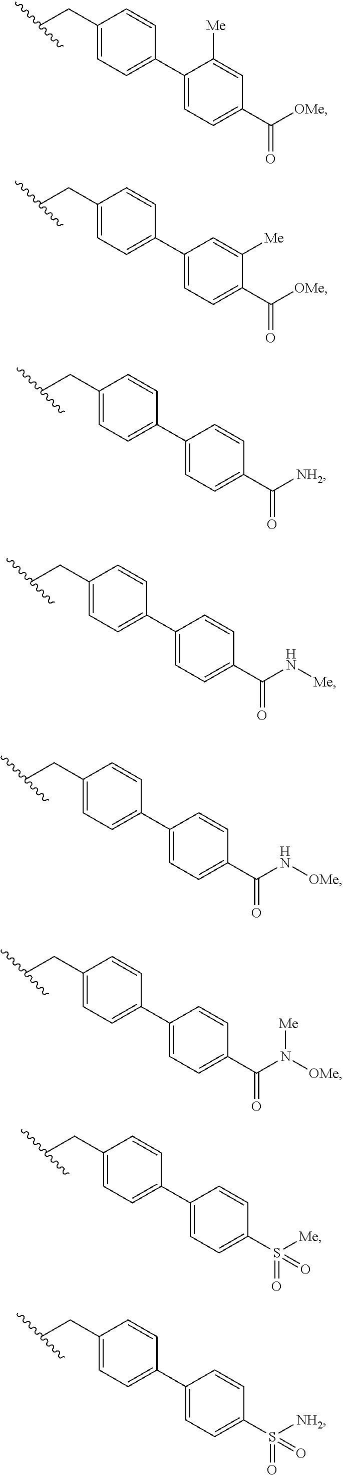

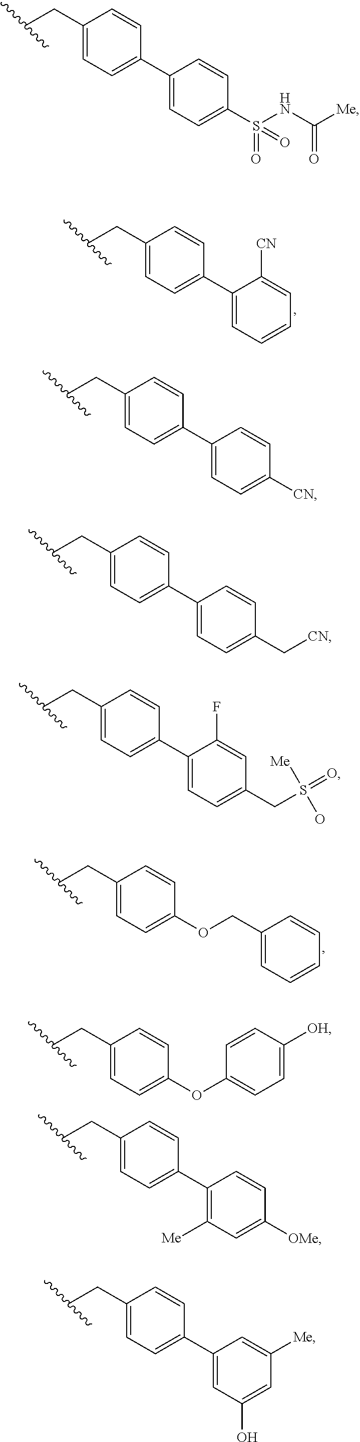

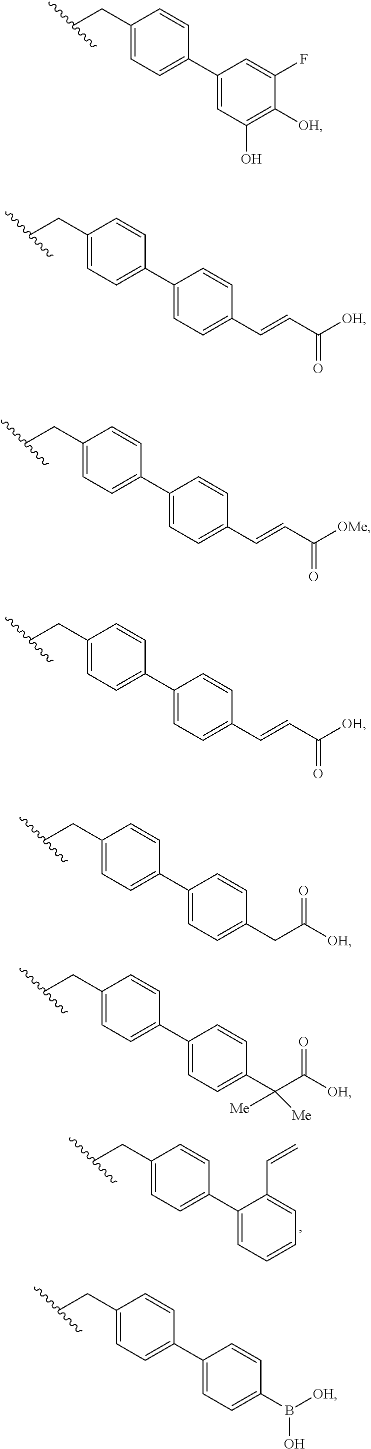

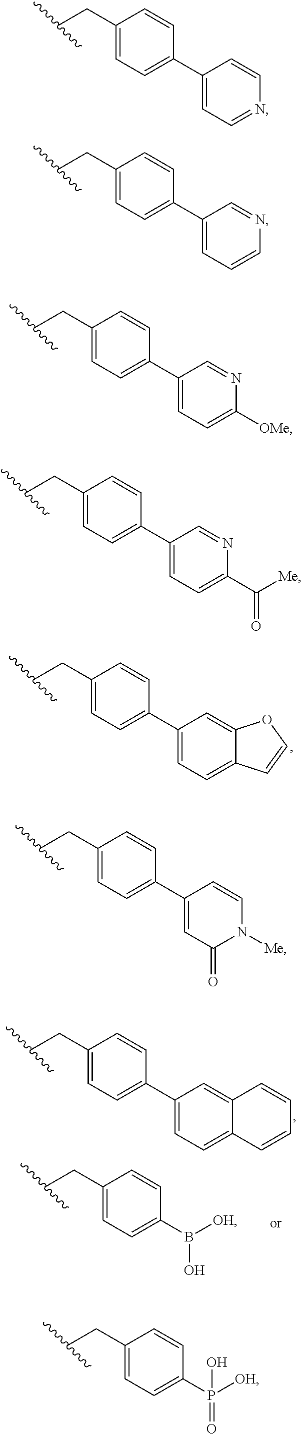

In certain embodiments, R¹ is of formula:

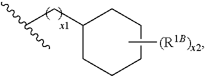

wherein: each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; and x2 is 0, 1, 2, 3, 4, 5, 6, 7, 8, 9, or 10. In certain embodiments, R¹ is of formula:

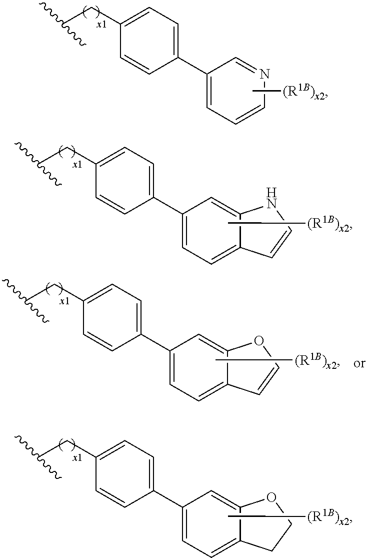

wherein each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or nitrogen protecting group when attached to a nitrogen atom; x1 is 0, 1, 2, 3, 4, 5, or 6; and x2 is 0, 1, 2, 3, 4, 5, or 6. In certain embodiments, R¹ is of formula:

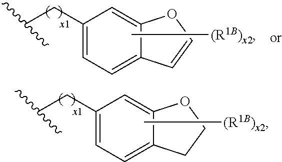

wherein each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or nitrogen protecting group when attached to a nitrogen atom; x1 is 0, 1, 2, 3, 4, 5, or 6; and x2 is 0, 1, 2, 3, 4, 5, or 6. In certain embodiments, at least one instance of R^{1A} is

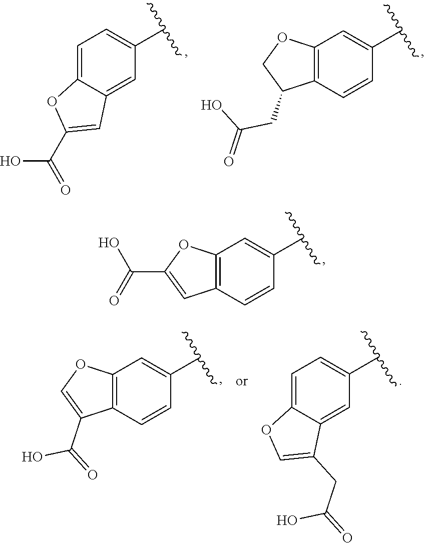 [SMILES: *c1ccc2oc(C(=O)O)cc2c1 ; *c1ccc2c(c1)OC[C@H]2CC(=O)O ; *c1ccc2cc(C(=O)O)oc2c1 ; *c1ccc2c(C(=O)O)coc2c1 ; *c1ccc2occ(CC(=O)O)c2c1]

In certain embodiments, at least one instance of R^{6A} is

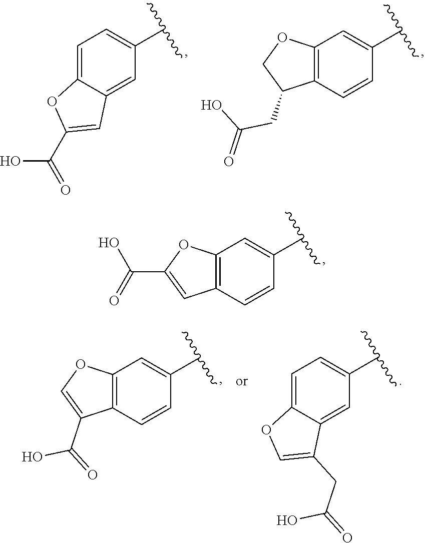 [SMILES: *c1ccc2oc(C(=O)O)cc2c1 ; *c1ccc2c(c1)OC[C@H]2CC(=O)O ; *c1ccc2cc(C(=O)O)oc2c1 ; *c1ccc2c(C(=O)O)coc2c1 ; *c1ccc2occ(CC(=O)O)c2c1]

In certain embodiments, R¹ is of formula:

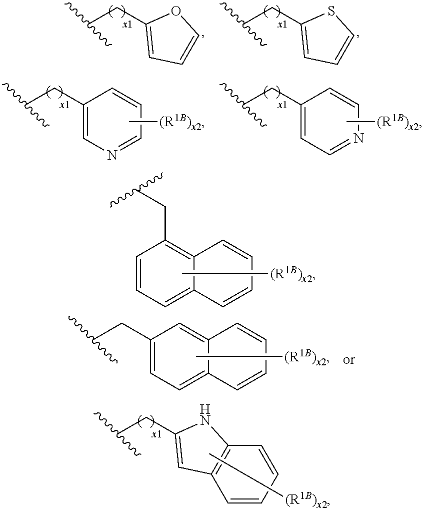

wherein each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, —SCN, or nitrogen protecting group when attached to a nitrogen atom; x1 is 0, 1, 2, 3, 4, 5, or 6; and x2 is 0, 1, 2, 3, 4, 5, or 6. In certain embodiments, R¹ is of formula:

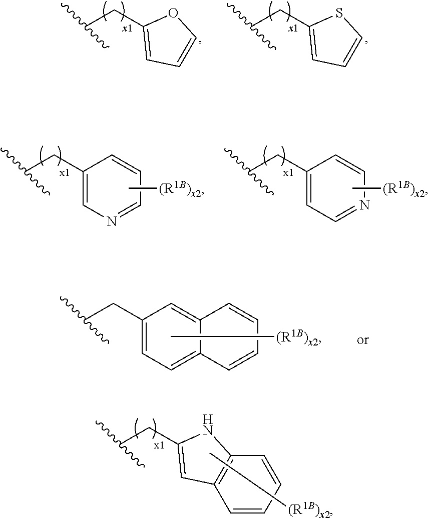

wherein: each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, —SCN, or nitrogen protecting group when attached to a nitrogen atom; x1 is 0, 1, 2, 3, 4, 5, or 6; and x2 is 0, 1, 2, 3, 4, 5, or 6. In certain embodiments, R¹ is of formula:

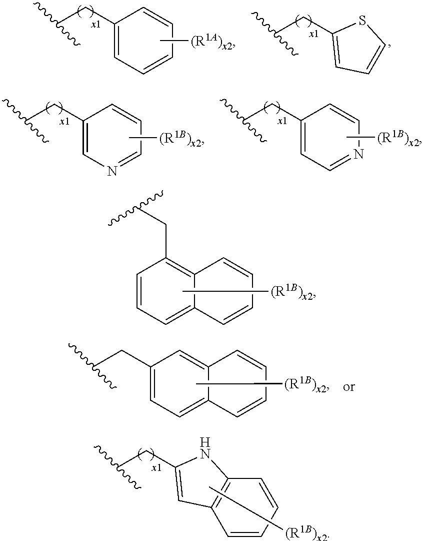

In certain embodiments, R¹ is of formula:

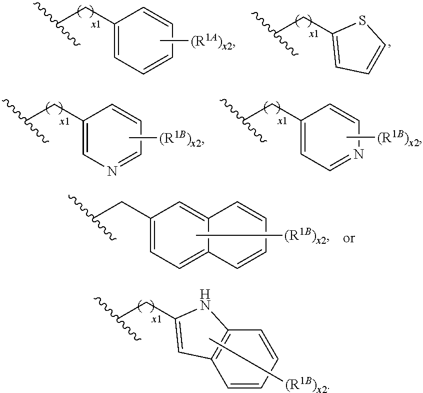

In certain embodiments, R¹ is of formula:

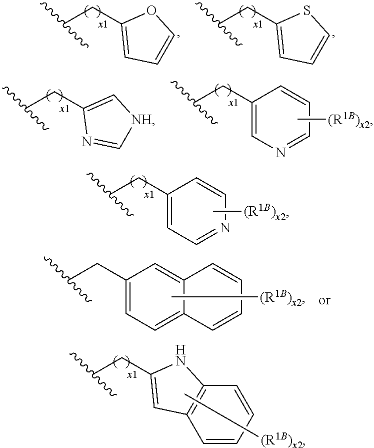

wherein x1 is 1, and x2 is 0. In certain embodiments, R¹ is of the formula:

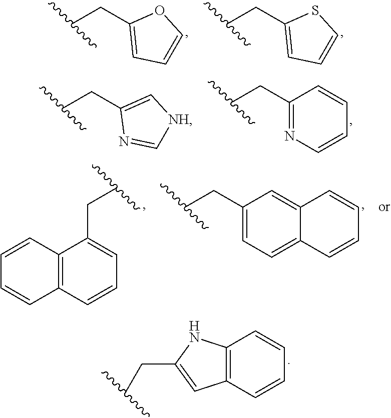

In certain embodiments, R¹ is of the formula:

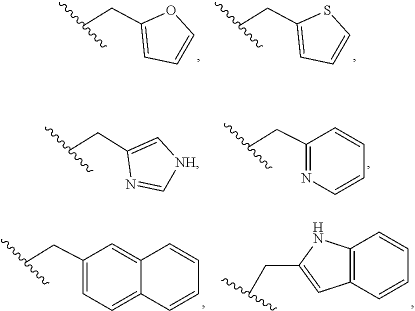

In certain embodiments, R₁ is not of the formula:

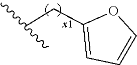

In certain embodiments, R¹ is not of the formula:

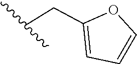 [SMILES: *Cc1ccco1]

In certain embodiments, there are zero instances of R^{1B}. In certain embodiments, x2 is 0. In certain embodiments, there are one or more instances of R^{1B}. In certain embodiments, x2 is 1. In certain embodiments, at least one instance of x2 is 2. In certain embodiments, at least one instance of x2 is 3. In certain embodiments, at least one instance of x2 is 4. In certain embodiments, at least one instance of x2 is 5. In certain embodiments, at least one instance of x2 is 6. In certain embodiments, at least one instance of R^{1B} is halogen (e.g., F, Cl, Br, or I). In certain embodiments, at least one instance of R^{1B} is substituted or unsubstituted acyl (e.g., —C(═O)Me). In certain embodiments, at least one instance of R^{1B} is substituted or unsubstituted alkyl (e.g., substituted or unsubstituted C₁₋₆ alkyl). In certain embodiments, at least one instance of R^{1B} is substituted or unsubstituted methyl. In certain embodiments, at least one instance of R^{1B} is substituted methyl (e.g., —CF₃). In certain embodiments, at least one instance of R^{1B} is unsubstituted methyl. In certain embodiments, at least one instance of R^{1B} is substituted or unsubstituted ethyl. In certain embodiments, at least one instance of R^{1B} is unsubstituted ethyl. In certain embodiments, at least one instance of R^{1B} is substituted or unsubstituted propyl. In certain embodiments, at least one instance of R^{1B} is substituted or unsubstituted butyl (e.g., t-butyl, n-butyl). In certain embodiments, at least one instance of R^{1B} is substituted or unsubstituted alkenyl (e.g., substituted or unsubstituted C₂₋₆ alkenyl). In certain embodiments, at least one instance of R^{1B} is substituted or unsubstituted alkynyl (e.g., substituted or unsubstituted C₂₋₆ alkynyl). In certain embodiments, at least one instance of R^{1B} is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{1B} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{1B} is substituted or unsubstituted aryl (e.g., substituted or unsubstituted, 6- to 10-membered aryl). In certain embodiments, at least one instance R^{1B} benzyl. In certain embodiments, at least one instance of R^{1B} is substituted or unsubstituted phenyl. In certain embodiments, at least one instance of R^{1B} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{1B} is —OR^{c1} (e.g., —OH or —OMe). In certain embodiments, at least one instance of R^{1B} is —O(CH₂)(optionally substituted aryl). In certain embodiments, at least one instance of R^{1B} is —O(CH₂)(phenyl). In certain embodiments, at least one instance of R^{1B} is —O(optionally substituted phenyl). In certain embodiments, at least one instance of R^{1B} is —O(optionally substituted aryl). In certain embodiments, at least one instance of R^{1B} is —NO₂. In certain embodiments, at least one instance of R^{1B} is —N(R^{c2})₂ (e.g., —NMe₂). In certain embodiments, at least one instance of R^{1B} is —SR^{c1} (e.g., —SMe). In certain embodiments, at least one instance of R^{1B} is —SO₂, —CN, or —SCN.

In certain embodiments, at least one instance of R^{1A}, R^{1B}, R^{3A}, R^{3a}, R⁴, R⁵, R^{6A}, R^{6A}, or R^{6B} is —OR^{c1}, —N(R^{c2})₂, or —SR^{c1}, and R^{c1} and R^{c2} as defined herein. In certain embodiments, at least one instance of R^{6A} is OR^{c1}, —NO₂, —N(R^{c2})₂, —SR^{c1}, —SO₂R^{c1}, —CN, —B(OR^{c1})₂, or —SCN; and R^{c1} and R^{c2} are as defined herein. In certain embodiments, at least one instance of R^{6A} is OR^{c1}, —NO₂, —N(R^{c2})₂, —SR^{c1}, —SO₂R^{c1}, —CN, —B(OH)₂, or —SCN; and R^{c1} and R^{c2} are as defined herein.

In certain embodiments, R^{c1} is halogen, hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom, —OH, or —O(optionally substituted alkyl). In certain embodiments, R^{c1} is halogen (e.g., F, Cl, Br, or I), —OH, or —O(optionally substituted alkyl).

In certain embodiments, at least one instance of R^{1A}, R^{1B}, R^{3A}, R^{3a}, R⁴, R⁵, R^{6A}, R^{6A}, or R^{6B} is —OR^{c1}, —N(R^{c2})₂, or —SR^{c1}, and R^{c1} and R^{c2} are as defined herein. In certain embodiments, R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom. In certain embodiments, R^{c1} is hydrogen. In certain embodiments, R^{c1} is substituted or unsubstituted acyl (e.g., —C(═O)Me). In certain embodiments, R^{c1} is substituted or unsubstituted alkyl (e.g., substituted or unsubstituted C₁₋₆ alkyl). In certain embodiments, R^{c1} is substituted or unsubstituted methyl.

In certain embodiments, R^{c1} is substituted or unsubstituted ethyl. In certain embodiments, R^{c1} is substituted or unsubstituted propyl. In certain embodiments, R^{c1} is substituted or unsubstituted alkenyl (e.g., substituted or unsubstituted C₂₋₆ alkenyl). In certain embodiments, R^{c1} is substituted or unsubstituted alkynyl (e.g., substituted or unsubstituted C₂₋₆ alkynyl). In certain embodiments, R^{c1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{c1} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{c1} is substituted or unsubstituted aryl (e.g., substituted or unsubstituted, 6- to 10-membered aryl). In certain embodiments, R^{c1} is benzyl. In certain embodiments, R^{c1} is substituted or unsubstituted phenyl. In certain embodiments, R^{c1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{c1} is an oxygen protecting group when attached to an oxygen atom. In certain embodiments, R^{c1} is a sulfur protecting group when attached to a sulfur atom.

In certain embodiments, at least one instance of R^{c2} is hydrogen. In certain embodiments, at least one instance of R^{c2} is substituted or unsubstituted acyl (e.g., —C(═O)Me). In certain embodiments, at least one R^{c2} is substituted or unsubstituted alkyl (e.g., substituted or unsubstituted C₁₋₆ alkyl). In certain embodiments, at least one instance of R^{c2} is substituted or unsubstituted methyl. In certain embodiments, at least one instance of R^{c2} is substituted or unsubstituted ethyl. In certain embodiments, at least one instance of R^{c2} is substituted or unsubstituted propyl. In certain embodiments, at least one instance of R^{c2} is substituted or unsubstituted alkenyl (e.g., substituted or unsubstituted C₂₋₆ alkenyl). In certain embodiments, at least one instance of R^{c2} is substituted or unsubstituted alkynyl (e.g., substituted or unsubstituted C₂₋₆ alkynyl). In certain embodiments, at least one instance of R_{c2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{c2} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{c2} is substituted or unsubstituted aryl (e.g., substituted or unsubstituted, 6- to 10-membered aryl). In certain embodiments, at least one instance of R^{c2} is benzyl. In certain embodiments, at least one instance of R^{c2} is substituted or unsubstituted phenyl. In certain embodiments, at least one instance of R^{c2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{c2} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R¹ is substituted or unsubstituted alkenyl (e.g., C₂₋₆ alkenyl).

In certain embodiments, R¹ is substituted or unsubstituted C₂₋₆ alkenyl. In certain embodiments, R¹ is C₂₋₆ alkenyl optionally substituted with optionally substituted aryl. In certain embodiments, R¹ is C₂₋₆ alkenyl optionally substituted with optionally substituted phenyl. In certain embodiments, R¹ is substituted or unsubstituted carbocyclyl (e.g., substituted or unsubstituted, 3- to 10-membered, monocyclic carbocyclyl comprising zero, one, or two double bonds in the carbocyclic ring system). In certain embodiments, R¹ is substituted or unsubstituted aryl (e.g., substituted or unsubstituted, 6- to 10-membered aryl).

In certain embodiments, R¹ is substituted or unsubstituted phenyl. In certain embodiments, R¹ is substituted or unsubstituted benzyl.

In certain embodiments, y is 0. In certain embodiments, y is 1.

In certain embodiments, in Formula (I′) or (I), X is —O—, —C(R^{3A})₂—, —C(R^{3A})═, or —N(R^{3B})—, as valency permits. In certain embodiments, X is —O—. In certain embodiments, X is —C(R^{3A})₂— (e.g., —CH₂—). In certain embodiments, X is —C(R^{3A})═(e.g., —CH═). In certain embodiments, X is —N(R^{3B})— (e.g., —NH—).

In certain embodiments, in Formula (I′) or (I), Y is —C(R^{3A})₂— or —N(R^{3B})—, as valency permits. In certain embodiments, Y is —C(R^{3A})₂— (e.g., —CH₂—). In certain embodiments, Y is —N(R^{3B})— (e.g., —NH—).

In certain embodiments, X is —CH₂—; and Y is —NH₂—. In certain embodiments, X is —NH—; and Y is —NH₂—. In certain embodiments, X is —O—; and Y is —NH₂—. In certain embodiments, X is —CH═; and Y is —NH₂—. In certain embodiments, X is —CH₂—; and Y is —CH₂—. In certain embodiments, in compounds of Formula (I′) or (I), each instance of [symbol] is independently a single or double C—C bond, as valency permits. In certain embodiments, in the moiety

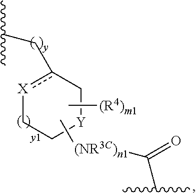

[symbol]

is a single bond. In certain embodiments, in the moiety

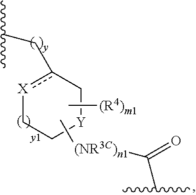

[symbol]

is a double bond.

In certain embodiments, at least one instance of R^{3A} is hydrogen, halogen, substituted or unsubstituted acyl (e.g., —C(═O)Me), substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; wherein R^{c1} and R^{c2} are as defined herein. In certain embodiments, for each of X and Y, both instances of R^{3A} are hydrogen. In certain embodiments, for X, one instance of R^{3A} is hydrogen and the other instance of R^{3A} is substituted or unsubstituted C₁₋₆ alkyl, and for Y, both instances of R^{3A} are hydrogen. In certain embodiments, X or Y is —C(R^{3A})₂—, and two instances of R^{3A} are joined together with the intervening atoms to form an optionally substituted heterocyclyl or heteroaryl ring. In certain embodiments, at least one instance of R^{3A} is —OR^{c1} (e.g., —OMe), —NO₂, —N(R^{c2})₂ (e.g., —NH₂), —SR^{c1} (e.g., —SH), —CN, or —SCN.

In certain embodiments, R^{3B} is hydrogen. In certain embodiments, R^{3B} is substituted or unsubstituted acyl (e.g., —C(═O)Me). In certain embodiments, R^{3B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, in the moiety

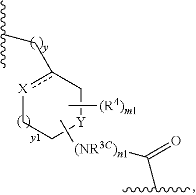

[symbol]

is a single C—C bond, as valency permits. In certain embodiments, in the moiety

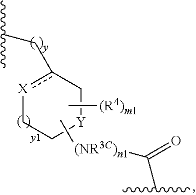

X is —CH═; and [symbol] is a double C—C bond, as valency permits.

In certain embodiments, there are zero instances of R^{3C}. In certain embodiments, n1 is 0. In certain embodiments, there is one instance of R^{3C}. In certain embodiments, n1 is 1. In certain embodiments, at least one instance of R^{3C} is hydrogen. In certain embodiments, at least one instance of R^{3C} is substituted or unsubstituted acyl (e.g., —C(═O)Me). In certain embodiments, at least one instance of R^{3C} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, there are zero instances of R⁴. In certain embodiments, ml is 0. In certain embodiments, there are one or more instances of R⁴. In certain embodiments, m1 is 1. In certain embodiments, at least one instance of m1 is 2. In certain embodiments, at least one instance of m1 is 3. In certain embodiments, at least one instance of m1 is 4. In certain embodiments, at least one instance of m1 is 5. In certain embodiments, at least one instance of m1 is 6.

In certain embodiments, at least one instance of R⁴ is halogen (e.g., F, Cl, Br, or I). In certain embodiments, at least one instance of R⁴ is substituted or unsubstituted acyl (e.g., —C(═O)Me). In certain embodiments, at least one instance of R⁴ is substituted or unsubstituted alkyl (e.g., substituted or unsubstituted C₁₋₆ alkyl). In certain embodiments, at least one instance of R⁴ is substituted or unsubstituted methyl. In certain embodiments, at least one instance of R⁴ is substituted or unsubstituted ethyl. In certain embodiments, at least one instance of R⁴ is substituted or unsubstituted propyl. In certain embodiments, at least one instance of R⁴ is substituted or unsubstituted alkenyl (e.g., substituted or unsubstituted C₂₋₆ alkenyl). In certain embodiments, at least one instance of R⁴ is substituted or unsubstituted alkynyl (e.g., substituted or unsubstituted C₂₋₆ alkynyl). In certain embodiments, at least one instance of R⁴ is substituted or unsubstituted carbocyclyl (e.g., substituted or unsubstituted, 3 to 7-membered, monocyclic carbocyclyl comprising zero, one, or two double bonds in the carbocyclic ring system). In certain embodiments, at least one instance of R⁴ is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R⁴ is substituted or unsubstituted aryl (e.g., substituted or unsubstituted, 6- to 10-membered aryl). In certain embodiments, at least one instance of R⁴ is optionally substituted benzyl. In certain embodiments, at least one instance of R⁴ is benzyl. In certain embodiments, at least one instance of R⁴ is substituted or unsubstituted phenyl. In certain embodiments, at least one instance of R⁴ is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R⁴ is —OR^{c1} (e.g., —OH or —OMe). In certain embodiments, at least one instance of R⁴ is —NO₂. In certain embodiments, at least one instance of R⁴ is —N(R^{c2})₂ (e.g., —NMe₂). In certain embodiments, at least one instance of R⁴ is —SR^{c1} (e.g., —SMe). In certain embodiments, at least one instance of R⁴ is —CN. In certain embodiments, at least one instance of R⁴ is —SCN.

In certain embodiments, the moiety

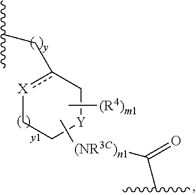

is of formula:

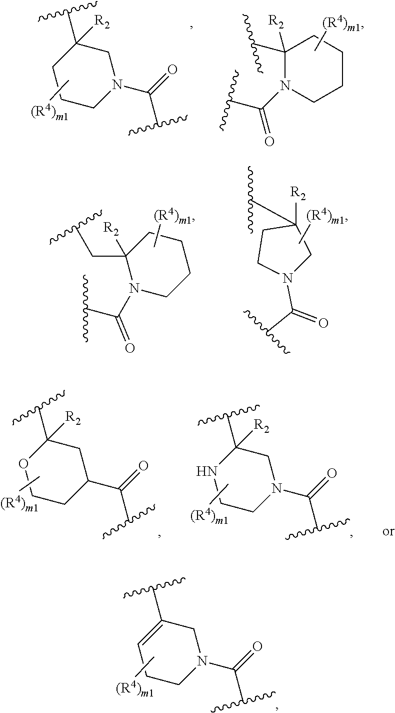

wherein R² and R⁴ are as described herein. In certain embodiments, the moiety

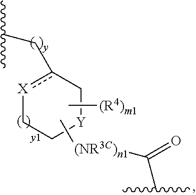

is of formula:

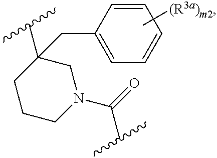

and each instance of R^{3a} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN.

In certain embodiments, the moiety

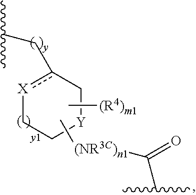

is of formula:

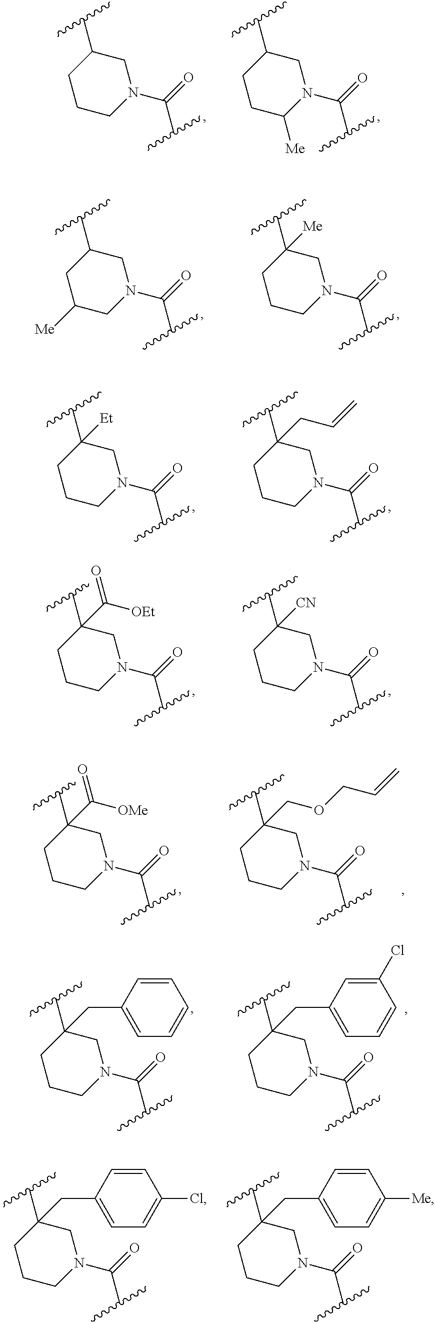

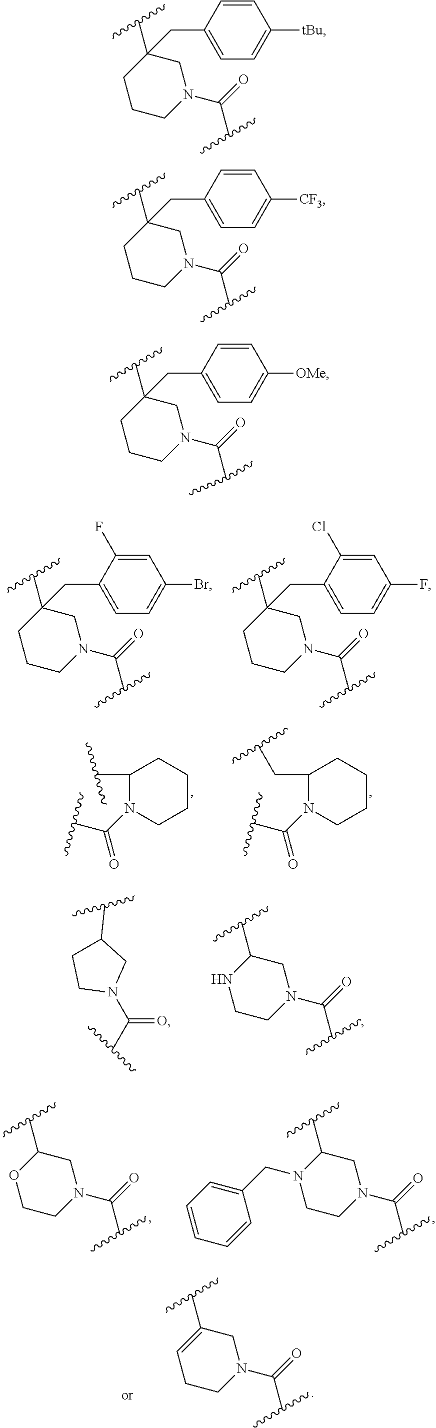

In certain embodiments, the moiety

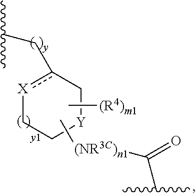

is of formula:

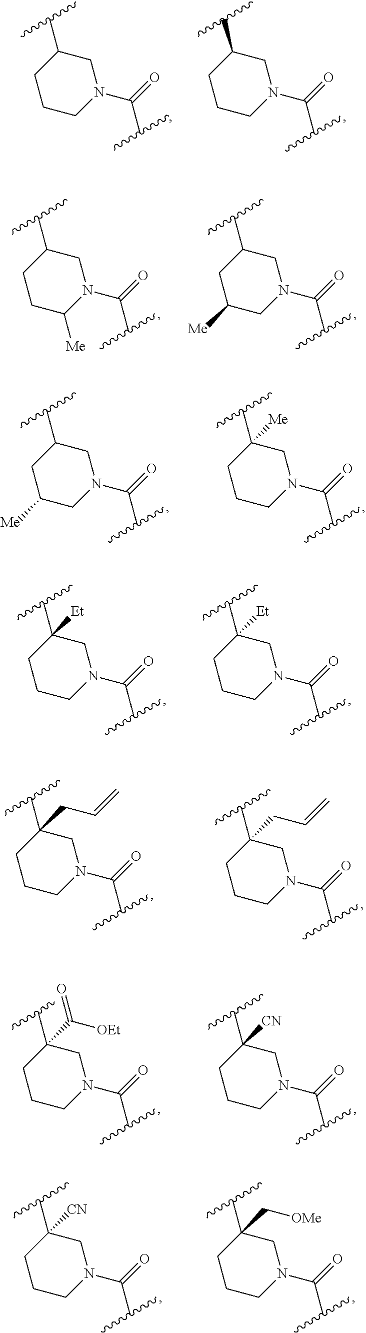

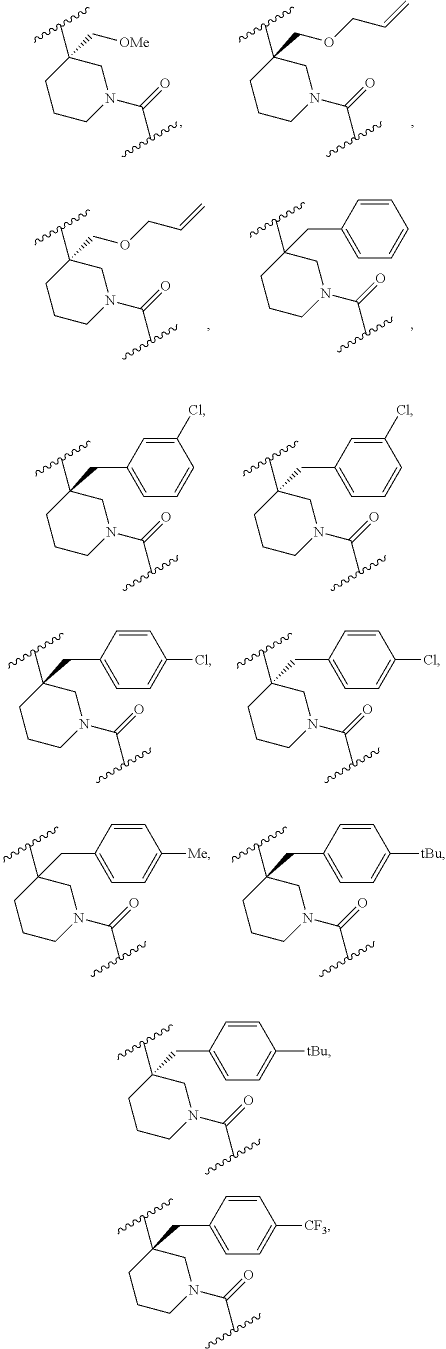

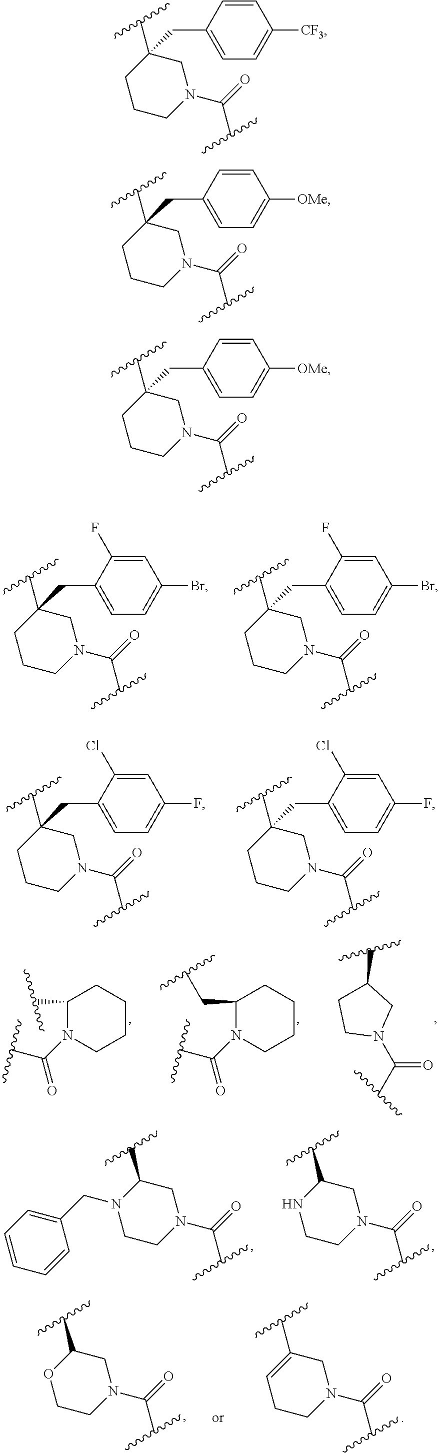

In certain embodiments, the moiety

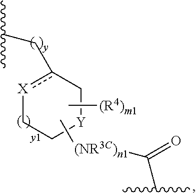

is of formula:

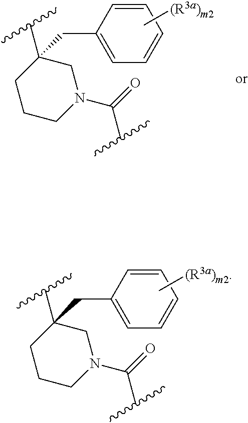

In certain embodiments, the moiety

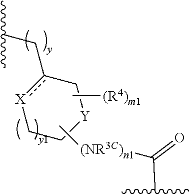

is of formula:

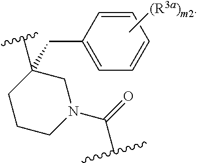

In certain embodiments, the moiety

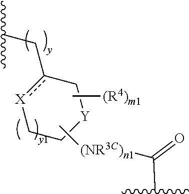

is of formula:

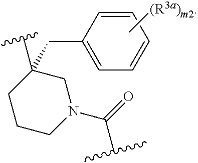

In certain embodiments, the moiety

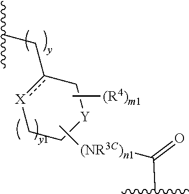

is of formula:

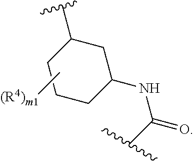

In certain embodiments, the moiety

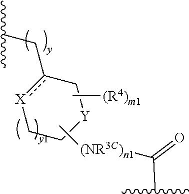

is of formula:

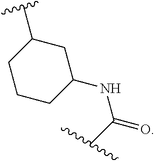 [SMILES: *C(=O)NC1CCCC(*)C1]

In certain embodiments, the moiety

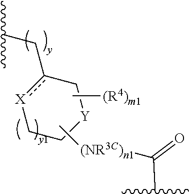

is of formula:

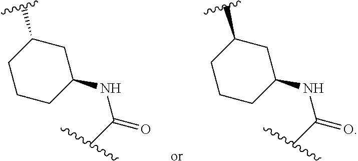 [SMILES: *C(=O)N[C@H]1CCC[C@H](*)C1 ; *C(=O)N[C@H]1CCC[C@@H](*)C1]

In certain embodiments, the compound of Formula (I′) or (I) is of formula:

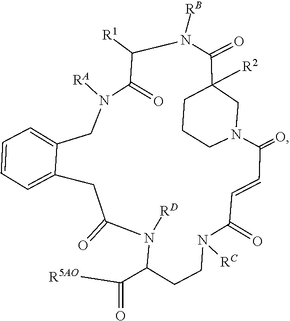

or a pharmaceutically acceptable salt, solvate, hydrate, stereoisomer, polymorph, tautomer, isotopically enriched form, or prodrug thereof, wherein R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN; and R^{5A} is hydrogen or substituted or unsubstituted alkyl; and substituents R¹, R^{A}, R^{B}, R^{C}, and R^{D} are defined as described herein.

In certain embodiments, the compound of Formula (I′) or (I) is of formula:

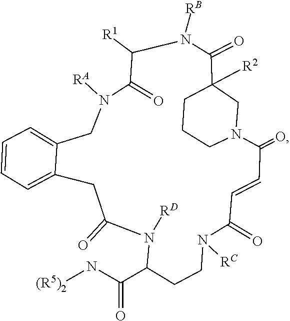

or a pharmaceutically acceptable salt thereof, wherein: R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN. In certain embodiments, R² is hydrogen. In certain embodiments, R² is halogen (e.g., F, Cl, Br, or I). In certain embodiments, R² is substituted or unsubstituted acyl (e.g., —C(═O)Me). In certain embodiments, R² is substituted or unsubstituted alkyl (e.g., substituted or unsubstituted C₁₋₆ alkyl). In certain embodiments, R² is substituted or unsubstituted C₁₋₆ alkyl. In certain embodiments, R² is substituted or unsubstituted methyl. In certain embodiments, R² is methyl optionally substituted with —OR^{c1}, wherein R^{c1} is hydrogen, substituted or unsubstituted acyl, substituted or unsubstituted alkyl, substituted or unsubstituted alkenyl, substituted or unsubstituted alkynyl, or oxygen protecting group. In certain embodiments, R² is methyl optionally substituted with —OH, —O(substituted or unsubstituted C₁₋₆ alkyl), or —O(substituted or unsubstituted C₂₋₆ alkenyl). In certain embodiments, R² is substituted or unsubstituted ethyl. In certain embodiments, R² is substituted or unsubstituted propyl. In certain embodiments, R² is substituted or unsubstituted alkenyl (e.g., substituted or unsubstituted C₂₋₆ alkenyl). In certain embodiments, R² is

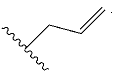 [SMILES: *CC=C]

In certain embodiments, R² is substituted or unsubstituted alkynyl (e.g., substituted or unsubstituted C₂₋₆ alkynyl). In certain embodiments, R² is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R² is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R² is substituted or unsubstituted aryl (e.g., substituted or unsubstituted, 6- to 10-membered aryl). In certain embodiments, R² is benzyl. In certain embodiments, R² is substituted or unsubstituted benzyl. In certain embodiments, R² is substituted or unsubstituted phenyl. In certain embodiments, R² is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R² is —OR^{c1} (e.g., —OH or —OMe). In certain embodiments, R² is —N(R^{c2})₂ (e.g., —NMe₂). In certain embodiments, R² is —SR^{c1} (e.g., —SMe). In certain embodiments, R² is —CN.

In certain embodiments, the compound of Formula (I′) is of formula:

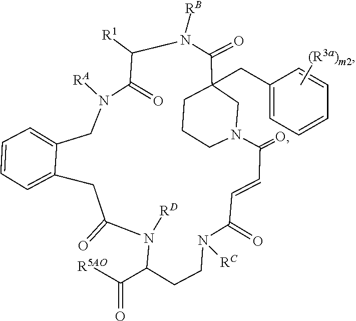

or a pharmaceutically acceptable salt thereof, wherein R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN; and R^{5A} is hydrogen or substituted or unsubstituted alkyl; and the remaining substituents R¹, R^{3a}, R^{A}, R^{B}, R^{C}, R^{D}, and m2 are defined as described herein.

In certain embodiments, the compound of Formula (I) or (I′) is of formula:

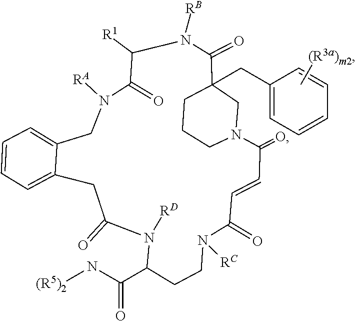

or a pharmaceutically acceptable salt, solvate, hydrate, stereoisomer, polymorph, tautomer, isotopically enriched form, or prodrug thereof, wherein: each instance of R^{3a} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; and m2 is 0, 1, 2, 3, 4, or 5. In certain embodiments, the compound of Formula (I′) or (I) is of formula:

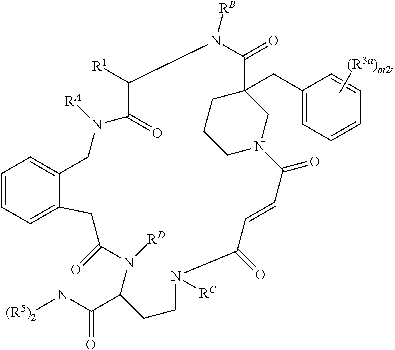

or a pharmaceutically acceptable salt thereof, wherein the definitions of R^{3a} and m2 are as provided herein.

In certain embodiments, the compound of Formula (I′) or (I) is of formula:

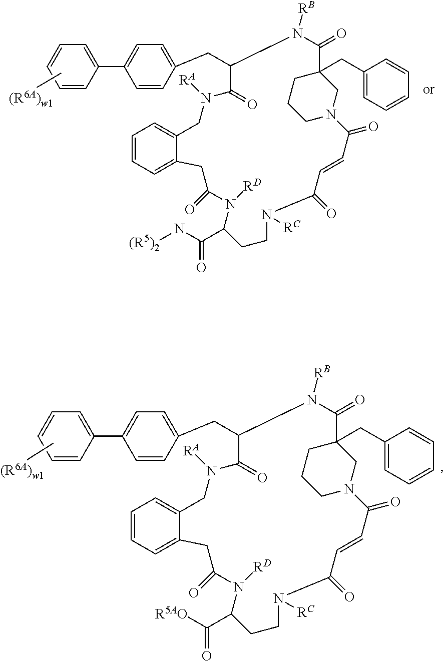

or a pharmaceutically acceptable salt, solvate, hydrate, stereoisomer, polymorph, tautomer, isotopically enriched form, or prodrug thereof, wherein: each instance of R^{6A} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, —B(OR^{6c1})₂, or —SCN; and w1 is 0, 1, 2, 3, 4, 5, or 6.

In certain embodiments, the compound of Formula (I′) or (I) is of formula:

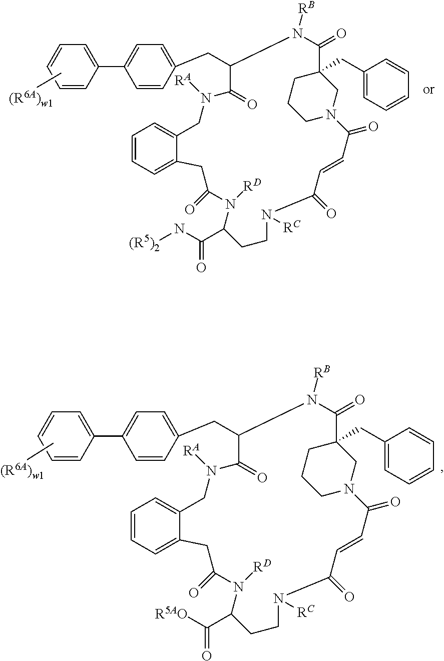

or a pharmaceutically acceptable salt, solvate, hydrate, stereoisomer, polymorph, tautomer, isotopically enriched form, or prodrug thereof. In certain embodiments, the compound of Formula (I′) or (I) is of formula:

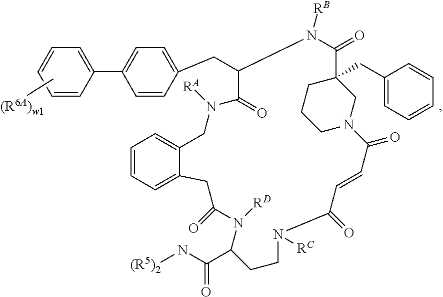

or a pharmaceutically acceptable salt, solvate, hydrate, stereoisomer, polymorph, tautomer, isotopically enriched form, or prodrug thereof. In certain embodiments, the compound of Formula (I′) or (I) is of formula:

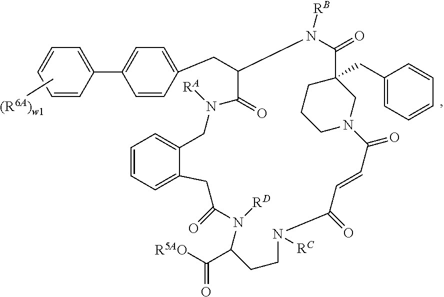

or a pharmaceutically acceptable salt, solvate, hydrate, stereoisomer, polymorph, tautomer, isotopically enriched form, or prodrug thereof.

In certain embodiments, the compound of Formula (I) or (I′) is of formula:

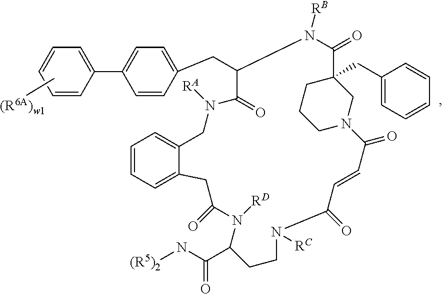

or a pharmaceutically acceptable salt, solvate, hydrate, stereoisomer, polymorph, tautomer, isotopically enriched form, or prodrug thereof.

In certain embodiments, in the moiety

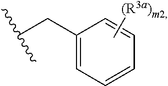

there are zero instances of R^{3a}. In certain embodiments, there are zero instances of R^{3a}. In certain embodiments, m2 is 0. In certain embodiments, there are one or more instances of R^{3a}. In certain embodiments, m2 is 1. In certain embodiments, at least one instance of m2 is 2. In certain embodiments, at least one instance of m2 is 3. In certain embodiments, at least one instance of m2 is 4. In certain embodiments, at least one instance of m2 is 5. In certain embodiments, at least one instance of m2 is 6. In certain embodiments, at least one instance of R^{3a} is halogen (e.g., F, Cl, Br, or I). In certain embodiments, m2 is 2 and both instances of R^{3a} are halogen (e.g., F, Cl, Br, or I). In certain embodiments, at least one instance of R^{3a} is substituted or unsubstituted acyl (e.g., —C(═O)Me). In certain embodiments, at least one instance of R^{3a} is substituted or unsubstituted alkyl (e.g., substituted or unsubstituted C₁₋₆ alkyl). In certain embodiments, at least one instance of R^{3a} is substituted or unsubstituted methyl. In certain embodiments, at least one instance of R^{3a} is methyl optionally substituted with halogen. In certain embodiments, at least one instance of R^{3a} is —CF₃. In certain embodiments, at least one instance of R^{3a} is substituted or unsubstituted ethyl. In certain embodiments, at least one instance of R^{3a} is substituted or unsubstituted propyl. In certain embodiments, at least one instance of R^{3a} is substituted or unsubstituted butyl (e.g., substituted or unsubstituted n-butyl or substituted or unsubstituted t-butyl). In certain embodiments, at least one instance of R^{3a} is substituted or unsubstituted t-butyl. In certain embodiments, at least one instance of R^{3a} is unsubstituted t-butyl. In certain embodiments, at least one instance of R^{3a} is substituted or unsubstituted alkenyl (e.g., substituted or unsubstituted C₂₋₆ alkenyl). In certain embodiments, at least one instance of R^{3a} is substituted or unsubstituted alkynyl (e.g., substituted or unsubstituted C₂₋₆ alkynyl). In certain embodiments, at least one instance of R^{3a} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{3a} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{3a} is substituted or unsubstituted aryl (e.g., substituted or unsubstituted, 6- to 10-membered aryl). In certain embodiments, at least one instance of R^{3a} is benzyl. In certain embodiments, at least one instance of R^{3a} is substituted or unsubstituted phenyl. In certain embodiments, at least one instance of R^{3a} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{3a} is —OR^{c1} (e.g., —OH or —OMe). In certain embodiments, at least one instance of R^{3a} is —O(optionally substituted C₁₋₆ alkyl). In certain embodiments, at least one instance of R^{3a} is —OMe. In certain embodiments, at least one instance of R^{3a} is —OEt. In certain embodiments, at least one instance of R^{3a} is —O(optionally substituted C₂₋₆ alkenyl). In certain embodiments, at least one instance of R^{3a} is

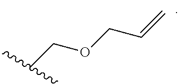 [SMILES: *COCC=C]

In certain embodiments, at least one instance of R^{3a} is —NO₂. In certain embodiments, at least one instance of R^{3a} is —N(R^{c2})₂ (e.g., —NMe₂). In certain embodiments, at least one instance of R^{3a} is —SR^{c1} (e.g., —SMe). In certain embodiments, at least one instance of R^{3a} is —CN. In certain embodiments, at least one instance of R^{3a} is —SCN.

In certain embodiments, y1 is 0. In certain embodiments, y1 is 1.

Compounds of Formula (I′) or (I) include substituent A that is —OR^{5A} or —N(R⁵)₂, wherein R^{5A} and R⁵ are as defined herein. In certain embodiments, A is —OR^{5A}, wherein R^{5A} is hydrogen, substituted or unsubstituted acyl, substituted or unsubstituted alkyl, substituted or unsubstituted alkenyl, substituted or unsubstituted alkynyl, or an oxygen protecting group. In certain embodiments, R^{5A} is hydrogen. In certain embodiments, R^{5A} is substituted or unsubstituted acyl (e.g., —C(═O)Me). In certain embodiments, R^{5A} is substituted or unsubstituted alkyl (e.g., substituted or unsubstituted C₁₋₆ alkyl). In certain embodiments, R^{5A} is optionally substituted C₁₋₆ alkyl. In certain embodiments, R^{5A} is substituted or unsubstituted methyl. In certain embodiments, R^{5A} is methyl. In certain embodiments, R^{5A} is an oxygen protecting group (e.g., methyl, methoxylmethyl (MOM), trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), methanesulfonate (mesylate), benzylsulfonate, and tosylate (Ts)).

In certain embodiments, A is —N(R⁵)₂, wherein R⁵ is as defined herein. In certain embodiments, A is —N(R⁵)₂, wherein each instance of R⁵ is independently hydrogen, substituted or unsubstituted acyl, substituted or unsubstituted alkyl, substituted or unsubstituted alkenyl, substituted or unsubstituted alkynyl, or a nitrogen protecting group. In certain embodiments, at least one instance of R⁵ is hydrogen. In certain embodiments, both instances of R⁵ are hydrogen. In certain embodiments, at least one instance of R⁵ is substituted or unsubstituted acyl (e.g., —C(═O)Me). In certain embodiments, at least one instance of R⁵ is substituted or unsubstituted alkyl (e.g., substituted or unsubstituted C₁₋₆ alkyl). In certain embodiments, at least one instance of R⁵ is C₁₋₆ alkyl optionally substituted with halogen, —OR^{c1}, or —N(R^{c2})₂; and R^{c1} is hydrogen or C₁₋₆ alkyl optionally substituted with —N(R^{c2})₂ and each instance of R^{c2} is independently hydrogen, optionally substituted acyl, optionally substituted alkyl, optionally substituted alkenyl, optionally substituted alkynyl, or a nitrogen protecting group. In certain embodiments, at least one instance of R⁵ is C₁₋₆ alkyl optionally substituted with —OR^{c1}, and R^{c1} is C₁₋₆ alkyl optionally substituted with —N(R^{c2})₂, and each instance of R^{c2} is independently hydrogen, optionally substituted acyl, optionally substituted alkyl, optionally substituted alkenyl, optionally substituted alkynyl, or a nitrogen protecting group. In certain embodiments, at least one instance of R⁵ is substituted or unsubstituted methyl. In certain embodiments, at least one instance of R⁵ is unsubstituted methyl. In certain embodiments, at least one instance of R⁵ is substituted or unsubstituted ethyl. In certain embodiments, at least one instance of R⁵ is ethyl optionally substituted with —OR^{c1}, and R^{c1} is substituted or unsubstituted C₁₋₆ alkyl. In certain embodiments, at least one instance of R⁵ is ethyl optionally substituted with —OR^{c1}, and R^{c1} is C₁₋₆ alkyl optionally substituted with —NH₂. In certain embodiments, at least one instance of R⁵ is of formula

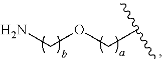

b, wherein a is 0, 1, 2, 3, 4, 5, or 6; and b is 0, 1, 2, 3, 4, 5, or 6. In certain embodiments, at least one instance of R⁵ is of formula:

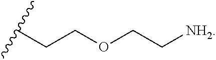 [SMILES: *CCOCCN]

In certain embodiments, at least one instance of R⁵ is methyl or of formula

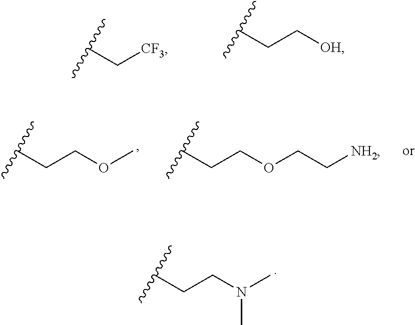

In certain embodiments, at least one instance of R⁵ is substituted or unsubstituted propyl. In certain embodiments, at least one instance of R⁵ is substituted or unsubstituted alkenyl (e.g., substituted or unsubstituted C₂₋₆ alkenyl). In certain embodiments, at least one instance of R⁵ is substituted or unsubstituted alkynyl (e.g., substituted or unsubstituted C₂₋₆ alkynyl). In certain embodiments, at least one instance of R⁵ is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, one instance of R⁵ is hydrogen; and the other instance of R⁵ is substituted or unsubstituted C₁₋₆ alkyl. In certain embodiments, one instance of R⁵ is hydrogen; and the other instance of R⁵ is of formula:

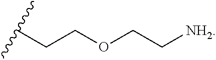 [SMILES: *CCOCCN]

In certain embodiments, one instance of R⁵ is hydrogen; and the other instance of R⁵ is of formula:

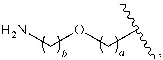

wherein a is 0, 1, 2, 3, 4, 5, or 6; and b is 0, 1, 2, 3, 4, 5, or 6. In certain embodiments, one instance of R⁵ is hydrogen; and the other instance of R⁵ is of formula:

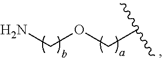

wherein a is 0, 1, 2, or 3; and b is 0, 1, 2, or 3.
In certain embodiments, the moiety

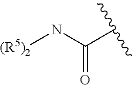

is

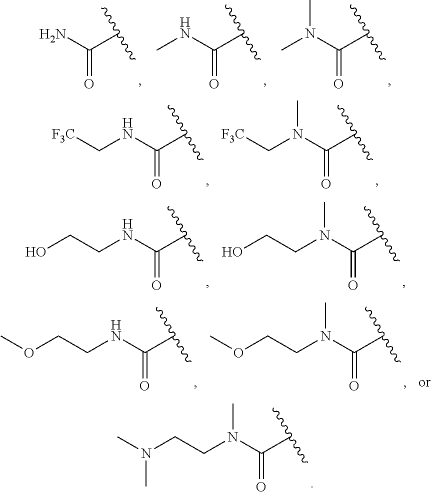

In certain embodiments, the compound of Formula (I′) or (I) is of formula:

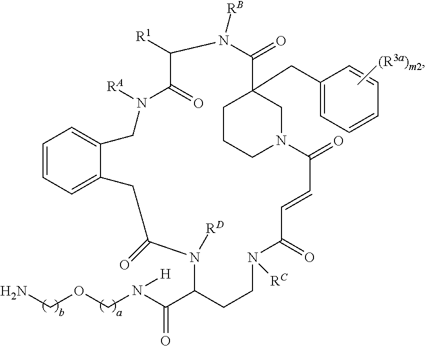

or a pharmaceutically acceptable salt thereof, wherein a is 0, 1, 2, 3, 4, 5, or 6; and b is 0, 1, 2, 3, 4, 5, or 6.

Each of R^{A}, R^{B}, R^{C}, R^{D}, and R^{W} is independently hydrogen, substituted or unsubstituted alkyl, substituted or unsubstituted acyl, or a nitrogen protecting group. In certain embodiments, R^{A} is hydrogen. In certain embodiments, R^{A} is substituted or unsubstituted acyl (e.g., —C(═O)Me). In certain embodiments, R^{A} is substituted or unsubstituted alkyl (e.g., substituted or unsubstituted C₁₋₆ alkyl). In certain embodiments, R^{A} is substituted or unsubstituted C₁₋₆ alkyl. In certain embodiments, R^{A} is substituted or unsubstituted methyl. In certain embodiments, R^{A} is unsubstituted methyl. In certain embodiments, R^{A} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{B} is hydrogen. In certain embodiments, R^{B} is substituted or unsubstituted acyl (e.g., —C(═O)Me). In certain embodiments, R^{B} is substituted or unsubstituted alkyl (e.g., substituted or unsubstituted C₁₋₆ alkyl). In certain embodiments, R^{B} is substituted or unsubstituted C₁₋₆ alkyl. In certain embodiments, R^{B} is substituted or unsubstituted methyl. In certain embodiments, R^{B} is unsubstituted methyl. In certain embodiments, R^{B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{C} is hydrogen. In certain embodiments, R^{C} is substituted or unsubstituted acyl (e.g., —C(═O)Me). In certain embodiments, R^{C} is substituted or unsubstituted alkyl (e.g., substituted or unsubstituted C₁₋₆ alkyl). In certain embodiments, R^{C} is substituted or unsubstituted C₁₋₆ alkyl. In certain embodiments, R^{C} is substituted or unsubstituted methyl. In certain embodiments, R^{C} is substituted or unsubstituted ethyl. In certain embodiments, R^{C} is unsubstituted methyl. In certain embodiments, R^{C} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R^{D} is hydrogen. In certain embodiments, R^{D} is substituted or unsubstituted acyl (e.g., —C(═O)Me). In certain embodiments, R^{D} is substituted or unsubstituted alkyl (e.g., substituted or unsubstituted C₁₋₆ alkyl). In certain embodiments, R^{D} is substituted or unsubstituted C₁₋₆ alkyl. In certain embodiments, R^{D} is substituted or unsubstituted methyl. In certain embodiments, R^{D} is unsubstituted methyl. In certain embodiments, R^{D} is substituted or unsubstituted ethyl. In certain embodiments, R^{D} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, each of R^{A}, R^{B}, R^{C}, and R^{D} is hydrogen. In certain embodiments, one of R^{A}, R^{B}, R^{C}, and R^{D} is substituted or unsubstituted C₁₋₆ alkyl and the rest of R^{A}, R^{B}, R^{C}, and R^{D} are each hydrogen. In certain embodiments, R^{B} is substituted or unsubstituted C₁₋₆ alkyl (e.g., methyl) and the rest of R^{A}, R^{C}, and R^{D} are each hydrogen. In certain embodiments, R^{B} is methyl and the rest of R^{A}, R^{C}, and R^{D} are each hydrogen.

In certain embodiments, W is unsubstituted n-butylene or of formula:

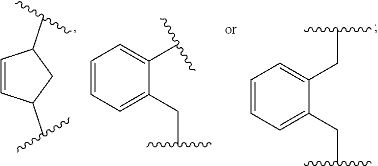

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

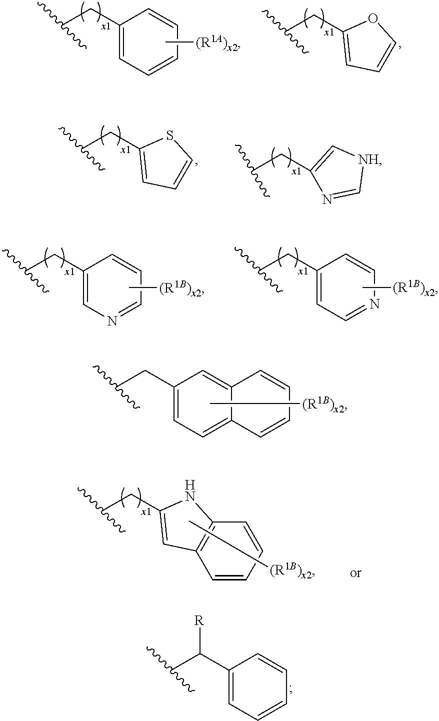

x1 is 0 or 1; the moiety

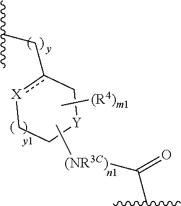

is of formula:

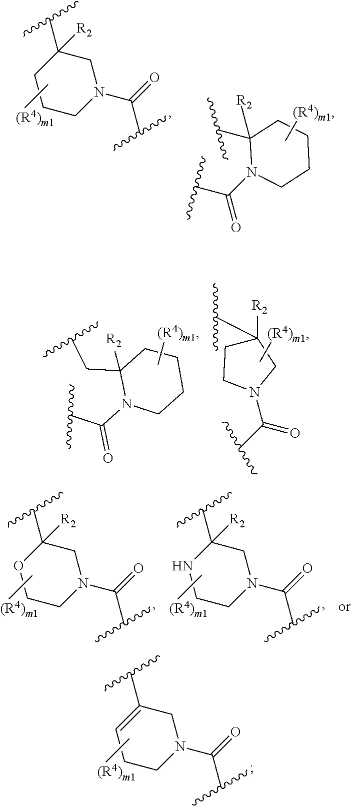

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

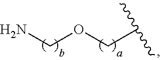

wherein a is 0, 1, 2, or 3; and b is 0, 1, 2, or 3. In certain embodiments, W is unsubstituted n-butylene; x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

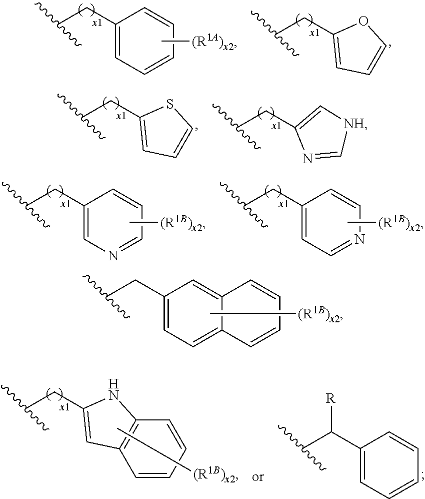

x1 is 0 or 1; the moiety

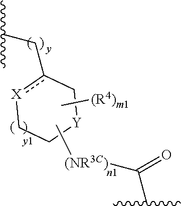

is of formula:

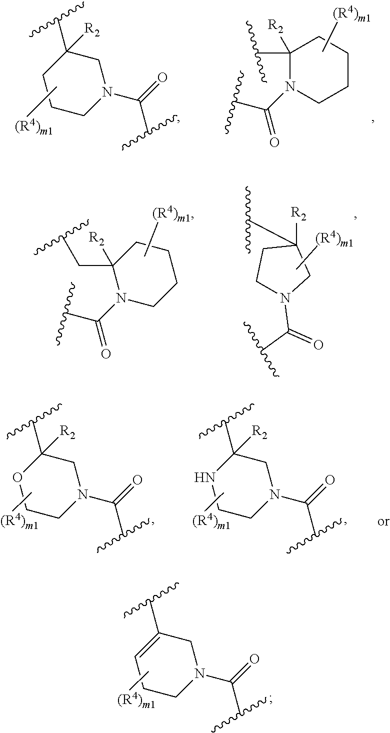

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

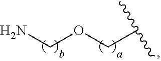

wherein a is 0, 1, 2, or 3; and b is 0, 1, 2, or 3. In certain embodiments, W is of formula:

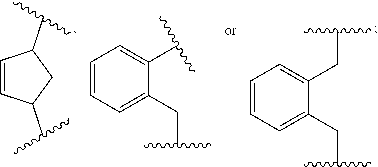

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

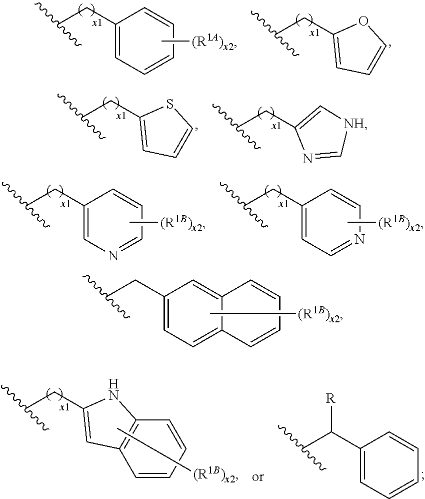

x1 is 0 or 1; the moiety

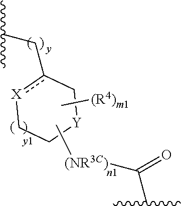

is of formula:

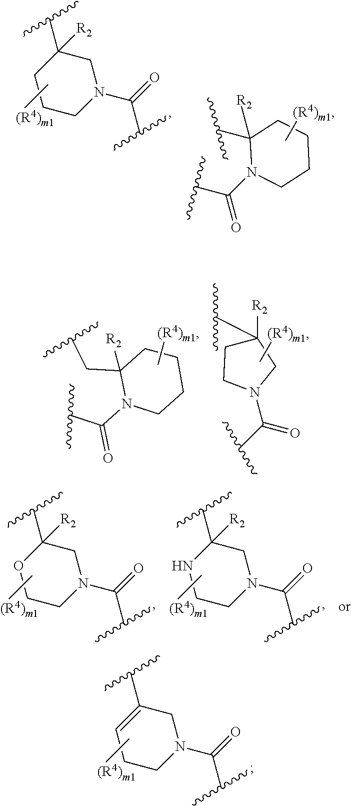

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

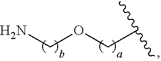

wherein a is 0, 1, 2, or 3; and b is 0, 1, 2, or 3.

In certain embodiments, W is unsubstituted n-butylene or of formula:

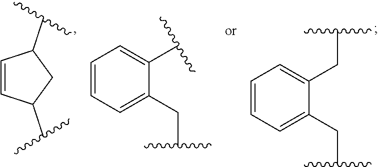

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

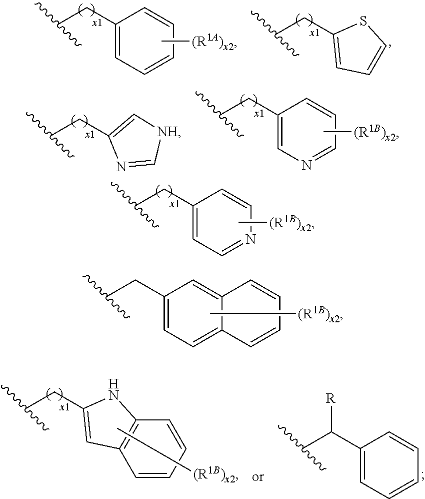

x1 is 0 or 1; the moiety

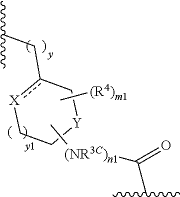

is of formula:

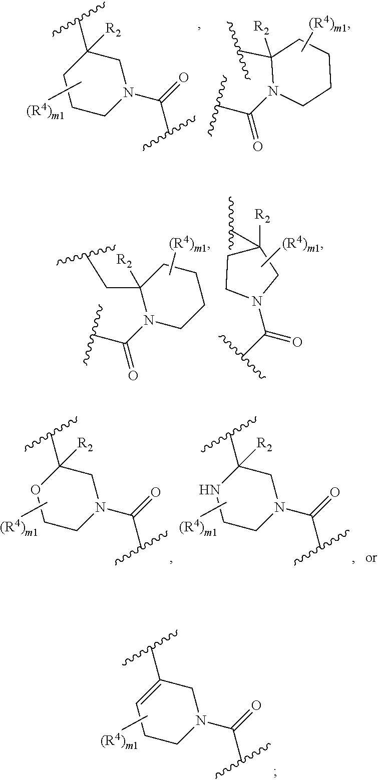

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

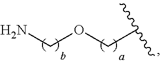

wherein a is 0, 1, 2, or 3; and b is 0, 1, 2, or 3. In certain embodiments, W is unsubstituted n-butylene or of formula:

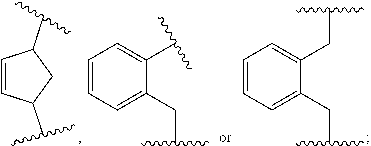

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

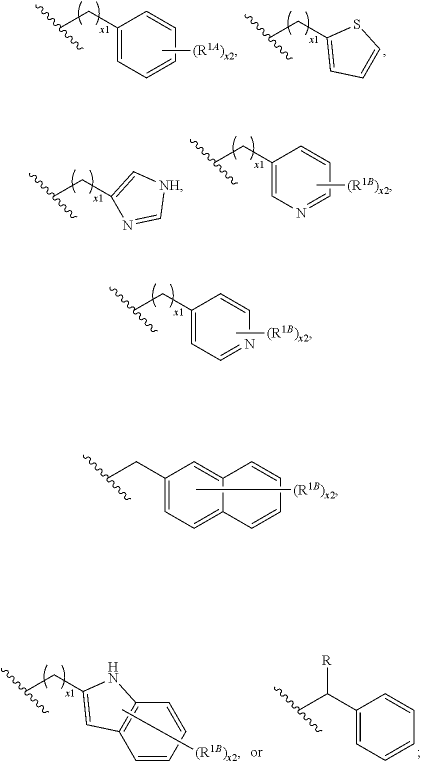

x1 is 0 or 1; the moiety

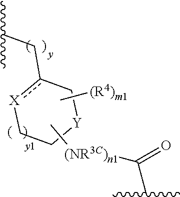

is of formula:

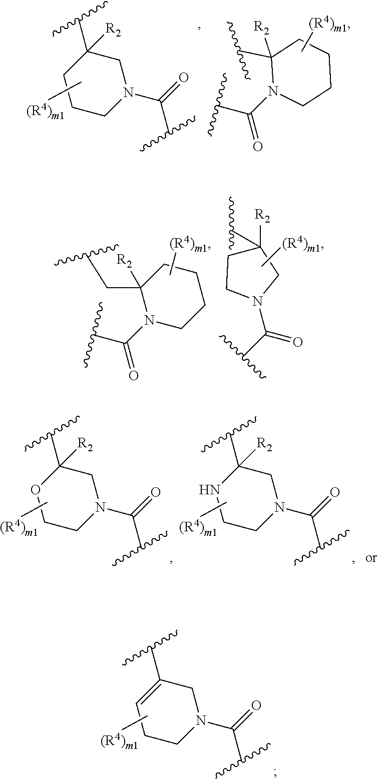

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

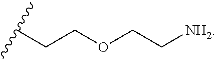 [SMILES: *CCOCCN]

In certain embodiments, W is unsubstituted n-butylene; x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

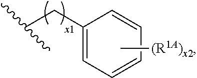

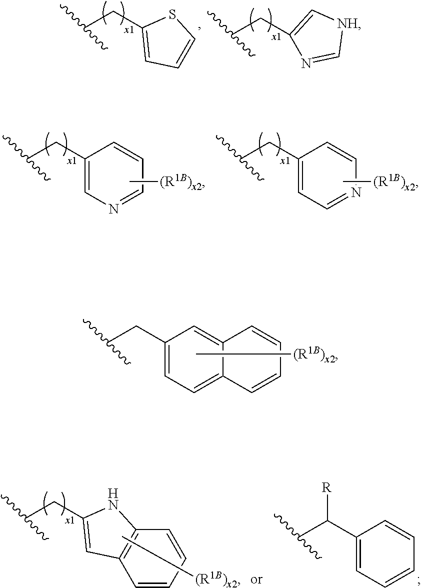

x1 is 0 or 1; the moiety

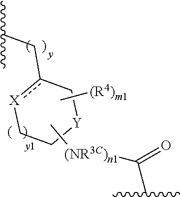

is of formula:

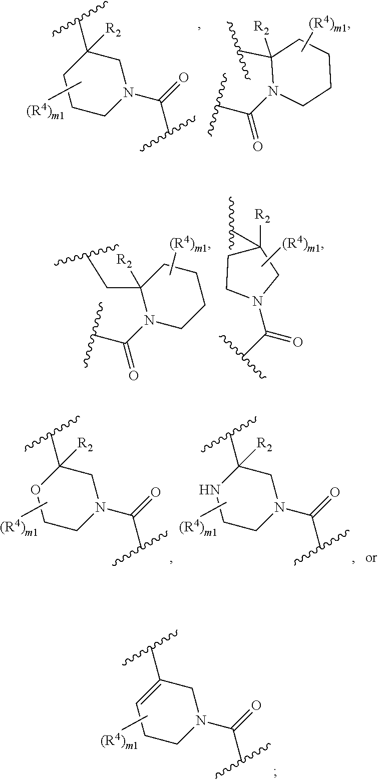

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

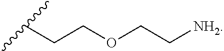 [SMILES: *CCOCCN]

In certain embodiments, W is of formula:

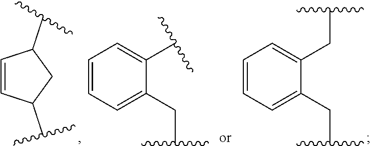

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

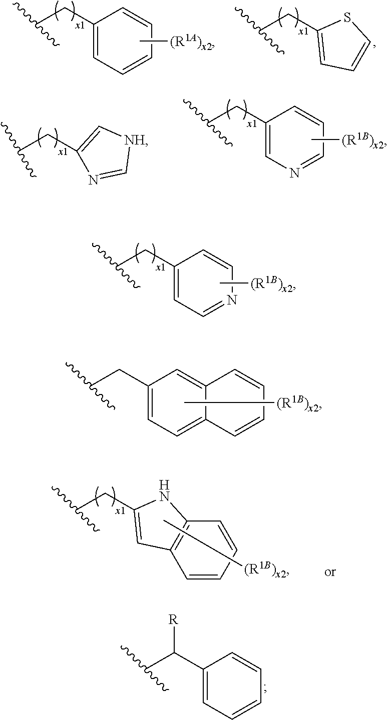

x1 is 0 or 1; the moiety

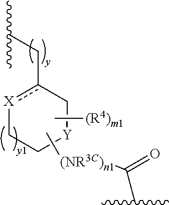

is of formula:

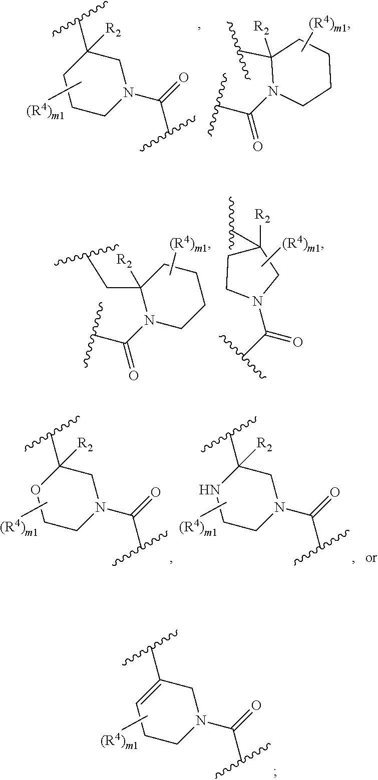

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen and the other instance of R⁵ is of formula

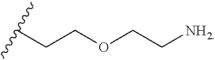 [SMILES: *CCOCCN]

In certain embodiments, W is unsubstituted n-butylene or of formula:

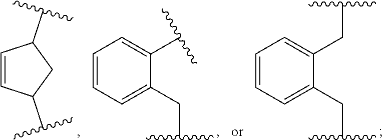

x is 1; R¹ is unsubstituted straight-chain or branched C₁₋₆ alkyl or straight-chain C₁₋₆ alkyl substituted with —OH or unsubstituted C₁₋₆

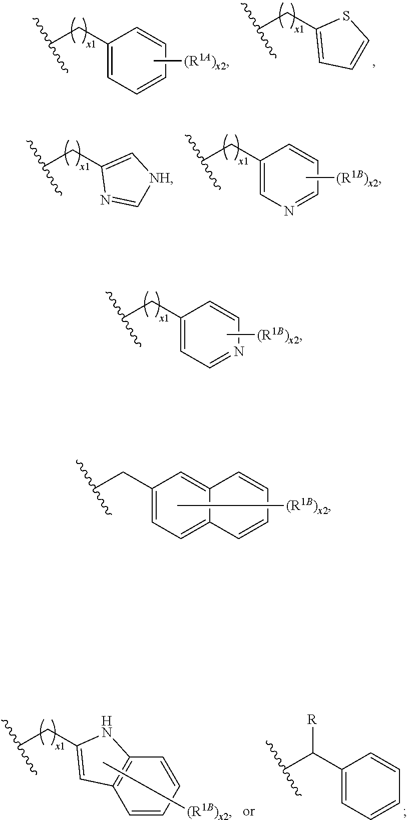

x1 is 0 or 1; the moiety

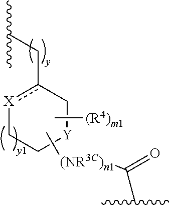

is of formula:

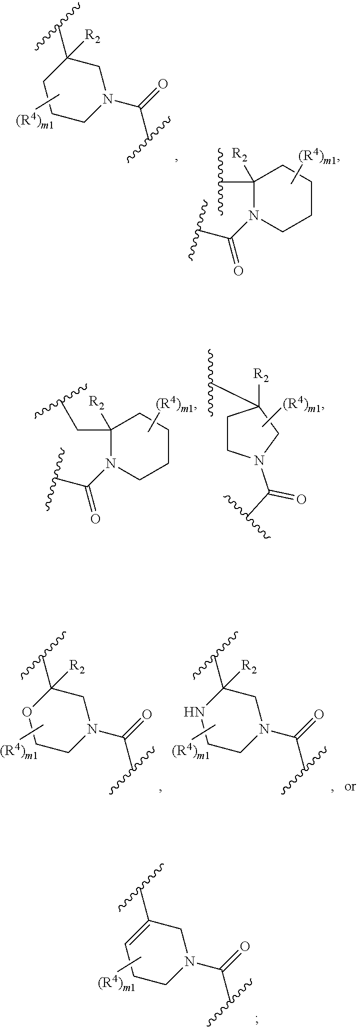

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl; and the rest of R^{A}, R^{B}, R^{C}, and R^{D} are each hydrogen; one instance of R⁵ is hydrogen; and the other instance of R⁵ is of formula

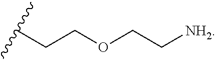 [SMILES: *CCOCCN]

In certain embodiments, W is unsubstituted n-butylene or of formula:

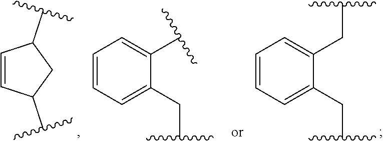

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

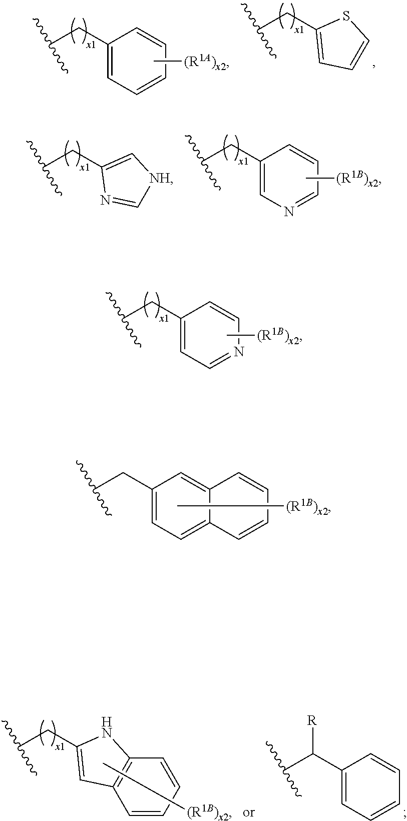

x1 is 0 or 1; the moiety

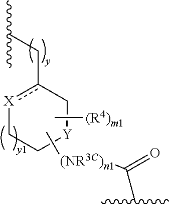

is of formula:

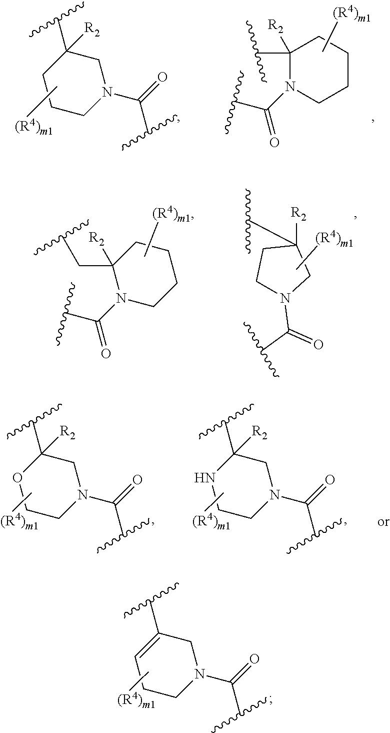

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; each of R^{A}, R^{B}, R^{C}, and R^{D} is hydrogen; one instance of R⁵ is hydrogen; and the other instance of R⁵ is of formula

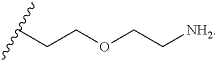 [SMILES: *CCOCCN]

In certain embodiments, W is unsubstituted n-butylene or of formula:

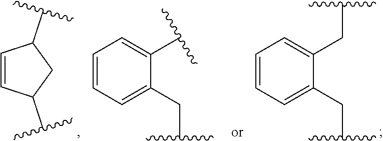

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

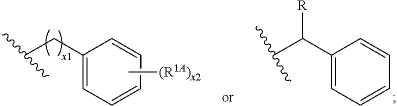

x1 is 0 or 1; the moiety

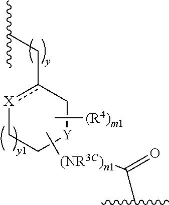

is of formula:

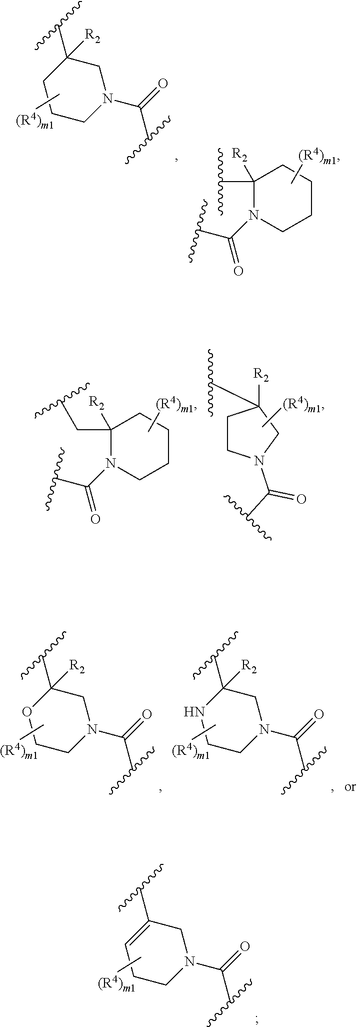

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl and the rest of R^{A}, R^{B}, R^{C}, and R^{D} are each hydrogen; one instance of R⁵ is hydrogen and the other instance of R⁵ is of formula

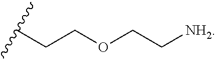 [SMILES: *CCOCCN]

In certain embodiments, W is unsubstituted n-butylene; x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

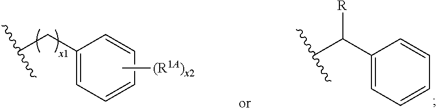

x1 is 0 or 1; the moiety

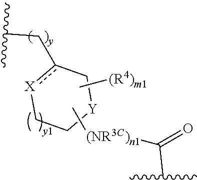

is of formula:

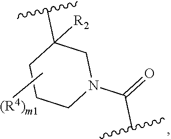

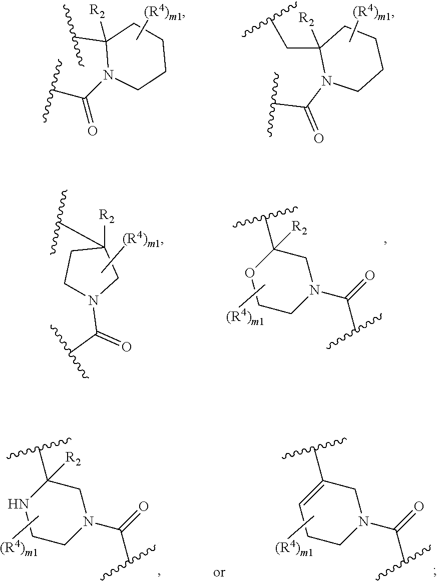

m1 is 0 or 1; R² is hydrogen, substituted or unsubstituted alkenyl, substituted or unsubstituted benzyl, or methyl optionally substituted with —OR^{c1}, or wherein R¹ is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl and the rest of R^{A}, R^{B}, R^{C}, and R^{D} are each hydrogen; one instance of R⁵ is hydrogen and the other instance of R⁵ is of formula

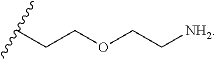 [SMILES: *CCOCCN]

In certain embodiments, W is of formula:

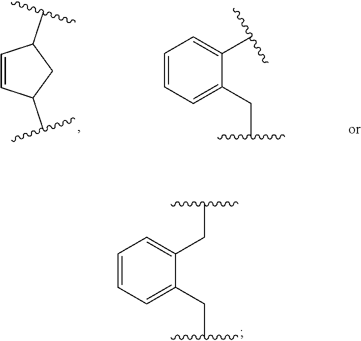 [SMILES: *C1C=CC(*)C1 ; *Cc1ccccc1* ; *Cc1ccccc1C*]

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

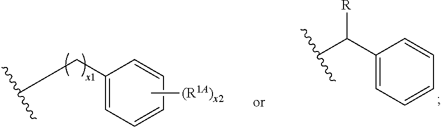

x1 is 0 or 1; the moiety

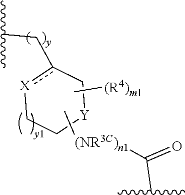

is of formula

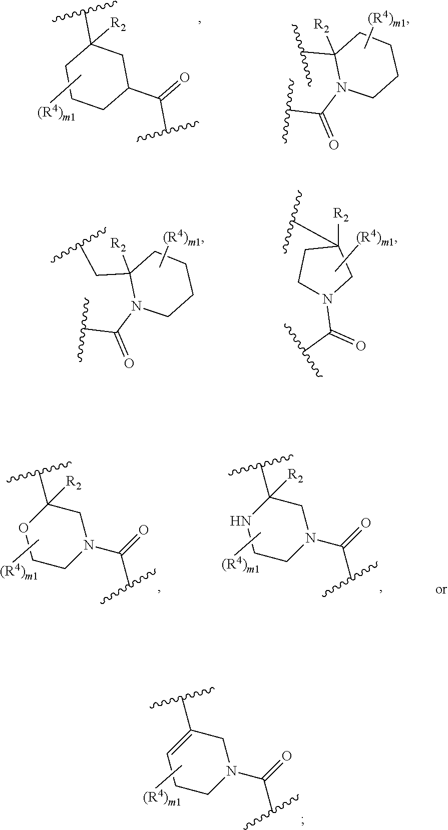

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl and the rest of R^{A}, R^{B}, R^{C}, and R^{D} are each hydrogen; one instance of R⁵ is hydrogen and the other instance of R⁵ is of

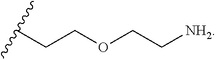 [SMILES: *CCOCCN]

In certain embodiments, W is unsubstituted n-butylene or of formula:

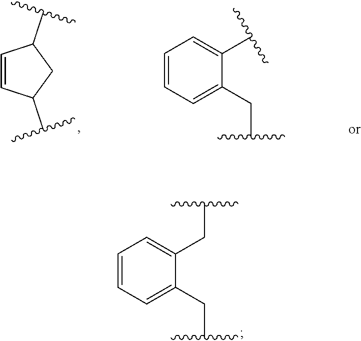 [SMILES: *C1C=CC(*)C1 ; *Cc1ccccc1* ; *Cc1ccccc1C*]

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

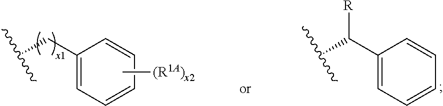

x1 is 0 or 1; the moiety

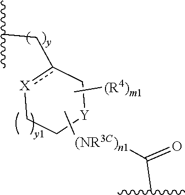

is of formula:

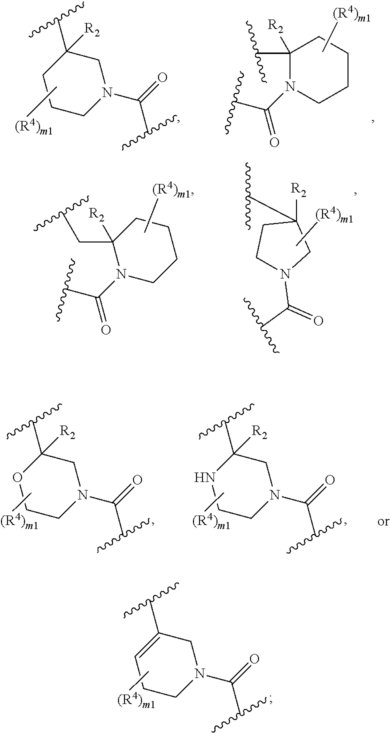

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl and the rest of R^{A}, R^{B}, R^{C}, and R^{D} are each hydrogen; one instance of R⁵ is hydrogen and the other instance of R⁵ is of formula

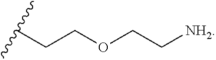 [SMILES: *CCOCCN]

In certain embodiments, W is unsubstituted n-butylene; x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

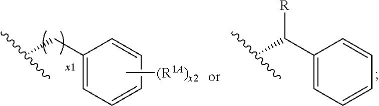

x1 is 0 or 1; the moiety

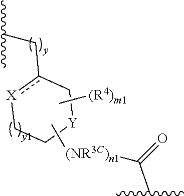

is of formula:

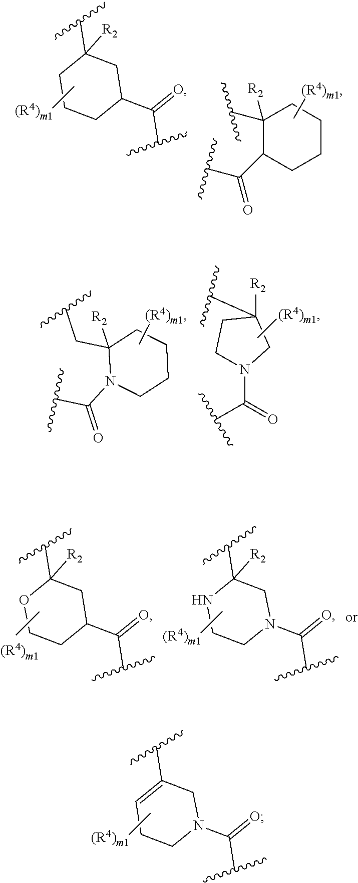

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl; and the rest of R^{A}, R^{B}, R^{C}, and R^{D} are each hydrogen; one instance of R⁵ is hydrogen; and the other instance of R⁵ is of formula

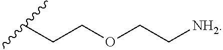 [SMILES: *CCOCCN]

In certain embodiments, W is of formula:

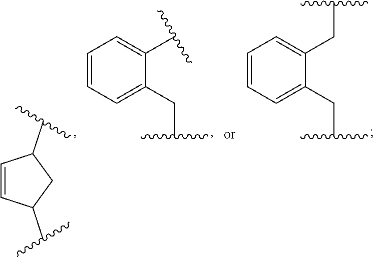 [SMILES: *C1C=CC(*)C1 ; *Cc1ccccc1* ; *Cc1ccccc1C*]

x is 1; R¹ is unsubstituted straight-chain or branched C₁₋₆ alkyl or straight-chain C₁₋₆ alkyl substituted with —OH or —O(unsubstituted C₁₋₆ alkyl), or of formula:

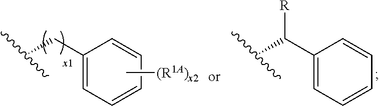

x1 is 0 or 1; the moiety

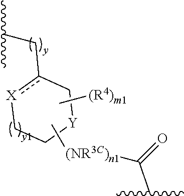

is of formula:

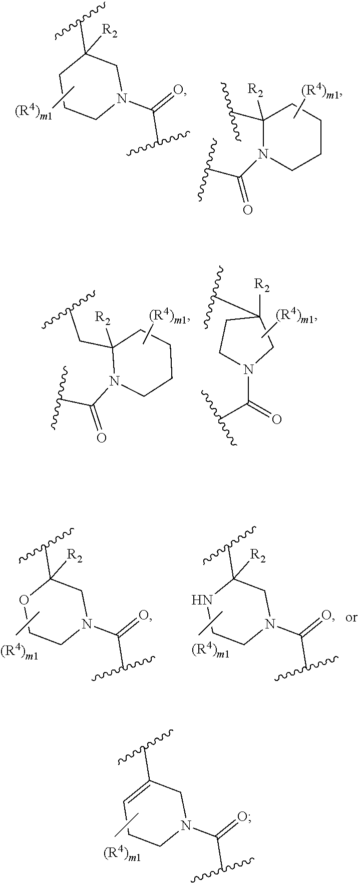

m1 is 0 or 1; R² is hydrogen, substituted or unsubstituted alkenyl, substituted or unsubstituted benzyl, or methyl optionally substituted with —OR^{c1}, or wherein R^{c1} is hydrogen, substituted or unsubstituted acyl, substituted or unsubstituted alkyl, substituted or unsubstituted alkenyl, substituted or unsubstituted alkynyl; R⁴ is substituted or unsubstituted benzyl; one of R^{A}, R^{B}, R^{C}, and R^{D} is substituted or unsubstituted C₁₋₆ alkyl and the rest of R^{A}, R^{B}, R^{C}, and R^{D} are each hydrogen; one instance of R⁵ is hydrogen and the other instance of R⁵ is of formula

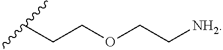 [SMILES: *CCOCCN]

In certain embodiments, the compound of Formula (I′) or (I) is of formula:

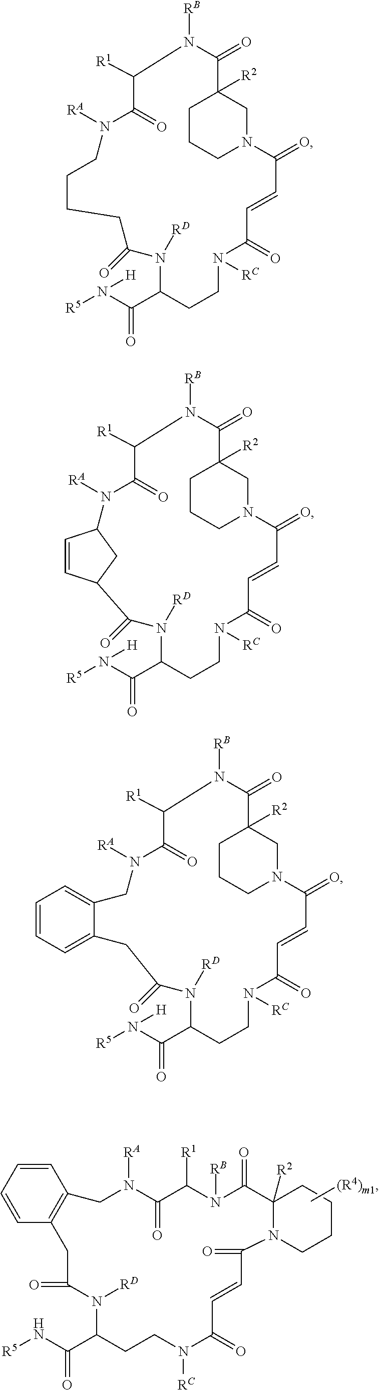

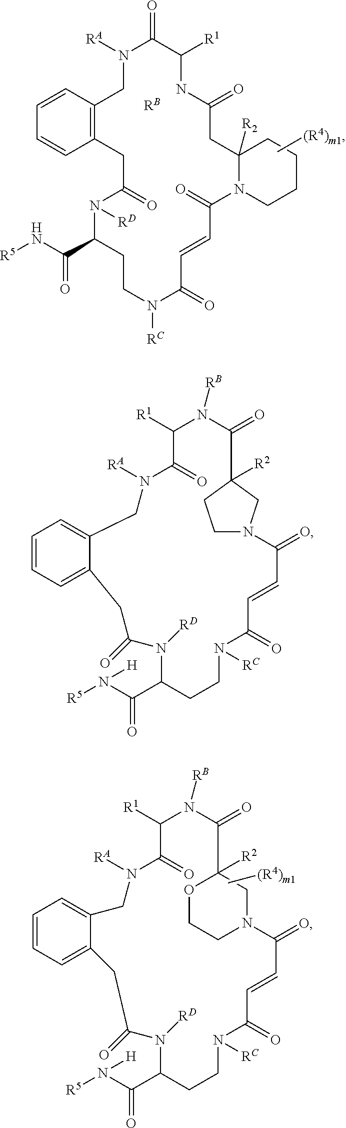

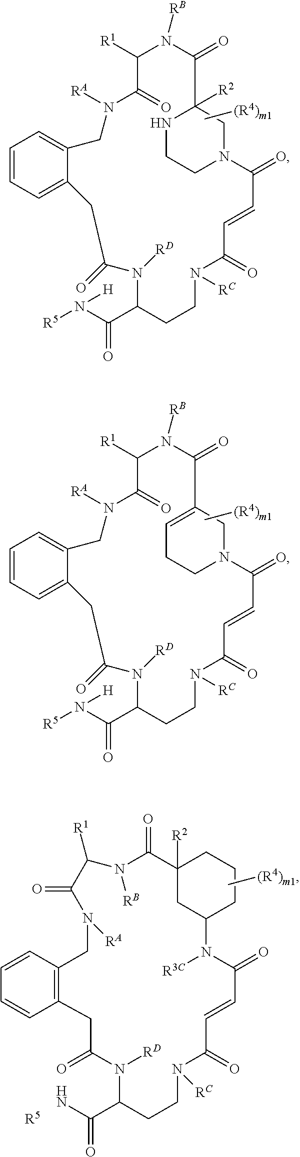

or a pharmaceutically acceptable salt, solvate, hydrate, stereoisomer, polymorph, tautomer, isotopically enriched form, or prodrug thereof.

In certain embodiments, the compound of Formula (I′) or (I) is of formula:

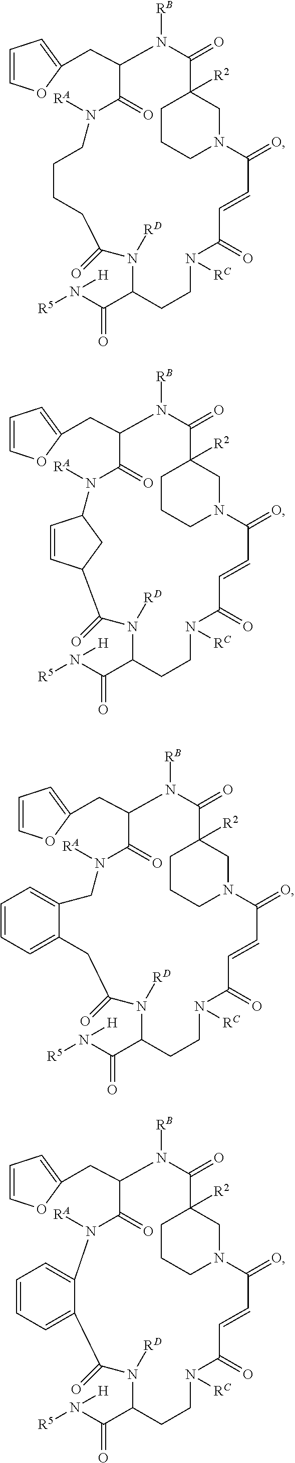

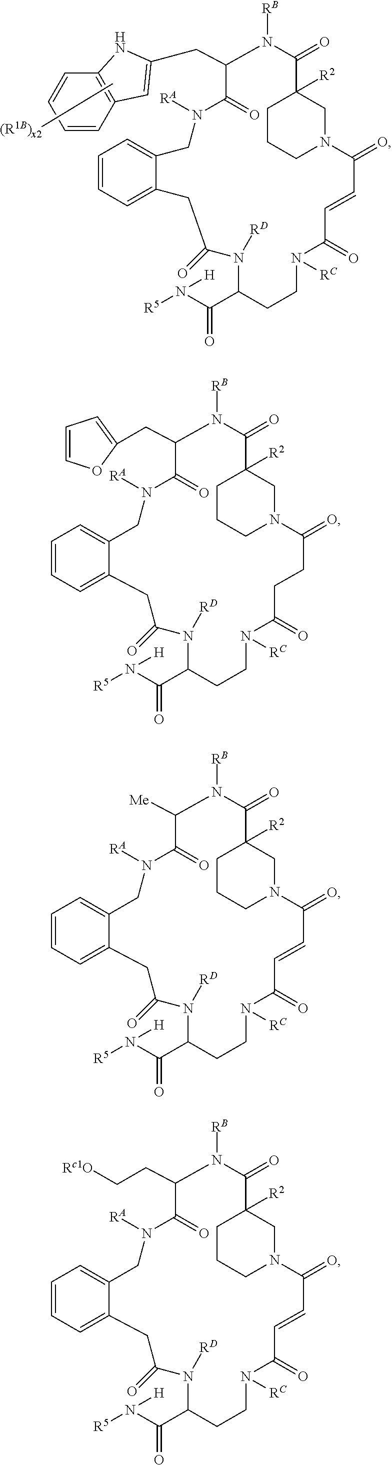

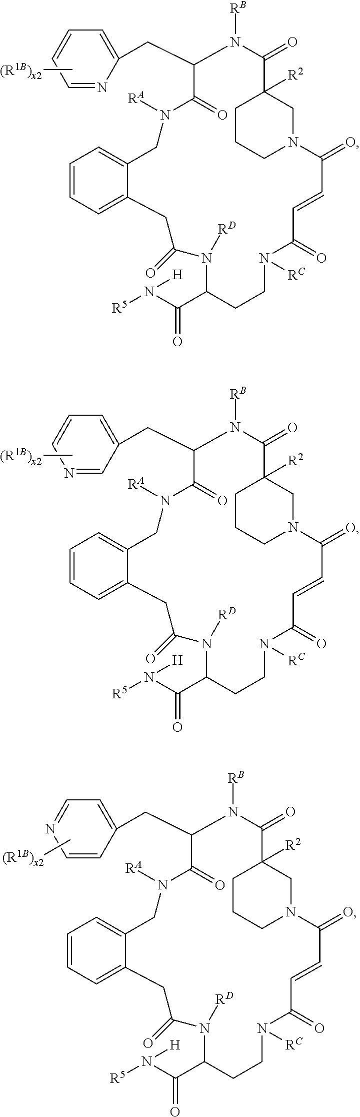

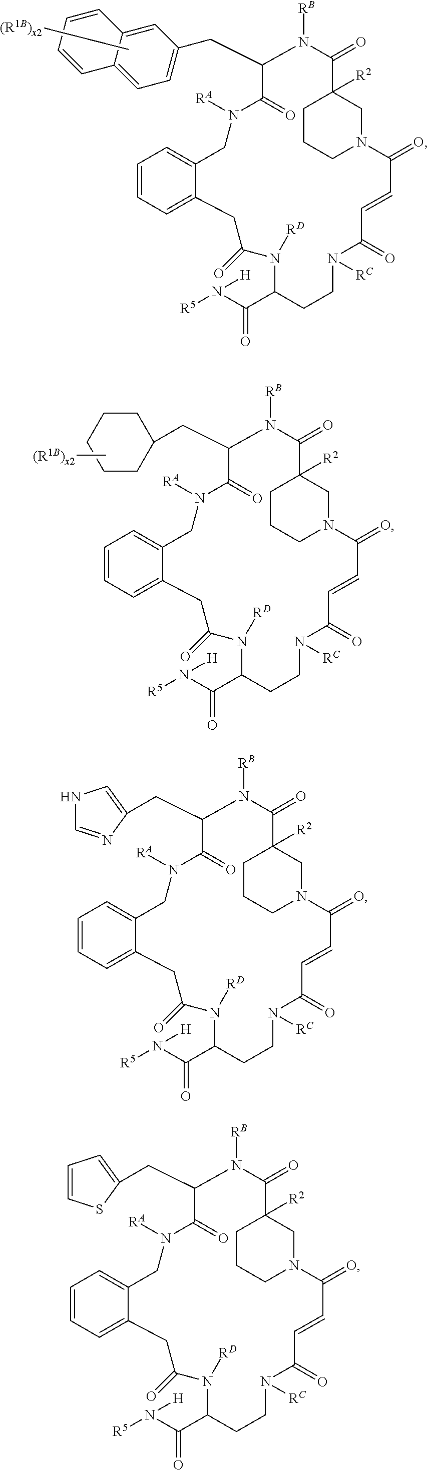

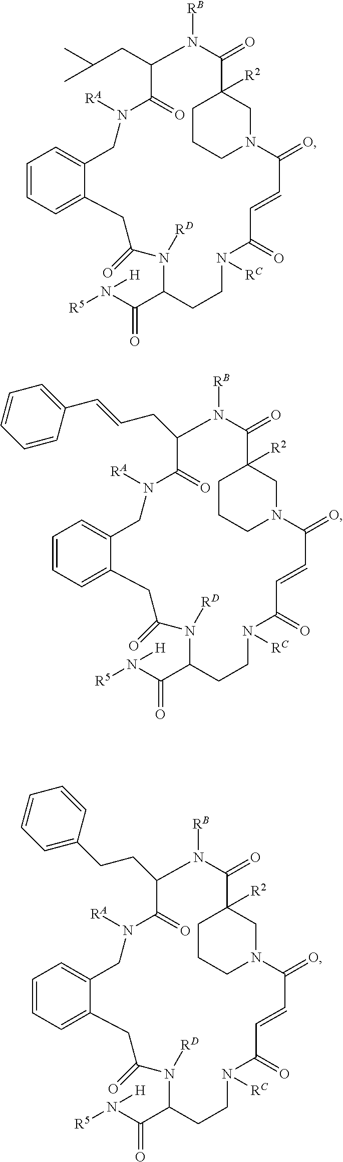

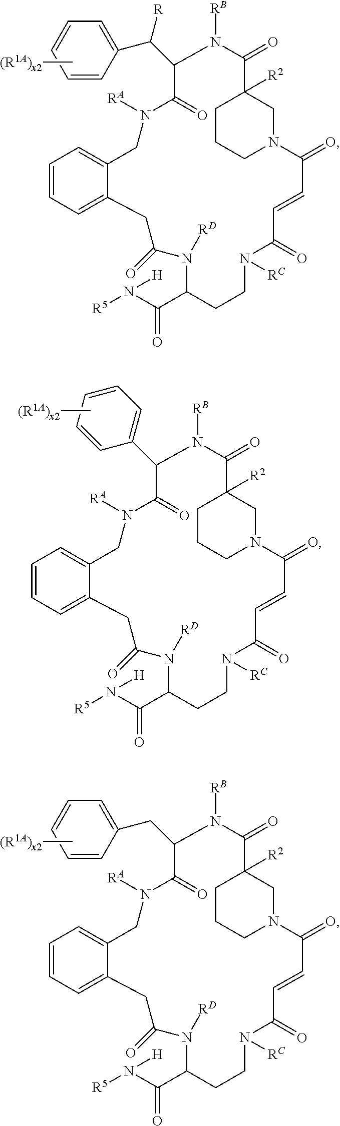

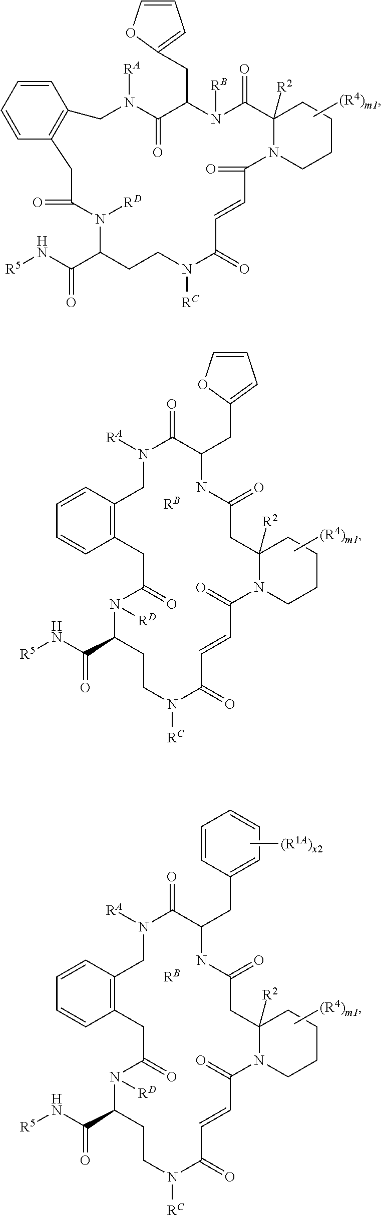

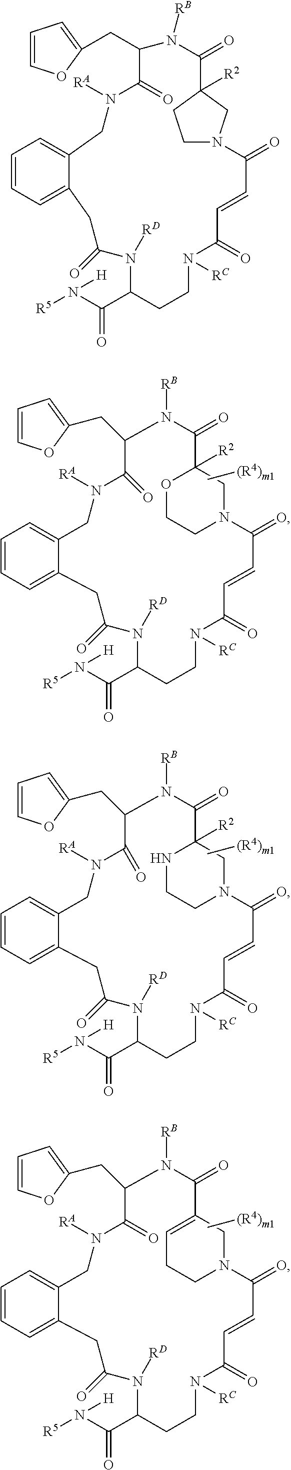

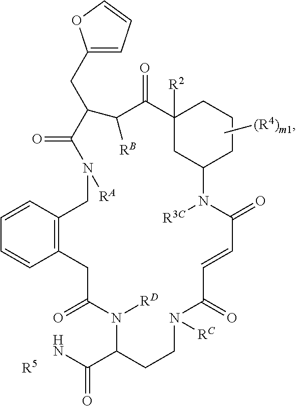

or a pharmaceutically acceptable salt, solvate, hydrate, stereoisomer, polymorph, tautomer, isotopically enriched form, or prodrug thereof.

In certain embodiments, the compound of Formula (I′) or (I) is of formula:

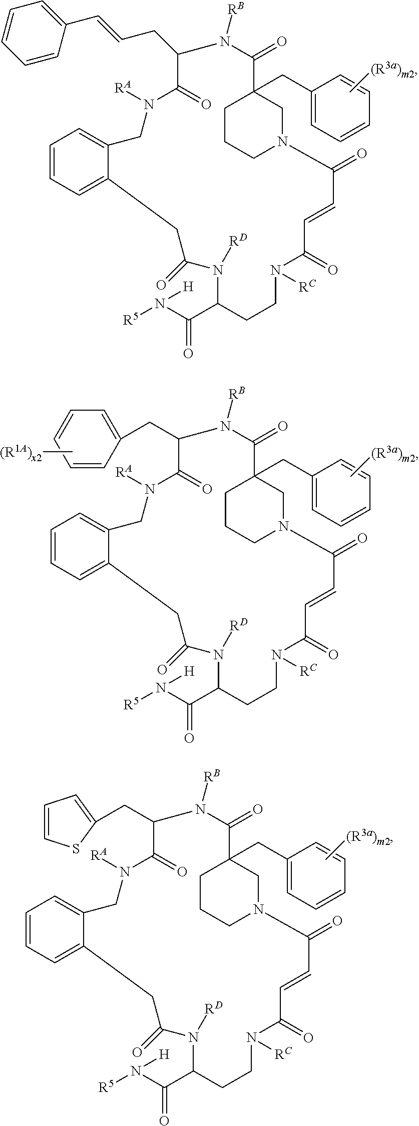

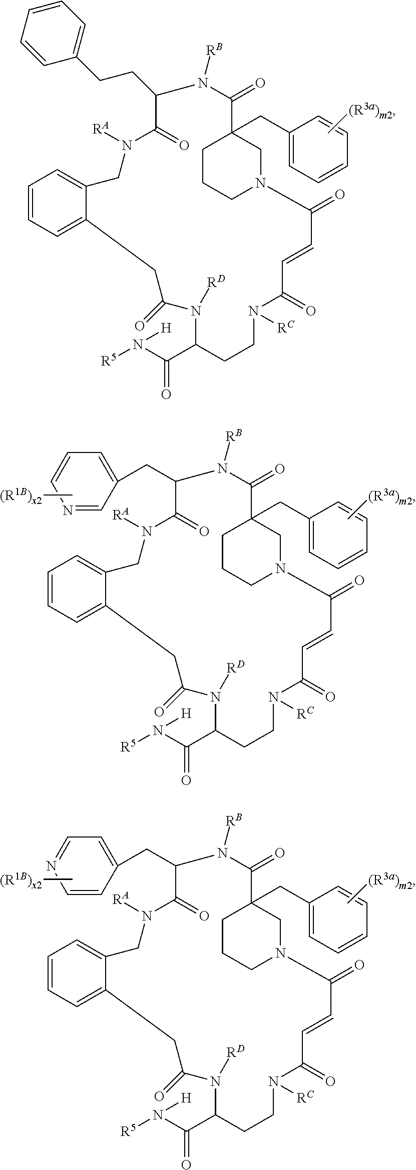

or a pharmaceutically acceptable salt, solvate, hydrate, stereoisomer, polymorph, tautomer, isotopically enriched form, or prodrug thereof.

In certain embodiments, the compound of Formula (I′) or (I) is of formula:

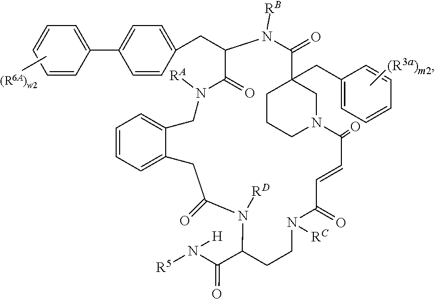

or a pharmaceutically acceptable salt, solvate, hydrate, stereoisomer, polymorph, tautomer, isotopically enriched form, or prodrug thereof.

In certain embodiments, the compound of Formula (I′) or (I) is of formula:

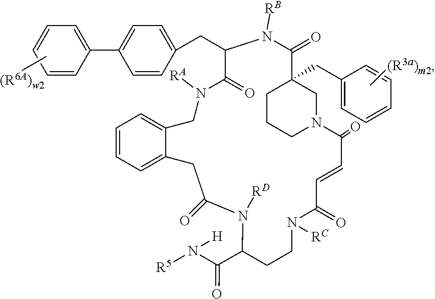

or a pharmaceutically acceptable salt, solvate, hydrate, stereoisomer, polymorph, tautomer, isotopically enriched form, or prodrug thereof.

In certain embodiments, the compound of Formula (I′) or (I) is of formula:

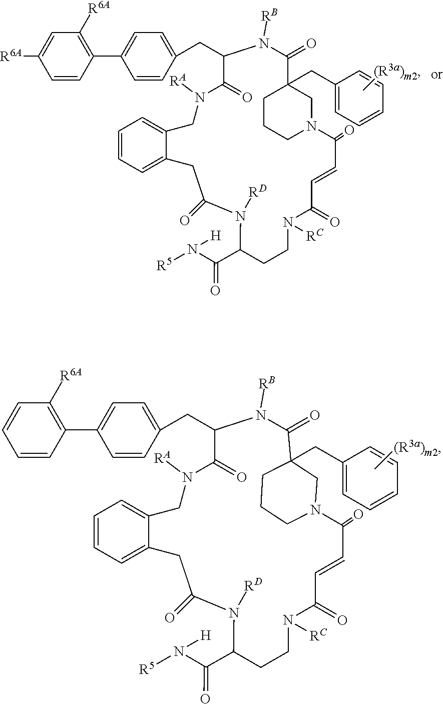

or a pharmaceutically acceptance salt, solvate, hydrate, stereoisomer, polymorph, tautomer, isotopically enriched form, or prodrug thereof, wherein each instance of R^{6A} is independently optionally substituted acyl, substituted or unsubstituted C₁₋₆ alkyl, or substituted or unsubstituted C₂₋₆ alkenyl, or —O(substituted or unsubstituted C₁₋₆ alkyl).

In certain embodiments, the compound of Formula (I′) or (I) is of formula:

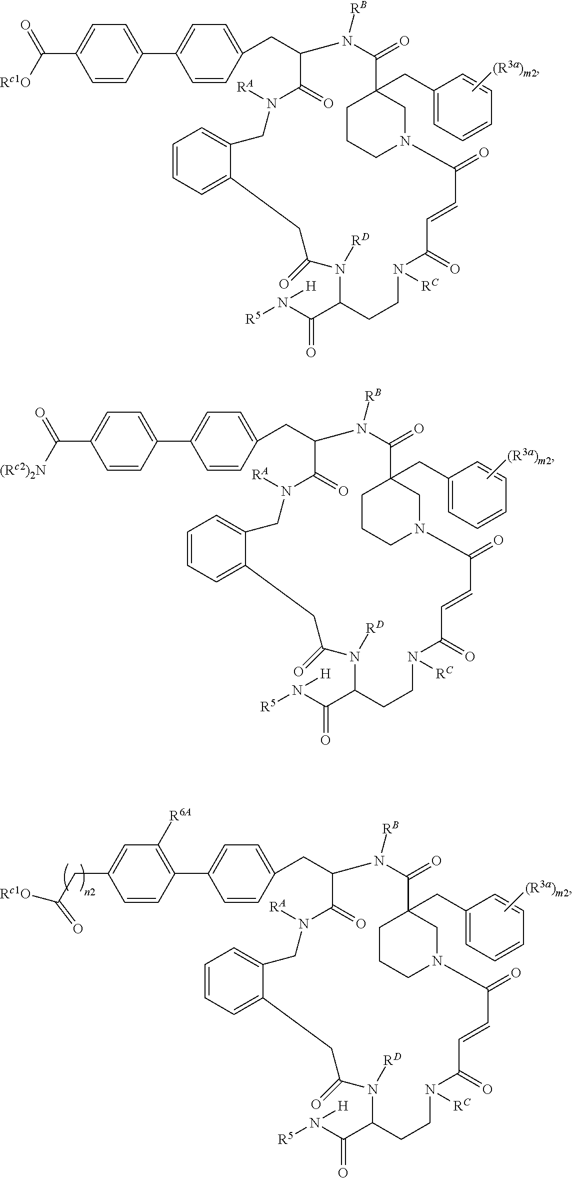

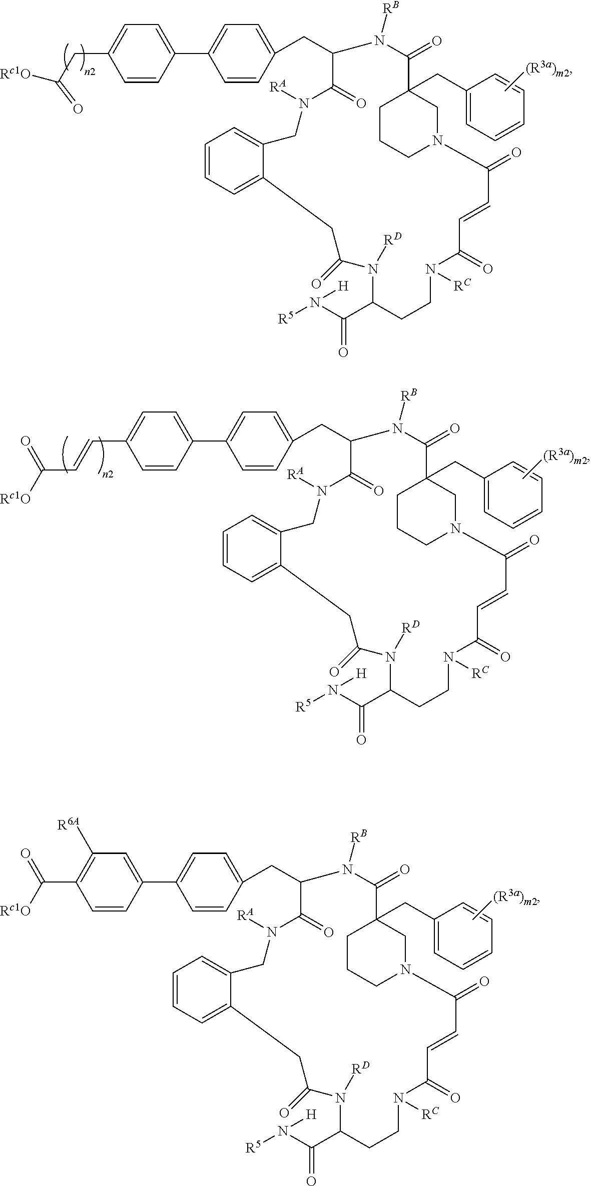

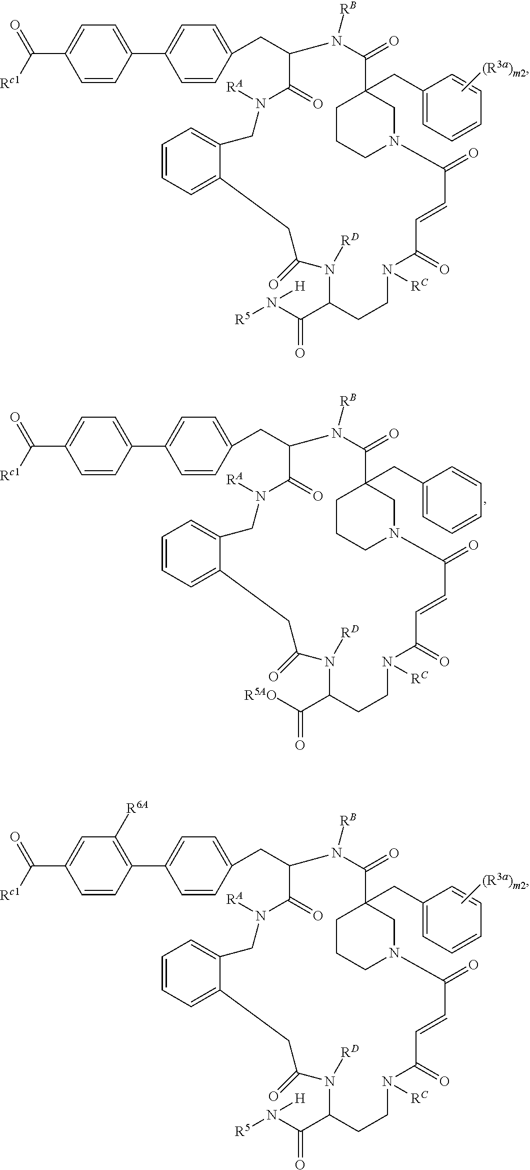

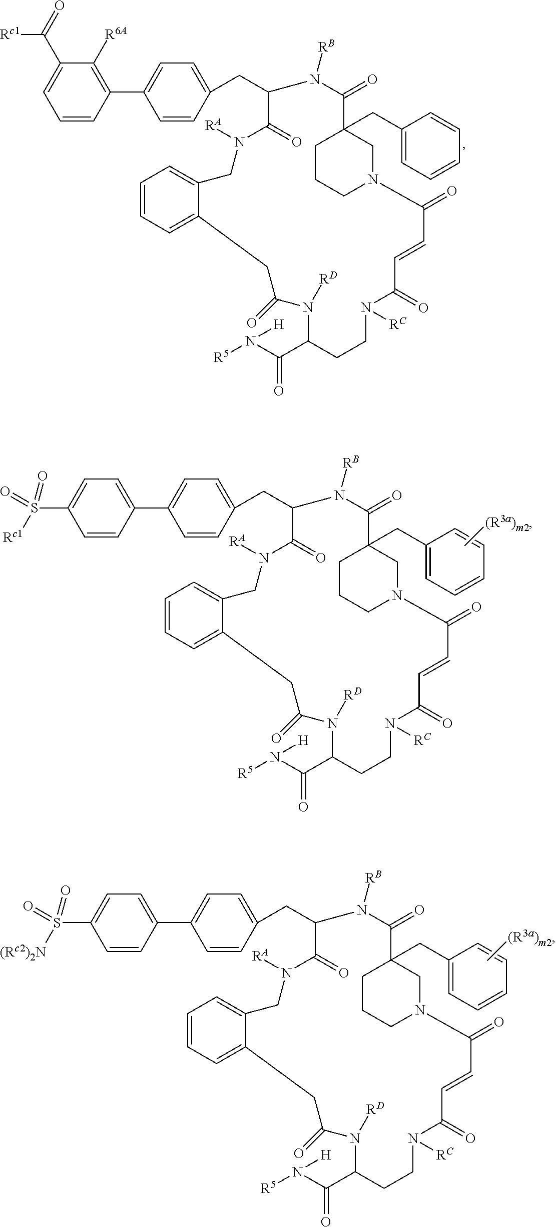

or a pharmaceutically acceptable salt, solvate, hydrate, stereoisomer, polymorph, tautomer, isotopically enriched form, or prodrug thereof, wherein n2 is 0, 1, 2, 3, 4, 5, or 6; and wherein the remaining substituents are defined as described herein.

In certain embodiments, the compound of Formula (I) or (I′) is of formula:

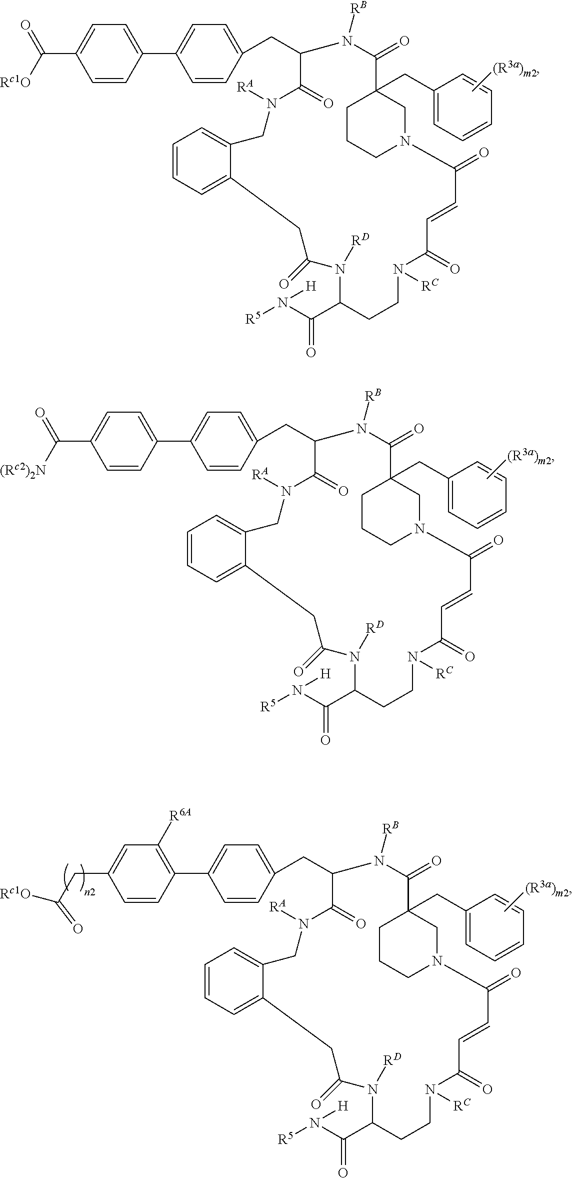

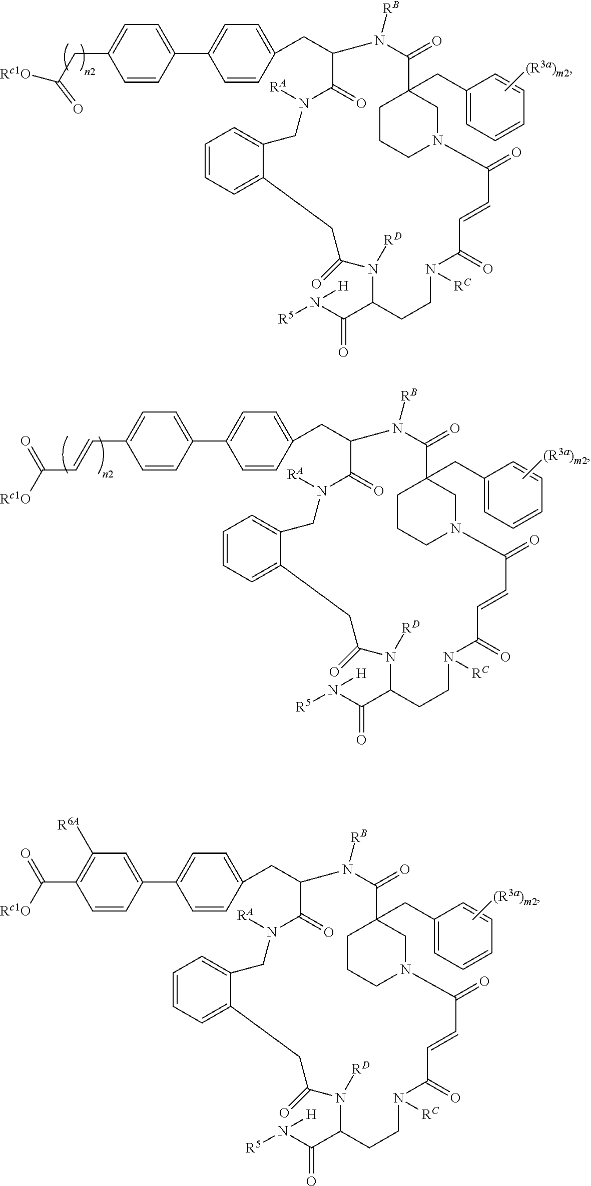

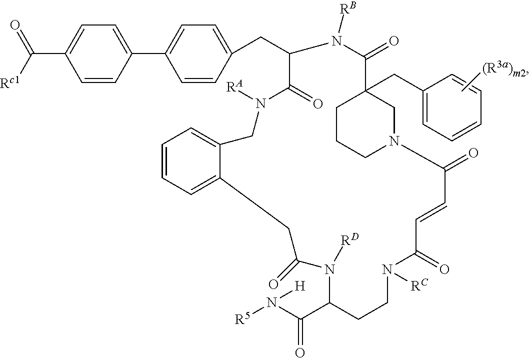

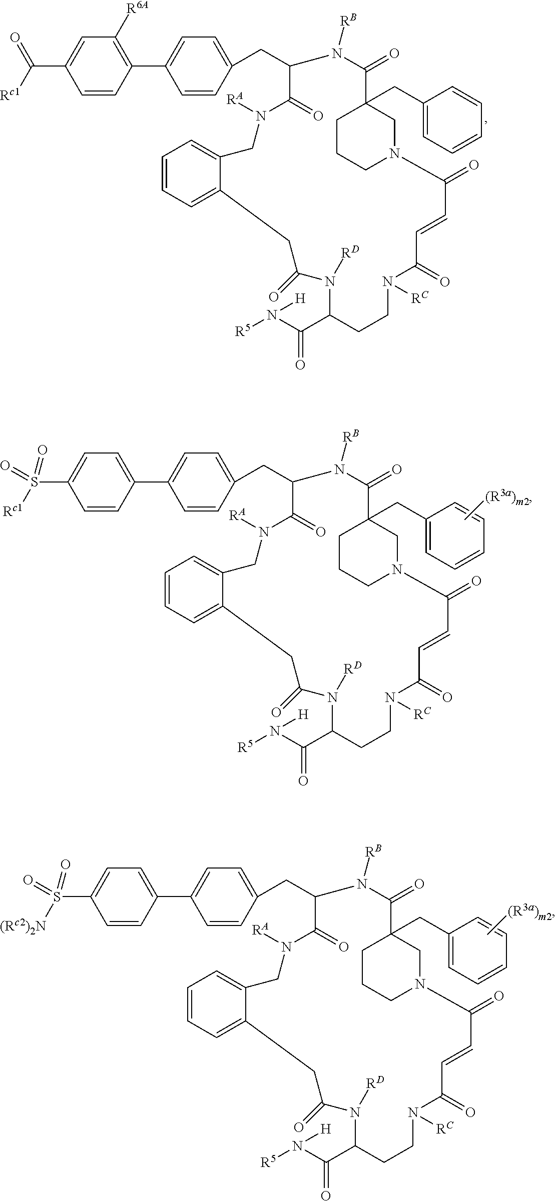

or a pharmaceutically acceptable salt, solvate, hydrate, stereoisomer, polymorph, tautomer, isotopically enriched form, or prodrug thereof, wherein n2 is 0, 1, 2, 3, 4, 5, or 6.

In certain embodiments, the compound of Formula (I′) or (I) is of formula:

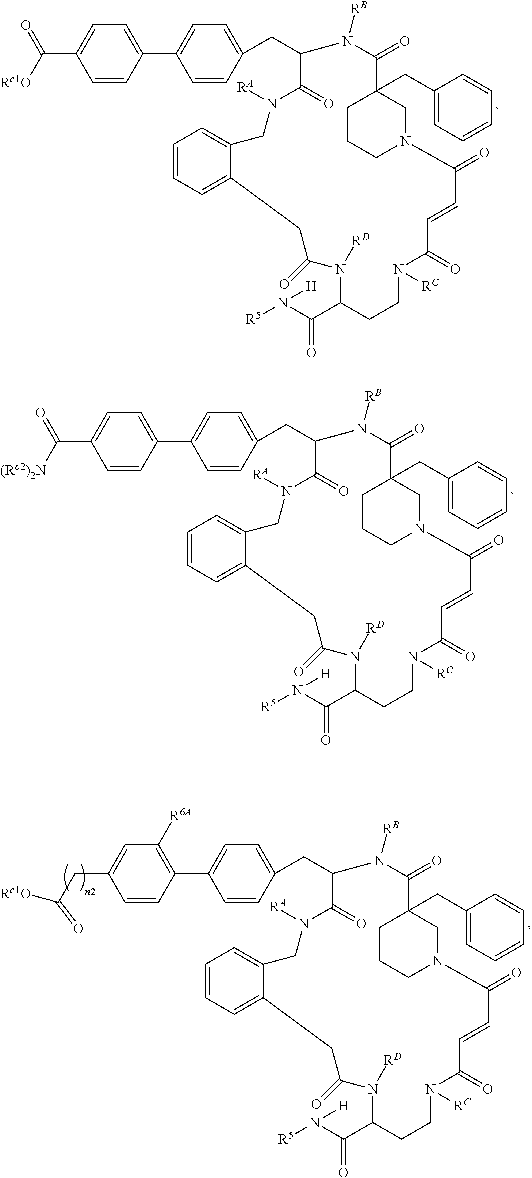

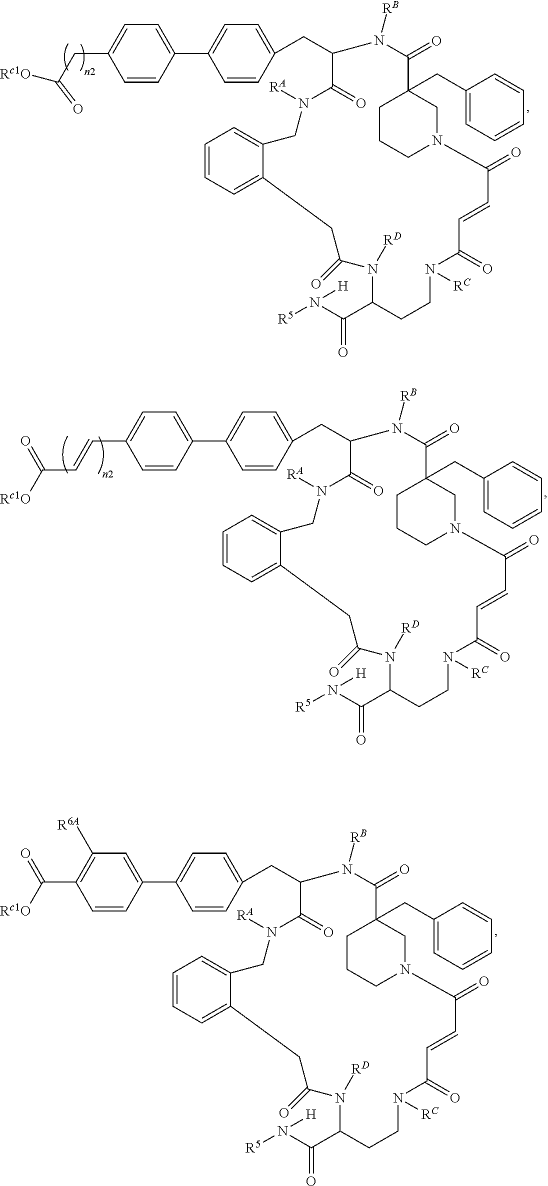

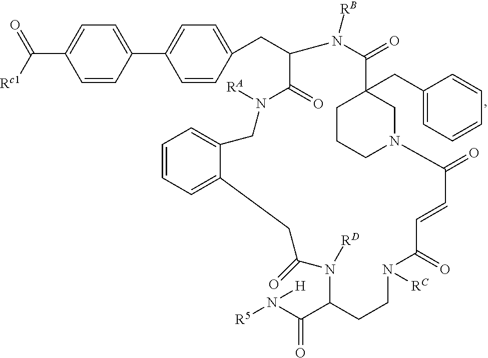

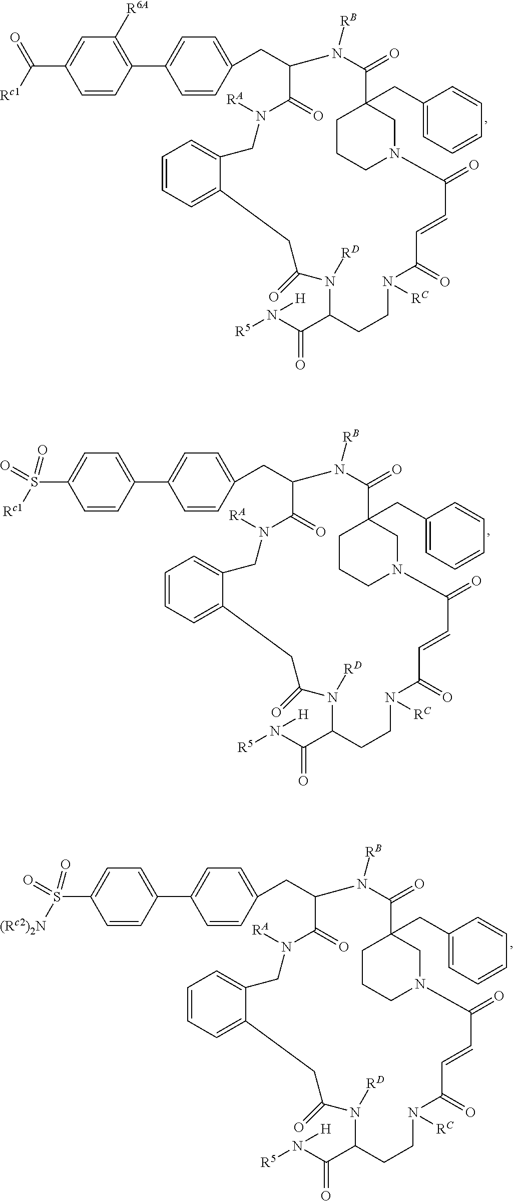

or a pharmaceutically acceptable salt, solvate, hydrate, stereoisomer, polymorph, tautomer, isotopically enriched form, or prodrug thereof, wherein n2 is 0, 1, 2, 3, 4, 5, or 6.

In certain embodiments, the compound of Formula (I′) or (I) is of formula:

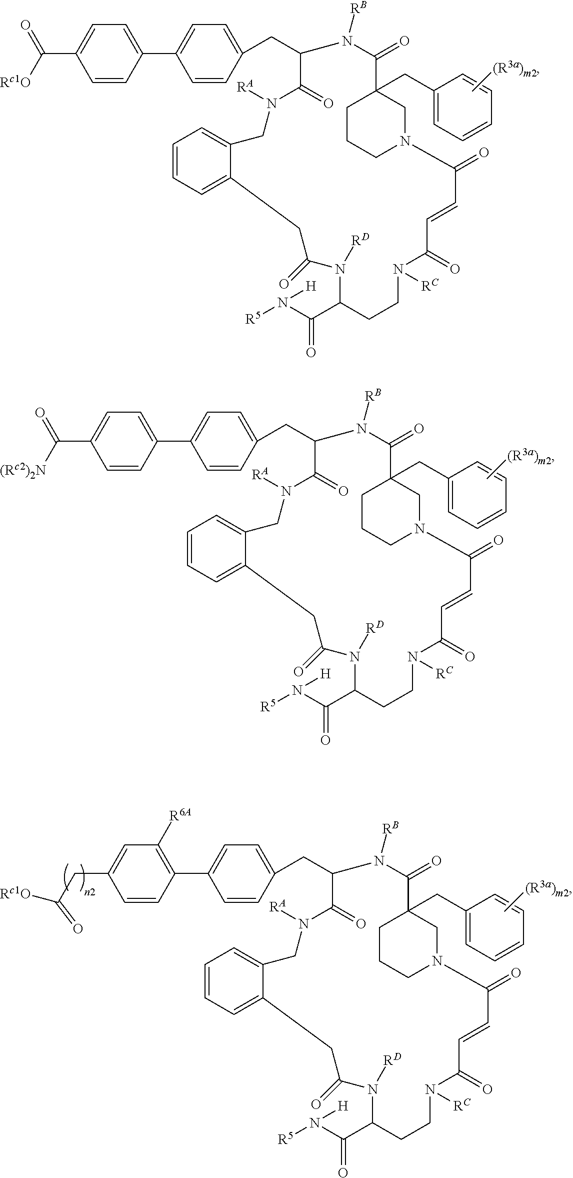

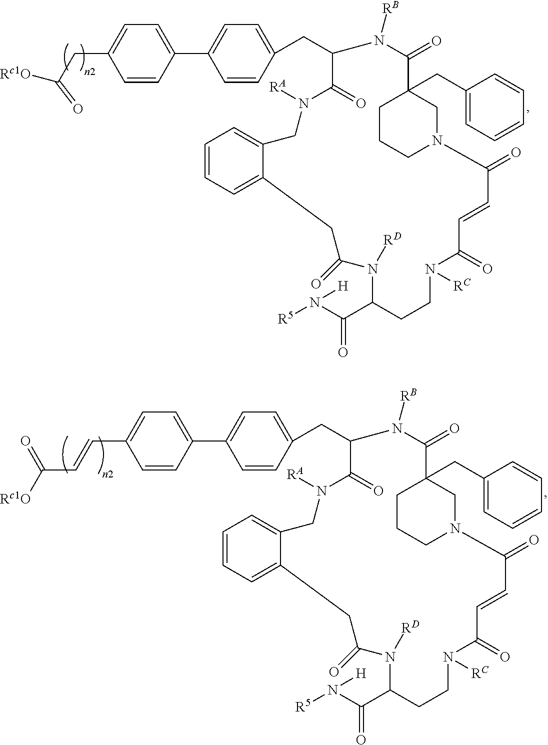

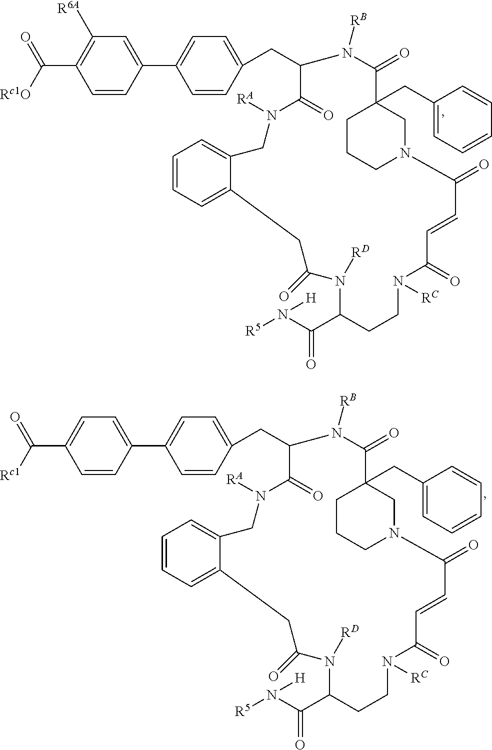

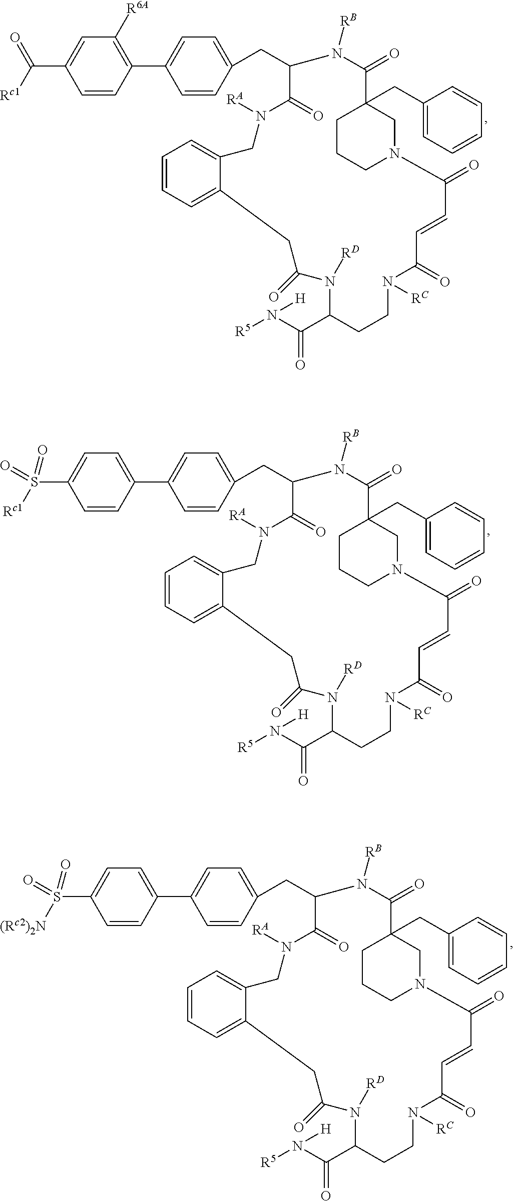

or a pharmaceutically acceptable salt, solvate, hydrate, stereoisomer, polymorph, tautomer, isotopically enriched form, or prodrug thereof, wherein n2 is 0 or 1.

In certain embodiments, the compound of Formula (I′) or (I) is of the formula:

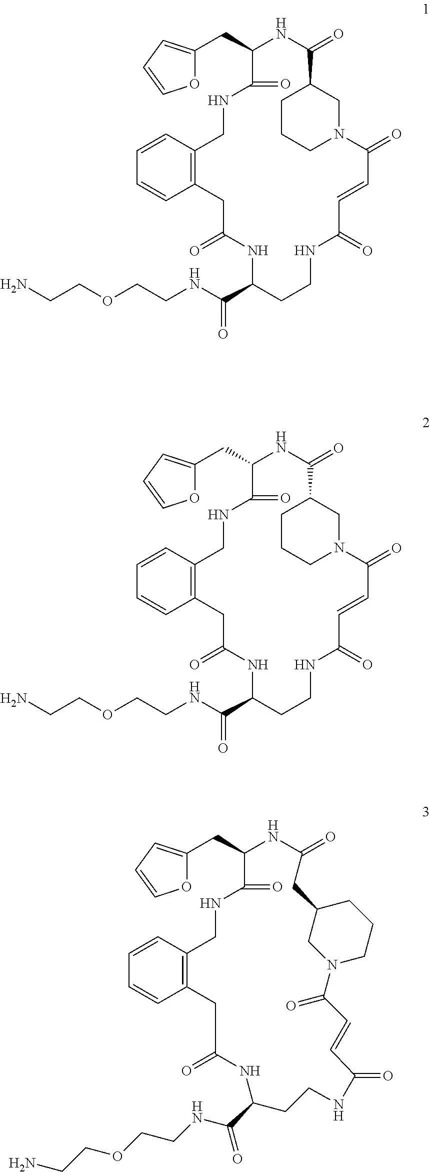 [SMILES: NCCOCCNC(=O)[C@@H]1CCNC(=O)/C=C/C(=O)N2CCC[C@H](C2)C(=O)N[C@H](Cc2ccco2)C(=O)NCc2ccccc2CC(=O)N1 ; NCCOCCNC(=O)[C@@H]1CCNC(=O)/C=C/C(=O)N2CCC[C@@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@H](CC(=O)N[C@H](Cc3ccco3)C(=O)NCc3ccccc3CC(=O)N1)C2]

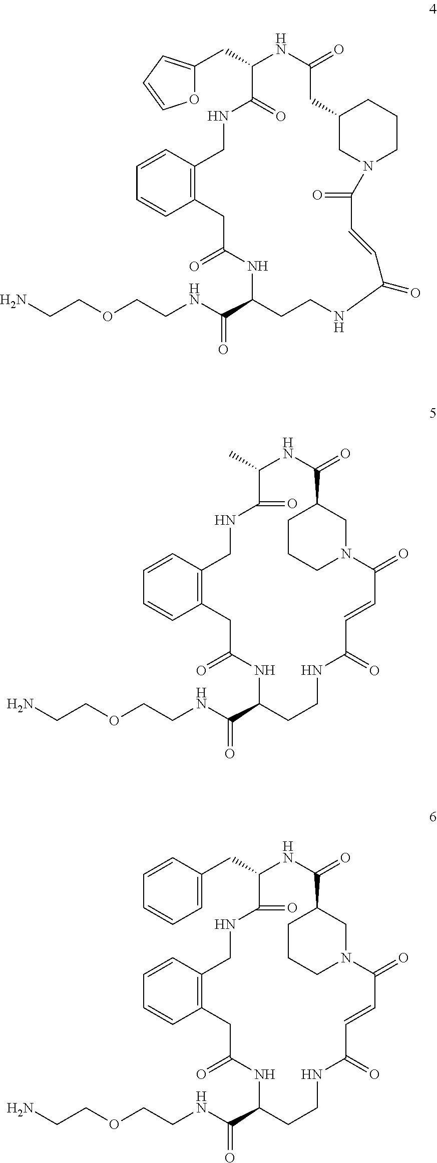 [SMILES: NCCOCCNC(=O)[C@@H]1CCNC(=O)/C=C/C(=O)N2CCC[C@@H](CC(=O)N[C@@H](Cc3ccco3)C(=O)NCc3ccccc3CC(=O)N1)C2 ; C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccccc2)C(=O)NCc2ccccc2CC(=O)N1]

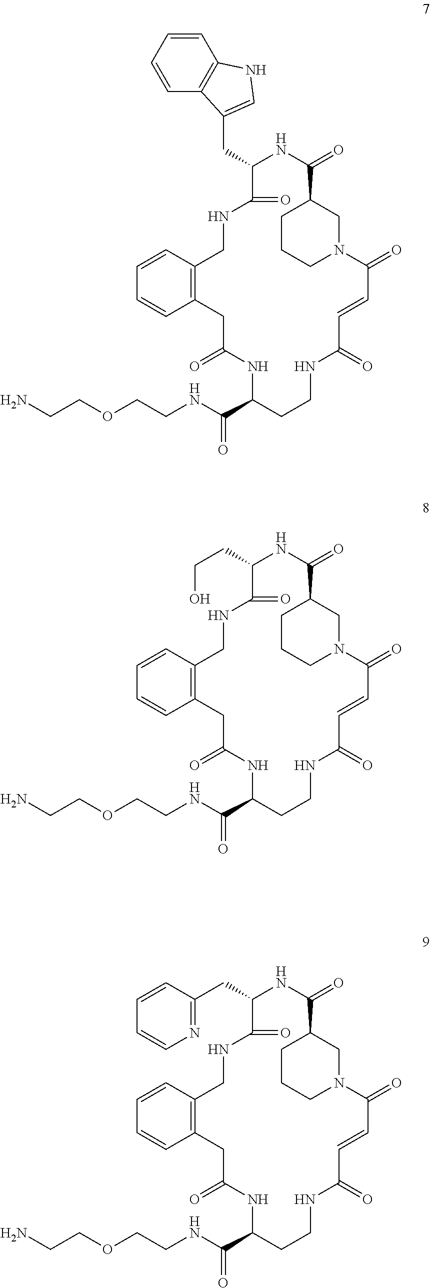 [SMILES: NCCOCCNC(=O)[C@@H]1CCNC(=O)/C=C/C(=O)N2CCC[C@H](C2)C(=O)N[C@@H](Cc2c[nH]c3ccccc23)C(=O)NCc2ccccc2CC(=O)N1 ; NCCOCCNC(=O)[C@@H]1CCNC(=O)/C=C/C(=O)N2CCC[C@H](C2)C(=O)N[C@@H](CCO)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccccn2)C(=O)NCc2ccccc2CC(=O)N1]

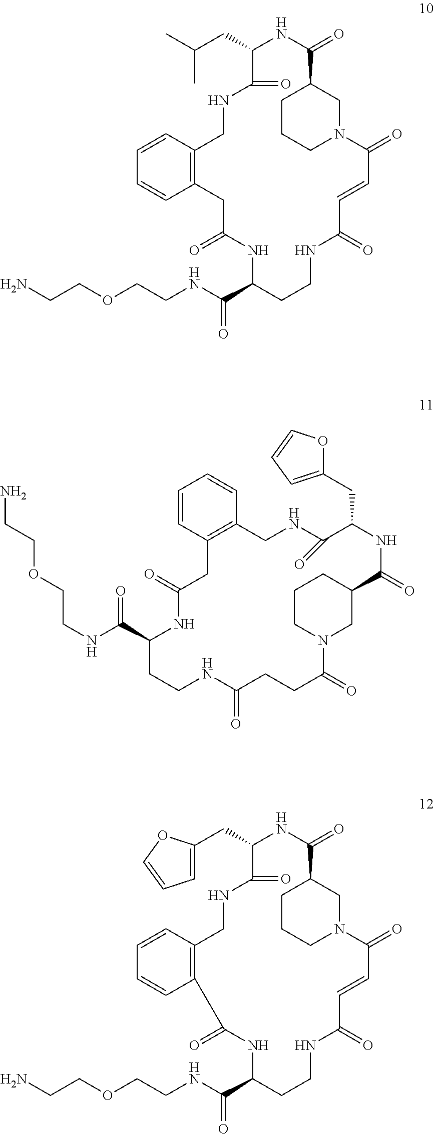 [SMILES: CC(C)C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)CCC(=O)N2CCC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccco2)C(=O)NCc2ccccc2C(=O)N1]

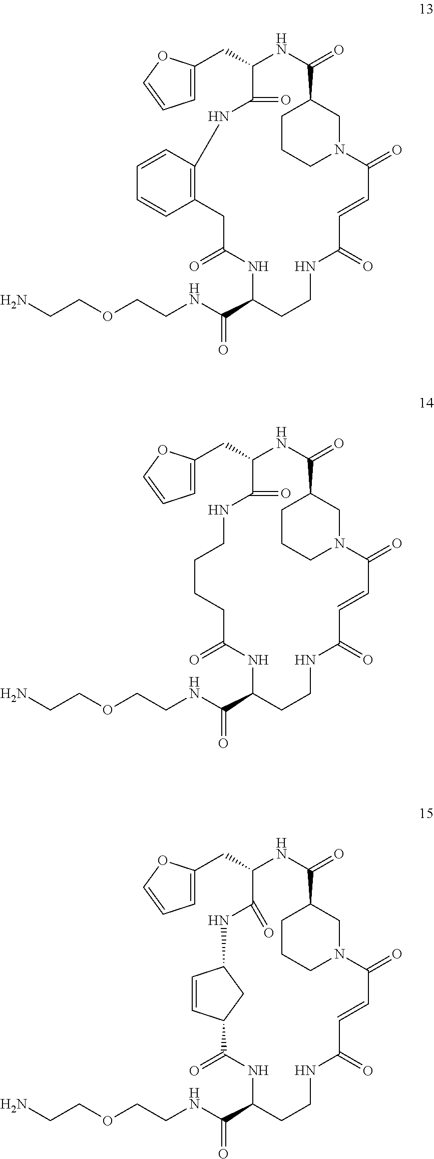 [SMILES: NCCOCCNC(=O)[C@@H]1CCNC(=O)/C=C/C(=O)N2CCC[C@H](C2)C(=O)N[C@@H](Cc2ccco2)C(=O)Nc2ccccc2CC(=O)N1 ; NCCOCCNC(=O)[C@@H]1CCNC(=O)/C=C/C(=O)N2CCC[C@H](C2)C(=O)N[C@@H](Cc2ccco2)C(=O)NCCCCC(=O)N1 ; NCCOCCNC(=O)[C@@H]1CCNC(=O)/C=C/C(=O)N2CCC[C@H](C2)C(=O)N[C@@H](Cc2ccco2)C(=O)N[C@H]2C=C[C@H](C2)C(=O)N1]

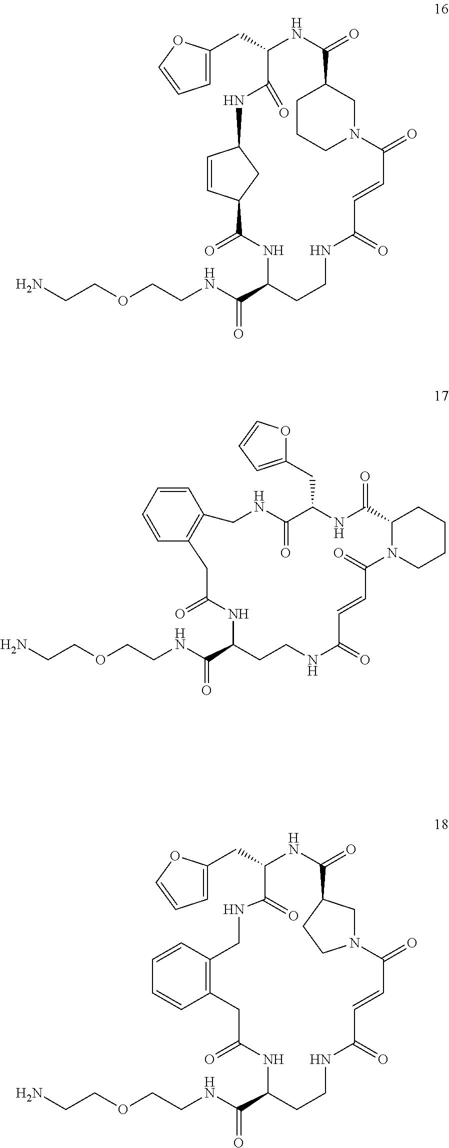 [SMILES: NCCOCCNC(=O)[C@@H]1CCNC(=O)/C=C/C(=O)N2CCC[C@H](C2)C(=O)N[C@@H](Cc2ccco2)C(=O)N[C@@H]2C=C[C@@H](C2)C(=O)N1 ; NCCOCCNC(=O)[C@@H]1CCNC(=O)/C=C/C(=O)N2CCCC[C@H]2C(=O)N[C@@H](Cc2ccco2)C(=O)NCc2ccccc2CC(=O)N1 ; NCCOCCNC(=O)[C@@H]1CCNC(=O)/C=C/C(=O)N2CC[C@H](C2)C(=O)N[C@@H](Cc2ccco2)C(=O)NCc2ccccc2CC(=O)N1]

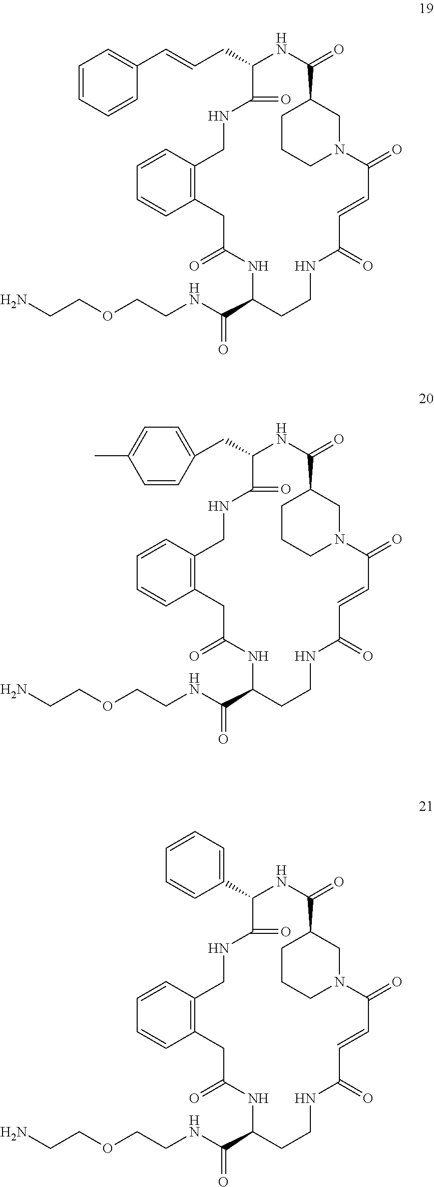 [SMILES: NCCOCCNC(=O)[C@@H]1CCNC(=O)/C=C/C(=O)N2CCC[C@H](C2)C(=O)N[C@@H](C/C=C/c2ccccc2)C(=O)NCc2ccccc2CC(=O)N1 ; Cc1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@H](C2)C(=O)N[C@@H](c2ccccc2)C(=O)NCc2ccccc2CC(=O)N1]

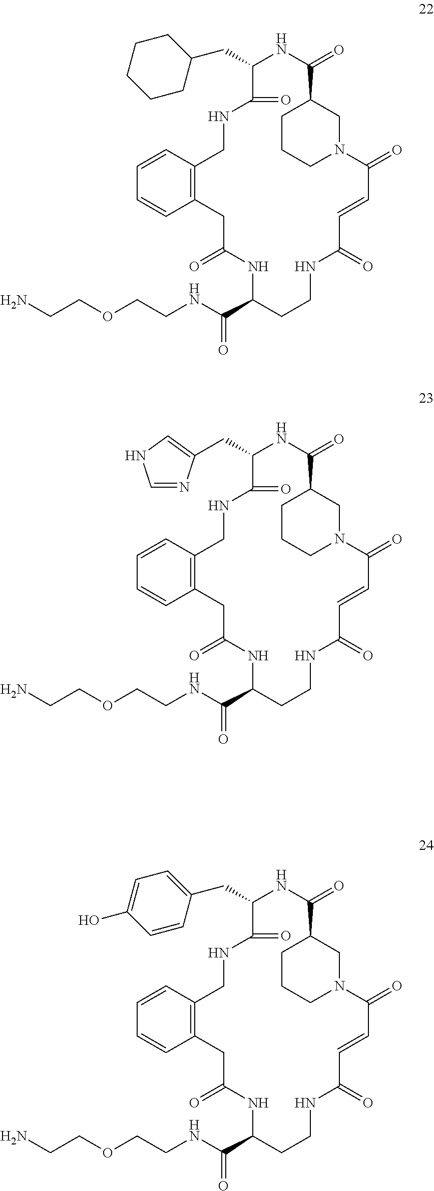 [SMILES: NCCOCCNC(=O)[C@@H]1CCNC(=O)/C=C/C(=O)N2CCC[C@H](C2)C(=O)N[C@@H](CC2CCCCC2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2c[nH]cn2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(O)cc2)C(=O)NCc2ccccc2CC(=O)N1]

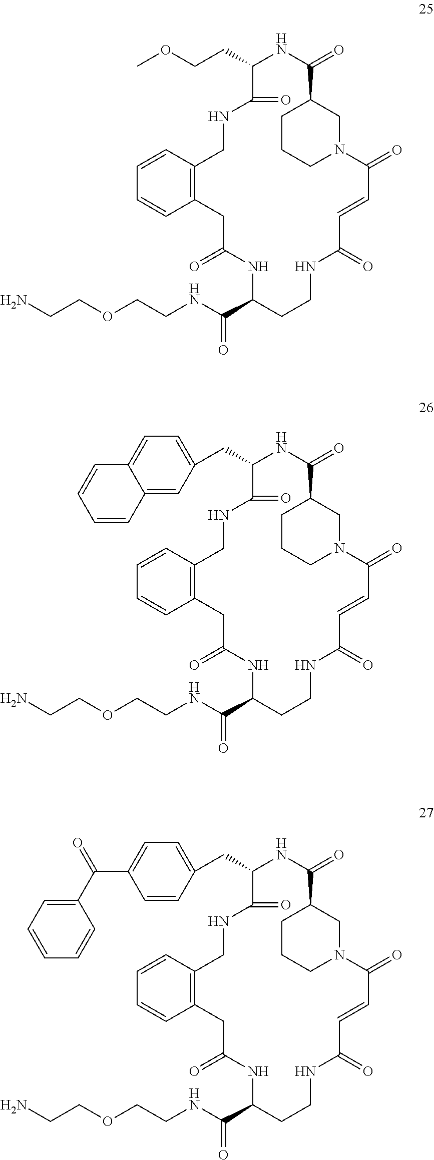 [SMILES: COCC[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccc3ccccc3c2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(C(=O)c3ccccc3)cc2)C(=O)NCc2ccccc2CC(=O)N1]

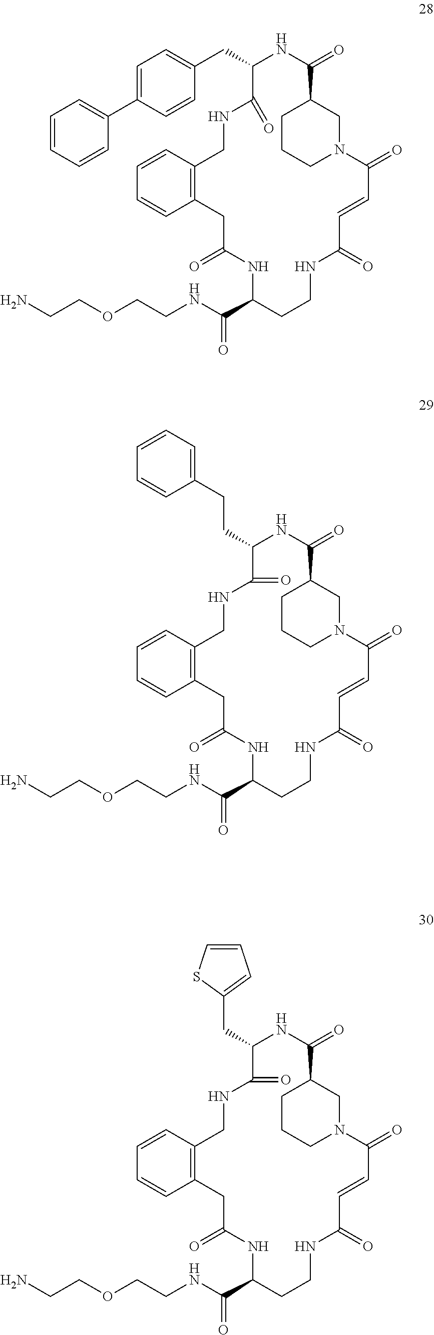

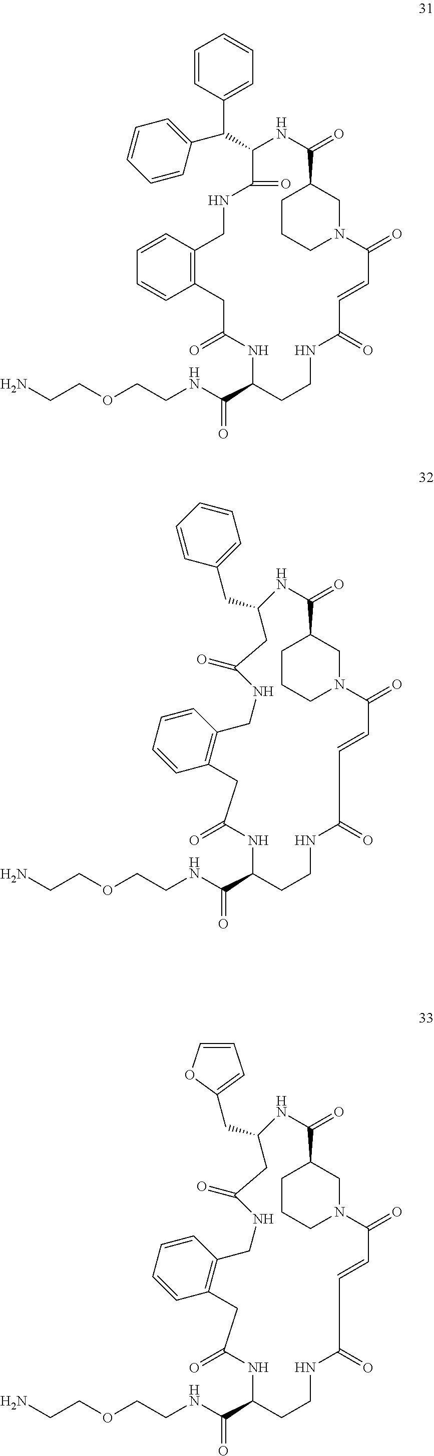 [SMILES: NCCOCCNC(=O)[C@@H]1CCNC(=O)/C=C/C(=O)N2CCC[C@H](C2)C(=O)N[C@@H](C(c2ccccc2)c2ccccc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccccc2)CC(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccco2)CC(=O)NCc2ccccc2CC(=O)N1]

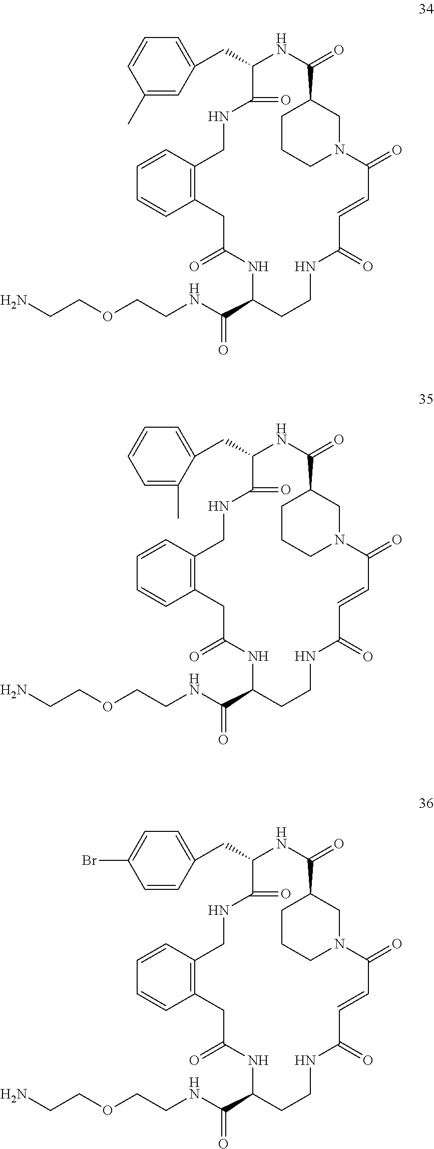 [SMILES: Cc1cccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)c1 ; Cc1ccccc1C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccc(Br)cc2)C(=O)NCc2ccccc2CC(=O)N1]

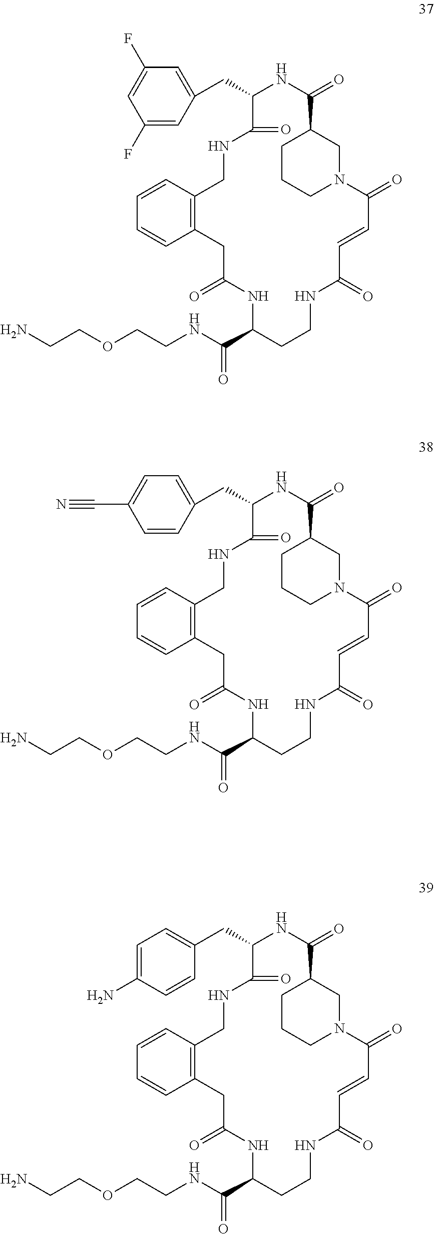 [SMILES: NCCOCCNC(=O)[C@@H]1CCNC(=O)/C=C/C(=O)N2CCC[C@H](C2)C(=O)N[C@@H](Cc2cc(F)cc(F)c2)C(=O)NCc2ccccc2CC(=O)N1 ; N#Cc1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@H](C2)C(=O)N[C@@H](Cc2ccc(N)cc2)C(=O)NCc2ccccc2CC(=O)N1]

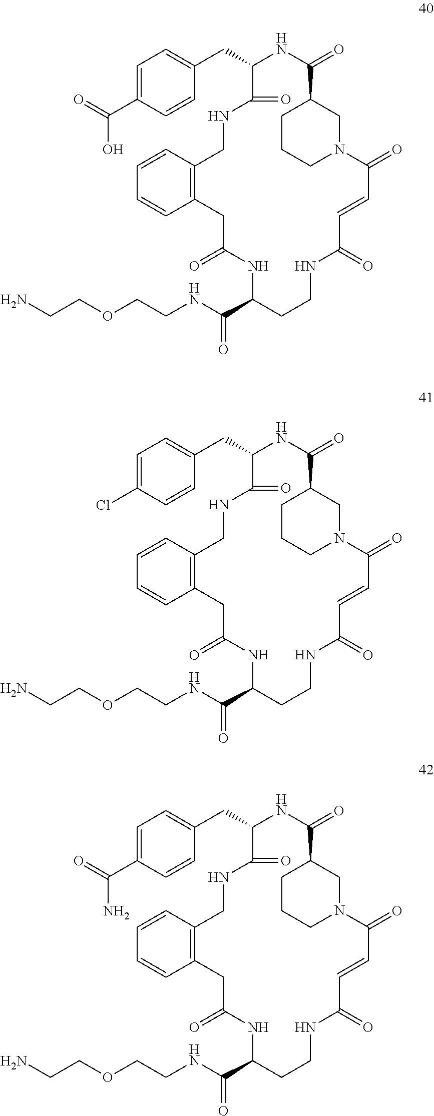 [SMILES: NCCOCCNC(=O)[C@@H]1CCNC(=O)/C=C/C(=O)N2CCC[C@H](C2)C(=O)N[C@@H](Cc2ccc(C(=O)O)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(Cl)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(C(N)=O)cc2)C(=O)NCc2ccccc2CC(=O)N1]

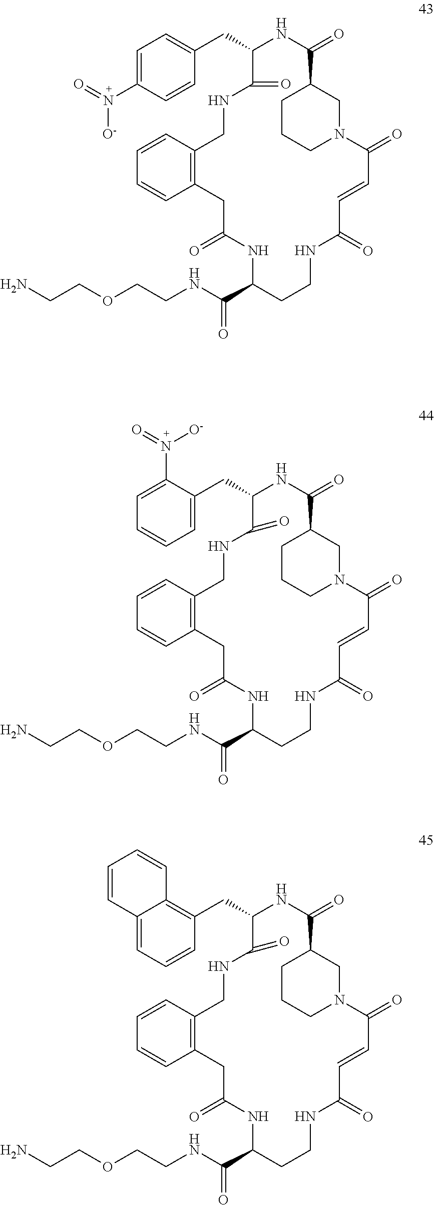

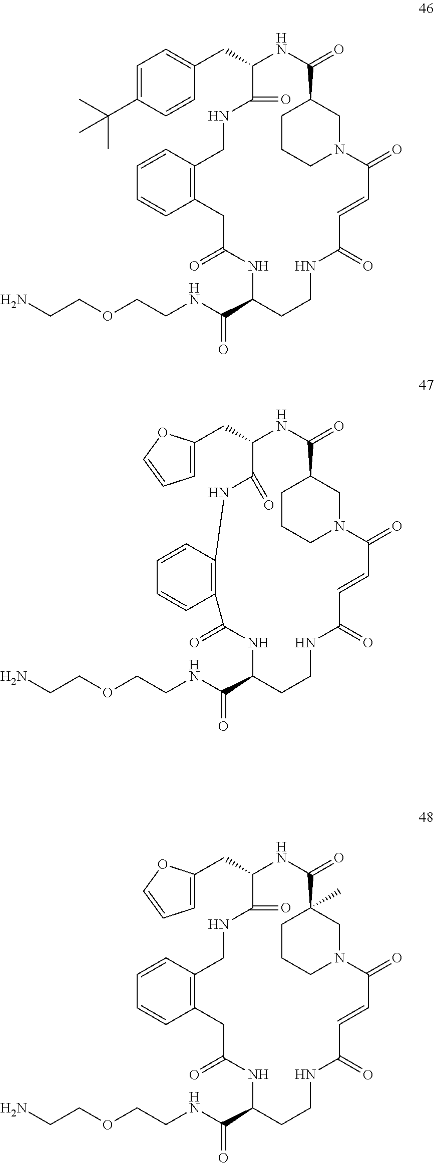 [SMILES: CC(C)(C)c1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@H](C2)C(=O)N[C@@H](Cc2ccco2)C(=O)Nc2ccccc2C(=O)N1 ; C[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

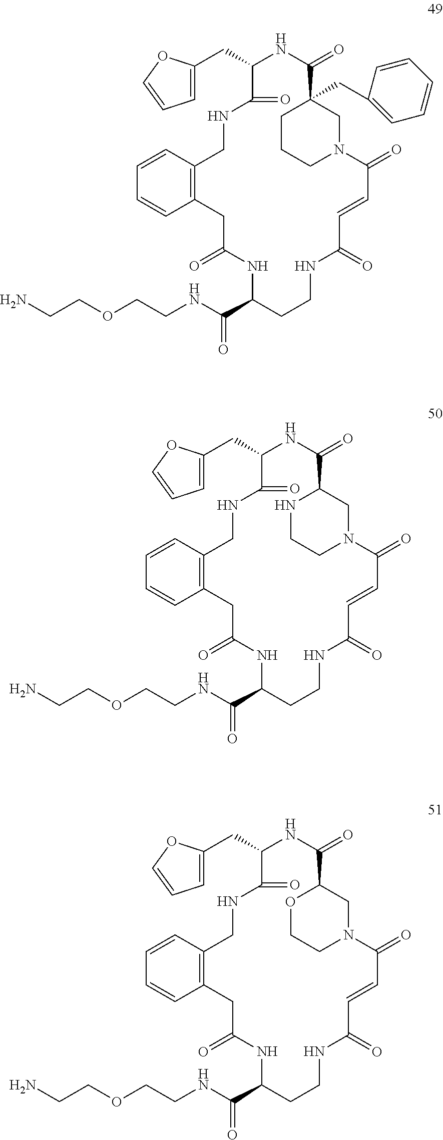 [SMILES: NCCOCCNC(=O)[C@@H]1CCNC(=O)/C=C/C(=O)N2CCC[C@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCN[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCO[C@H](C2)C(=O)N[C@@H](Cc2ccco2)C(=O)NCc2ccccc2CC(=O)N1]

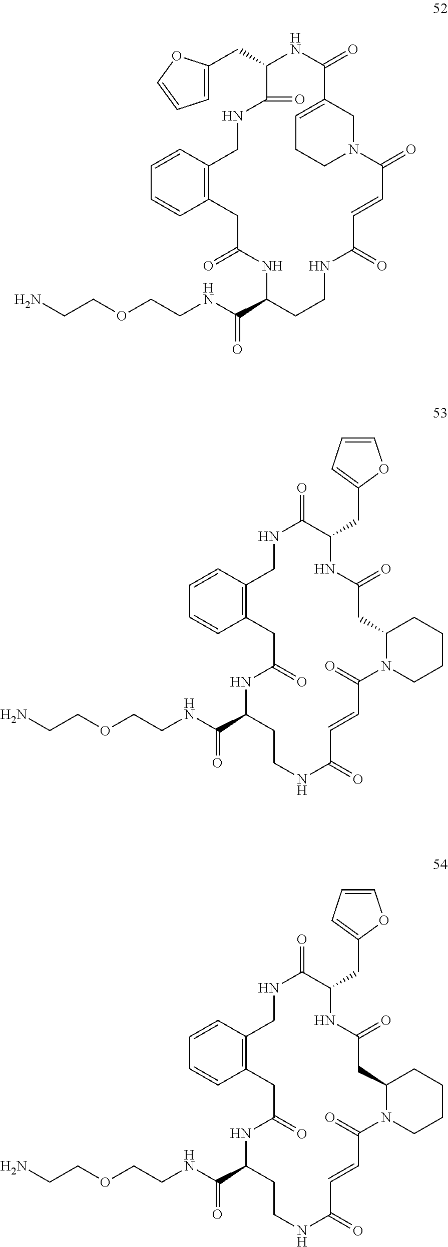 [SMILES: NCCOCCNC(=O)[C@@H]1CCNC(=O)/C=C/C(=O)N2CCC=C(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCCC[C@H]2CC(=O)N[C@@H](Cc2ccco2)C(=O)NCc2ccccc2CC(=O)N1 ; NCCOCCNC(=O)[C@@H]1CCNC(=O)/C=C/C(=O)N2CCCC[C@@H]2CC(=O)N[C@@H](Cc2ccco2)C(=O)NCc2ccccc2CC(=O)N1]

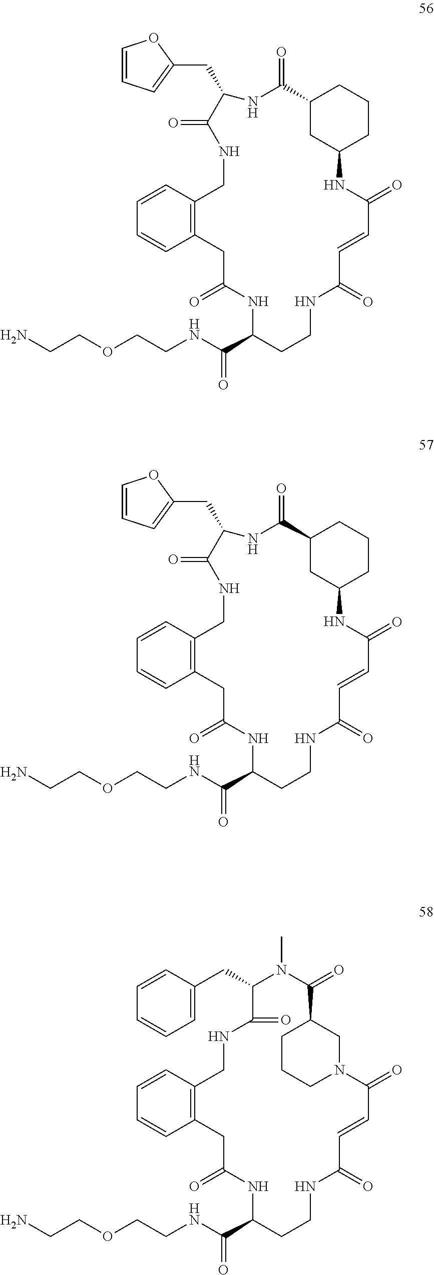 [SMILES: NCCOCCNC(=O)[C@@H]1CCNC(=O)/C=C/C(=O)N[C@@H]2CCC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N[C@@H]2CCC[C@@H](C2)C(=O)N[C@@H](Cc2ccco2)C(=O)NCc2ccccc2CC(=O)N1 ; CN1C(=O)[C@@H]2CCCN(C2)C(=O)/C=C/C(=O)NCC[C@@H](C(=O)NCCOCCN)NC(=O)Cc2ccccc2CNC(=O)[C@@H]1Cc1ccccc1]

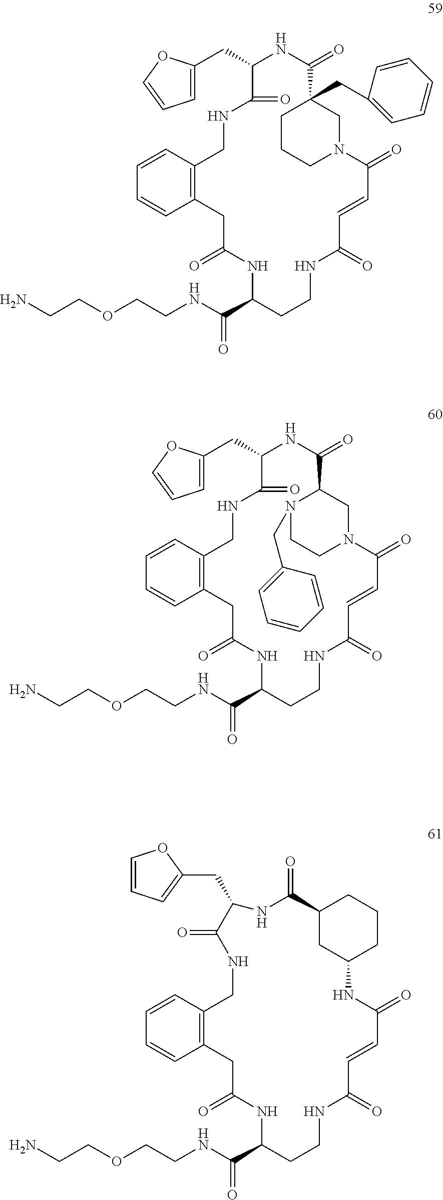 [SMILES: NCCOCCNC(=O)[C@@H]1CCNC(=O)/C=C/C(=O)N2CCC[C@@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCN(Cc3ccccc3)[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N[C@H]2CCC[C@@H](C2)C(=O)N[C@@H](Cc2ccco2)C(=O)NCc2ccccc2CC(=O)N1]

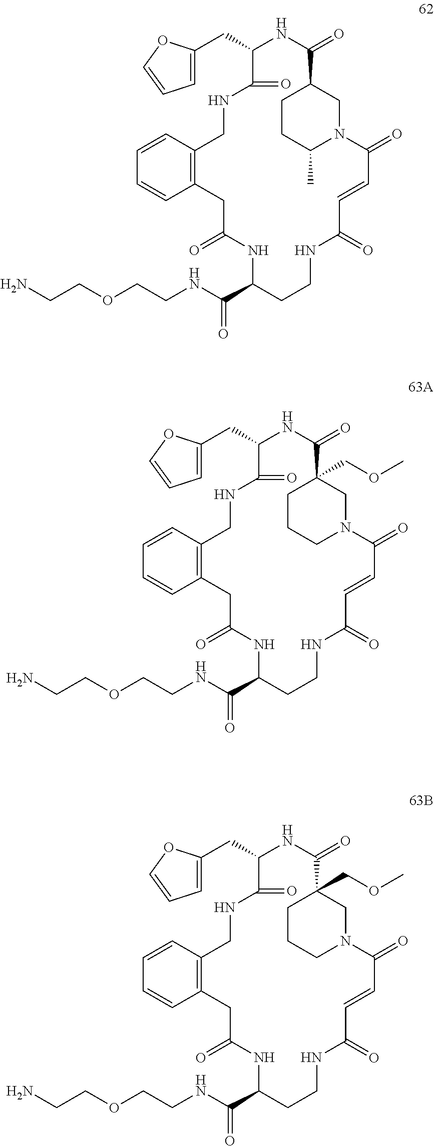 [SMILES: C[C@@H]1CC[C@@H]2CN1C(=O)/C=C/C(=O)NCC[C@@H](C(=O)NCCOCCN)NC(=O)Cc1ccccc1CNC(=O)[C@H](Cc1ccco1)NC2=O ; COC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; COC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

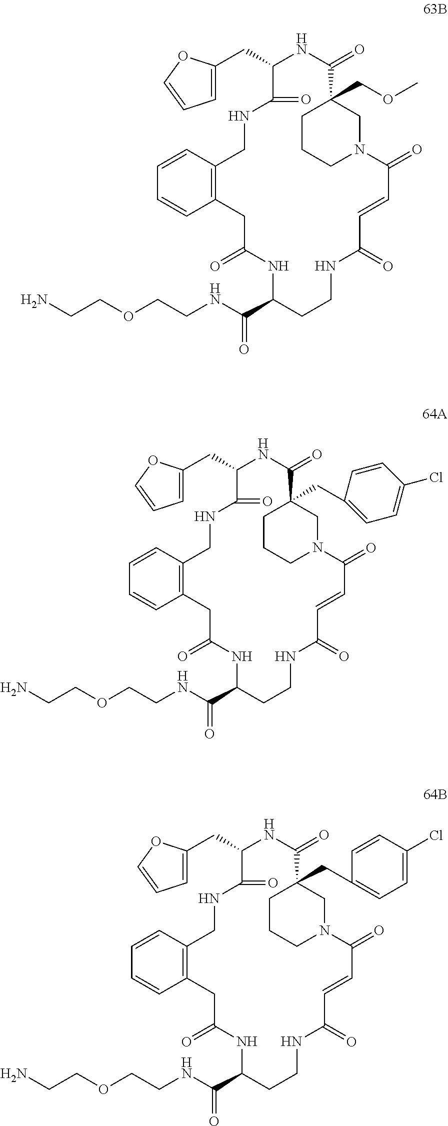 [SMILES: COC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; NCCOCCNC(=O)[C@@H]1CCNC(=O)/C=C/C(=O)N2CCC[C@](Cc3ccc(Cl)cc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(Cl)cc3)(C2)C(=O)N[C@@H](Cc2ccco2)C(=O)NCc2ccccc2CC(=O)N1]

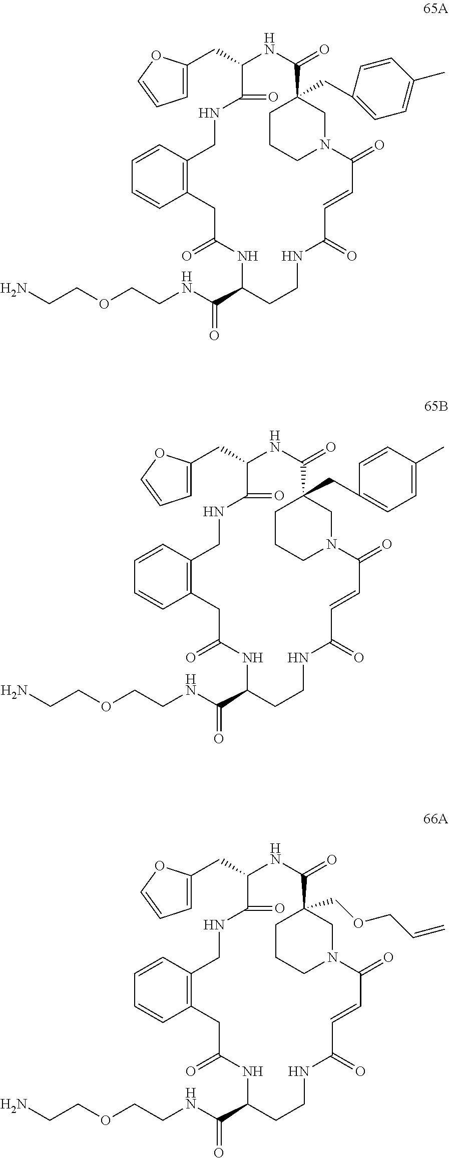 [SMILES: Cc1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; Cc1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; C=CCOC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

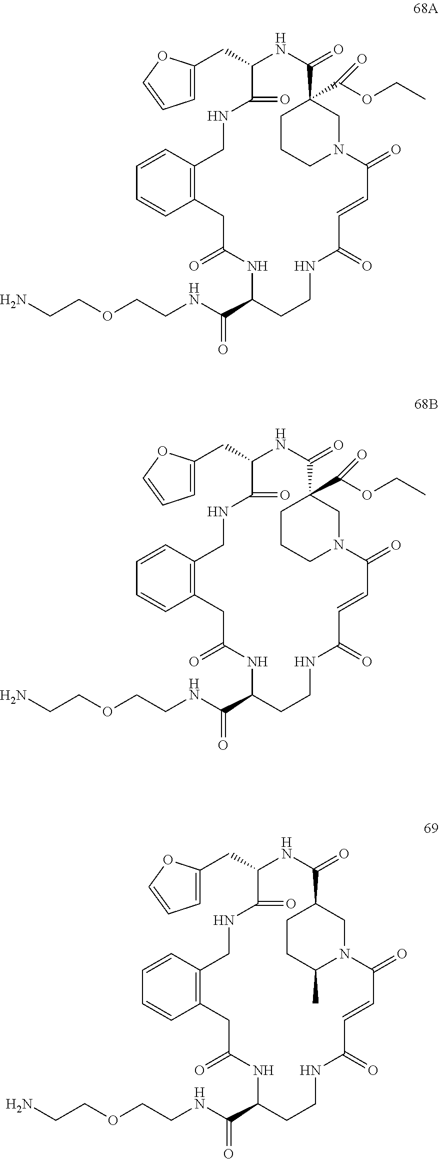 [SMILES: CCOC(=O)[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CCOC(=O)[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; C[C@H]1CC[C@@H]2CN1C(=O)/C=C/C(=O)NCC[C@@H](C(=O)NCCOCCN)NC(=O)Cc1ccccc1CNC(=O)[C@H](Cc1ccco1)NC2=O]

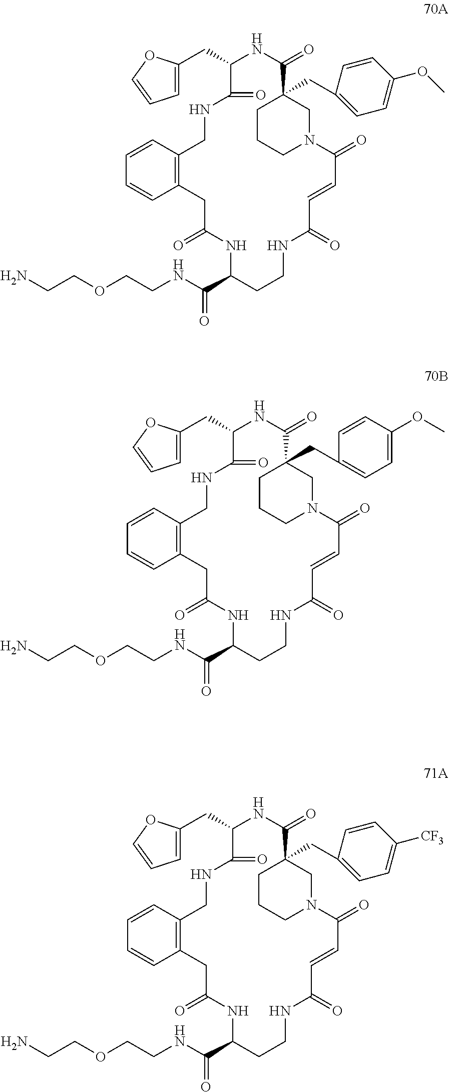 [SMILES: COc1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; COc1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; NCCOCCNC(=O)[C@@H]1CCNC(=O)/C=C/C(=O)N2CCC[C@](Cc3ccc(C(F)(F)F)cc3)(C2)C(=O)N[C@@H](Cc2ccco2)C(=O)NCc2ccccc2CC(=O)N1]

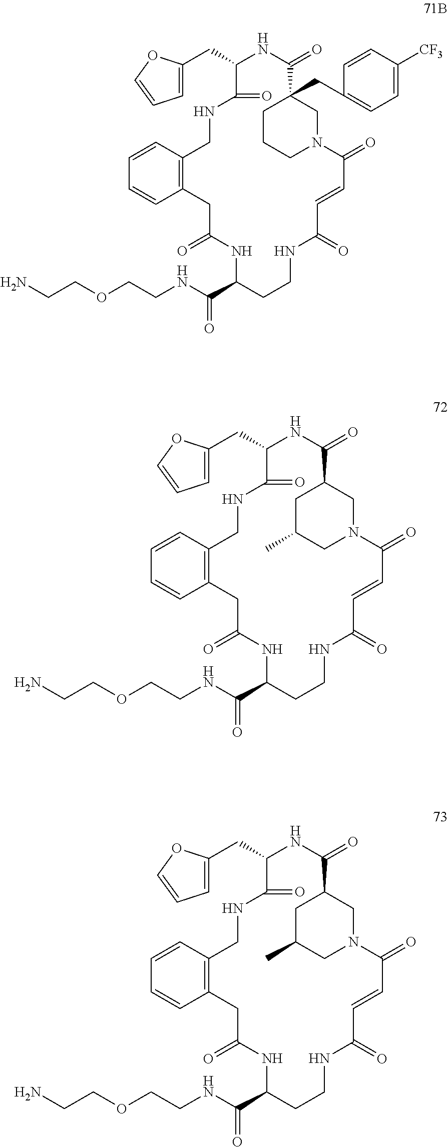 [SMILES: NCCOCCNC(=O)[C@@H]1CCNC(=O)/C=C/C(=O)N2CCC[C@@](Cc3ccc(C(F)(F)F)cc3)(C2)C(=O)N[C@@H](Cc2ccco2)C(=O)NCc2ccccc2CC(=O)N1 ; C[C@@H]1C[C@@H]2CN(C1)C(=O)/C=C/C(=O)NCC[C@@H](C(=O)NCCOCCN)NC(=O)Cc1ccccc1CNC(=O)[C@H](Cc1ccco1)NC2=O ; C[C@H]1C[C@@H]2CN(C1)C(=O)/C=C/C(=O)NCC[C@@H](C(=O)NCCOCCN)NC(=O)Cc1ccccc1CNC(=O)[C@H](Cc1ccco1)NC2=O]

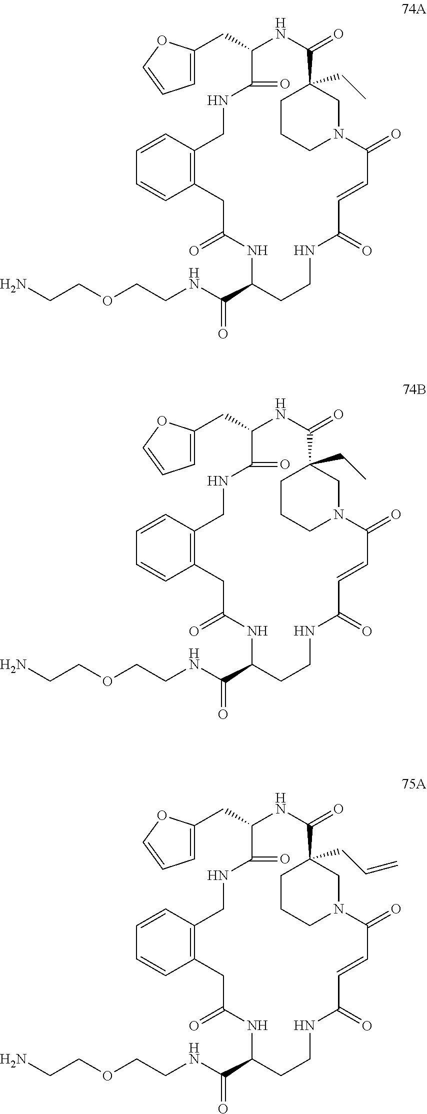 [SMILES: CC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; C=CC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O]

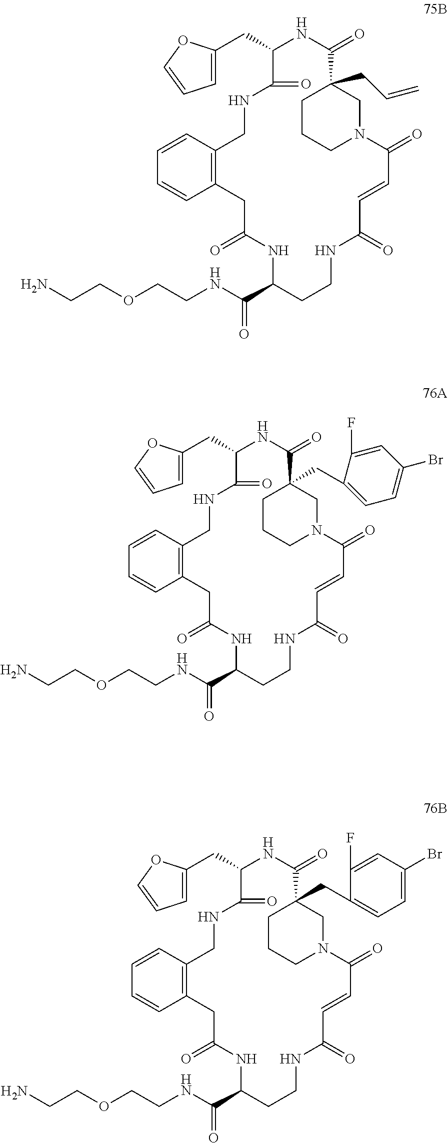 [SMILES: C=CC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; NCCOCCNC(=O)[C@@H]1CCNC(=O)/C=C/C(=O)N2CCC[C@](Cc3ccc(Br)cc3F)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(Br)cc3F)(C2)C(=O)N[C@@H](Cc2ccco2)C(=O)NCc2ccccc2CC(=O)N1]

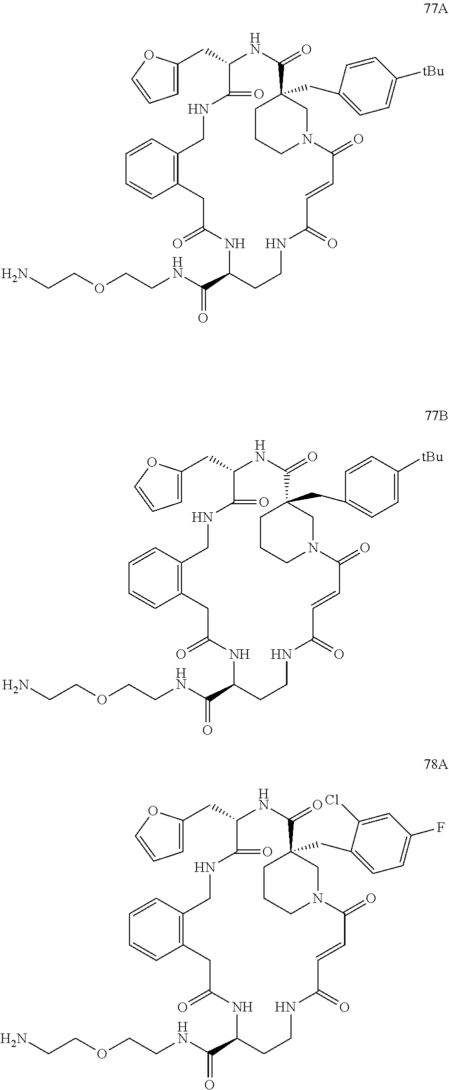 [SMILES: CC(C)(C)c1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; CC(C)(C)c1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; NCCOCCNC(=O)[C@@H]1CCNC(=O)/C=C/C(=O)N2CCC[C@](Cc3ccc(F)cc3Cl)(C2)C(=O)N[C@@H](Cc2ccco2)C(=O)NCc2ccccc2CC(=O)N1]

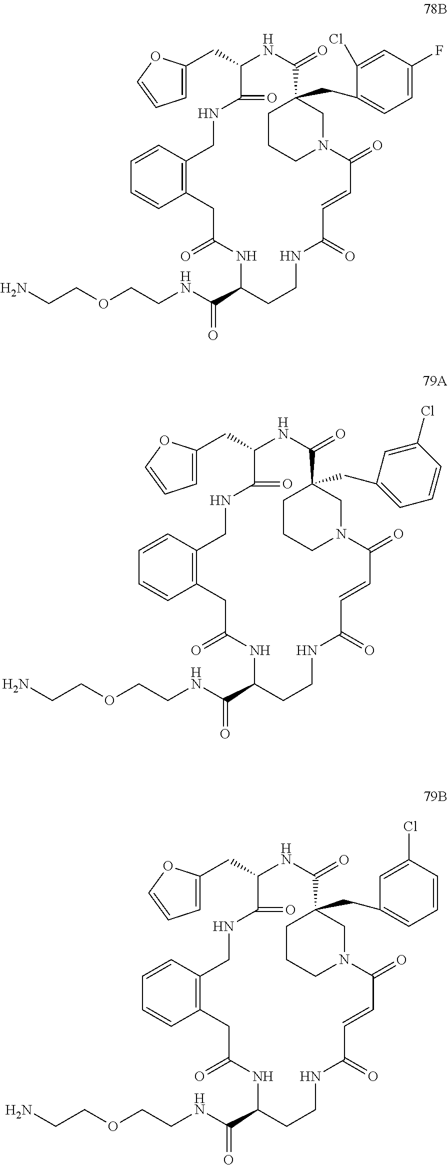 [SMILES: NCCOCCNC(=O)[C@@H]1CCNC(=O)/C=C/C(=O)N2CCC[C@@](Cc3ccc(F)cc3Cl)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3cccc(Cl)c3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3cccc(Cl)c3)(C2)C(=O)N[C@@H](Cc2ccco2)C(=O)NCc2ccccc2CC(=O)N1]

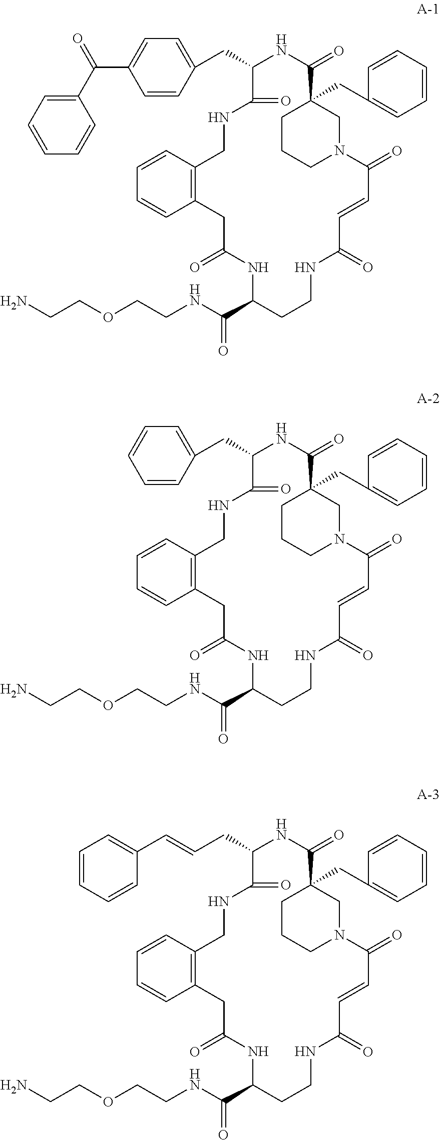 [SMILES: NCCOCCNC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](C/C=C/c2ccccc2)C(=O)NCc2ccccc2CC(=O)N1]

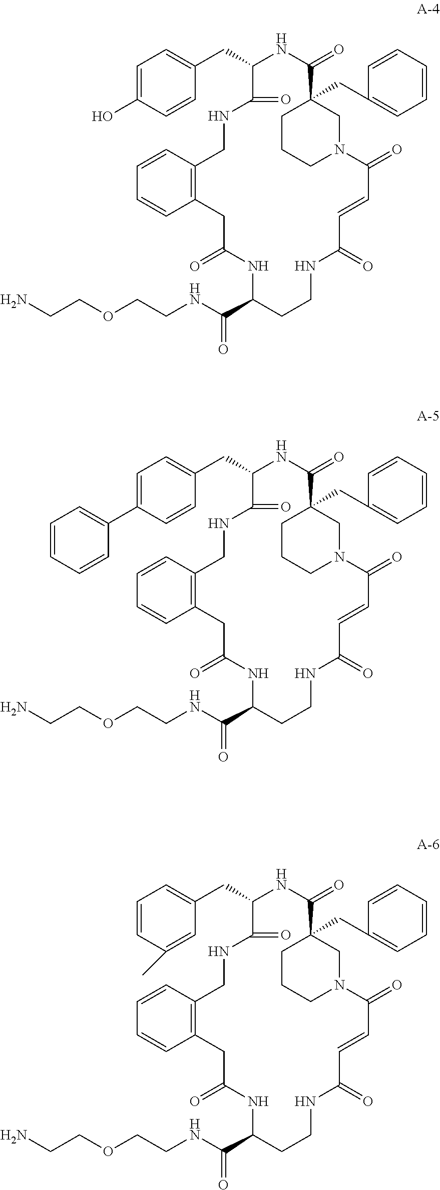 [SMILES: NCCOCCNC(=O)[C@@H]1CCNC(=O)/C=C/C(=O)N2CCC[C@](Cc3ccccc3)(C2)C(=O)N[C@@H](Cc2ccc(O)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccccc3)cc2)C(=O)NCc2ccccc2CC(=O)N1 ; Cc1cccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)c1]

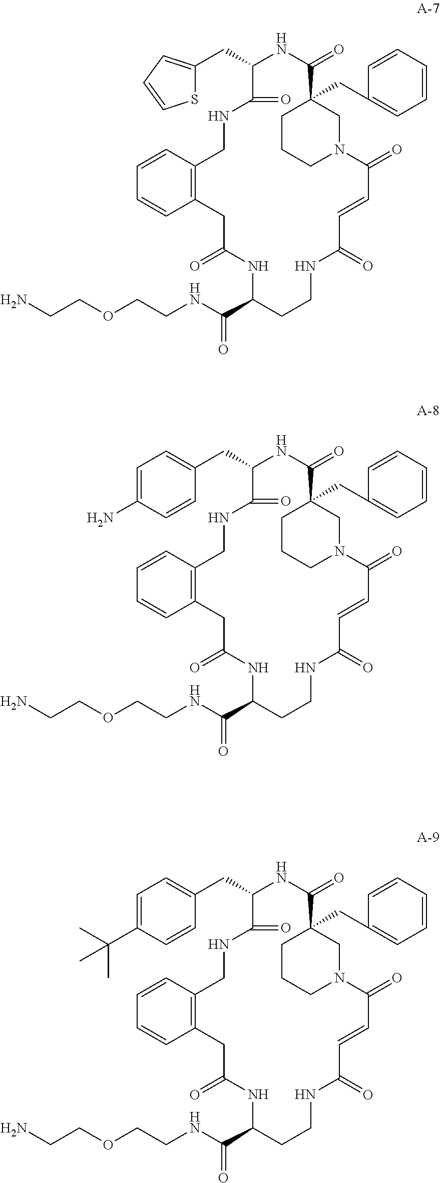 [SMILES: NCCOCCNC(=O)[C@@H]1CCNC(=O)/C=C/C(=O)N2CCC[C@](Cc3ccccc3)(C2)C(=O)N[C@@H](Cc2cccs2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(N)cc2)C(=O)NCc2ccccc2CC(=O)N1 ; CC(C)(C)c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1]

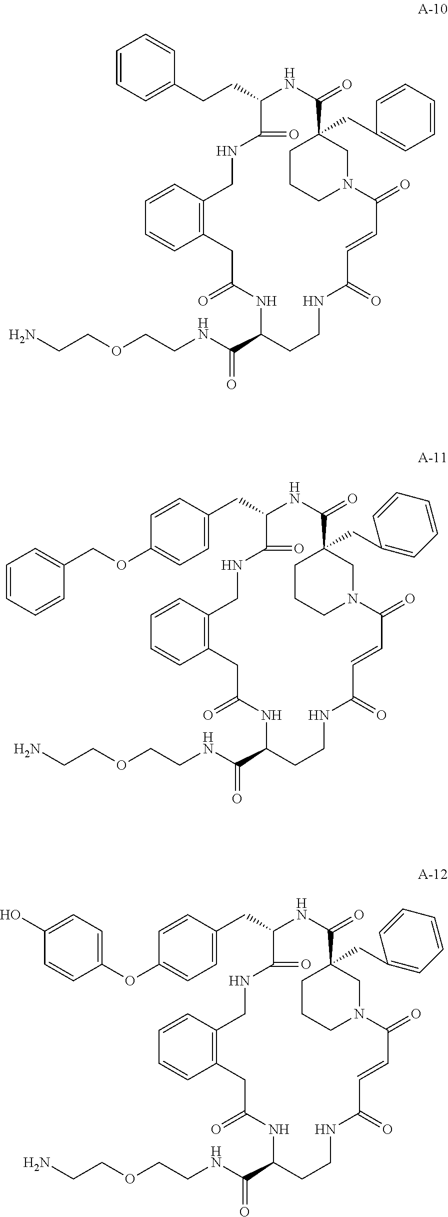 [SMILES: NCCOCCNC(=O)[C@@H]1CCNC(=O)/C=C/C(=O)N2CCC[C@](Cc3ccccc3)(C2)C(=O)N[C@@H](CCc2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(OCc3ccccc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(Oc3ccc(O)cc3)cc2)C(=O)NCc2ccccc2CC(=O)N1]

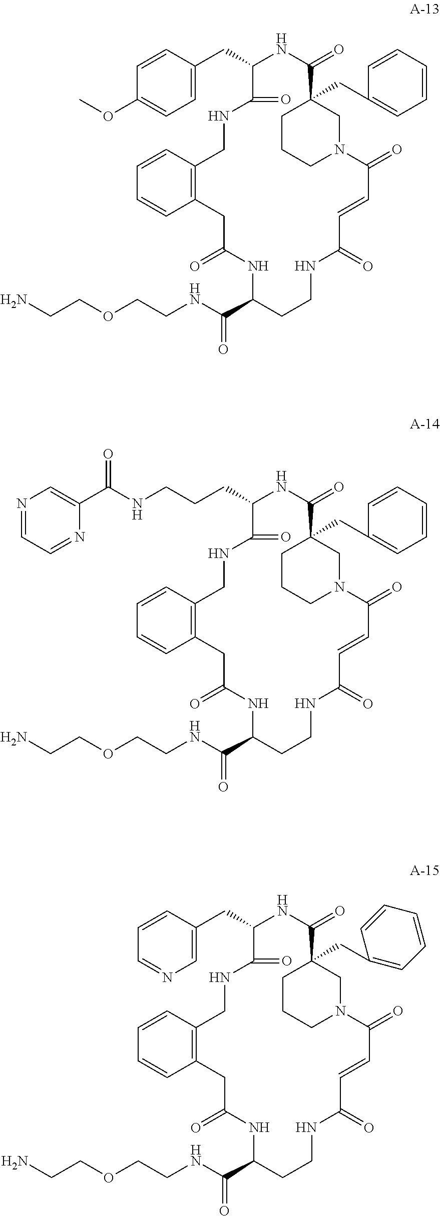 [SMILES: COc1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](CCCNC(=O)c2cnccn2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccnc2)C(=O)NCc2ccccc2CC(=O)N1]

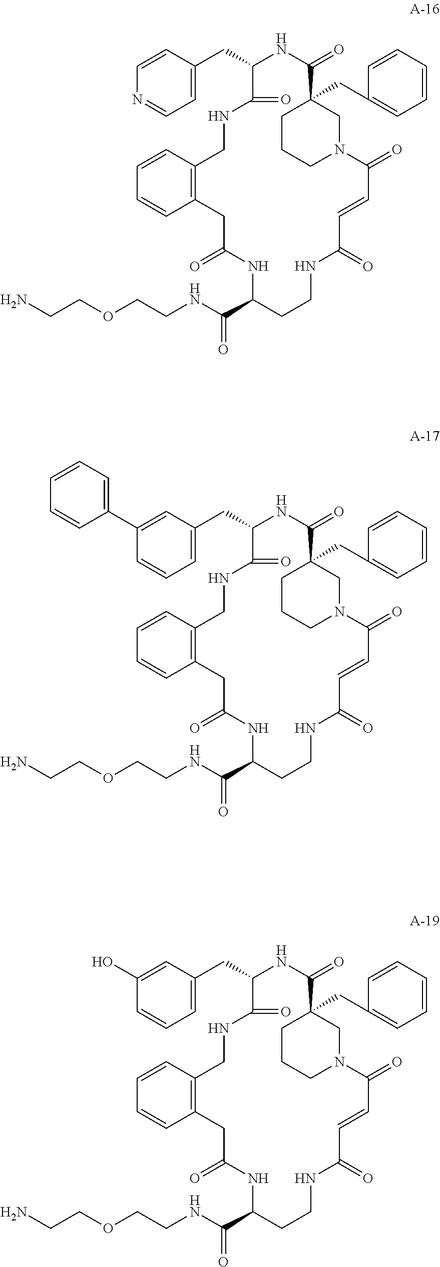 [SMILES: NCCOCCNC(=O)[C@@H]1CCNC(=O)/C=C/C(=O)N2CCC[C@](Cc3ccccc3)(C2)C(=O)N[C@@H](Cc2ccncc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccc(-c3ccccc3)c2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccc(O)c2)C(=O)NCc2ccccc2CC(=O)N1]

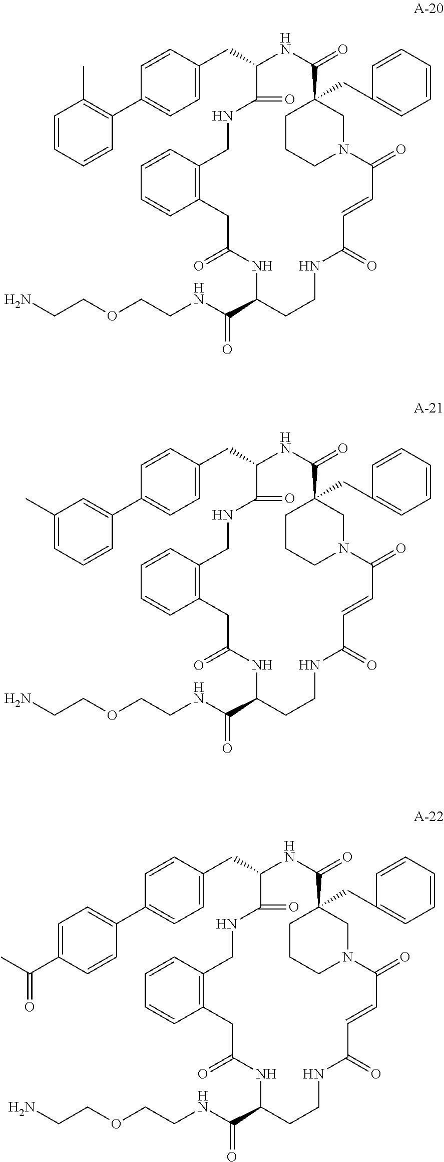 [SMILES: Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; Cc1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

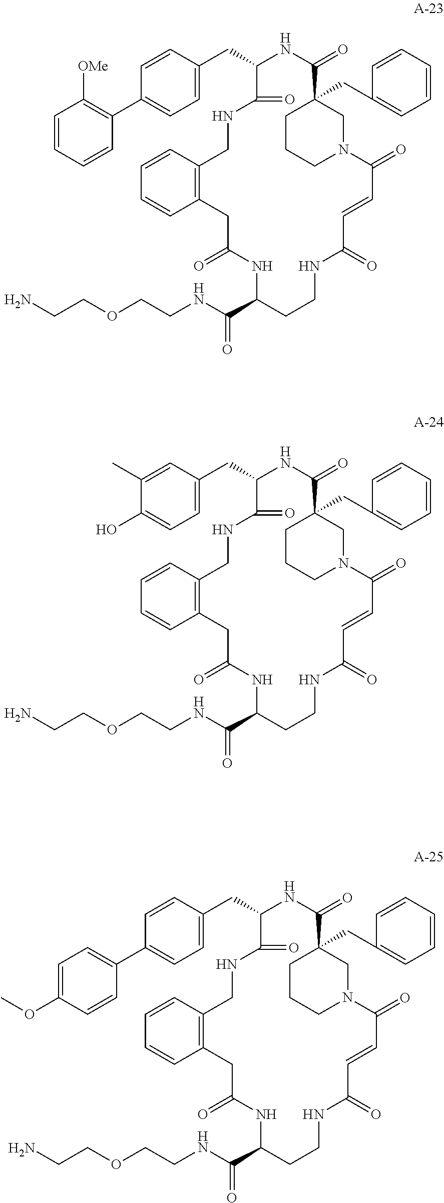 [SMILES: COc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; Cc1cc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)ccc1O ; COc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

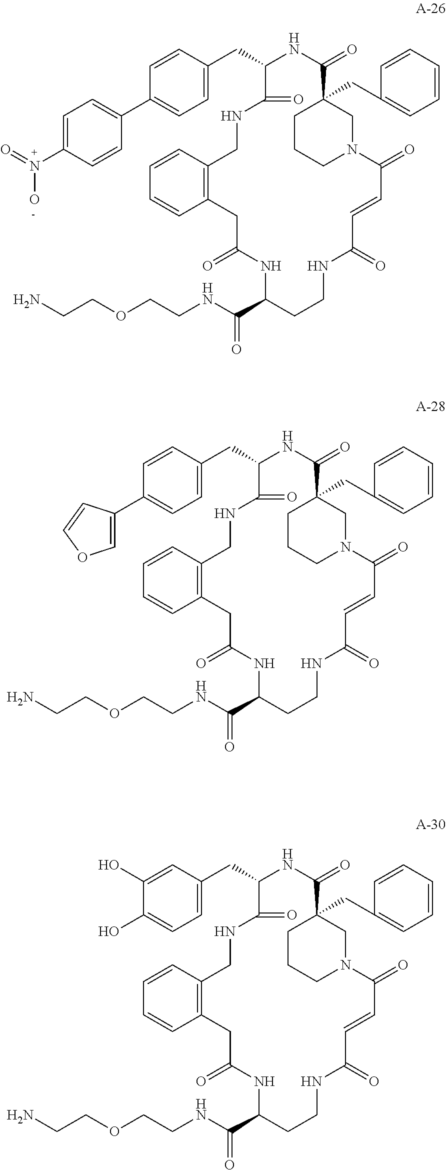

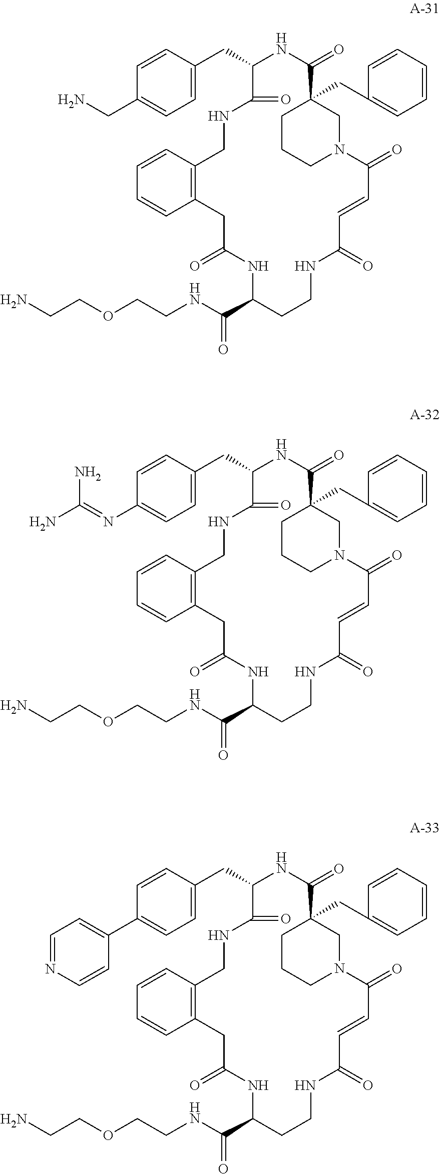 [SMILES: NCCOCCNC(=O)[C@@H]1CCNC(=O)/C=C/C(=O)N2CCC[C@](Cc3ccccc3)(C2)C(=O)N[C@@H](Cc2ccc(CN)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(N=C(N)N)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccncc3)cc2)C(=O)NCc2ccccc2CC(=O)N1]

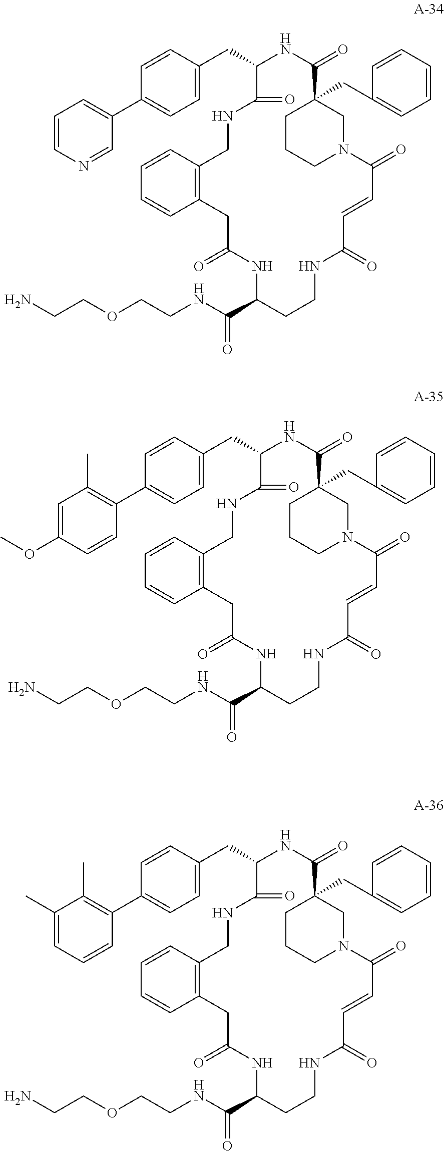 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3cccnc3)cc2)C(=O)NCc2ccccc2CC(=O)N1 ; COc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(C)c1 ; Cc1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1C]

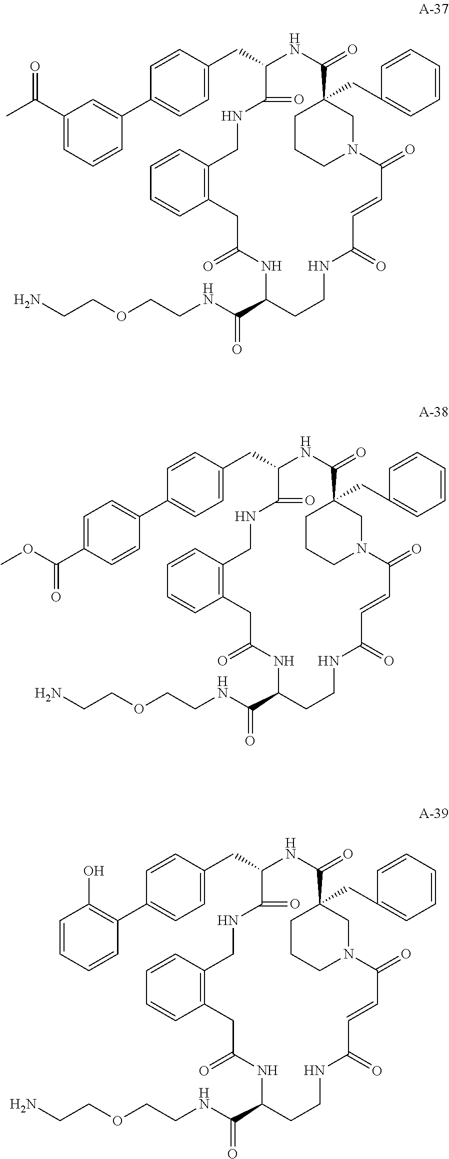 [SMILES: CC(=O)c1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1 ; COC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccccc3O)cc2)C(=O)NCc2ccccc2CC(=O)N1]

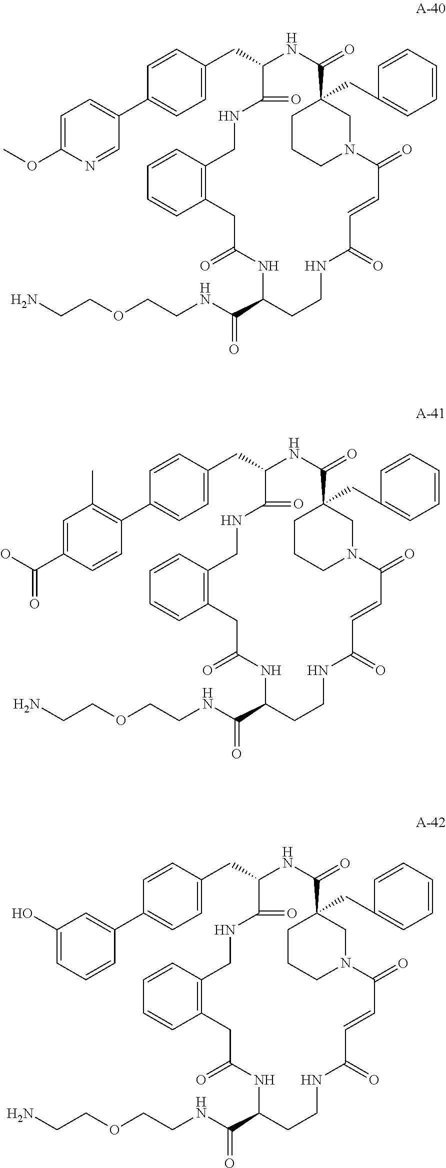 [SMILES: COc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cn1 ; Cc1cc(C(=O)O)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3cccc(O)c3)cc2)C(=O)NCc2ccccc2CC(=O)N1]

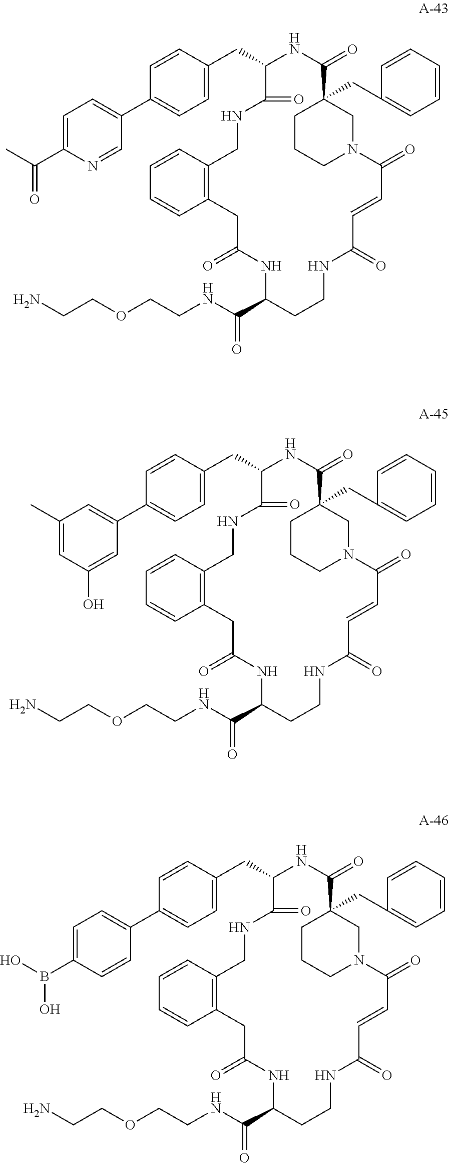 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cn1 ; Cc1cc(O)cc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(B(O)O)cc3)cc2)C(=O)NCc2ccccc2CC(=O)N1]

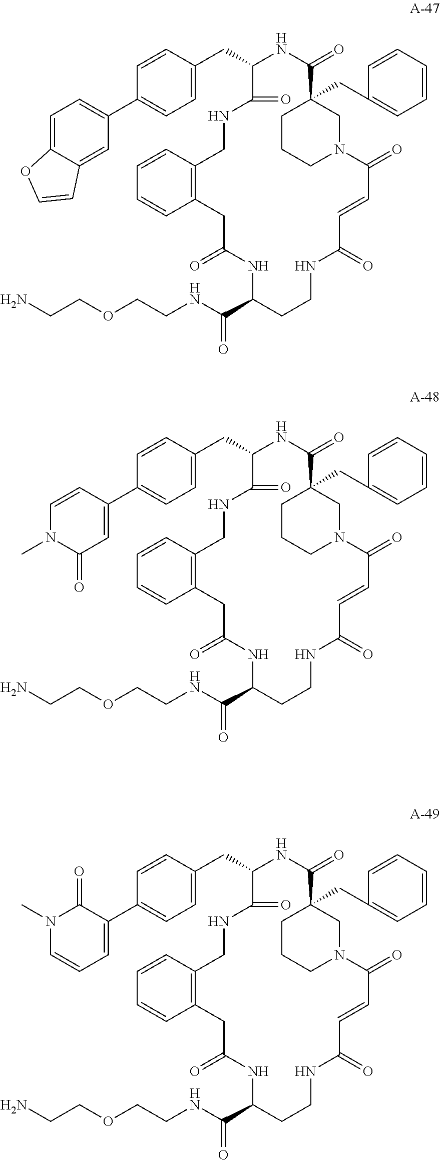 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc4occc4c3)cc2)C(=O)NCc2ccccc2CC(=O)N1 ; Cn1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1=O ; Cn1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1=O]

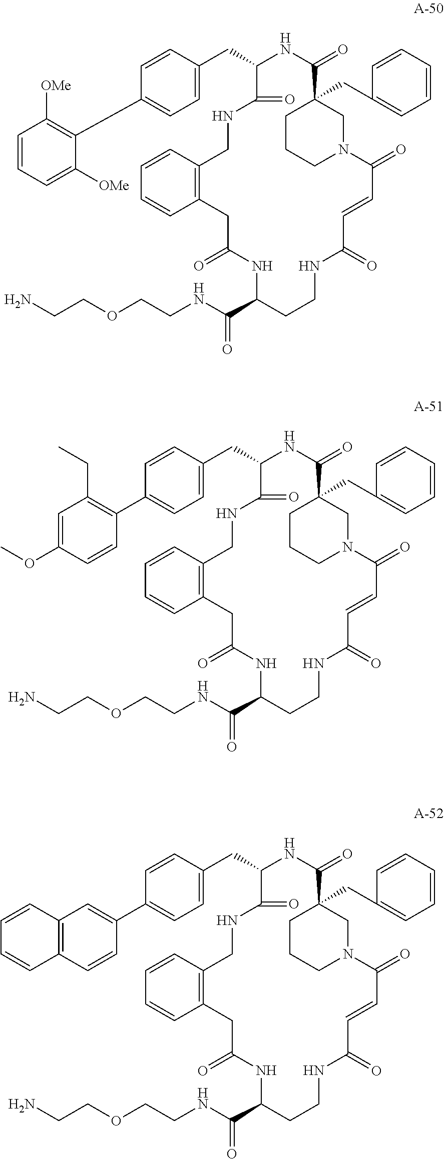 [SMILES: COc1cccc(OC)c1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; CCc1cc(OC)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc4ccccc4c3)cc2)C(=O)NCc2ccccc2CC(=O)N1]

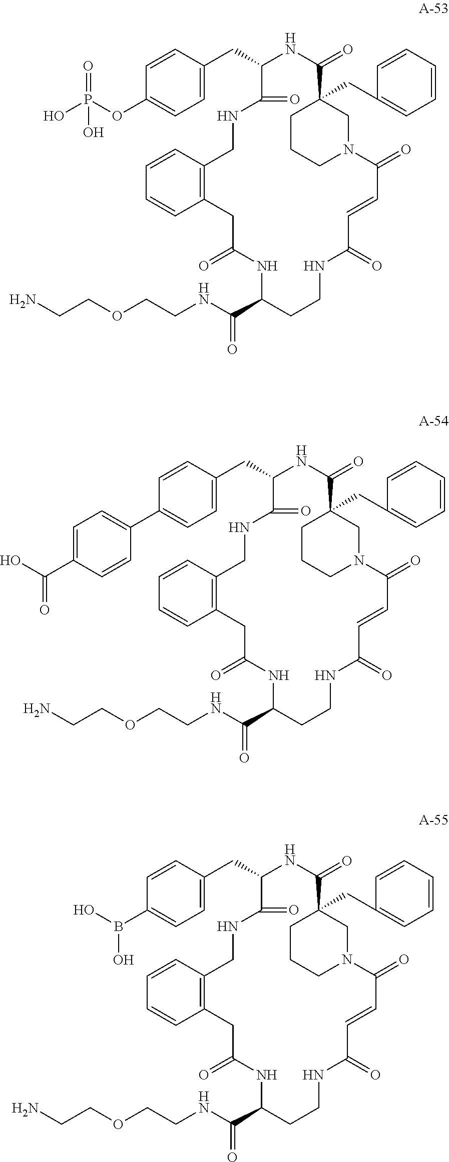 [SMILES: NCCOCCNC(=O)[C@@H]1CCNC(=O)/C=C/C(=O)N2CCC[C@](Cc3ccccc3)(C2)C(=O)N[C@@H](Cc2ccc(OP(=O)(O)O)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(C(=O)O)cc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(B(O)O)cc2)C(=O)NCc2ccccc2CC(=O)N1]

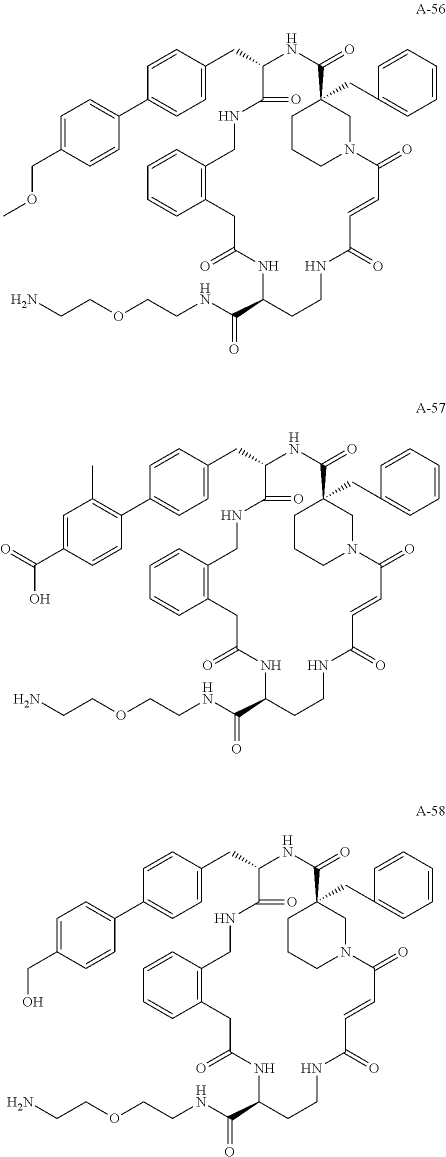 [SMILES: COCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; Cc1cc(C(=O)O)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CO)cc3)cc2)C(=O)NCc2ccccc2CC(=O)N1]

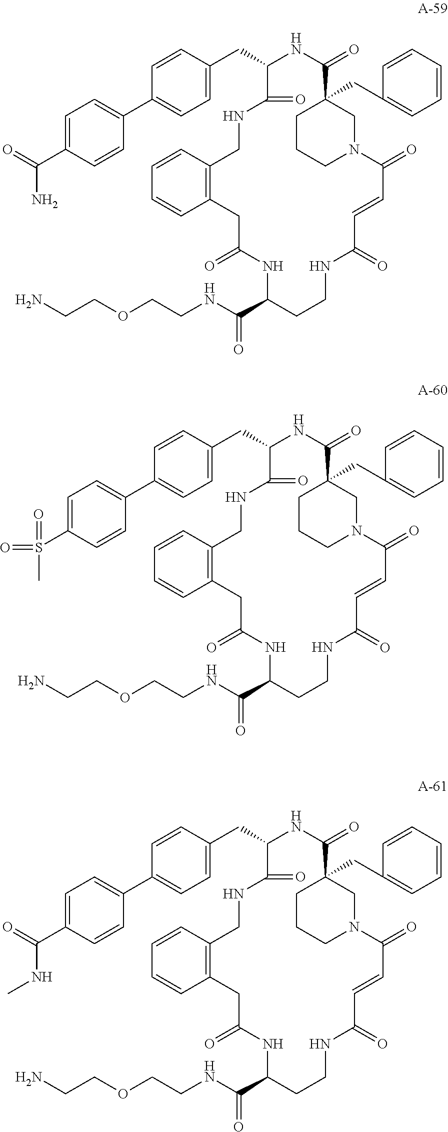 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc(C(N)=O)cc3)cc2)C(=O)NCc2ccccc2CC(=O)N1 ; CS(=O)(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CNC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

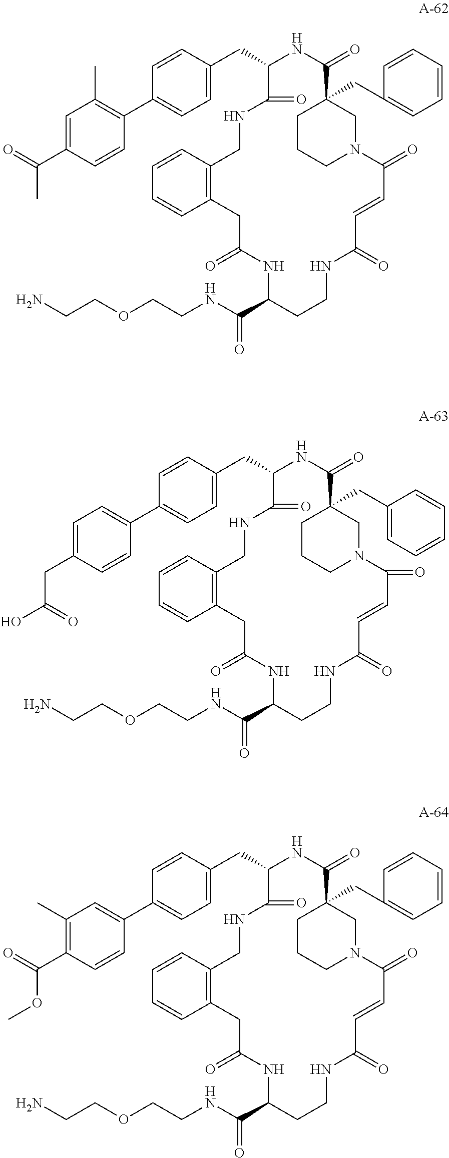 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(C)c1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CC(=O)O)cc3)cc2)C(=O)NCc2ccccc2CC(=O)N1 ; COC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1C]

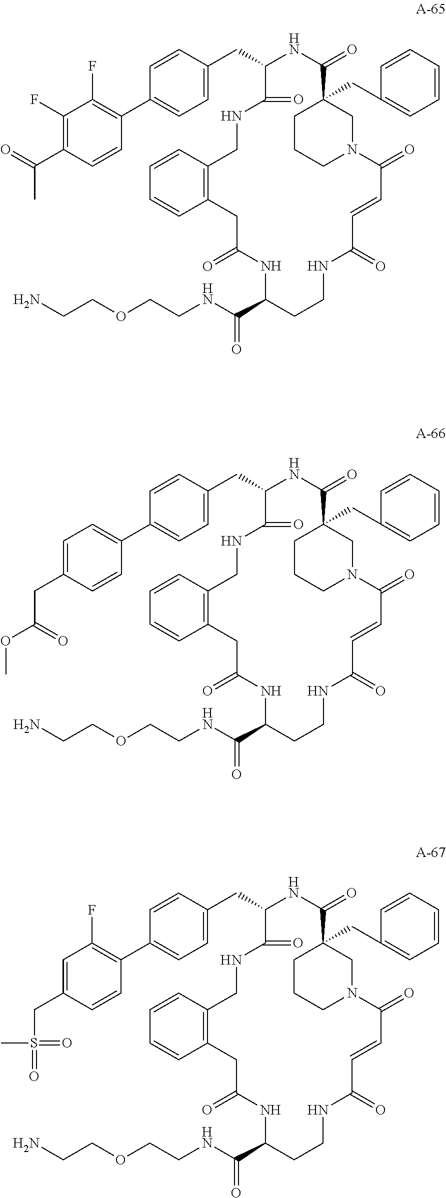 [SMILES: CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(F)c1F ; COC(=O)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CS(=O)(=O)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(F)c1]

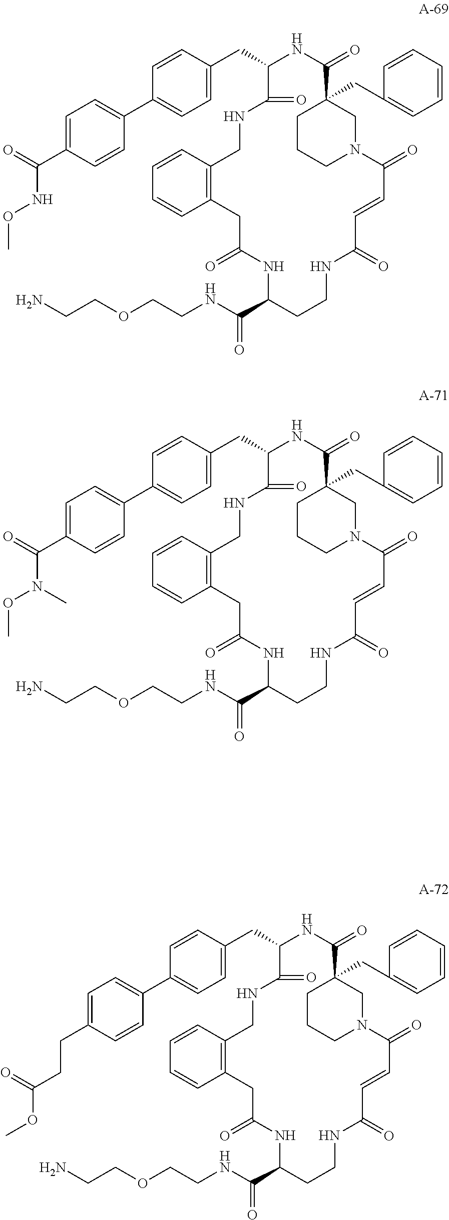 [SMILES: CONC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CON(C)C(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; COC(=O)CCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

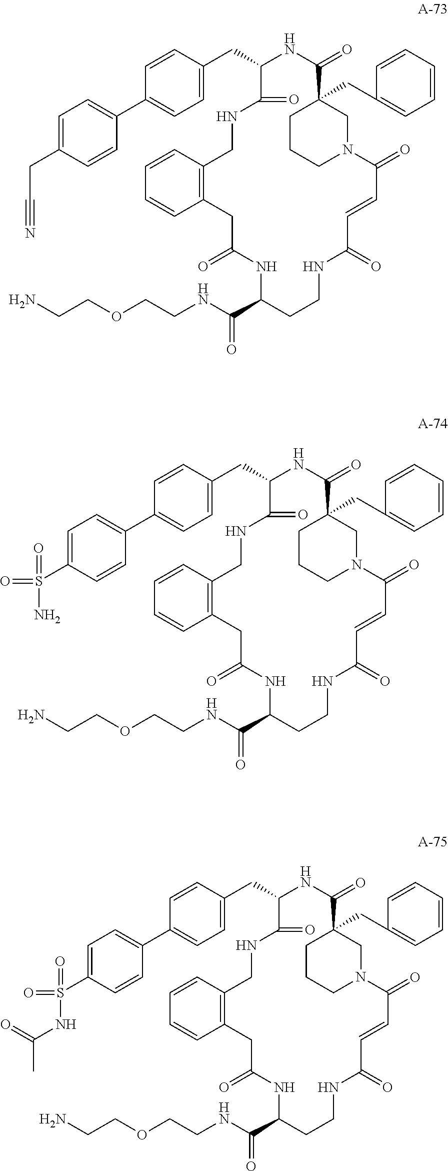 [SMILES: N#CCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(S(N)(=O)=O)cc3)cc2)C(=O)NCc2ccccc2CC(=O)N1 ; CC(=O)NS(=O)(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

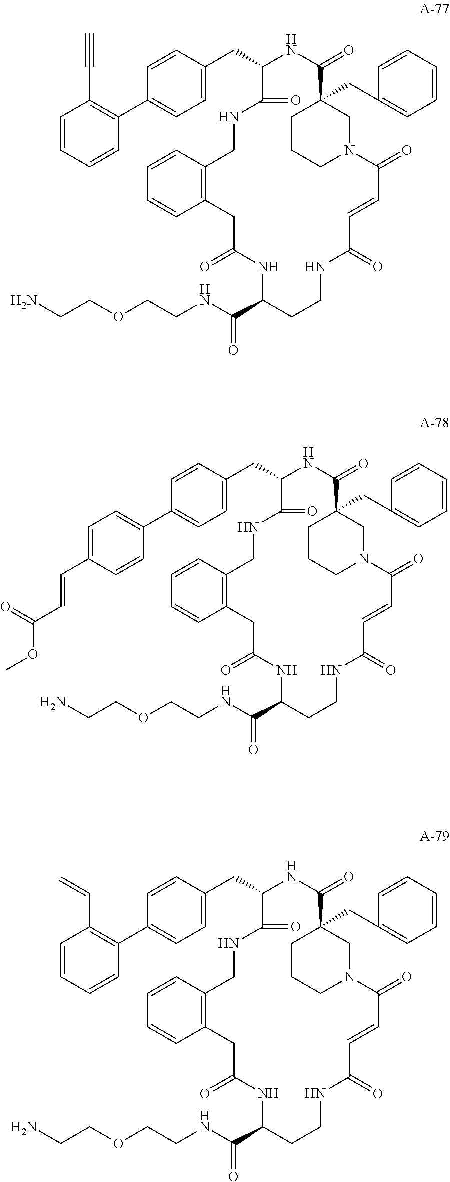 [SMILES: C#Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; COC(=O)/C=C/c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; C=Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1]

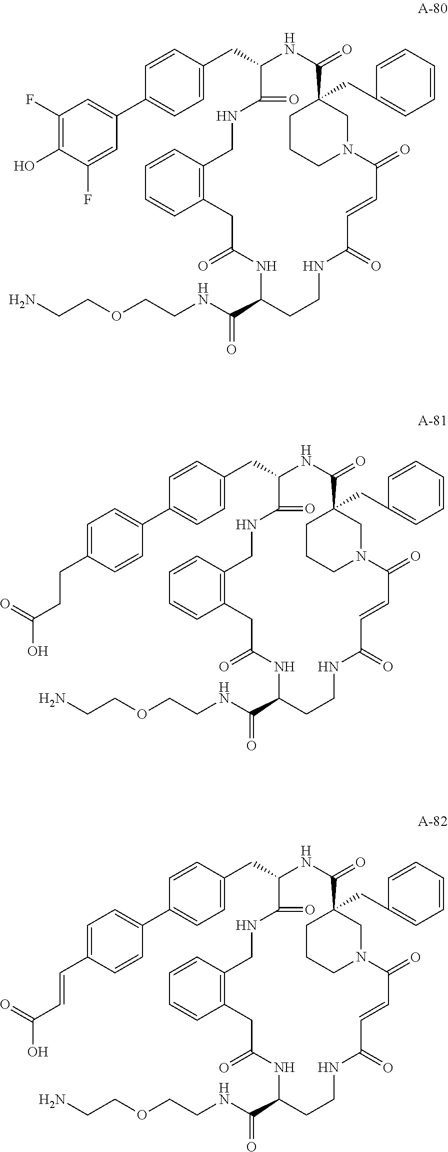 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3cc(F)c(O)c(F)c3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCC(=O)O)cc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(/C=C/C(=O)O)cc3)cc2)C(=O)NCc2ccccc2CC(=O)N1]

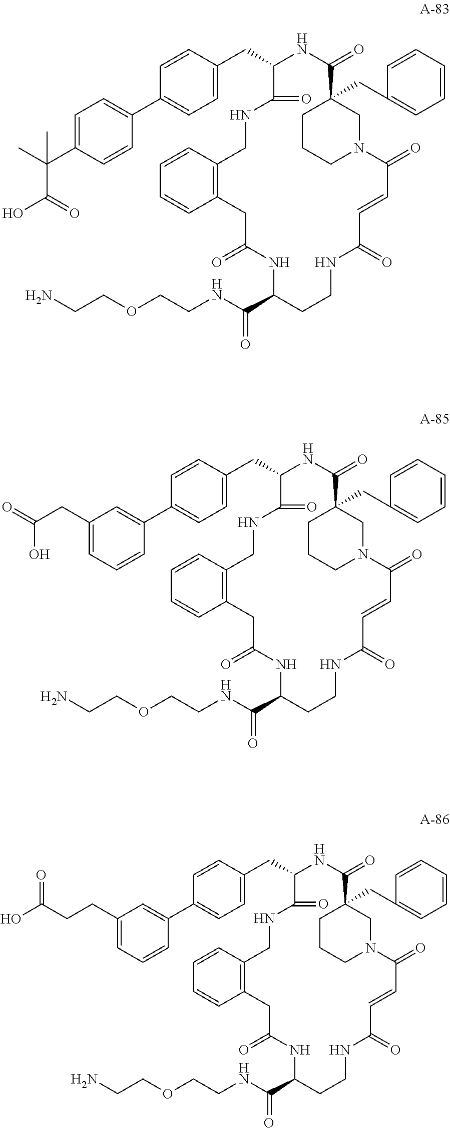 [SMILES: CC(C)(C(=O)O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3cccc(CC(=O)O)c3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3cccc(CCC(=O)O)c3)cc2)C(=O)NCc2ccccc2CC(=O)N1]

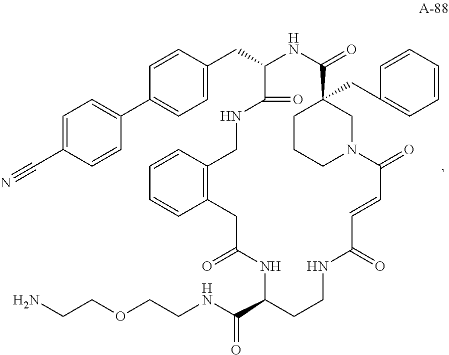 [SMILES: N#Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

or a pharmaceutically acceptable salt, solvate, hydrate, stereoisomer, polymorph, tautomer, isotopically enriched form, or prodrug thereof.

In certain embodiments, the compound of Formula (I′) or (I) is of the formula:

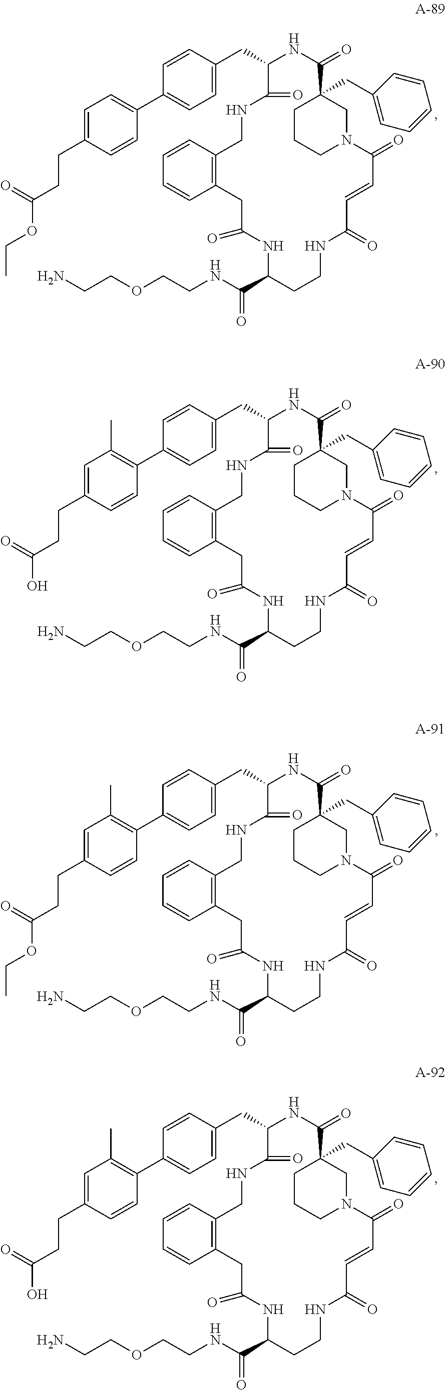

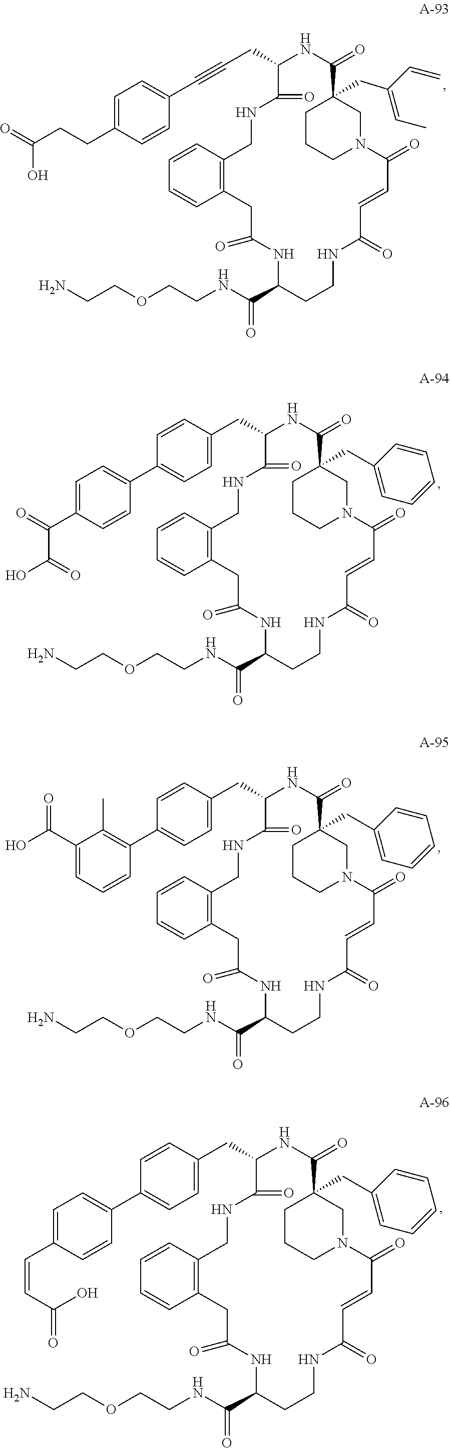

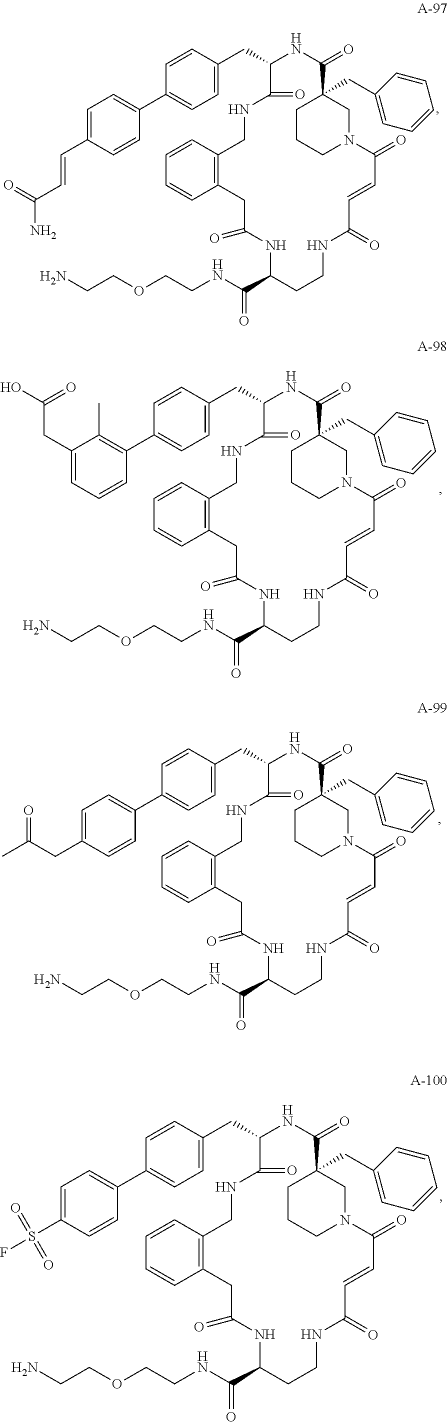

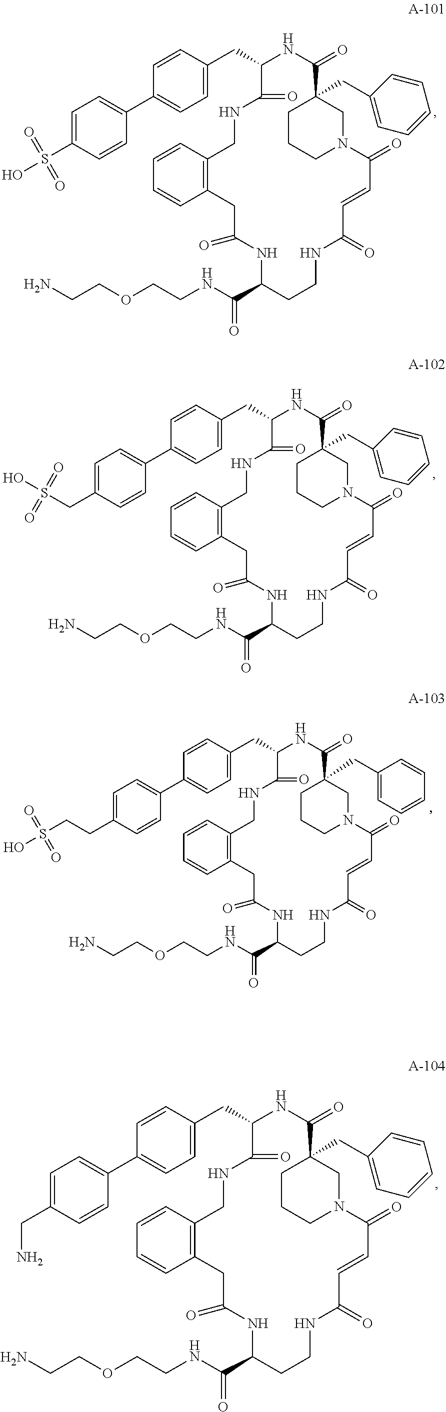

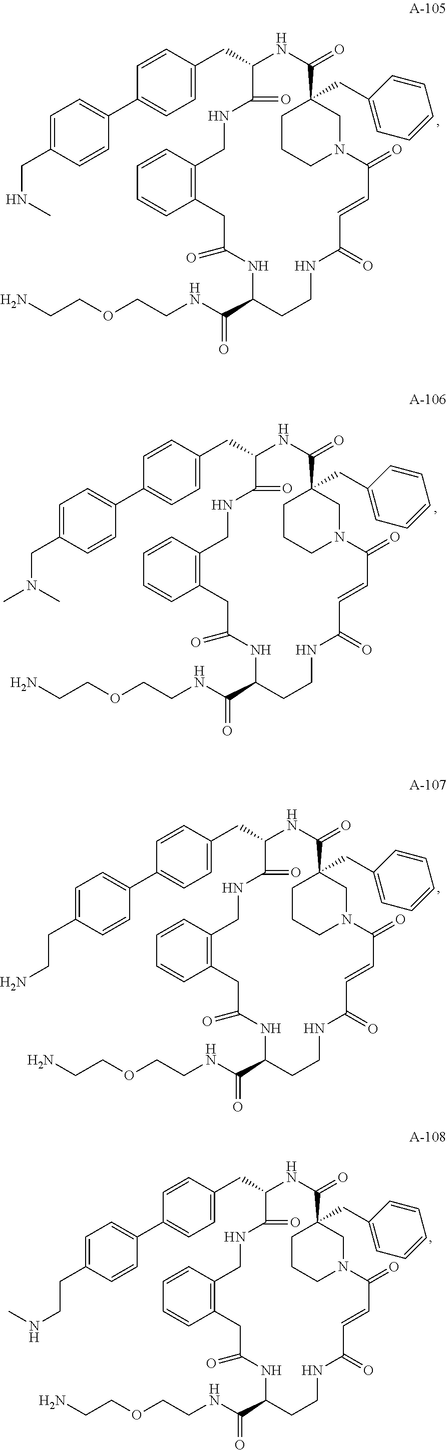 [SMILES: CNCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CN(C)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCN)cc3)cc2)C(=O)NCc2ccccc2CC(=O)N1 ; CNCCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]

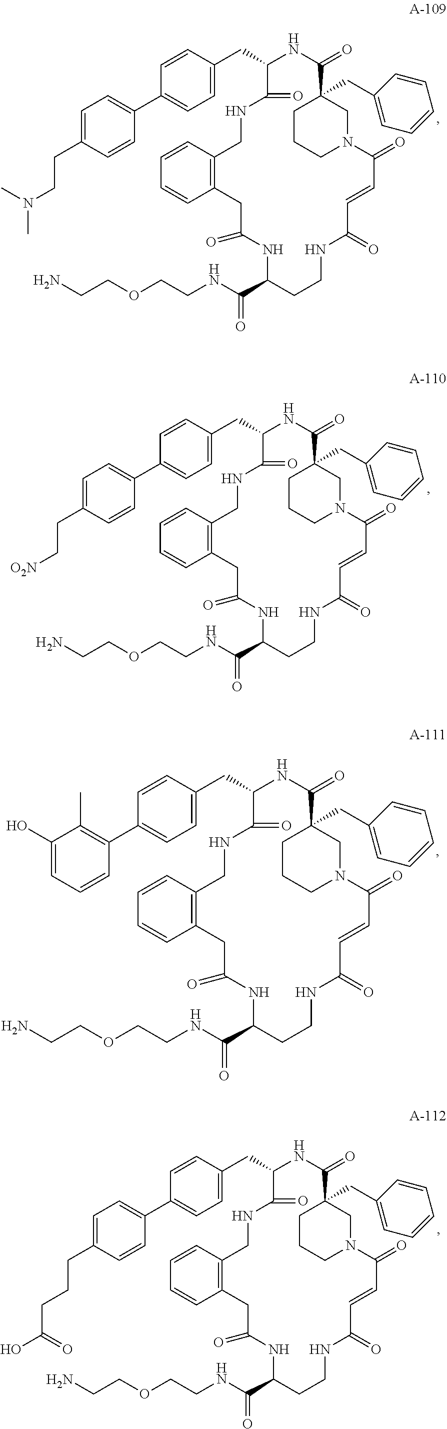

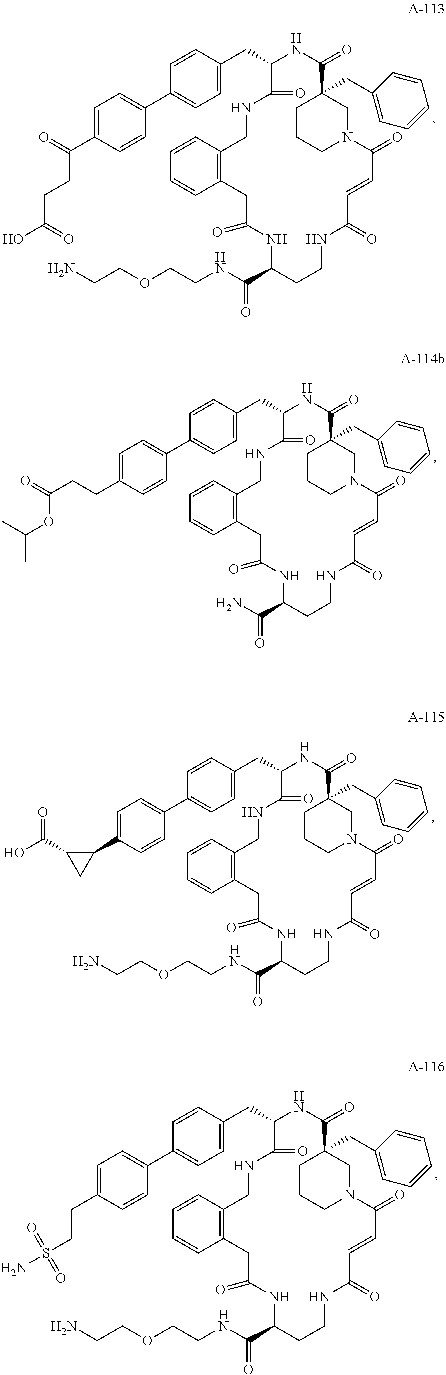

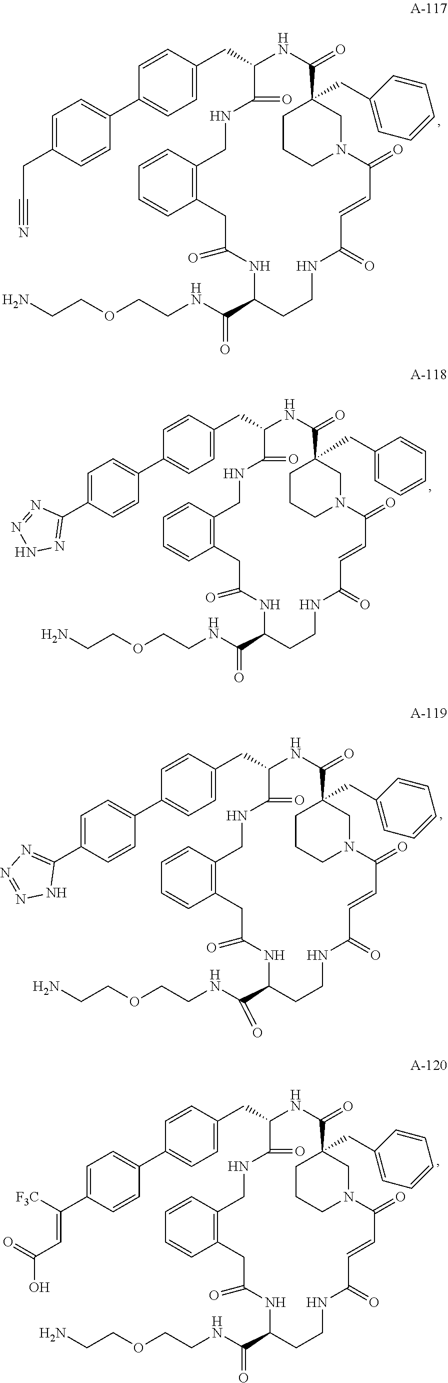

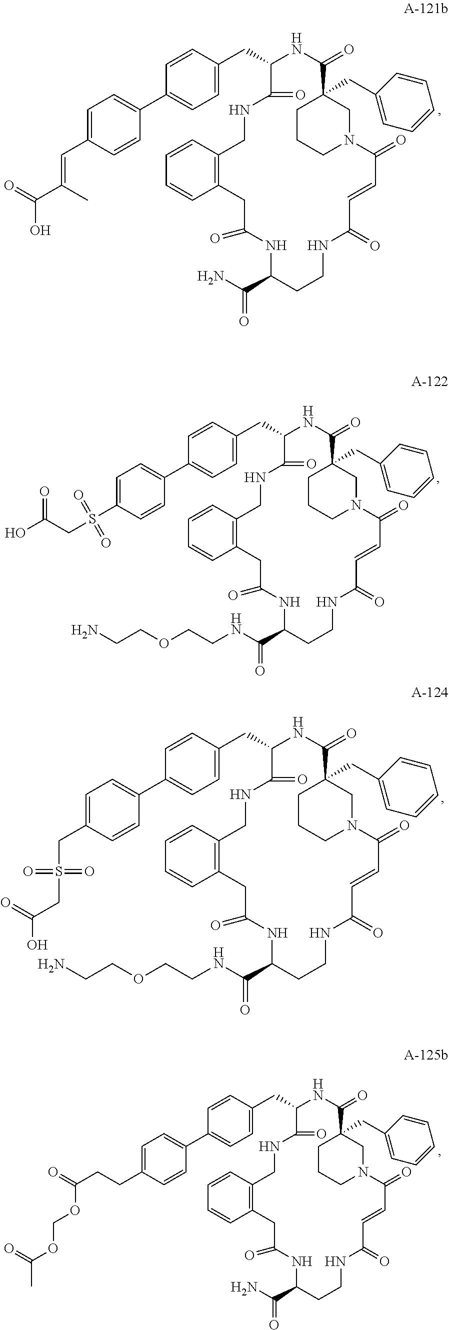

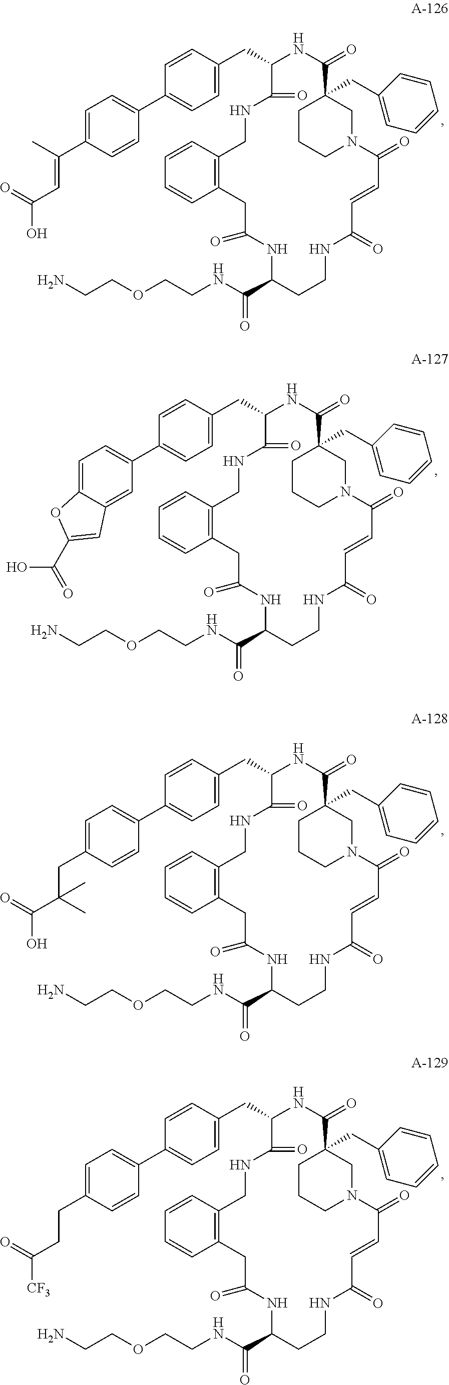

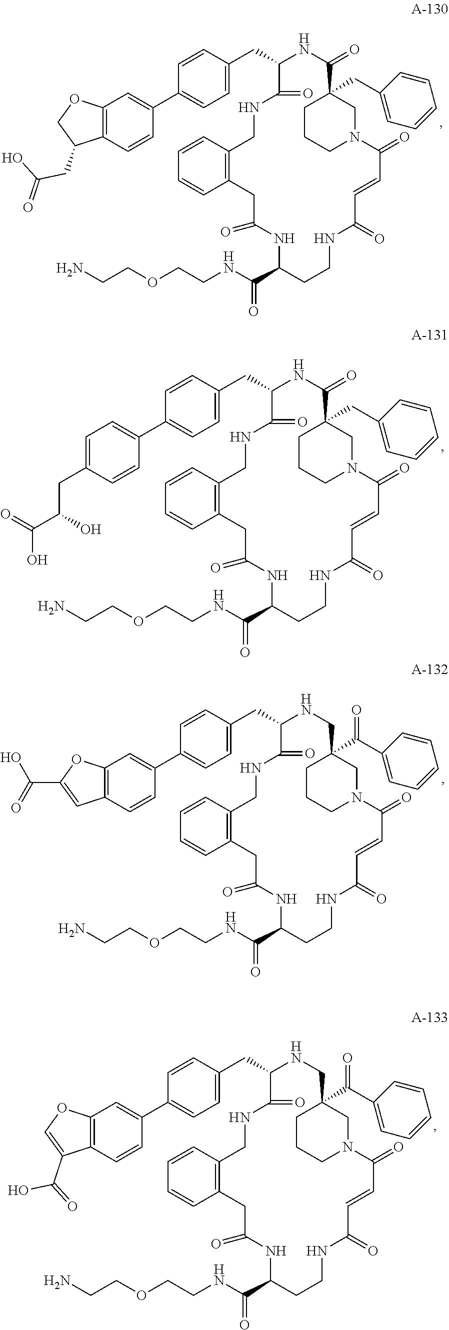

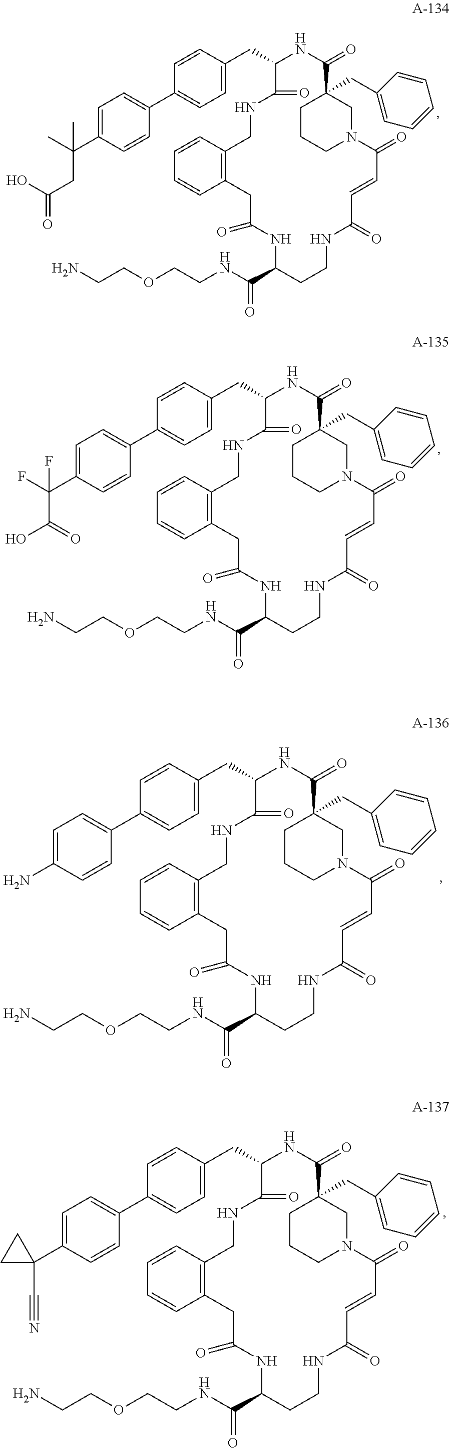

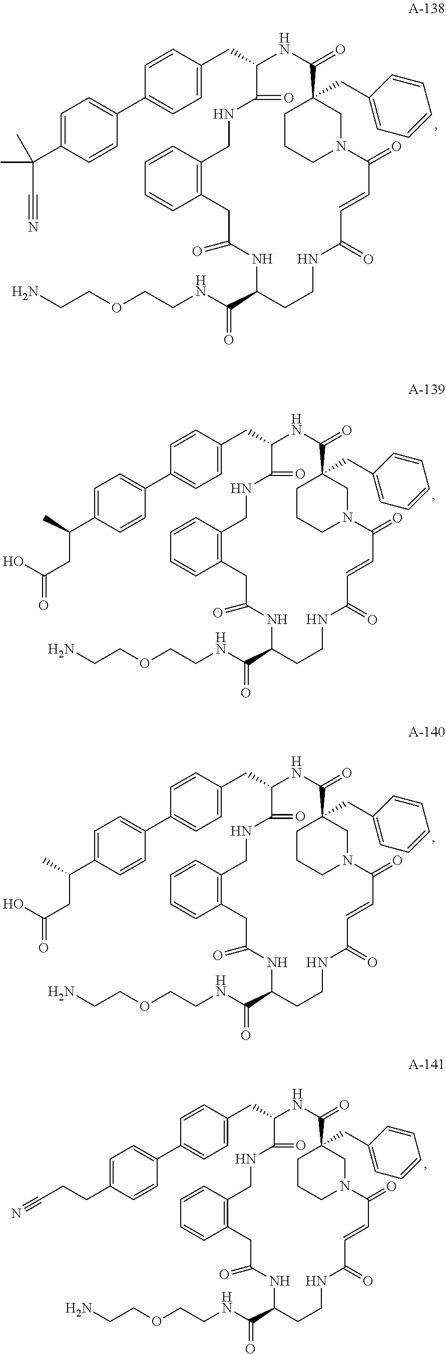

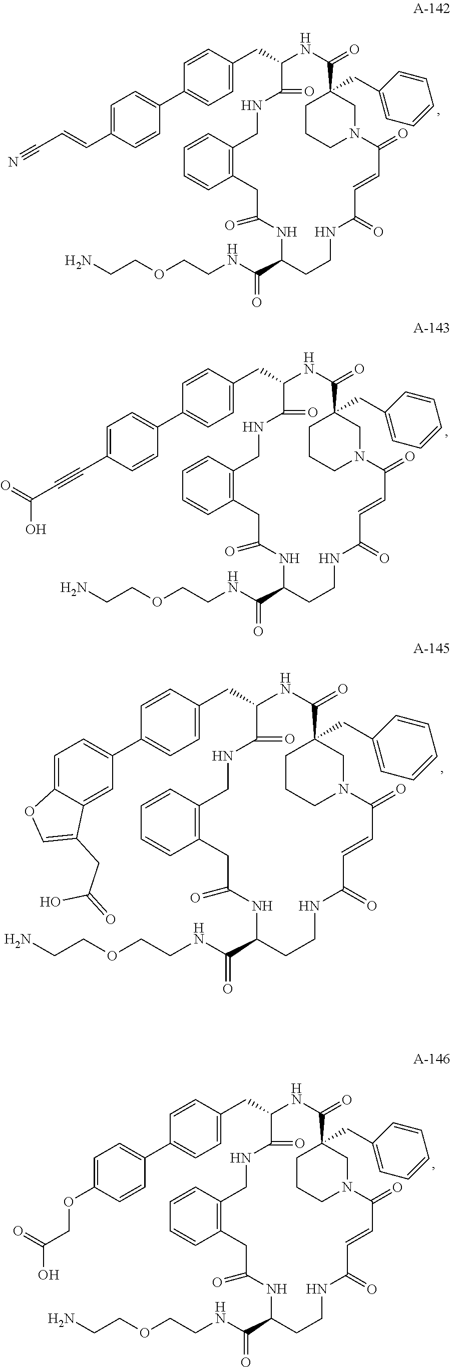

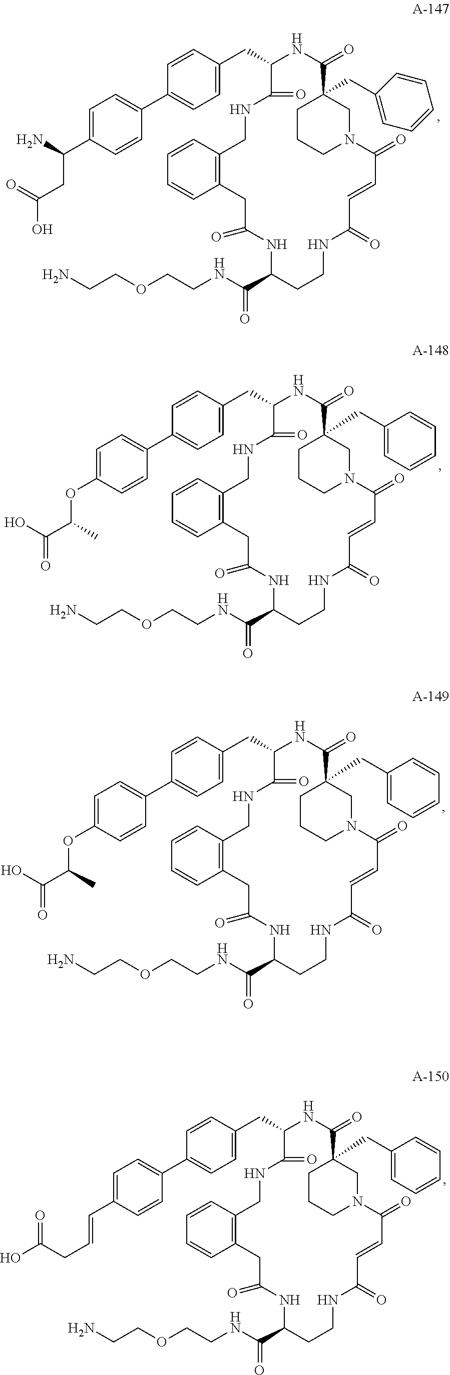

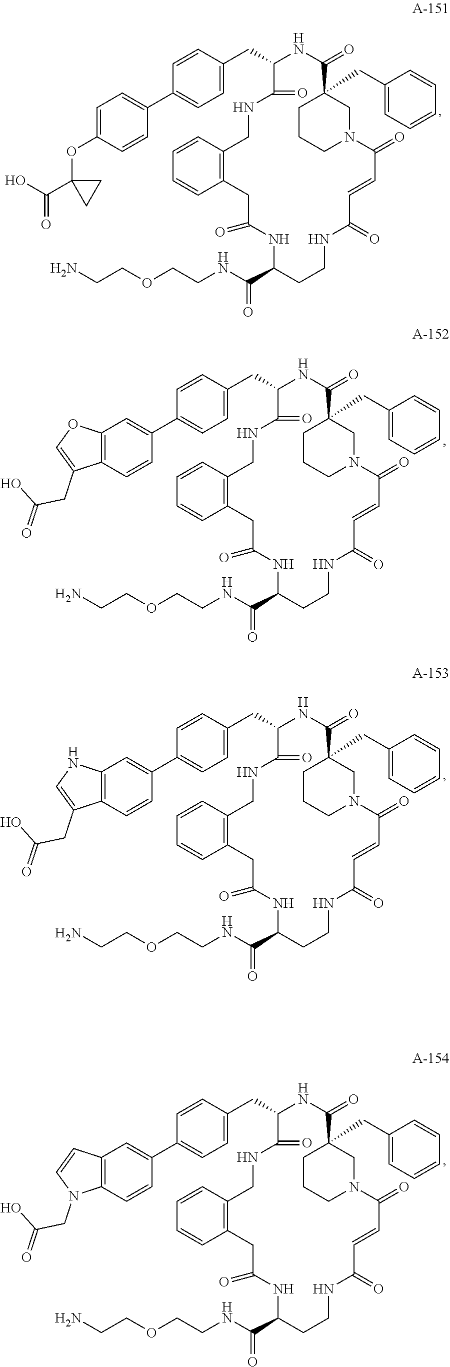

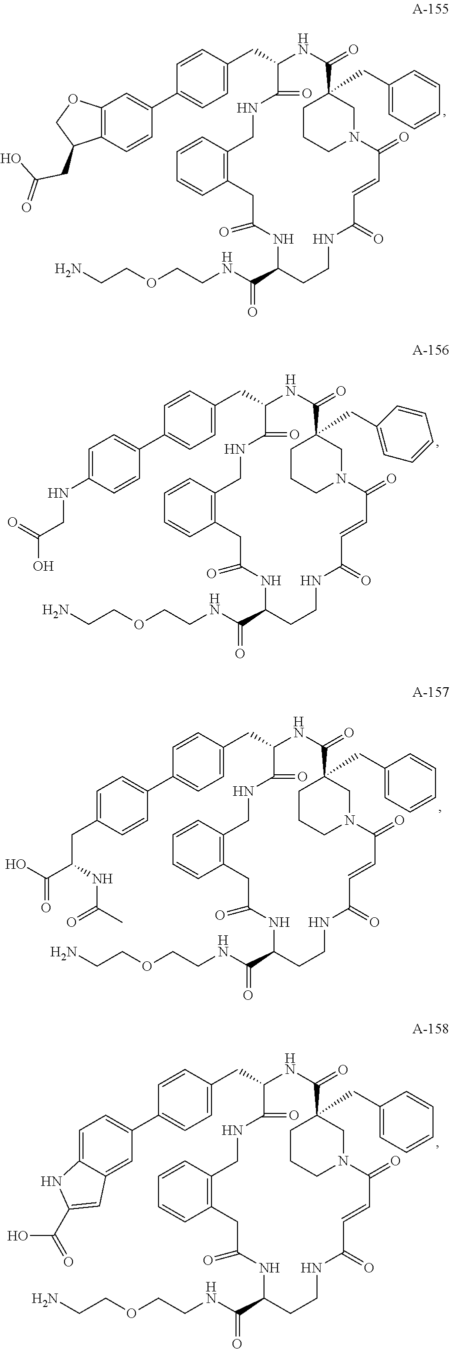

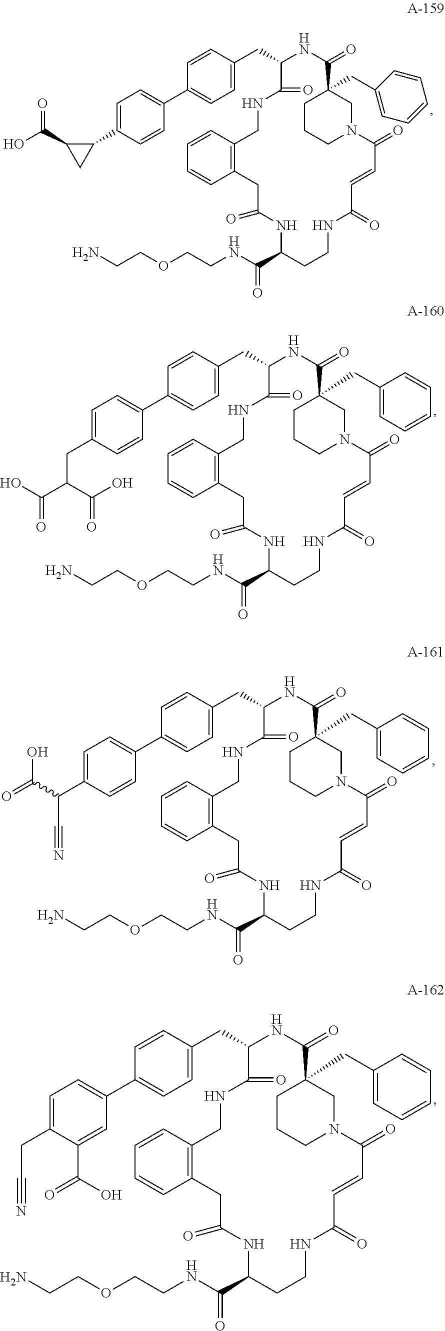

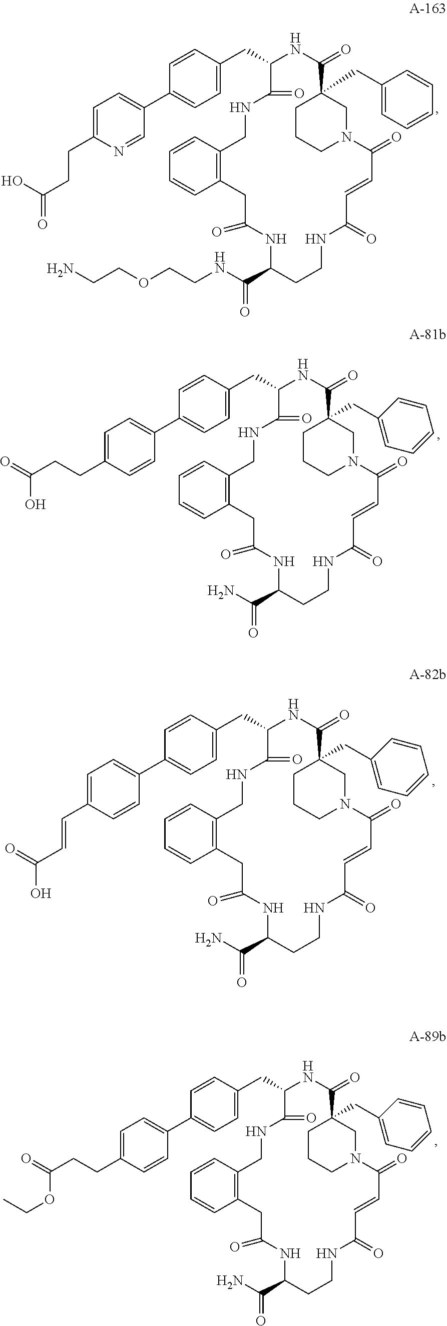

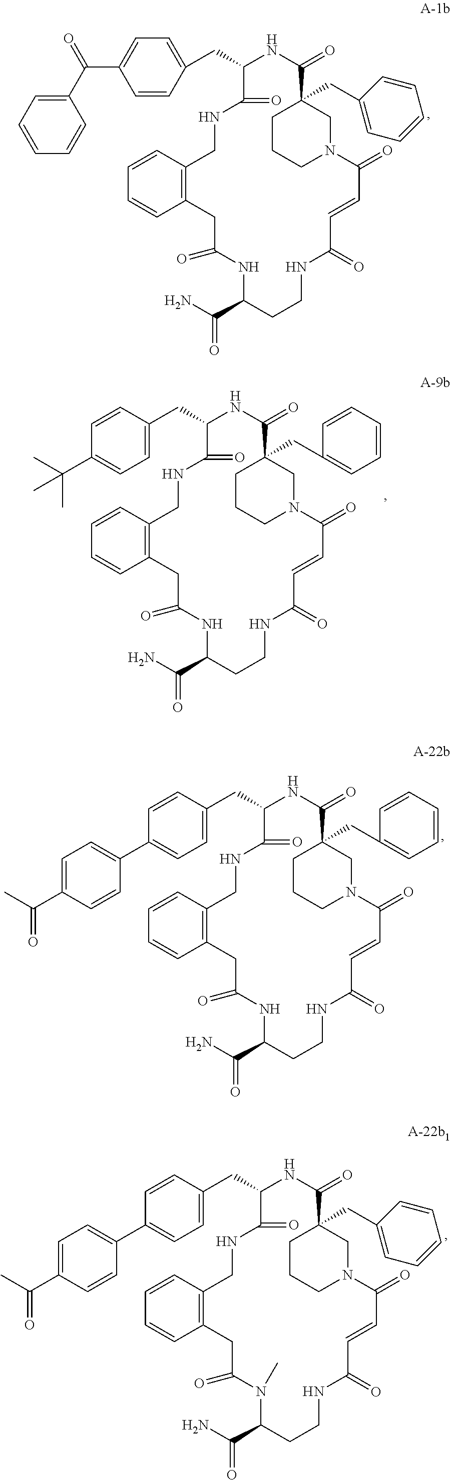 [SMILES: NC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1 ; CC(C)(C)c1ccc(C[C@@H]2NC(=O)[C@]3(Cc4ccccc4)CCCN(C3)C(=O)/C=C/C(=O)NCC[C@@H](C(N)=O)NC(=O)Cc3ccccc3CNC2=O)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(N)=O)N(C)C(=O)Cc4ccccc4CNC3=O)cc2)cc1]

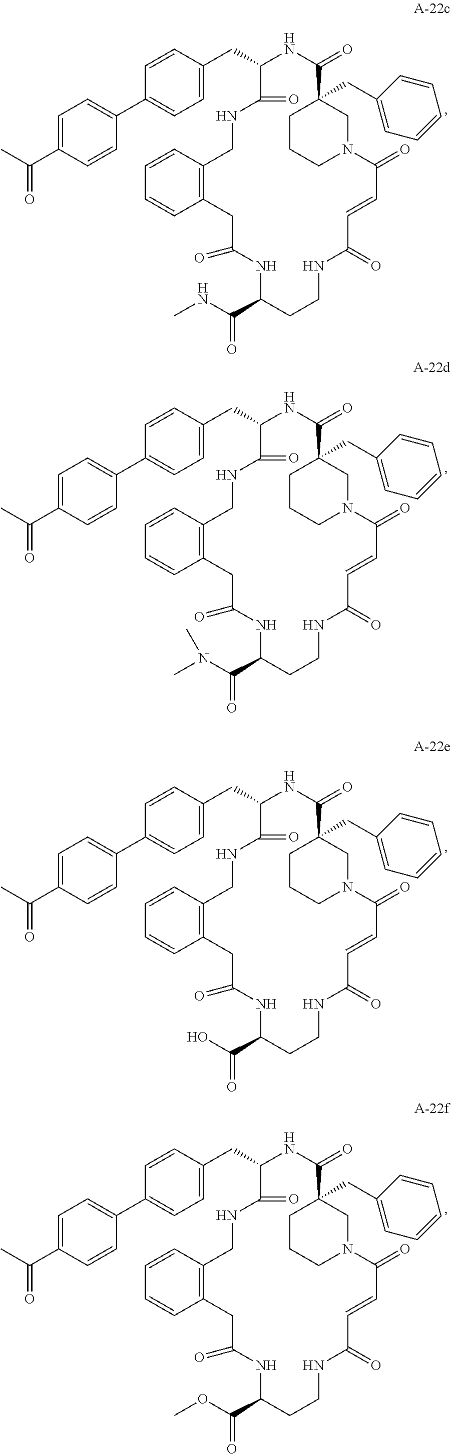 [SMILES: CNC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)N(C)C)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)O)NC(=O)Cc4ccccc4CNC3=O)cc2)cc1 ; COC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1]

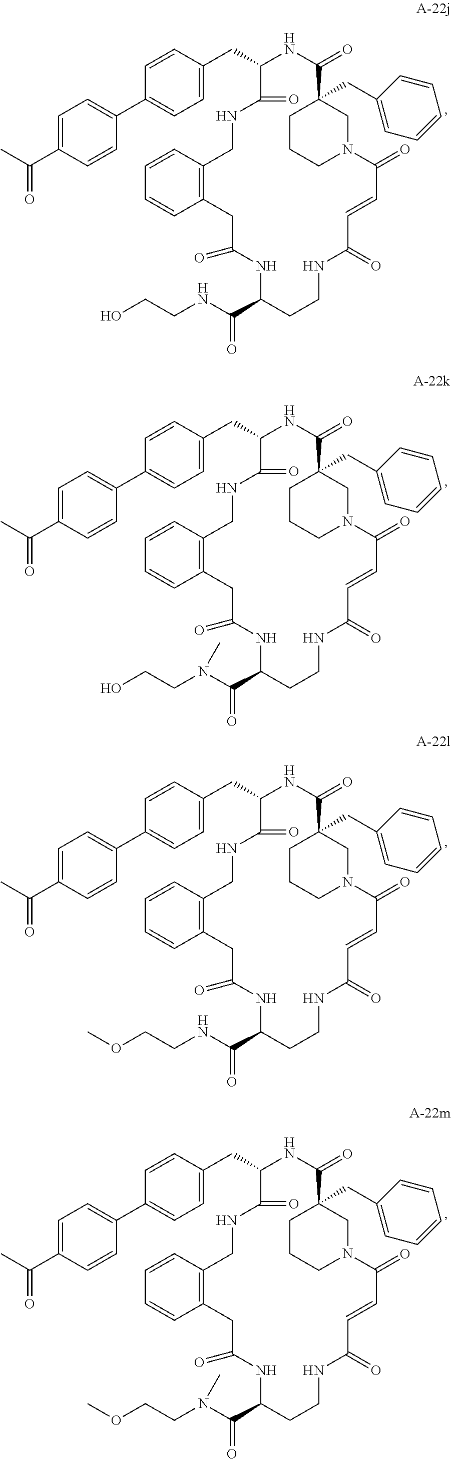

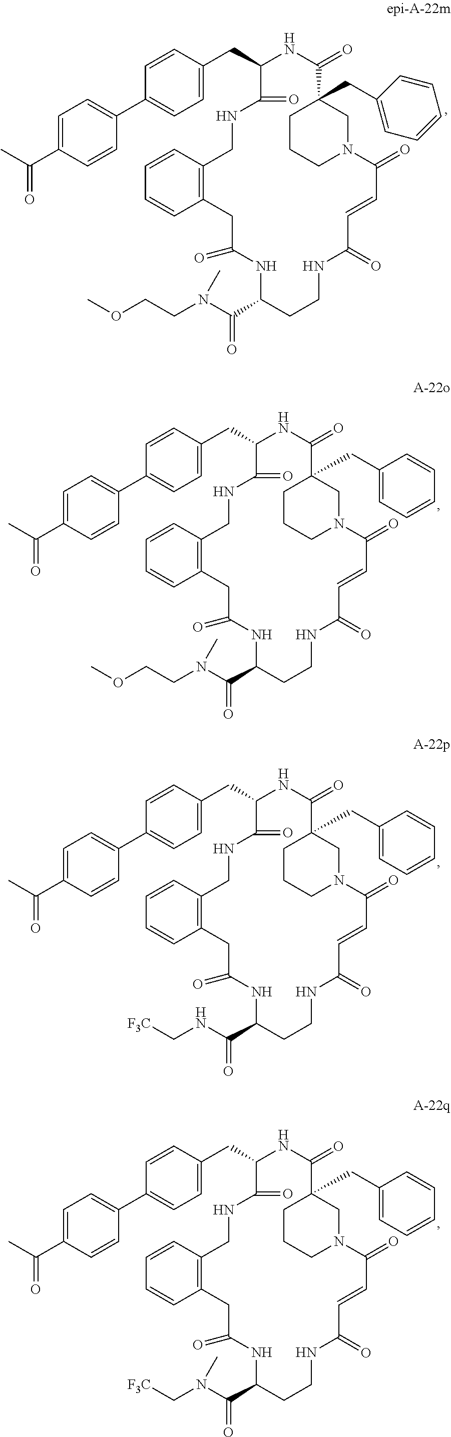

or a pharmaceutically acceptable salt, solvate, hydrate, stereoisomer, polymorph, tautomer, isotopically enriched form, or prodrug thereof.

In certain embodiments, the compound of Formula (I′) or (I) is a compound provided in any one of the Examples below. In certain embodiments, a compound described herein is a compound of Formula (I′) or (I), or a pharmaceutically acceptable salt thereof.

Certain compounds described herein bind, covalently modify, and/or inhibit a cyclophilin. In certain embodiments, the compounds described herein irreversibly inhibit a cyclophilin. In certain embodiments, the compounds described herein reversibly inhibit a cyclophilin. In certain embodiments, the cyclophilin is a cyclophilin A. In certain embodiments, the cyclophilin is cyclophilin B. In certain embodiments, the cyclophilin is cyclophilin C. In certain embodiments, the cyclophilin is cyclophilin D (CypD). In certain embodiments, the cyclophilin is cyclophilin E. In certain embodiments, the cyclophilin is cyclophilin G. In certain embodiments, the cyclophilin is cyclophilin H. In certain embodiments, the cyclophilin is cyclophilin 40. In certain embodiments, the cyclophilin is PPWD1. In certain embodiments, the cyclophilin is PPIL1. In certain embodiments, the cyclophilin is NKTR. In certain embodiments, the compounds described herein covalently bind to the cyclophilin (e.g., CypD)). In certain embodiments, the compounds described herein reversibly bind to the cyclophilin (e.g., CypB, CypC, CypD, CypE, CypG, CypH, Cyp40, PPWD1, PPIL1, NKTR). In certain embodiments, the compounds described herein non-reversibly bind to the cyclophilin (e.g., CypB, CypC, CypD, CypE, CypG, CypH, Cyp40, PPWD1, PPIL1, NKTR). In certain embodiments, the compounds described herein modulate the activity of a cyclophilin (e.g., CypB, CypC, CypD, CypE, CypG, CypH, Cyp40, PPWD1, PPIL1, NKTR). In certain embodiments, the compounds described herein inhibit the cyclophilin (e.g., CypB, CypC, CypD, CypE, CypG, CypH, Cyp40, PPWD1, PPIL1, NKTR). In certain embodiments, the compounds described herein reversibly inhibit the activity of a cyclophilin (e.g., CypB, CypC, CypD, CypE, CypG, CypH, Cyp40, PPWD1, PPIL1, NKTR).

The binding affinity of a compound described herein to a cyclophilin (e.g., CypB, CypC, CypD, CypE, CypG, CypH, Cyp40, PPWD1, PPIL1, NKTR) may be measured by the dissociation constant (K_{d}) value of an adduct of the compound and the cyclophilin (e.g., CypB, CypC, CypD, CypE, CypG, CypH, Cyp40, PPWD1, PPIL1, NKTR) using methods known in the art (e.g., isothermal titration calorimetry (ITC)). In certain embodiments, the K_{d} value of the adduct is not more than about 100 μM, not more than about 10 μM, not more than about 1 μM, not more than about 100 nM, not more than about 10 nM, or not more than about 1 nM.

In certain embodiments, the activity of a cyclophilin (e.g., CypB, CypC, CypD, CypE, CypG, CypH, Cyp40, PPWD1, PPIL1, NKTR) is inhibited by a compound described herein. The inhibition of the activity of a cyclophilin (e.g., CypB, CypC, CypD, CypE, CypG, CypH, Cyp40, PPWD1, PPIL1, NKTR) by a compound described herein may be measured by determining the half maximal inhibitory concentration (IC₅₀) of the compound when the compound, or a pharmaceutical composition thereof, is contacted with the cyclophilin (e.g., CypB, CypC, CypD, CypE, CypG, CypH, Cyp40, PPWD1, PPIL1, NKTR). The IC₅₀ values may be obtained using methods known in the art (e.g., by a competition binding assay). In certain embodiments, the IC₅₀ value of a compound described herein is not more than about 1 mM, not more than about 100 μM, not more than about 10 μM, not more than about 1 μM, not more than about 100 nM, not more than about 10 nM, or not more than about 1 nM.

The compounds described herein may selectively modulate the activity of a cyclophilin (e.g., CypB, CypC, CypD, CypE, CypG, CypH, Cyp40, PPWD1, PPIL1, NKTR). In certain embodiments, the compounds selectively increase the activity of a cyclophilin (e.g., CypB, CypC, CypD, CypE, CypG, CypH, Cyp40, PPWD1, PPIL1, NKTR). In certain embodiments, the compounds selectively inhibit the activity of a cyclophilin (e.g., CypB, CypC, CypD, CypE, CypG, CypH, Cyp40, PPWD1, PPIL1, NKTR) over other cyclophilins. In certain embodiments, the compounds inhibit the activity of two or more cyclophilins (e.g., CypB, CypC, CypD, CypE, CypG, CypH, Cyp40, PPWD1, PPIL1, NKTR) to the same extent.

The selectivity of a compound described herein in inhibiting the activity of a first cyclophilin (e.g., CypD) over a second cyclophilin may be measured by the quotient of the IC₅₀ value of the compound in inhibiting the activity of the second cyclophilin over the IC₅₀ value of the compound in inhibiting the activity of the first cyclophilin. The selectivity of a compound described herein in modulating the activity of a first cyclophilin over a second cyclophilin may also be measured by the quotient of the K_{d} value of an adduct of the compound and the second cyclophilin over the K_{d} value of an adduct of the compound and the first cyclophilin (e.g., CypD). In certain embodiments, the selectivity is at least about 1-fold, at least about 3-fold, at least about 10-fold, at least about 30-fold, at least about 100-fold, at least about 300-fold, at least about 1,000-fold, at least about 3,000-fold, at least about 10,000-fold, at least about 30,000-fold, or at least about 100,000-fold. In certain embodiments, the selectivity is at least about 1 at least 2-fold, 5-fold, 10-fold, or more. In certain embodiments, the compounds of Formula (I′) or (I) are selective for cyclophilin D compared to other cyclophilins (e.g., at least 2-fold, 5-fold, 10-fold, or more selective for cyclophilin D). In certain embodiments, the compounds of Formula (I′) or (I) are selective for cyclophilin D compared to cyclophilin E (e.g., at least 2-fold, 5-fold, 10-fold, or more selective for cyclophilin D). In certain embodiments, the compounds of Formula (I′) or (I) are selective for cyclophilin D compared to cyclophilin B (e.g., at least 2-fold, 5-fold, 10-fold, or more selective for cyclophilin D). In certain embodiments, the compounds of Formula (I′) or (I) are selective for cyclophilin D compared to cyclophilins B and/or E (e.g., at least 2-fold, 5-fold, 10-fold, or more selective for cyclophilin D). In certain embodiments, selectivity for inhibiting a first cyclophilin over other cyclophilins is measured by in vitro inhibition (IC₅₀) assays using a chymotrypsin coupled PPIase assay with Suc-AAPF-AMC as the peptide substrate was used, whereby isomerization of a peptide substrate Suc-AAPF-AMC from the cis to trans conformation allowed for proteolysis via excess a-chymotrypsin, releasing the C-terminal coumarin fluorophoreas, as disclosed in the Examples (e.g., Examples 1-3). In certain embodiments, selectivity for inhibiting a first cyclophilin over other cyclophilins is measured by Surface Plasmon Resonance (SPR) assays as disclosed in the Examples.

It is expected that the compounds described herein may be useful in treating and/or preventing diseases associated with aberrant activity (e.g., increased activity, undesired activity, abnormal activity) of a cyclophilin (e.g., CypB, CypC, CypD, CypE, CypG, CypH, Cyp40, PPWD1, PPIL1, NKTR). It is known in the art that cyclophilins are implicated in a wide range of diseases and conditions, such as neurological (e.g., neurodegenerative) diseases, metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), and conditions associated with regulation of the mitochondrial permeability transition pore (mPTP), autophagy, aging; and oxidative stress. Therefore, the compounds described herein are expected to be useful in treating and/or preventing diseases (e.g., neurological (e.g., neurodegenerative) diseases, metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), and conditions associated with regulation of the mitochondrial permeability transition pore (mPTP), autophagy, aging; and oxidative stress).

### Pharmaceutical Compositions, Kits, and Administration

The present disclosure also provides pharmaceutical compositions comprising a compound described herein and optionally a pharmaceutically acceptable excipient. In certain embodiments, a compound described herein is a compound of Formula (I′) or (I), or a pharmaceutically acceptable salt thereof, and a pharmaceutically acceptable excipient. In certain embodiments, a compound described herein in a pharmaceutical composition for treating the diseases and/or conditions described herein is a compound of Formula (I′) or (I), a compound of Table 1, or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for inhibiting the aberrant activity of a cyclophilin (e.g., CypB, CypC, CypD, CypE, CypG, CypH, Cyp40, PPWD1, PPIL1, NKTR). In certain embodiments, a therapeutically effective amount is an amount effective for treating a disease (e.g., neurological (e.g., neurodegenerative) disease, metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), and conditions associated with regulation of the mitochondrial permeability transition pore (mPTP), autophagy, aging; and oxidative stress; and other diseases associated with cyclophilins (e.g., CypD)). In certain embodiments, a therapeutically effective amount is an amount effective for inhibiting the aberrant activity of a cyclophilin (e.g., CypB, CypC, CypD, CypE, CypG, CypH, Cyp40, PPWD1, PPIL1, NKTR) and treating a disease (e.g., a disease associated with aberrant activity of a cyclophilin (e.g., CypB, CypC, CypD, CypE, CypG, CypH, Cyp40, PPWD1, PPIL1, NKTR) cyclophilin (e.g., CypB, CypC, CypD, CypE, CypG, CypH, Cyp40, PPWD1, PPIL1, NKTR). In certain embodiments, a therapeutically effective amount is an amount effective for inducing apoptosis of a cell (e.g., cell in vivo or in vitro). In certain embodiments, a prophylactically effective amount is an amount effective for inhibiting the aberrant activity of a cyclophilin (e.g., CypB, CypC, CypD, CypE, CypG, CypH, Cyp40, PPWD1, PPIL1, NKTR). In certain embodiments, a prophylactically effective amount is an amount effective for preventing or keeping a subject in need thereof in remission of a disease (e.g., a disease associated with aberrant activity of a cyclophilin (e.g., neurological (e.g., neurodegenerative) disease, metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), and conditions associated with regulation of the mitochondrial permeability transition pore (mPTP), autophagy, aging; and oxidative stress; and other diseases associated with cyclophilins (e.g., CypD)). In certain embodiments, a prophylactically effective amount is an amount effective for inhibiting the aberrant activity of a cyclophilin, and preventing or keeping a subject in need thereof in remission of a disease (e.g., a disease associated with aberrant activity of a cyclophilin (e.g., neurological (e.g., neurodegenerative) disease, metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), and conditions associated with regulation of the mitochondrial permeability transition pore (mPTP), autophagy, aging; and oxidative stress; and other diseases associated with cyclophilins (e.g., CypD)). In certain embodiments, a compound of Table 1 is used for treating diseases and/or conditions disclosed herein, provided that the compound is not used for treating cardiovascular disease, a metabolic disorder (e.g., obesity, diabetes), or a disease associated with insulin-degrading enzyme (IDE).

In certain embodiments, the effective amount is an amount effective for inhibiting the activity of a cyclophilin (e.g., CypB, CypC, CypD, CypE, CypG, CypH, Cyp40, PPWD1, PPIL1, NKTR) by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a cyclophilin (e.g., CypB, CypC, CypD, CypE, CypG, CypH, Cyp40, PPWD1, PPIL1, NKTR) by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or composition described herein is in vitro. In certain embodiments, the cell being contacted with a compound or composition described herein is in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms foral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms foral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity of a cyclophilin (e.g., CypB, CypC, CypD, CypE, CypG, CypH, Cyp40, PPWD1, PPIL1, NKTR) in a subject, biological sample, tissue, or cell), improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)). In certain embodiments, the additional pharmaceutical agent is a pharmaceutical agent useful for treating diseases associated with cyclophilins (e.g., CypD)). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is an anti-leukemia agent. In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIB O (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine I 131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPDX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC₂₂₀, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC₀₉₈₀ (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is ibrutinib. In certain embodiments, the additional pharmaceutical agent is a protein kinase inhibitor (e.g., tyrosine protein kinase inhibitor). In certain embodiments, the additional pharmaceutical agent is a binder or inhibitor of Bruton's tyrosine kinase (BTK). In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. In certain embodiments, the compounds described herein or pharmaceutical compositions can be administered in combination with a pharmaceutical agent useful for treating and/or preventing a neurological (e.g., neurodegenerative) disease (e.g., Alzheimer's disease, multiple sclerosis, Parkinson's disease, Huntington's disease). In certain embodiments, the compounds described herein or pharmaceutical compositions can be administered in combination with a pharmaceutical agent for treating and/or preventing Parkinson's disease that is levodopa, carbidopa, or a dopamine agonist. In certain embodiments, the additional pharmaceutical agent is an agent for treating Alzheimer's disease (e.g., cholinesterase inhibitors, memantine). In certain embodiments, the additional pharmaceutical agent is an agent for treating Huntington's disease (e.g., tetrabenazine). In certain embodiments, the additional pharmaceutical agent is an agent for treating amyotrophic lateral sclerosis (ALS) (e.g., glutamate blockers, edaravone). In certain embodiments, the additional pharmaceutical agent is an agent for treating multiple sclerosis (e.g., interferon beta, glatiramer acetate, CD52 antibody, sphingosine-1-phospate receptor modulators, dihydroorotate dehydrogenase (DHODH) inhibitors). In certain embodiments, the compounds described herein or pharmaceutical compositions can be administered in combination with a pharmaceutical agent useful for treating and/or preventing oxidative stress. In certain embodiments, the compounds described herein or pharmaceutical compositions can be administered in combination with a pharmaceutical agent useful for treating and/or preventing a mitochondrial disease (e.g., condition associated with modulating (e.g., regulating) the mPTP, condition related to autophagy autophagy (e.g., neurodegenerative disease, infection, cancer, aging, heart disease)). In certain embodiments, the compounds described herein or pharmaceutical compositions can be administered in combination with a pharmaceutical agent useful for treating and/or preventing a cardiovascular condition (e.g., ischemia-reperfusion injury), stroke, heart attack. In certain embodiments, the additional pharmaceutical agent is an agent for treating ischemia-reperfusion injury (e.g., blood thinners, arterial dilators).

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, metabolic disorder, autoimmune disease, or neurological disease) in a subject in need thereof. In certain embodiments, the kits are useful for preventing a disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, the kits are useful for inhibiting the activity (e.g., aberrant or unwanted activity, such as increased activity) of a cyclophilin (e.g., CypB, CypC, CypD, CypE, CypG, CypH, Cyp40, PPWD1, PPIL1, NKTR) in a subject, biological sample, tissue, or cell.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, the kits and instructions provide for preventing a disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, the kits and instructions provide for modulating (e.g., inhibiting) the activity (e.g., aberrant activity, such as increased activity) of a cyclophilin (e.g., CypB, CypC, CypD, CypE, CypG, CypH, Cyp40, PPWD1, PPIL1, NKTR) in a subject, biological sample, tissue, or cell. In certain embodiments, the kits and instructions provide for reducing oxidative stress in a subject in need thereof or in a biological sample. A kit described herein may include one or more additional pharmaceutical agents described herein as a separate composition.

### Methods of Treatment and Uses

The present disclosure provides methods of modulating (e.g., inhibiting or increasing) the activity (e.g., aberrant activity, such as increased or decreased activity) of a cyclophilin (e.g., CypB, CypC, CypD, CypE, CypG, CypH, Cyp40, PPWD1, PPIL1, NKTR). The present disclosure provides methods of modulating (e.g., inhibiting or increasing) the activity (e.g., aberrant activity, such as increased or decreased activity) of a cyclophilin (e.g., CypB, CypC, CypD, CypE, CypG, CypH, Cyp40, PPWD1, PPIL1, NKTR) in a subject, biological sample, or cell. The present disclosure provides methods of reducing oxidative stress in a subject, biological sample, or cell. The present disclosure also provides methods for the treatment of a wide range of diseases, such as diseases associated with the aberrant activity (e.g., increased activity) of a cyclophilin (e.g., CypB, CypC, CypD, CypE, CypG, CypH, Cyp40, PPWD1, PPIL1, NKTR), e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD) in a subject in need thereof. The present disclosure provides methods for the treatment and/or prevention of a neurological disease (e.g., neurodegenerative) (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD).

The present disclosure also provides a compound of Formula (I′) or (I), a compound of Table 1, or a pharmaceutically acceptable salt, co-crystal, tautomer, stereoisomer, solvate, hydrate, polymorph, isotopically enriched derivative, or prodrug, or composition thereof, for use in the treatment of a disease, such as neurological disease (e.g., neurodegenerative) (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD), in a subject in need thereof. The present disclosure also provides a compound of Formula (I′) or (I), a compound of Table 1, or a pharmaceutically acceptable salt, co-crystal, tautomer, stereoisomer, solvate, hydrate, polymorph, isotopically enriched derivative, or prodrug, or composition thereof, for use in the treatment of a disease, such as neurological disease (e.g., neurodegenerative) (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD), in a subject in need thereof. In certain embodiments, the disease and/or condition treated with a compound of Formula (I′) or (I), or a pharmaceutically acceptable salt, co-crystal, tautomer, stereoisomer, solvate, hydrate, polymorph, isotopically enriched derivative, or prodrug, or composition thereof, is not a cardiovascular disease, metabolic disorder, or a disease associated with insulin-degrading enzyme (IDE).

The present disclosure also provides uses of a compound of Formula (I′) or (I), a compound of Table 1, or a pharmaceutically acceptable salt, co-crystal, tautomer, stereoisomer, solvate, hydrate, polymorph, isotopically enriched derivative, or prodrug, or composition thereof, in the manufacture of a medicament for the treatment of a disease, such as neurological disease (e.g., neurodegenerative) (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD), in a subject in need thereof. The present disclosure also provides uses of a compound of Formula (I′) or (I), a compound of Table 1, or a pharmaceutically acceptable salt, co-crystal, tautomer, stereoisomer, solvate, hydrate, polymorph, isotopically enriched derivative, or prodrug, or composition thereof, in the manufacture of a medicament for the treatment of a disease, such as neurological disease (e.g., neurodegenerative) (e.g., Alzheimer's disease, multiple sclerosis, Parkinson's disease, Huntington's disease), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, ischemia-reperfusion injury, conditions associated with oxidative stress, mitochondrial diseases), or other diseases associated with cyclophilins (e.g., CypD), in a subject in need thereof.

In another aspect, the present disclosure provides treating a disease, such as neurological disease (e.g., neurodegenerative) (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD), the methods comprising administering to the subject an effective amount of a compound of Formula (I′) or (I), or a compound of formula:

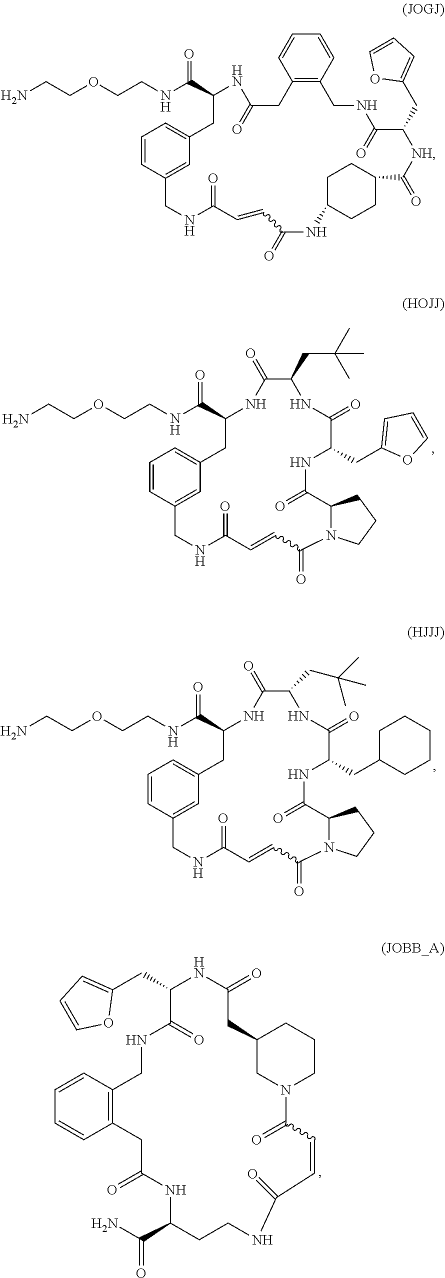 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

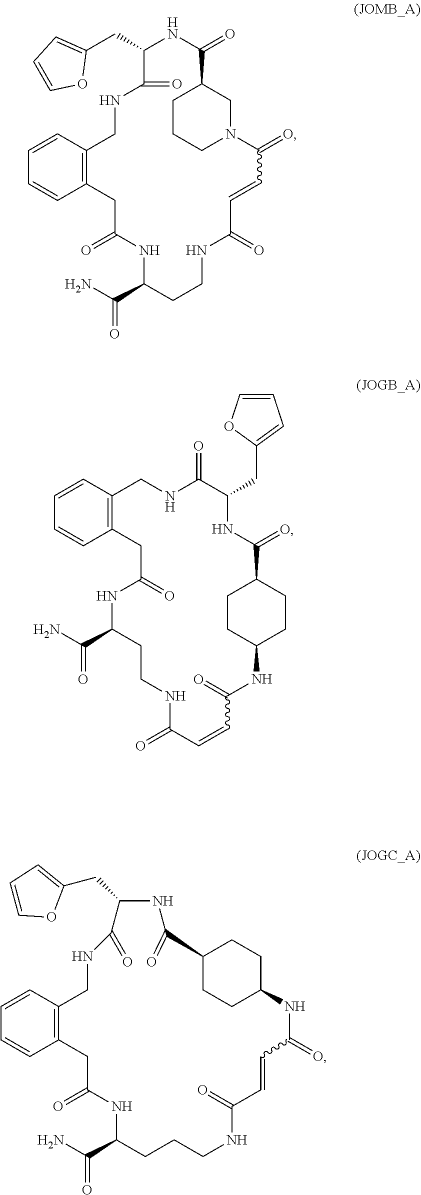 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)NCc2ccccc2CC(=O)N1]

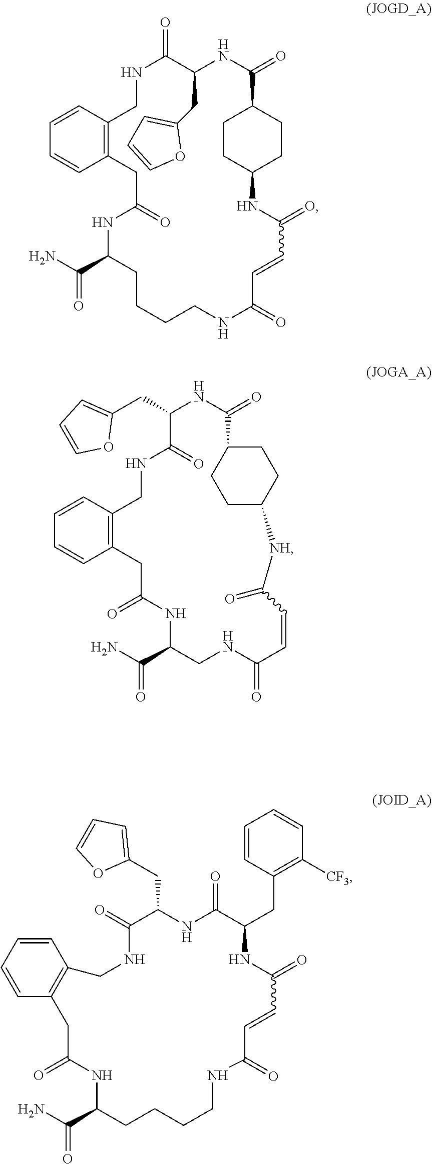 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1]

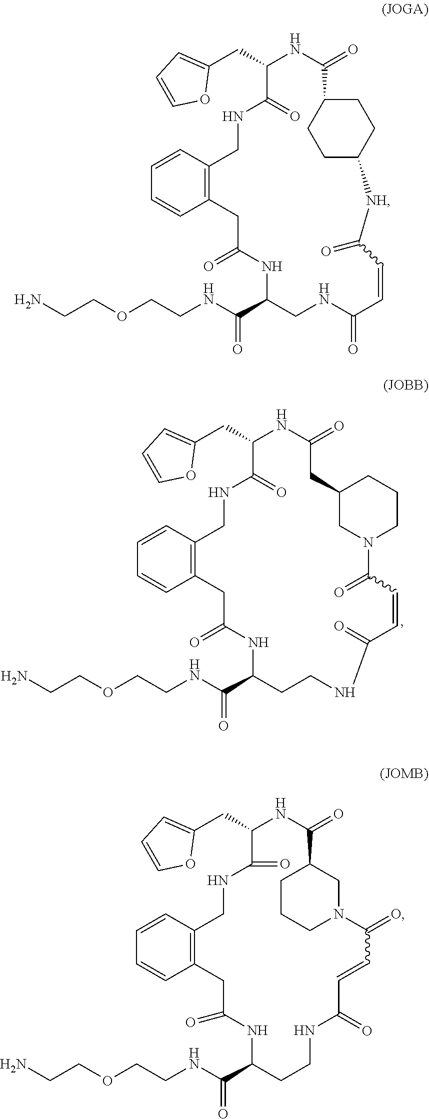 [SMILES: NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NCCOCCNC(=O)[C@@H]1CCNC(=O)C=CC(=O)N2CCC[C@H](CC(=O)N[C@@H](Cc3ccco3)C(=O)NCc3ccccc3CC(=O)N1)C2 ; NCCOCCNC(=O)[C@@H]1CCNC(=O)C=CC(=O)N2CCC[C@H](C2)C(=O)N[C@@H](Cc2ccco2)C(=O)NCc2ccccc2CC(=O)N1]

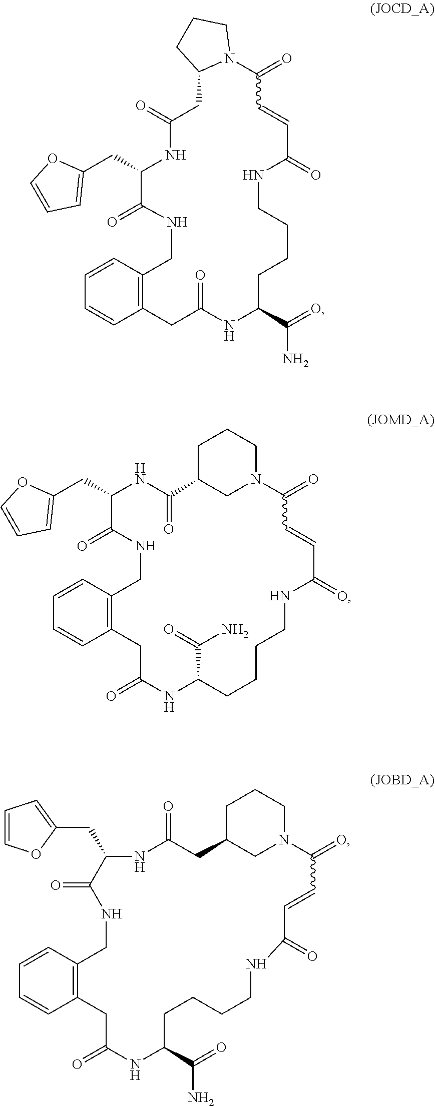 [SMILES: NC(=O)[C@@H]1CCCCNC(=O)C=CC(=O)N2CCC[C@H]2CC(=O)N[C@@H](Cc2ccco2)C(=O)NCc2ccccc2CC(=O)N1 ; NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2]

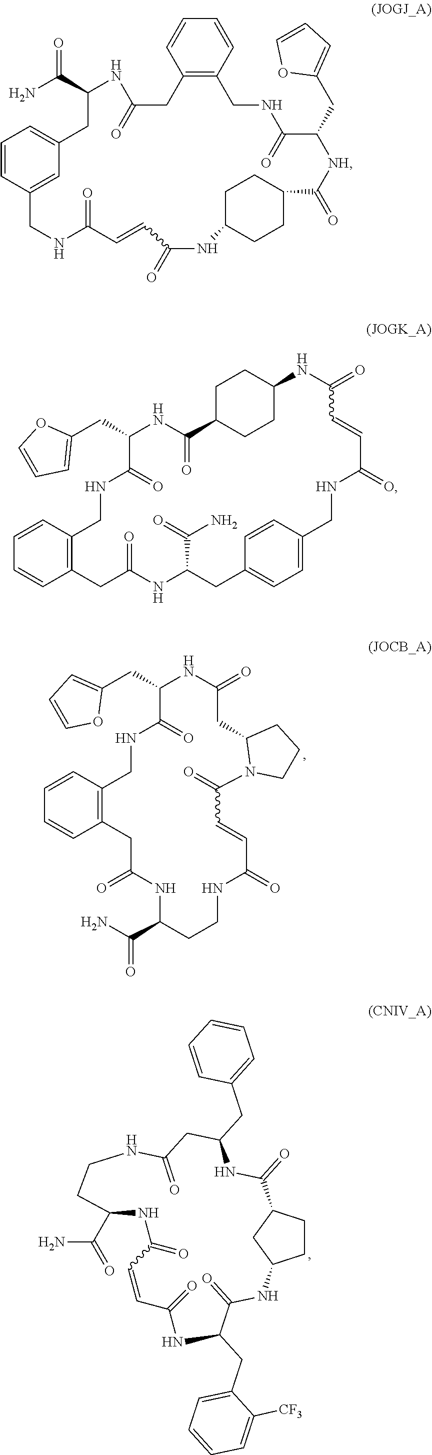 [SMILES: NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1]

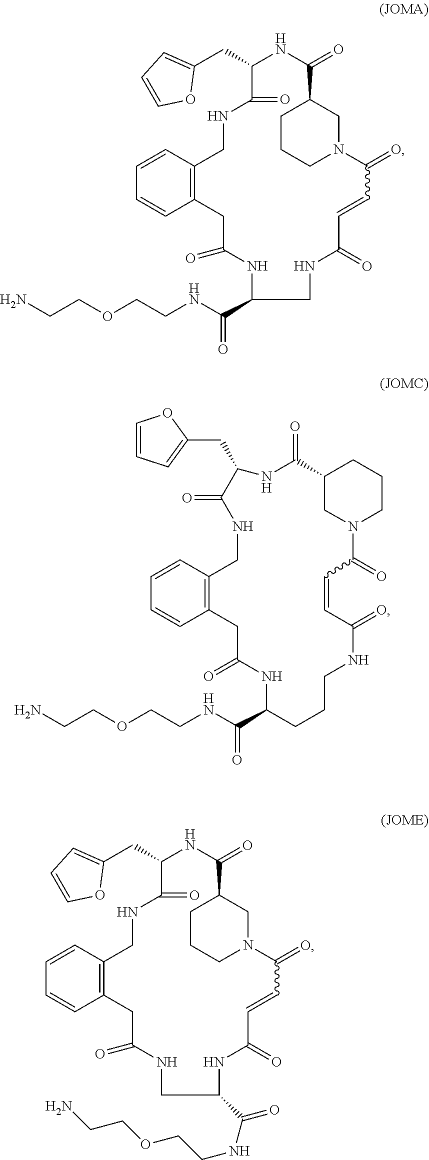 [SMILES: NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1]

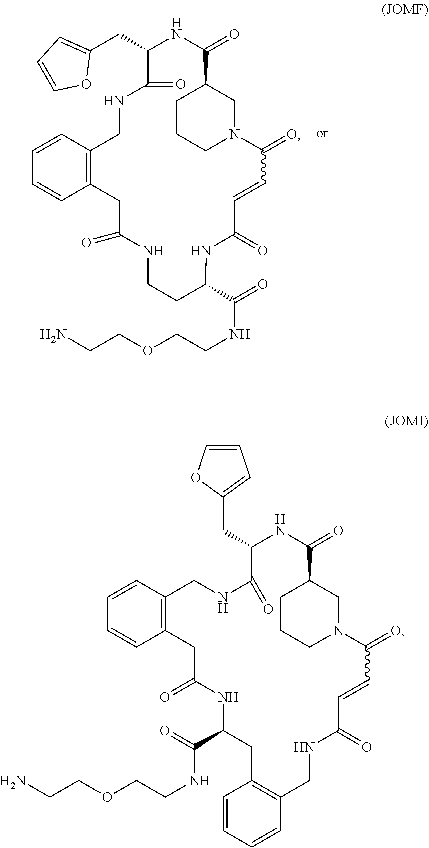 [SMILES: NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

or a pharmaceutically acceptable salt, solvate, hydrate, polymorph, co-crystal, tautomer, stereoisomer, isotopically labeled derivative, prodrug, composition, or mixture thereof, or pharmaceutical composition thereof, as described herein.

In another aspect, the present disclosure provides methods of modulating the activity of a cyclophilin (e.g., CypB, CypC, CypD, CypE, CypG, CypH, Cyp40, PPWD1, PPIL1, NKTR) in a subject, biological sample, or cell. In certain embodiments, provided are methods of inhibiting a cyclophilin in a subject. In certain embodiments, provided are methods of inhibiting a cyclophilin in a cell. In certain embodiments, provided are methods of inhibiting the activity of a cyclophilin in a subject. In certain embodiments, provided are methods of inhibiting the activity of a cyclophilin in a cell. The compounds described herein may exhibit cyclophilin inhibitory activity; the ability to inhibit a cyclophilin; the ability to inhibit CypB, without inhibiting another cyclophilin; the ability to inhibit CypC, without inhibiting another cyclophilin; the ability to inhibit CypD, without inhibiting another cyclophilin; the ability to inhibit CypE, without inhibiting another cyclophilin; the ability to inhibit CypG, without inhibiting another cyclophilin; the ability to inhibit CypH, without inhibiting another cyclophilin; the ability to inhibit Cyp40, without inhibiting another cyclophilin; the ability to inhibit PPWD1, without inhibiting another cyclophilin; the ability to inhibit PPIL1, without inhibiting another cyclophilin; the ability to inhibit NKTR, without inhibiting another cyclophilin; a therapeutic effect and/or preventative effect in the treatment of neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, the compound being administered or used inhibits a cyclophilin (e.g., CypB, CypC, CypD, CypE, CypG, CypH, Cyp40, PPWD1, PPIL1, NKTR) in a subject or cell, treats and/or prevents a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilin (e.g., CypD); and/or a therapeutic profile (e.g., optimum safety and curative effect) that is superior to existing chemotherapeutic agents, or agents for treating neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins (e.g., CypD)) in a subject in need thereof. In certain embodiments, the compound being administered or used inhibits a cyclophilin (e.g., CypB, CypC, CypD, CypE, CypG, CypH, Cyp40, PPWD1, PPIL1, NKTR) in a subject or cell, treats and/or prevents a disease (e.g., neurodegenerative disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilin (e.g., CypD).

In certain embodiments, provided are methods of decreasing the activity of a cyclophilin (e.g., CypB, CypC, CypD, CypE, CypG, CypH, Cyp40, PPWD1, PPIL1, NKTR) in a subject or biological sample (e.g., cell, tissue) by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a cyclophilin (e.g., CypB, CypC, CypD, CypE, CypG, CypH, Cyp40, PPWD1, PPIL1, NKTR) in a subject or cell is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a cyclophilin (e.g., CypB, CypC, CypD, CypE, CypG, CypH, Cyp40, PPWD1, PPIL1, NKTR) in a subject or cell is selectively inhibited by the method. In some embodiments, the activity of a cyclophilin (e.g., CypB, CypC, CypD, CypE, CypG, CypH, Cyp40, PPWD1, PPIL1, NKTR) in a subject or cell is selectively decreased by the method.

Without wishing to be bound by any particular theory, the compounds described herein are able to bind (e.g., covalently modify) the cyclophilin being inhibited. In certain embodiments, a compound described herein is able to bind (e.g., covalently modify) the cyclophilin. In certain embodiments, the compound described herein is able to covalently bind a central pocket of the cyclophilin. In certain embodiments, the compound is capable of covalently binding the S2 pocket of CyPD. In certain embodiments, the compound is capable of binding (e.g., covalently binding) the gatekeeper residues of CypD (serine and/or arginine). In certain embodiments, the compound is capable of covalently binding the gatekeeper residues of CypD (Ser81, Arg82). In certain embodiments, the compound is capable of binding (e.g., covalently binding) the gatekeeper residues of CypD (Ser81, Arg82, Serine 123, and/or Arginine 124). In certain embodiments, the compound is capable of binding (e.g., covalently binding) the gatekeeper residues of CypD (Serine 123 and/or Arginine 124). In some embodiments, the compound is capable of binding (e.g., covalently binding) the gatekeeper residues of CypD (Serine 123 and Arginine 124). In certain embodiments, the compound is capable of binding (e.g., covalently binding) the gatekeeper region of CypD (e.g., the gatekeeper residues of CypD including Serine 123 and Arginine 124). In certain embodiments, the compound is capable of binding (e.g., covalently binding) the active site, the S2 pocket, and/or the gatekeeper region of CypD (e.g., the gatekeeper residues of CypD including Serine 123 and Arginine 124). In certain embodiments, the compound is capable of binding (e.g., covalently binding) the active site, the S2 pocket, and the gatekeeper region of CypD (e.g., the gatekeeper residues of CypD including Serine 123 and Arginine 124). In certain embodiments, the compound described herein is able to selectively bind CypD over other cyclophilins. In certain embodiments, the compound described herein is able to selectively inhibit CypD over other cyclophilins. In certain embodiments, the compound is capable of covalently binding CyPD. In certain embodiments, the compound is capable of covalently modifying CypD (e.g., S2 pocket of CypD). In certain embodiments, the compound is capable of covalently modifying the S2 pocket of CyPD. In certain embodiments, the compound is capable of covalently binding CypB, In certain embodiments, the compound is capable of covalently binding CypC, In certain embodiments, the compound is capable of covalently modifying CypE. In certain embodiments, the compound is capable of covalently binding CypG. In certain embodiments, the compound is capable of covalently binding CypH. In certain embodiments, the compound is capable of covalently binding Cyp40. In certain embodiments, the compound is capable of covalently binding PPWD1. In certain embodiments, the compound is capable of covalently binding PPIL1. In certain embodiments, the compound is capable of covalently binding NKTR. In certain embodiments, the compound is capable of covalently binding CypB, CypC, CypD, CypE, CypG, CypH, Cyp40, PPWD1, PPIL1, or NKTR.

In certain embodiments, the compound is capable of covalently binding CypB. In certain embodiments, the compound is capable of covalently binding CypC, In certain embodiments, the compound is capable of covalently modifying CypE. In certain embodiments, the compound is capable of covalently modifying CypG. In certain embodiments, the compound is capable of covalently modifying CypH. In certain embodiments, the compound is capable of covalently modifying Cyp40. In certain embodiments, the compound is capable of covalently modifying PPWD1. In certain embodiments, the compound is capable of covalently modifying PPIL1. In certain embodiments, the compound is capable of covalently modifying NKTR. In certain embodiments, the compound is capable of covalently modifying CypB, CypC, CypD, CypE, CypG, CypH, Cyp40, PPWD1, PPIL1, or NKTR.

In certain embodiments, the compound is capable of non-covalently modifying CypD. In certain embodiments, the compound is capable of non-covalently inhibiting CypD. In certain embodiments, the compound is capable of non-covalently modifying CypB, CypC, CypD, CypE, CypG, CypH, Cyp40, PPWD1, PPIL1, or NKTR. In certain embodiments, the compound is capable of non-covalently inhibiting CypB, CypC, CypD, CypE, CypG, CypH, Cyp40, PPWD1, PPIL1, or NKTR.

In another aspect, the present disclosure provides methods of inhibiting the activity of a cyclophilin in a subject, the methods comprising administering to the subject an effective amount (e.g., therapeutically effective amount) of a compound, or pharmaceutical composition thereof, as described herein. In another aspect, the present disclosure provides methods of inhibiting the activity of a cyclophilin (e.g., CypB, CypC, CypD, CypE, CypG, CypH, Cyp40, PPWD1, PPIL1, NKTR) in a biological sample, the methods comprising contacting the biological sample with an effective amount of a compound, or pharmaceutical composition thereof, as described herein. In another aspect, the present disclosure provides methods of inhibiting the activity of a cyclophilin in a biological sample (e.g., tissue or cell), the methods comprising contacting the biological sample (e.g., tissue or cell) with an effective amount of a compound, or pharmaceutical composition thereof, as described herein.

In another aspect, the present disclosure provides methods of inhibiting the activity of a cyclophilin (e.g., CypB, CypC, CypD, CypE, CypG, CypH, Cyp40, PPWD1, PPIL1, NKTR) in a biological sample (e.g., tissue or cell), the methods comprising contacting the biological sample (e.g., tissue or cell) with an effective amount of a compound, or pharmaceutical composition thereof, as described herein. In another aspect, the present disclosure provides methods of inhibiting (e.g., inhibiting the activity of) a cyclophilin (e.g., CypB, CypC, CypD, CypE, CypG, CypH, Cyp40, PPWD1, PPIL1, NKTR) in a subject or biological sample, the methods comprising administering to the subject or contacting the biological sample (e.g., tissue or cell) with an effective amount of a compound of Formula (I′) or (I), or a compound of formula:

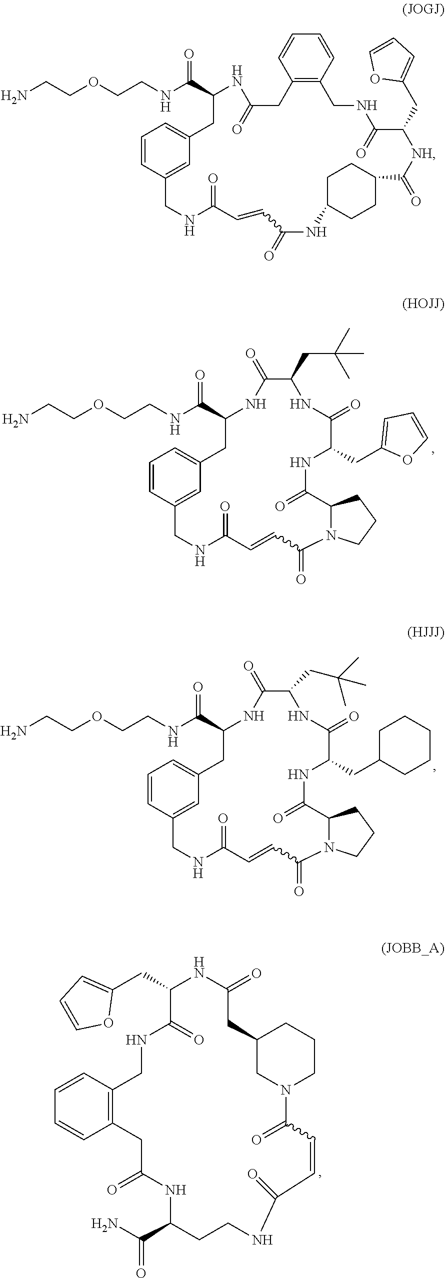 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

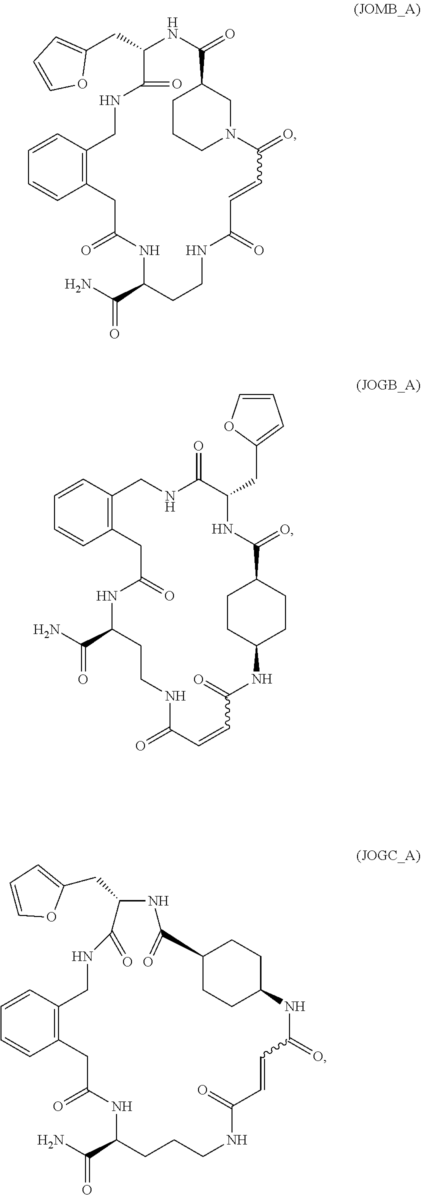 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)NCc2ccccc2CC(=O)N1]

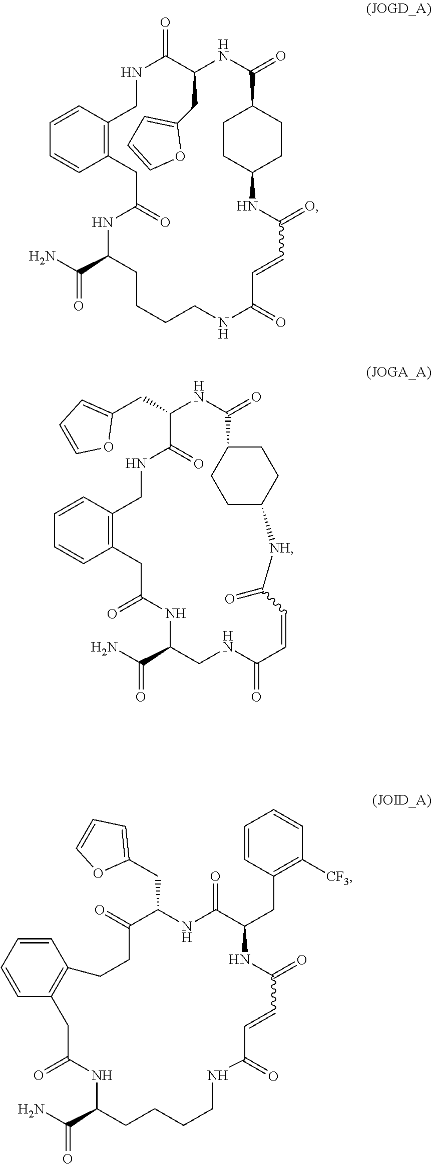 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)CCc2ccccc2CC(=O)N1]

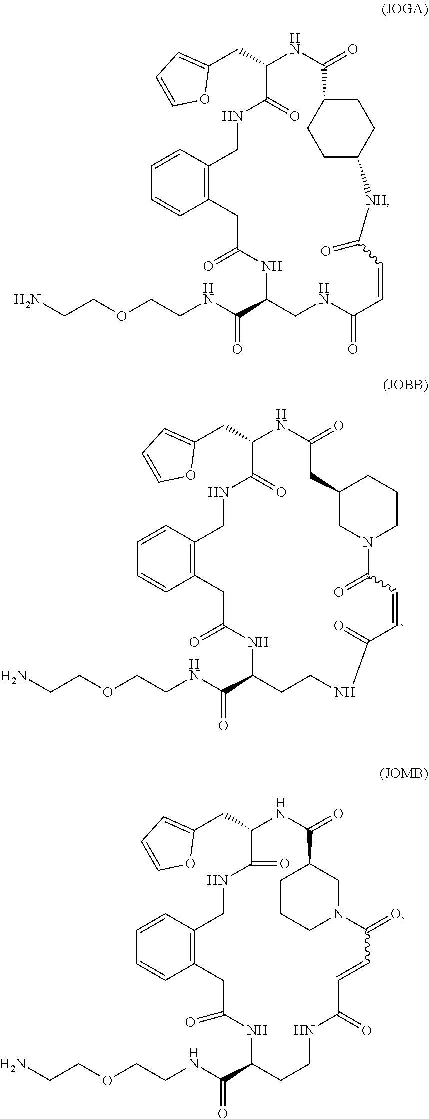 [SMILES: NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NCCOCCNC(=O)[C@@H]1CCNC(=O)C=CC(=O)N2CCC[C@H](CC(=O)N[C@@H](Cc3ccco3)C(=O)NCc3ccccc3CC(=O)N1)C2 ; NCCOCCNC(=O)[C@@H]1CCNC(=O)C=CC(=O)N2CCC[C@H](C2)C(=O)N[C@@H](Cc2ccco2)C(=O)NCc2ccccc2CC(=O)N1]

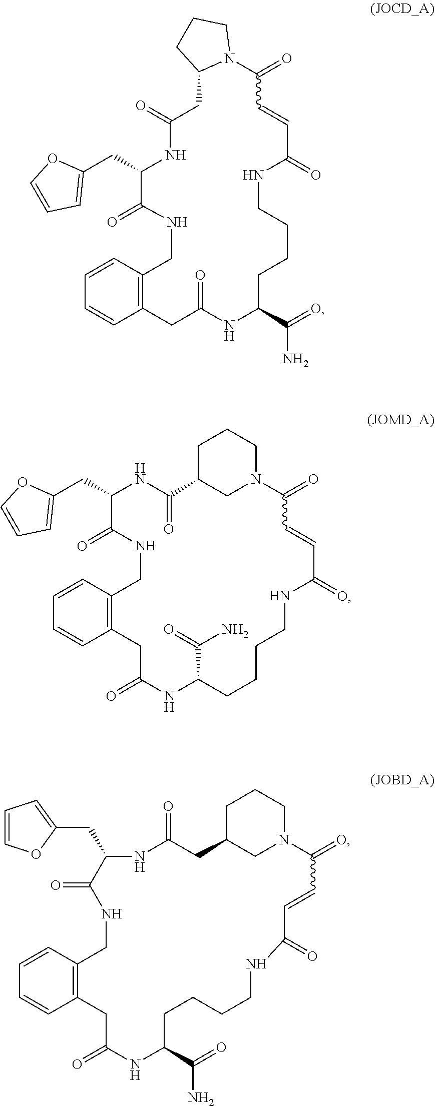 [SMILES: NC(=O)[C@@H]1CCCCNC(=O)C=CC(=O)N2CCC[C@H]2CC(=O)N[C@@H](Cc2ccco2)C(=O)NCc2ccccc2CC(=O)N1 ; NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2]

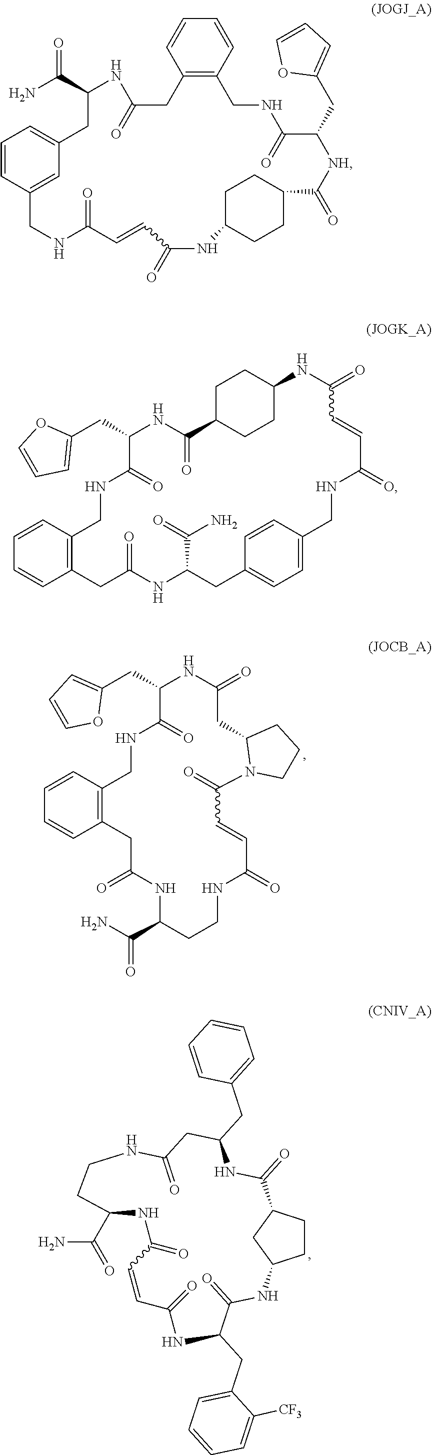 [SMILES: NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1]

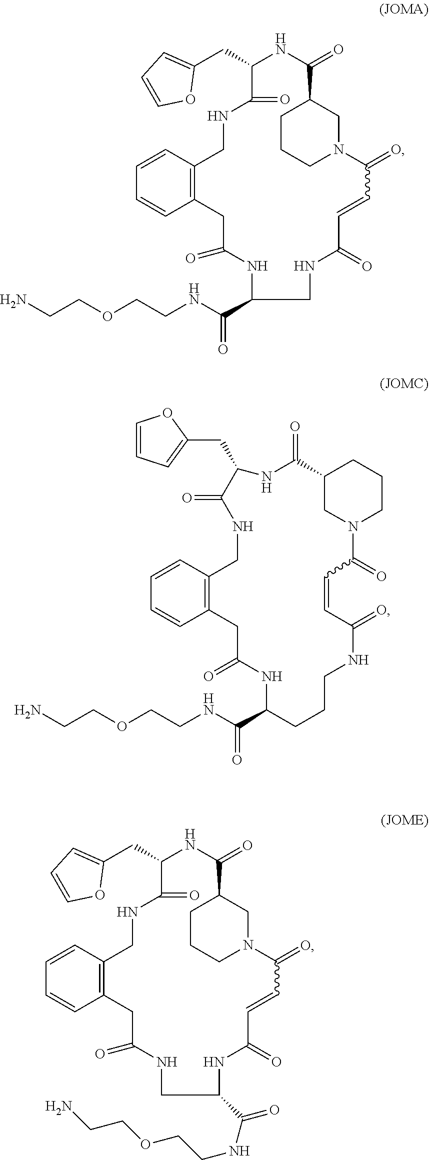 [SMILES: NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1]

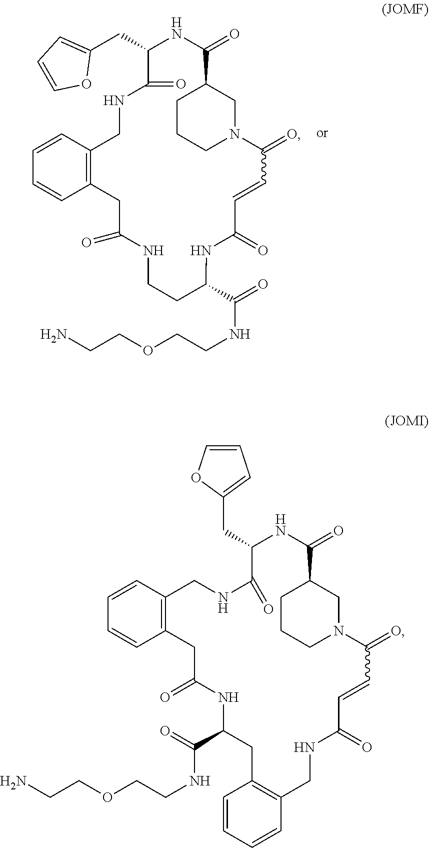 [SMILES: NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

or a pharmaceutically acceptable salt, solvate, hydrate, polymorph, co-crystal, tautomer, stereoisomer, isotopically labeled derivative, prodrug, composition, or mixture thereof, or pharmaceutical composition thereof, as described herein.

In another aspect, the present disclosure provides methods of reducing oxidative stress in a subject or biological sample, the methods comprising administering to the subject or contacting the biological sample (e.g., tissue or cell) with an effective amount of a compound, or pharmaceutical composition thereof, as described herein. In another aspect, the present disclosure provides methods of reducing oxidative stress in a subject or biological sample, the methods comprising administering to the subject or contacting the biological sample (e.g., tissue or cell) with an effective amount of a compound of Formula (I′), (I), or a compound of formula:

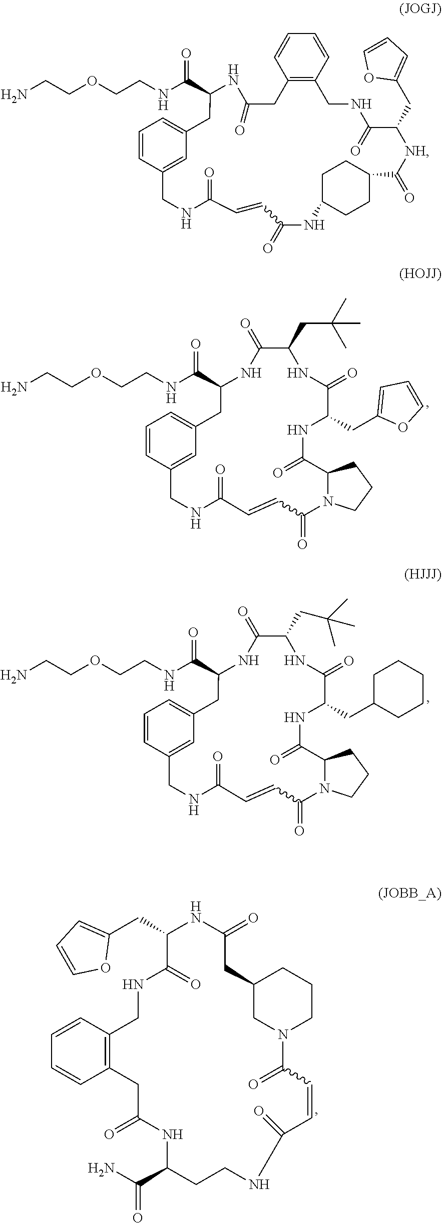 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

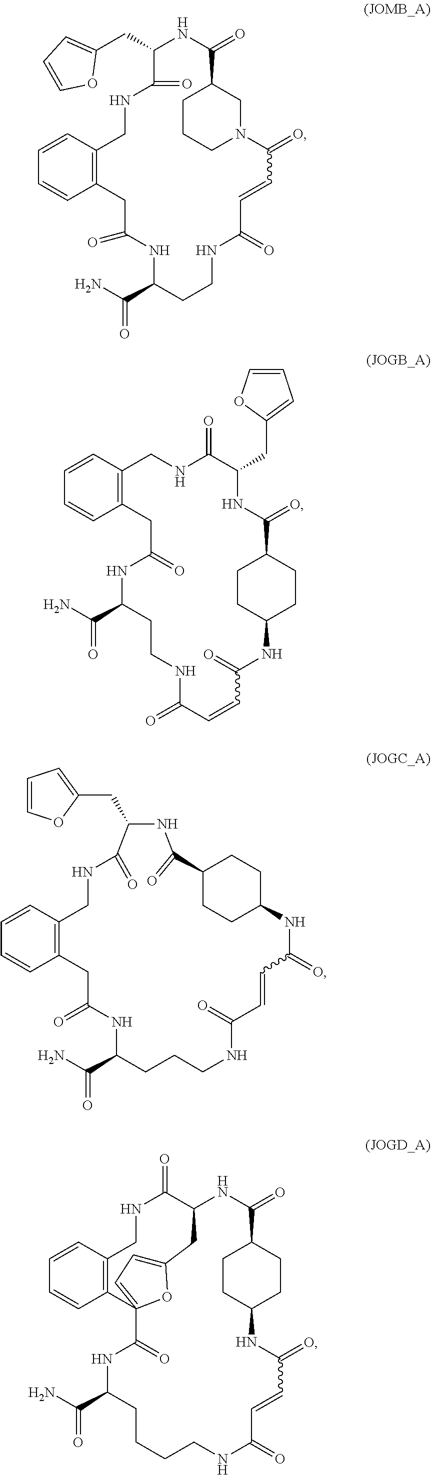

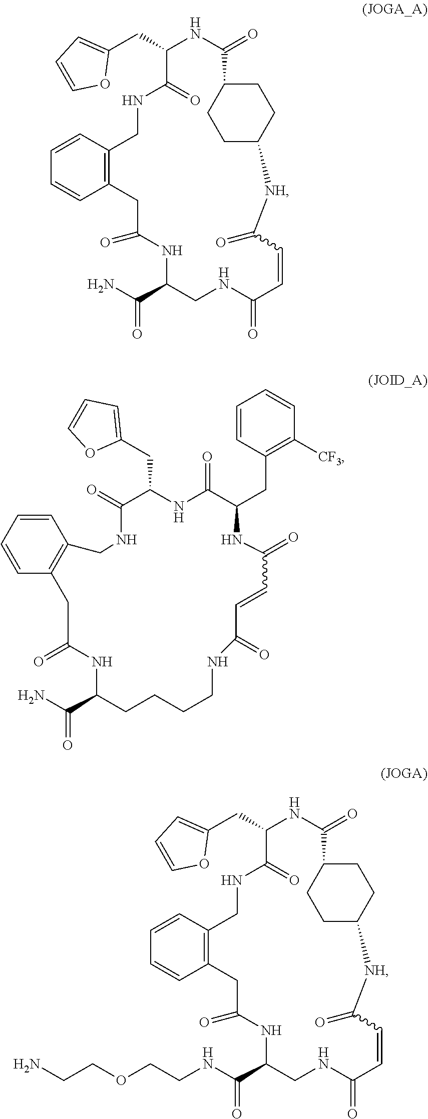 [SMILES: NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1 ; NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1]

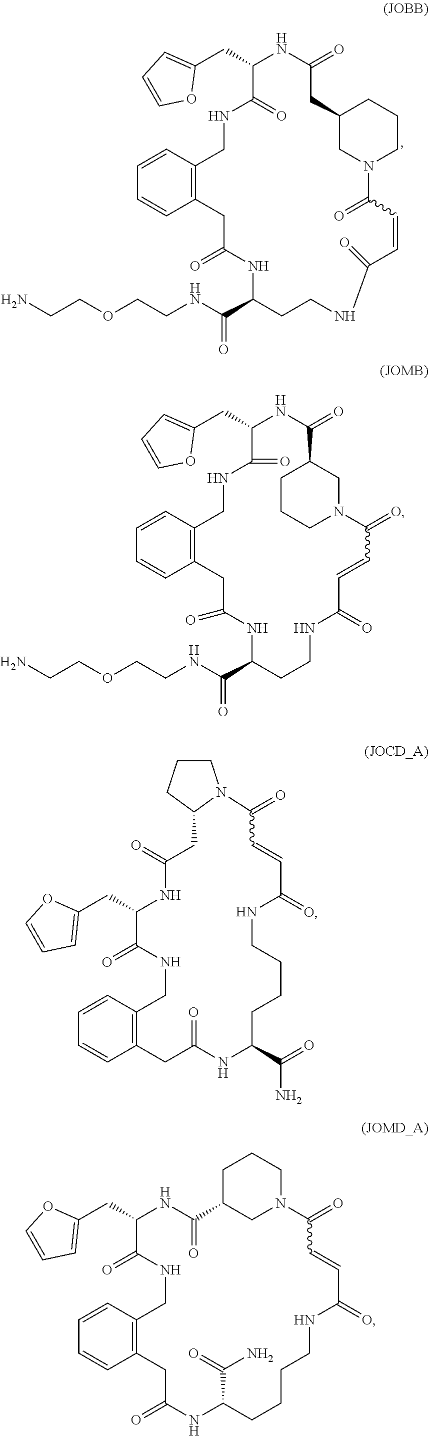

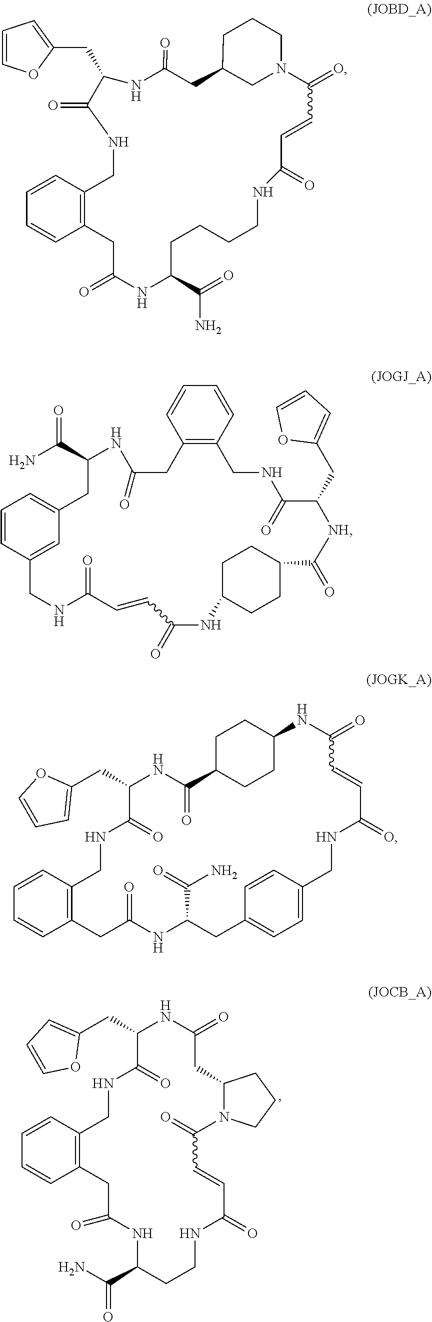 [SMILES: NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1]

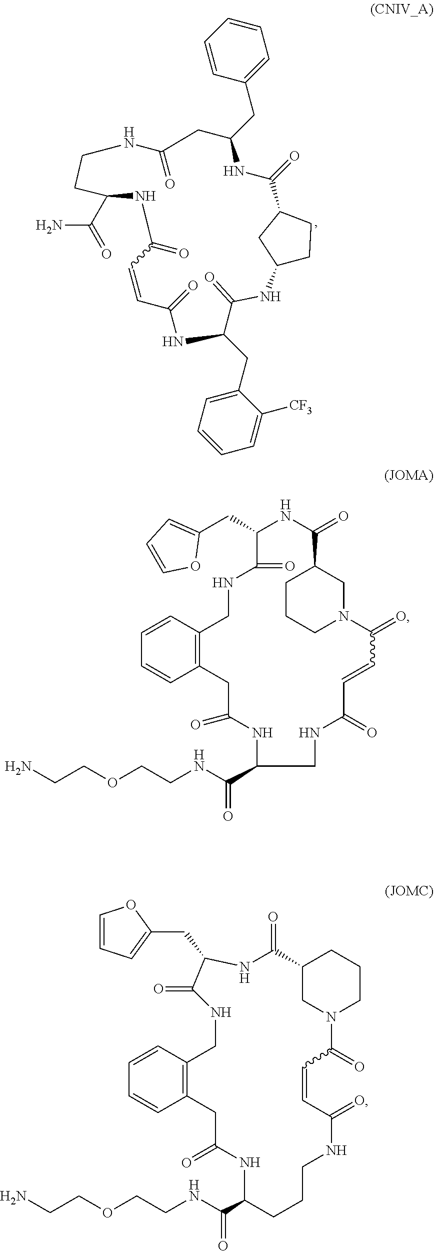 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1]

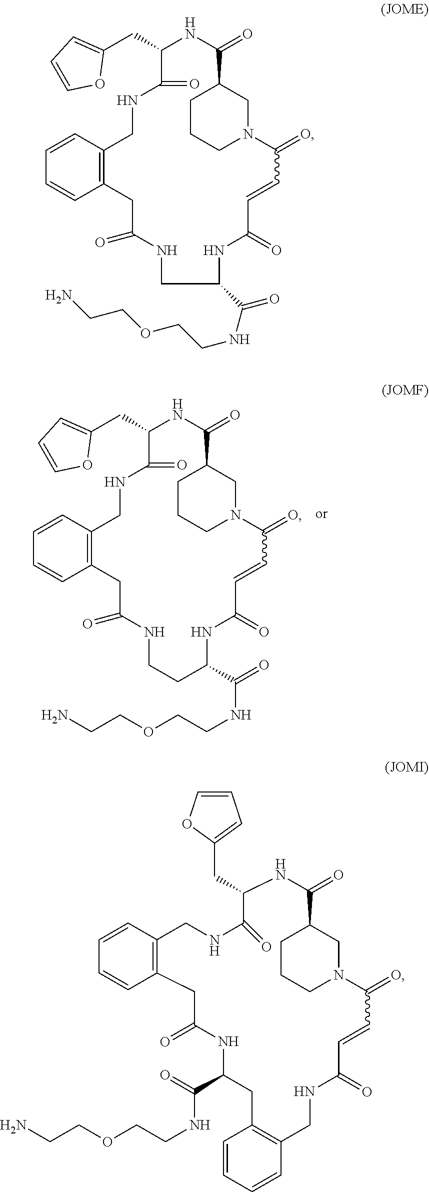 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

or a pharmaceutically acceptable salt, solvate, hydrate, polymorph, co-crystal, tautomer, stereoisomer, isotopically labeled derivative, prodrug, composition, or mixture thereof, or pharmaceutical composition thereof, as described herein. In another aspect, the present disclosure provides methods of reducing oxidative stress in a biological sample (e.g., tissue or cell), the methods comprising contacting the biological sample (e.g., tissue or cell) with an effective amount of a compound, or pharmaceutical composition thereof, as described herein.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

In certain embodiments, the biological sample being contacted with the compound or composition is breast tissue, bone marrow, lymph node, lymph tissue, spleen, or blood. In certain embodiments, the biological sample being contacted with the compound or composition is a tumor cancerous tissue. In certain embodiments, the biological sample being contacted with the compound or composition is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

In certain embodiments, the cell or tissue being contacted with the compound or composition is present in vitro. In certain embodiments, the cell or tissue being contacted with the compound or composition is present in vivo. In certain embodiments, the cell or tissue being contacted with the compound or composition is present ex vivo. In certain embodiments, the cell or tissue being contacted with the compound or composition is a malignant cell (e.g., malignant blood cell). In certain embodiments, the cell being contacted with the compound or composition is a malignant hematopoietic stem cell (e.g., malignant myeloid cell or malignant lymphoid cell). In certain embodiments, the cell being contacted with the compound or composition is a malignant lymphocyte (e.g., malignant T-cell or malignant B-cell). In certain embodiments, the cell being contacted with the compound or composition is a malignant white blood cell. In certain embodiments, the cell being contacted with the compound or composition is a malignant neutrophil, malignant macrophage, or malignant plasma cell. In certain embodiments, the cell being contacted with the compound or composition is a carcinoma cell. In certain embodiments, the cell being contacted with the compound or composition is a breast carcinoma cell. In certain embodiments, the cell being contacted with the compound or composition is a sarcoma cell. In certain embodiments, the cell being contacted with the compound or composition is a sarcoma cell from breast tissue.

The disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases) to be treated or prevented using the compounds described herein may be associated with increased activity of a cyclophilin (e.g., CypB, CypC, CypD, CypE, CypG, CypH, Cyp40, PPWD1, PPIL1, NKTR). The disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases) to be treated or prevented using the compounds described herein may be associated with the overexpression of a cyclophilin (e.g., CypB, CypC, CypD, CypE, CypG, CypH, Cyp40, PPWD1, PPIL1, NKTR).

In certain embodiments, the disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases) to be treated or prevented using the compounds described herein may be associated with the overexpression of a cyclophilin (e.g., CypB, CypC, CypD, CypE, CypG, CypH, Cyp40, PPWD1, PPIL1, NKTR). A disease (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases) may be associated with aberrant activity of a cyclophilin (e.g., CypB, CypC, CypD, CypE, CypG, CypH, Cyp40, PPWD1, PPIL1, NKTR). Aberrant activity of a cyclophilin (e.g., CypB, CypC, CypD, CypE, CypG, CypH, Cyp40, PPWD1, PPIL1, NKTR) may be elevated and/or inappropriate or undesired activity of the cyclophilin. The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a cyclophilin (e.g., CypB, CypC, CypD, CypE, CypG, CypH, Cyp40, PPWD1, PPIL1, NKTR) and be useful in treating and/or preventing diseases (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a cyclophilin and be useful in treating and/or preventing diseases (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a cyclophilin and be useful in treating and/or preventing diseases (e.g., neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases).

All types of biological samples described herein or known in the art are contemplated as being within the scope of the invention. In certain embodiments, the neurological disease to be treated or prevented using the compounds described herein is a neurodegenerative disease. In certain embodiments, the neurodegenerative disease is Alzheimer's disease. In certain embodiments, the neurodegenerative disease is multiple sclerosis. In certain embodiments, the neurological disease is Parkinson's disease. In certain embodiments, the neurological disease is Huntington's disease. In certain embodiments, the neurological disease is amyotrophic lateral sclerosis. In certain embodiments, the metabolic disorder to be treated or prevented using the compounds described herein is diabetes (e.g., Type I diabetes, Type II diabetes, gestational diabetes). In some embodiments, the metabolic disorder is hyperglycemia. In some embodiments, the metabolic disorder is hyperinsulinemia. In some embodiments, the metabolic disorder is insulin resistance. In some embodiments, the metabolic disorder is obesity.

In certain embodiments, the proliferative disease to be treated or prevented using the compounds described herein is cancer. All types of cancers disclosed herein or known in the art are contemplated as being within the scope of the invention. In certain embodiments, the proliferative disease is a hematological malignancy. In certain embodiments, the proliferative disease is a blood cancer. In certain embodiments, the proliferative disease is a hematological malignancy. In certain embodiments, the proliferative disease is leukemia. In certain embodiments, the proliferative disease is chronic lymphocytic leukemia (CLL). In certain embodiments, the proliferative disease is acute lymphoblastic leukemia (ALL). In certain embodiments, the proliferative disease is T-cell acute lymphoblastic leukemia (T-ALL). In certain embodiments, the proliferative disease is chronic myelogenous leukemia (CML). In certain embodiments, the proliferative disease is acute myeloid leukemia (AML). In certain embodiments, the proliferative disease is acute monocytic leukemia (AMoL). In certain embodiments, the proliferative disease is Waldenström's macroglobulinemia. In certain embodiments, the proliferative disease is Waldenström's macroglobulinemia associated with the MYD88 L265P somatic mutation. In certain embodiments, the proliferative disease is myelodysplastic syndrome (MDS). In certain embodiments, the proliferative disease is a carcinoma. In certain embodiments, the proliferative disease is lymphoma. In certain embodiments, the proliferative disease is T-cell lymphoma. In some embodiments, the proliferative disease is Burkitt's lymphoma. In certain embodiments, the proliferative disease is a Hodgkin's lymphoma. In certain embodiments, the proliferative disease is a non-Hodgkin's lymphoma. In certain embodiments, the proliferative disease is multiple myeloma. In certain embodiments, the proliferative disease is melanoma. In certain embodiments, the proliferative disease is colorectal cancer. In certain embodiments, the proliferative disease is colon cancer. In certain embodiments, the proliferative disease is breast cancer. In certain embodiments, the proliferative disease is recurring breast cancer. In certain embodiments, the proliferative disease is mutant breast cancer. In certain embodiments, the proliferative disease is HER2+ breast cancer. In certain embodiments, the proliferative disease is HER2− breast cancer. In certain embodiments, the proliferative disease is triple-negative breast cancer (TNBC). In certain embodiments, the proliferative disease is a bone cancer. In certain embodiments, the proliferative disease is osteosarcoma. In certain embodiments, the proliferative disease is Ewing's sarcoma. In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is non-small cell lung cancer. In some embodiments, the proliferative disease is liver cancer. In some embodiments, the proliferative disease is pancreatic cancer. In some embodiments, the proliferative disease is gastric cancer. In some embodiments, the proliferative disease is ovarian cancer. In some embodiments, the proliferative disease is ovarian cancer. In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention.

In certain embodiments, the disease and/or condition to be treated or prevented using the compounds described herein is a condition associated with the mitochondria (e.g., a mitochondrial disease). In certain embodiments, the mitochondrial disease and/or condition to be treated or prevented is associated with regulation of the mitochondrial permeability transition pore (mPTP). In certain embodiments, the mitochondrial disease and/or condition to be treated or prevented is associated with regulation of the opening and/or closing of the mPTP. In certain embodiments, the condition to be treated or prevented using the compounds described herein is a condition associated with autophagy and/or aging. In certain embodiments, the condition to be treated or prevented using the compounds described herein is a cardiovascular condition (e.g., ischemia-reperfusion injury), stroke, heart attack, conditions associated with oxidative stress, mitochondrial diseases). In certain embodiments, the cardiovascular condition is ischemia-reperfusion injury. In certain embodiments, the cardiovascular condition is stroke or heart attack.

In certain embodiments, the condition associated with autophagy to be treated or prevented using the compounds described herein is neurodegenerative disease (e.g., Alzheimer's disease, multiple sclerosis, Parkinson's disease, Huntington's disease), infection (e.g., infection by a bacteria, virus, or other microbes), cancer, condition associated with aging, or heart disease.

One aspect of the disclosure relates to methods of reducing oxidative stress subject or in a biological sample, the method comprising administering to the subject or contacting the biological sample with a therapeutically effective amount of compounds described herein.

Another aspect of the disclosure relates to methods of inhibiting the activity of a cyclophilin in a biological sample (e.g., tissue, cell), or subject. In certain embodiments, the cyclophilin is a CypB, CypC, CypD, CypE, CypG, CypH, Cyp40, PPWD1, PPIL1, NKTR. In certain embodiments, the activity of the cyclophilin is aberrant activity of the cyclophilin. In certain embodiments, the activity of the cyclophilin is increased activity of the cyclophilin. In certain embodiments, the inhibition of the activity of the cyclophilin is irreversible. In other embodiments, the inhibition of the activity of the cyclophilin is reversible. In certain embodiments, the methods of inhibiting the activity of the cyclophilin include attaching a compound described herein to the cyclophilin (e.g., CypB, CypC, CypD, CypE, CypG, CypH, Cyp40, PPWD1, PPIL1, NKTR). In certain embodiments, the methods comprise covalently inhibiting a cyclophilin In certain embodiments, the methods comprise covalently inhibiting a cyclophilin (e.g., CypD). In certain embodiments, the methods comprise reversibly inhibiting a cyclophilin (e.g., CypB, CypC, CypD, CypE, CypG, CypH, Cyp40, PPWD1, PPIL1, NKTR).

In certain embodiments, the methods described herein include administering to a subject or contacting a biological sample with an effective amount of a compound described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the methods described herein include administering to a subject or contacting a biological sample with an effective amount of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein. The additional pharmaceutical agent may be an agent for treating a neurological (e.g., neurodegenerative) disease. The additional pharmaceutical agent may be an agent for treating a metabolic disorder. The additional pharmaceutical agent may be an anti-aging agent. The additional pharmaceutical agent may be an agent for treating a cardiovascular condition (e.g., ischemia-reperfusion injury), stroke, heart attack, conditions associated with oxidative stress, mitochondrial diseases). The additional pharmaceutical agent may be an anti-proliferative agent. In certain embodiments, the additional pharmaceutical agent is an anti-cancer agent. The additional pharmaceutical agent may also be a cyclophilin inhibitor. In certain embodiments, the additional pharmaceutical agent is an inhibitor of CypD. In certain embodiments, the additional pharmaceutical agent is an inhibitor of a cyclophilin (e.g., CypB, CypC, CypD, CypE, CypG, CypH, Cyp40, PPWD1, PPIL1, NKTR). In certain embodiments, the additional pharmaceutical agent is a selective inhibitor of cyclophilin (e.g., CypB, CypC, CypD, CypE, CypG, CypH, Cyp40, PPWD1, PPIL1, NKTR). In certain embodiments, the additional pharmaceutical agent is a non-selective inhibitor of cyclophilin (e.g., CypB, CypC, CypD, CypE, CypG, CypH, Cyp40, PPWD1, PPIL1, NKTR)

In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. Exemplary chemotherapeutic agents include alkylating agents such as nitrogen mustards, ethylenimines, methylmelamines, alkyl sulfonates, nitrosuoureas, and triazenes; antimetabolites such as folic acid analogs, pyrimidine analogs, in particular fluorouracil and cytosine arabinoside, and purine analogs; natural products such as vinca alkaloids epi-podophyllotoxins, antibiotics, enzymes, and biological response modifiers; and miscellaneous products such as platinum coordination complexes, anthracenedione, substituted urea such as hydroxyurea, methyl hydrazine derivatives, and adrenocorticoid suppressant. Exemplary chemotherapeutic agents also include anthracycline antibiotics, actinomycin D, plicamycin, puromycin, gramicidin D, paclitaxel, colchicine, cytochalasin B, emetine, maytansine, amsacrine, cisplatin, carboplatin, mitomycin, altretamine, cyclophosphamide, lomustine, and carmustine. In certain embodiments, a pharmaceutical composition described herein further comprises a combination of the additional pharmaceutical agents described herein.

The inventive compounds or compositions may synergistically augment inhibition of cyclophilins induced by the additional pharmaceutical agent(s) in the biological sample or subject. Thus, the combination of the inventive compounds or compositions and the additional pharmaceutical agent(s) may be useful in treating proliferative diseases resistant to a treatment using the additional pharmaceutical agent(s) without the inventive compounds or compositions.

In some embodiments, the activity of a cyclophilin is non-selectively inhibited by the compounds or pharmaceutical compositions described herein. In some embodiments, the activity of the cyclophilin being inhibited is selectively inhibited by the compounds or pharmaceutical compositions described herein, compared to the activity of a cyclophilin (e.g., a different cyclophilin). In certain embodiments, the activity of a cyclophilin (e.g., CypB, CypC, CypD, CypE, CypG, CypH, Cyp40, PPWD1, PPIL1, NKTR) is selectively inhibited by a compound or pharmaceutical composition described herein, compared to the activity of a different protein. In certain embodiments, the activity of CypD is selectively inhibited by a compound or pharmaceutical composition described herein, compared to the activity of another cyclophilin (e.g., CypB, CypC, CypE, CypG, CypH, Cyp40, PPWD1, PPIL1, NKTR). In certain embodiments, the activity of a cyclophilin (e.g., CypB, CypC, CypD, CypE, CypG, CypH, Cyp40, PPWD1, PPIL1, NKTR) is selectively inhibited by a compound or pharmaceutical composition described herein, compared to the activity of another cyclophilin.

The selectivity of a compound or pharmaceutical composition described herein in inhibiting the activity of a cyclophilin over a different protein (e.g., a different cyclophilin) may be measured by the quotient of the IC₅₀ value of the compound or pharmaceutical composition in inhibiting the activity of the different protein over the IC₅₀ value of the compound or pharmaceutical composition in inhibiting the activity of the cyclophilin. The selectivity of a compound or pharmaceutical composition described herein for a cyclophilin over a different protein may also be measured by the quotient of the K_{d} value of an adduct of the compound or pharmaceutical composition and the different protein over the K_{d} value of an adduct of the compound or pharmaceutical composition and the cyclophilin. In certain embodiments, the selectivity is at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

In certain embodiments, a kit described herein includes a first container comprising a compound or pharmaceutical composition described herein. In certain embodiments, a kit described herein is useful in treating and/or preventing a disease, such as a neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilins, in a subject in need thereof, inhibiting the activity of a cyclophilin (e.g., CypB, CypC, CypD, CypE, CypG, CypH, Cyp40, PPWD1, PPIL1, NKTR) in a subject, biological sample, tissue, or cell, and/or reducing oxidative stress in a subject or in a biological sample.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a proliferative disease in a subject in need thereof, preventing a disease, such as a neurological (e.g., neurodegenerative) disease (e.g., Alzheimer's disease, multiple sclerosis, Parkinson's disease, Huntington's disease), metabolic disorder (e.g., obesity, diabetes), proliferative disease (e.g., cancers), condition associated with autophagy (e.g., neurodegenerative disease, infection, cancer, condition associated with aging, heart disease), condition associated with aging, condition associated with modulating (e.g., regulating) the mPTP, cardiovascular condition (e.g., ischemia-reperfusion injury), stroke, heart attack, conditions associated with oxidative stress, mitochondrial diseases), or other diseases associated with cyclophilinsin a subject in need thereof, inhibiting the activity of a cyclophilin (e.g., CypB, CypC, CypD, CypE, CypG, CypH, Cyp40, PPWD1, PPIL1, NKTR) in a subject, biological sample, tissue, or cell, and/or reducing oxidative stress in a subject or in a biological sample. A kit described herein may include one or more additional pharmaceutical agents described herein as a separate composition.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope. The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Compounds of Formula (I′) or (I) and/or Table 1 (shown above) may be prepared using synthetic schemes and procedures recognized by one of ordinary skill in the art.

### Example 1. Synthesis of Exemplary Cyclophilin Inhibitor Compounds

Compounds of Formula (I′) or (I) and/or Table 1 (shown above) may be prepared using the following synthetic schemes. Disclosed below is are general synthetic scheme for preparing macrocycles disclosed herein via solid phase peptide synthesis (Scheme 1), for preparing Fmoc-protected and Alloc-protected amino acids (Scheme 2), for synthesizing biphenyl building blocks or compound 49-derivatives (A-series compounds) (Scheme 3), and for synthesizing piperidine analogs (Scheme 4).

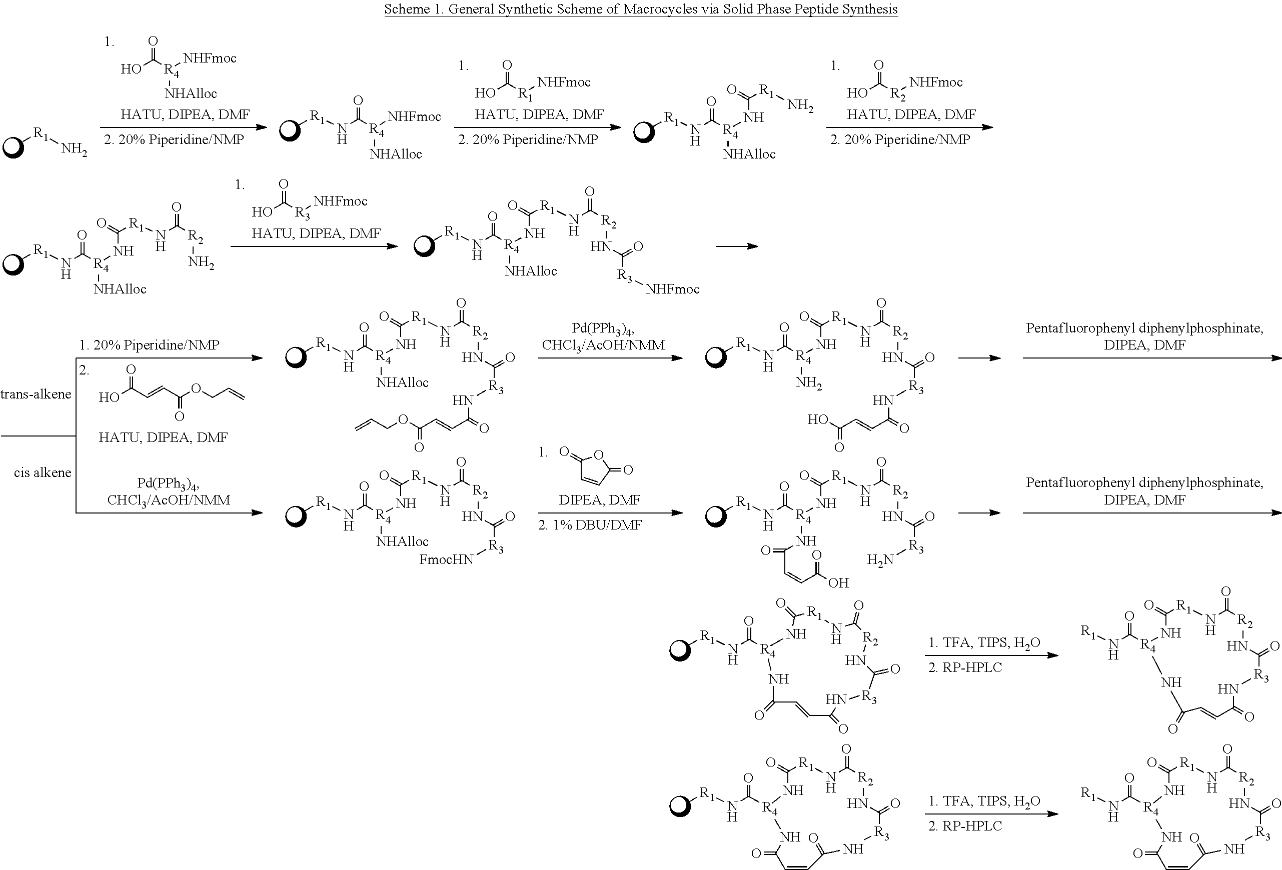

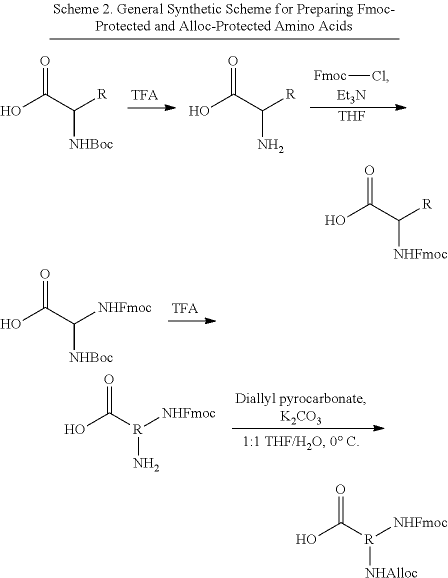

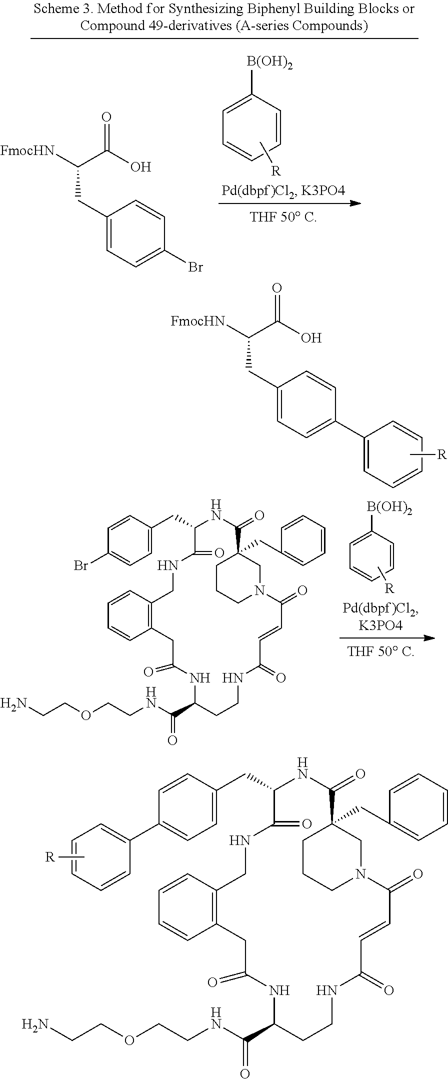

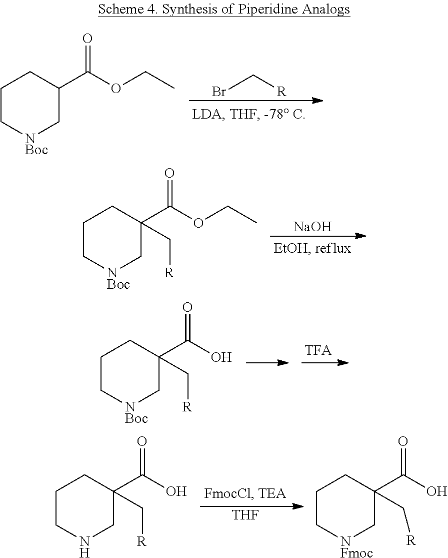

### Example 2. Biological Assays of Compounds

Novel cyclophilin inhibitors were discovered from a DNA-templated library of 256,000 macrocycles. Lead macrocycle scaffolds from the library were synthetically modified to increase both potency and specificity for CypD. The 3D structures and binding of lead hits to CypD were determined via X-ray crystallography, which were utilized to develop further improvements. Good hits from this series possessed the most specific inhibition profile for CypD over other cyclophilin family members in the reported literature while retaining very high potency.

### Results

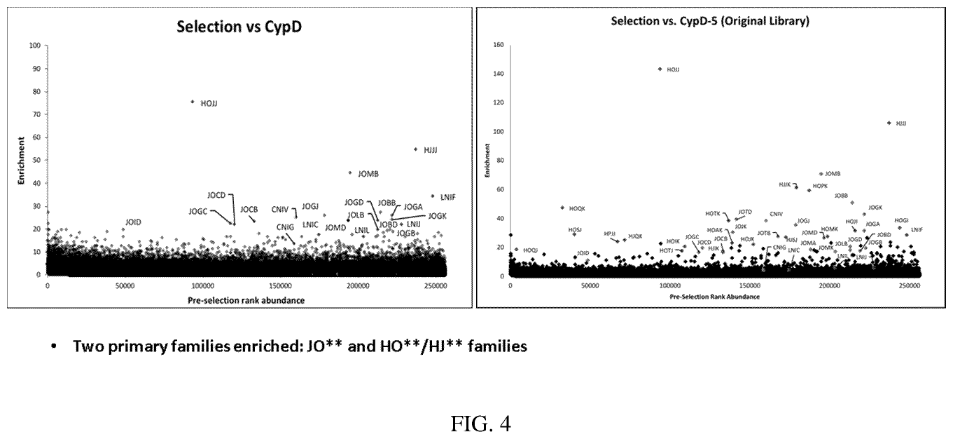

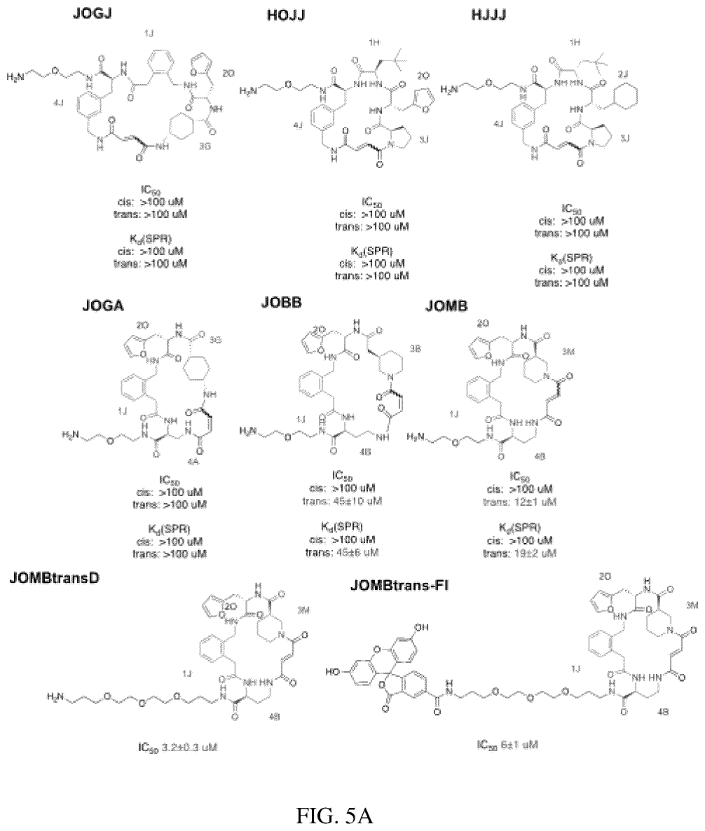

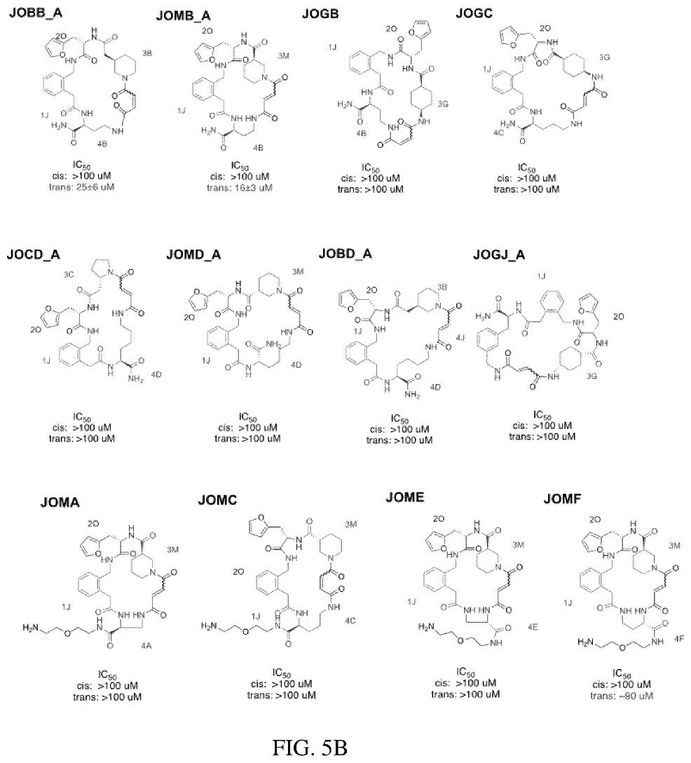

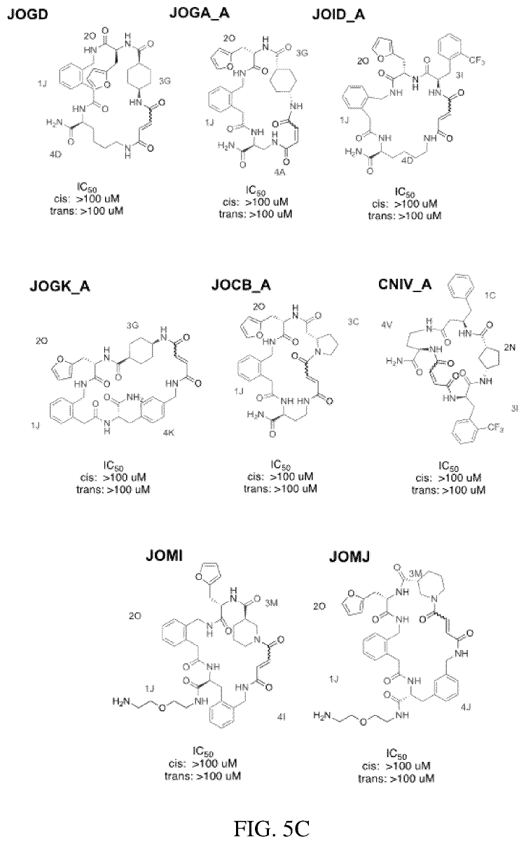

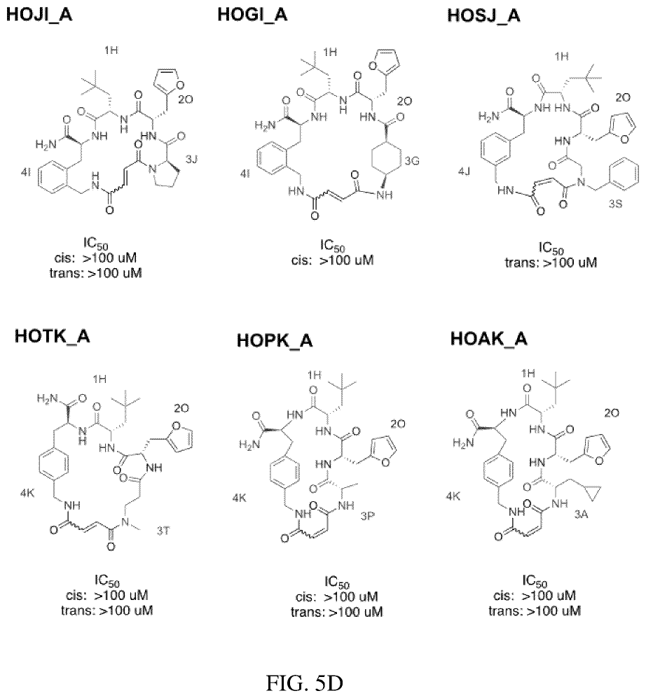

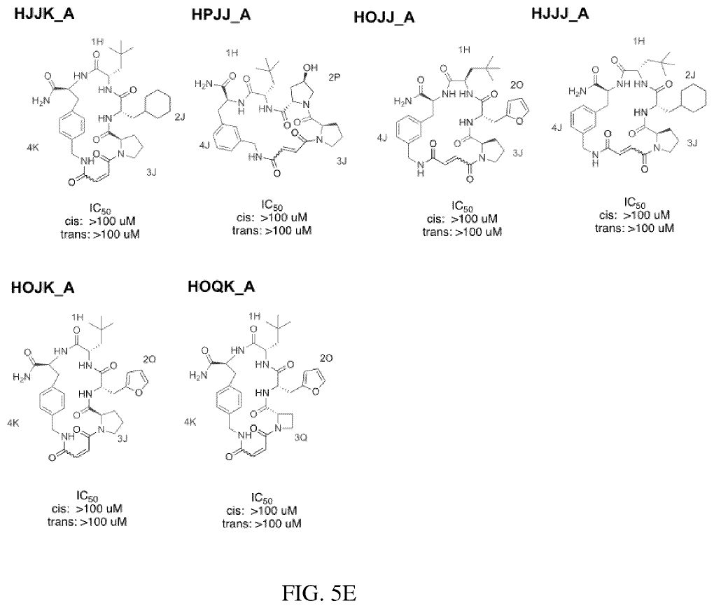

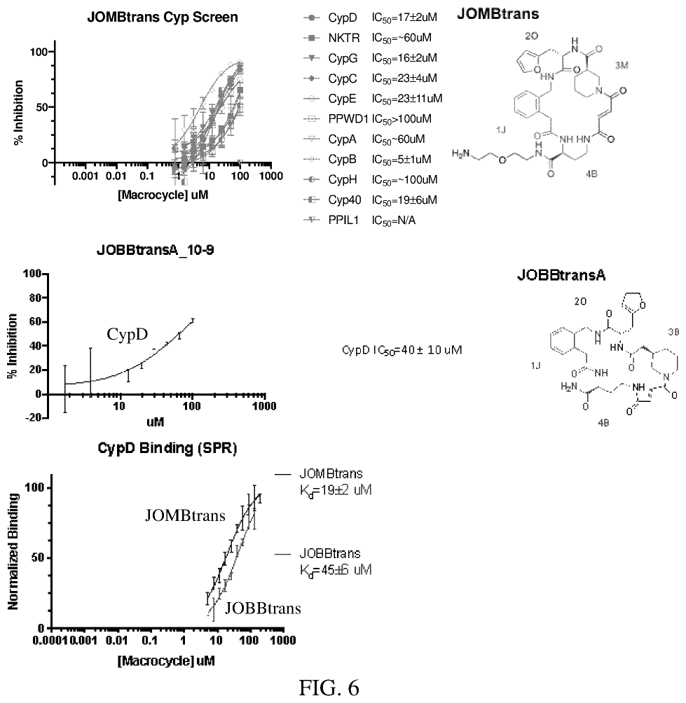

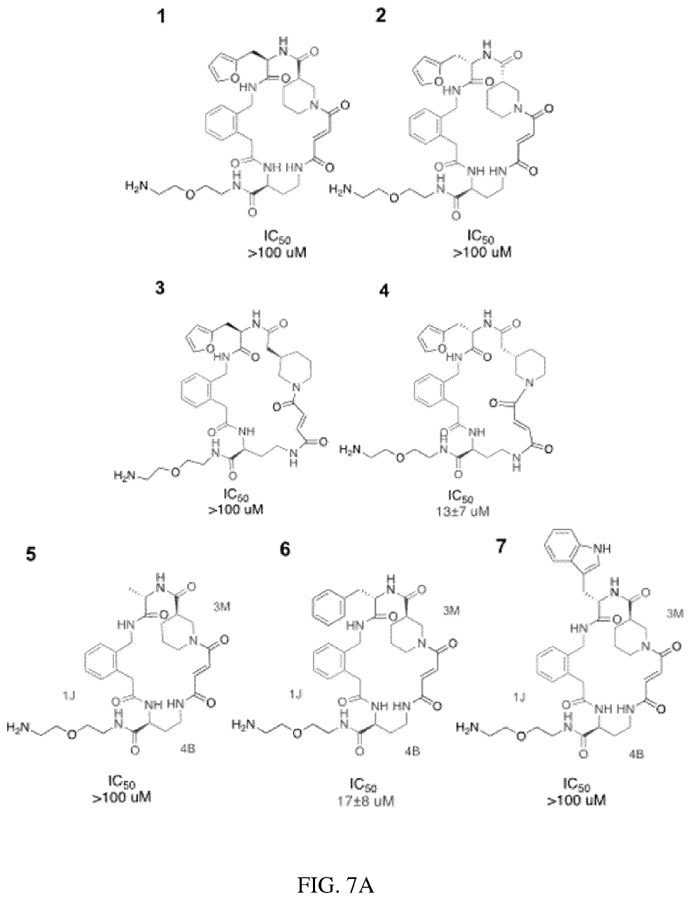

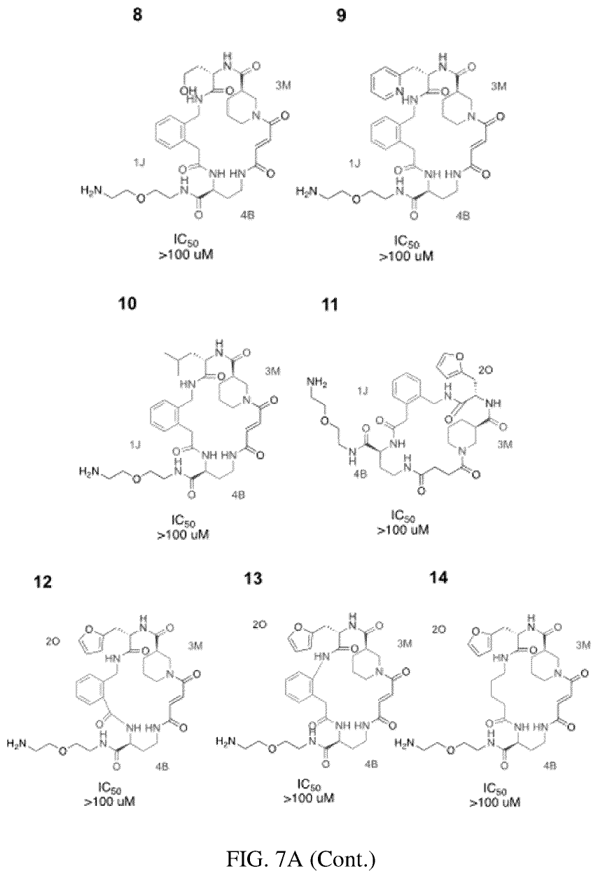

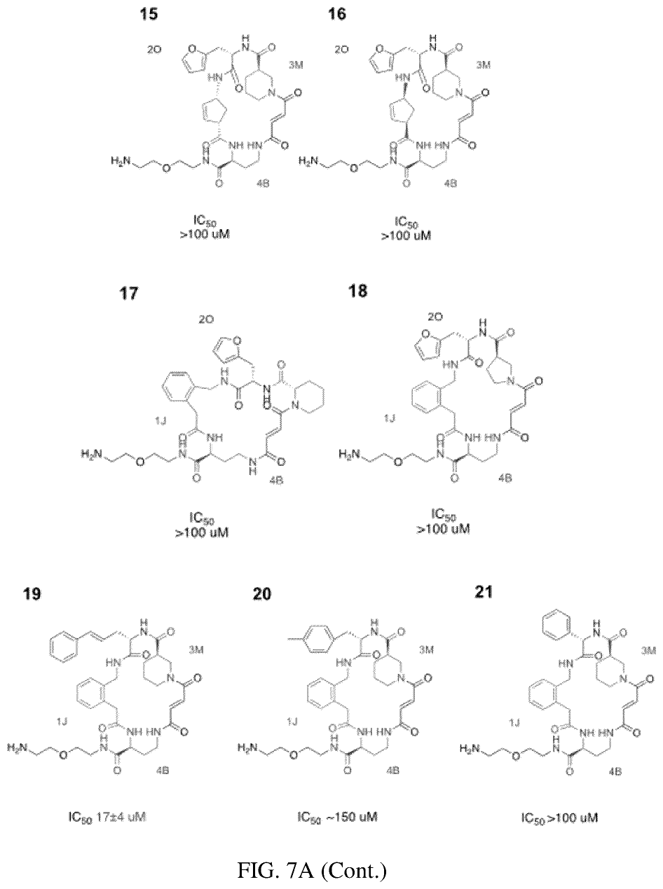

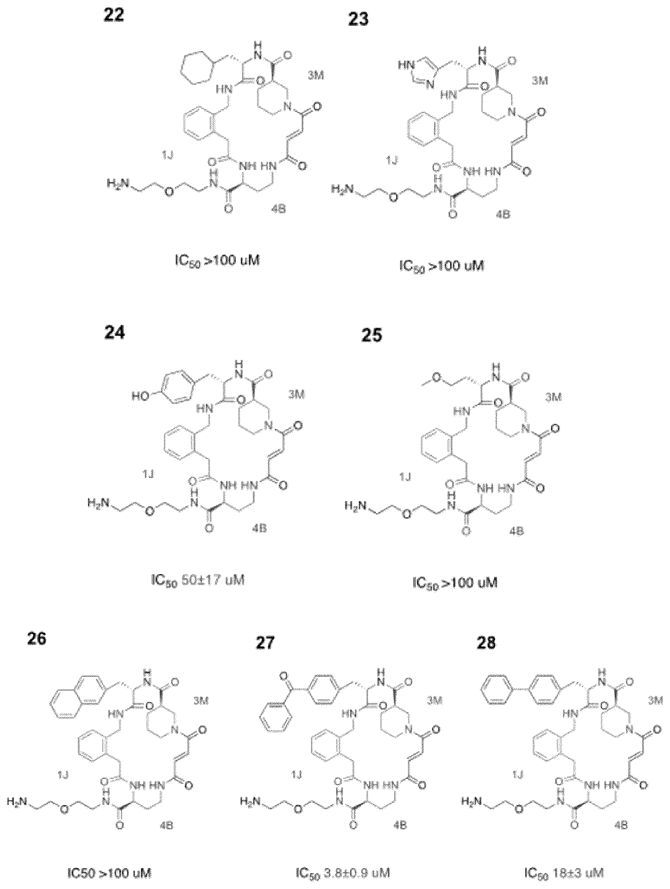

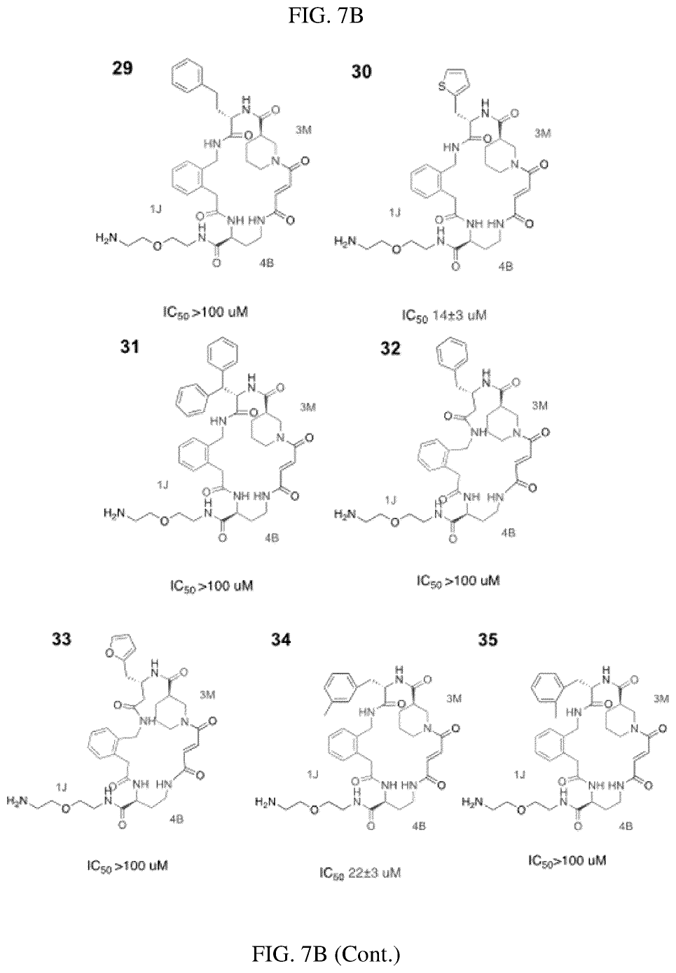

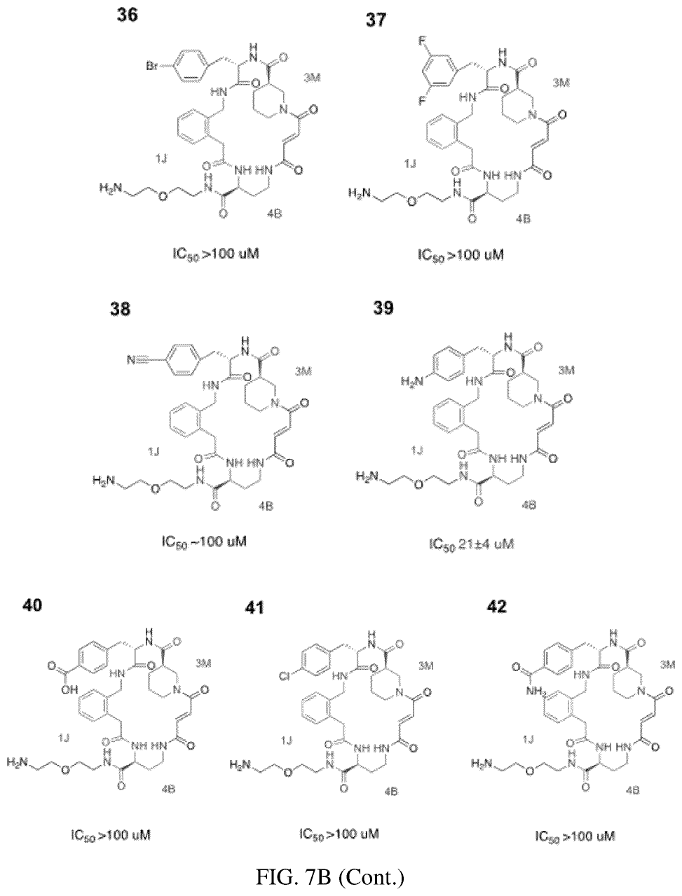

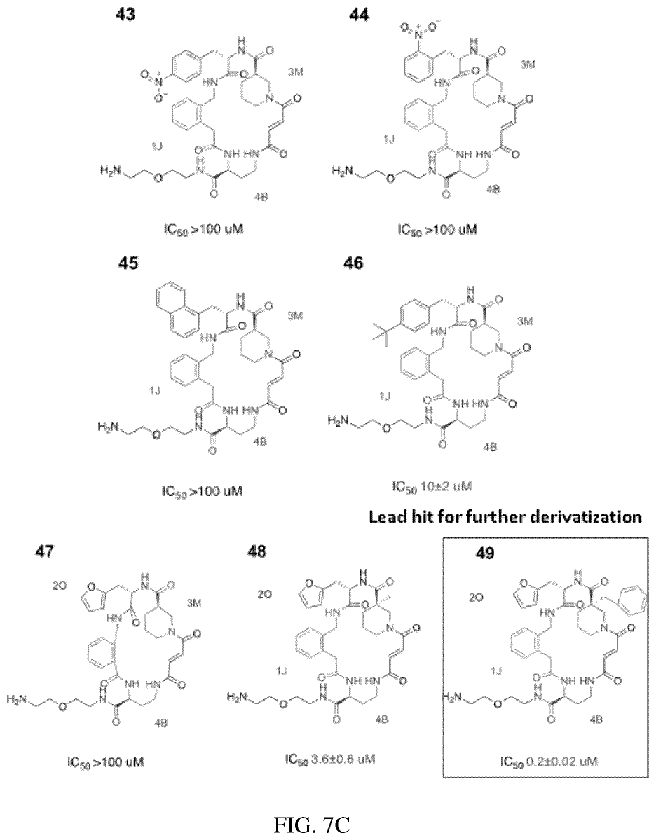

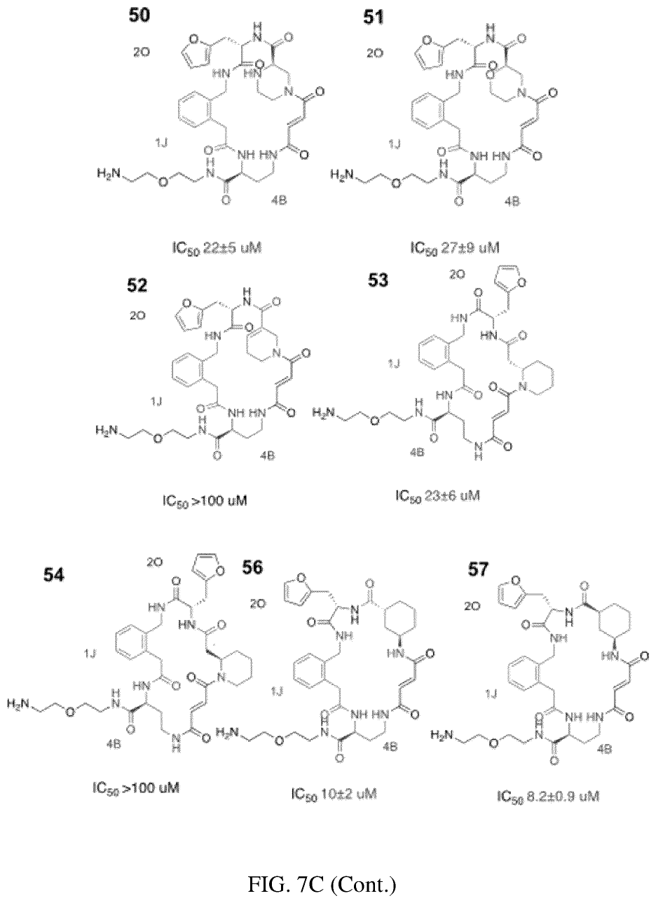

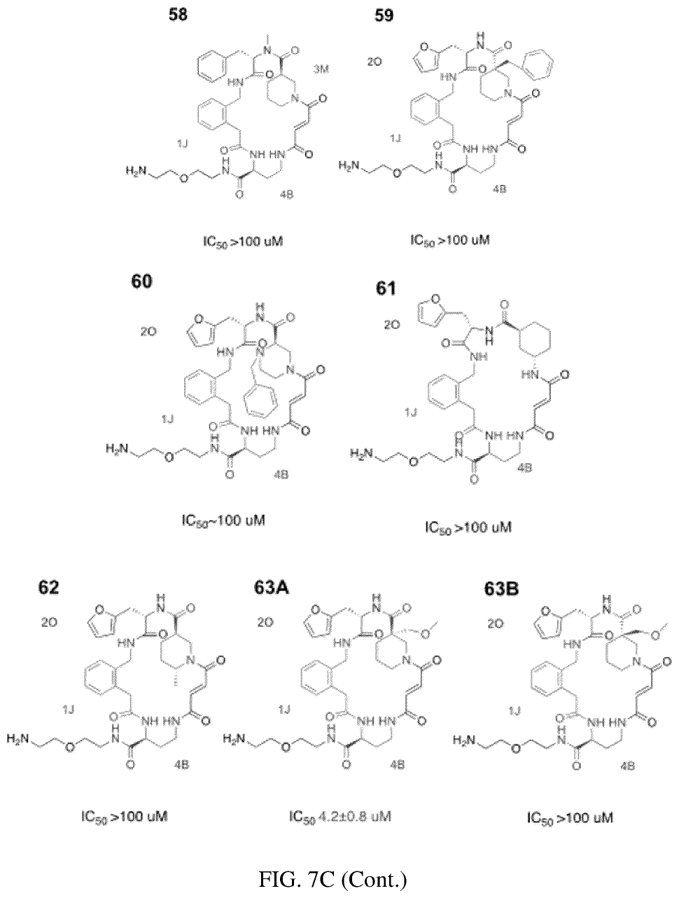

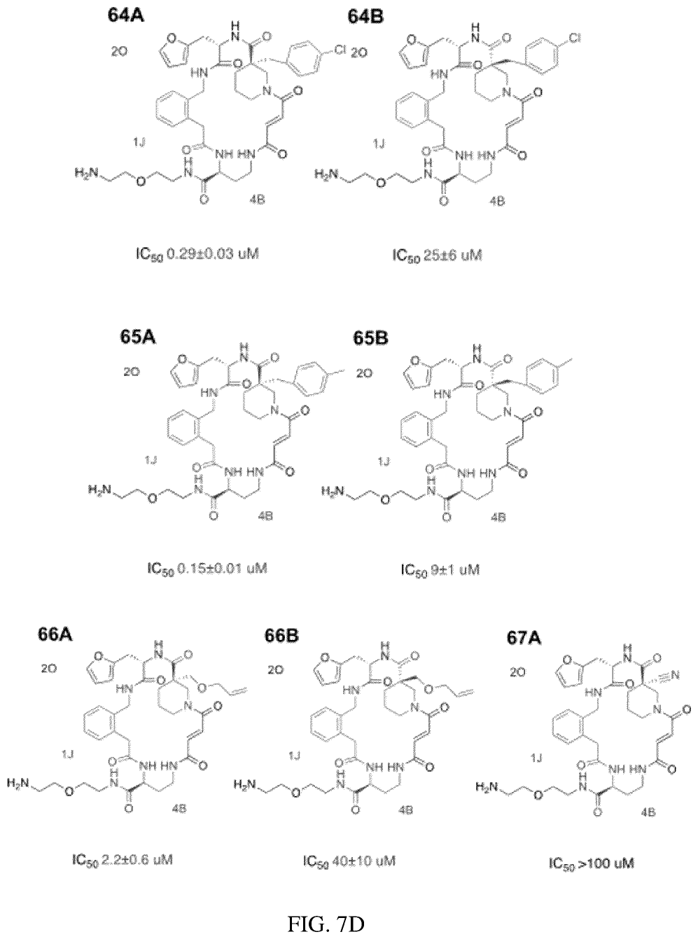

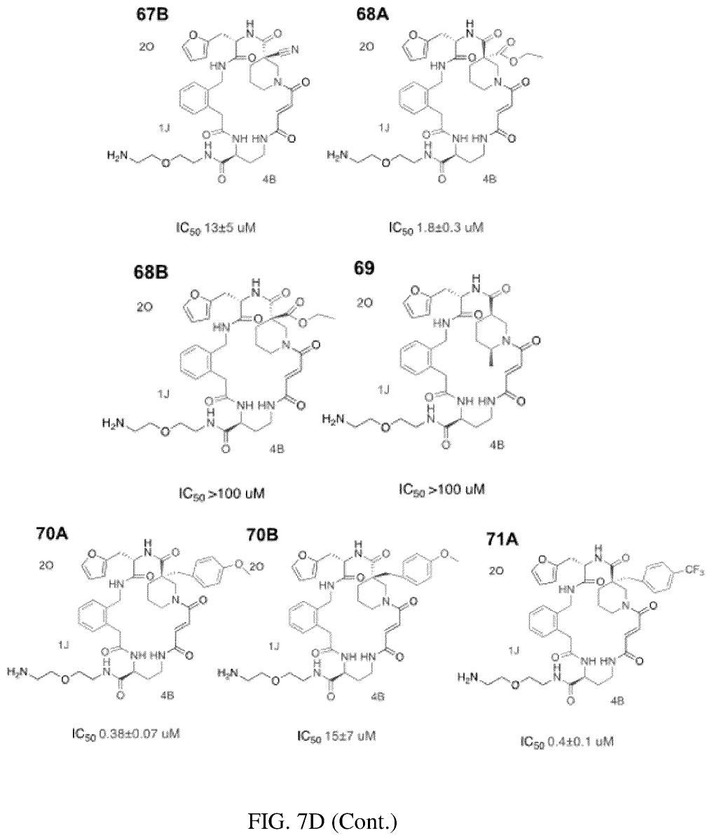

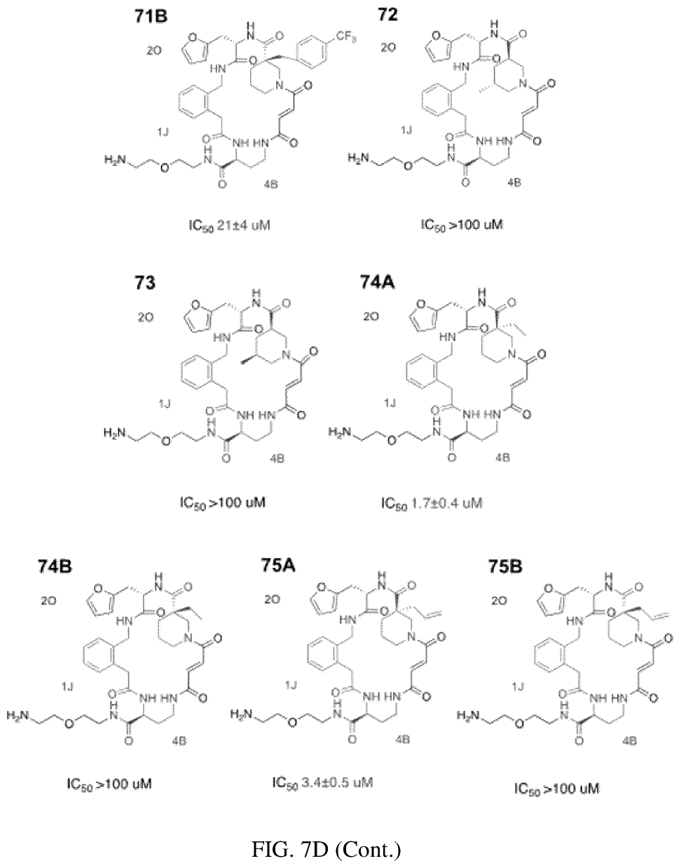

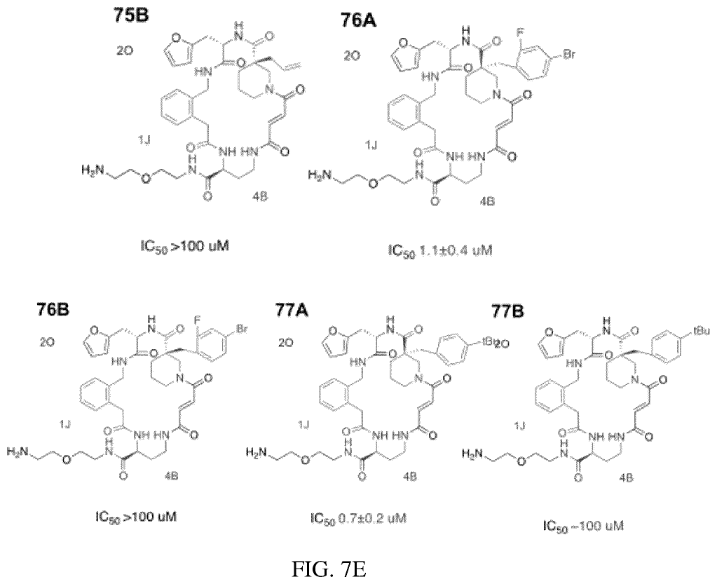

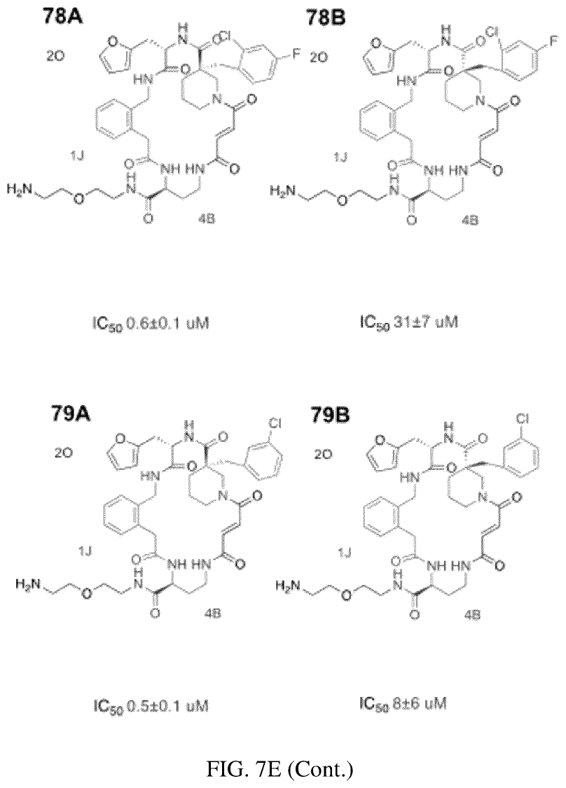

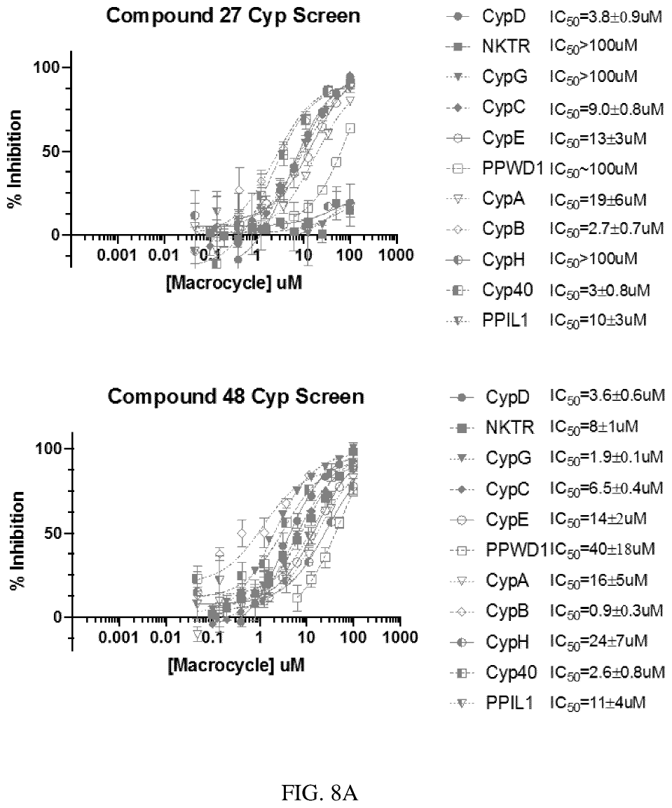

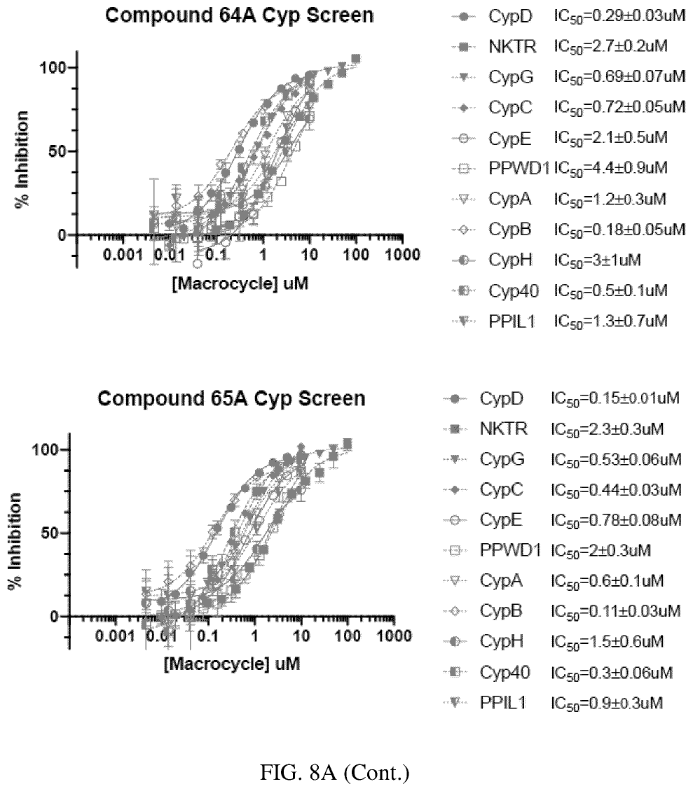

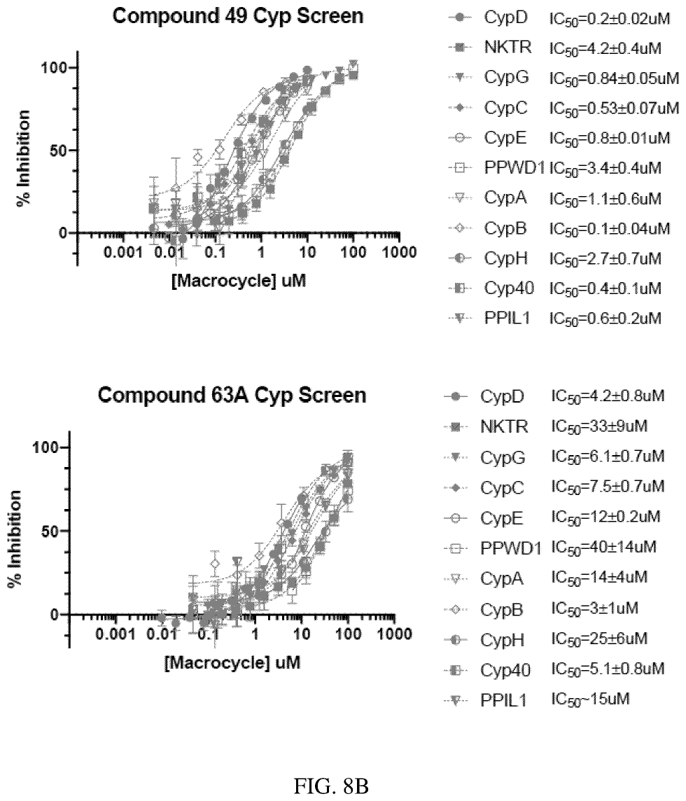

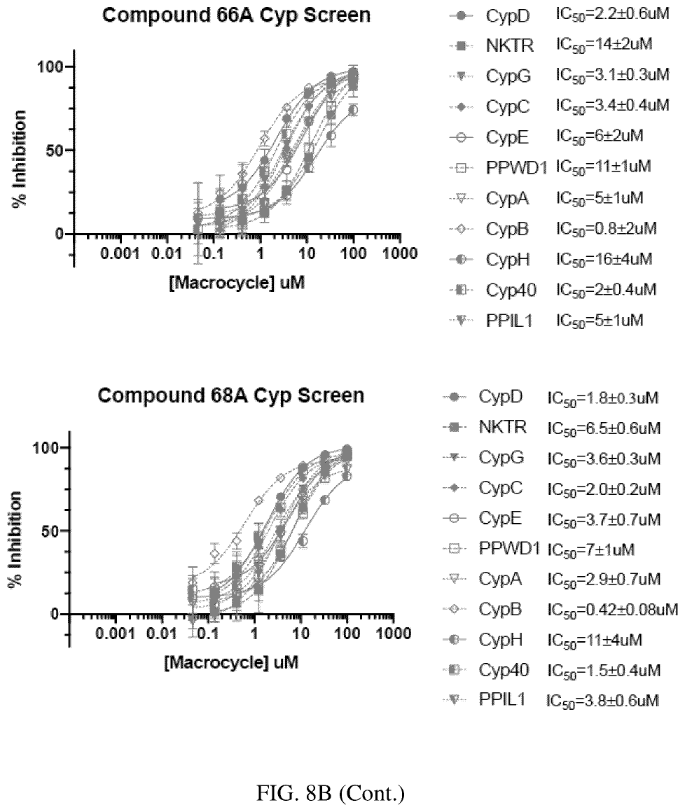

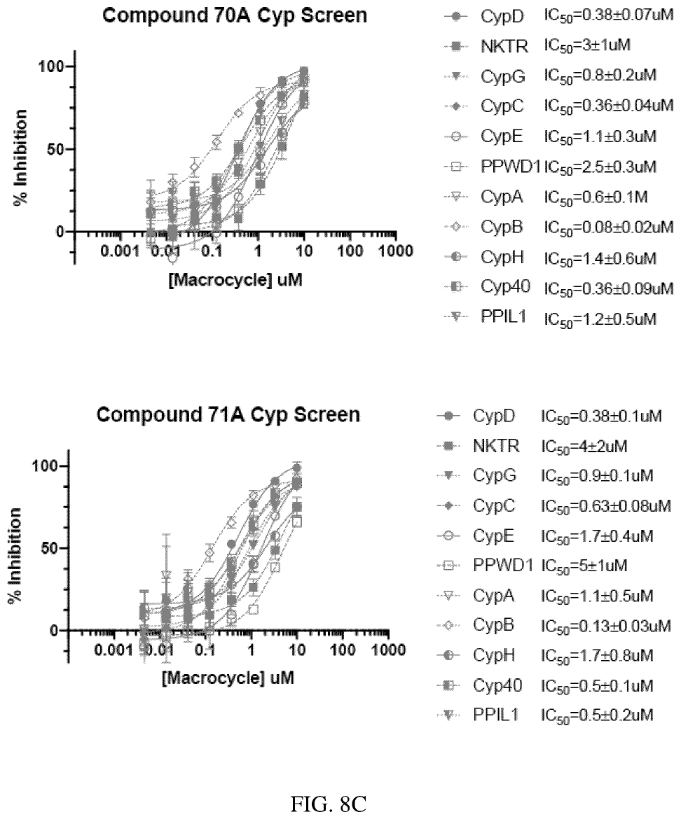

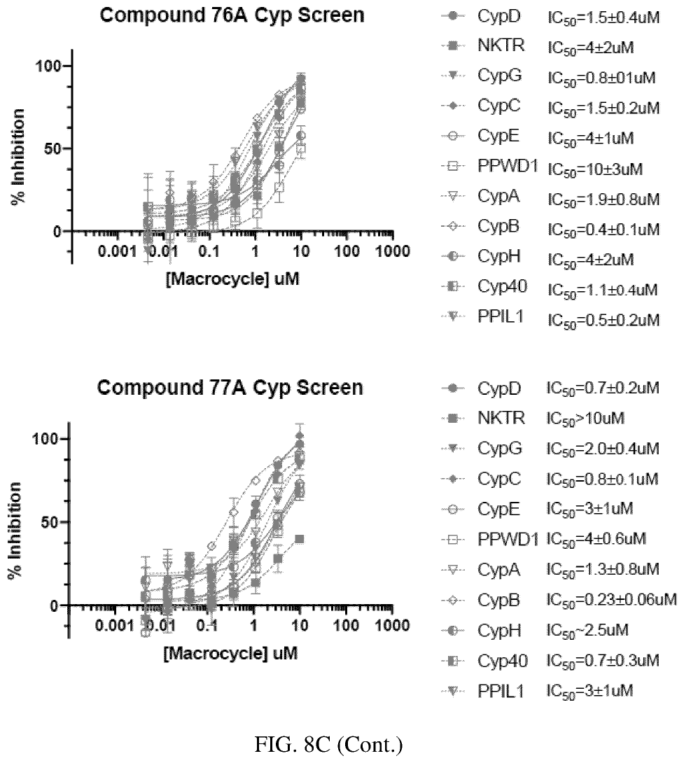

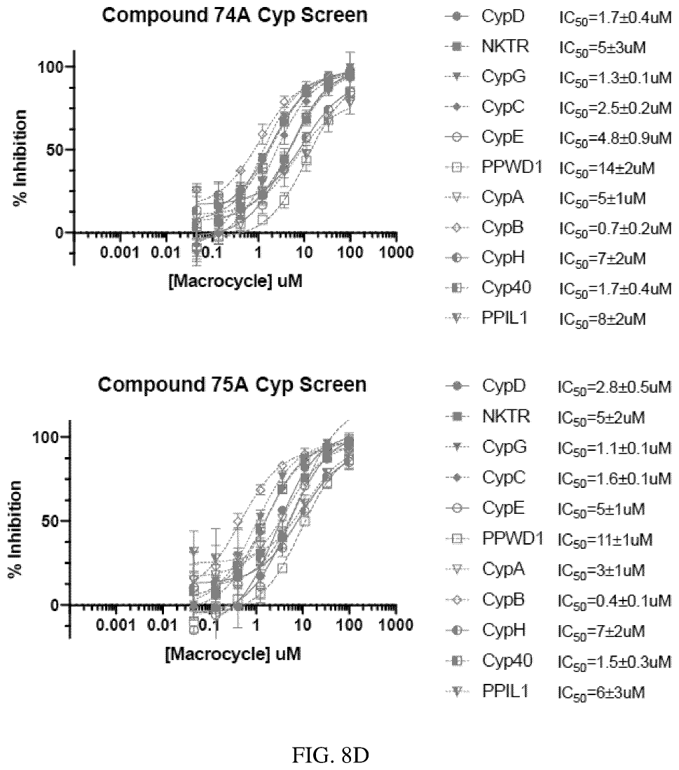

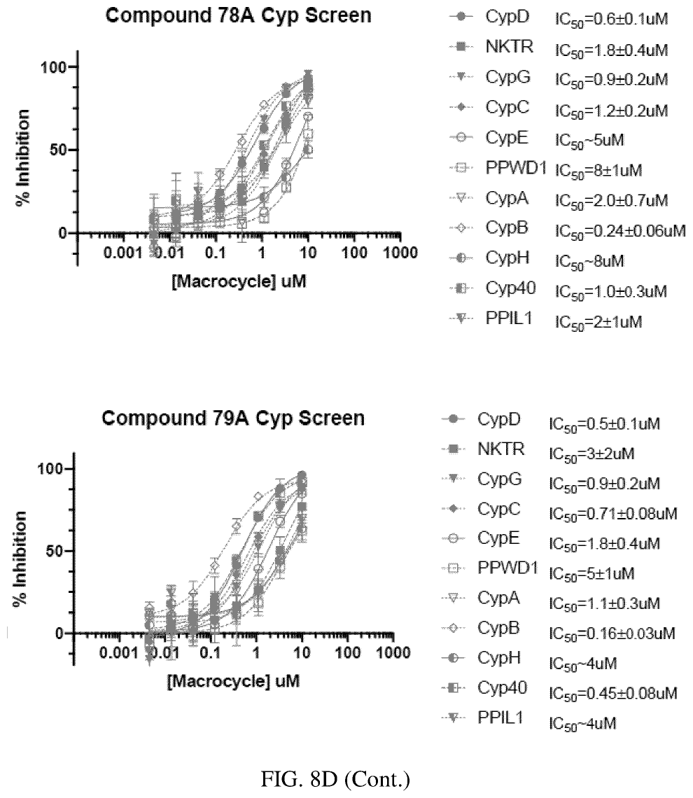

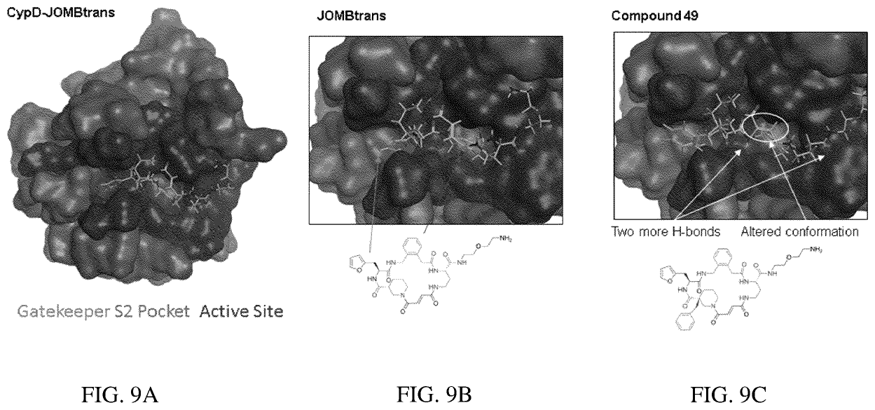

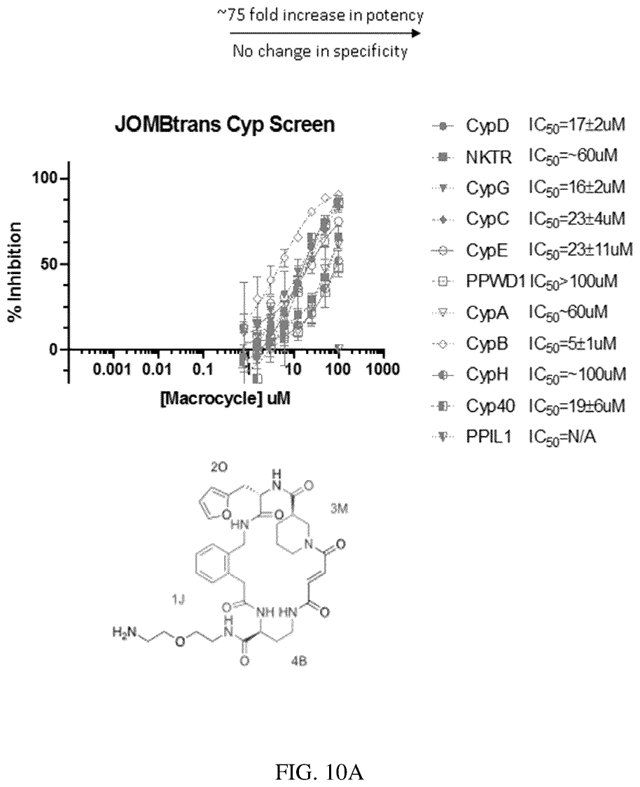

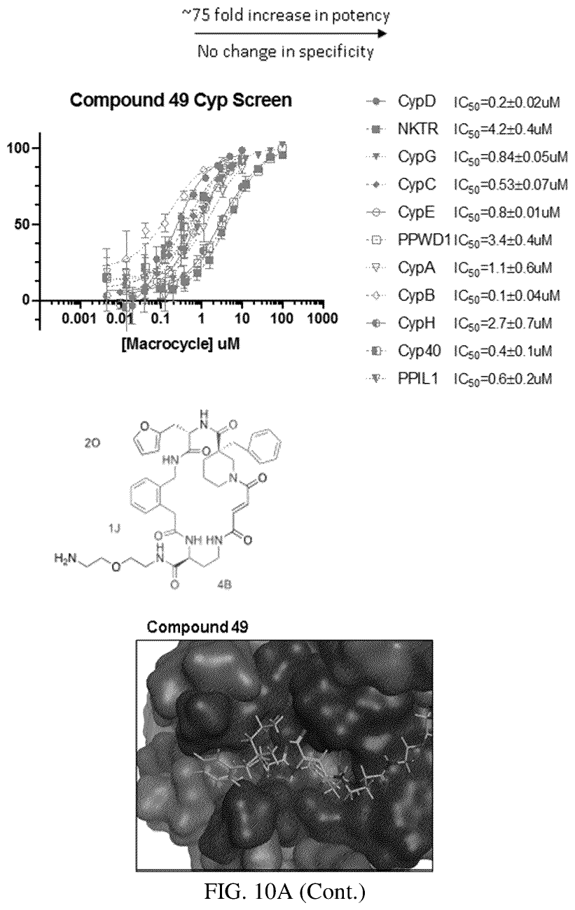

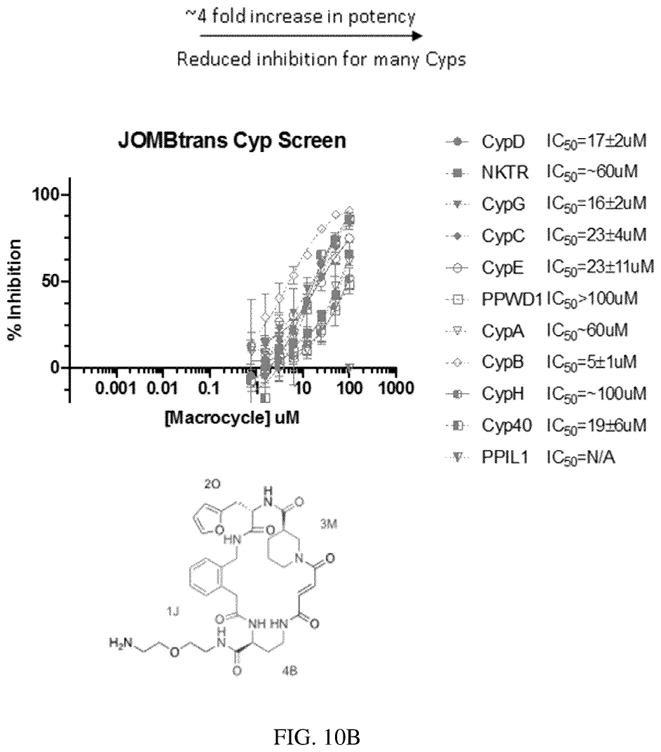

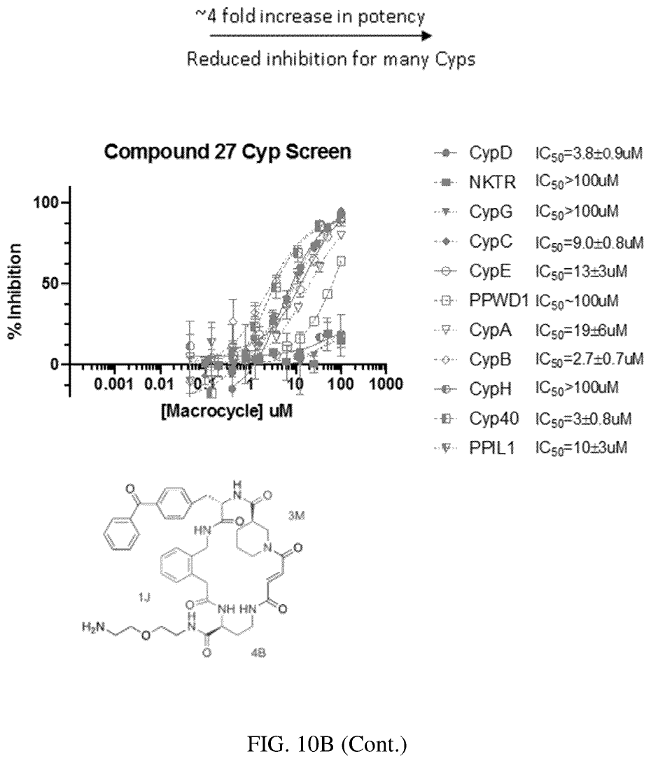

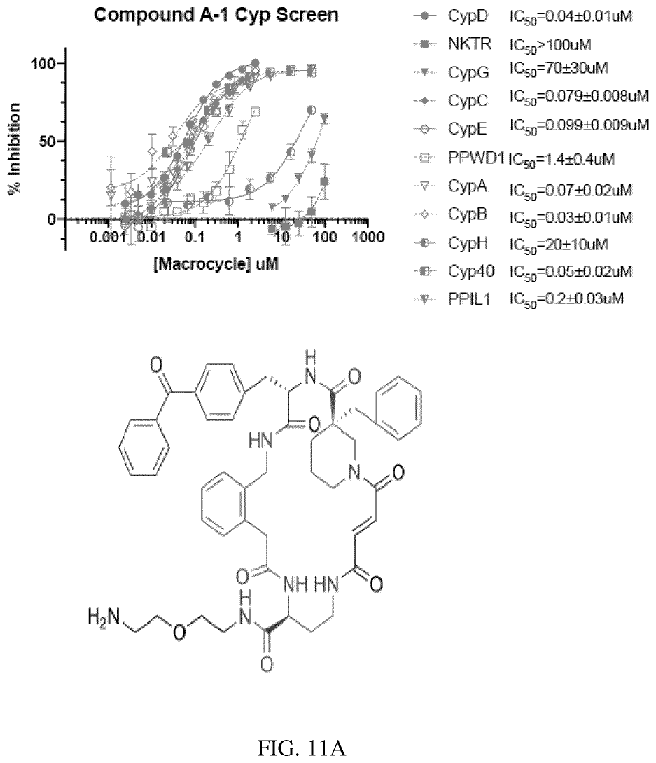

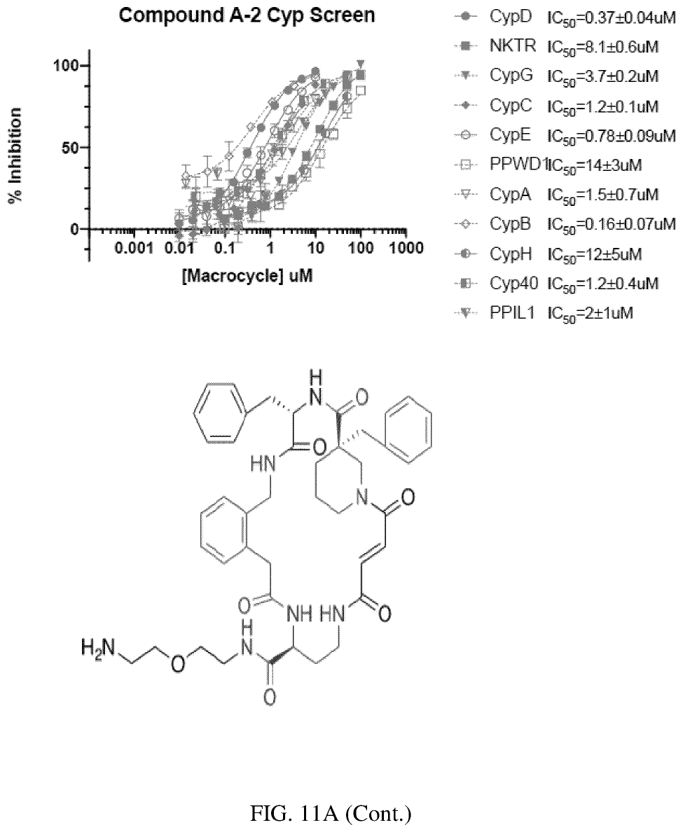

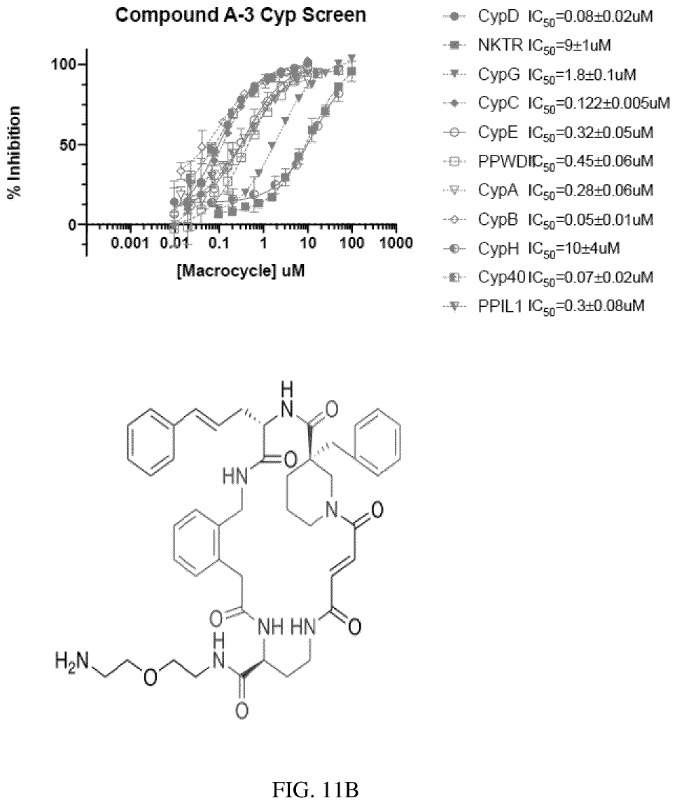

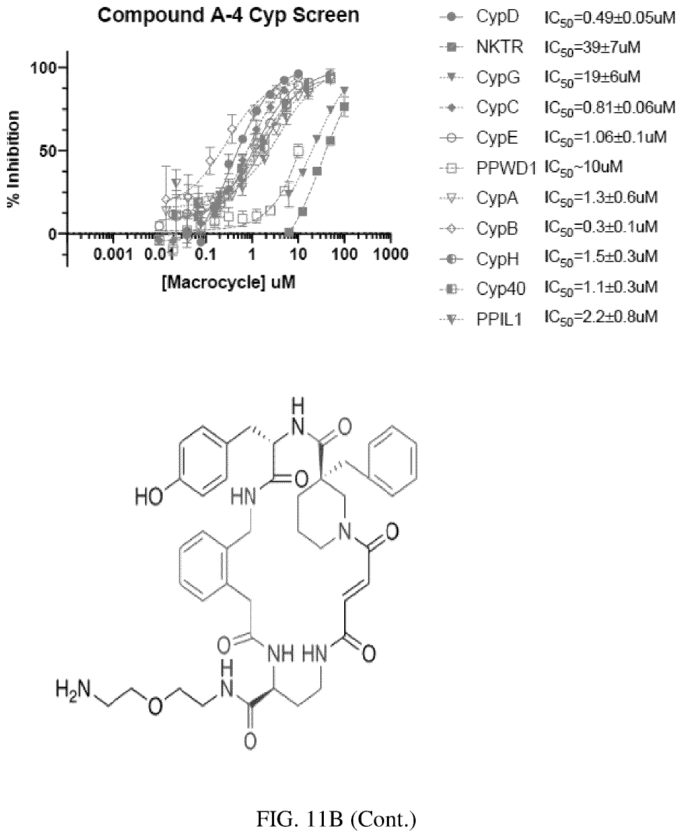

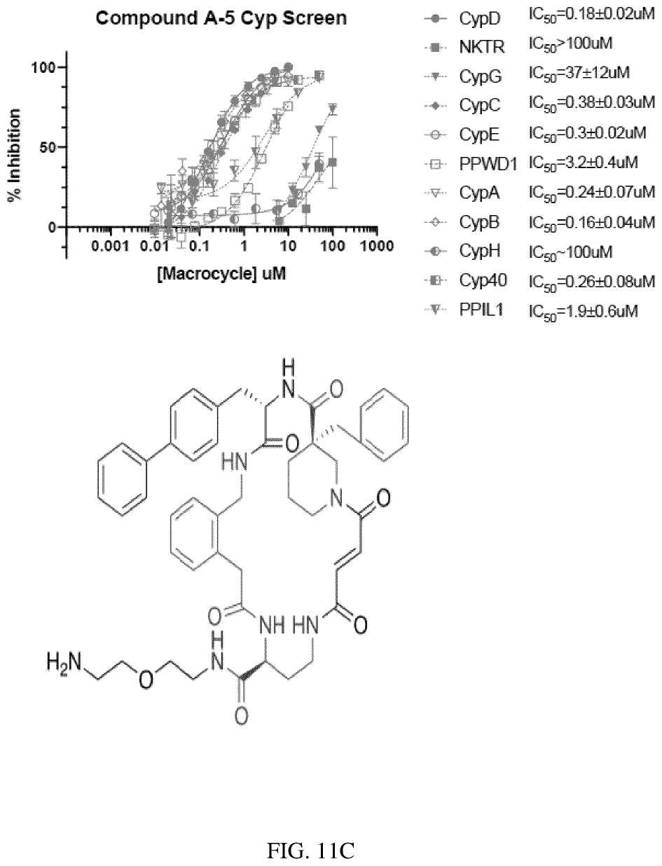

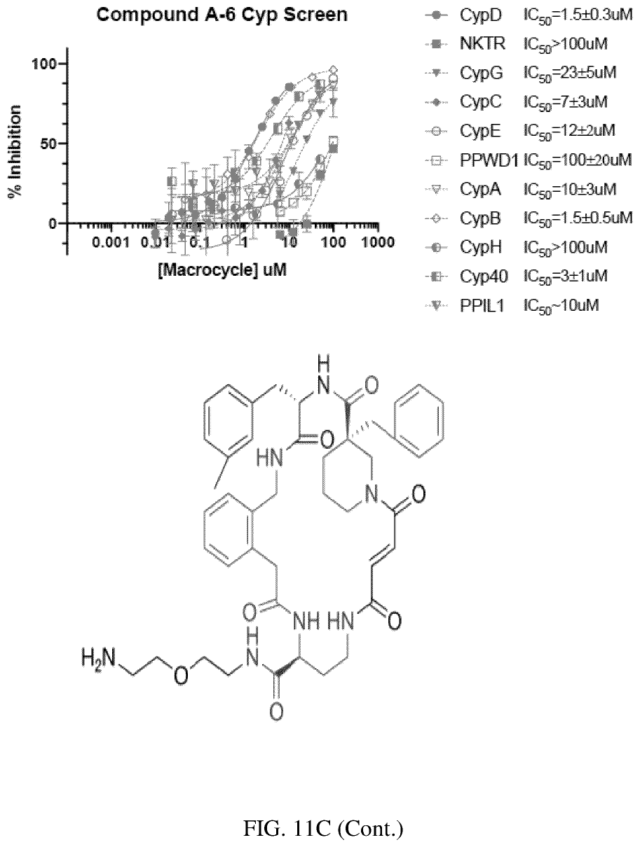

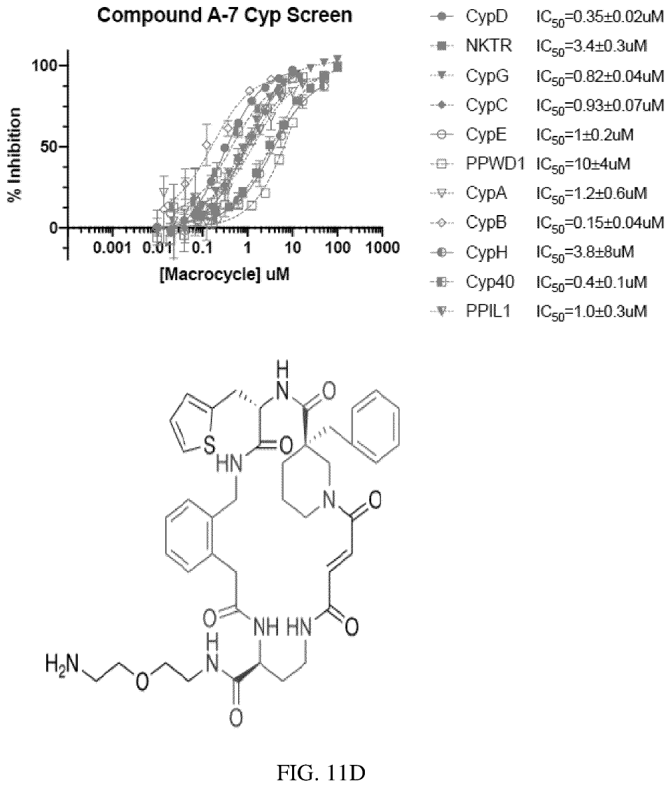

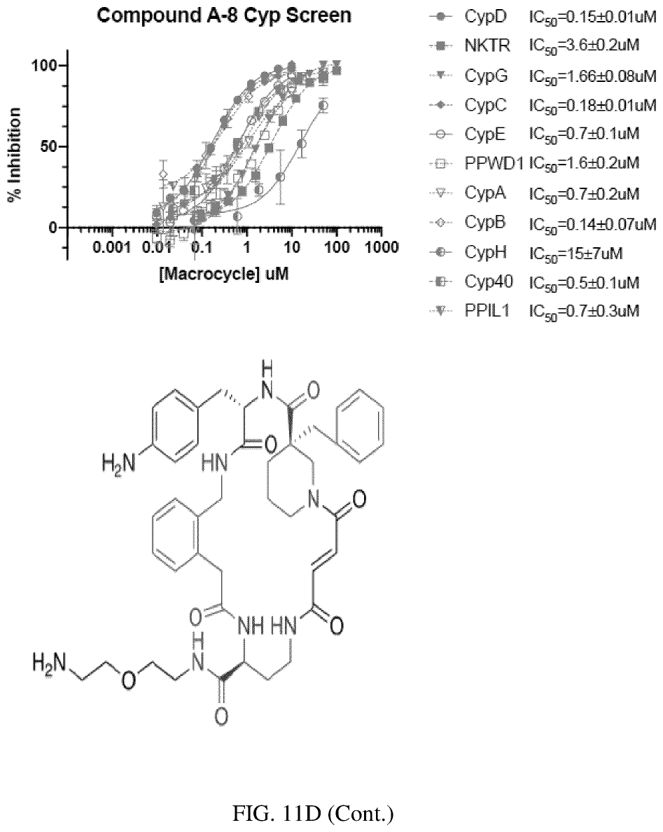

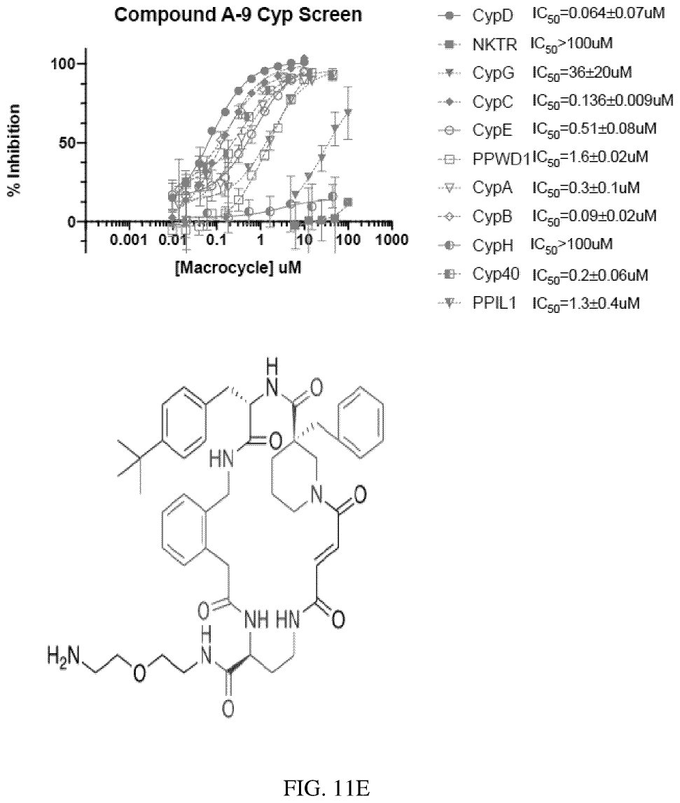

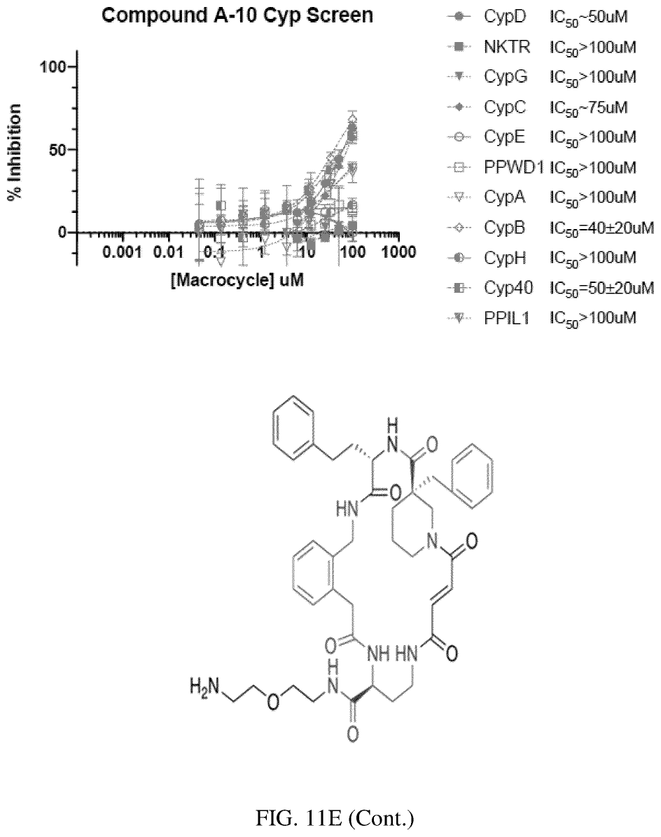

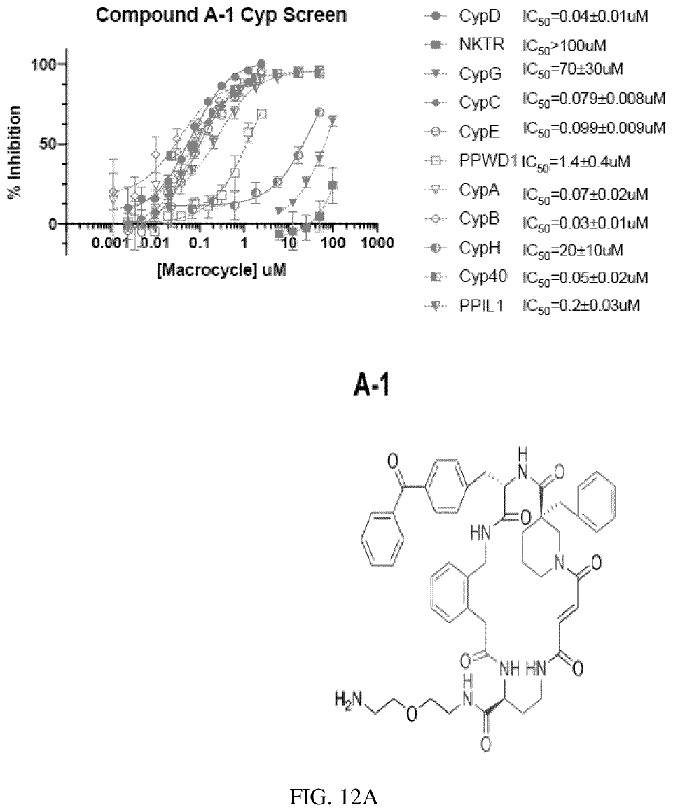

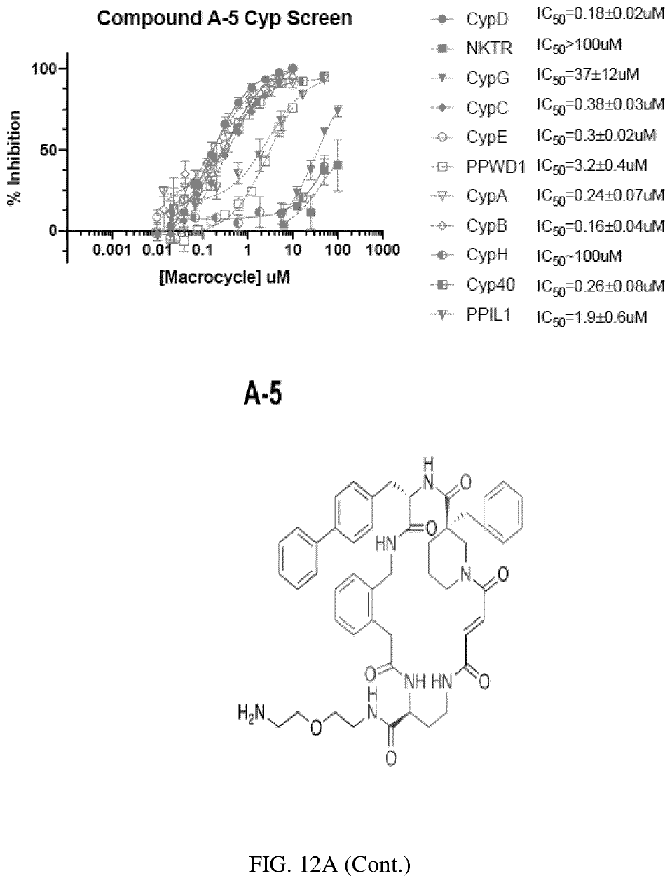

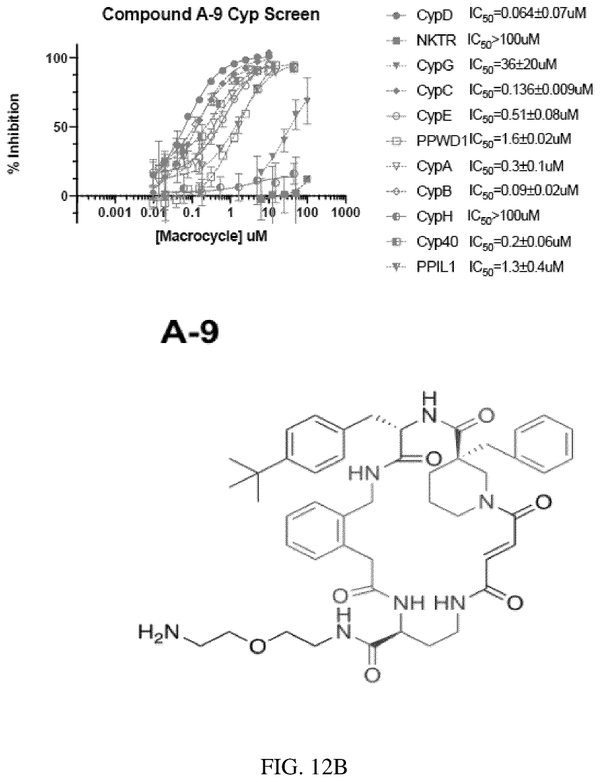

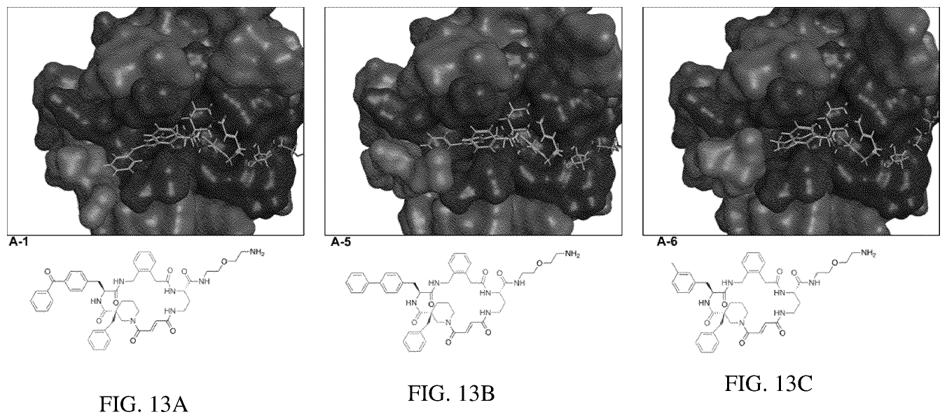

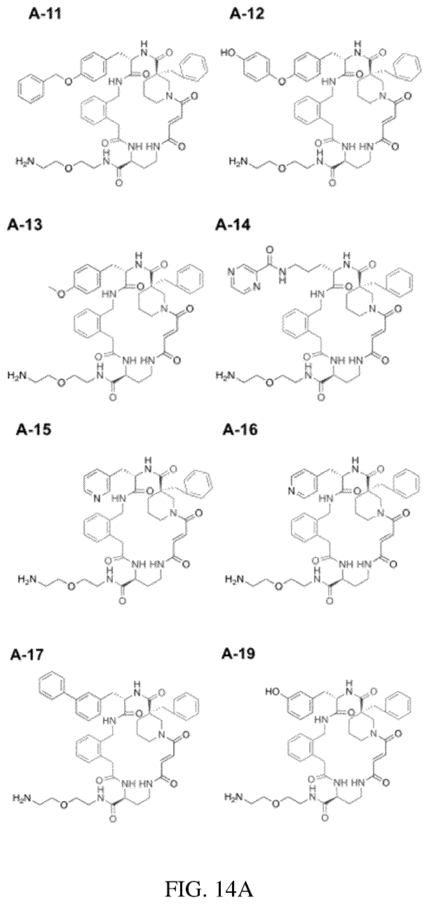

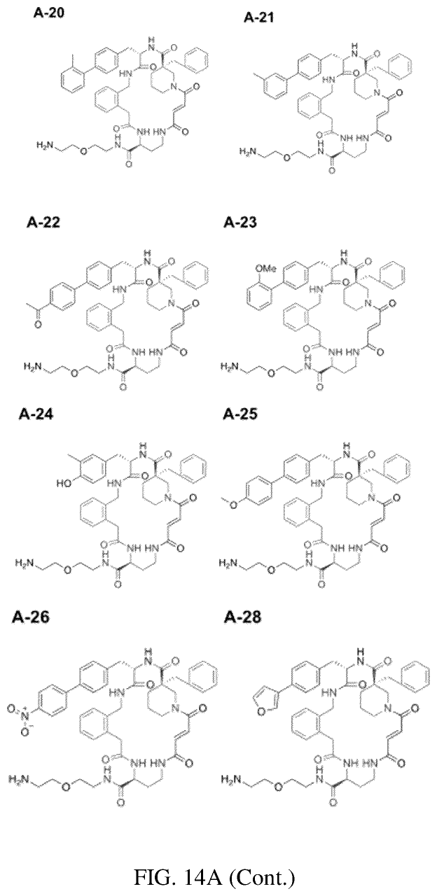

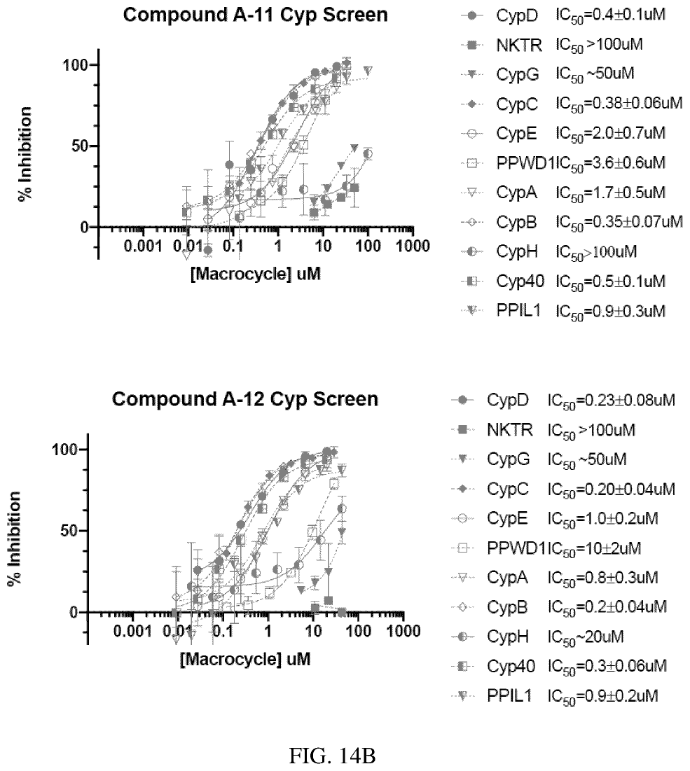

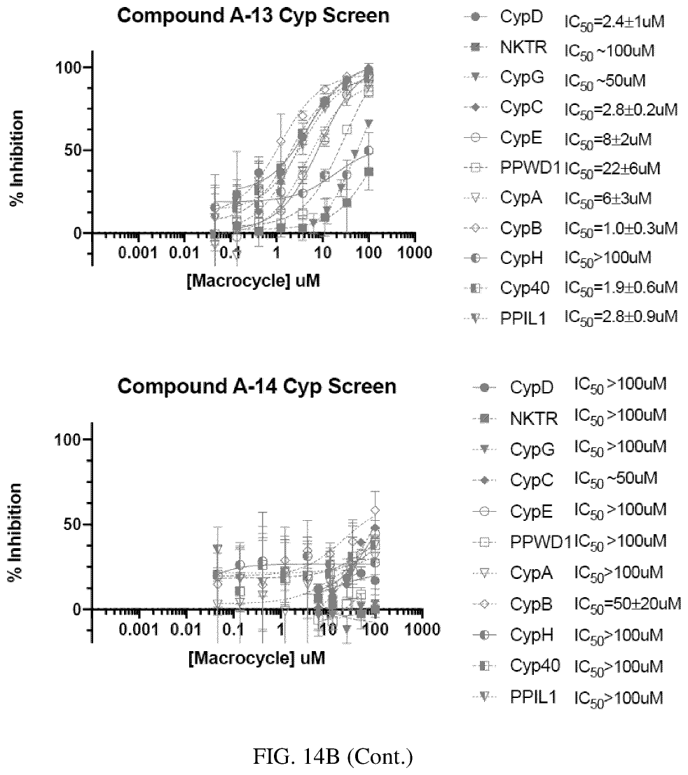

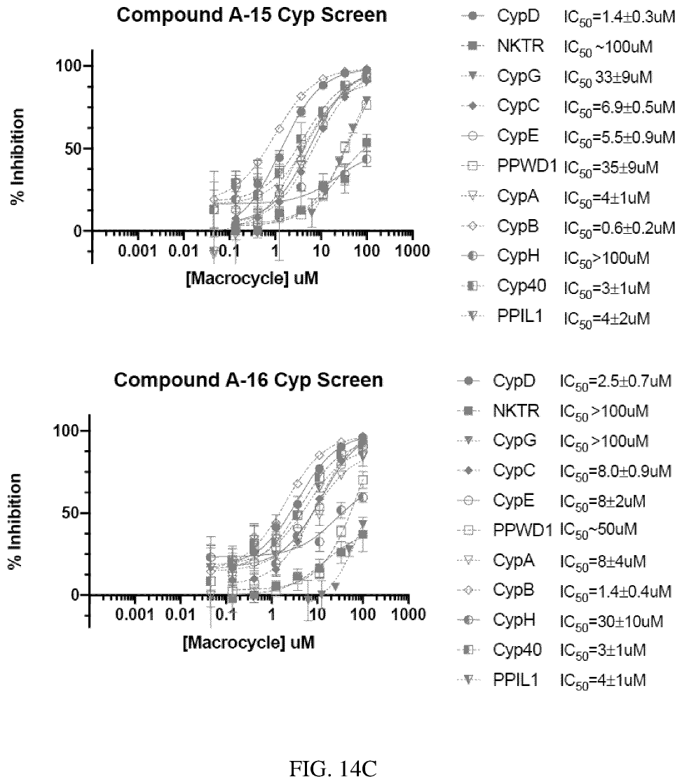

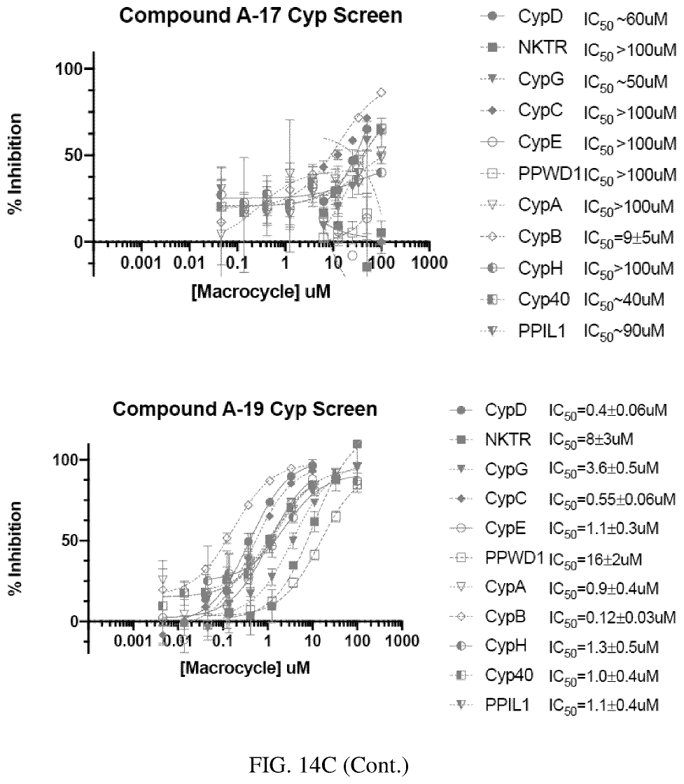

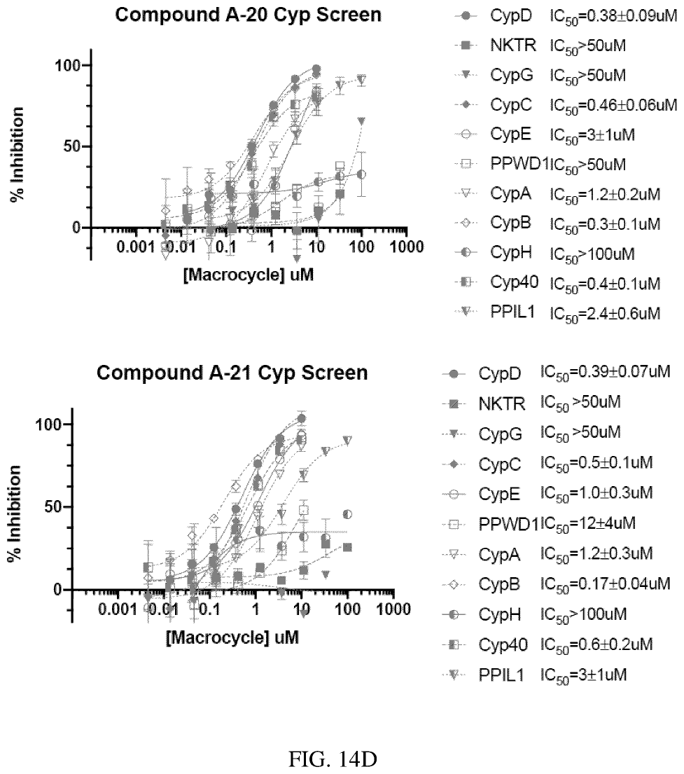

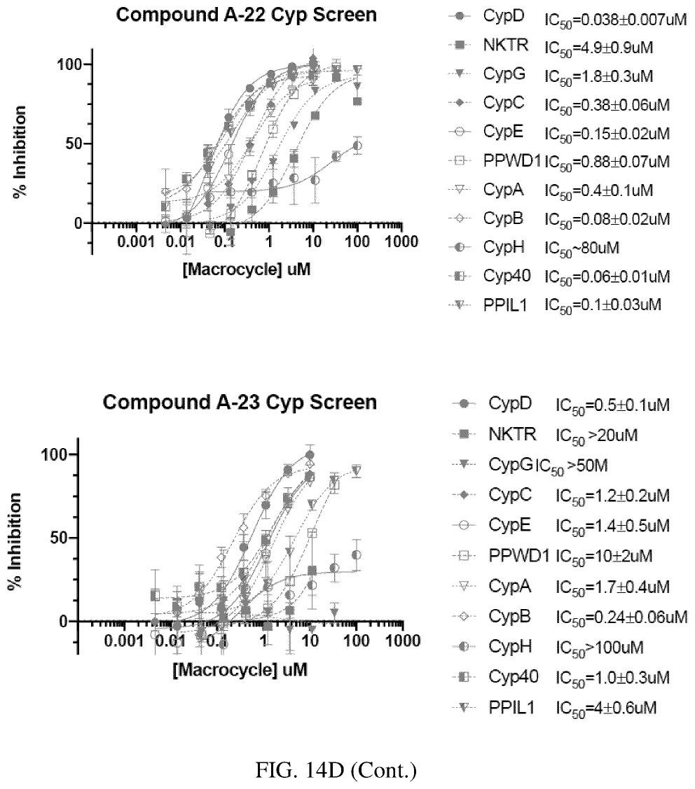

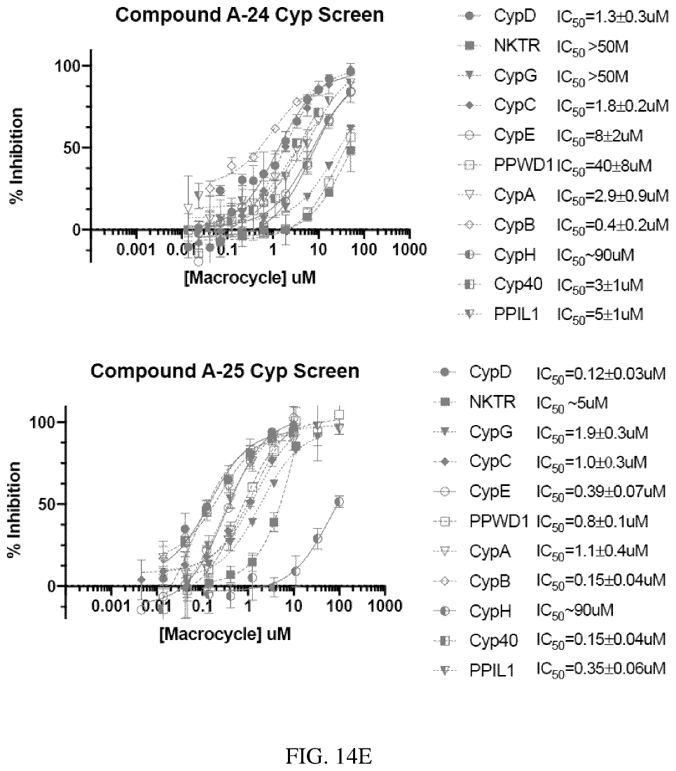

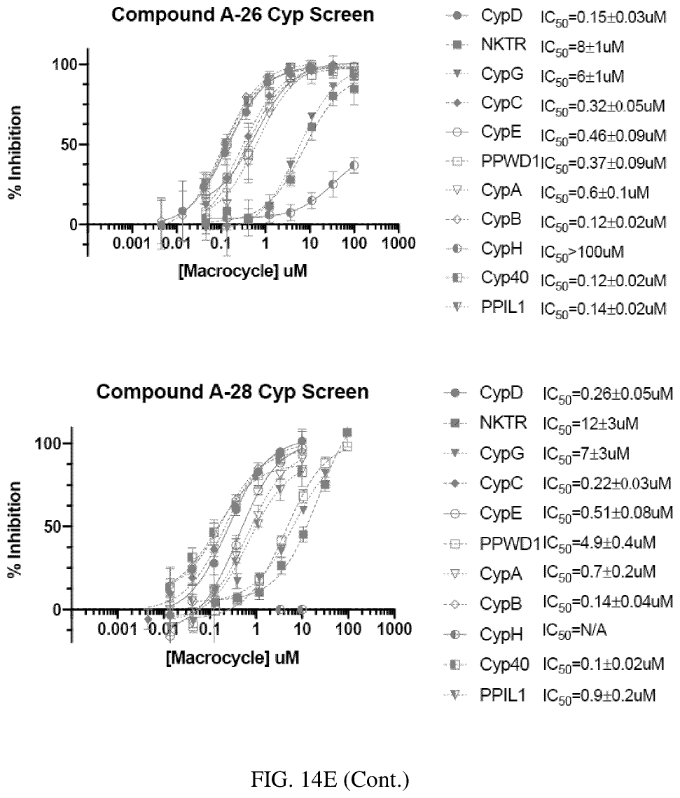

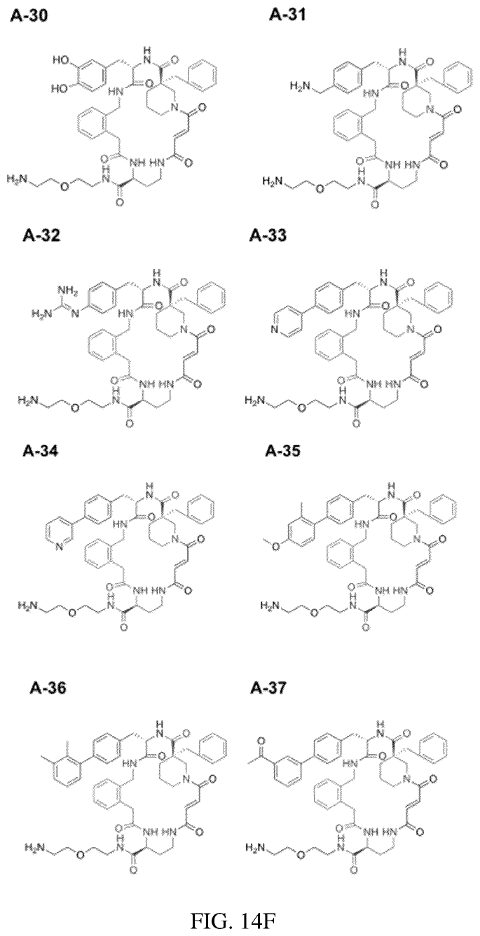

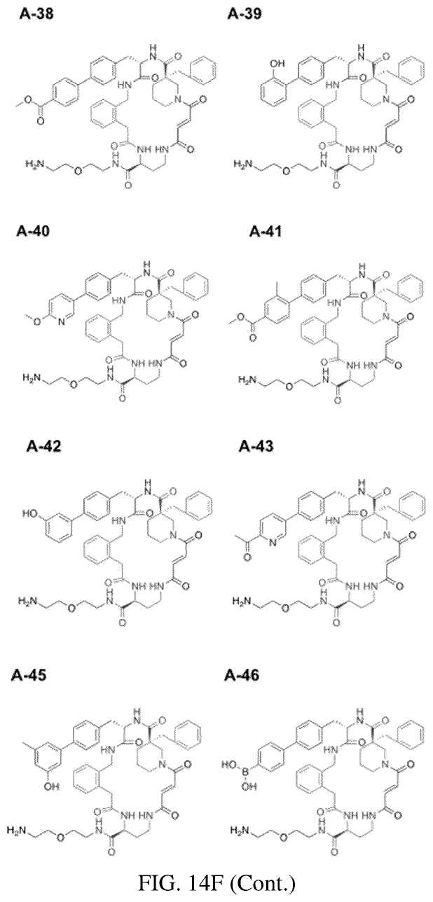

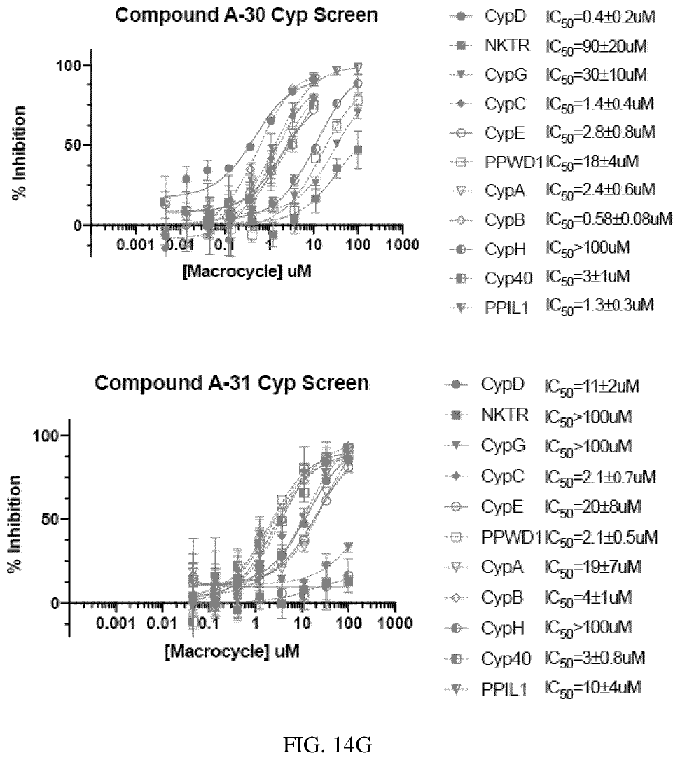

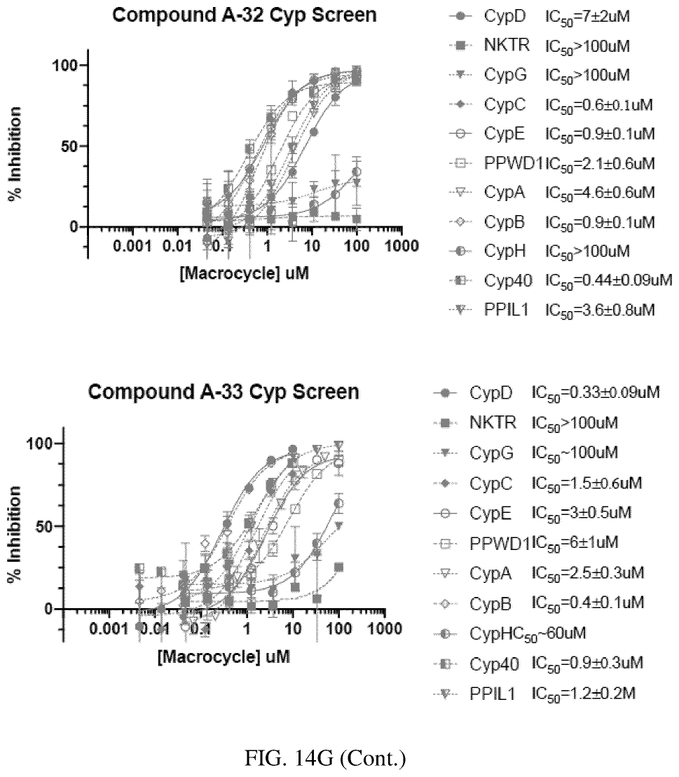

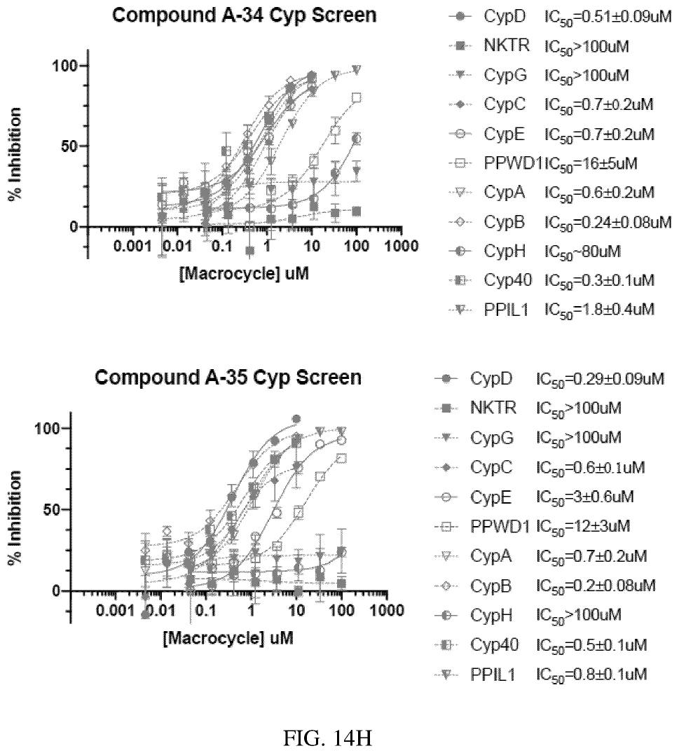

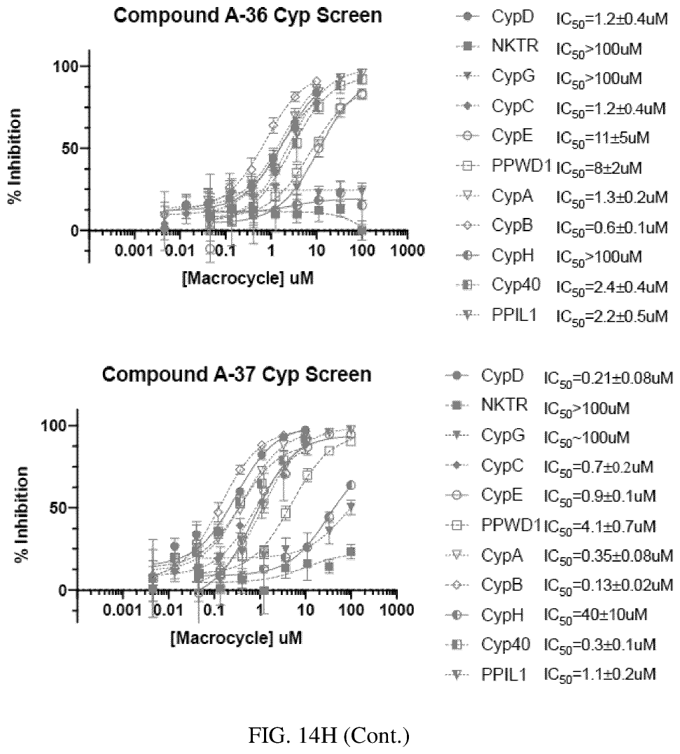

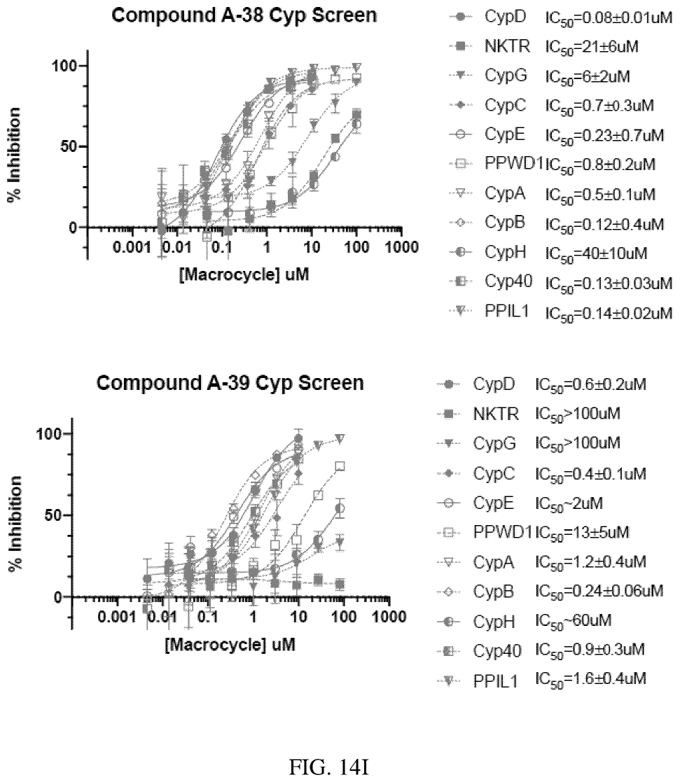

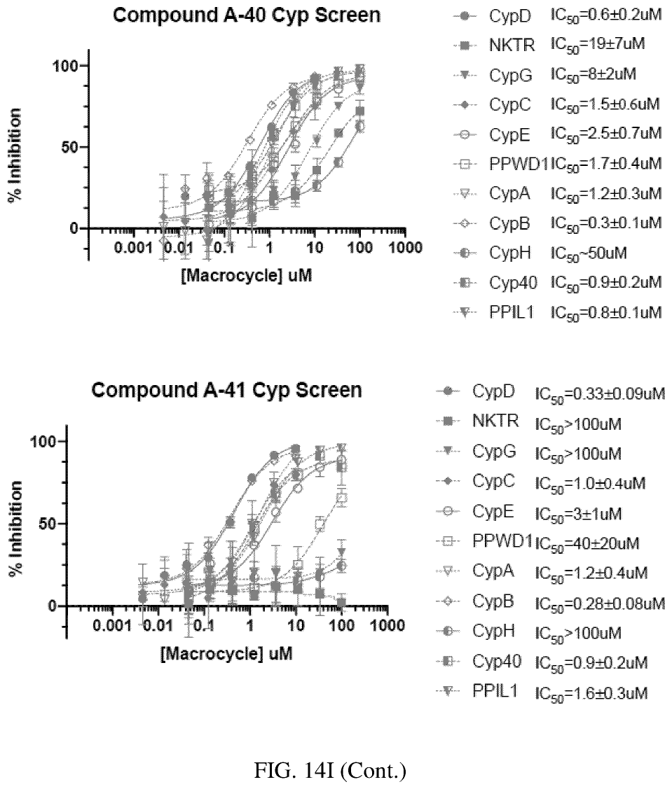

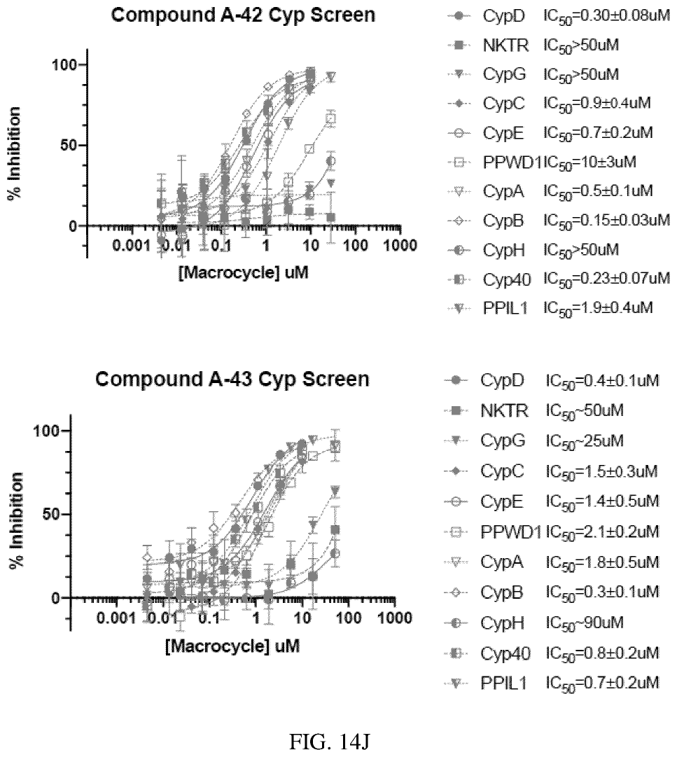

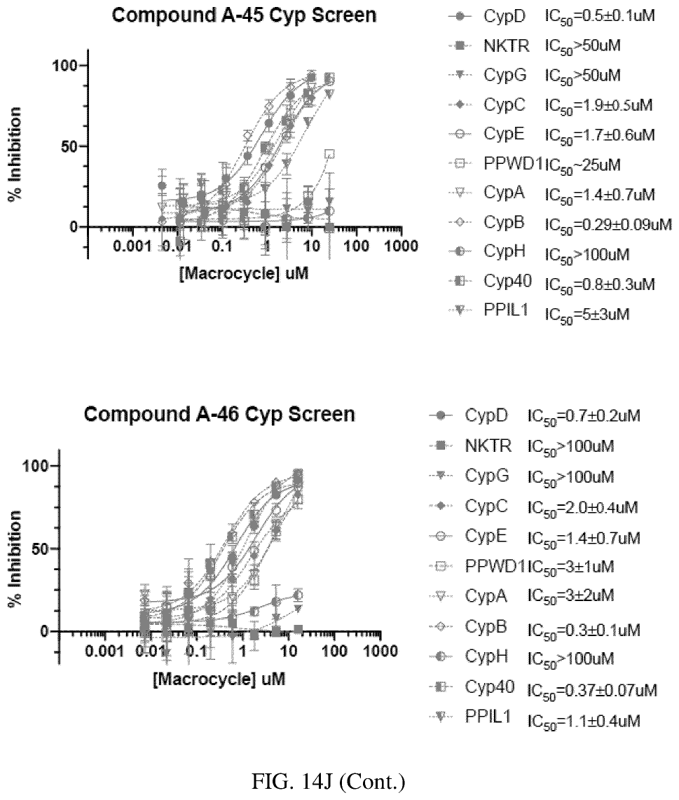

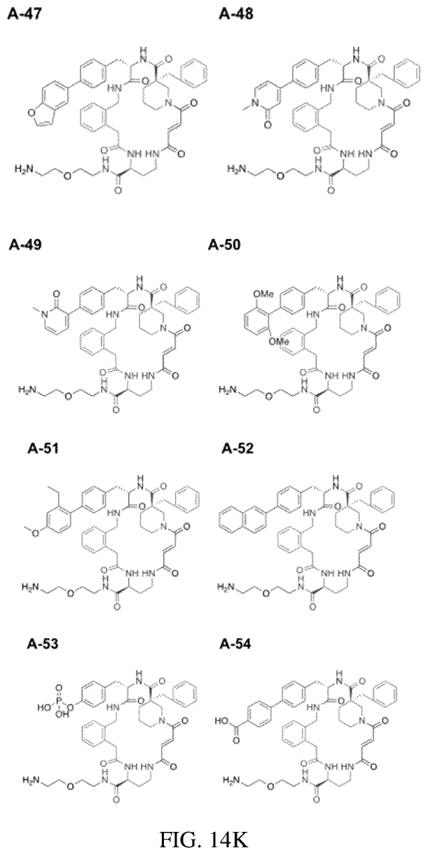

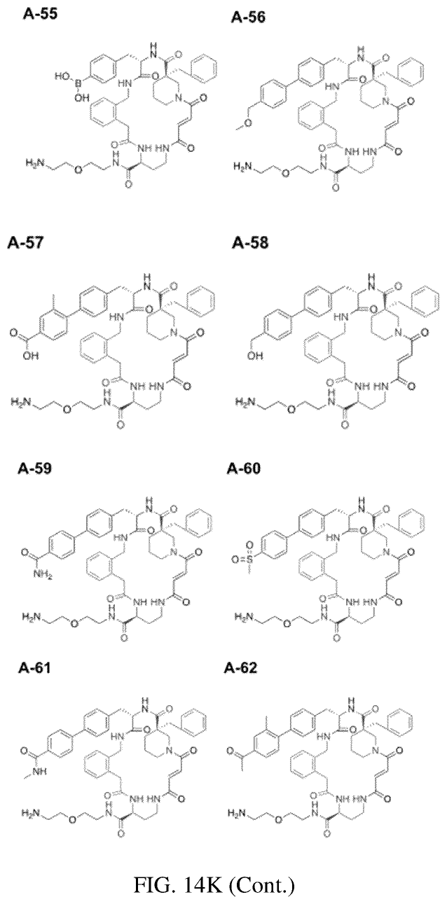

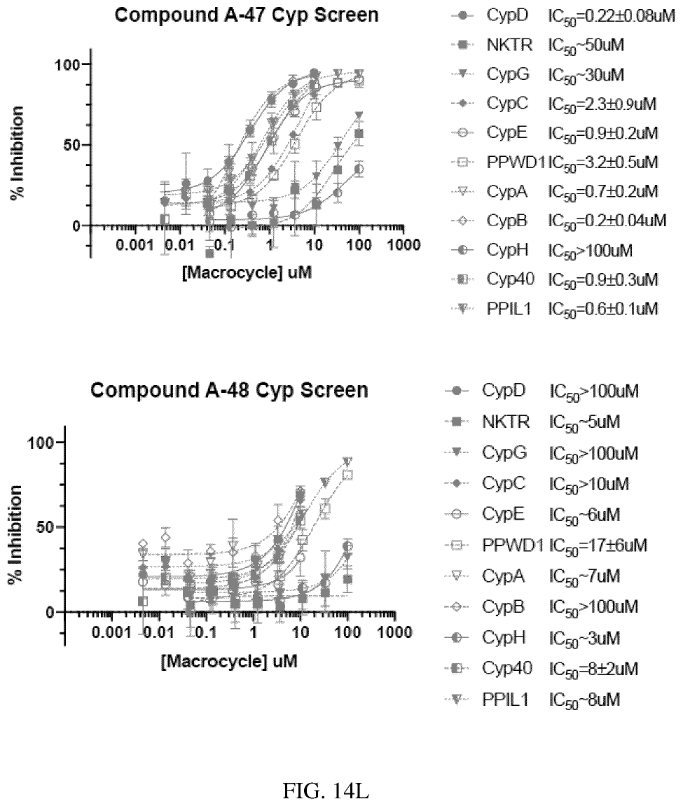

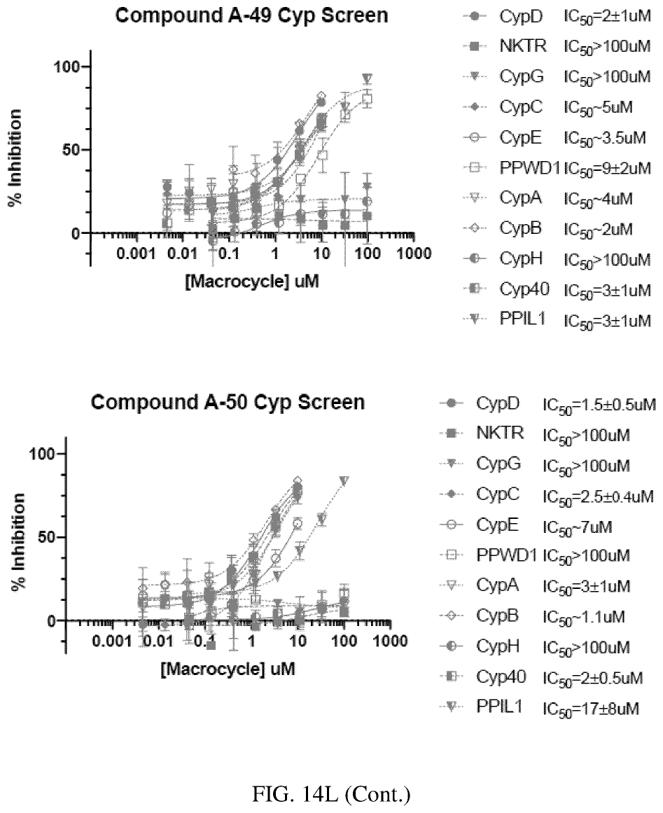

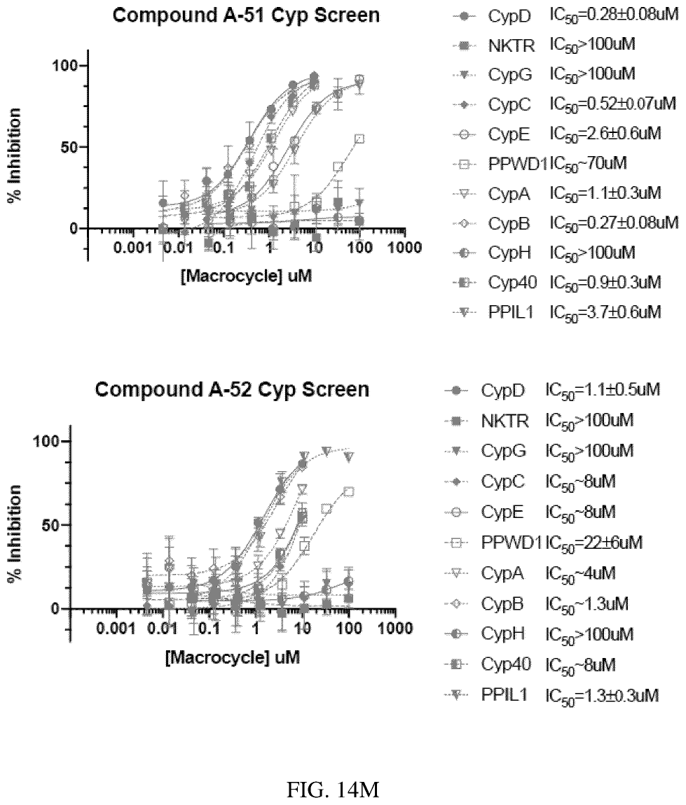

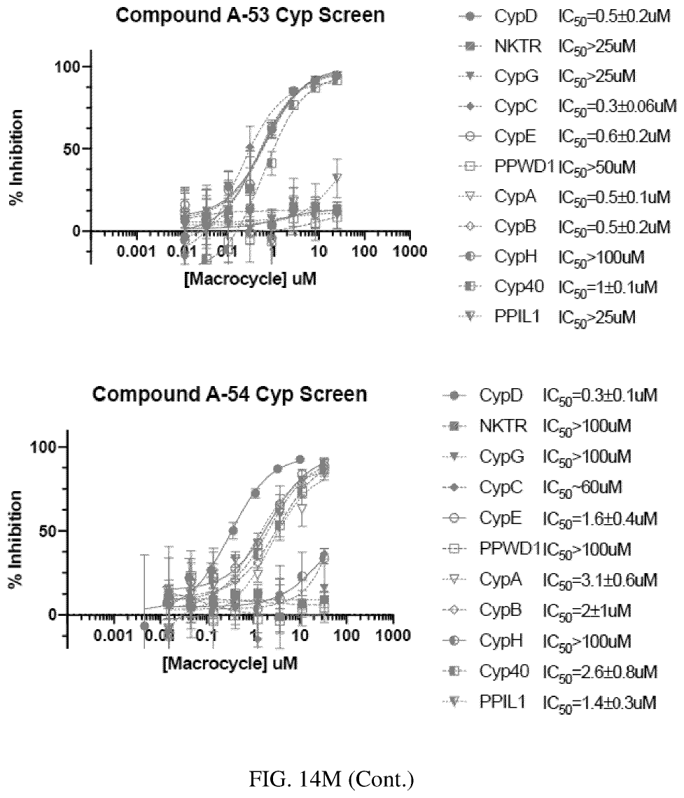

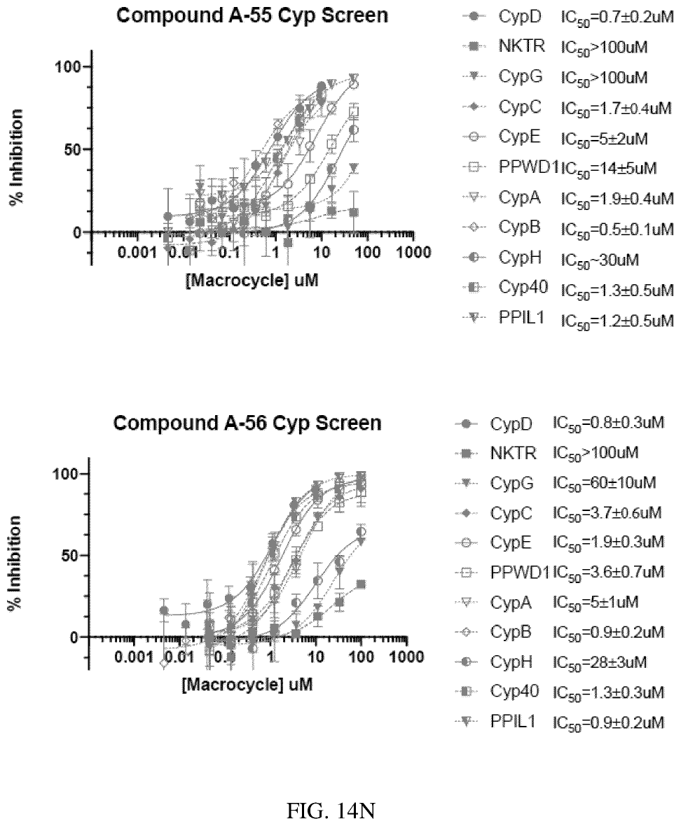

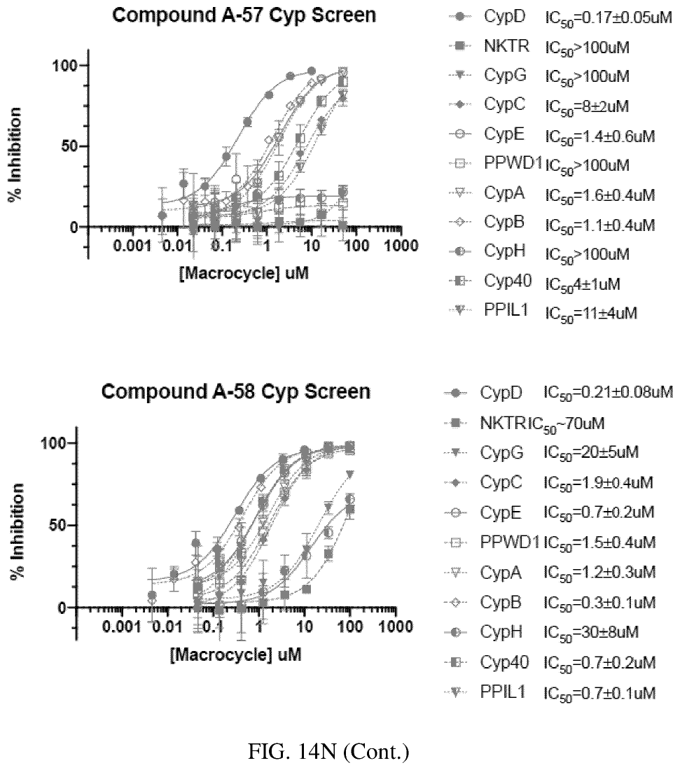

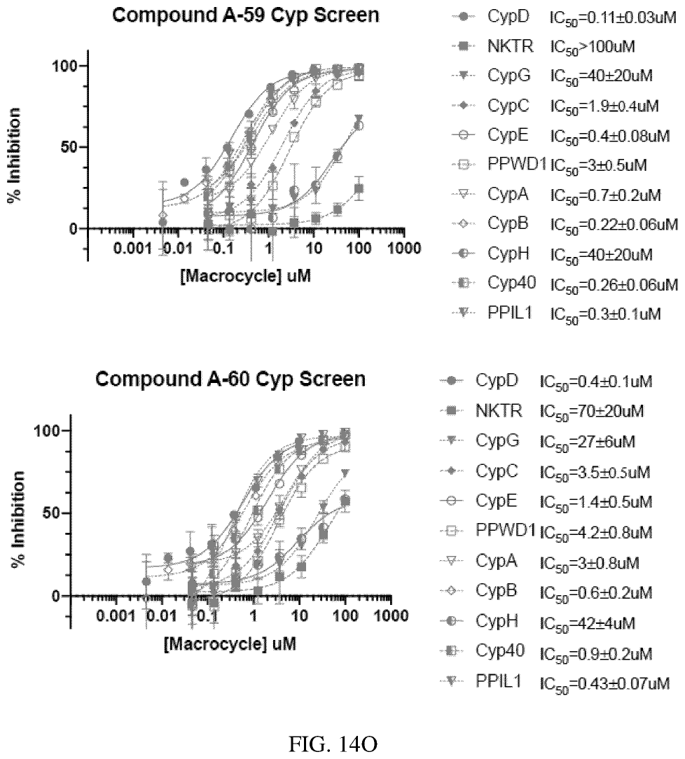

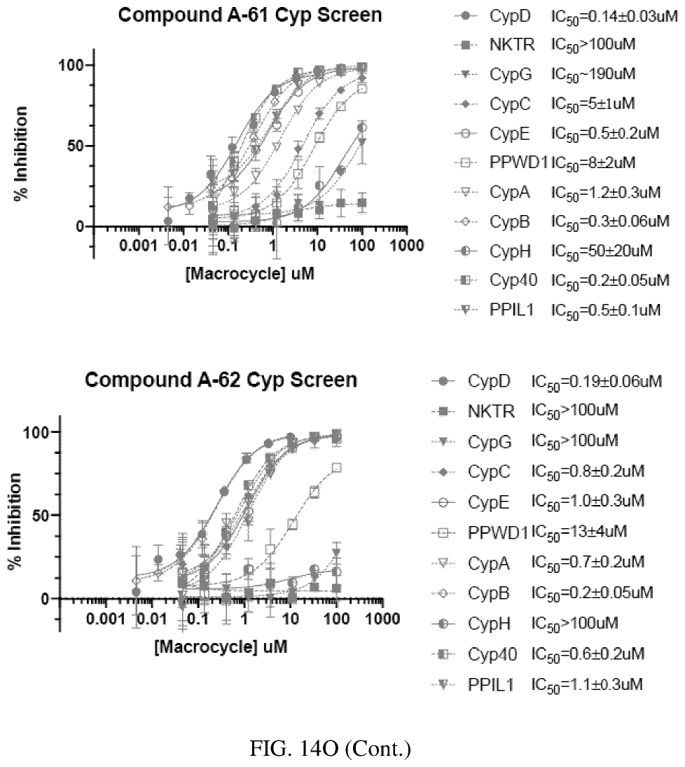

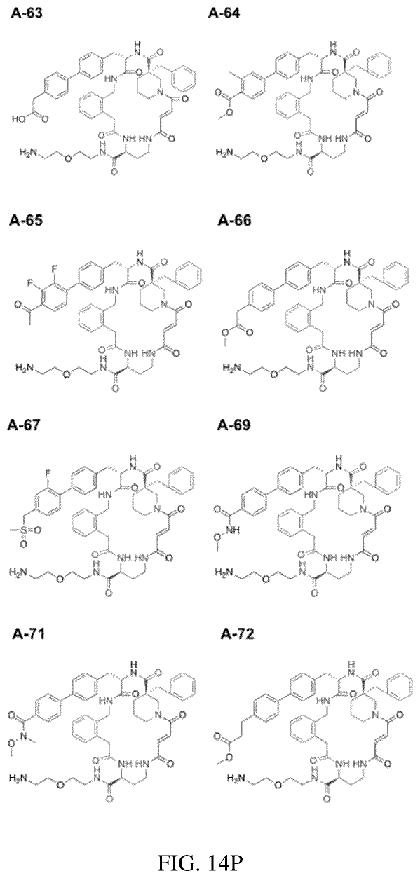

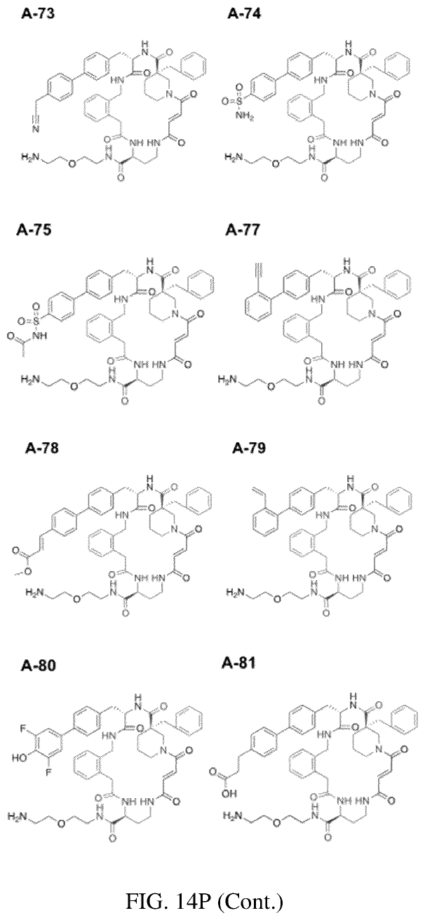

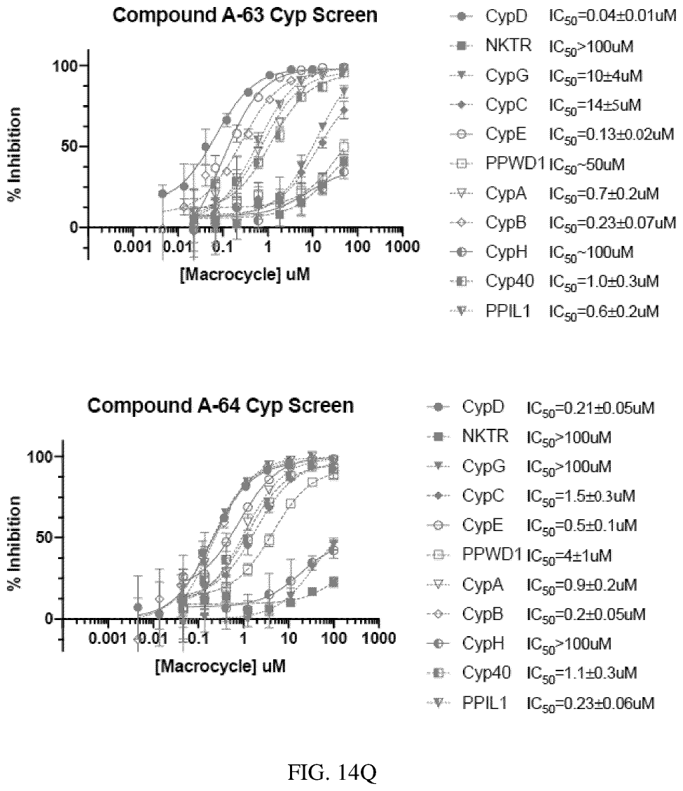

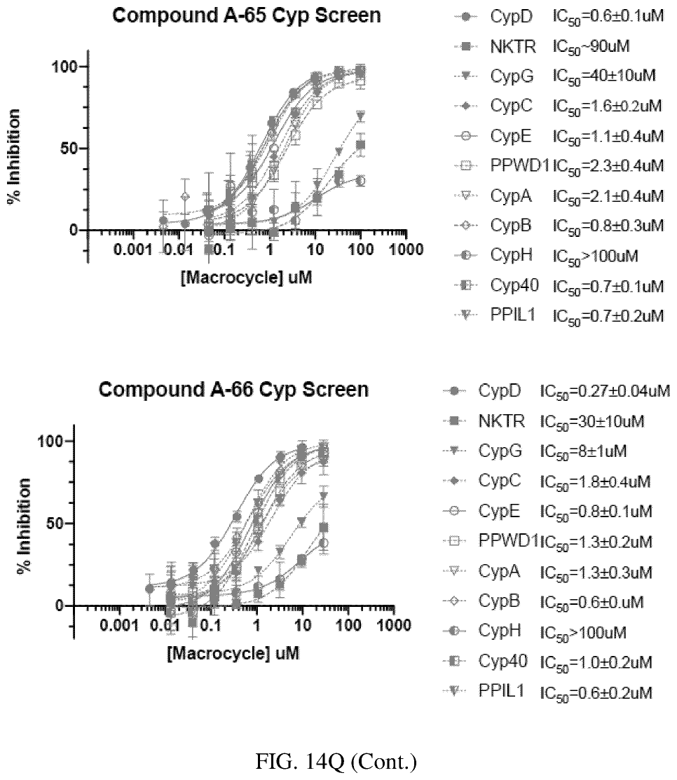

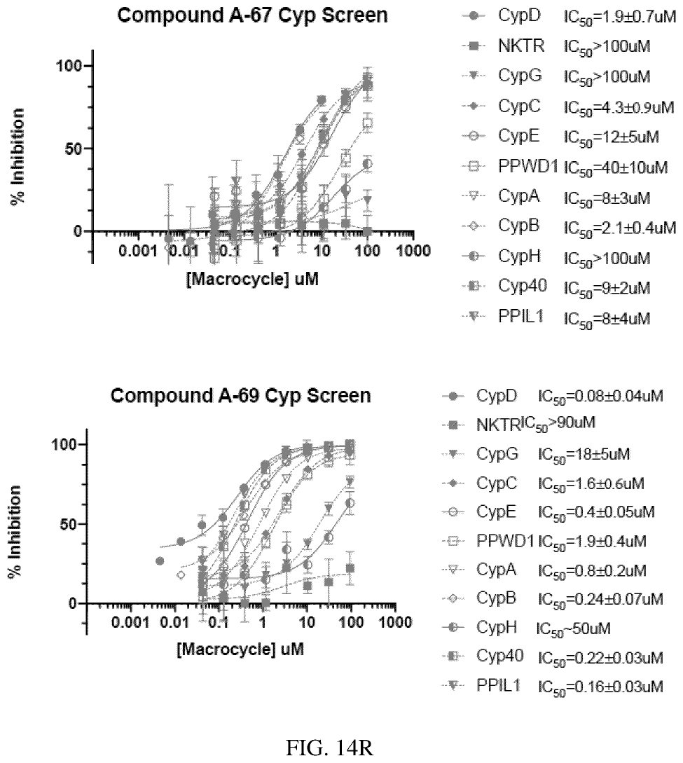

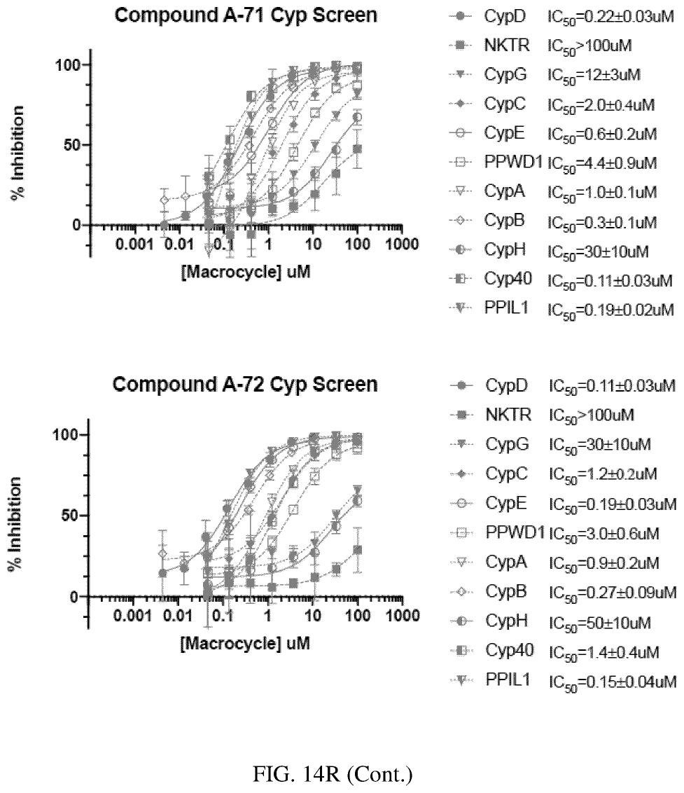

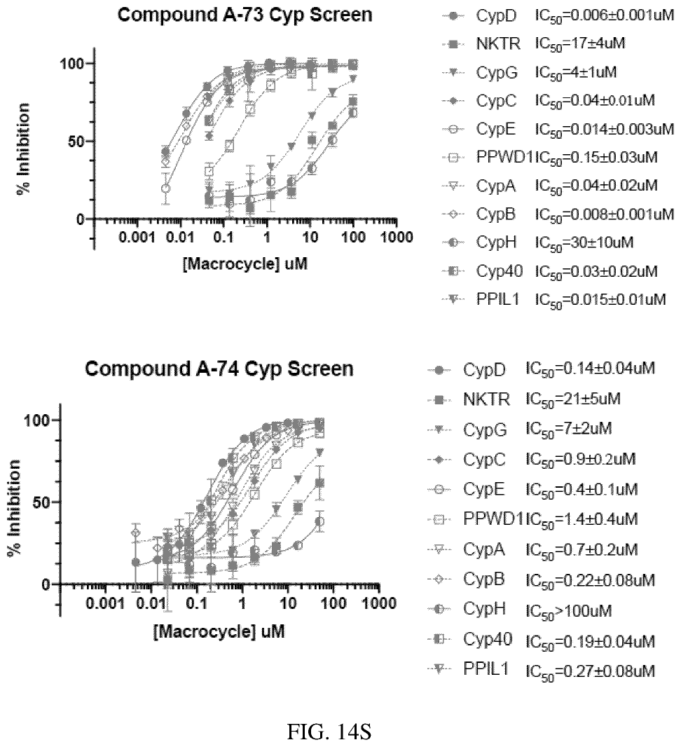

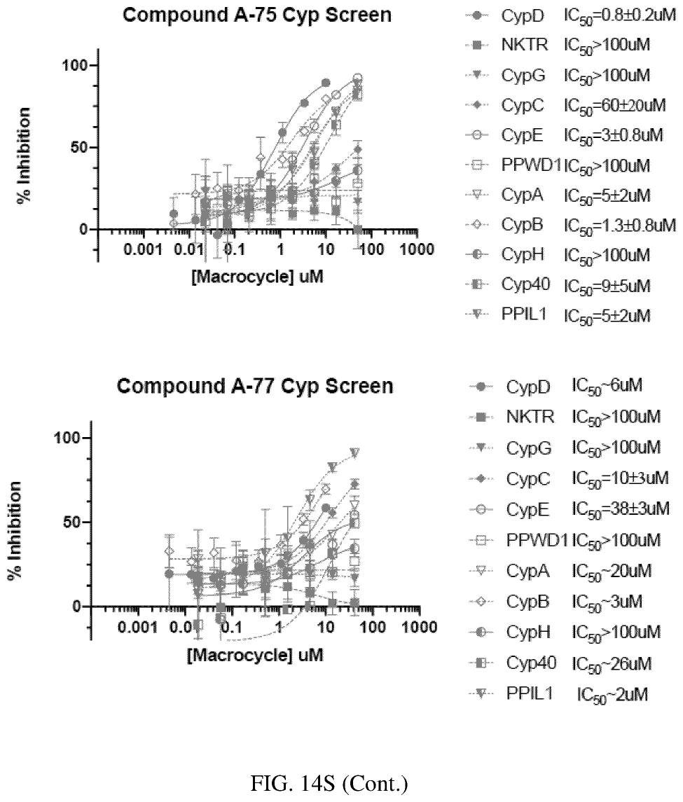

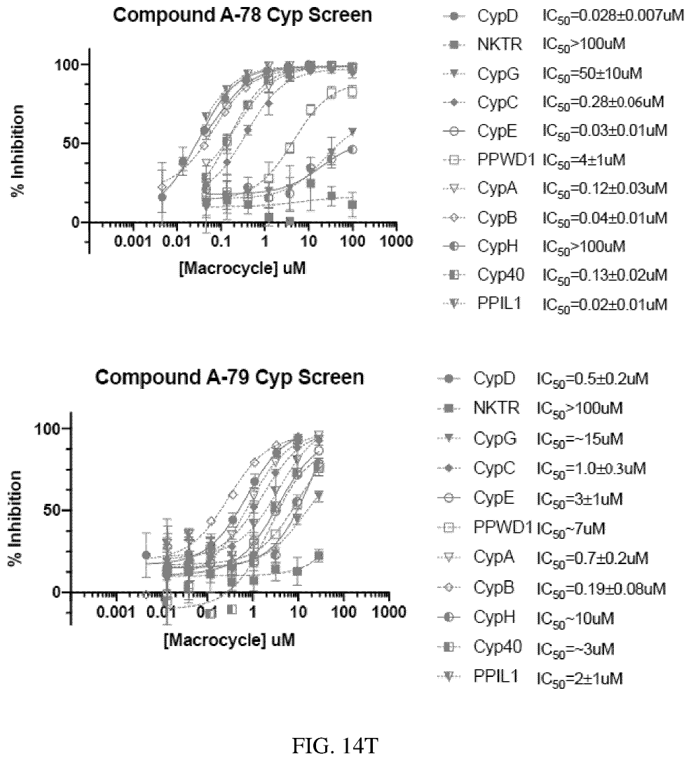

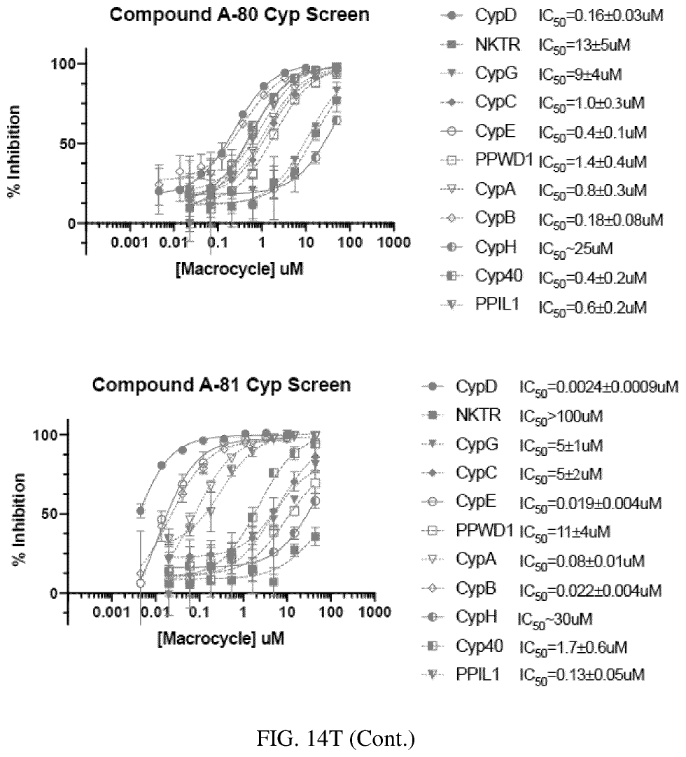

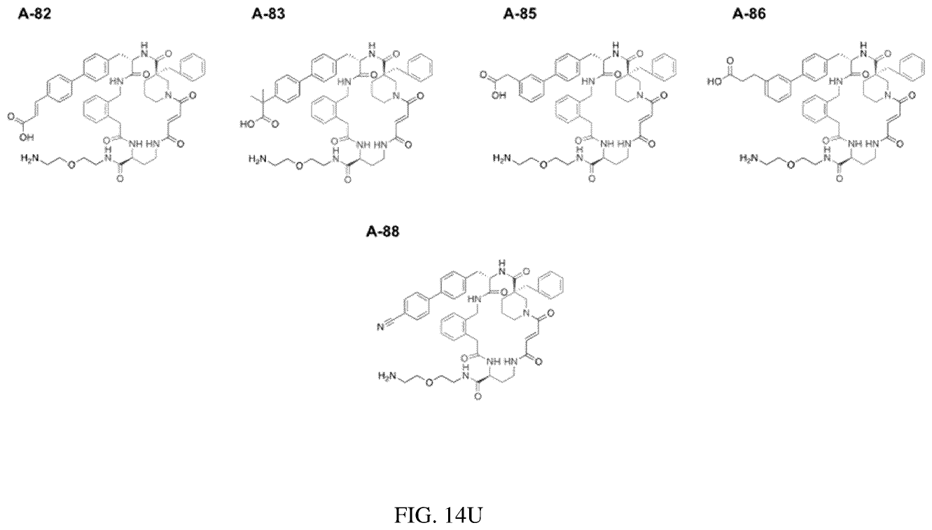

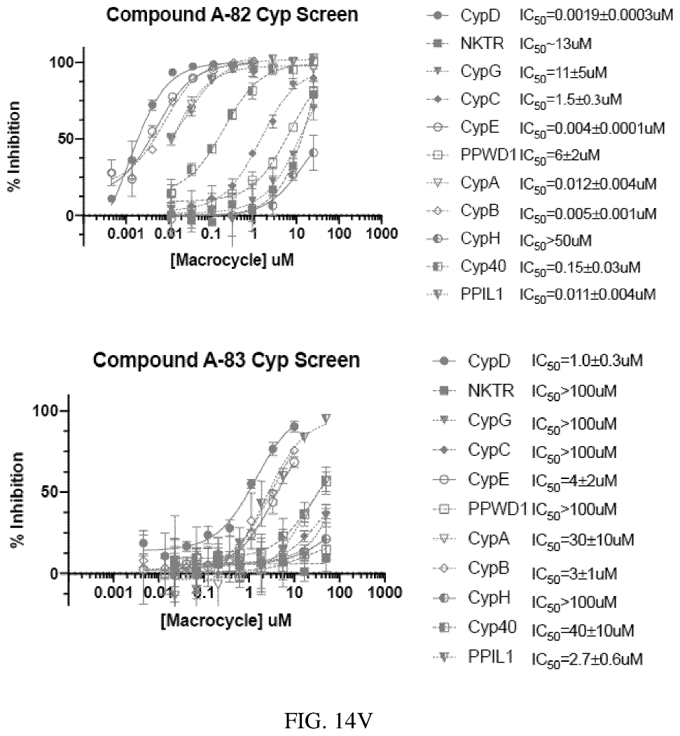

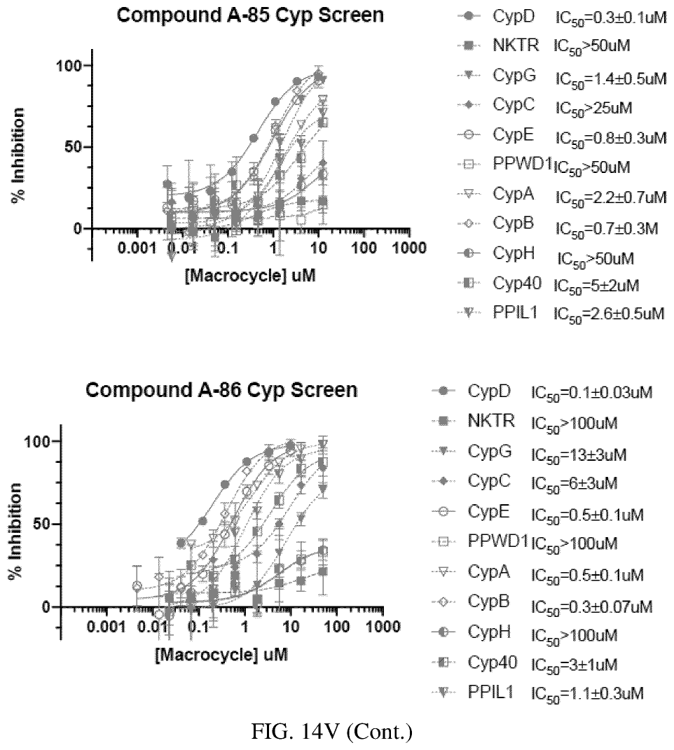

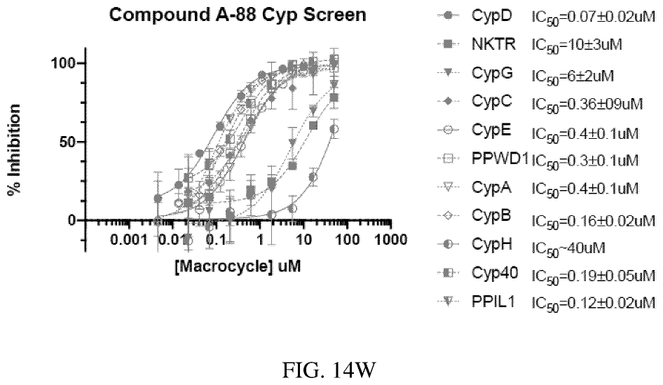

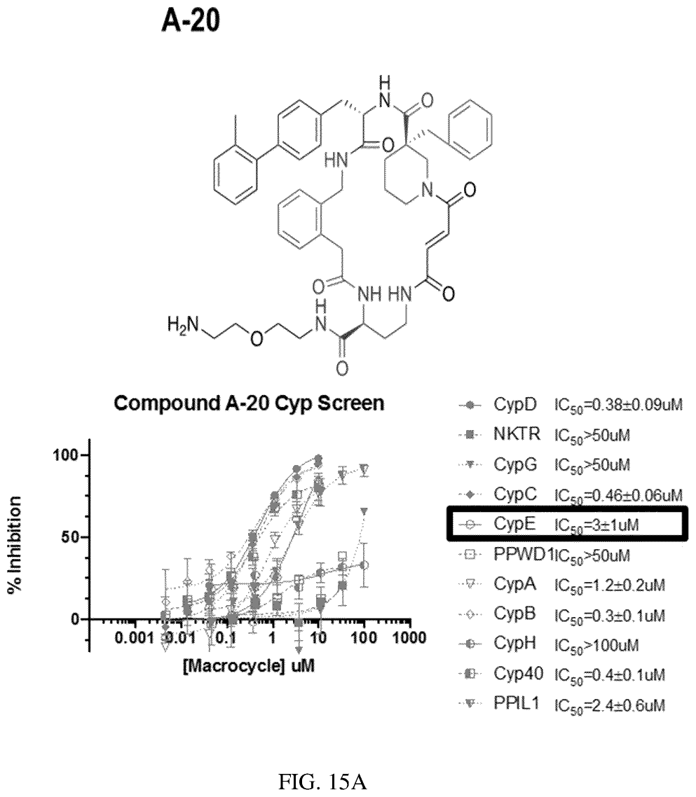

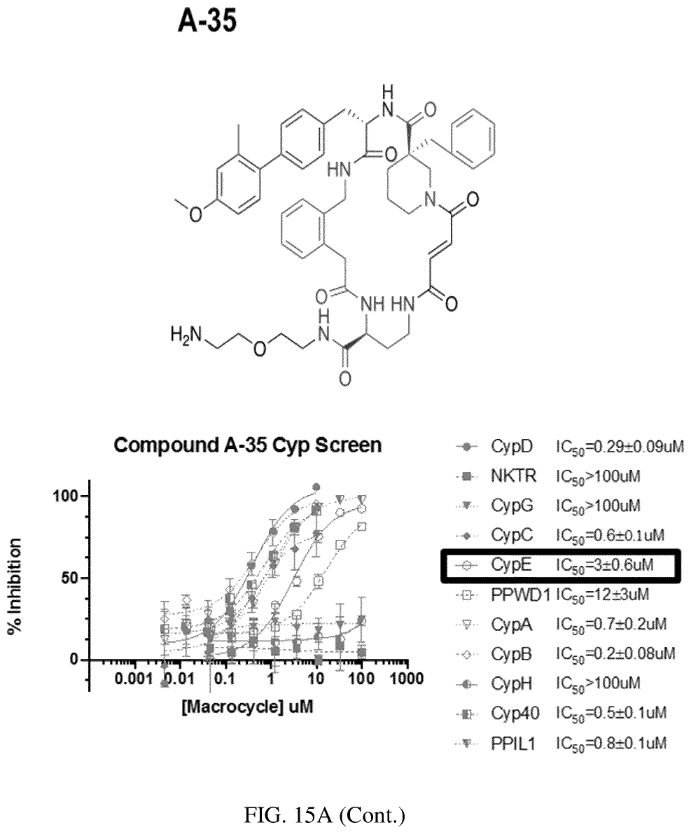

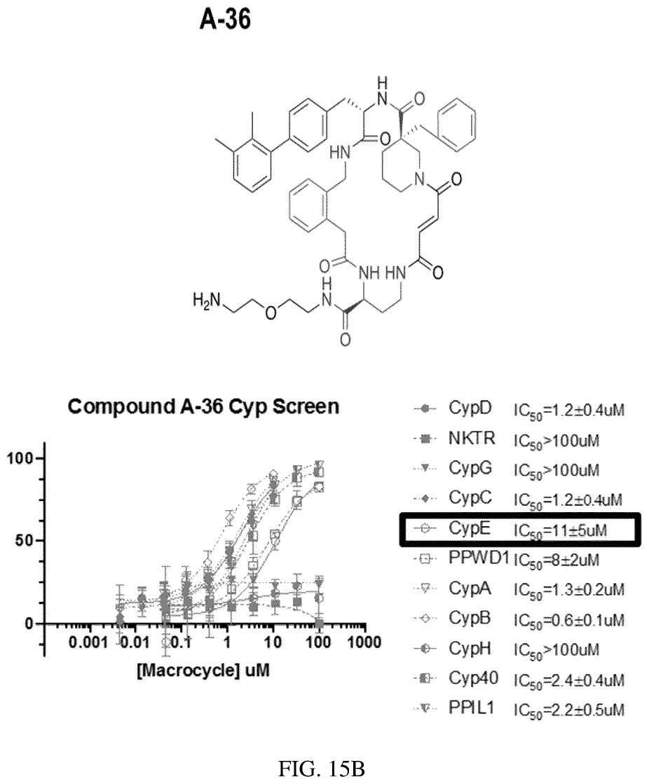

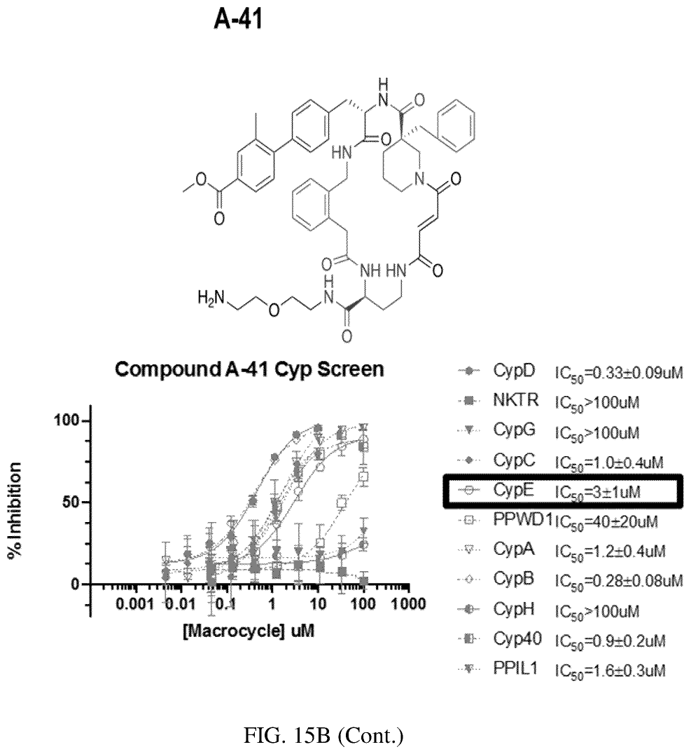

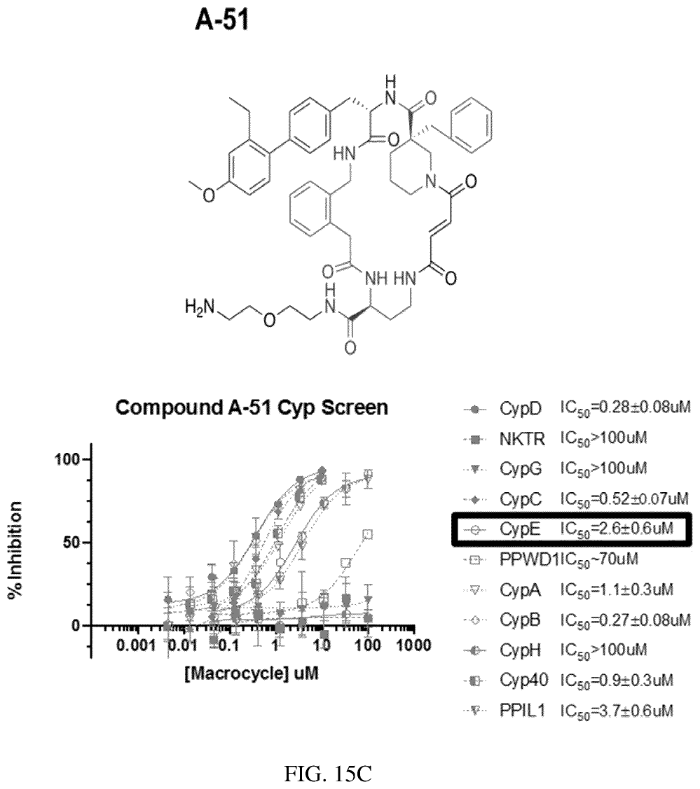

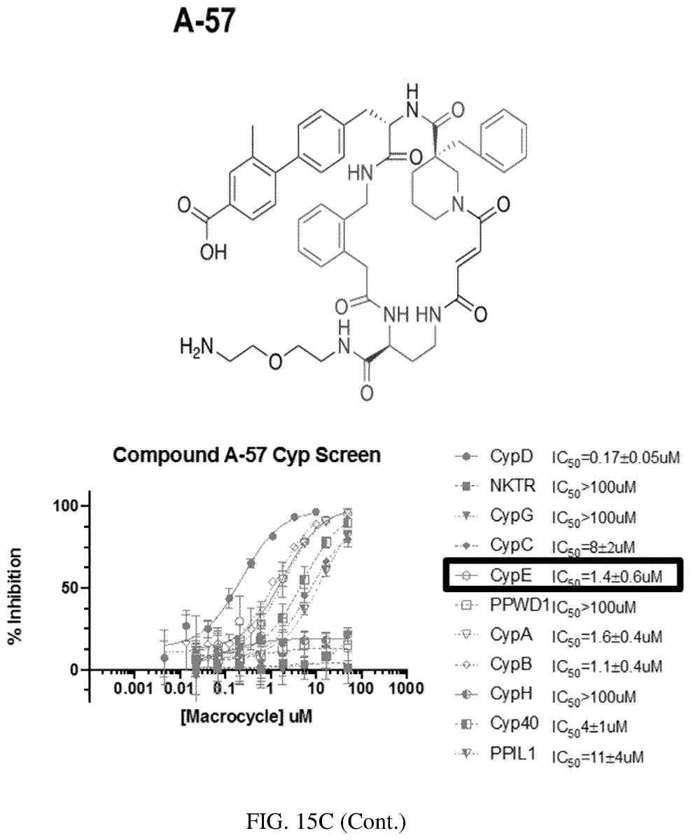

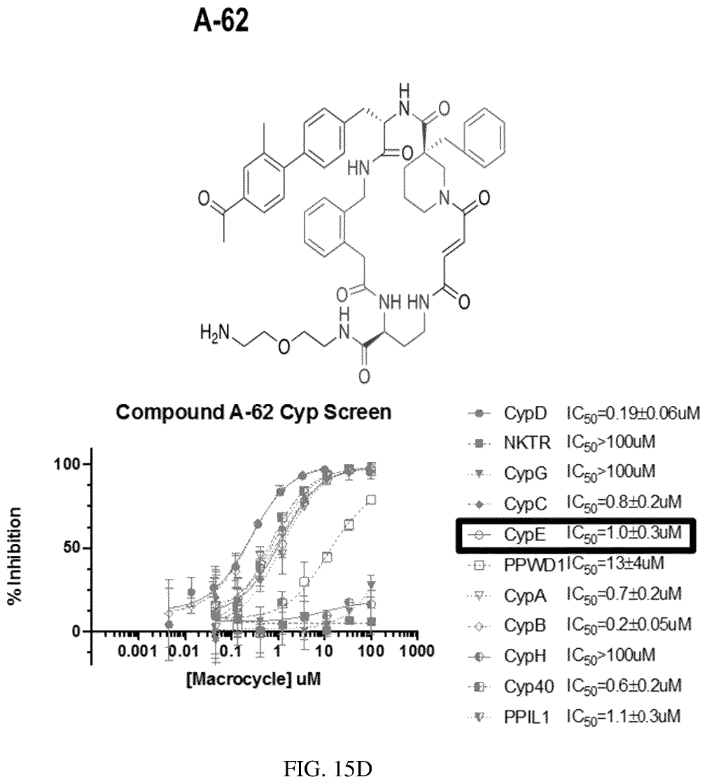

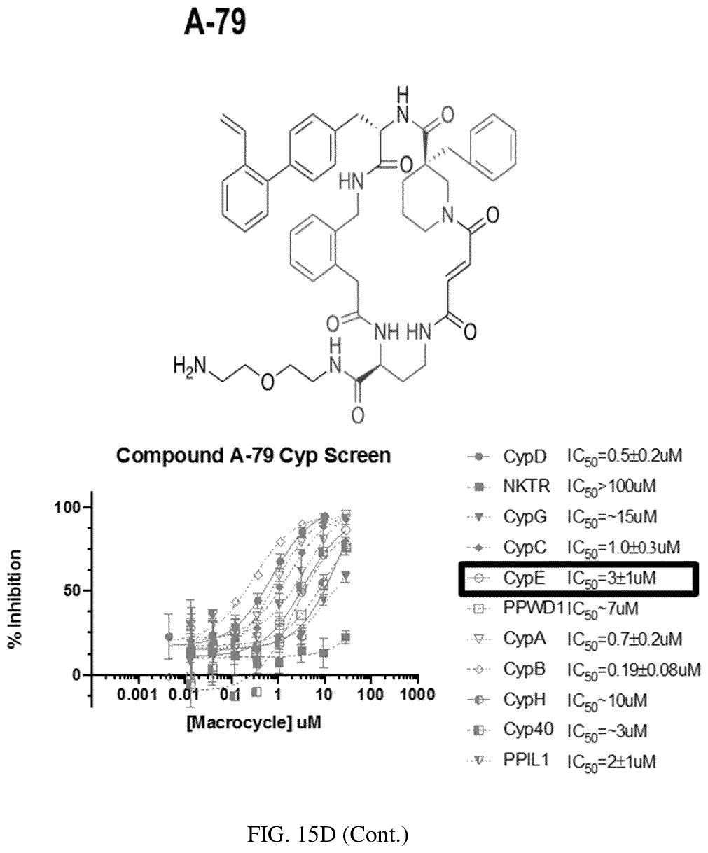

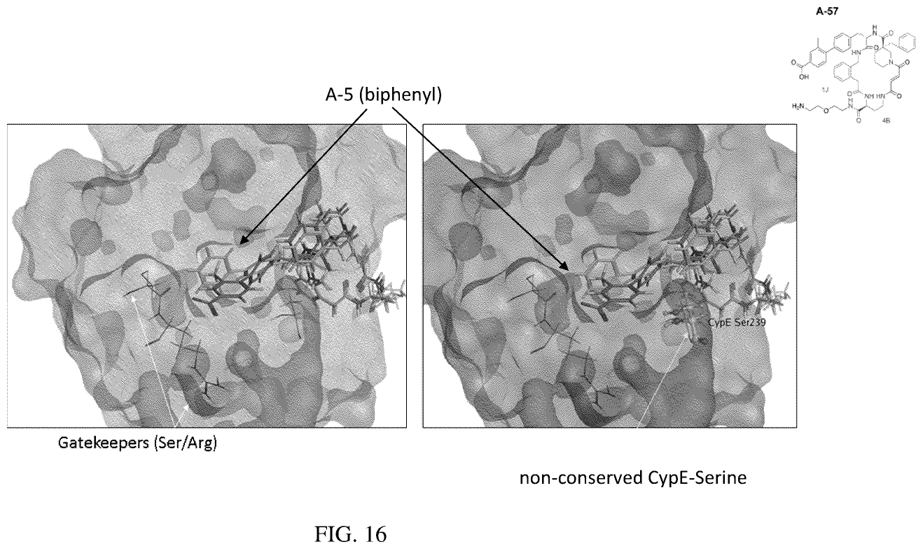

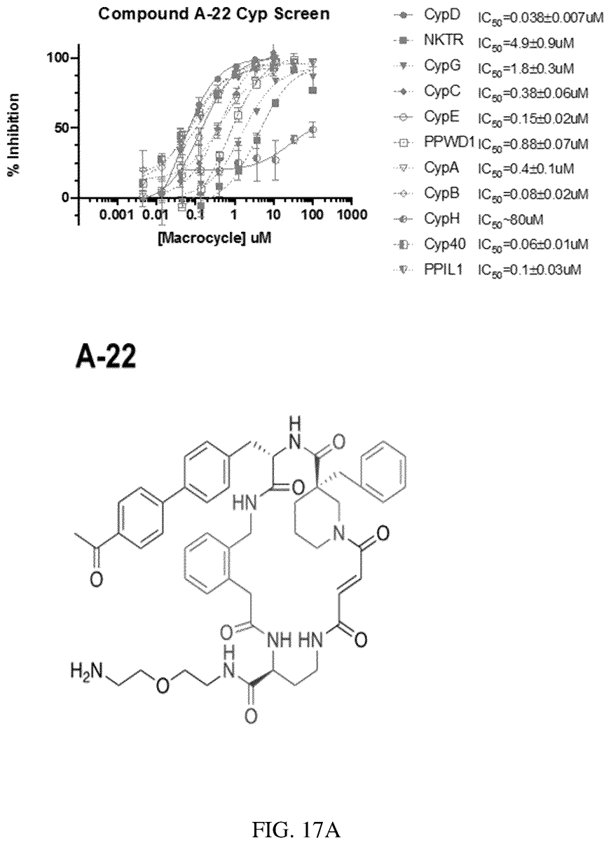

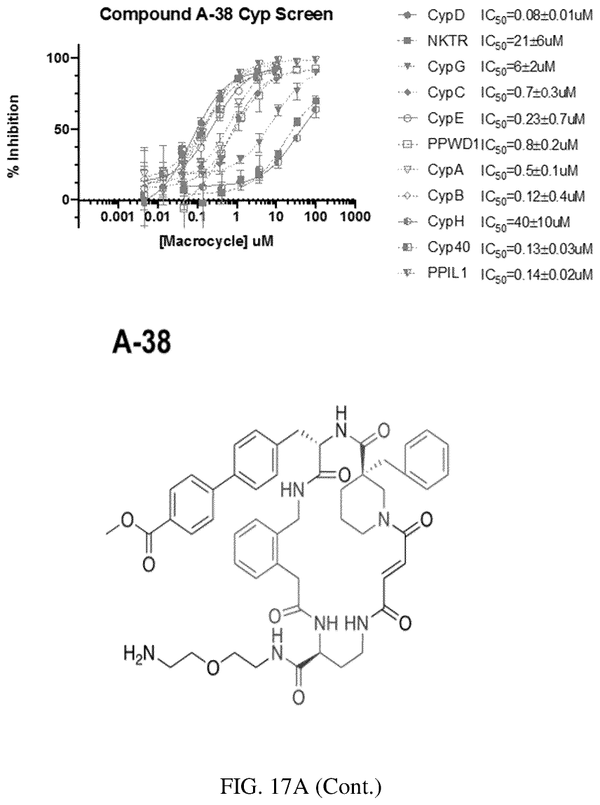

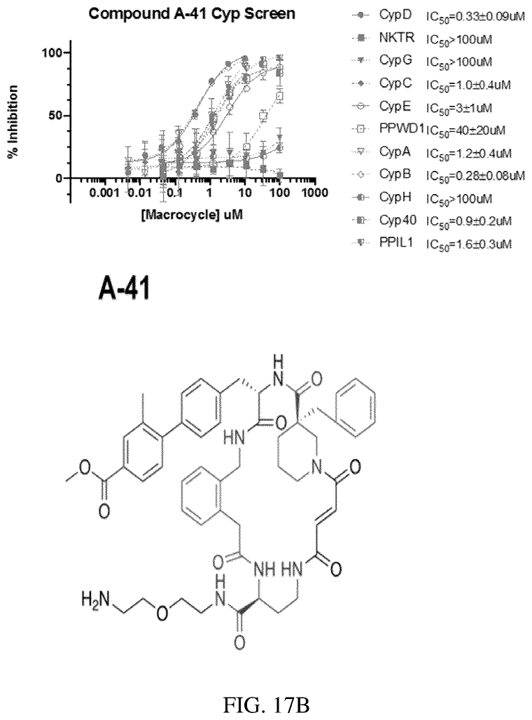

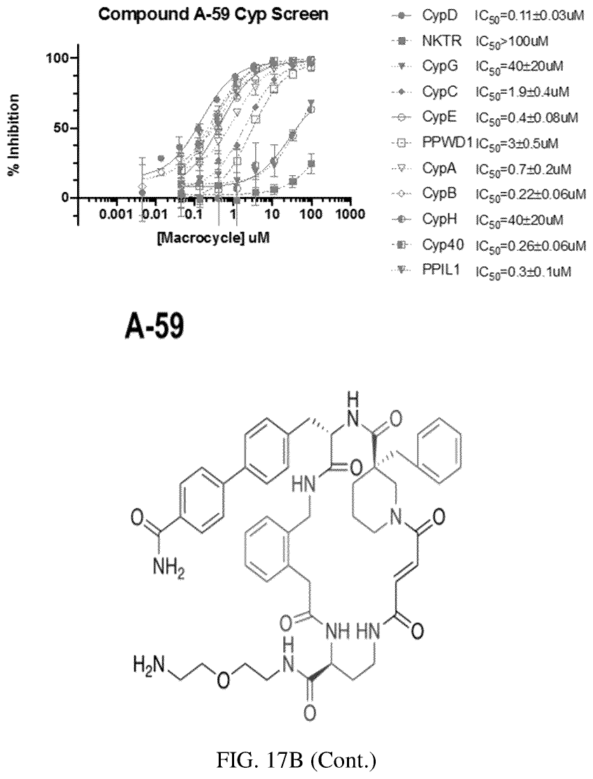

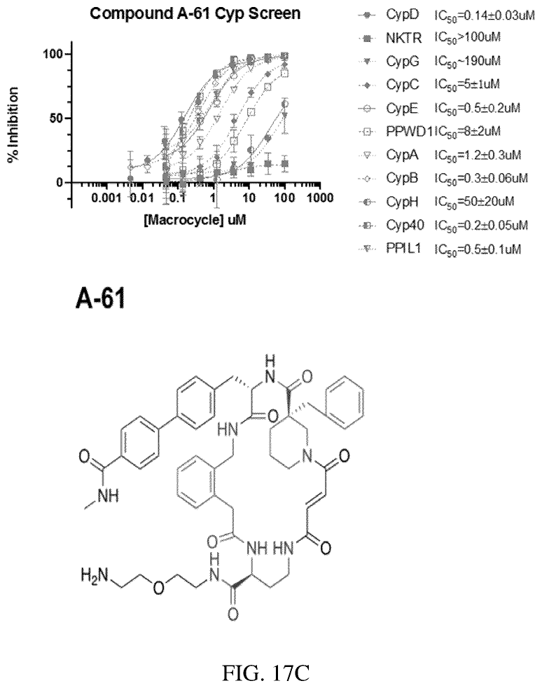

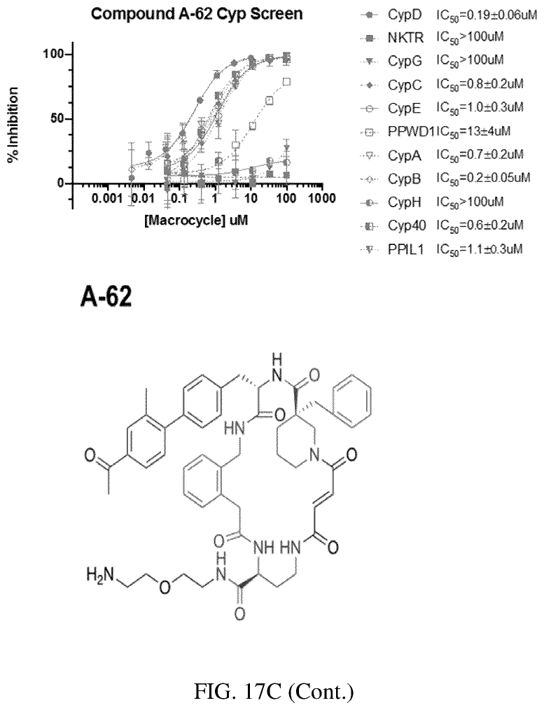

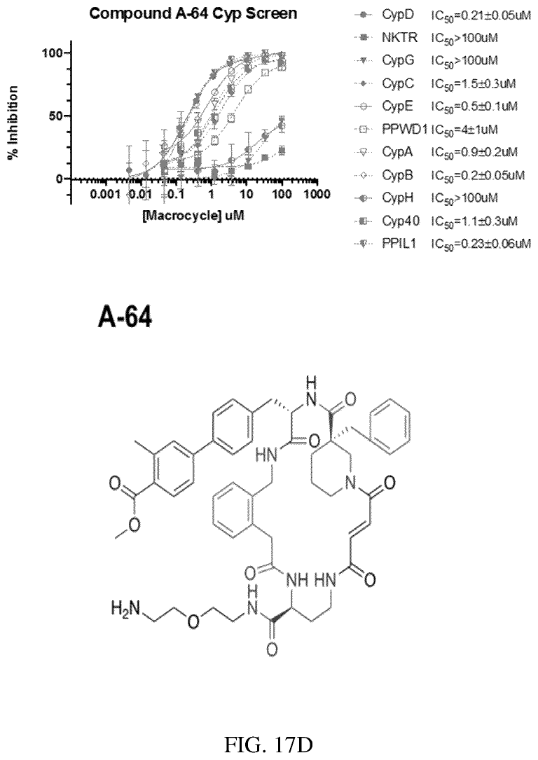

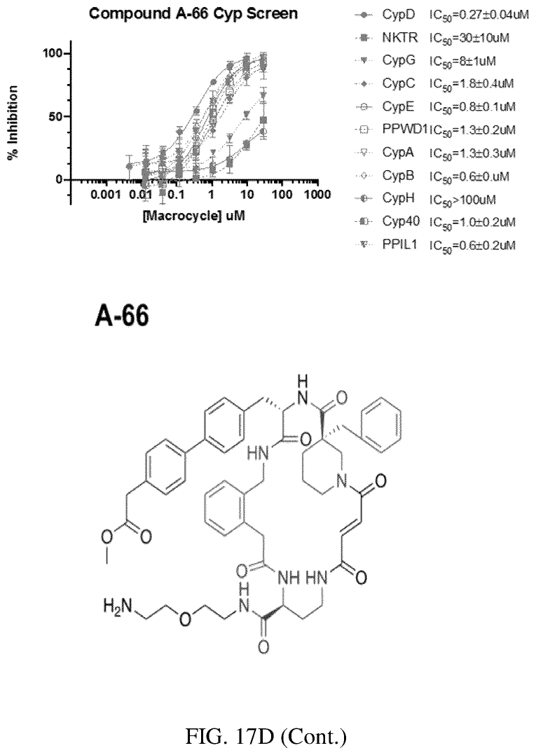

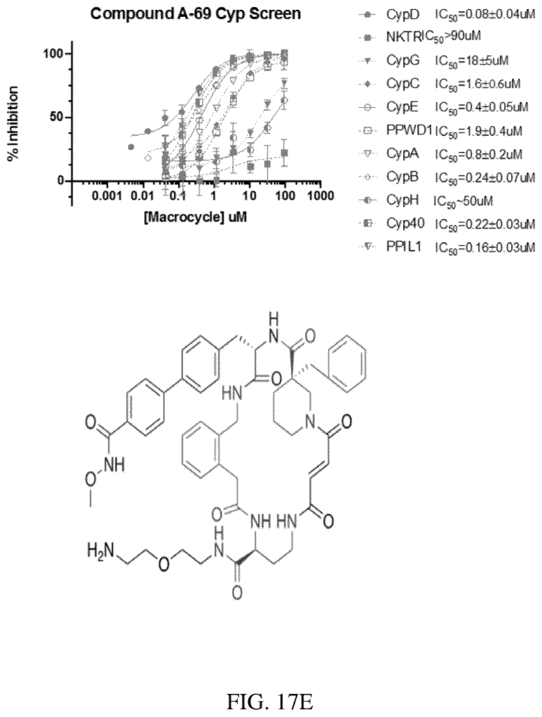

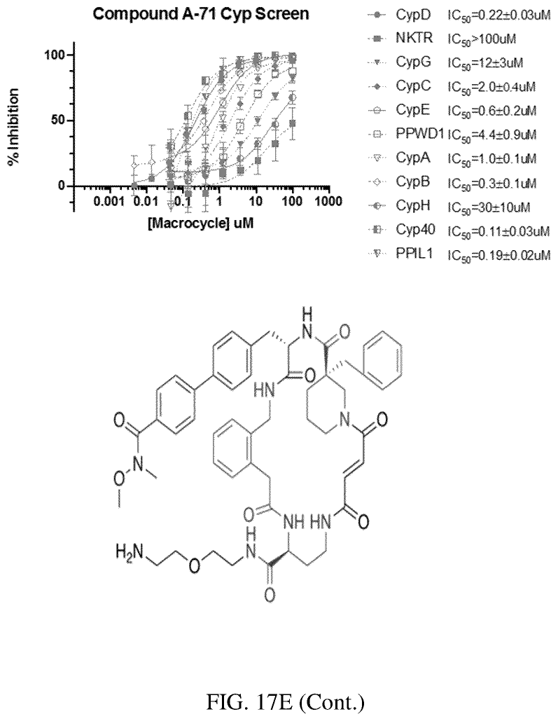

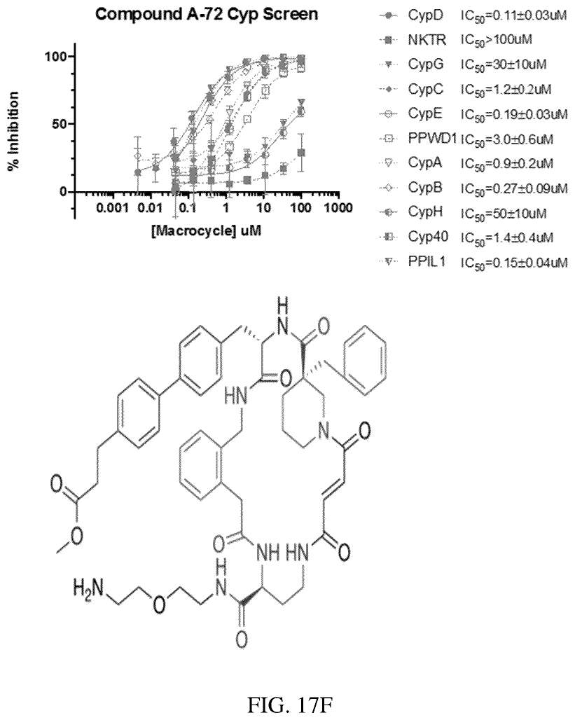

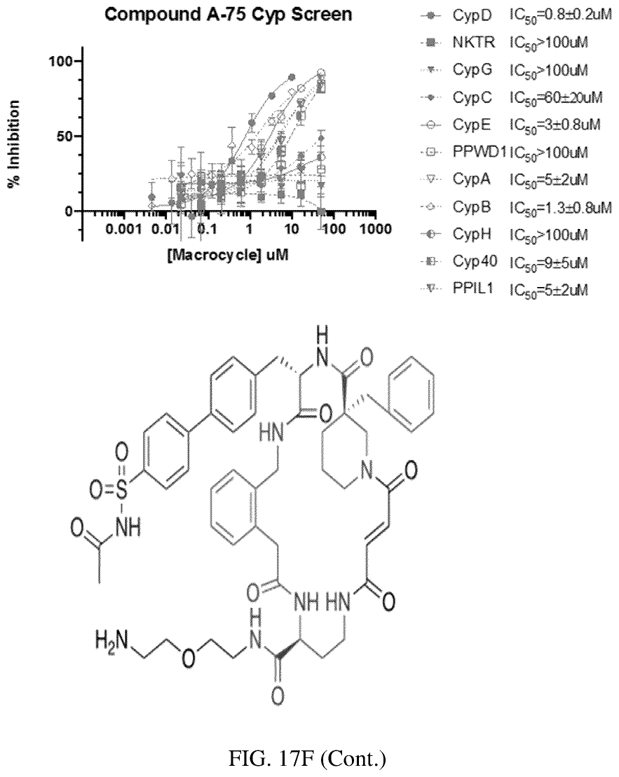

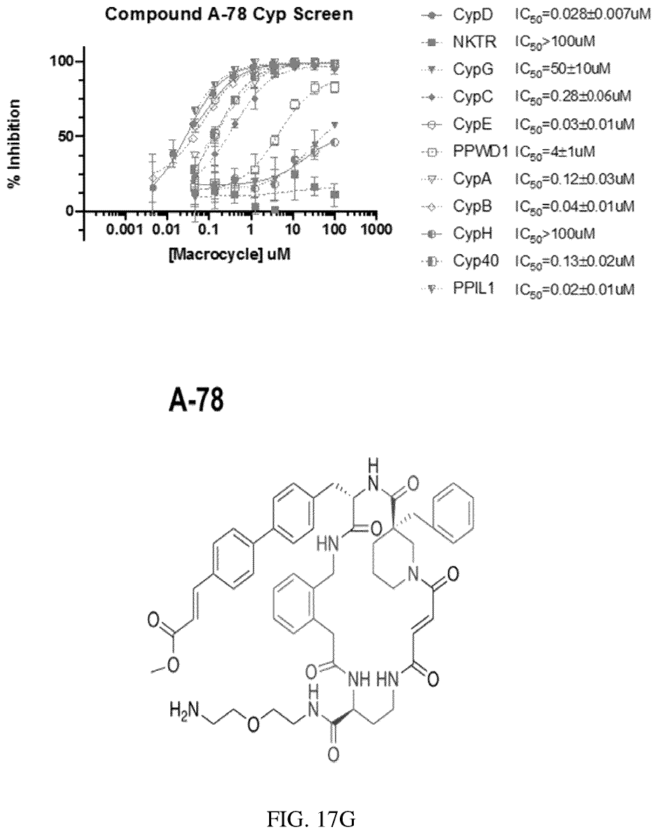

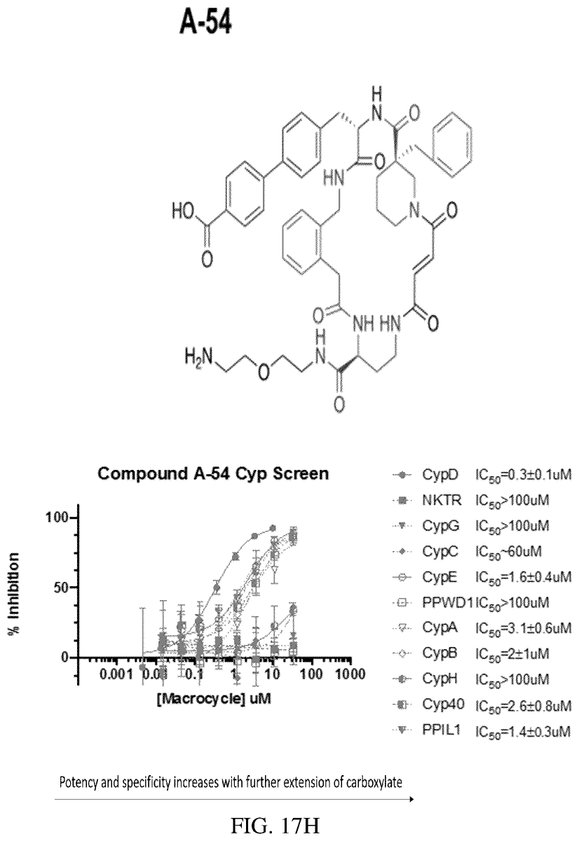

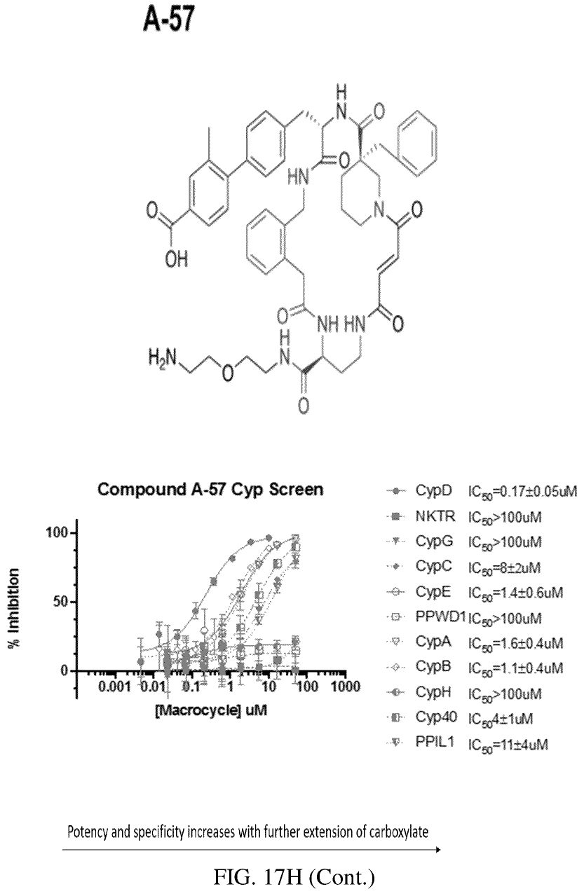

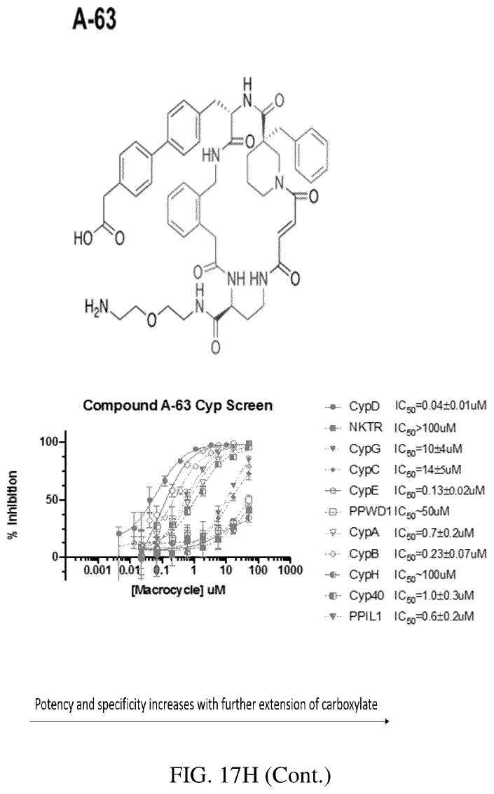

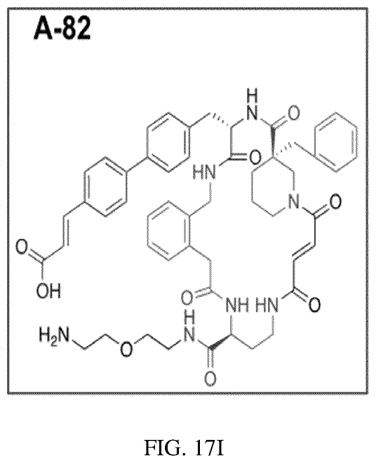

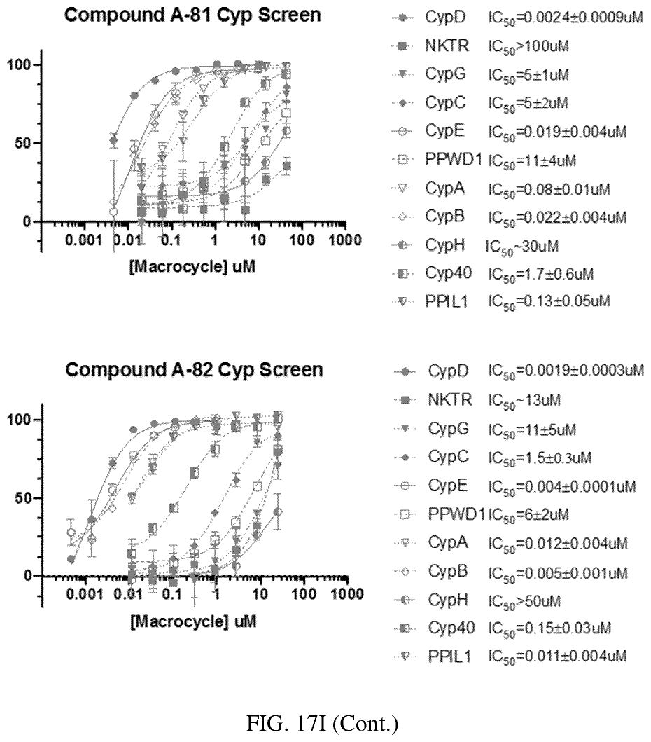

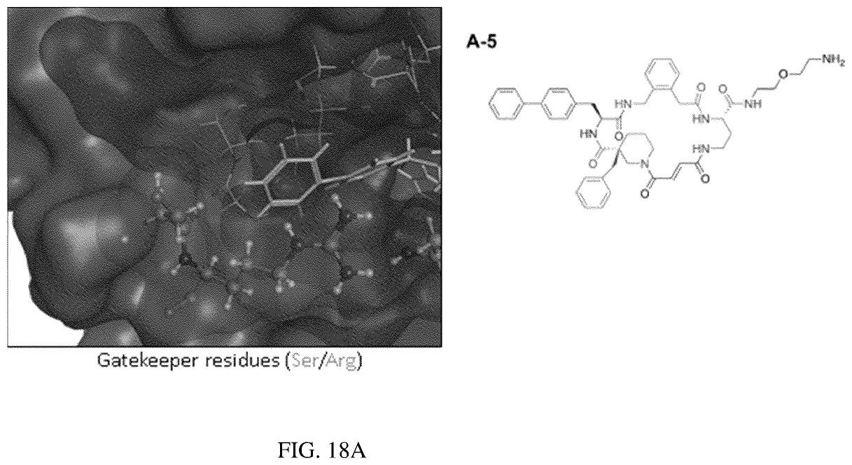

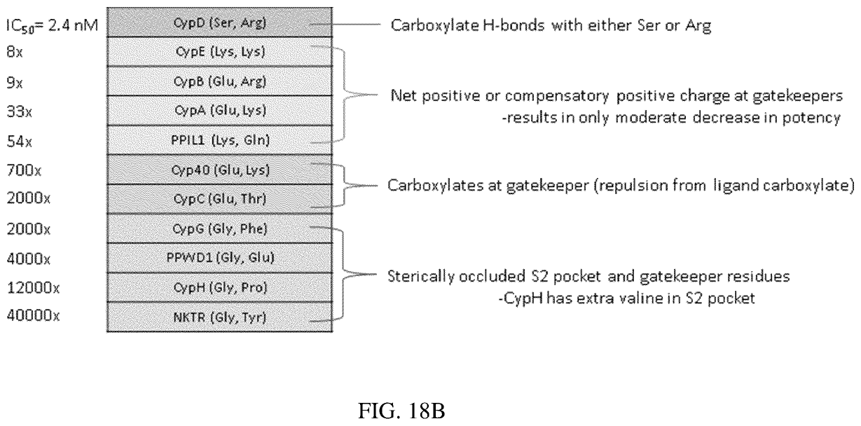

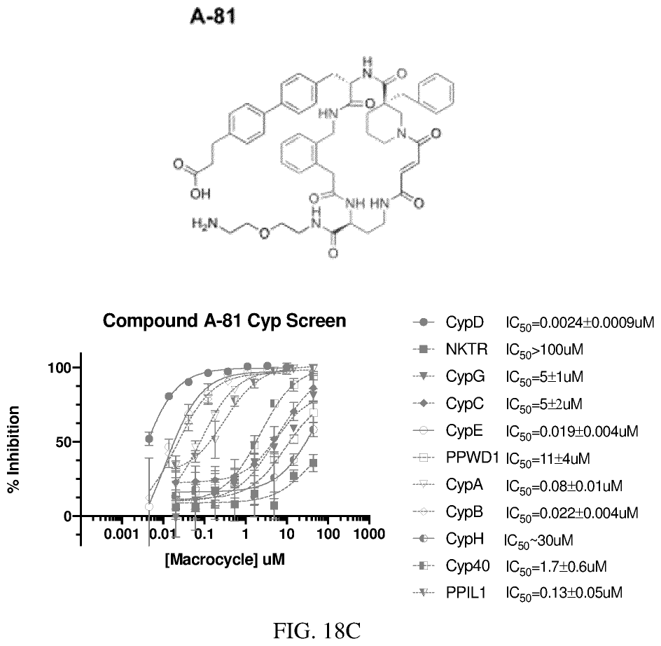

Selection on CypD and screening of library hits: To find novel CypD inhibitors, a selection was conducted with a DNA-templated library (DTS) of 256,000 macrocycles by incubating the library with immobilized His6-CypD on Ni-NTA functionalized beads. After washing away non-binders, the protein was eluted thus carrying any bound library members. This elution fraction was PCR amplified, barcoded, and submitted for high throughput sequencing to generate enrichment plots. Each data point in FIG. 4 represents enrichment of one macrocycle-barcode in the library compared to the original pre-selection library. Upon two replicate experiments, two families of macrocycles with good structure-activity relationships (SAR) were enriched, the HO**/HJ** and the JO** families.

Following the synthesis of the indicated library members, in vitro inhibition (IC₅₀) assays of these hits were conducted to obtain IC₅₀ inhibitory data. For the IC₅₀ assays, a chymotrypsin coupled PPIase assay using Suc-AAPF-AMC as the peptide substrate was used, whereby isomerization of a peptide substrate Suc-AAPF-AMC from the cis to trans conformation allowed for proteolysis via excess a-chymotrypsin, releasing the C-terminal coumarin fluorophore. Of the tested compounds, two macrocycles showed inhibition profiles, JOMBtrans and JOBBtrans, both of which have 10-40 uM IC₅₀ values and contain a conserved 3-carboxy-piperidine structure in the 3^{rd} building block. The weaker inhibition profile was further validated through Surface Plasmon Resonance experiments that garnered similar K_{d} values. Other non-inhibitor library members were also checked for binding via SPR, but produced the same profile as the prolyl isomerase assay.

TABLE 1
Exemplary compounds from DTS Library with inhibitory activity against
cyclophilins

| | |
|---|---|
| 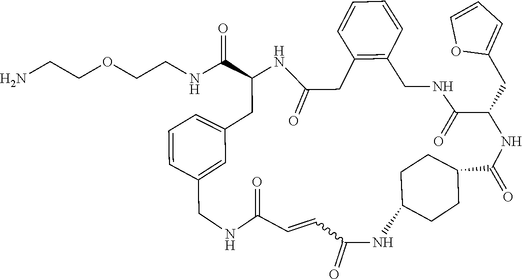 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1] | (JOGJ) |
| 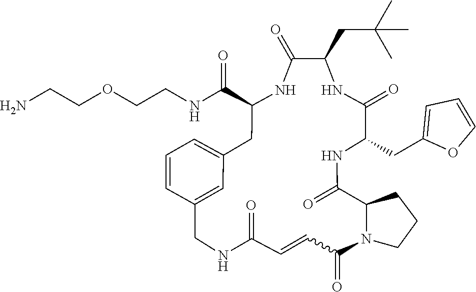 | (HOJJ) |
| 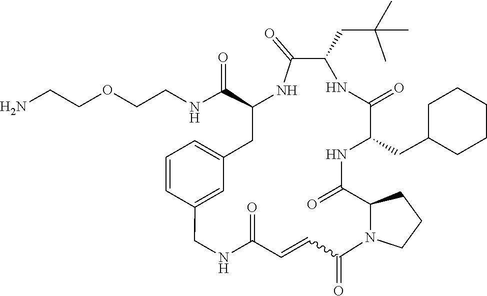 | (HJJJ) |
| 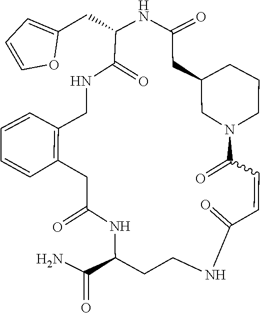 | (JOBB_A) |
| 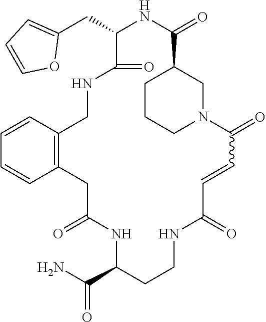 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1] | (JOMB_A) |
| 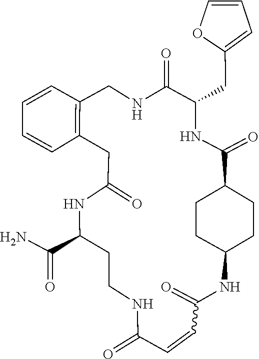 [SMILES: NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1] | (JOGB_A) |
| 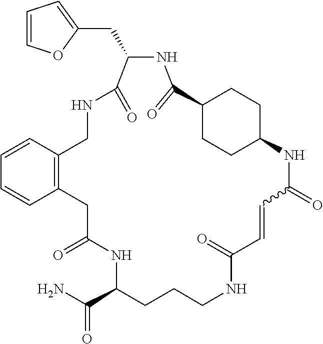 [SMILES: NC(=O)[C@@H]1CCCNC(=O)C=CC(=O)N[C@H]2CC[C@H](CC2)C(=O)N[C@@H](Cc2ccco2)C(=O)NCc2ccccc2CC(=O)N1] | (JOGC_A) |
| 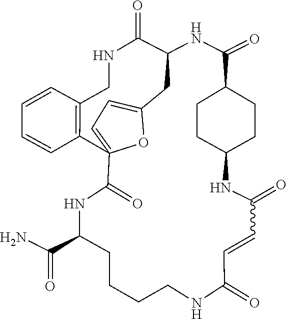 | (JOGD_A) |
| 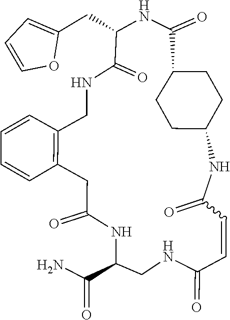 [SMILES: NC(=O)[C@@H]1CNC(=O)C=CC(=O)N[C@H]2CC[C@H](CC2)C(=O)N[C@@H](Cc2ccco2)C(=O)NCc2ccccc2CC(=O)N1] | (JOGA_A) |
| 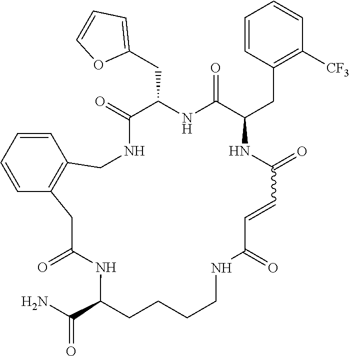 [SMILES: NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1] | (JOID_A) |
| 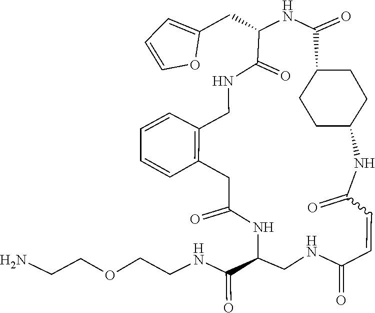 [SMILES: NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1] | (JOGA) |
| 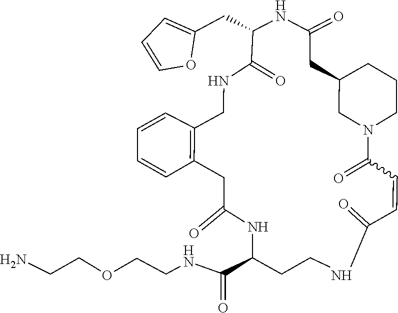 [SMILES: NCCOCCNC(=O)[C@@H]1CCNC(=O)C=CC(=O)N2CCC[C@H](CC(=O)N[C@@H](Cc3ccco3)C(=O)NCc3ccccc3CC(=O)N1)C2] | (JOBB) |
| 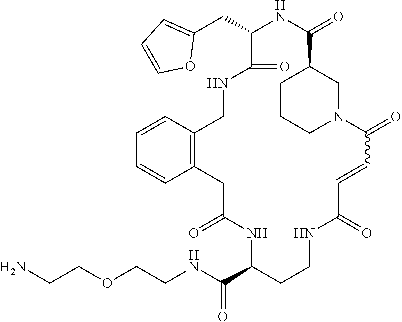 [SMILES: NCCOCCNC(=O)[C@@H]1CCNC(=O)C=CC(=O)N2CCC[C@H](C2)C(=O)N[C@@H](Cc2ccco2)C(=O)NCc2ccccc2CC(=O)N1] | (JOMB) |
| 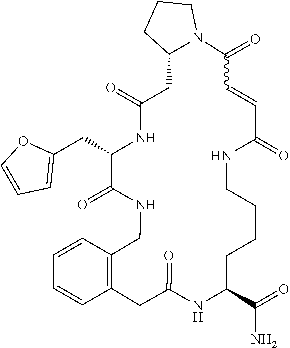 | (JOCD_A) |
| 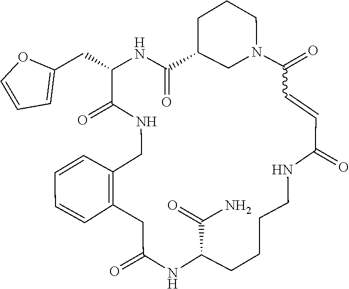 [SMILES: NC(=O)[C@@H]1CCCCNC(=O)C=CC(=O)N2CCC[C@H](C2)C(=O)N[C@@H](Cc2ccco2)C(=O)NCc2ccccc2CC(=O)N1] | (JOMD_A) |
| 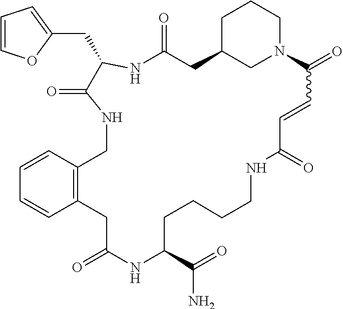 | (JOBD_A) |
| 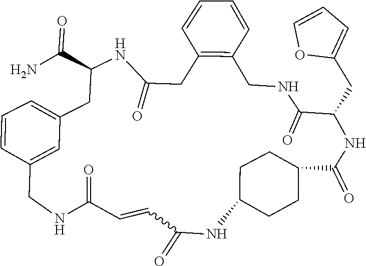 [SMILES: NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1] | (JOGJ_A) |
| 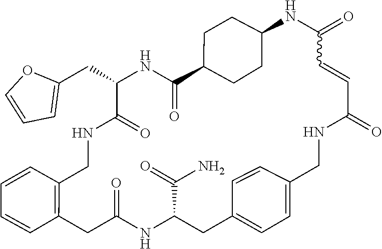 [SMILES: NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1] | (JOGK_A) |
| 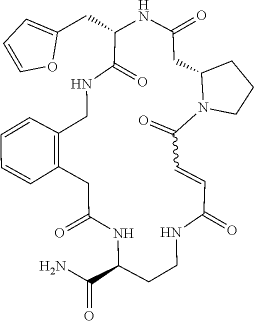 [SMILES: NC(=O)[C@@H]1CCNC(=O)C=CC(=O)N2CCC[C@H]2CC(=O)N[C@@H](Cc2ccco2)C(=O)NCc2ccccc2CC(=O)N1] | (JOCB_A) |
| 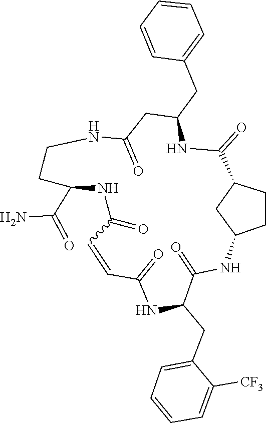 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1] | (CNIV_A) |
| 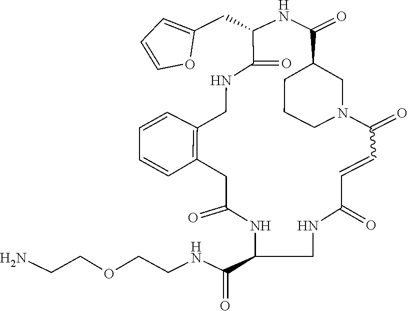 [SMILES: NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1] | (JOMA) |
| 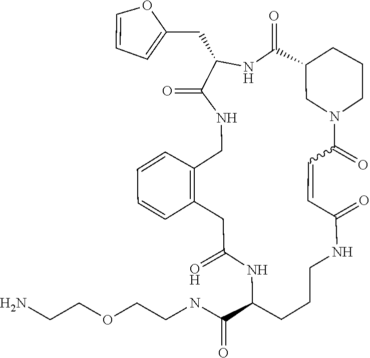 | (JOMC) |
| 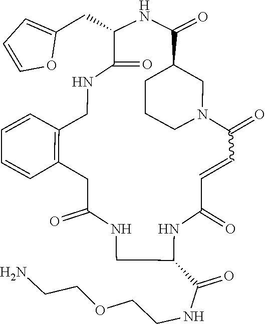 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1] | (JOME) |
| 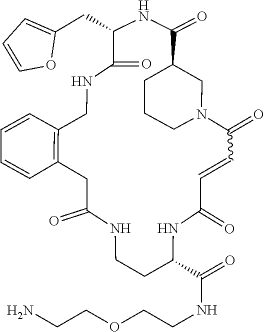 [SMILES: NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1] | (JOMF) |
| 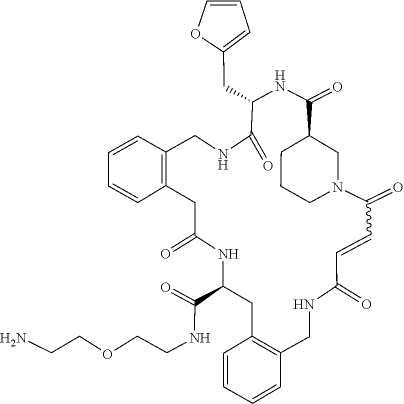 [SMILES: NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1] | (JOMI) |

Derivatization of JOMBtrans and analysis of binding modes from co-crystal structures: Using JOMBtrans as a basis for derivatization, a variety of analogs were synthesized in an attempt to garner further CypD potency. Several trends were elucidated including retention of the phenyl ring of the 1J building block, defined 22-23 atom macrocycle backbone, the trans conformation at the alkene, strict stereochemical requirements at each stereocenter, and modulatory of the furan at the 2^{nd} building block. One of the very good analogs involved substituting a benzyl group at the tertiary carbon on the piperidine ring, increasing potency to 200 nM (compound 49). Substitutions at this position with other functionalities also improved potency compared to JOMBtrans, but none to a greater degree than compound 49.

Subsequently, to determine if the compounds could both access the S2 pocket and display CypD specificity, JOMBtrans and compound 49 were screened against the 10 other cyclophilins with reported prolyl isomerase activity. Promiscuous inhibition was observed, exhibiting at most a ten-fold decrease in inhibition against other family members compared to CypD. Co-crystal structures of JOMBtrans and 49 were solved with CypD. JOMBtrans exhibits a dual-binding mode with CypD, where the phenyl ring on the 1J building block is buried in the active side and the furan sits at the precipice of the S2 pocket. Three further H-bonds with the macrocycle's backbone and CypD were observed along with a cation-n interaction. Compound 49 displayed a similar but optimized binding mode with CypD. The benzyl group on the piperidine ring altered the conformation of the macrocycle to open up two new hydrogen bonding interactions with CypD. This conformational shift can be attributed to the 75-fold increase in potency for 49 compared to JOMBtrans. Both compounds showed very promiscuous cyclophilin inhibition, which was attributed to the furan not accessing the S2 pocket and gatekeepers, most of its binding interactions occurring around the structurally conserved active pocket. A quick screen of an alternate JOMBtrans derivative (compound 27), which replaces the furan with a larger benzophenone, showcased an improvement in specificity for CypD. Almost complete ablation of inhibition profiles for NKTR, CypG, PPWD1, CypH, was observed along with decreased potency for other cyclophilins. This improvement in specificity compared to JOMBtrans signified that more CypD specific compounds can be garnered by derivatization into the S2 pocket.

Initial A-Series derivatives and co-crystal structures: Using conclusions from solved co-crystal structures and specificity profiles, a new series of macrocycles (A-series) was created that utilized the increased potency from the benzyl-piperidine of 49 coupled with moieties that replaced its furan group. The initial series of A-series macrocycles displayed a variety of specificity profiles. Macrocycles with large functionalities (benzophenone, biphenyl, tertbutyl-Phe for compounds A-1, A-5, and A-9 respectively) abolished inhibition for NKTR, CypG, PPWD1, CypH, mimicking the profile of compound 27, albeit with much better potency. To confirm that these larger substituents reached deeper in the S2 pocket and could access the gatekeeper residues, co-crystal structures of compounds A-1 and A-5 were solved with CypD, along with compound A-6 which has a comparatively smaller substitution. Co crystal structures of these three compounds showed the same binding mode as compound 49. However, as larger moieties were introduced, both deeper binding into the S2 pocket as well as a greater degree of ‘flipping’ the gatekeeper residues out of the pocket was observed. For example, A-1's benzophenone completely removed the gatekeeper residues from the pocket while the 3-methyl-phenylalanine of compound A-6 allowed retention of the gatekeeper residues in the pocket. A-5 represented a middle-ground, whereby it still reached deep into the S2 pocket while also retaining close proximity to the gatekeeper residues.

A-Series derivatization to gain access to CypD specific inhibitors: From the co-crystal structure of compound A-5 with CypD, biphenyl derivatives containing moieties designed to interact with CypD gatekeeper residues, Ser81 and Arg82, were synthesized. Hydrogen-bond acceptor functional groups were incorporated at the para position of the biphenyl group, as the crystal structure suggested that would be the most efficient way to access interactions with gatekeeper residues. A variety of functional groups were incorporated, including carbonyl, alkyl, alkoxy, and alcohol substituents along with various biphenyl heterocycles. Just like compound A-5, these derivatives all showed significant reduction in potency for NKTR, CypG, PPWD1, and CypH. CypC, CypE, CypA, CypB, Cyp40, and PPIL1 all had varying degrees of selectivity profiles depending on the substituent on the biphenyl group.

The A-series of macrocycles exhibited two trends from diversification. First, compounds with ortho-alkyl-biphenyl moieties showed a consistent 10-fold decrease in potency for CypE when compared to CypD. Second, p compounds with ara-substitutions with carbonyl like moieties, including ketones, amides, sulfones, esters, hydroxamic esters, and carboxylates exhibited varying drops in potency for CypC, CypA, CypB, Cyp40, and PPIL1. In particular, compounds with carboxylate substituted biphenyl groups showcased the best specificity profiles, as shown in compounds A-54 and A-57. Extension of the carboxylate via a methyl, ethyl or vinyl linker drastically increased the potency of these compounds for CypD and eliminated significant inhibition profiles for the other tested cyclophilins. A very good compound, A-81, contained a para-ethylcarboxy functionality that provides 2.5 nM potency and a drastic preference for inhibition of CypD.

Binding modes for inhibitors with improved specificity profiles: A Co-crystal structure of A-57 with CypD showed both ortho-alkyl biphenyl and para-carboxy biphenyl moieties, which explained the increased selectivity profile. Ortho-alkyl substituents slightly altered the binding mode of the biphenyl group, whereby the methyl group of A-57 buried itself in the base of the S2 pocket, thereby pushing the biphenyl group further out of the pocket. It was consistently observed that this alkyl substitution afforded around a 10-fold decrease in potency for CypE. Pushing the biphenyl group closer with an ortho-alkyl group created a repulsive interaction in CypE that would normally be a hydrophobic interaction in CypD.

Although ortho-alkyl biphenyl groups occupy a slightly different binding mode than regular biphenyl groups, the co-crystal structure of A-57 showed that para-carboxy biphenyl groups are placed near the gatekeeper residues of CypD. In this particular instance, the carboxylate fully flipped the Arg gatekeeper out of the S2 pocket while positioned near the Ser gatekeeper. Due to having a negatively charged carboxylate near the gatekeeper residues, a marked decrease in potency was observed for many cyclophilins compared to CypD, explained below.

Drawing conclusions from cyclophilin gatekeeper residues and inhibitor functional groups: As exhibited by compounds A-54, A-57, A-63, A-81, and A-82, para-carboxy biphenyl moieties placed within the S2 pocket provided cyclophilin selectivity profiles showing preference for CypD. Upon analysis and modeling into CypD, it was hypothesized that the family of tested cyclophilins fell into three broad categories based on their potency against the compound A-81. The first group of cyclophilins included NKTR, CypG, PPWD1, and CypH which contain relatively occluded S2 pockets from the gatekeeper residues or a combination of the gatekeepers with nearby residues. Nearly total ablation of inhibition was achieved for these cyclophilins by building sterically bulky moieties into the compounds. The second group included Cyp40 and CypC, which have sterically accessible S2 pockets just like CypD but have acidic moieties that repulse the carboxylates built into the compounds. The third group encompassed CypB, CypE, CypA, and PPIL1 which have varying degrees of selectivity compared to CypD. The variety of potencies was owed to gatekeeper residues that are similar to CypD (such as CypB) or that contain a varying number of positively charged regions that can interact with the carboxylate of the inhibitors.

Design inhibitors specific to other cyclophilins: Derivatization of compound 49 into the ‘A-series’ macrocycles provided a scaffold by which functional groups that bind in the S2 pocket and interact with gatekeeper residues were diversified. These new inhibitors can target disease models by which the mPTP has been implicated through oxidative damage. Given the ubiquity of oxidative damage in the mechanism of cell necrosis, offering an inhibitor that can target this mechanism via the mPTP-CypD interaction serves as a treatment method for multiple diseases. These inhibitors can be applied in animal models of neurodegeneration, as oxidative stress can cause irreversible damage to cells in the nervous system.

### Example 3. Additional Biological Assays of Compounds

### Further Exemplary A-Series Macrocycle Compounds

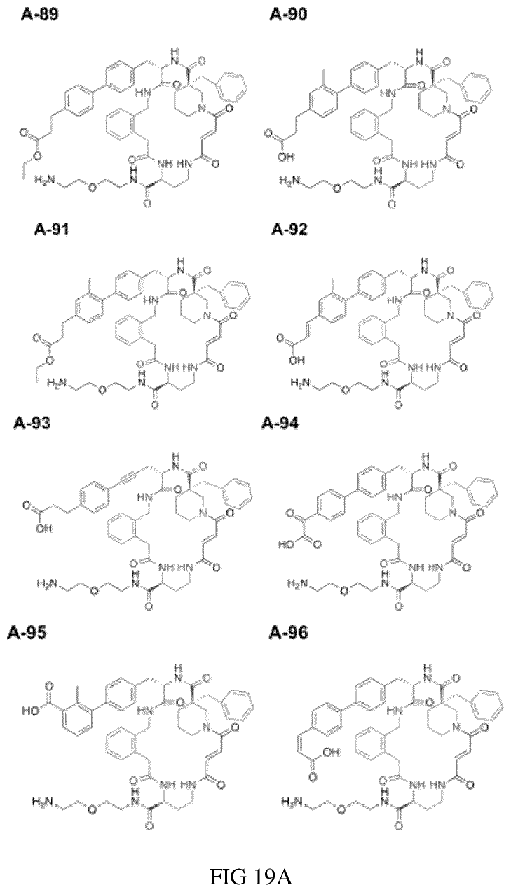

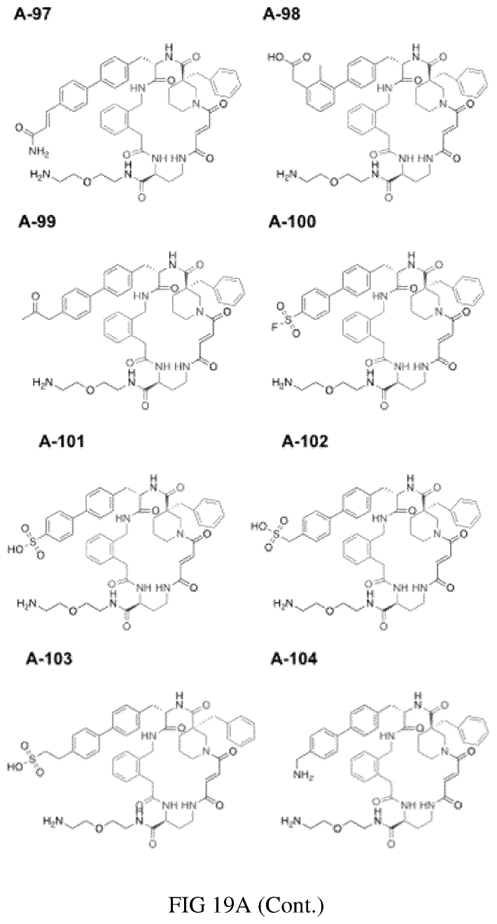

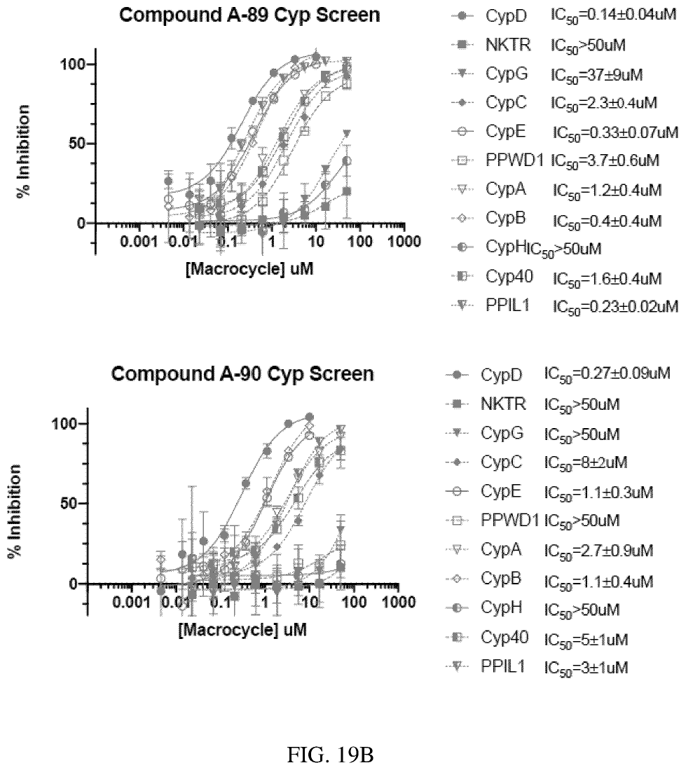

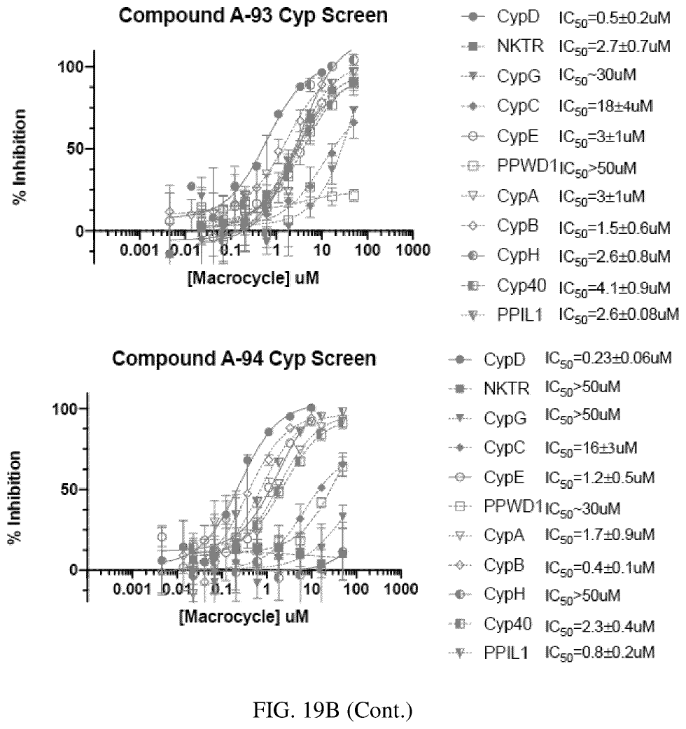

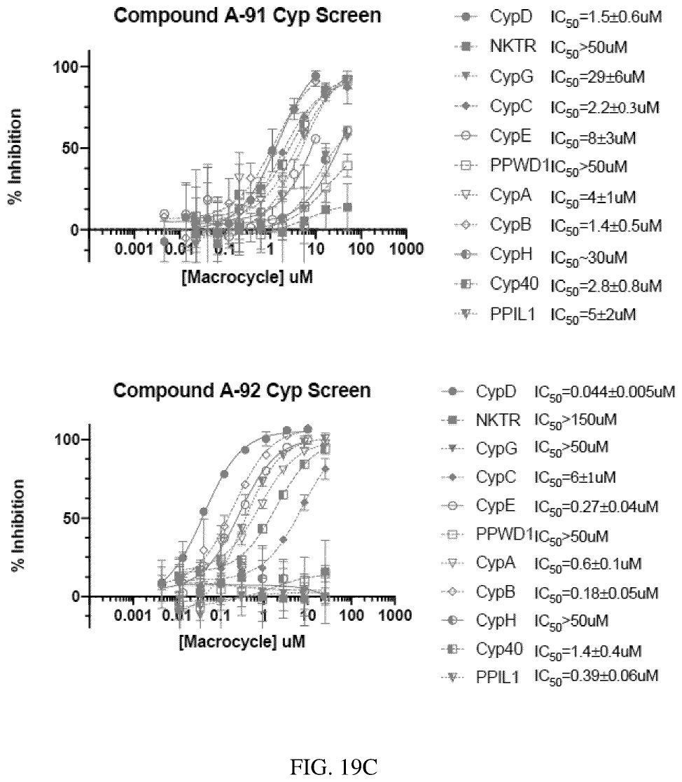

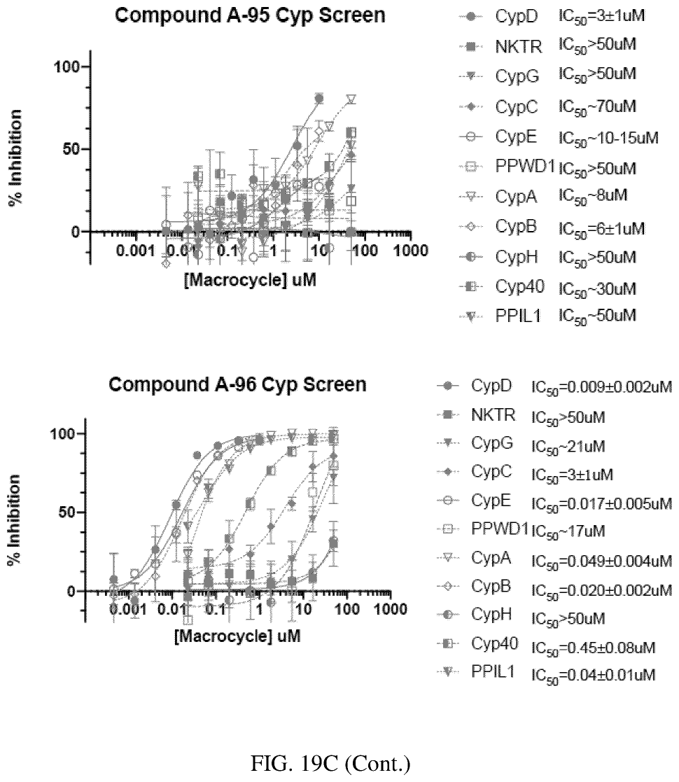

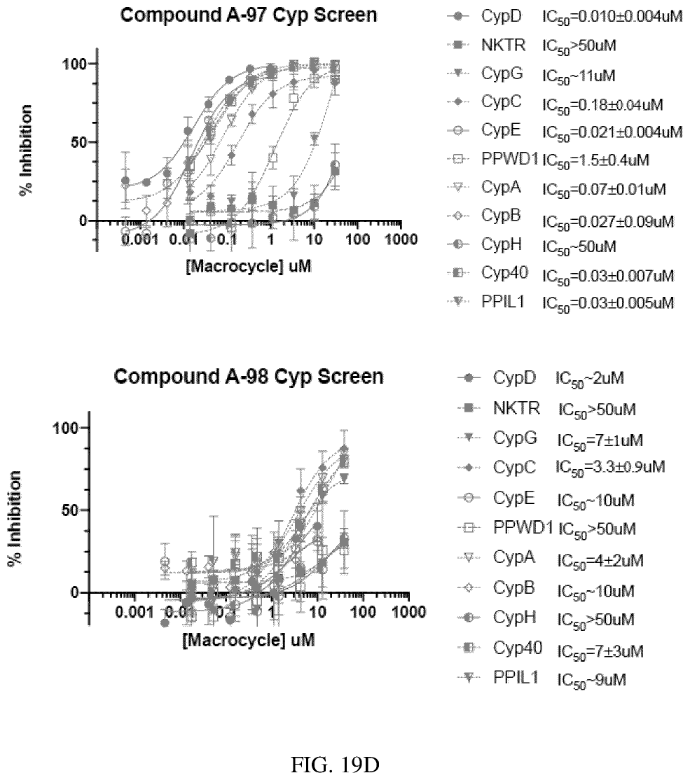

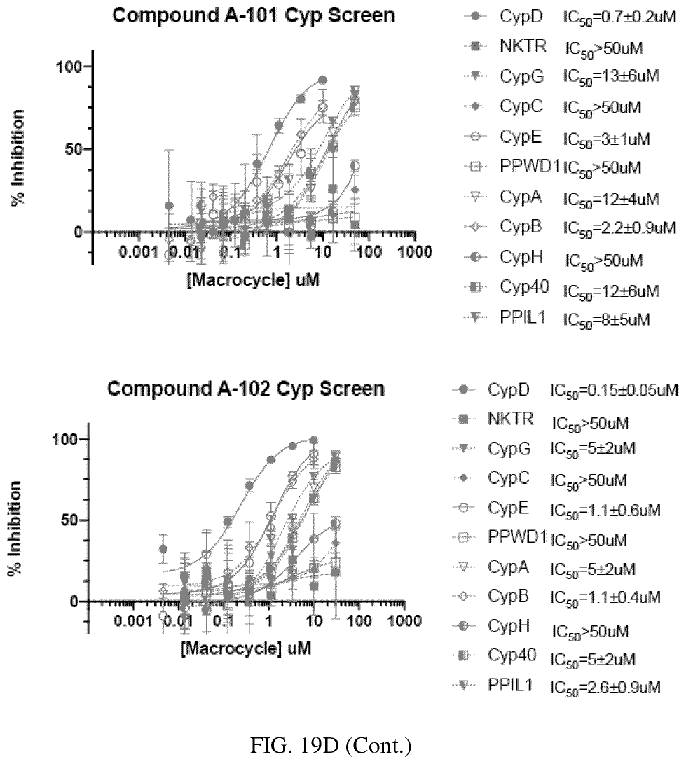

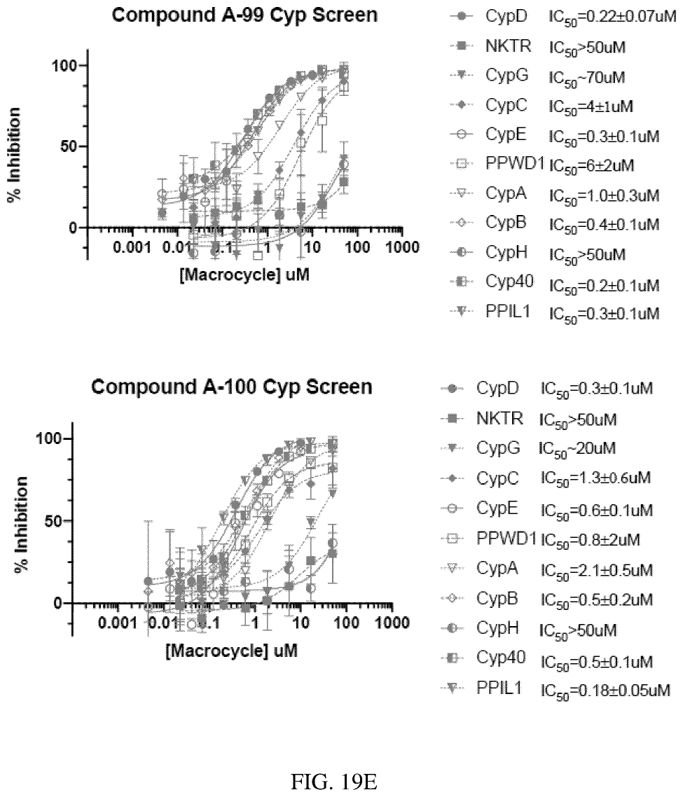

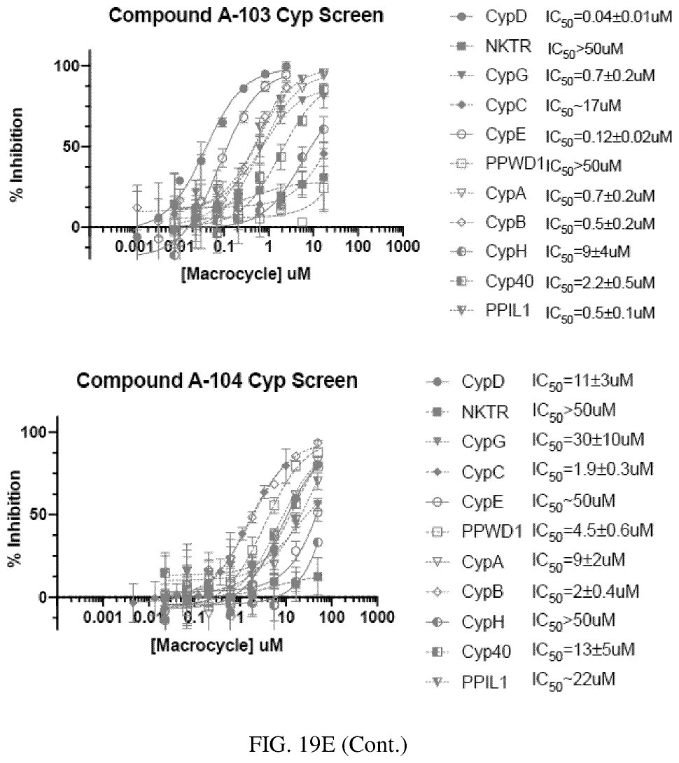

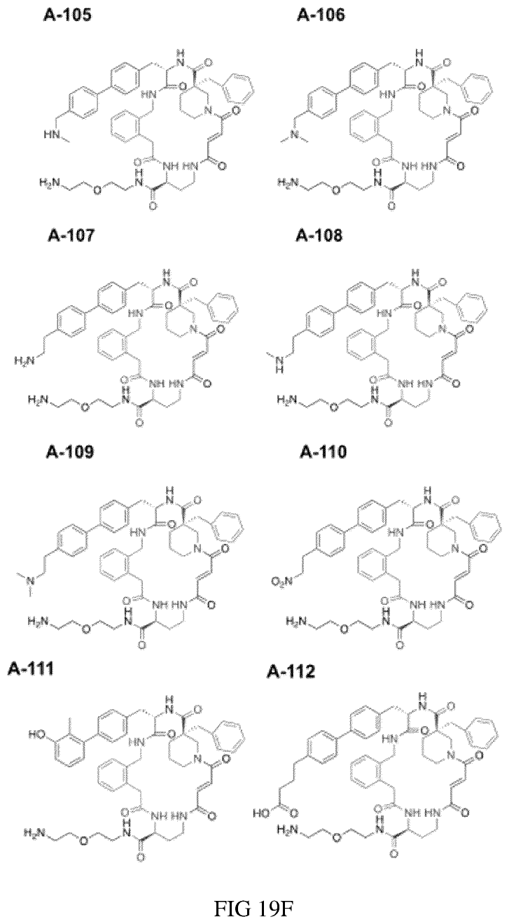

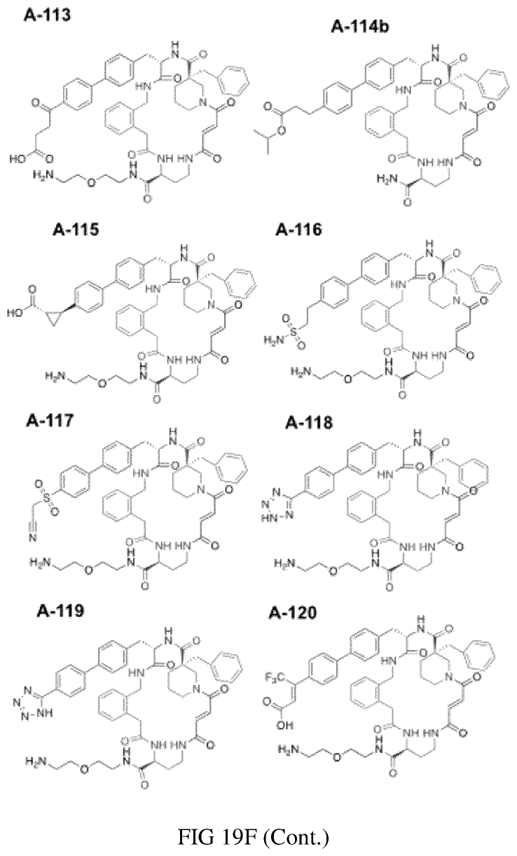

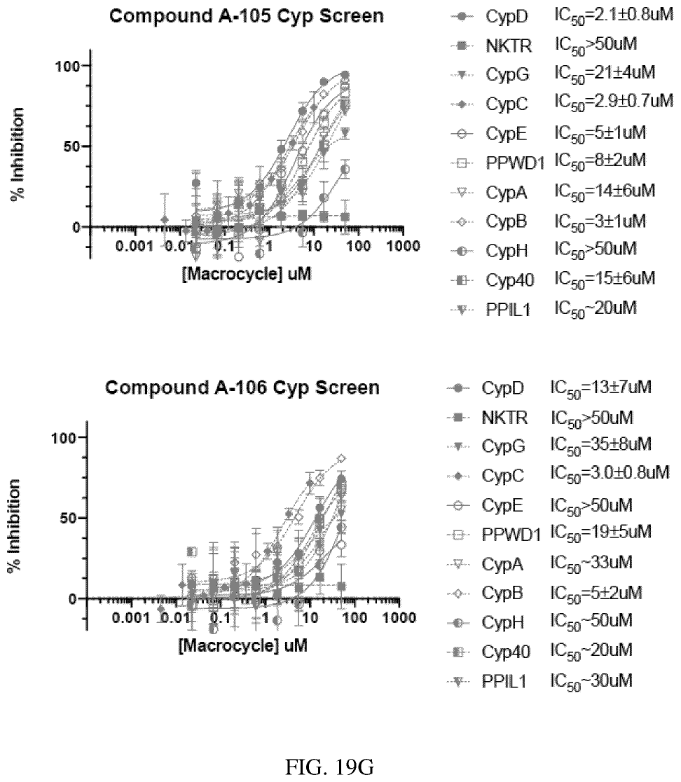

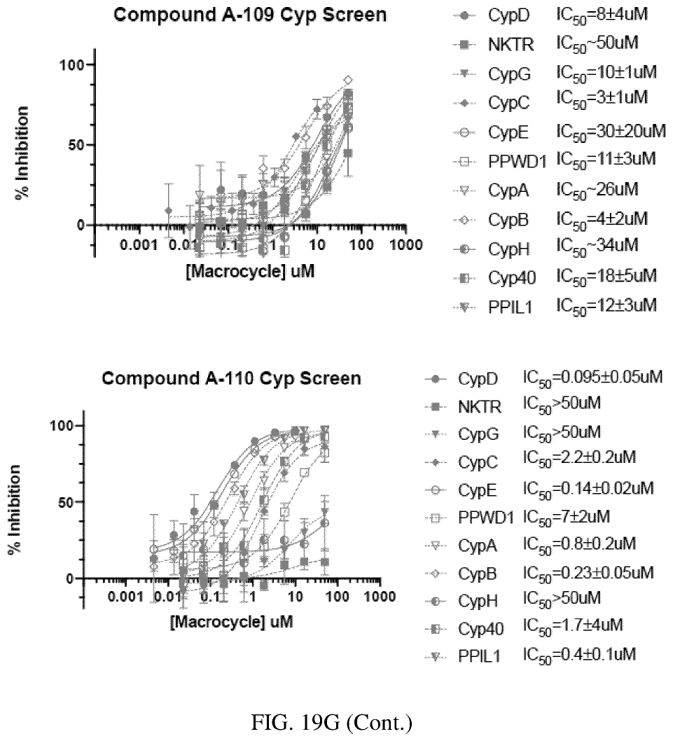

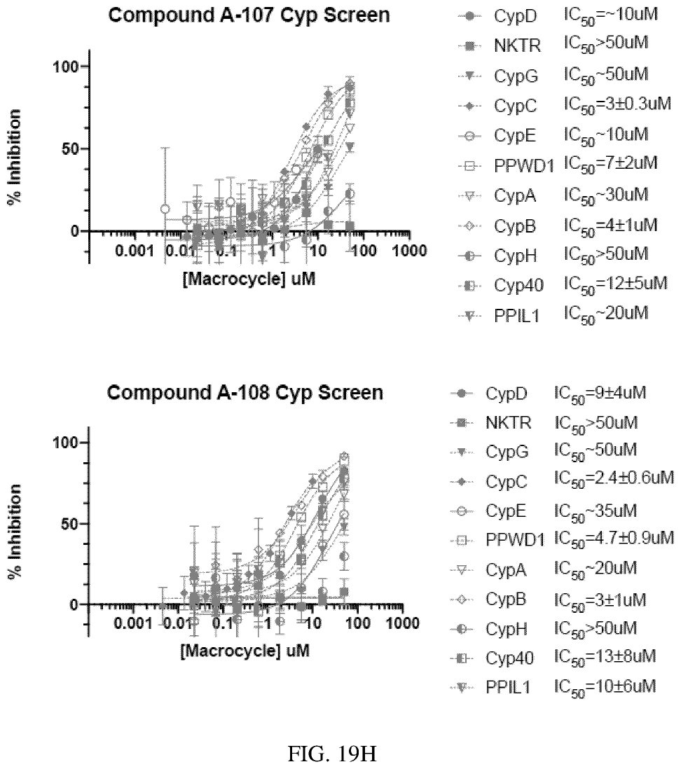

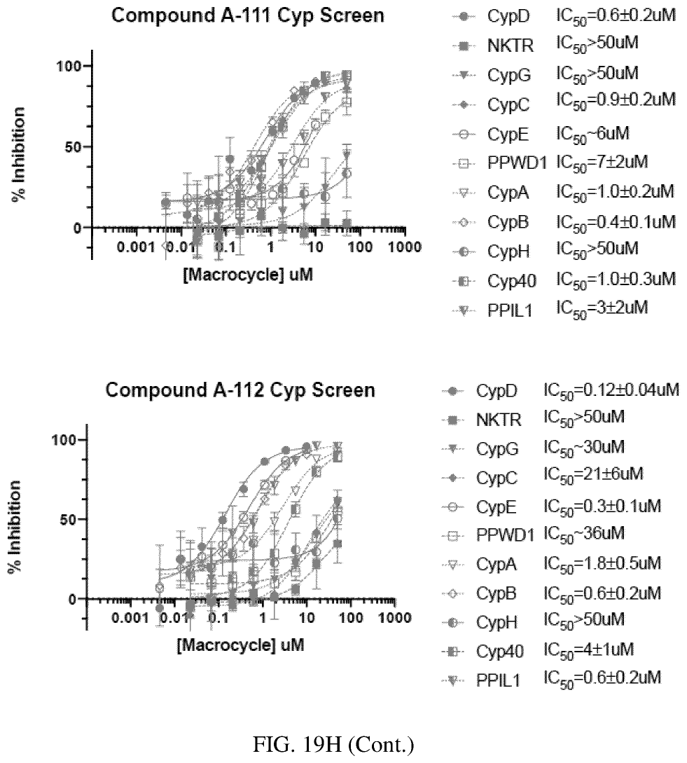

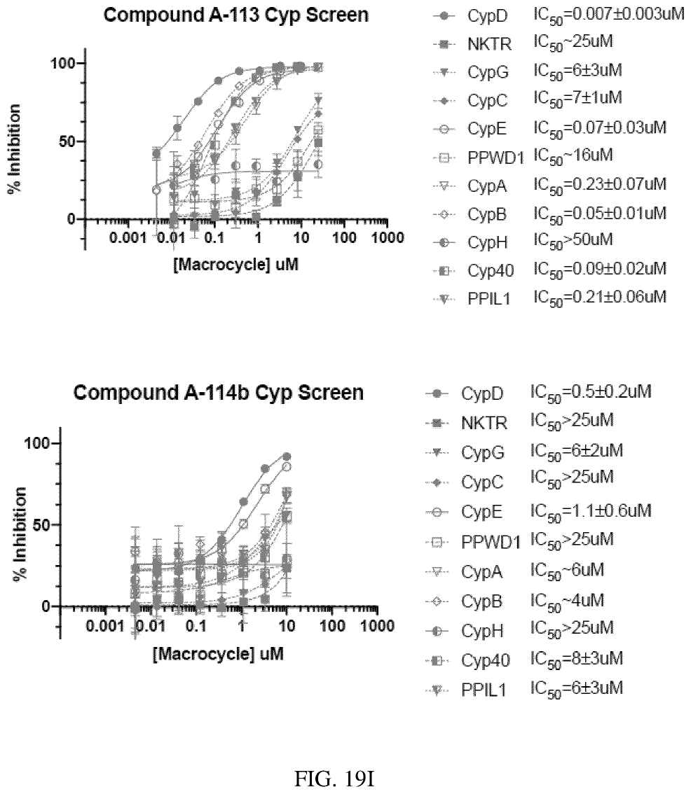

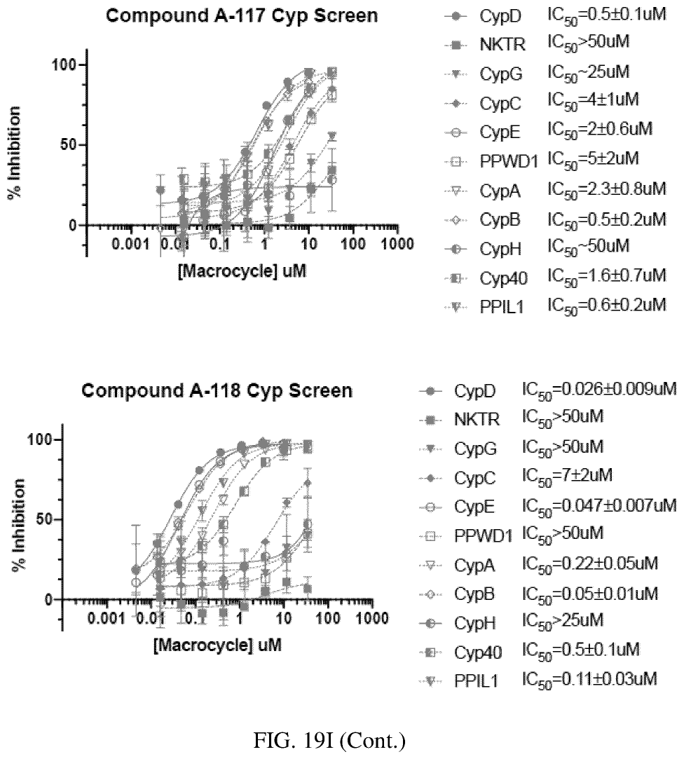

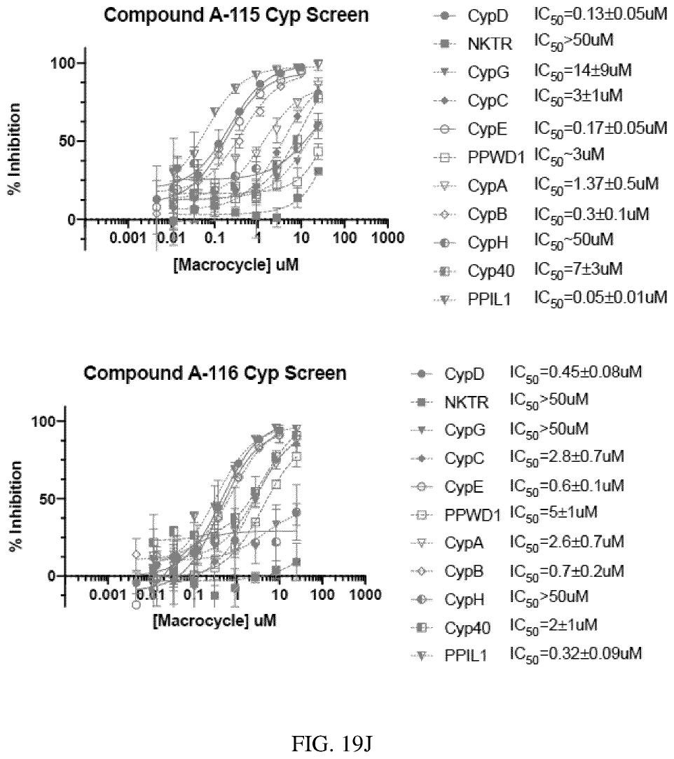

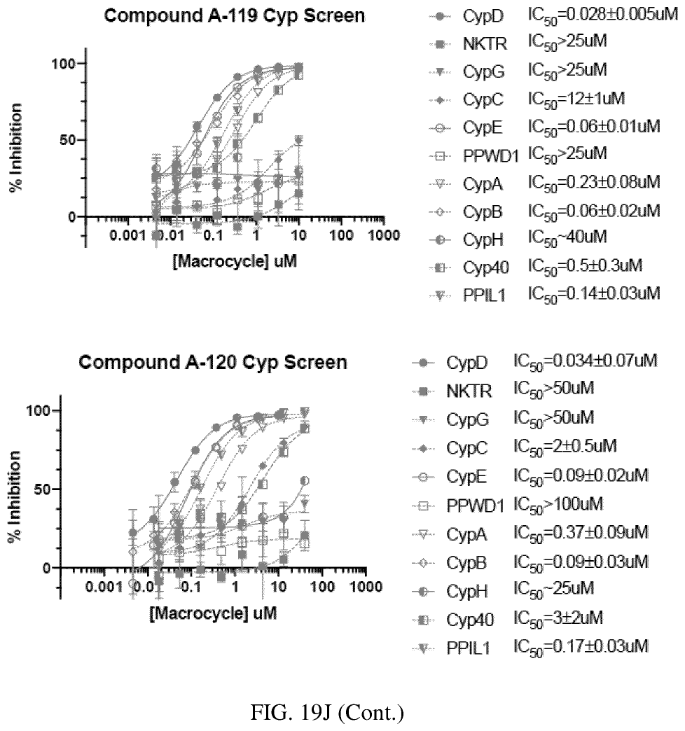

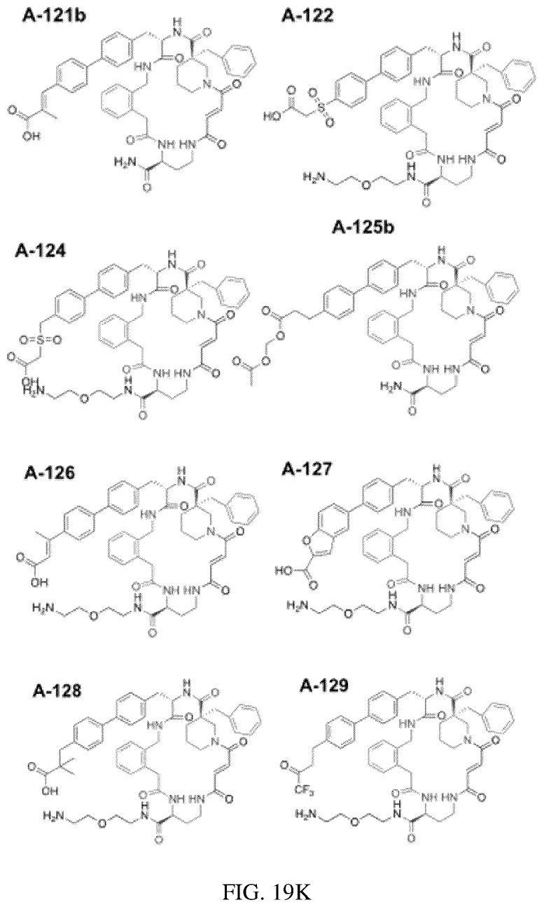

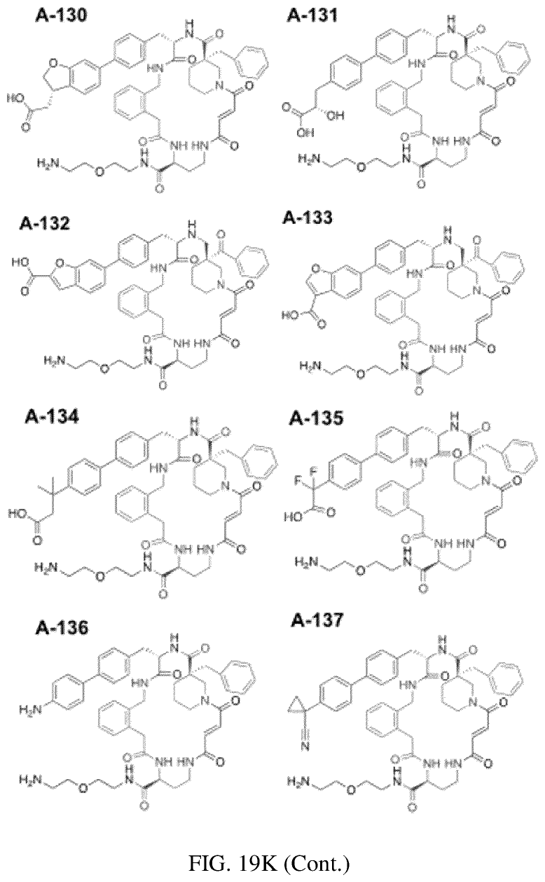

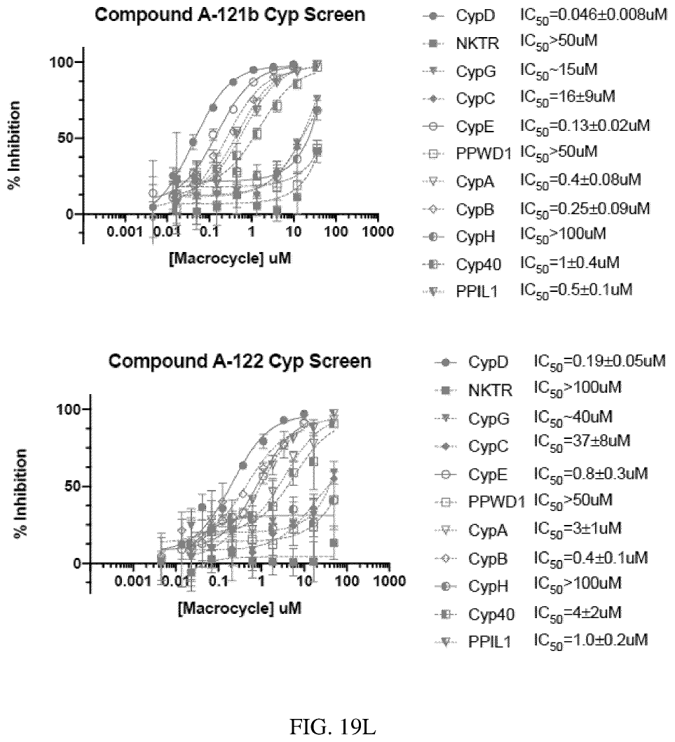

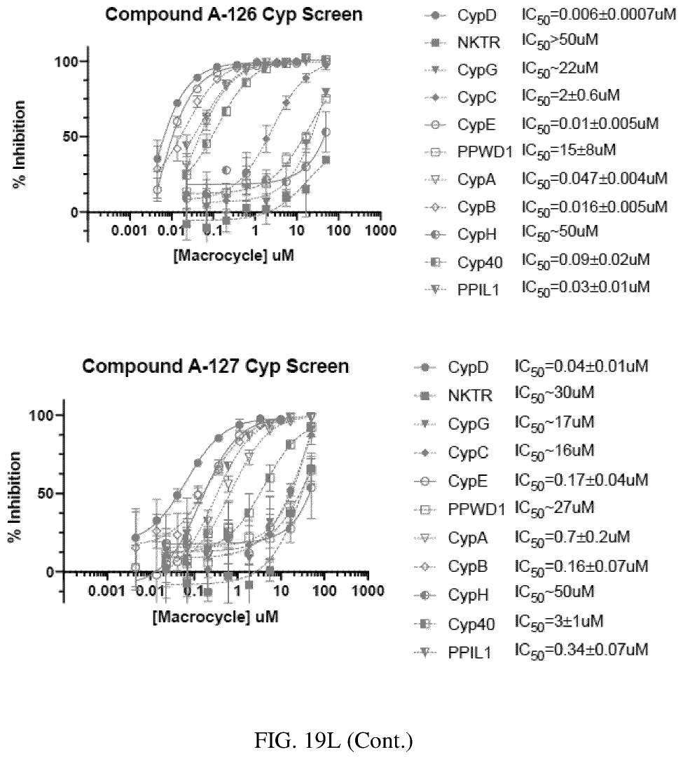

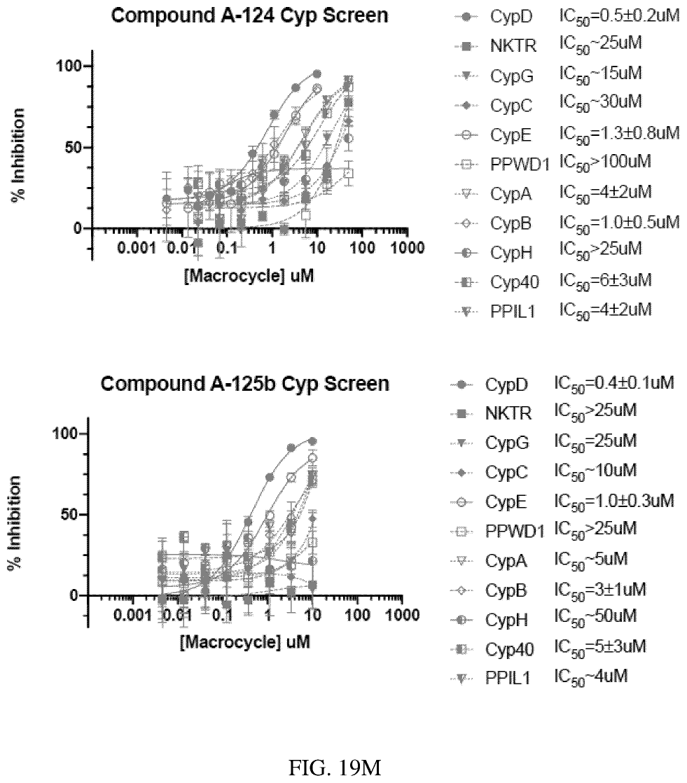

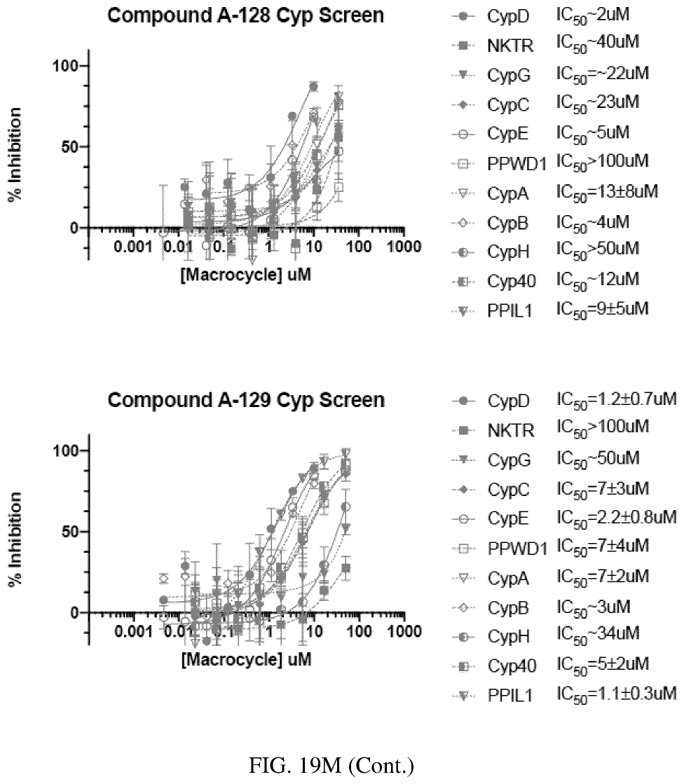

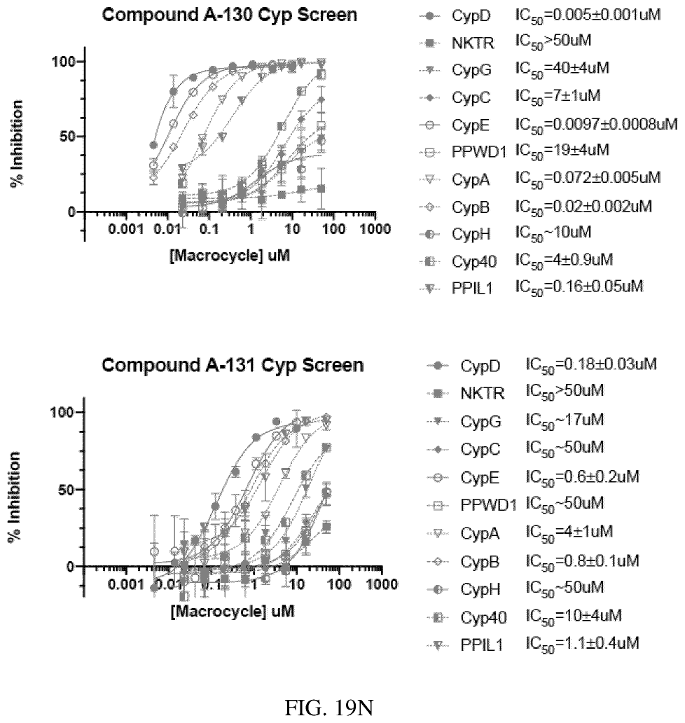

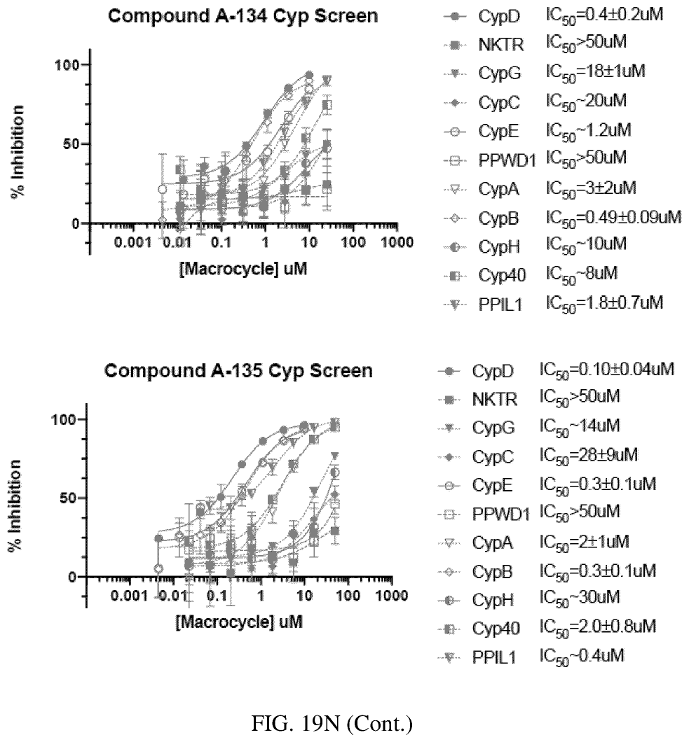

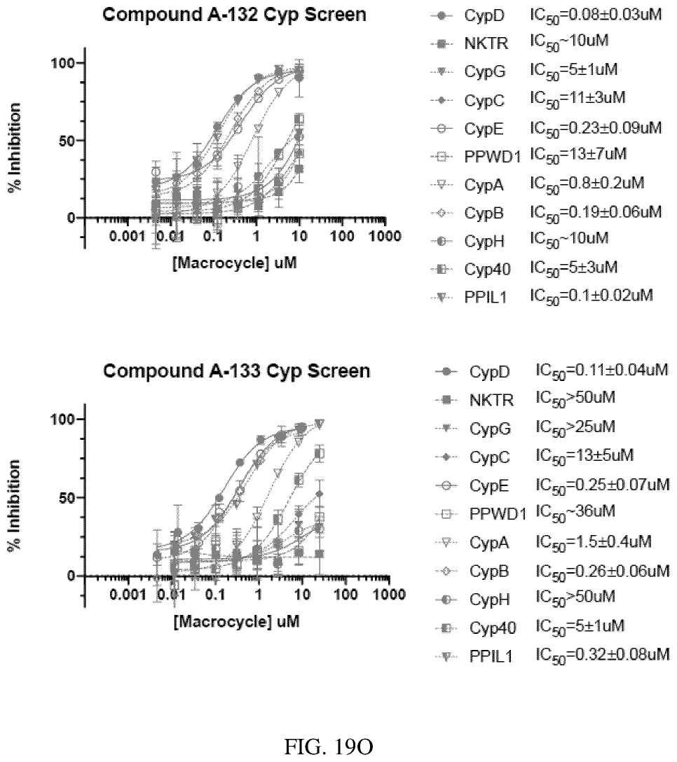

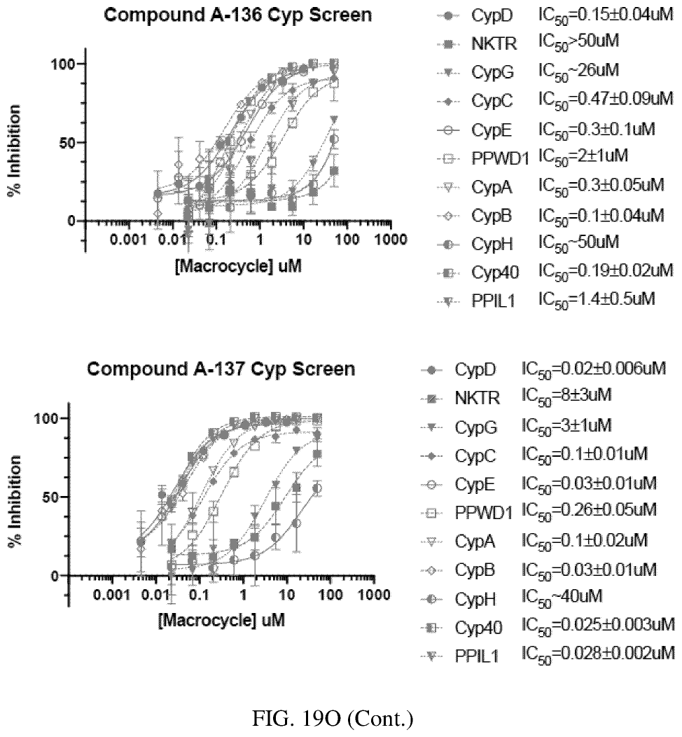

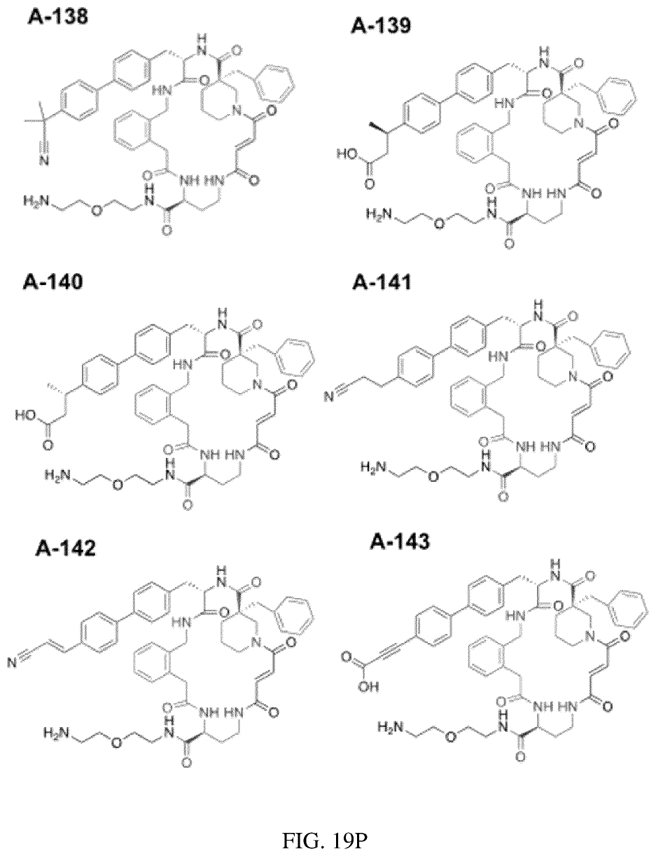

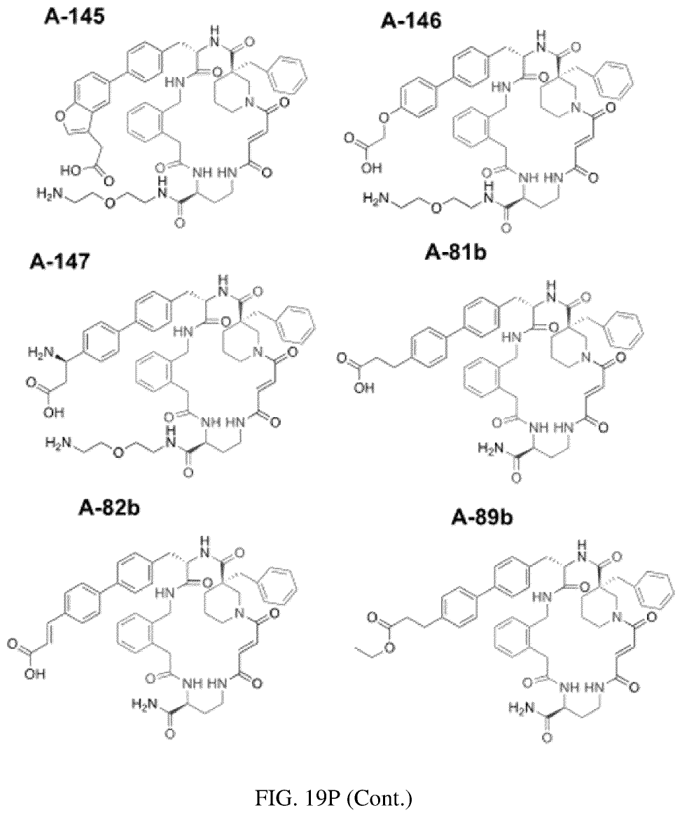

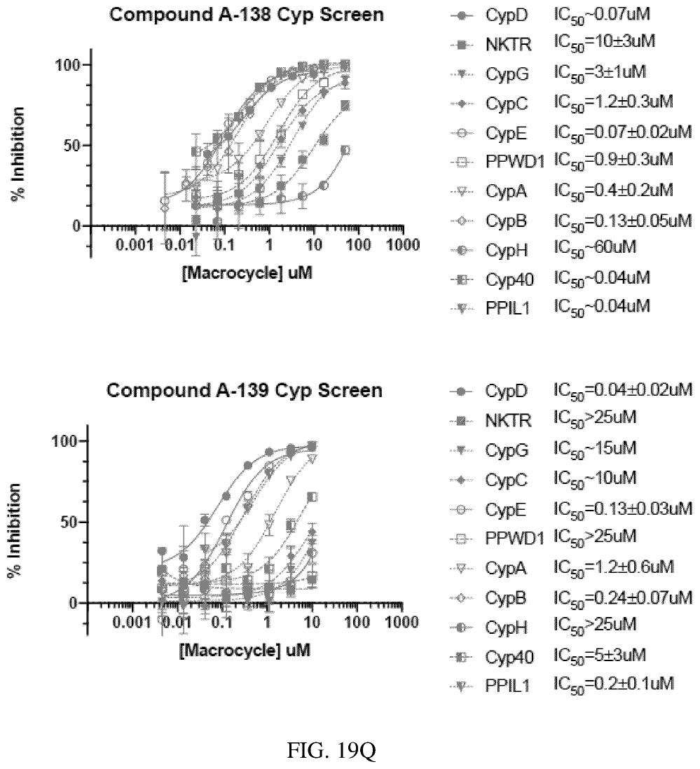

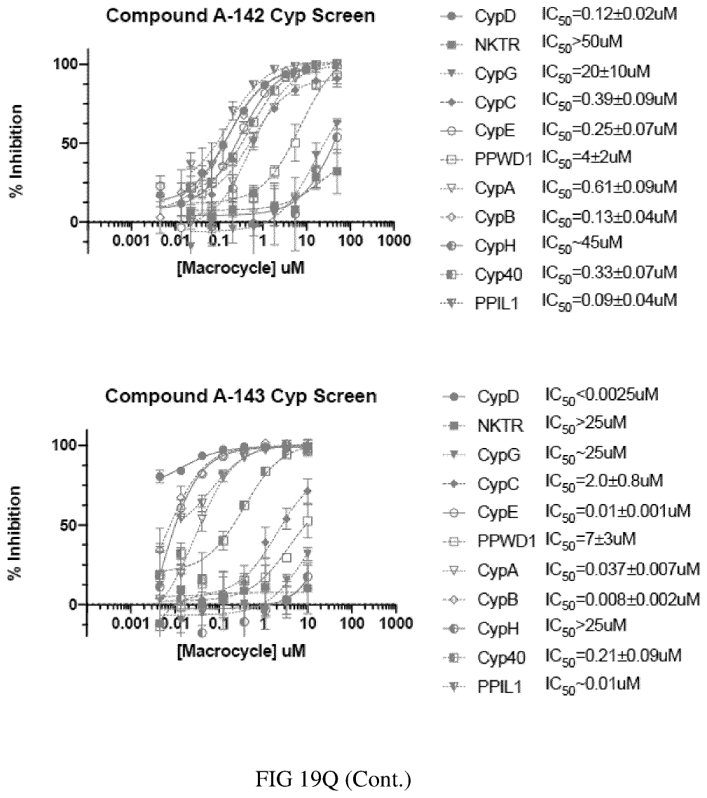

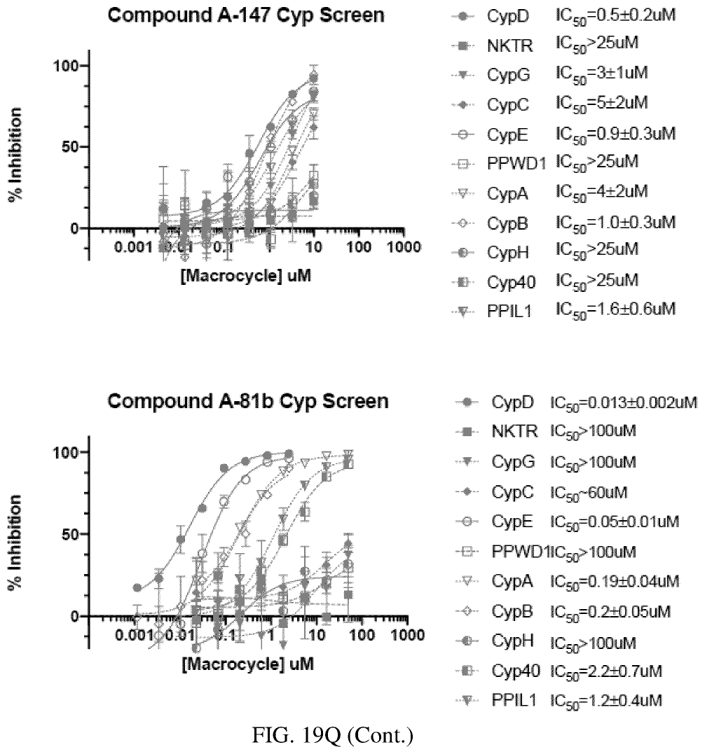

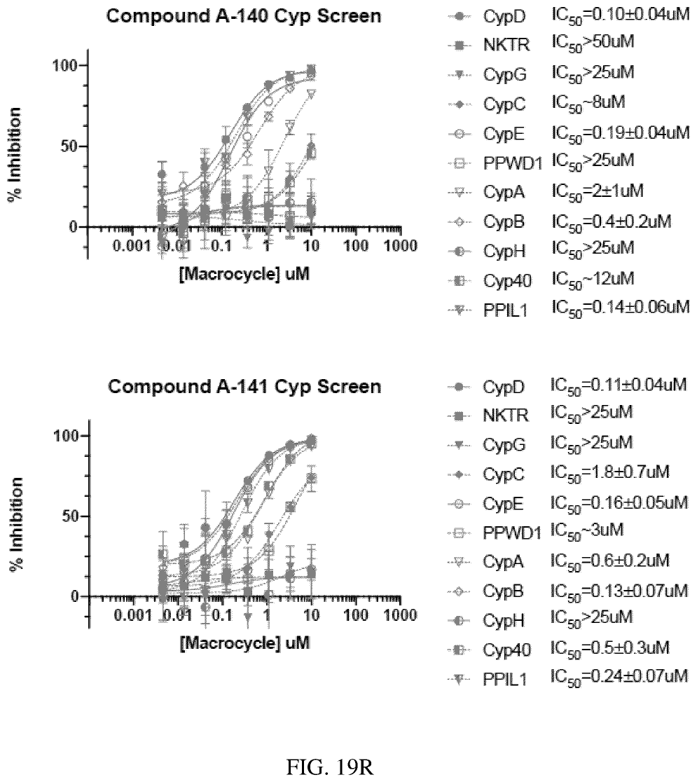

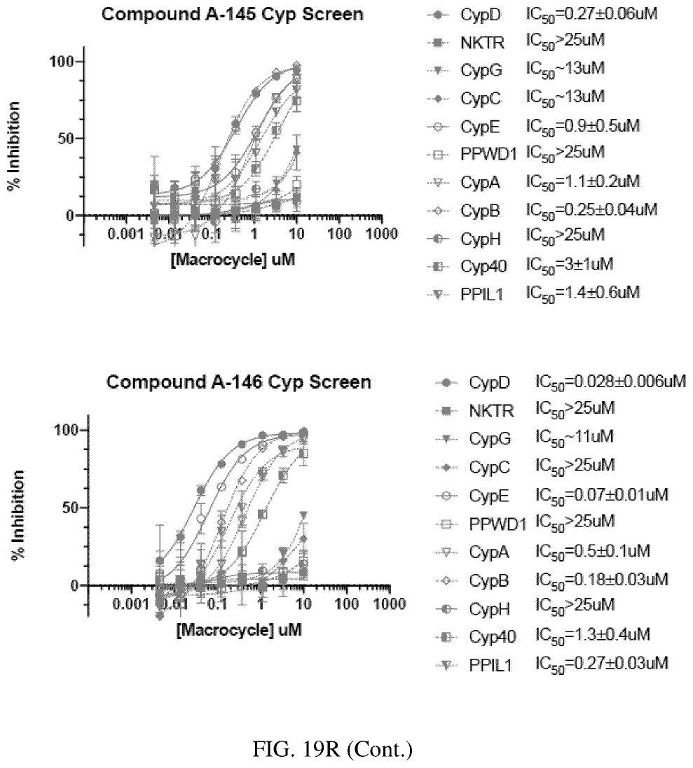

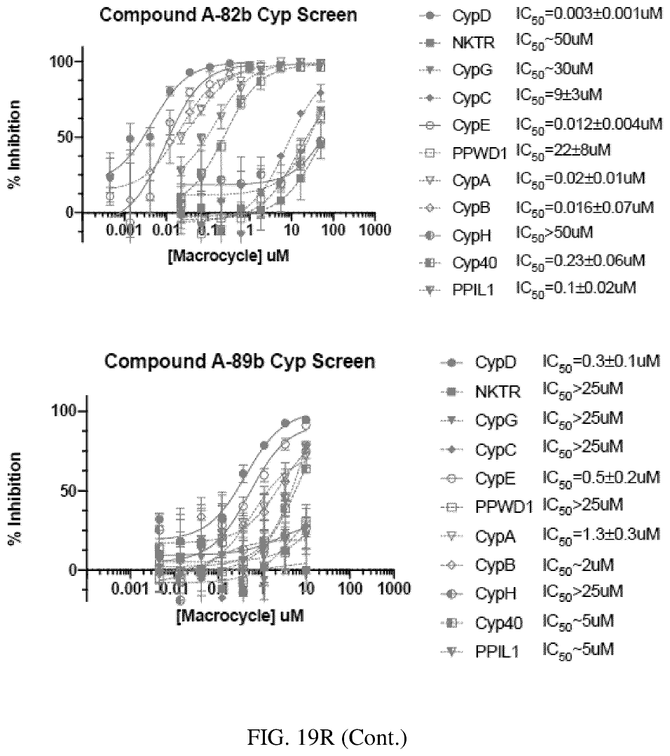

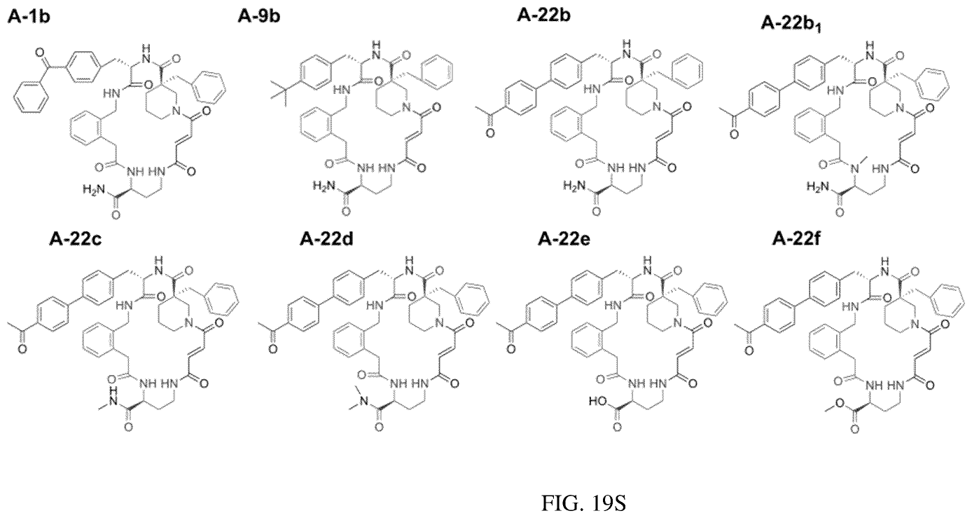

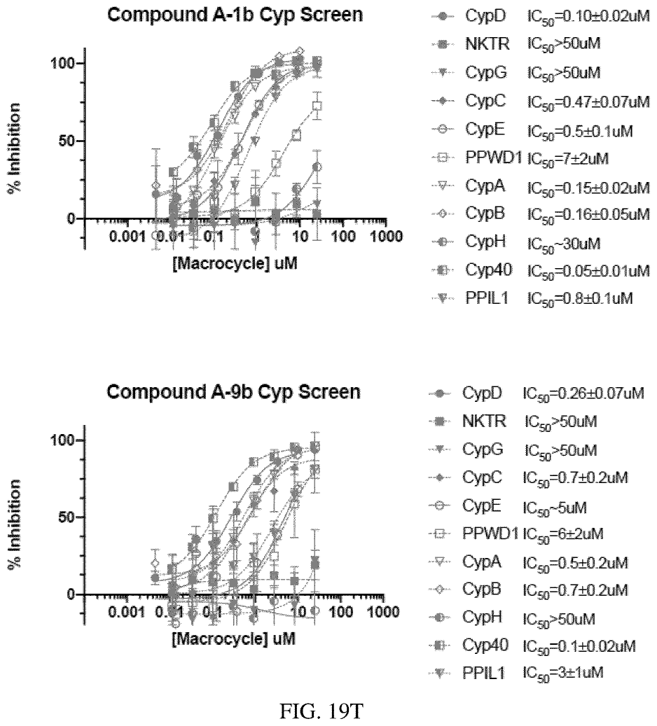

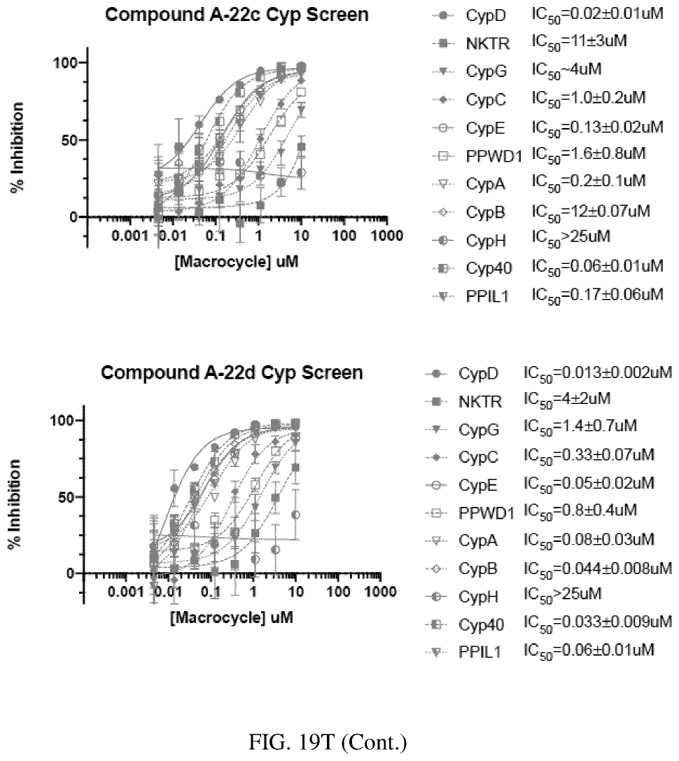

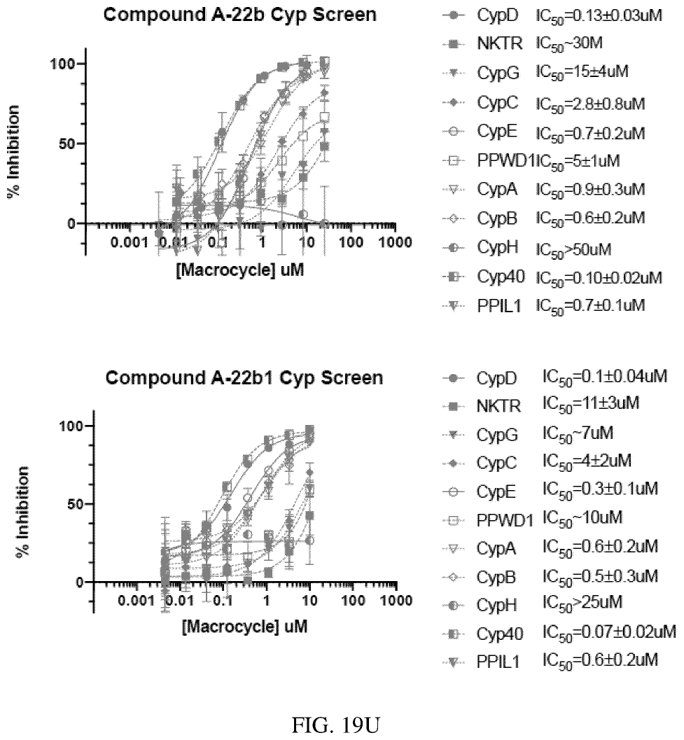

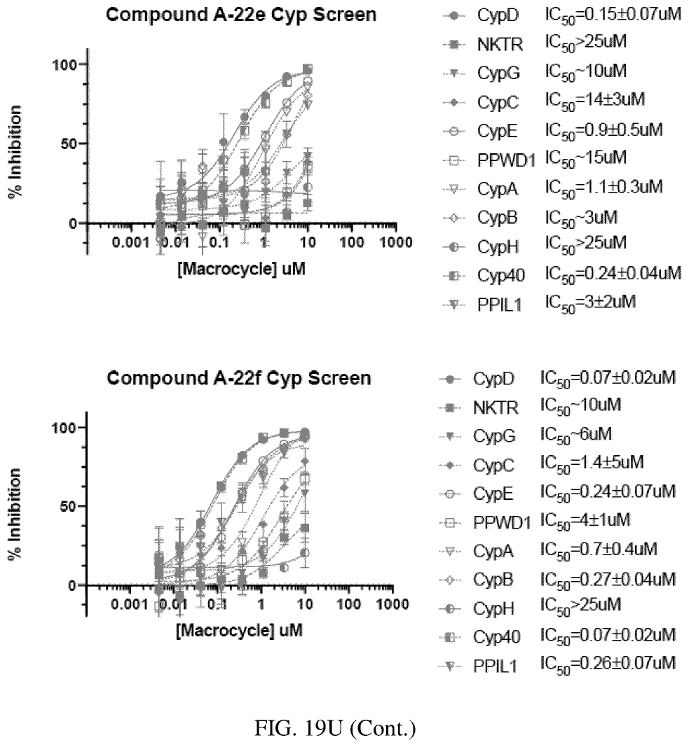

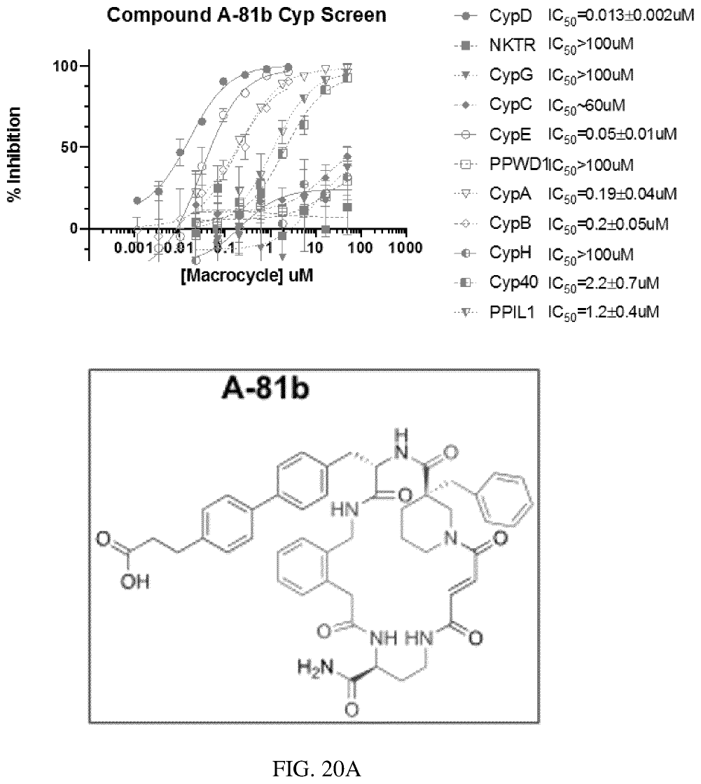

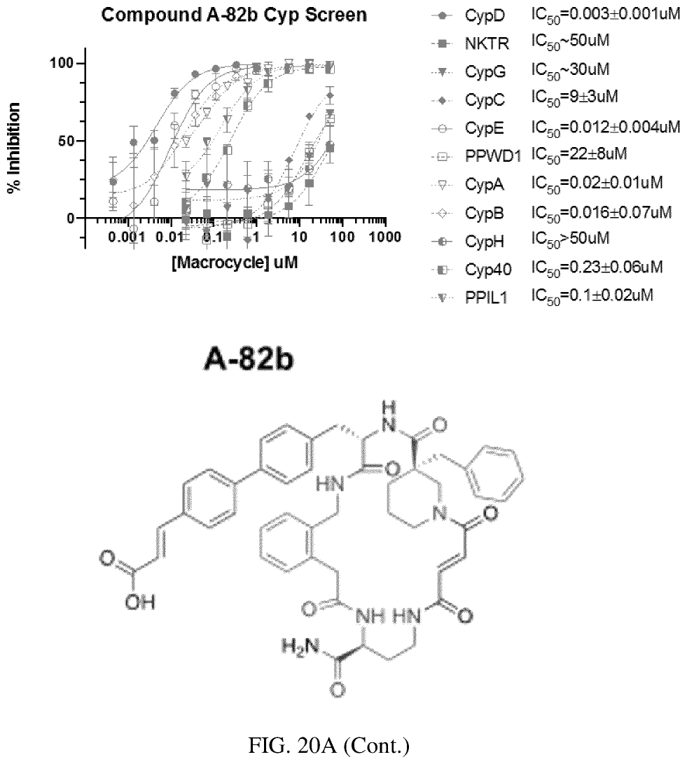

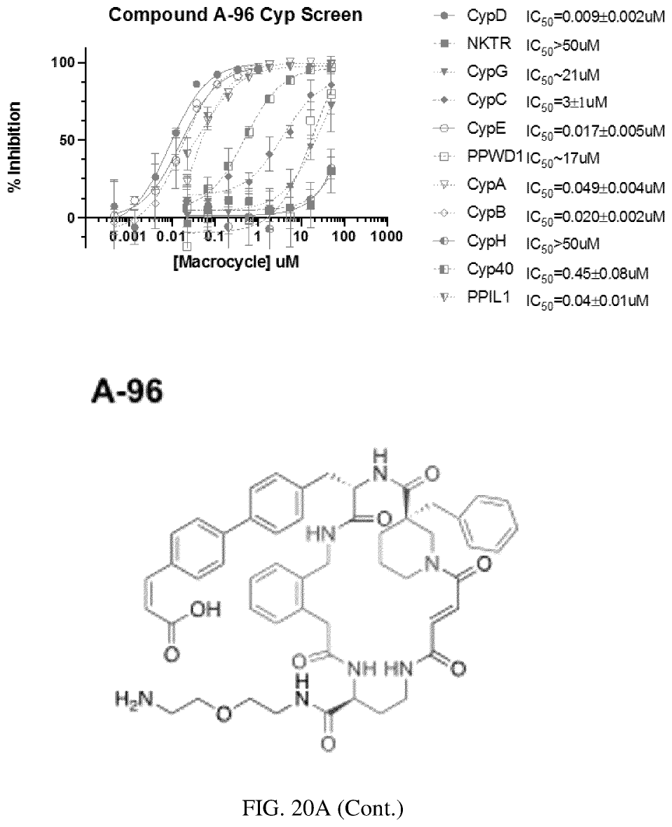

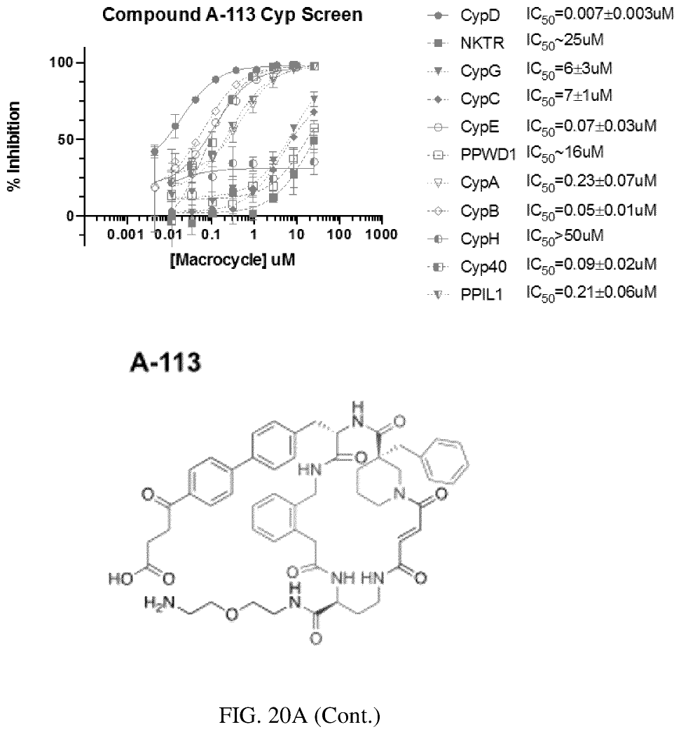

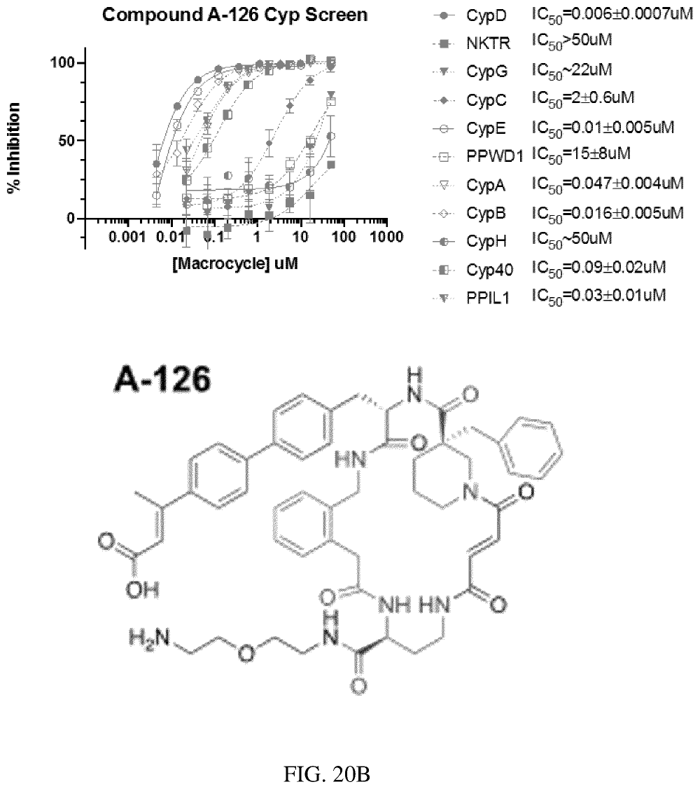

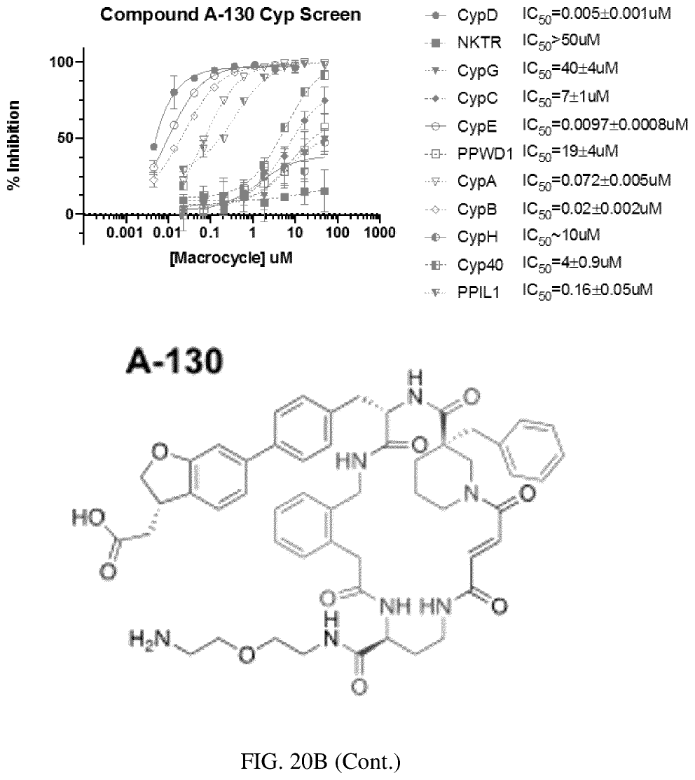

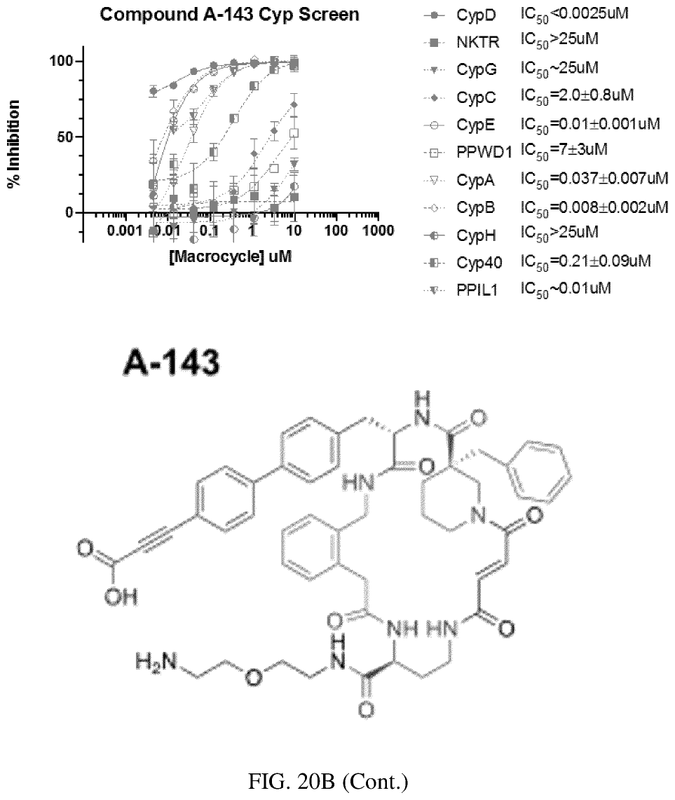

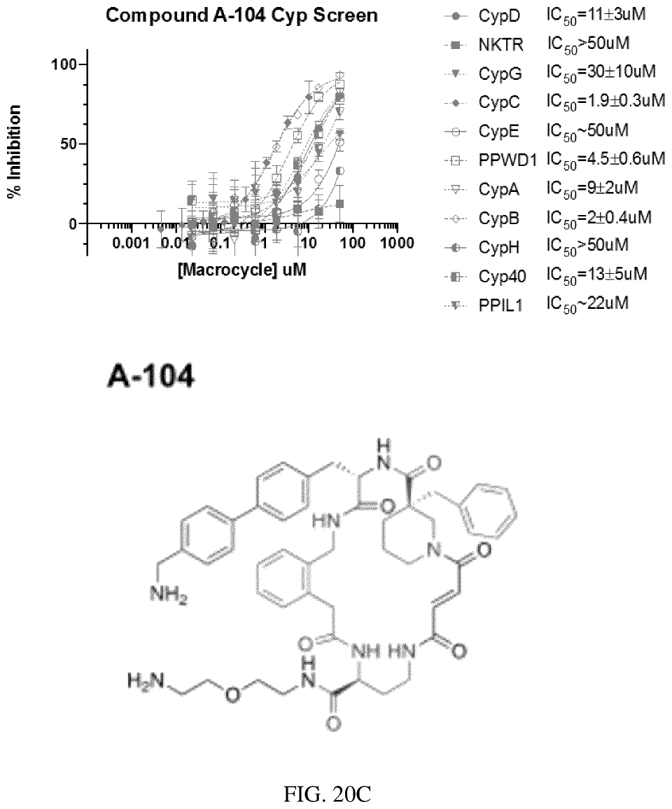

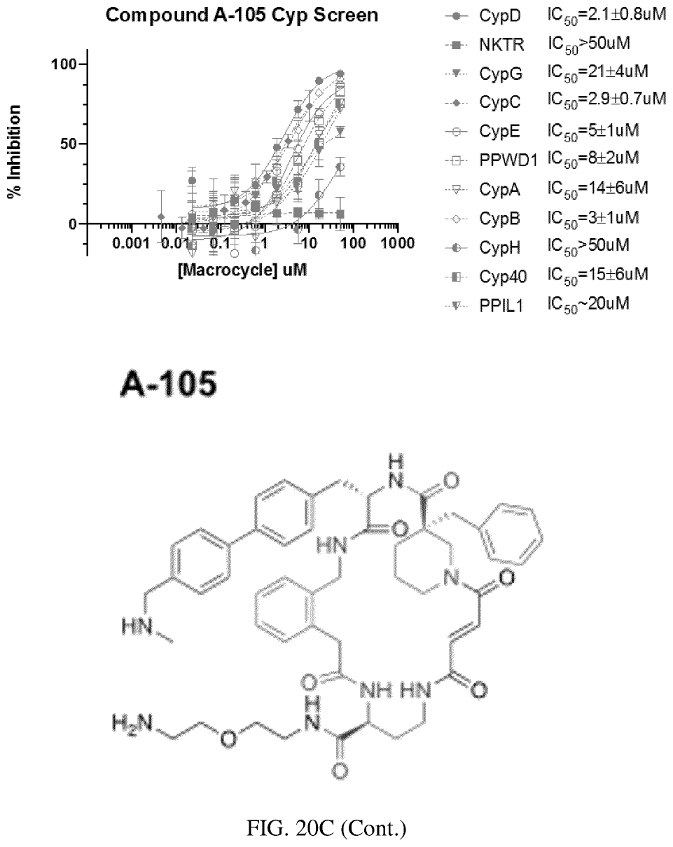

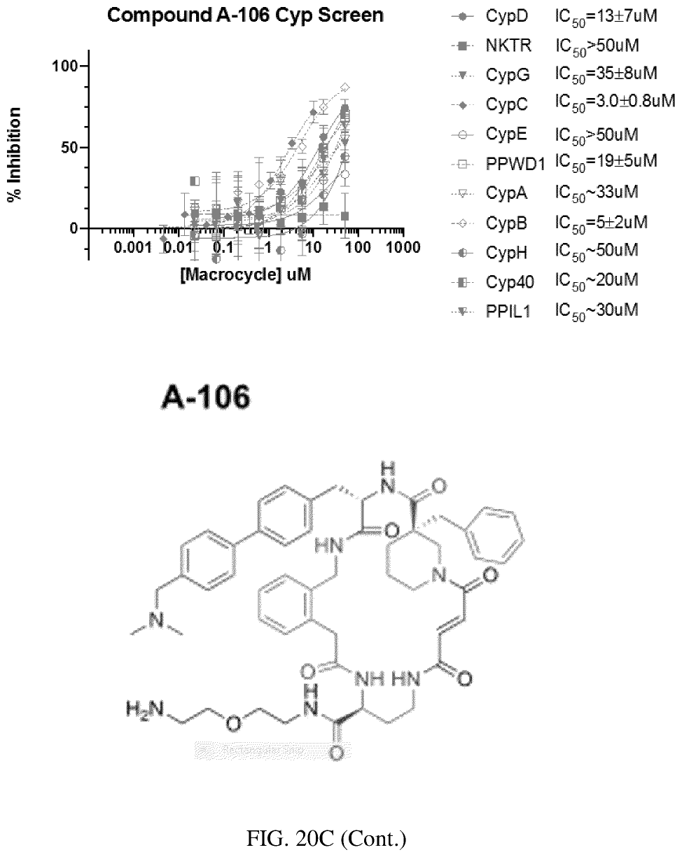

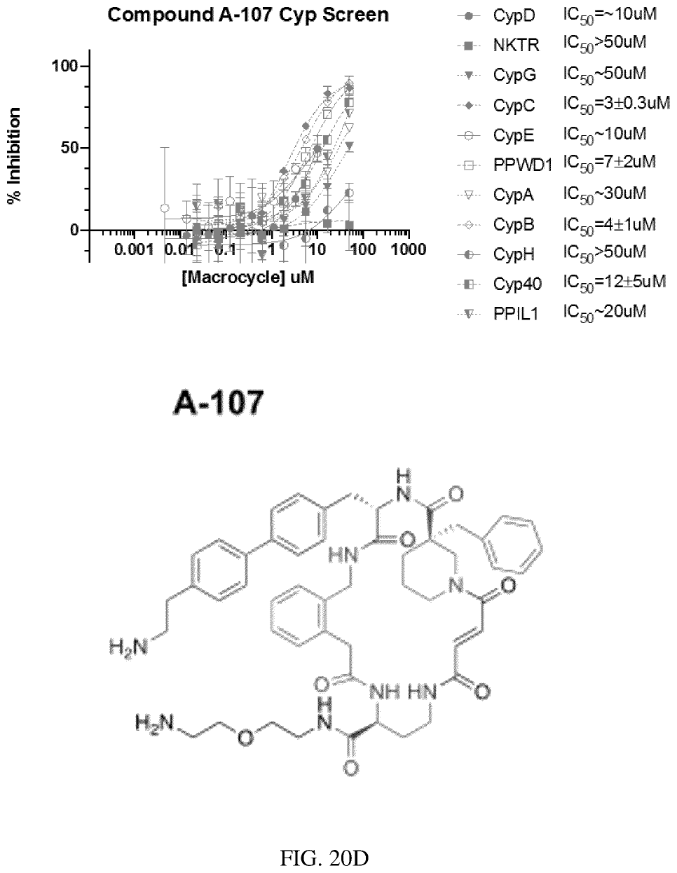

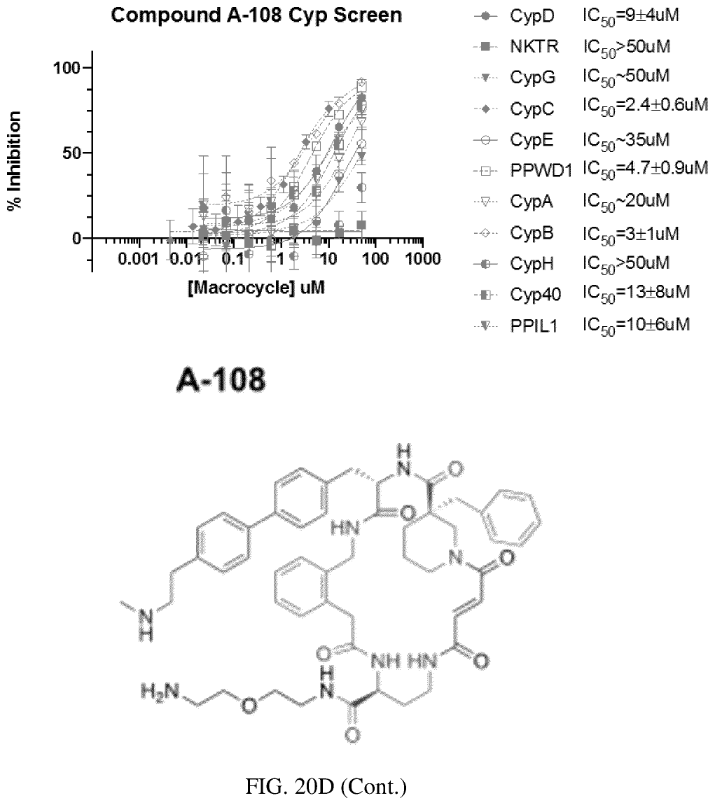

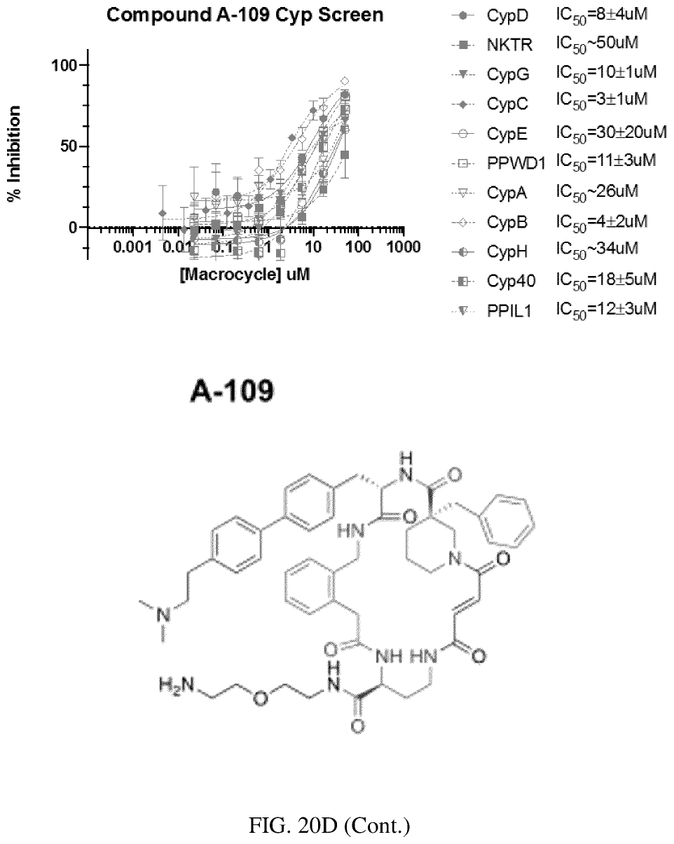

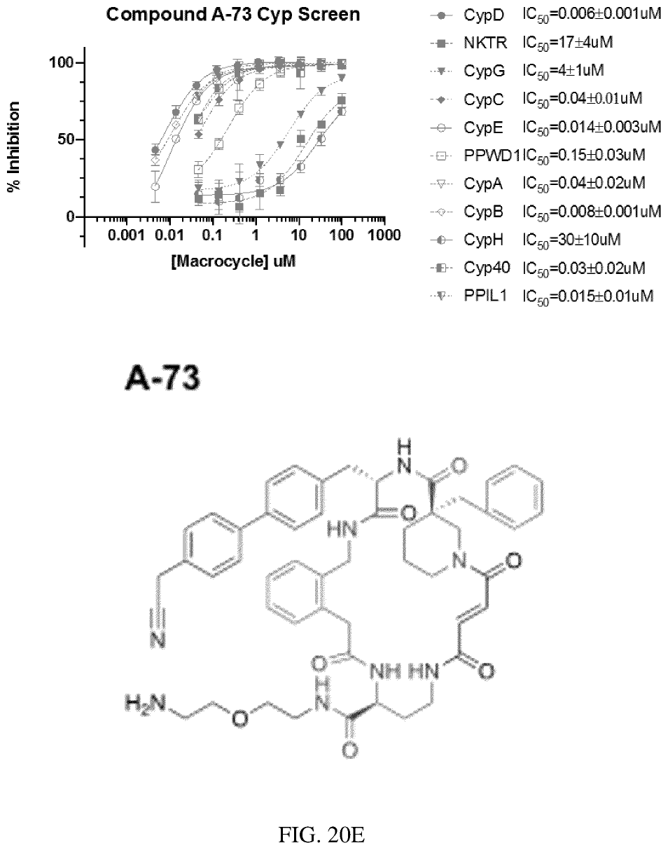

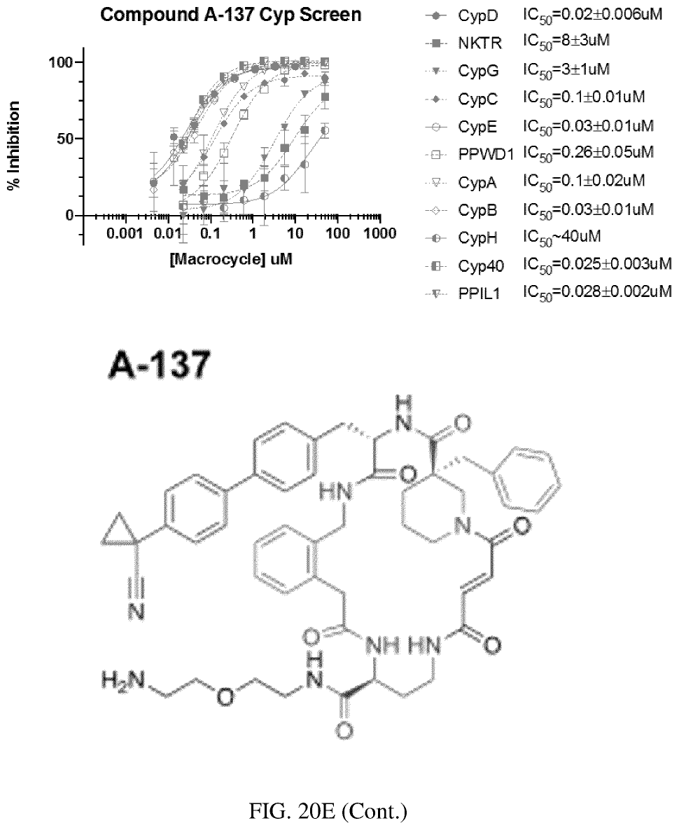

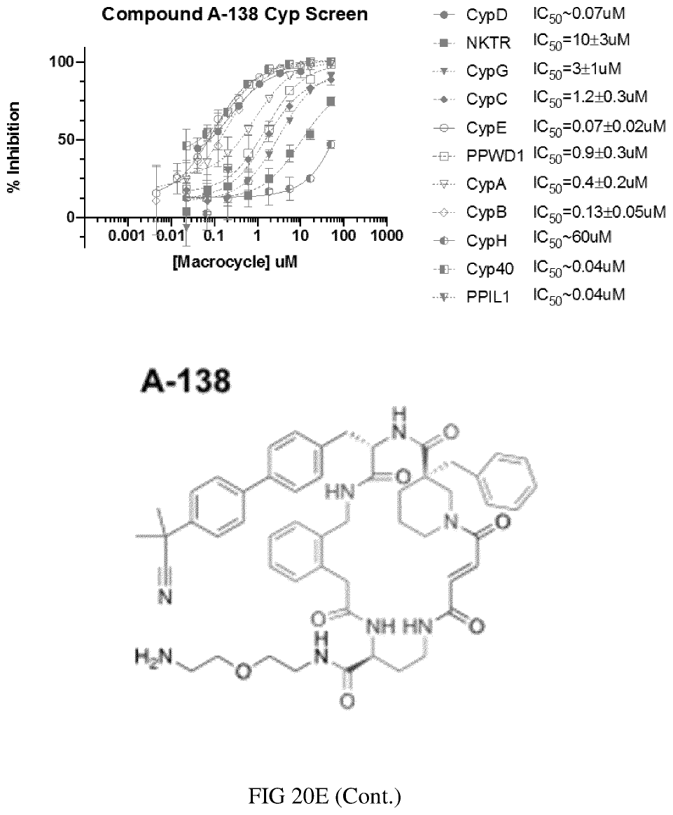

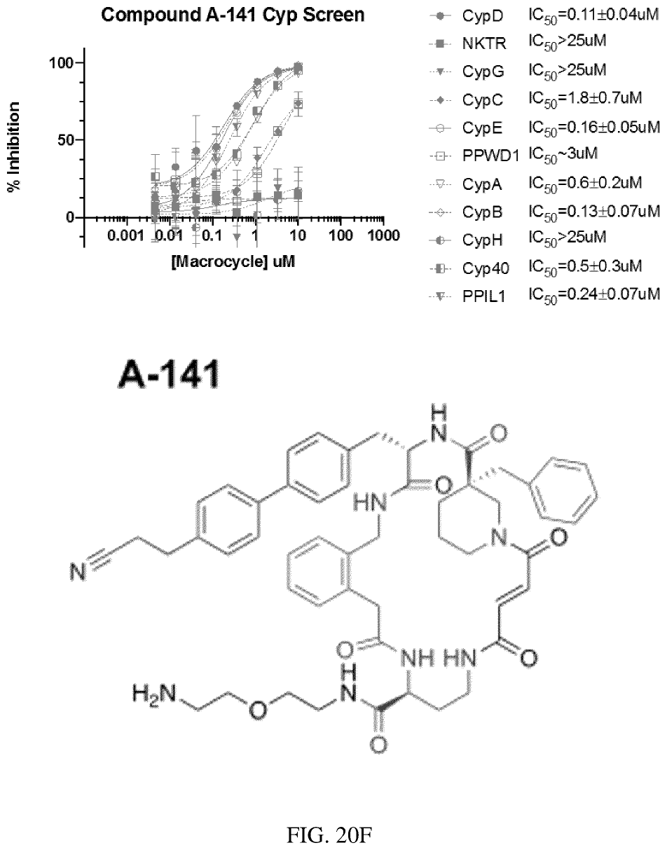

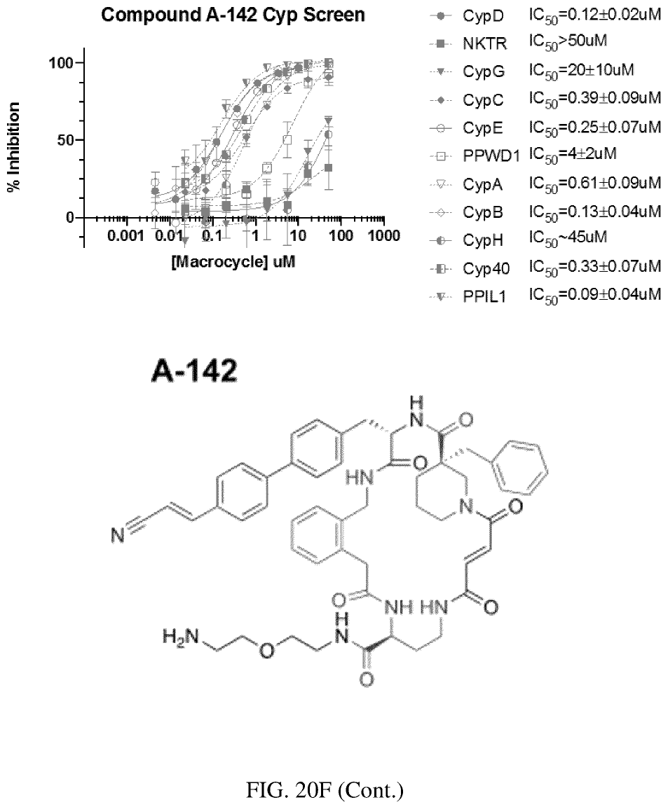

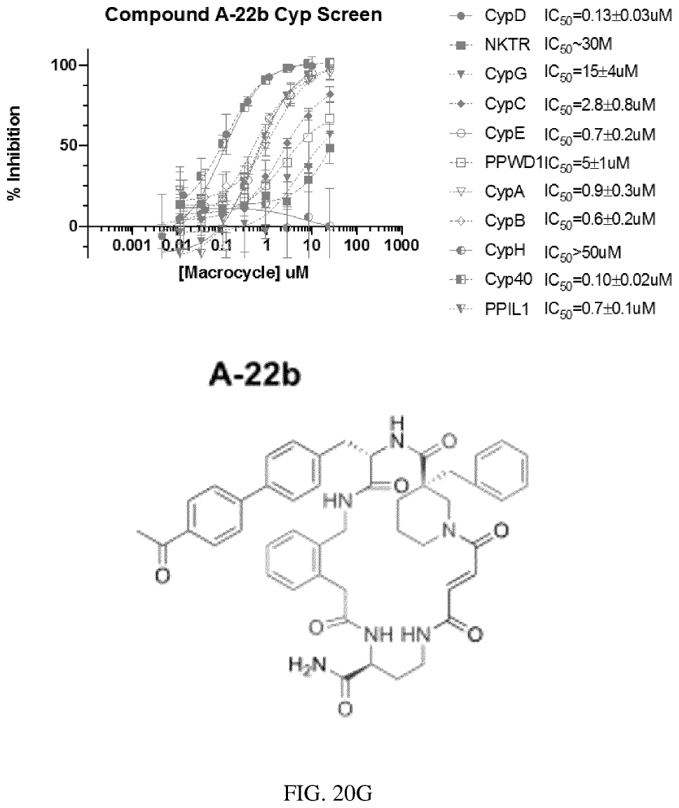

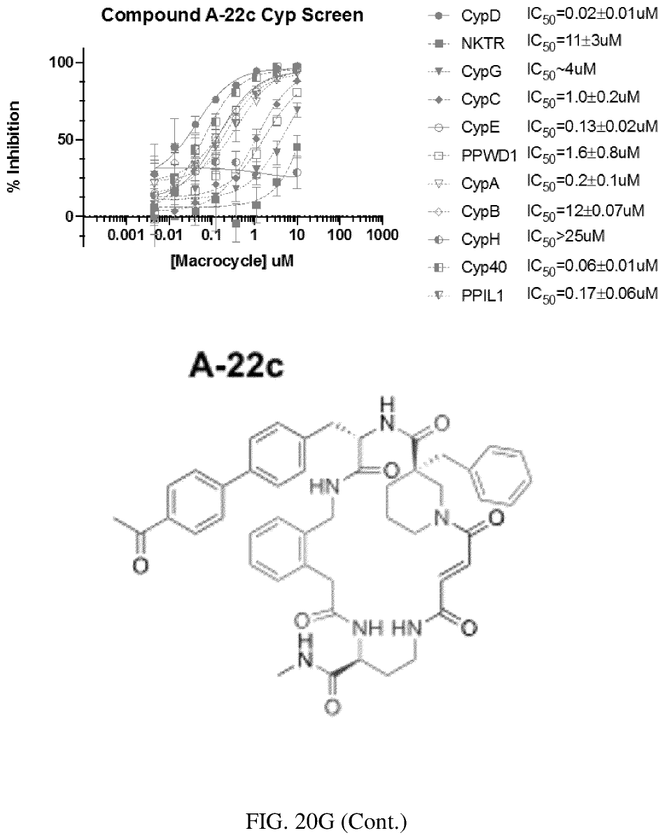

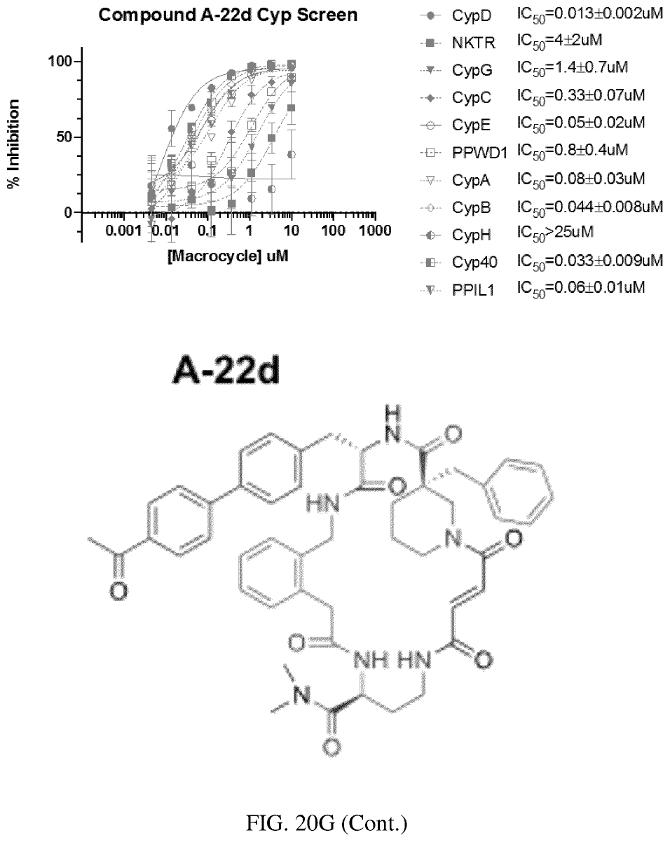

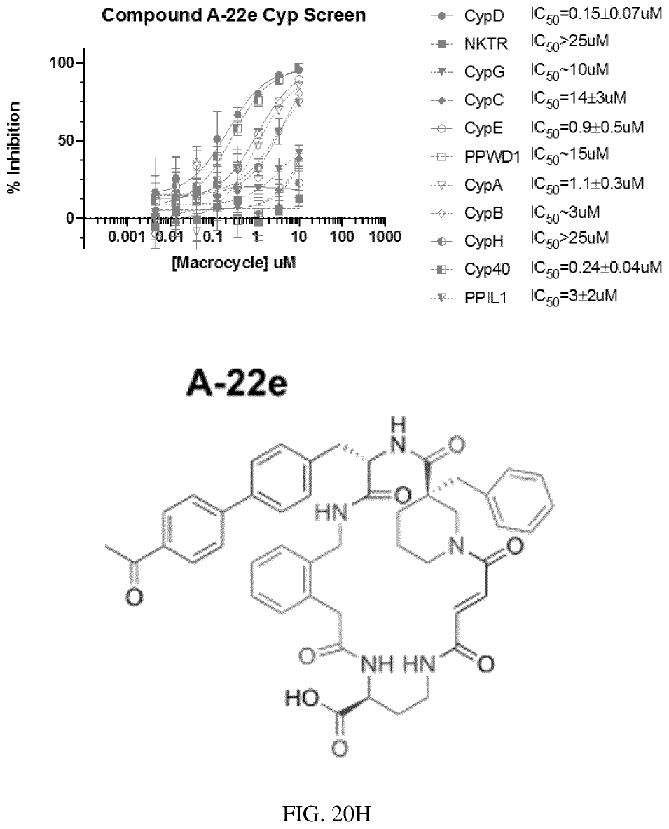

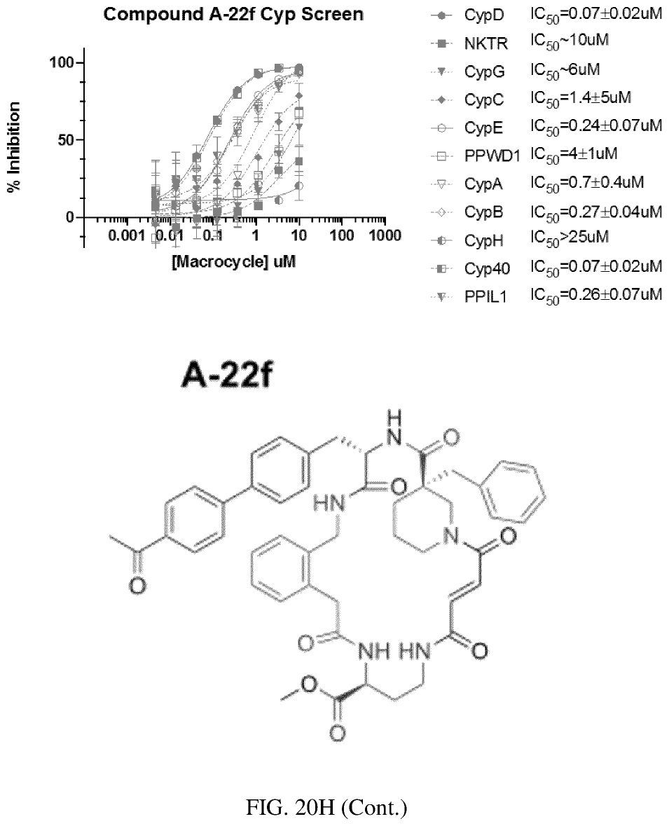

Using conclusions from solved co-crystal structures and specificity profiles, an additional A-series of macrocycles were created (see FIGS. 19A-19U). Further evidence was found showing that the carboxylate inhibitors engender CypD selectivity and high potency (see FIGS. 20A-20B). It was determined that placement of the carboxylate is important for potency. It was further determined that conversion of the carboxylate to amine appears to eliminate potency and shift selectivity, where a (−) charge on carboxylate appears to engender specificity and potency, and a (+) charge on amine flips selectivity to CypC/PPWD1/CypB, albeit at lower potency (see FIGS. 20C-20D). Nitriles provide similar potency, but have reduced selectivity for CypD, and just like carboxylates, need optimal geometry and placement (see FIGS. 20E-20F). The core compound scaffold can still support a variety of moieties on 4th building block tail, the site of DNA attachment in the compound library (see FIGS. 20G-20H).

### Co-Crystal Structure of A-81 Bound to CypD

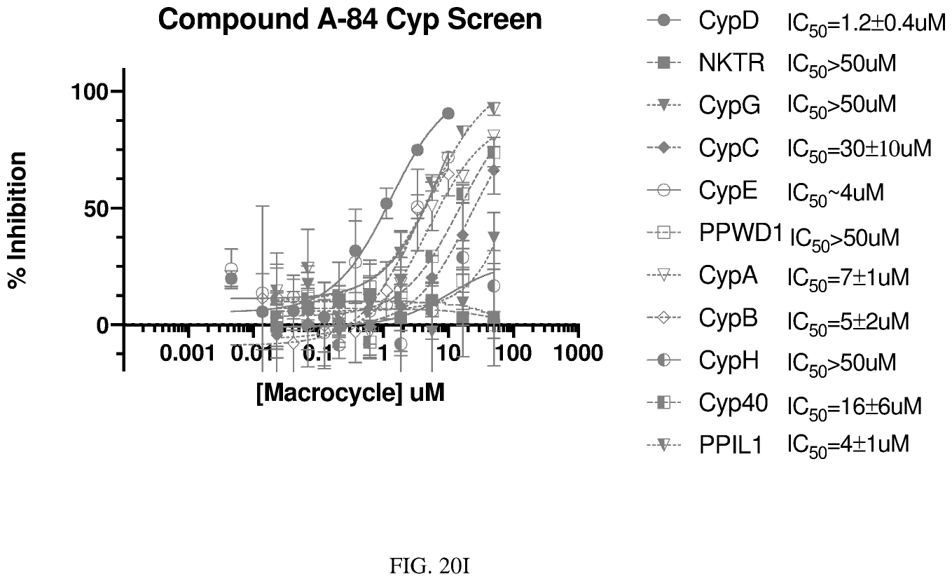

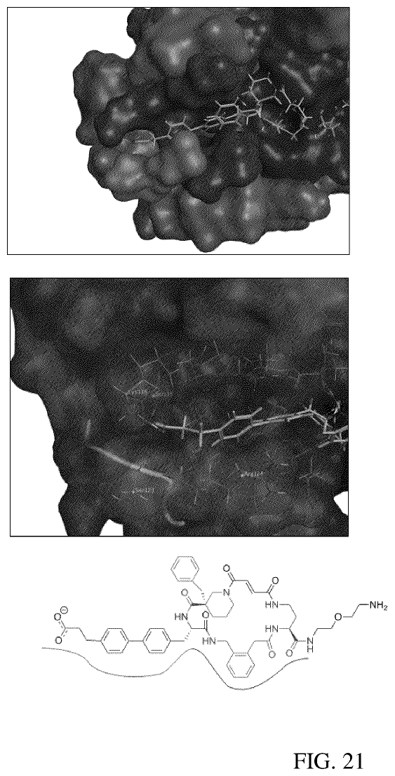

The co-crystal structure of A-81 maintains the same interactions as the previous crystal structure scaffolds in active site and S2 pocket (see FIG. 21). There are H-bonds with non-gatekeeper Ser119, and a salt bridge with non-gatekeeper Lys118.

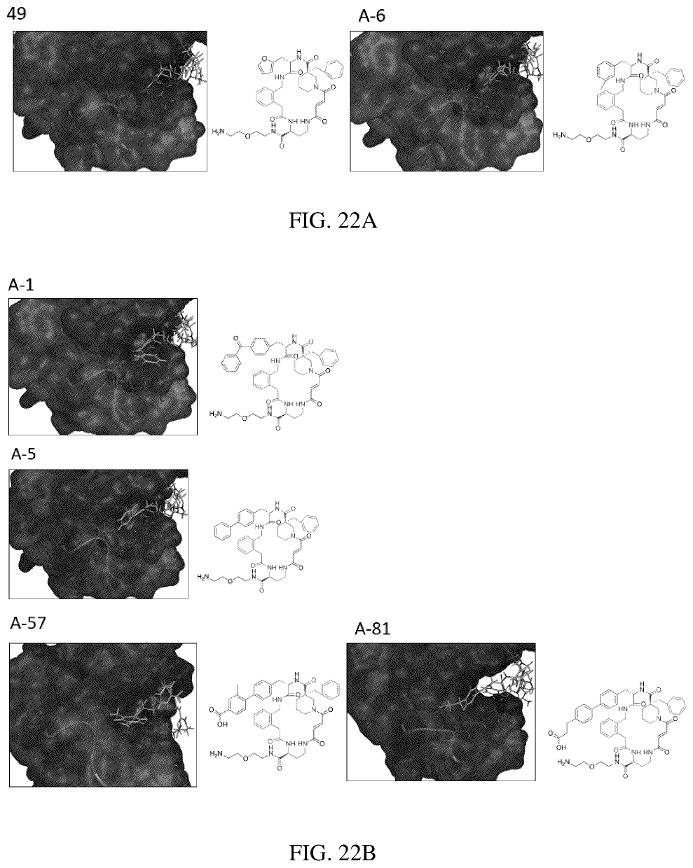

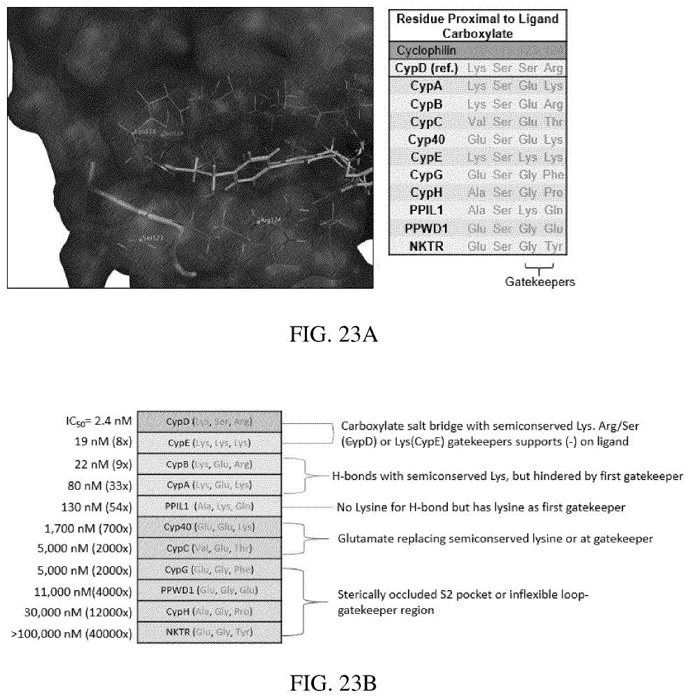

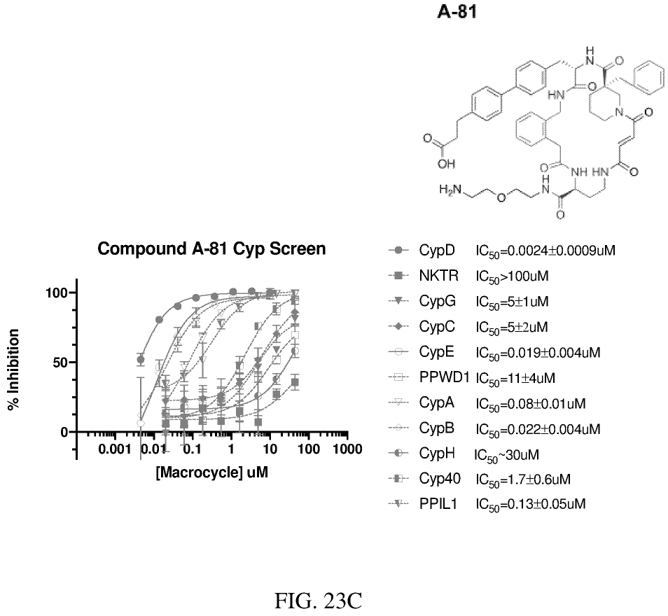

There are unique structural features of A81-binding. There are small S2 pocket ligands (see FIG. 22A) and large S2 pocket ligands (see FIG. 22B). There is a loop that contains gatekeepers and flips out depending on the ligand present. There is also a side chain of gatekeepers that drastically changes position based on the ligand (see FIGS. 22A-22B).

Phenotypic Analysis of CypD Inhibitors in Cell Culture and Isolated Mitochondria Inhibition of mPtP in Isolated Mitochondria

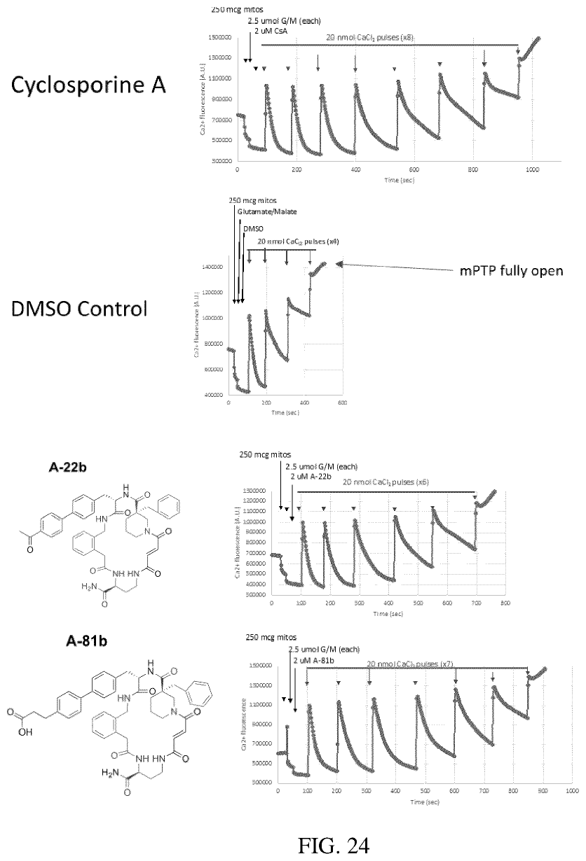

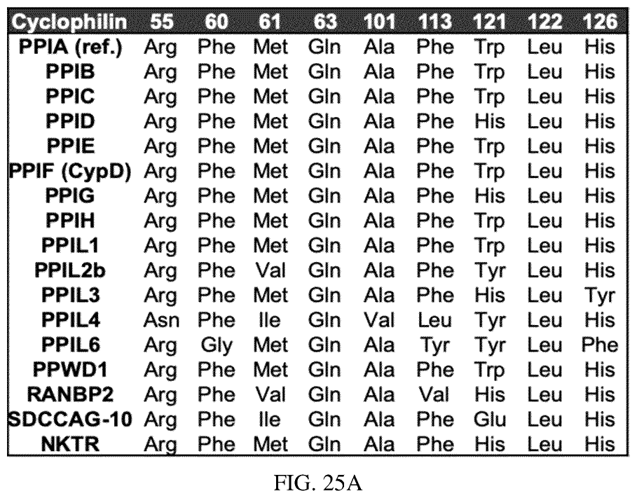

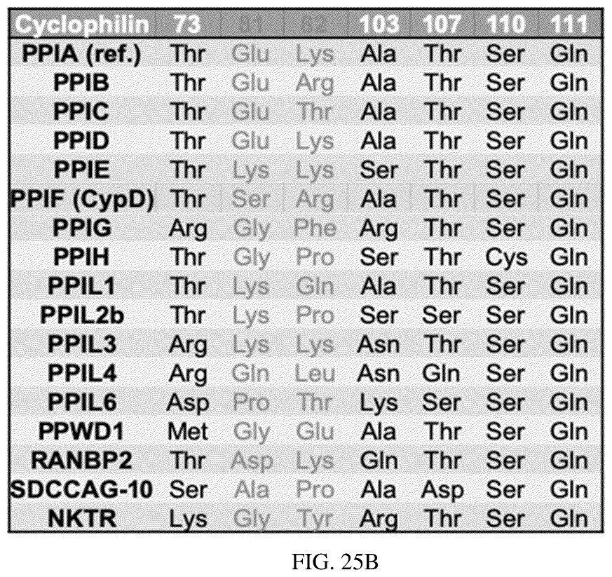

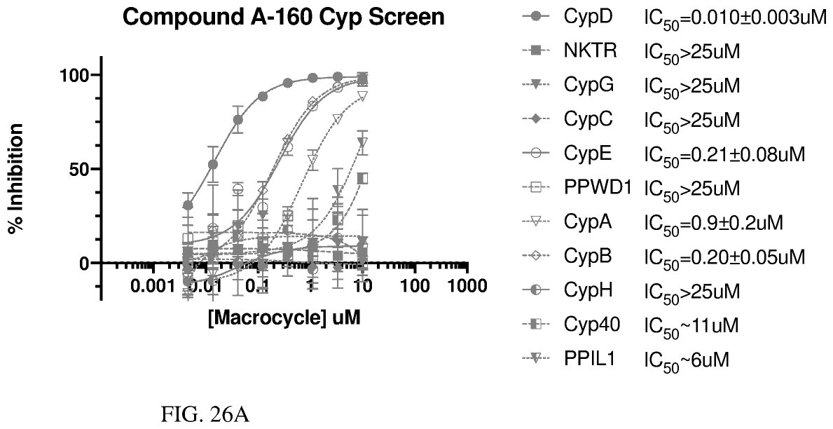

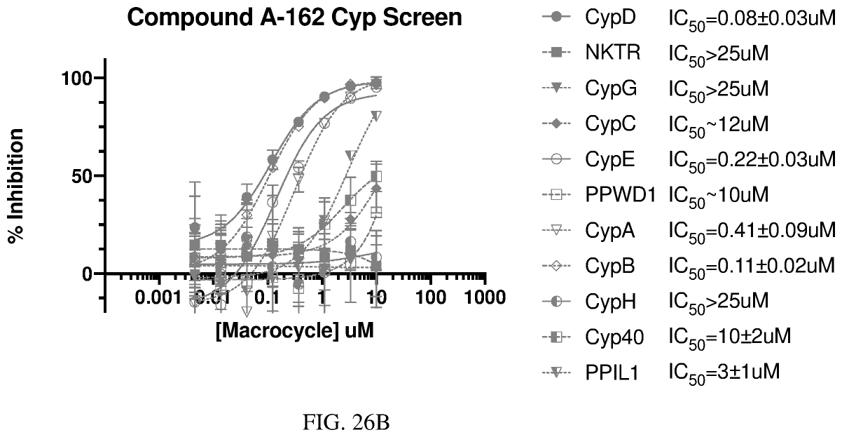

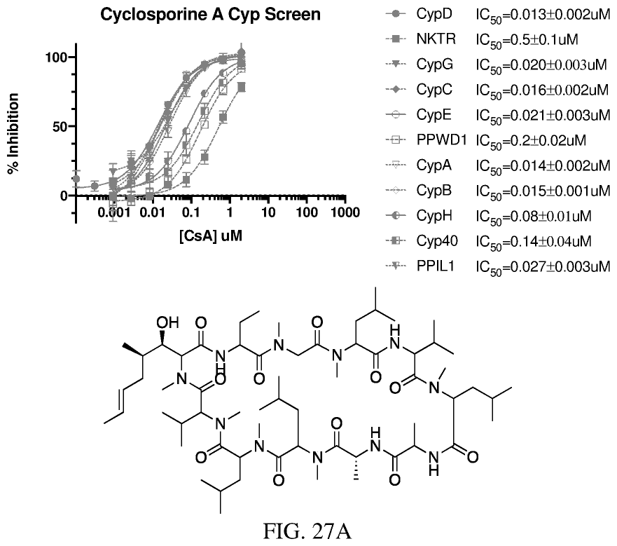

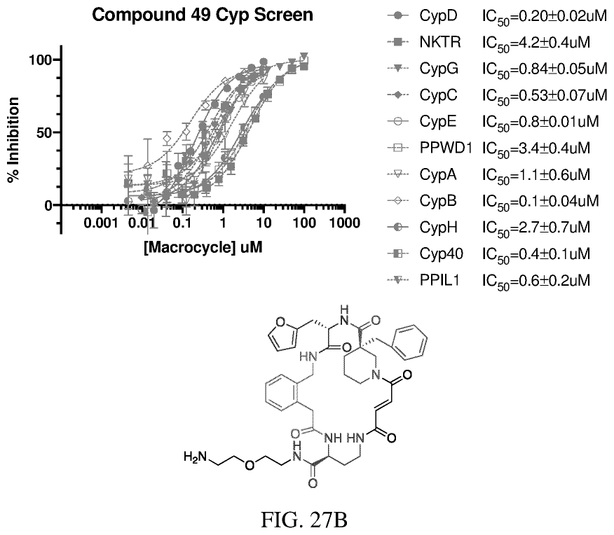

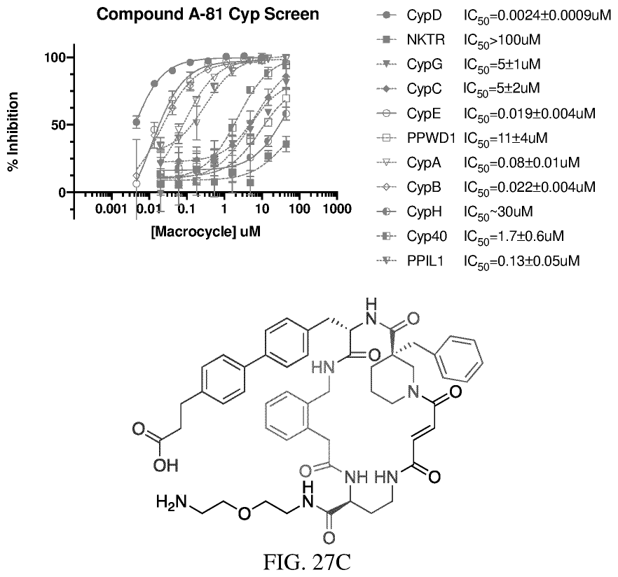

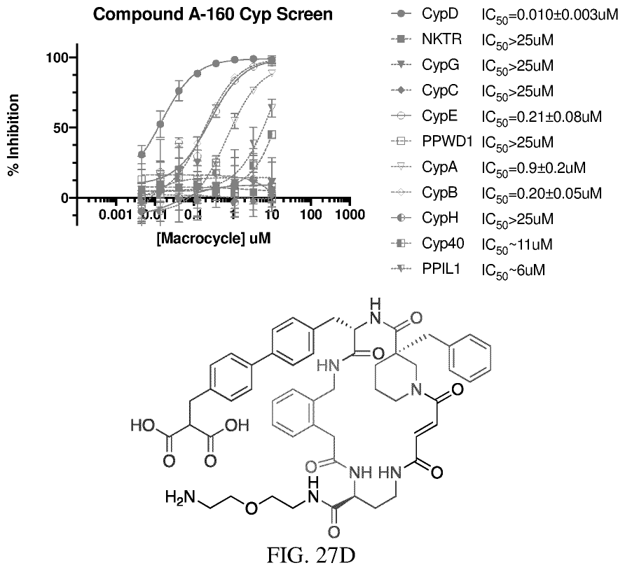

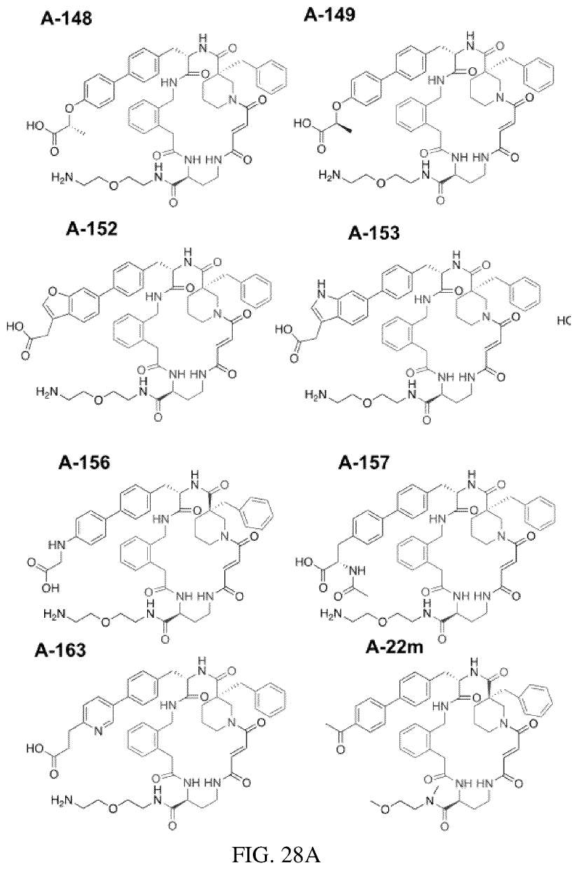

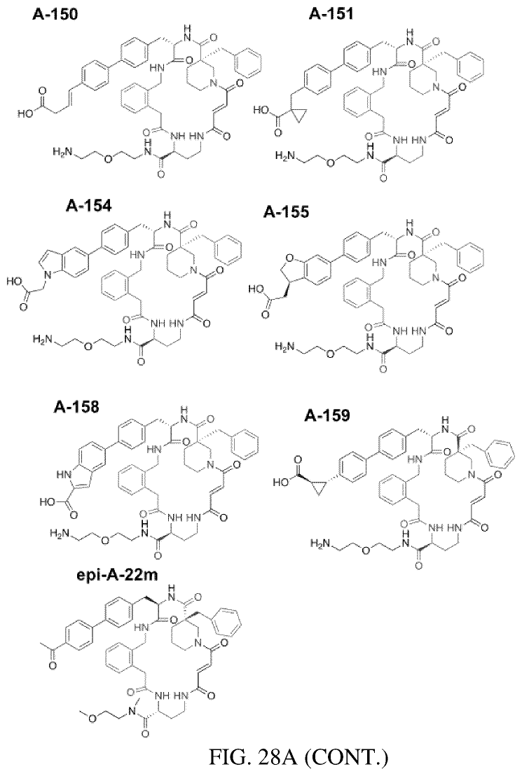

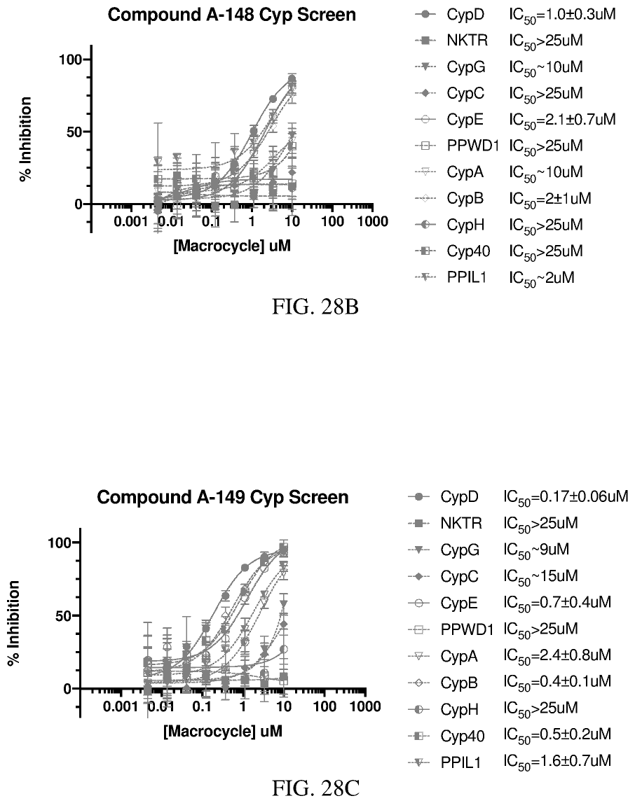

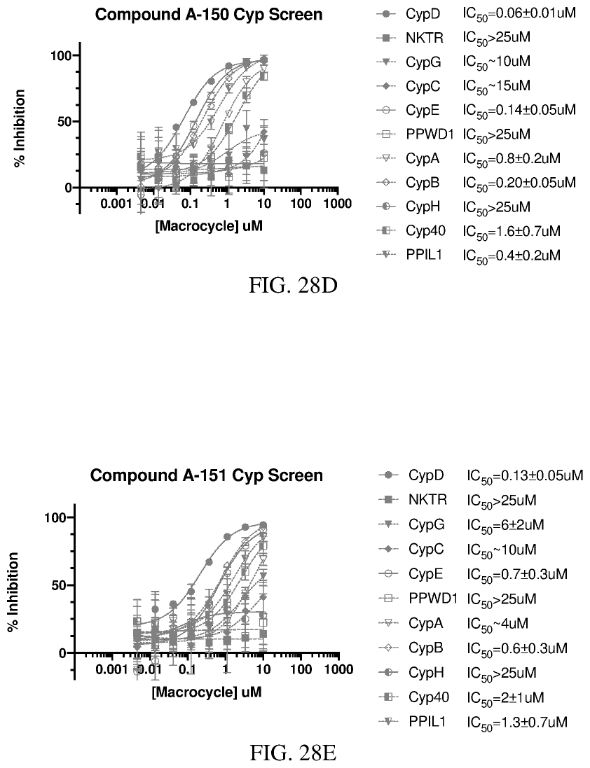

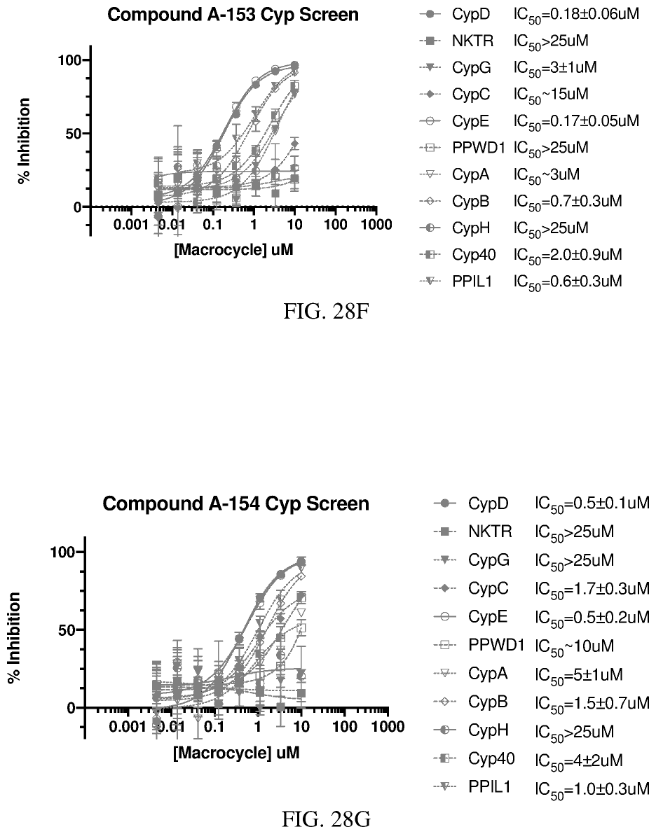

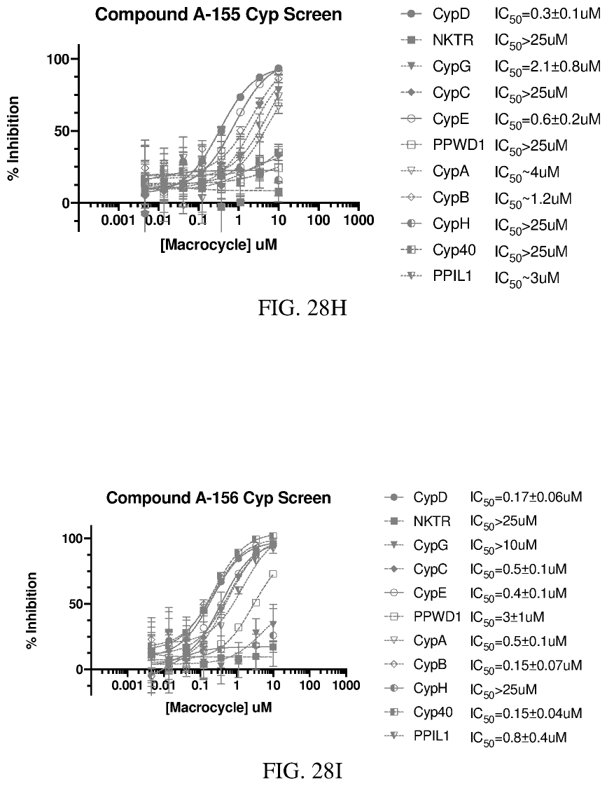

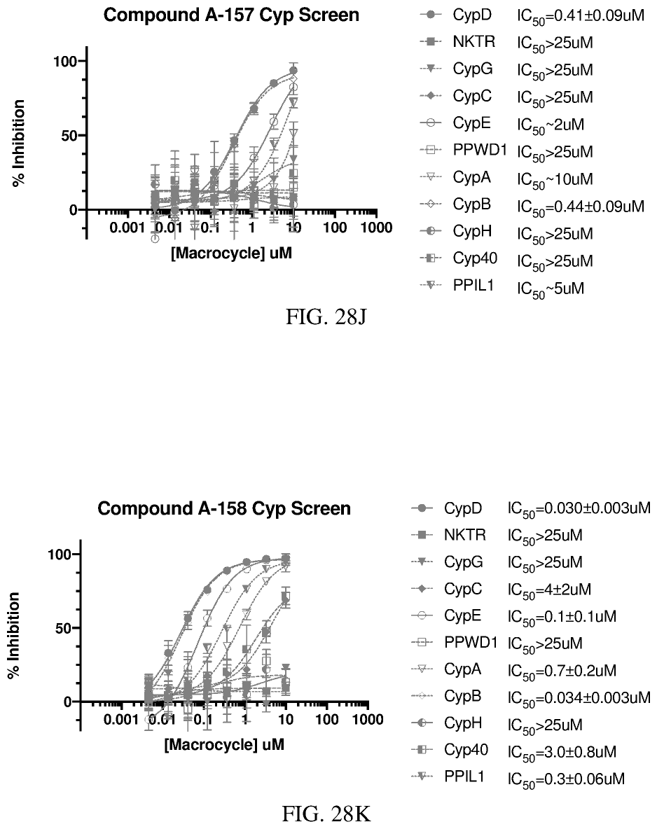

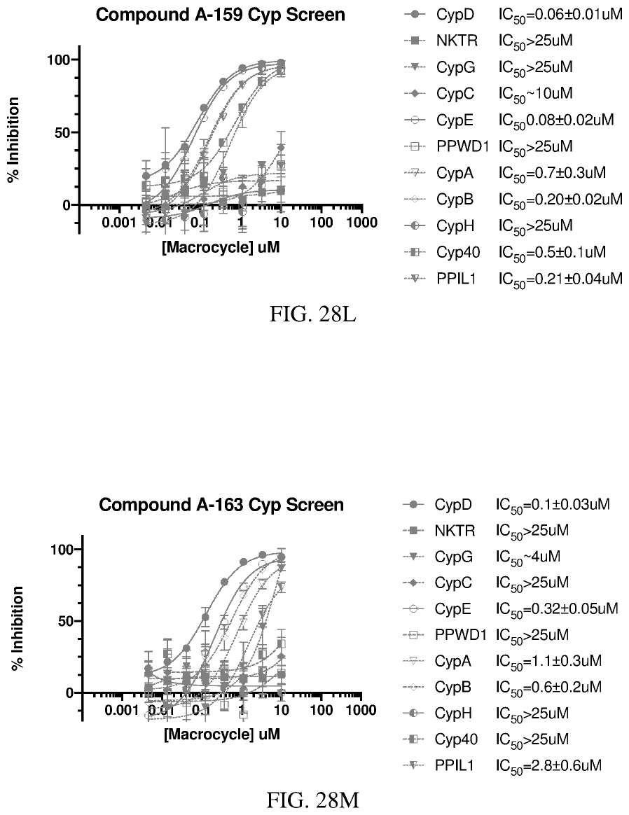

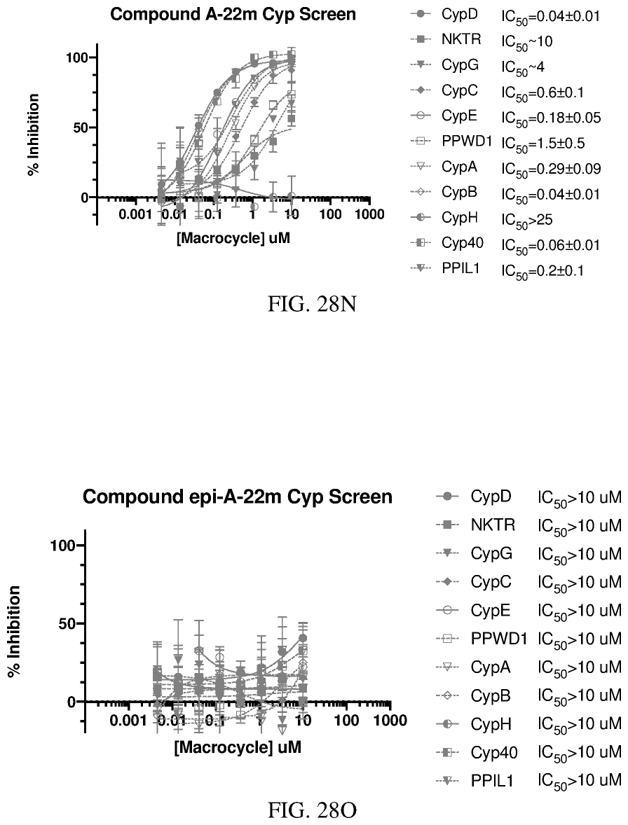

In isolated mice kidney mitochondria, the maximum number of Ca²⁺ pulses that could be tolerated before full mPTP opening was measured (see FIG. 24). Administration of exemplary CypD inhibitors extended the number of tolerated pulses. CsA, a positive control CypD inhibitor doubled the pulse number. A-22b and A-81b extended pulse number, with A-81b almost as potent as CsA. The in vitro inhibition data translates to phenotypic CypD inhibition in isolated mitochondria (see FIG. 24).

Inhibition of mPtP in In Vitro Cell Culture Using Exemplary CypD Inhibitors

As disclosed herein, macrocycle inhibitors of cyclophilin D have been discovered and designed with previously unparalleled specificity. An active site binder displaying weak inhibition (IC₅₀=15 uM) and no selectivity profile was initially optimized to 200 nM. CypD specificity was designed by building into S2 pocket residues. Bi-phenyl para-carboxylates provided the greatest level of potency (<2-30 nM) and selectivity for CypD. Co-crystal structures of these macrocycles bound to CypD were solved, and a dual binding mode was determined between active site and S2 pocket. MD simulations of co-crystal structures were conducted to observe dynamic interactions of carboxylate with S2 pocket. Initial proof of target engagement was determined via inhibition of the mPTP. Exemplary compounds A-81 and A-22 can extend Ca²⁺ loading in isolated mitochondria before mPTP opening occurs. Compound A-81 appears to not rescue mPTP in cell culture. Alternate macrocycle A-22 shows minimal rescue and can be optimized for cell permeability by modification of its tail region.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I′): 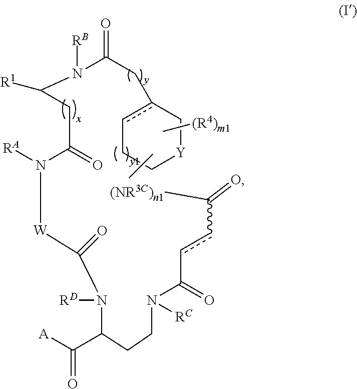
or a pharmaceutically acceptable salt, solvate, hydrate, stereoisomer, polymorph, tautomer, isotopically enriched form, or prodrug thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
A is —OR^{5A} or —N(R⁵)₂;
W is an optionally substituted C₁₋₆ hydrocarbon chain, optionally wherein one or more carbon units of the hydrocarbon chain are independently replaced with substituted or unsubstituted phenylene, substituted or unsubstituted carbocyclylene, —O—, or —N(R^{W})—;
R¹ is substituted or unsubstituted C₁₋₆ alkyl, substituted or unsubstituted alkenyl, substituted or unsubstituted carbocyclyl, or substituted or unsubstituted aryl;
X is —O—, —C(R^{3A})₂—, —C(R^{3A})═, or —N(R^{3B})—, as valency permits;
Y is —C(R^{3A})₂— or —N(R^{3B})—, as valency permits;
each instance of R^{3A} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or optionally wherein two instances of R^{3A} are joined together with the intervening atoms to form an optionally substituted heterocyclyl or heteroaryl ring;
R^{c1} is halogen, hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, a sulfur protecting group when attached to a sulfur atom, —NH₂, —N(optionally substituted alkyl)₂, —OH, or —O(optionally substituted alkyl);
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{3B} is hydrogen, substituted or unsubstituted acyl, substituted or unsubstituted alkyl, or a nitrogen protecting group;
R^{3C} is hydrogen, substituted or unsubstituted acyl, substituted or unsubstituted alkyl, or a nitrogen protecting group;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN;
each instance of R⁵ is independently hydrogen, substituted or unsubstituted acyl, substituted or unsubstituted alkyl, substituted or unsubstituted alkenyl, substituted or unsubstituted alkynyl, or a nitrogen protecting group;
R^{5A} is hydrogen, substituted or unsubstituted acyl, substituted or unsubstituted alkyl, substituted or unsubstituted alkenyl, substituted or unsubstituted alkynyl, or an oxygen protecting group;
each of R^{A}, R^{B}, R^{C}, R^{D}, and R^{W} is independently hydrogen, substituted or unsubstituted alkyl, substituted or unsubstituted acyl, or a nitrogen protecting group;
x is 0 or 1;
y is 0 or 1;
y1 is 0 or 1;
m1 is 0, 1, 2, 3, 4, 5, or 6; and
n1 is 0 or 1;
provided that the compound is not of formula:

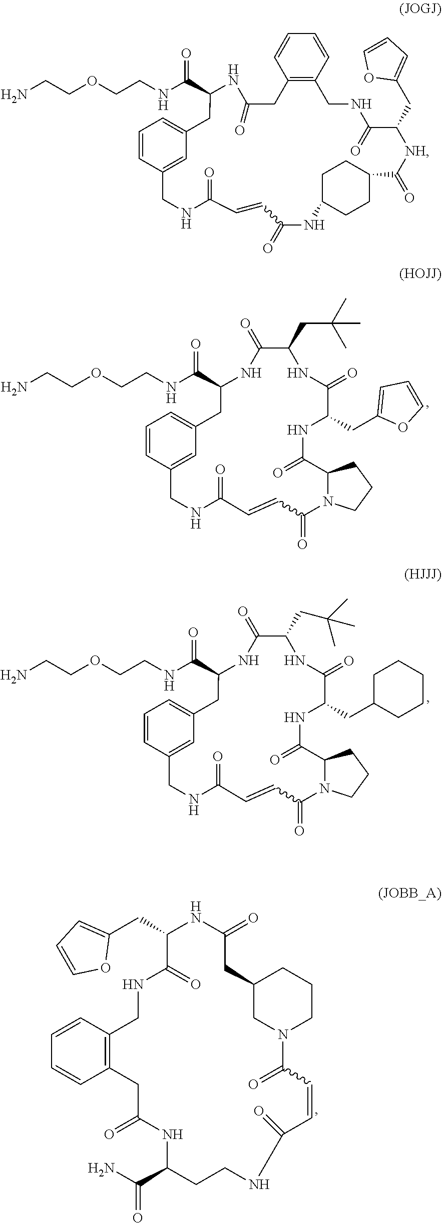 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

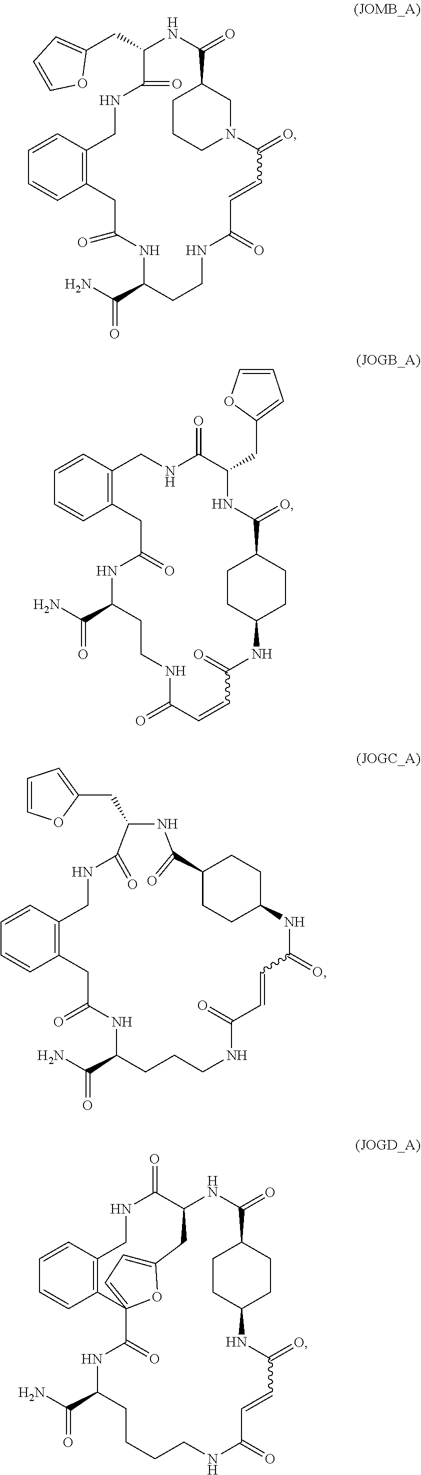

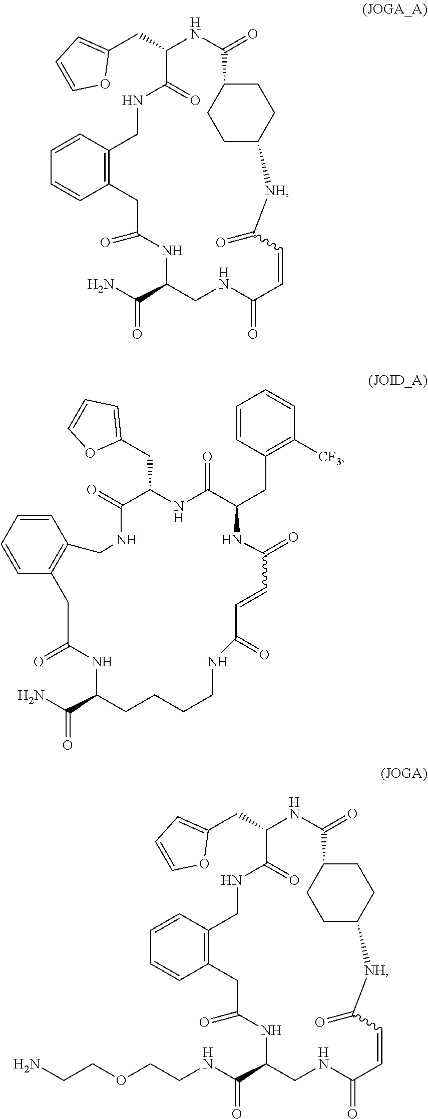 [SMILES: NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1 ; NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1]

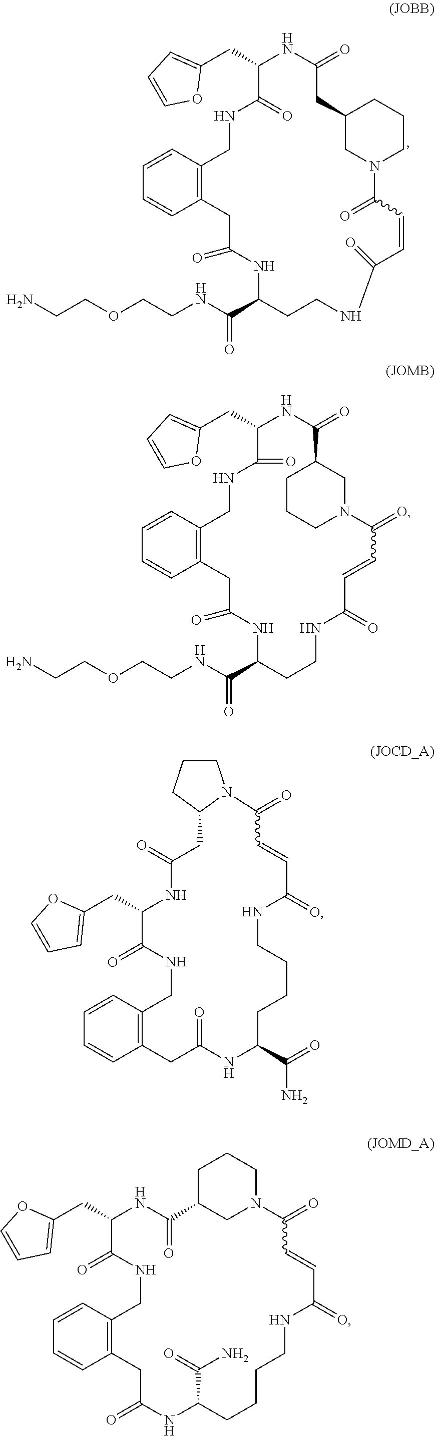

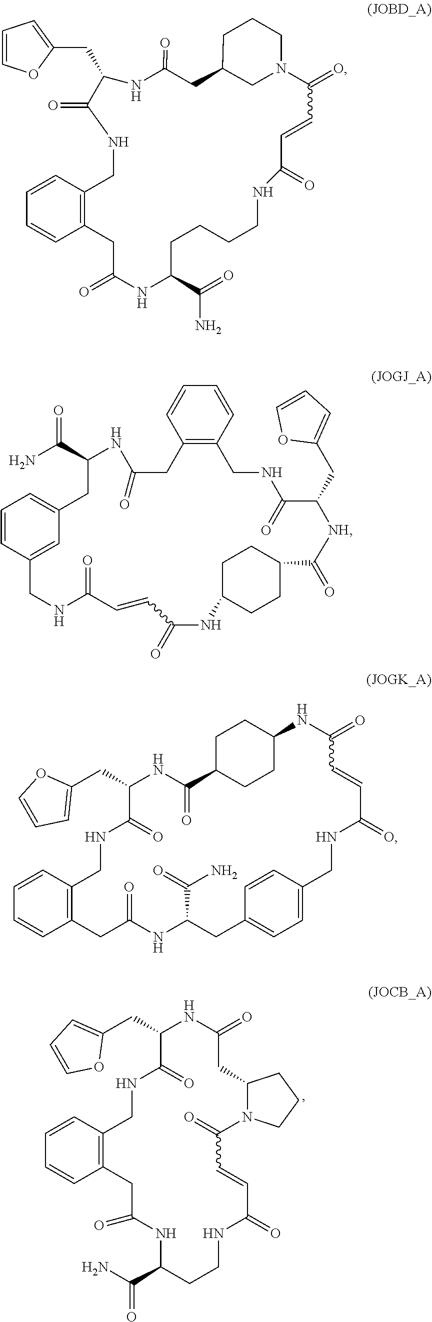 [SMILES: NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1]

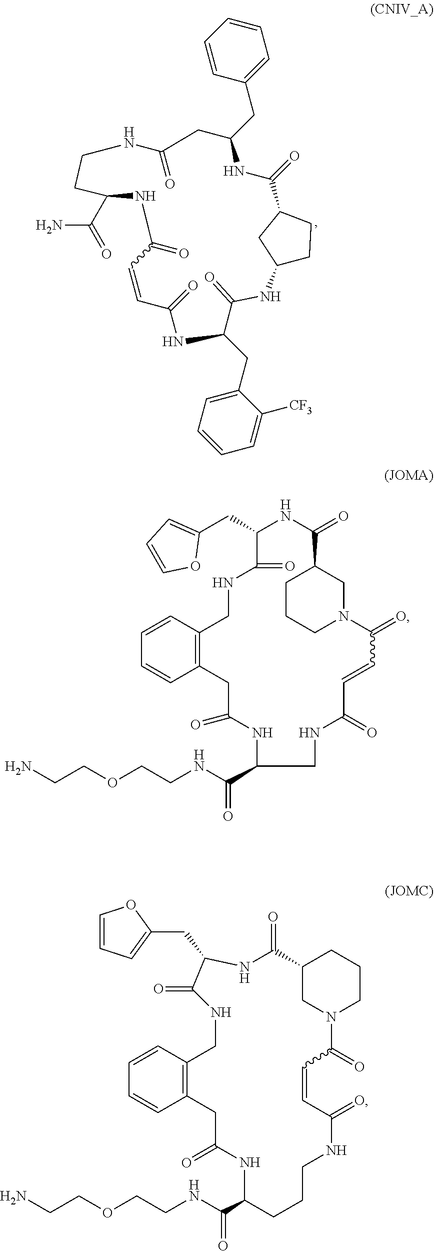 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1]

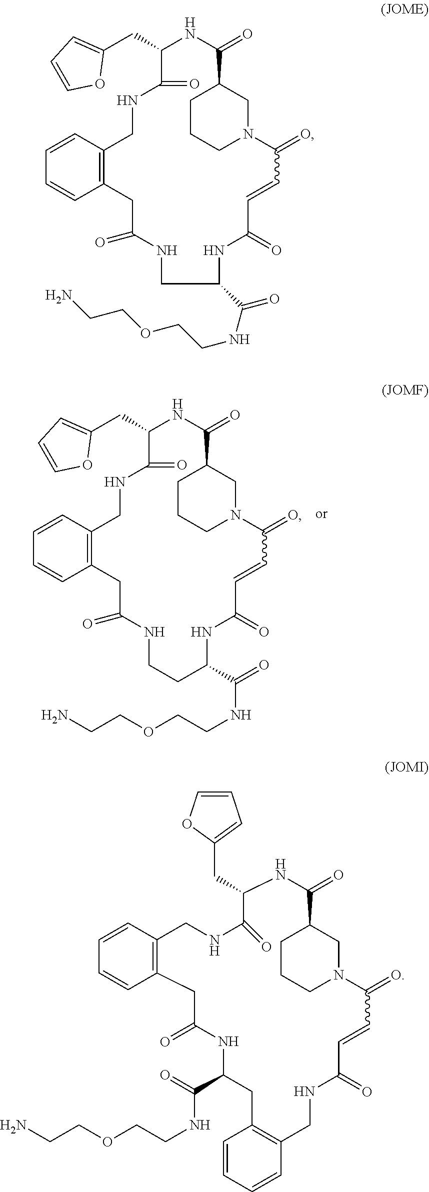 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

2. The compound of claim 1, wherein the compound is of Formula (I): 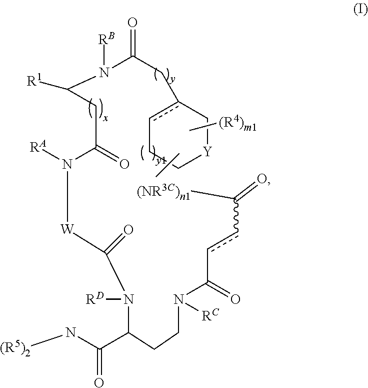
or a pharmaceutically acceptable salt, solvate, hydrate, stereoisomer, polymorph, tautomer, isotopically enriched form, or prodrug thereof, wherein:
each instance of [symbol] is independently a single or double C—C bond, as valency permits, wherein when [symbol] is a double C—C bond adjacent to [symbol] , then [symbol] indicates that the adjacent C—C double bond may be in a cis or trans configuration;
W is optionally substituted C₁₋₆ hydrocarbon chain, optionally wherein one or more carbon units of the hydrocarbon chain are independently replaced with substituted or unsubstituted phenylene, or substituted or unsubstituted carbocyclylene, —O—, or —N(R^{W})—;
R¹ is substituted or unsubstituted C₁₋₆ alkyl, substituted or unsubstituted alkenyl, substituted or unsubstituted carbocyclyl, or substituted or unsubstituted aryl;
X is —O—, —C(R^{3A})₂—, —C(R^{3A})═, or —N(R^{3B})—, as valency permits;
Y is —C(R^{3A})₂— or —N(R^{3B})—, as valency permits;
each instance of R^{3A} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or optionally wherein two instances of R^{3A} are joined together with the intervening atoms to form an optionally substituted heterocyclyl or heteroaryl ring;
R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
R^{3B} is hydrogen, substituted or unsubstituted acyl, substituted or unsubstituted alkyl, or a nitrogen protecting group;
R^{3C} is hydrogen, substituted or unsubstituted acyl, substituted or unsubstituted alkyl, or a nitrogen protecting group;
R⁴ is halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN;
each instance of R⁵ is independently hydrogen, substituted or unsubstituted acyl, substituted or unsubstituted alkyl, substituted or unsubstituted alkenyl, substituted or unsubstituted alkynyl, or a nitrogen protecting group;
each of R^{A}, R^{B}, R^{C}, R^{D}, and R^{W} is independently hydrogen, substituted or unsubstituted alkyl, substituted or unsubstituted acyl, or a nitrogen protecting group;
x is 0 or 1;
y is 0 or 1;
y1 is 0 or 1;
m1 is 0, 1, 2, 3, 4, 5, or 6; and
n1 is 0 or 1;
provided that the compound is not of formula:

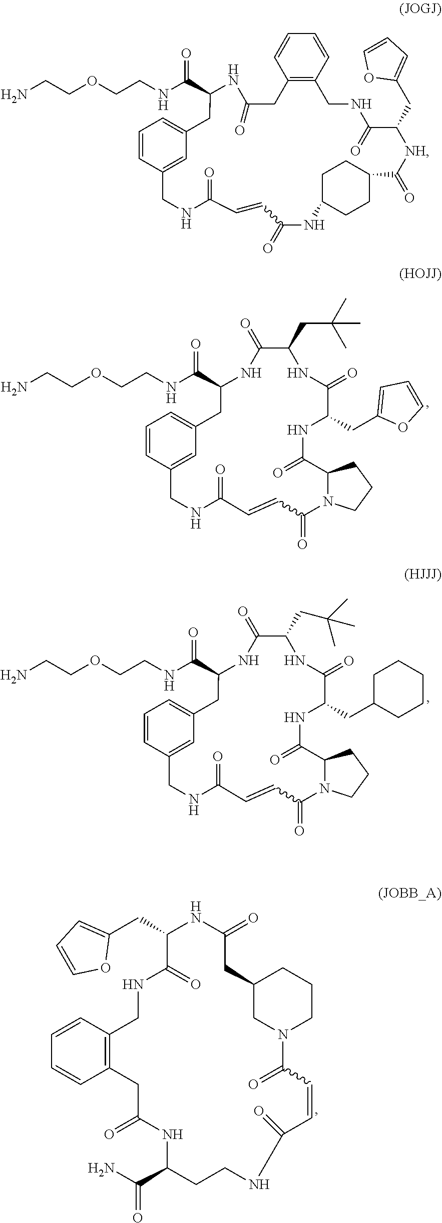 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](Cc2ccco2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2]

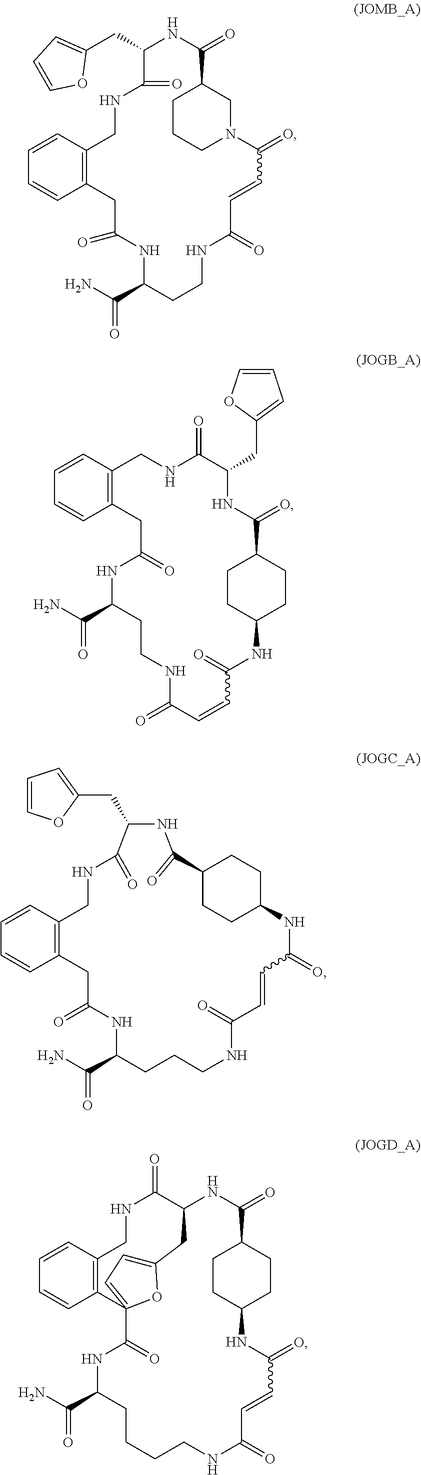

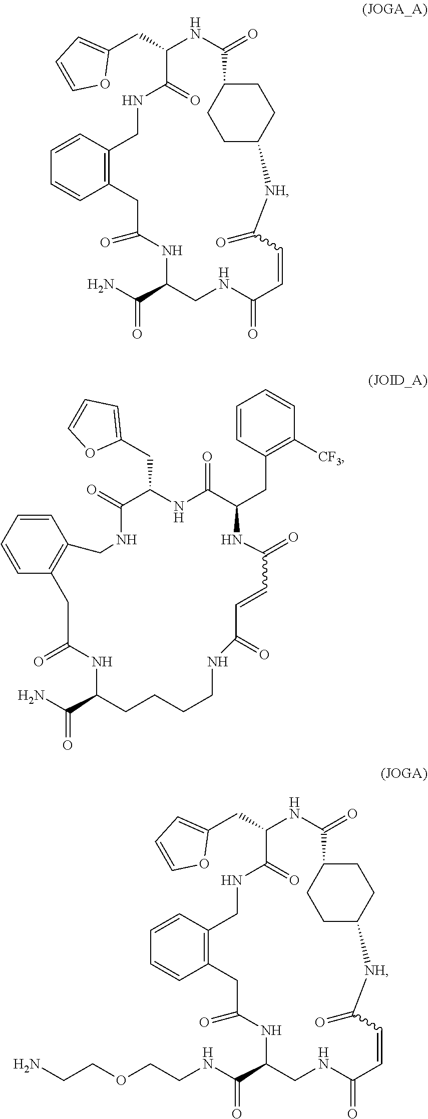 [SMILES: NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1 ; NCCOCCNC(=O)[C@@H]1CNC(=O)C=CC(=O)N[C@H]2CC[C@H](CC2)C(=O)N[C@@H](Cc2ccco2)C(=O)NCc2ccccc2CC(=O)N1]

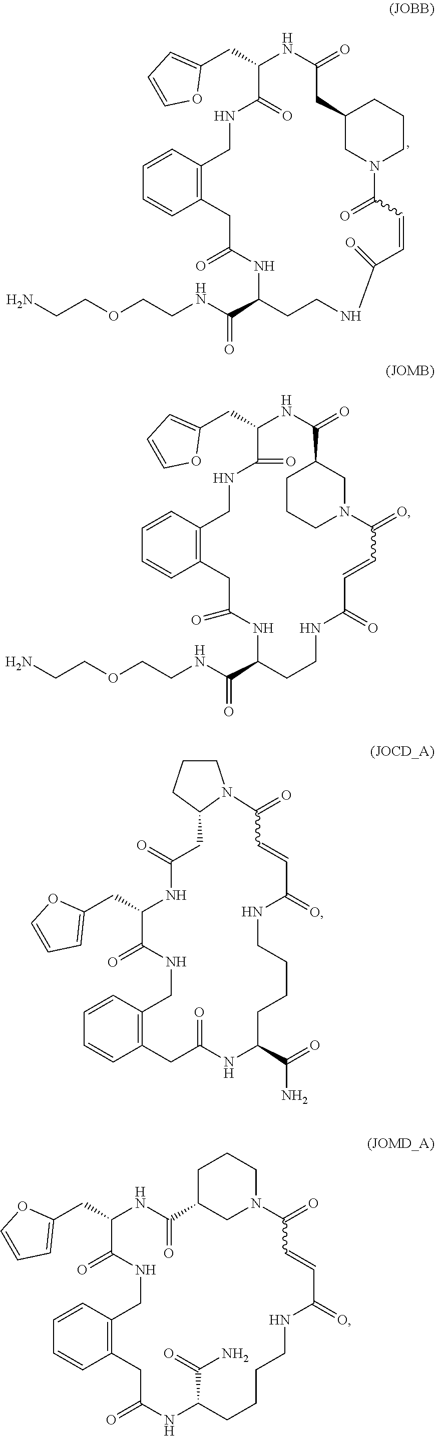

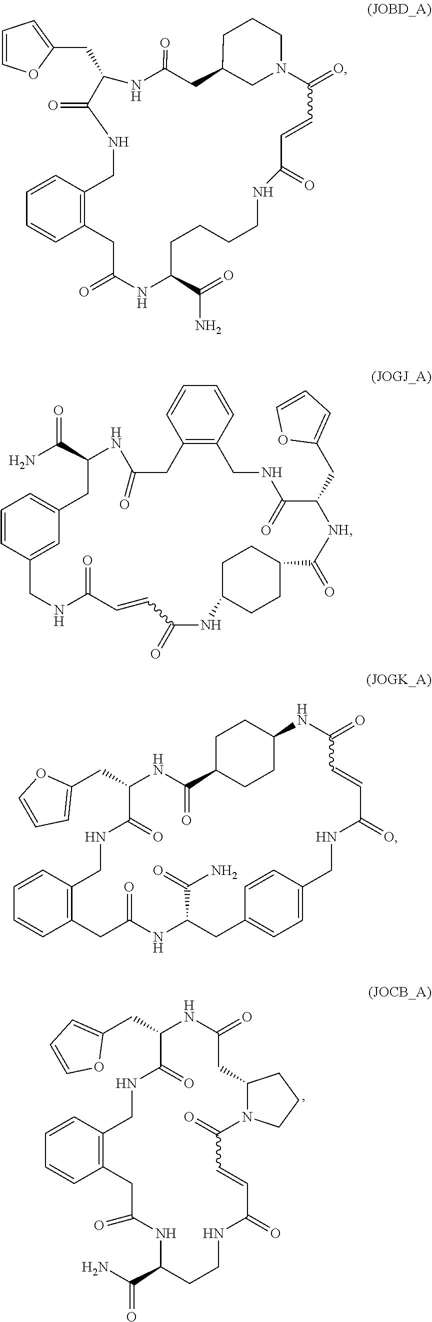 [SMILES: NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCNC(=O)C=CC(=O)N2CCC[C@H]2CC(=O)N[C@@H](Cc2ccco2)C(=O)NCc2ccccc2CC(=O)N1]

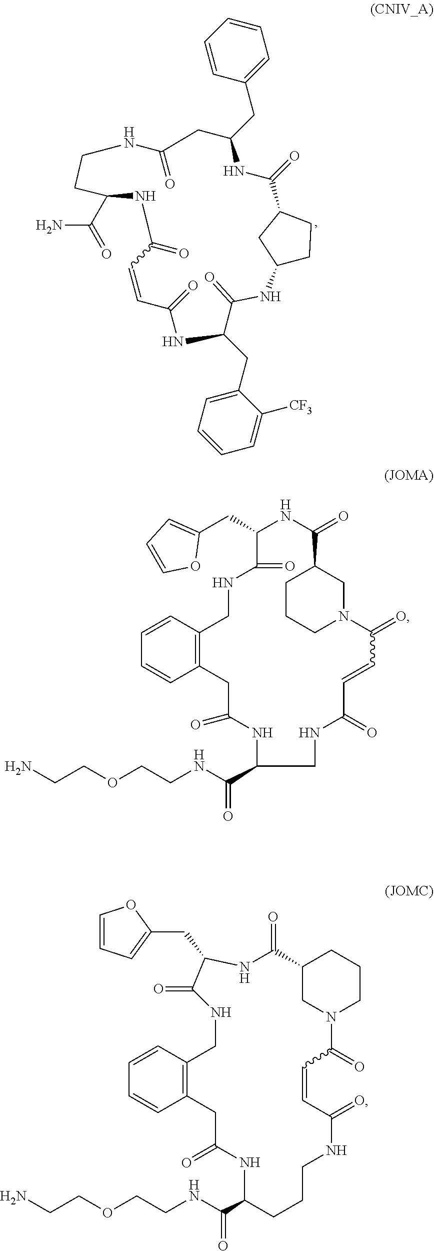 [SMILES: NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)C=CC(=O)N1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1]

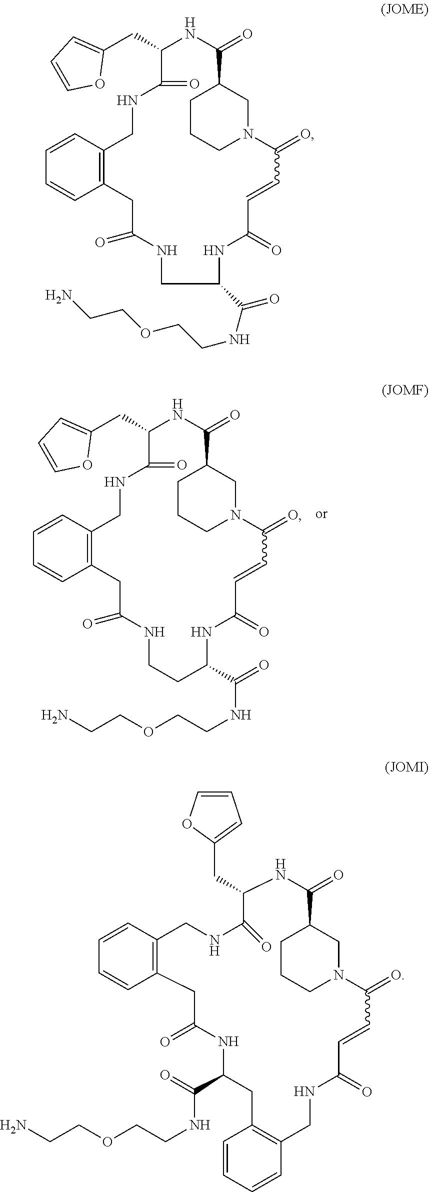 [SMILES: NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1 ; "or" ; NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]

3. The compound of claim 1 or 2, wherein the compound of Formula (I′) is of formula: 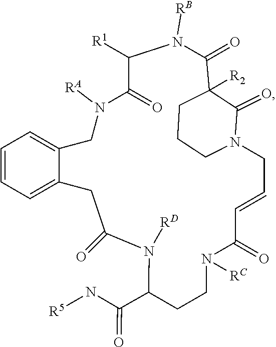
or a pharmaceutically acceptable salt, solvate, hydrate, stereoisomer, polymorph, tautomer, isotopically enriched form, or prodrug thereof, wherein:
R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN.

4. The compound of claim 1 or 2, wherein the compound of Formula (I′) is of formula: 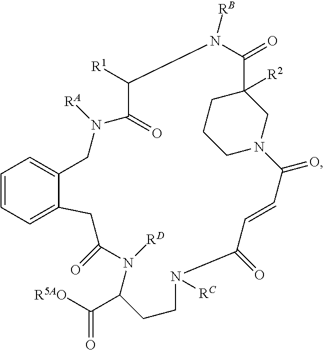
or a pharmaceutically acceptable salt, solvate, hydrate, stereoisomer, polymorph, tautomer, isotopically enriched form, or prodrug thereof, wherein:
R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN; and
R^{5A} is hydrogen or substituted or unsubstituted alkyl.

5. The compound of claim 1 or 2, wherein the compound of Formula (I′) is of formula: 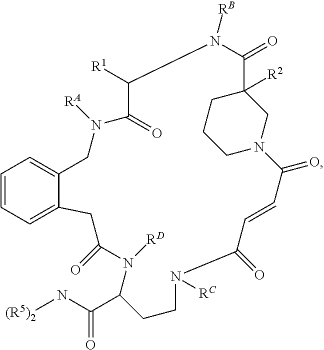
or a pharmaceutically acceptable salt, solvate, hydrate, stereoisomer, polymorph, tautomer, isotopically enriched form, or prodrug thereof, wherein:
R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN.

6. The compound of any one of claims 1-5, wherein the compound of Formula (I′) is of formula: 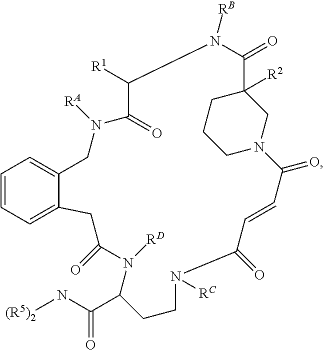
or a pharmaceutically acceptable salt thereof, wherein:
R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN.

7. The compound of any one of claims 1-5, wherein the compound of Formula (I′) is of formula: 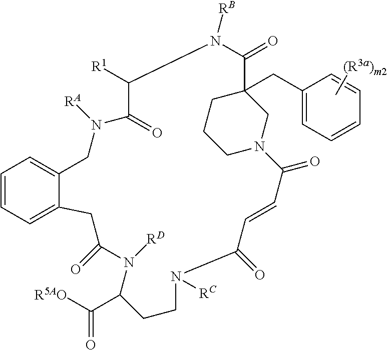
or a pharmaceutically acceptable salt thereof, wherein:
R² is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or —CN; and
R^{5A} is hydrogen or substituted or unsubstituted alkyl.

8. The compound of any one of claims 1-7, wherein the compound of Formula (I′) is of formula: 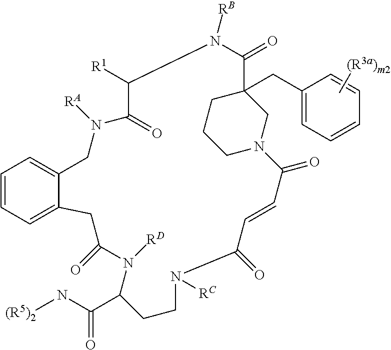
or a pharmaceutically acceptable salt, solvate, hydrate, stereoisomer, polymorph, tautomer, isotopically enriched form, or prodrug thereof, wherein:
each instance of R^{3a} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂,
—N(R^{c2})₂, —SR^{c1}, —CN, or —SCN; and
m2 is 0, 1, 2, 3, 4, or 5.

9. The compound of any one of claims 1-8, wherein the compound is of formula: 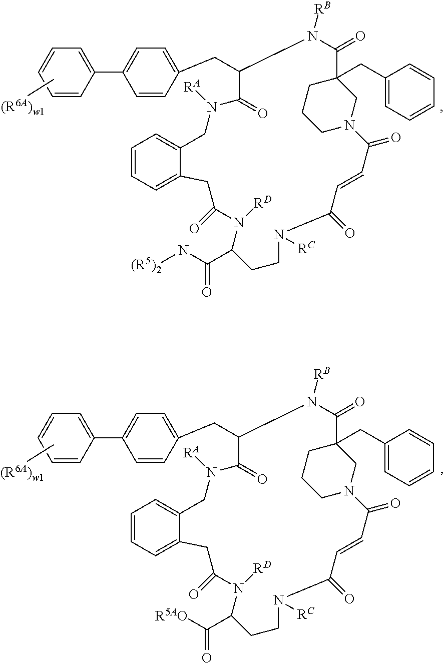
or a pharmaceutically acceptable salt, solvate, hydrate, stereoisomer, polymorph, tautomer, isotopically enriched form, or prodrug thereof, wherein:
each instance of R^{6A} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, —B(OR^{6c1})₂, or —SCN; and
w1 is 0, 1, 2, 3, 4, 5, or 6.

10. The compound of any one of claims 1-9, wherein the compound is of formula: 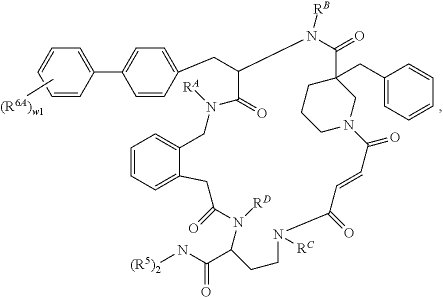
or a pharmaceutically acceptable salt, solvate, hydrate, stereoisomer, polymorph, tautomer, isotopically enriched form, or prodrug thereof, wherein:
each instance of R^{6A} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, —B(OH)₂, or —SCN; and
w1 is 0, 1, 2, 3, 4, 5, or 6.

11. The compound of any one of claims 1-10, wherein the compound is of formula: 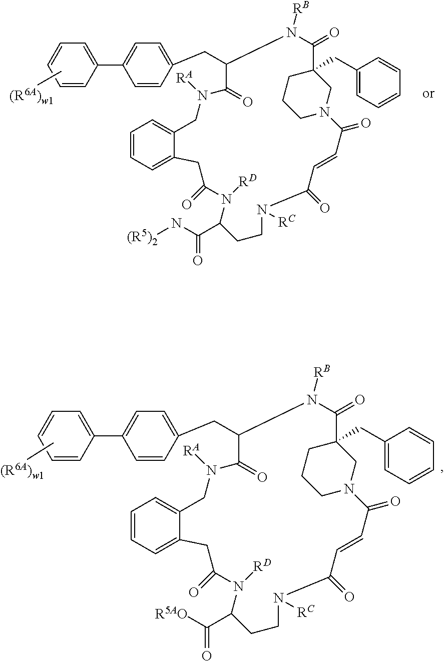
or a pharmaceutically acceptable salt, solvate, hydrate, stereoisomer, polymorph, tautomer, isotopically enriched form, or prodrug thereof.

12. The compound of any one of claims 1-11, wherein the compound is of formula: 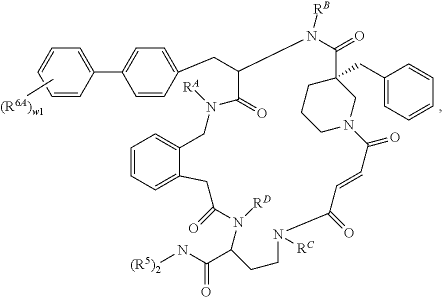
or a pharmaceutically acceptable salt, solvate, hydrate, stereoisomer, polymorph, tautomer, isotopically enriched form, or prodrug thereof.

13. The compound of any one of claims 1-11, wherein the compound is of formula: 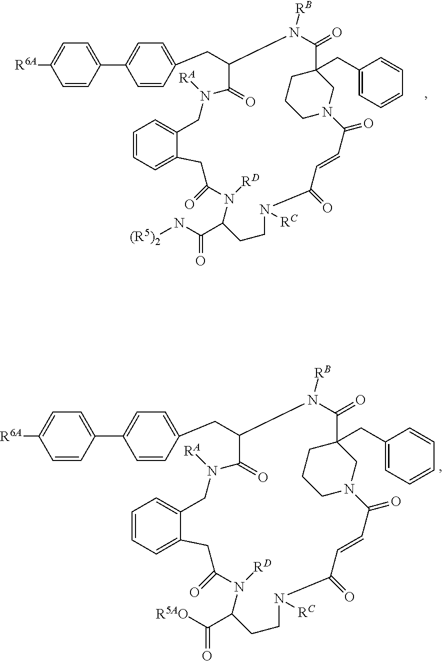
or a pharmaceutically acceptable salt, solvate, hydrate, stereoisomer, polymorph, tautomer, isotopically enriched form, or prodrug thereof, wherein:
R^{6A} is substituted or unsubstituted acyl, —C(═O)N(R^{6c})₂, 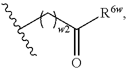
—NO₂, —SO₂R^{c1}, —SO₂N(R^{6c})₂, —B(OR^{6c1})₂, or —OR^{c1};
each instance of R^{6c} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group;
R^{6d} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or an oxygen protecting group; and
R^{6w} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group;
w2 is 0, 1, 2, 3, 4, 5, or 6.

14. The compound of any one of claims 1-11 or 13, wherein the compound is of formula: 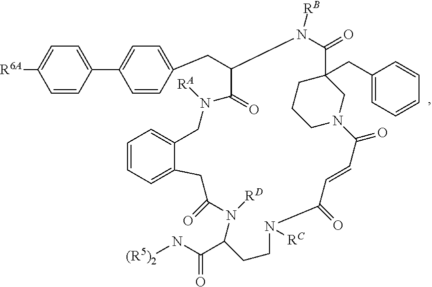
or a pharmaceutically acceptable salt, solvate, hydrate, stereoisomer, polymorph, tautomer, isotopically enriched form, or prodrug thereof, wherein:
R^{6A} is substituted or unsubstituted acyl, —C(═O)N(R^{6c})₂, 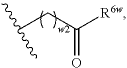
—NO₂, —SO₂R^{c1}, —SO₂N(R^{6c})₂, —B(OH)₂, or —OR^{c1};
each instance of R^{6c} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group;
R^{6d} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or an oxygen protecting group; and
R^{6w} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{6d}, or a nitrogen protecting group;
w2 is 0, 1, 2, 3, 4, 5, or 6.

15. The compound of claim 1 or 2, wherein W is substituted or unsubstituted C₁₋₆ alkylene.

16. The compound of any one of claims 1, 2, or 15, wherein W is unsubstituted n-butylene.

17. The compound of claim 1 or 2, wherein W is of formula:

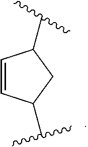 [SMILES: *C1C=CC(*)C1]

18. The compound of claim 1 or 2, wherein W is of formula:

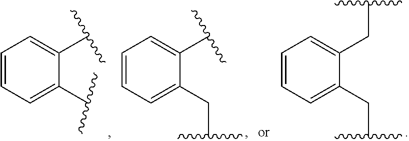

19. The compound of any one of claims 1-8, wherein R¹ is C₁₋₆ alkyl optionally substituted with substituted or unsubstituted acyl, substituted or unsubstituted alkenyl, substituted or unsubstituted carbocyclyl, substituted or unsubstituted aryl, substituted or unsubstituted heterocyclyl, or substituted or unsubstituted heteroaryl.

20. The compound of any one of claims 1-8 or 19, wherein R¹ is methyl.

21. The compound of any one of claims 1-8 or 19, wherein R¹ is 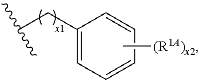
wherein:
each instance of R^{1A} is independently halogen, substituted or unsubstituted acyl, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted heteroaryl, —OR^{c1}, —NO₂, —N(R^{c2})₂, —N═C(NH₂)₂, —SR^{c1}, —SO₂, —CN, —SCN, or —OP(═O)(OH)₂, or optionally wherein two instances of R^{1A} are joined together with the intervening atoms to form an optionally substituted aryl or an optionally substituted heteroaryl group;
x1 is 0, 1, 2, 3, 4, 5, or 6;
x2 is 0, 1, 2, 3, 4, or 5.

22. The compound of any one of claims 1-8, 19, or 21, wherein R¹ is: 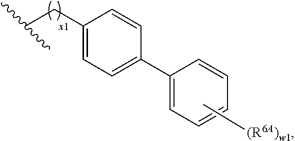
wherein:
each instance of R^{6A} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —SO₂R^{c1}, —CN, —B(OR^{6c1})₂, or —SCN;
x1 is 0, 1, 2, or 3;
w1 is 0, 1, 2, 3, 4, or 5.

23. The compound of any one of claims 9-19 or 22, wherein at least one instance of R^{6A} is 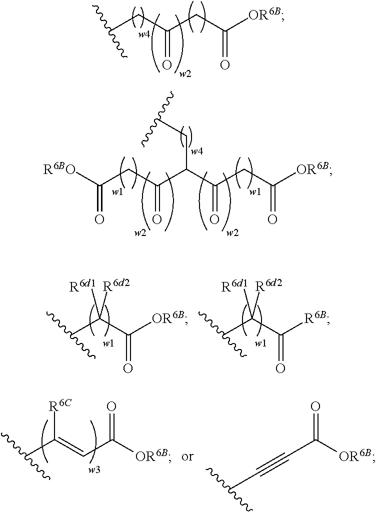
wherein:
each instance of w1 is independently 0, 1, 2, 3, 4, 5, or 6;
each instance of w2 is independently 1, 2, or 3;
w3 is 1, 2, or 3;
w4 is 0, 1, 2, or 3;
R^{6B} is hydrogen or substituted or unsubstituted alkyl;
each instance of R^{6C} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN; and
each instance of R^{6d1} and R^{6d2} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN.

24. The compound of any one of claims 9-19 or 22, wherein at least one instance of R^{6A} is 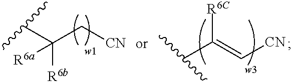
wherein:
w1 is 0, 1, 2, 3, 4, 5, or 6;
w3 is 1, 2, or 3;
R^{6a} and R^{6b} are each independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN; or optionally, R^{6a} and R^{6b} are joined together with the intervening atoms to form an optionally substituted carbocycle group; and
each instance of R^{6C} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OH, —O(alkyl), —NO₂, —NH₂, —CN, or —SCN.

25. The compound of any one of claims 1-8 or 19, wherein R¹ is 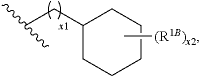
wherein:
each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN; and
x2 is 0, 1, 2, 3, 4, 5, 6, 7, 8, 9, or 10.

26. The compound of any one of claims 1-8, 19, or 21, wherein R¹ is 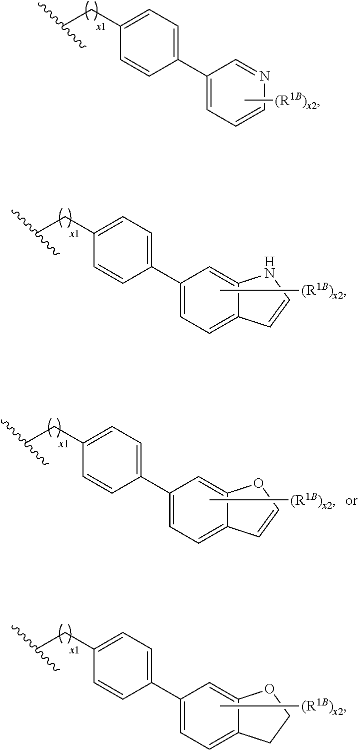
wherein:
each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, NO₂, —N(R^{c2})₂, —SR^{c1}, —CN, or —SCN, or nitrogen protecting group when attached to a nitrogen atom;
x1 is 0, 1, 2, 3, 4, 5, or 6; and
x2 is 0, 1, 2, 3, 4, 5, or 6.

27. The compound of any one of claims 1-8, 19, or 21, wherein R¹ is 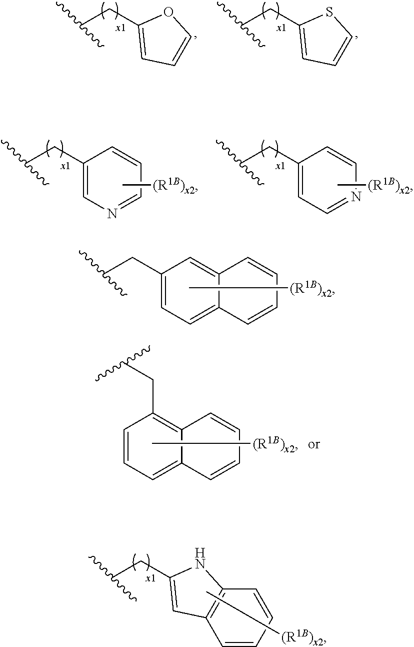
wherein:
each instance of R^{1B} is independently halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{c1}, —NO₂, —N(R^{c2})₂, —SR^{c1}, —CN, or —SCN, or nitrogen protecting group when attached to a nitrogen atom;
x1 is 0, 1, 2, 3, 4, 5, or 6; and
x2 is 0, 1, 2, 3, 4, 5, or 6.

28. The compound of any one of claims 1-8, 19, or 21-27, wherein R¹ is of formula:

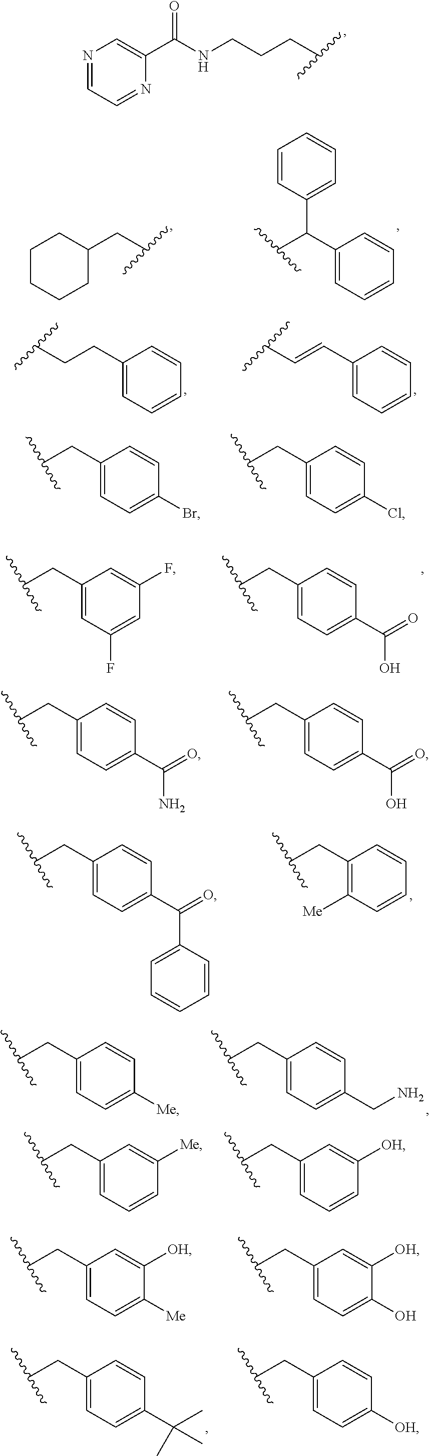

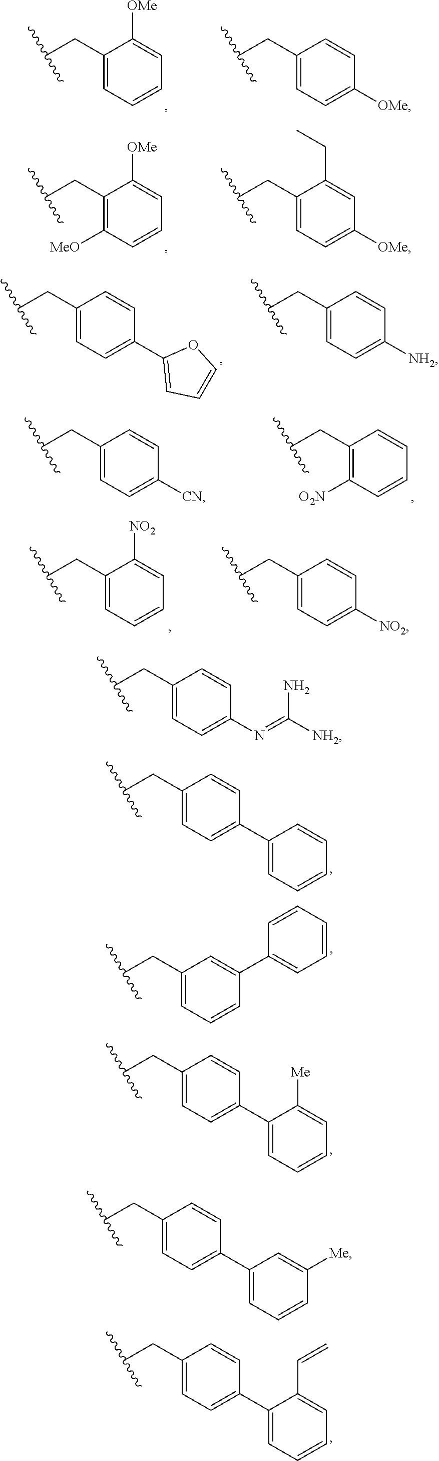

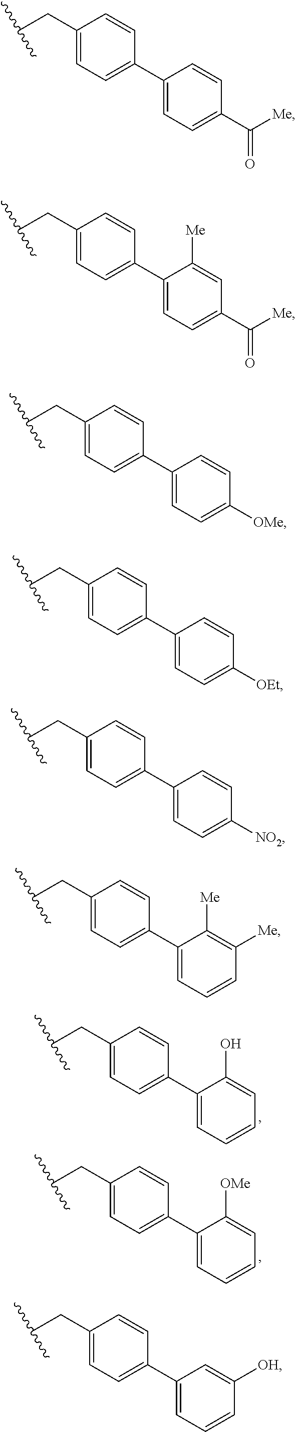

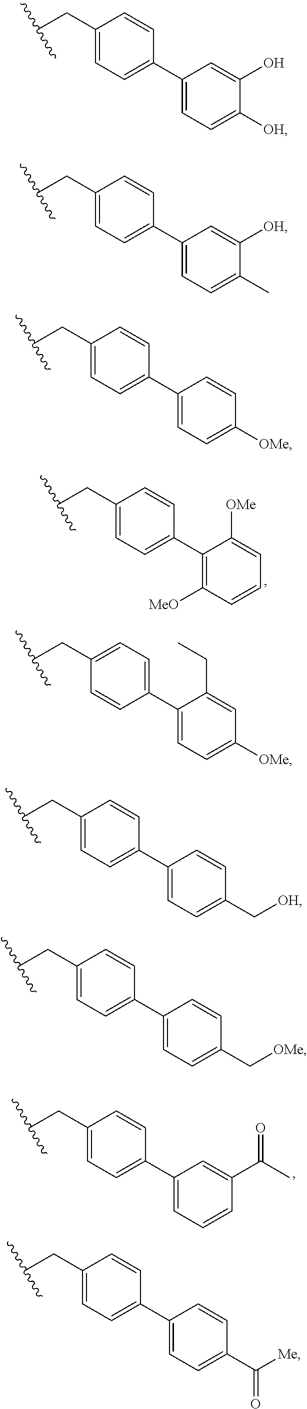

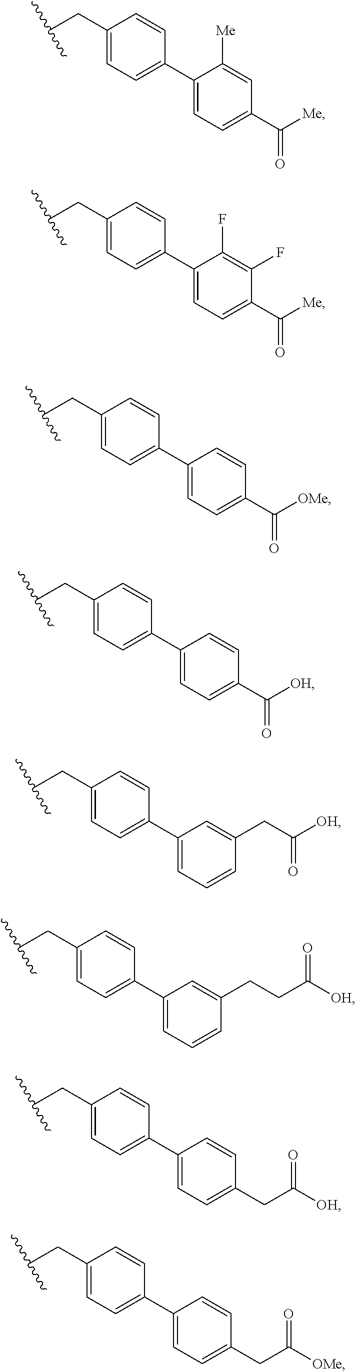

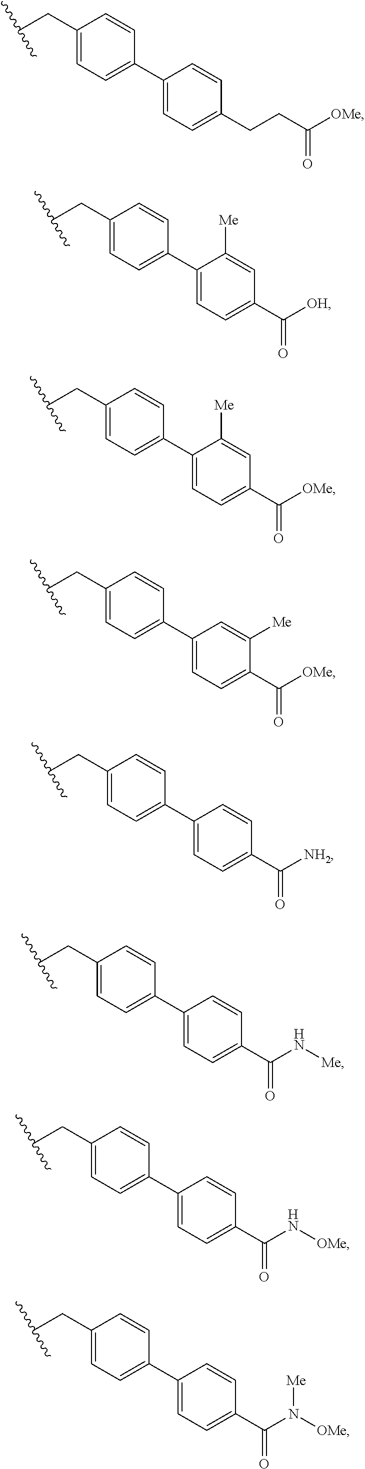

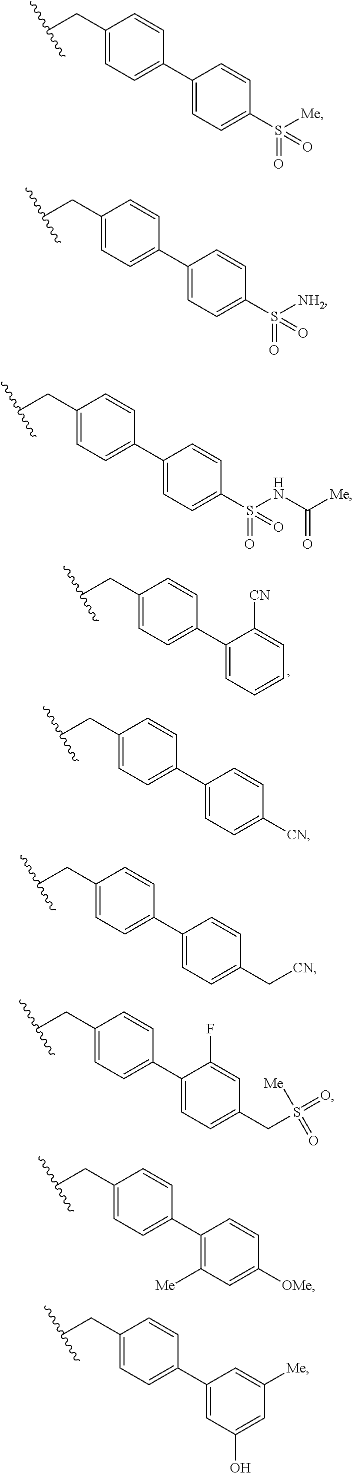

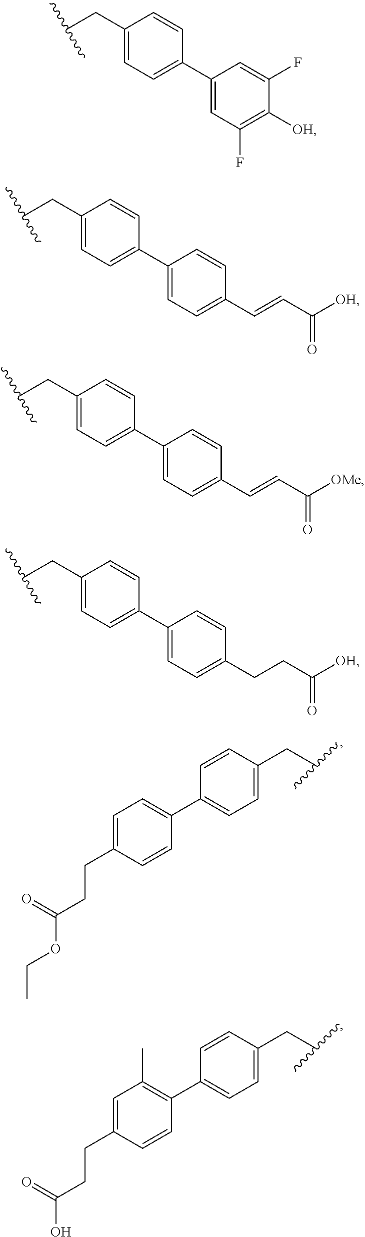

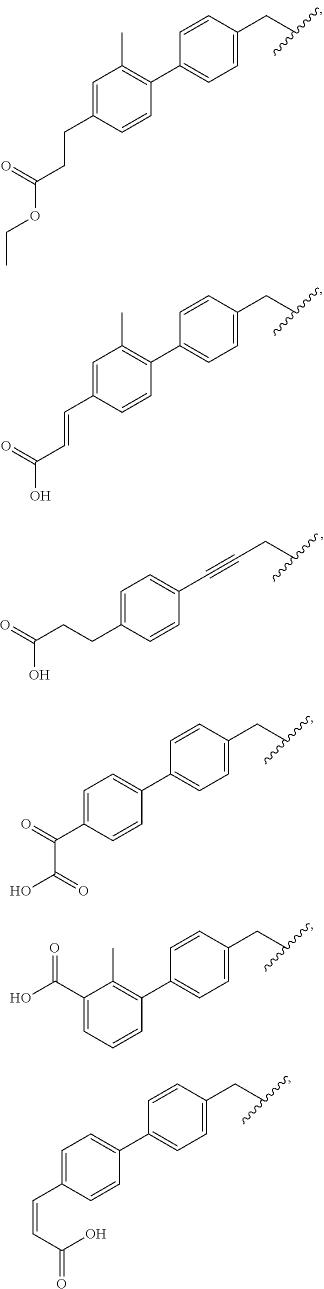 [SMILES: *Cc1ccc(-c2ccc(CCC(=O)OCC)cc2C)cc1 ; *Cc1ccc(-c2ccc(/C=C/C(=O)O)cc2C)cc1 ; *CC#Cc1ccc(CCC(=O)O)cc1 ; *Cc1ccc(-c2ccc(C(=O)C(=O)O)cc2)cc1 ; *Cc1ccc(-c2cccc(C(=O)O)c2C)cc1 ; *Cc1ccc(-c2ccc(/C=C\C(=O)O)cc2)cc1]

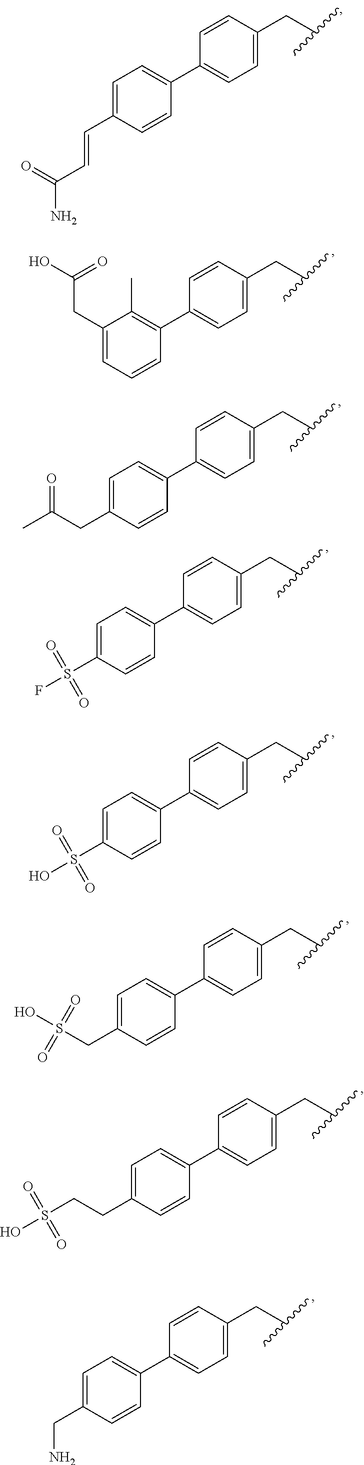

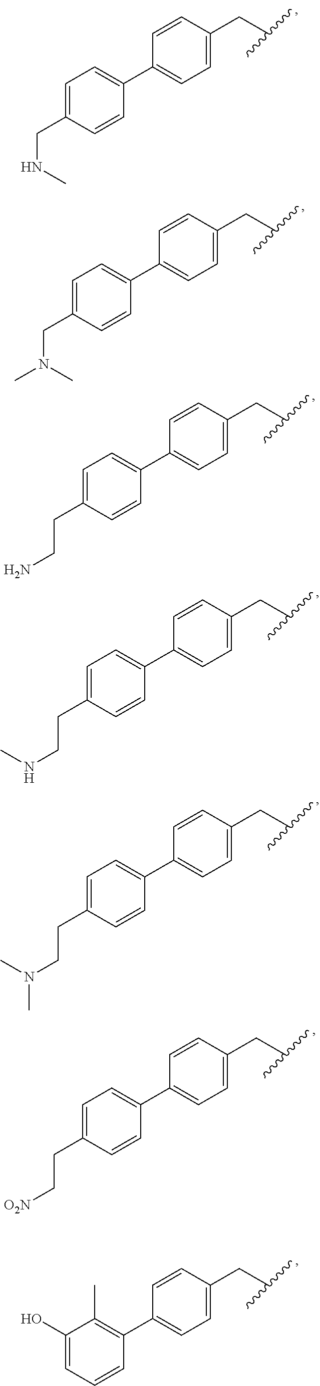

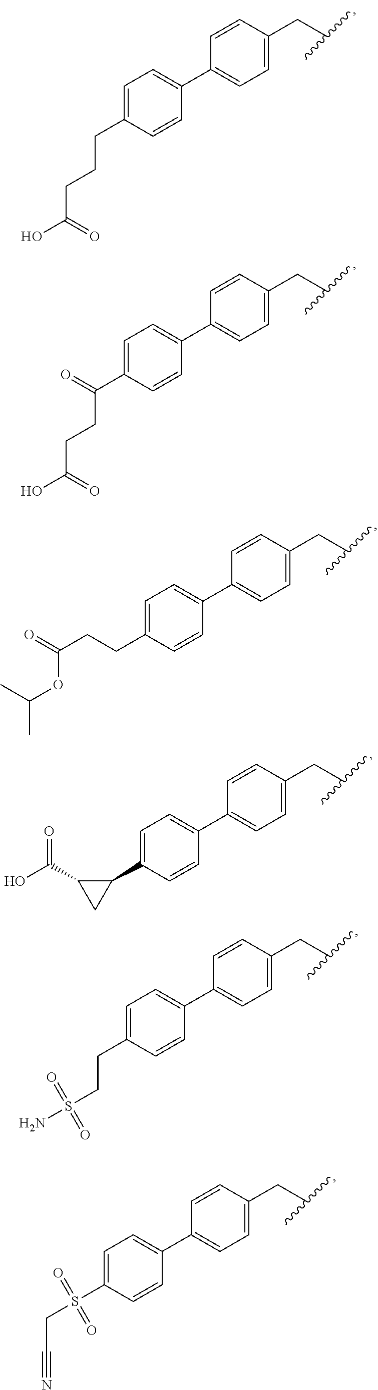 [SMILES: *Cc1ccc(-c2ccc(CCCC(=O)O)cc2)cc1 ; *Cc1ccc(-c2ccc(C(=O)CCC(=O)O)cc2)cc1 ; *Cc1ccc(-c2ccc(CCC(=O)OC(C)C)cc2)cc1 ; *Cc1ccc(-c2ccc([C@H]3C[C@@H]3C(=O)O)cc2)cc1 ; *Cc1ccc(-c2ccc(CCS(N)(=O)=O)cc2)cc1 ; *Cc1ccc(-c2ccc(S(=O)(=O)CC#N)cc2)cc1]

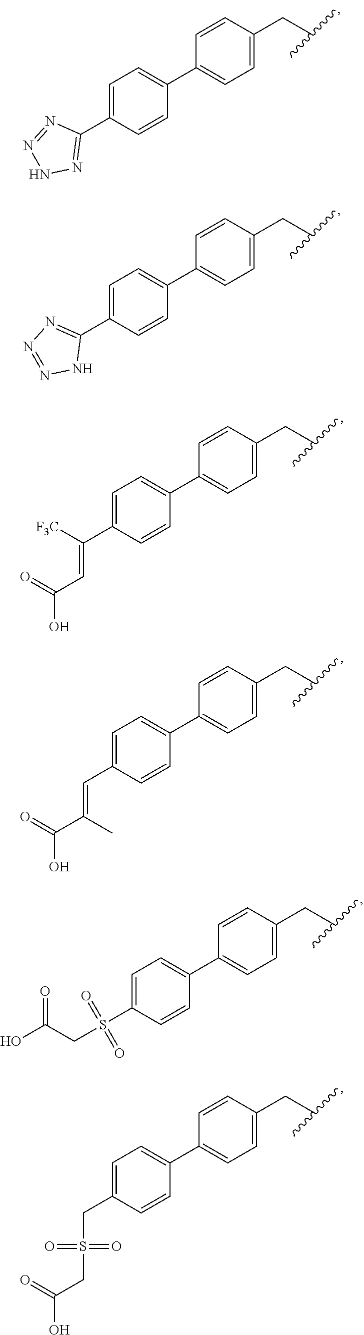 [SMILES: *Cc1ccc(-c2ccc(-c3nn[nH]n3)cc2)cc1 ; *Cc1ccc(-c2ccc(-c3nnn[nH]3)cc2)cc1 ; *Cc1ccc(-c2ccc(/C(=C/C(=O)O)C(F)(F)F)cc2)cc1 ; *Cc1ccc(-c2ccc(/C=C(\C)C(=O)O)cc2)cc1 ; *Cc1ccc(-c2ccc(S(=O)(=O)CC(=O)O)cc2)cc1 ; *Cc1ccc(-c2ccc(CS(=O)(=O)CC(=O)O)cc2)cc1]

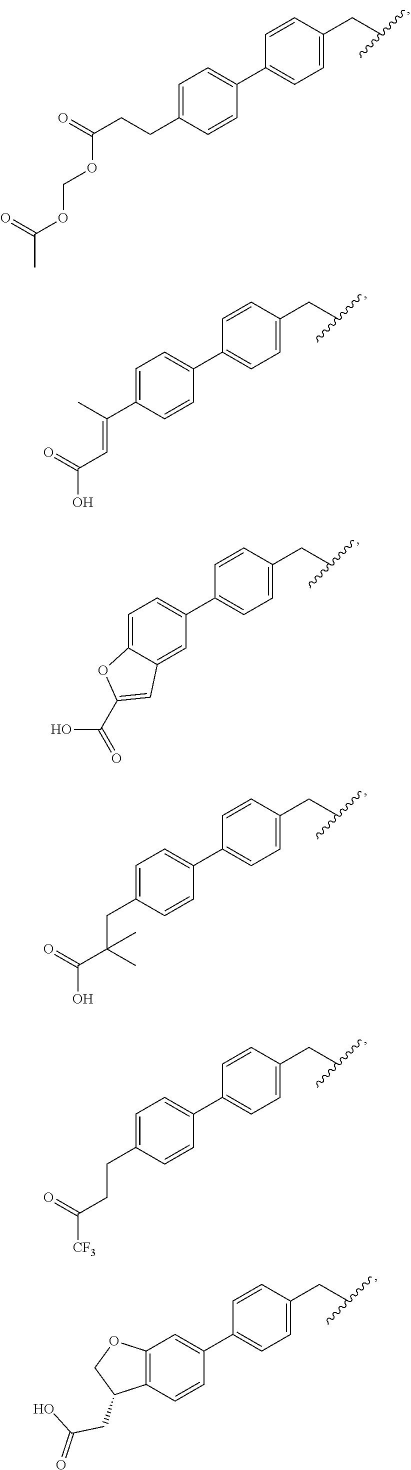 [SMILES: *Cc1ccc(-c2ccc(CCC(=O)OCOC(C)=O)cc2)cc1 ; *Cc1ccc(-c2ccc(/C(C)=C/C(=O)O)cc2)cc1 ; *Cc1ccc(-c2ccc3oc(C(=O)O)cc3c2)cc1 ; *Cc1ccc(-c2ccc(CC(C)(C)C(=O)O)cc2)cc1 ; *Cc1ccc(-c2ccc(CCC(=O)C(F)(F)F)cc2)cc1 ; *Cc1ccc(-c2ccc3c(c2)OC[C@H]3CC(=O)O)cc1]

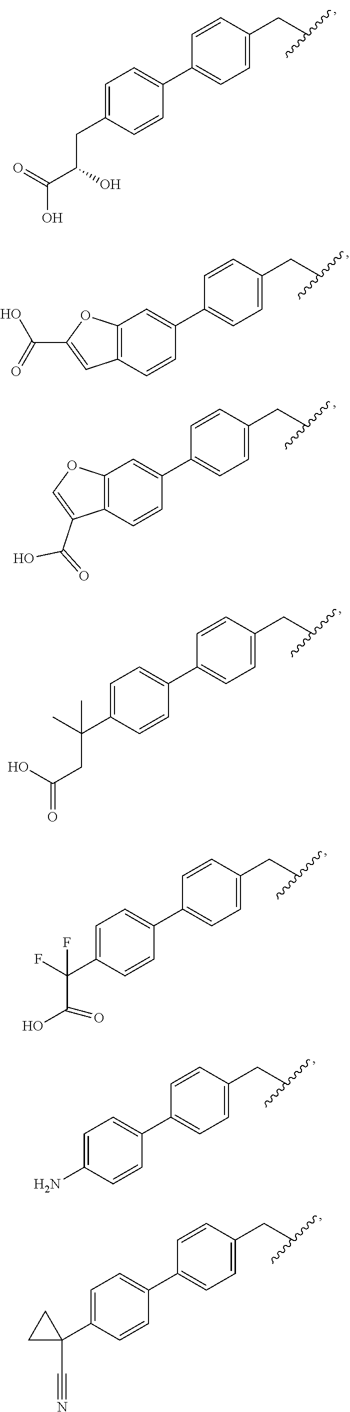

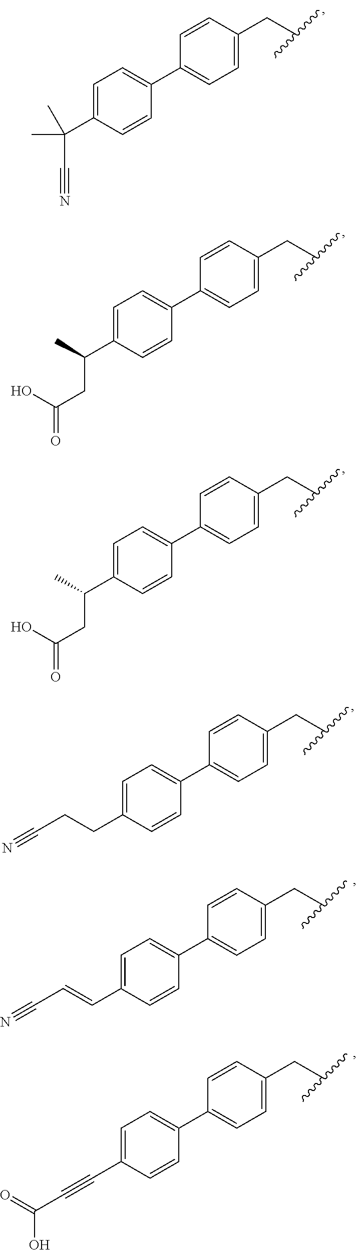 [SMILES: *Cc1ccc(-c2ccc(C(C)(C)C#N)cc2)cc1 ; *Cc1ccc(-c2ccc([C@H](C)CC(=O)O)cc2)cc1 ; *Cc1ccc(-c2ccc([C@@H](C)CC(=O)O)cc2)cc1 ; *Cc1ccc(-c2ccc(CCC#N)cc2)cc1 ; *Cc1ccc(-c2ccc(/C=C/C#N)cc2)cc1 ; *Cc1ccc(-c2ccc(C#CC(=O)O)cc2)cc1]

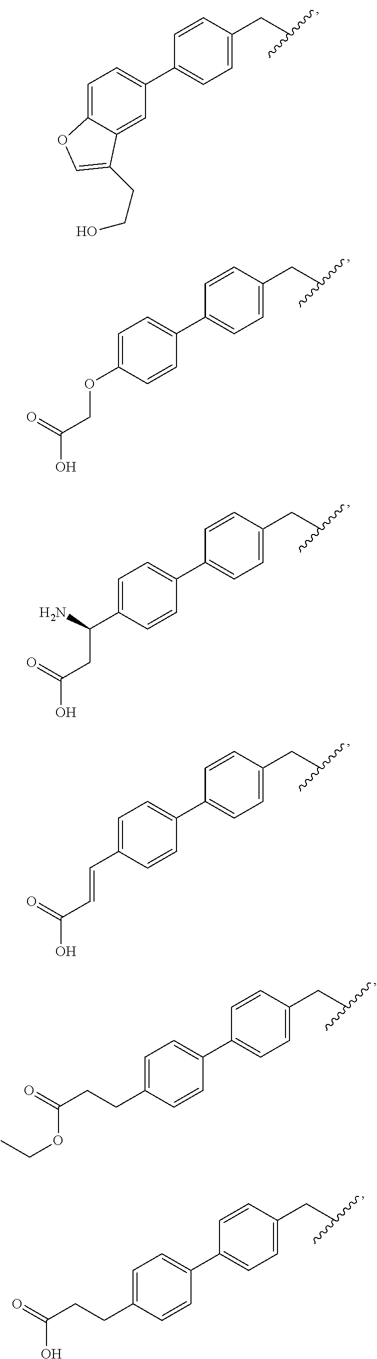 [SMILES: *Cc1ccc(-c2ccc3occ(CCO)c3c2)cc1 ; *Cc1ccc(-c2ccc(OCC(=O)O)cc2)cc1 ; *Cc1ccc(-c2ccc([C@H](N)CC(=O)O)cc2)cc1 ; *Cc1ccc(-c2ccc(/C=C/C(=O)O)cc2)cc1 ; *Cc1ccc(-c2ccc(CCC(=O)OCC)cc2)cc1 ; *Cc1ccc(-c2ccc(CCC(=O)O)cc2)cc1]

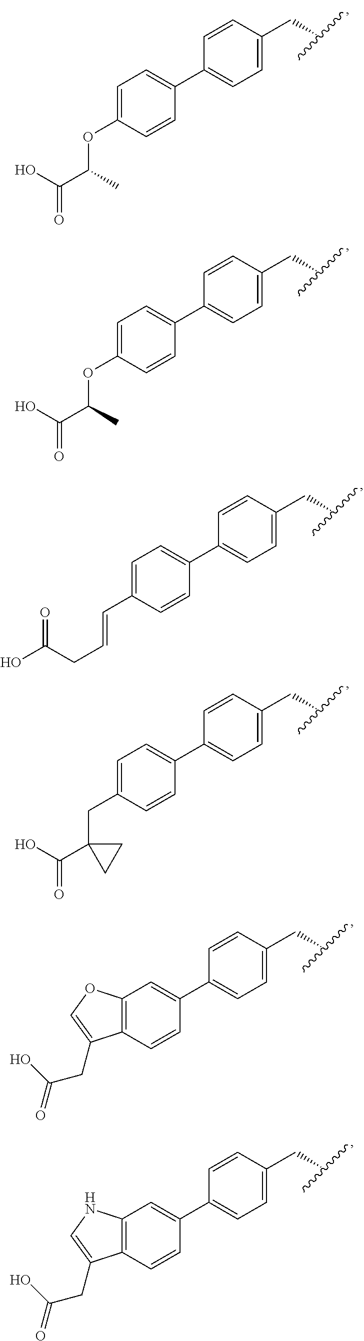

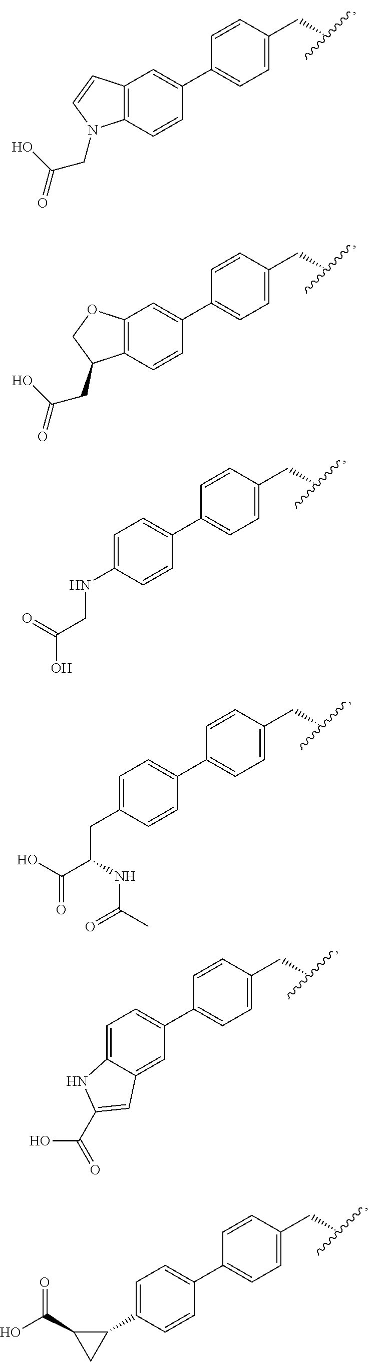

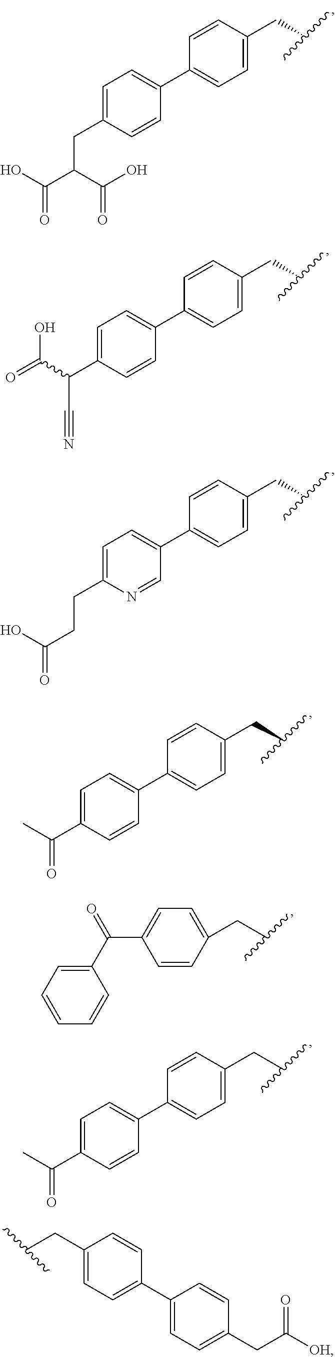

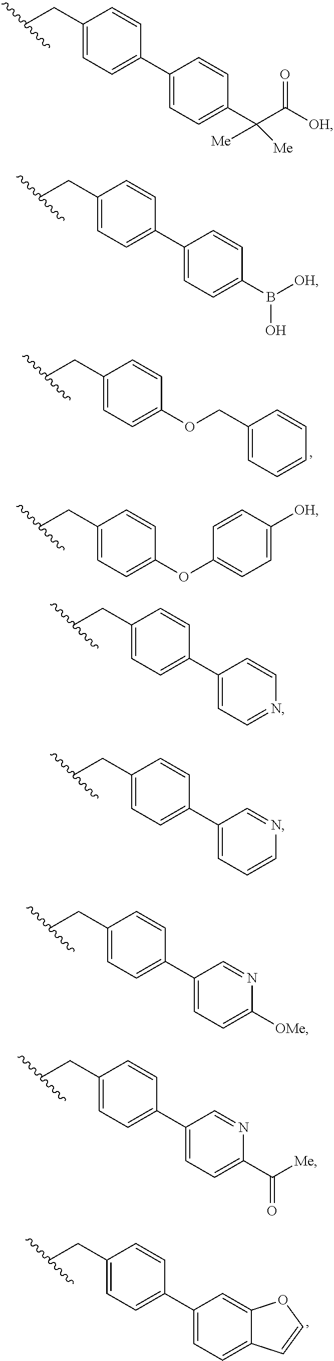

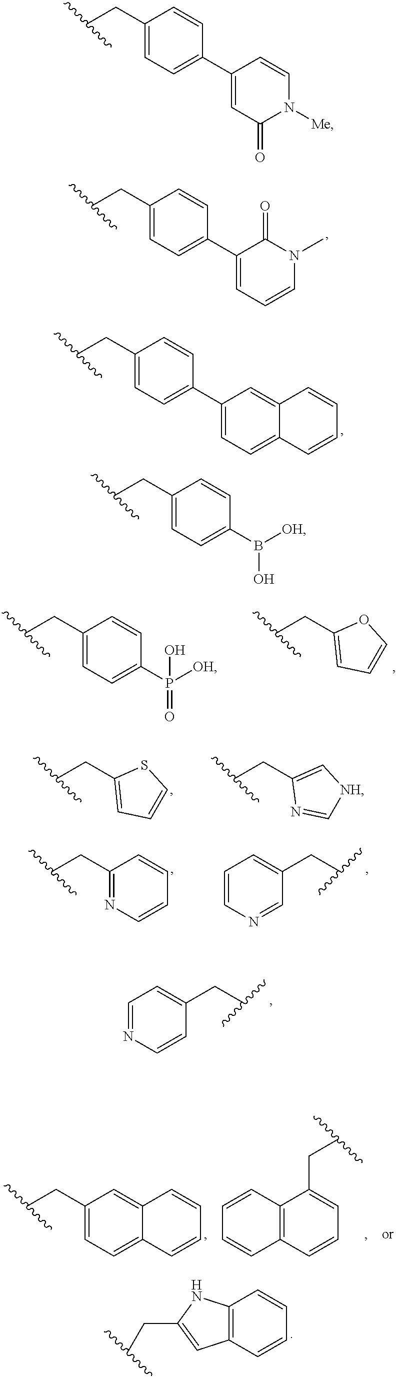

29. The compound of any one of claims 1-8 or 19, wherein R¹ is substituted or unsubstituted alkenyl.

30. The compound of any one of claims 1-29, wherein y is 0.

31. The compound of any one of claims 1-29, wherein y is 1.

32. The compound of any one of claims 3-6 or 15-31, wherein R² is hydrogen.

33. The compound of any one of claims 3-7 or 15-31, wherein R² is substituted or unsubstituted C₁₋₆ alkyl.

34. The compound of any one of claims 1-33, wherein the moiety 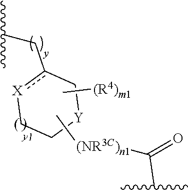
is of formula

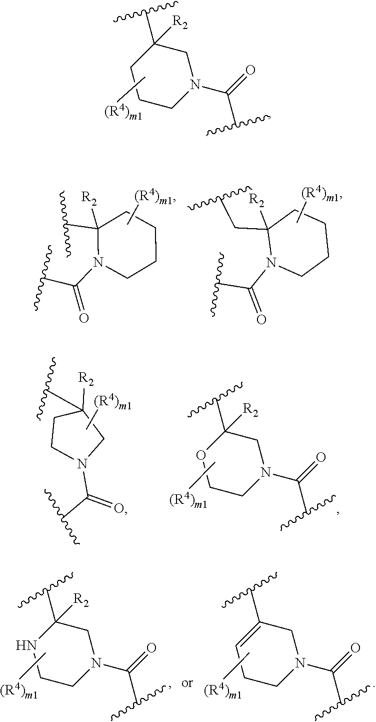

35. The compound of any one of claims 1-33, wherein the moiety 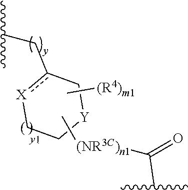
is of formula:

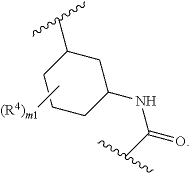

36. The compound of any one of claims 1-35, wherein at least one instance of R⁵ is hydrogen.

37. The compound of any one of claims 1-36, wherein at least one instance of R⁵ is C₁₋₆ alkyl optionally substituted with halogen, —OR^{c1}, or —N(R^{c2})₂; and
R^{c1} is hydrogen or C₁₋₆ alkyl optionally substituted with —N(R^{c2})₂, and
each instance of R^{c2} is independently hydrogen, optionally substituted acyl, optionally substituted alkyl, optionally substituted alkenyl, optionally substituted alkynyl, or a nitrogen protecting group.

38. The compound of any one of claims 1-37, wherein at least one instance of R⁵ is methyl or of formula

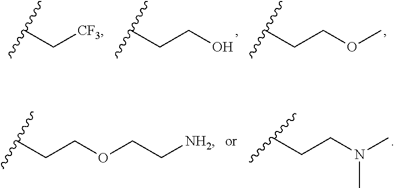

39. The compound of any one of claims 1-36, wherein the moiety 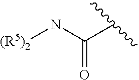
is

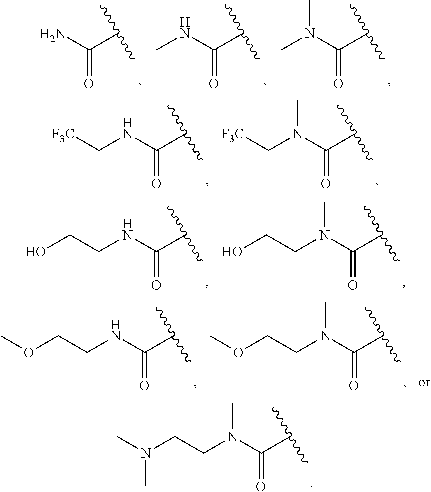

40. The compound of any one of claims 1, 4, 7, 9, 11, 13, or 15-35, wherein R^{5A} is hydrogen.

41. The compound of any one of claims 1, 4, 7, 9, 11, 13, or 15-35, wherein R^{5A} is optionally substituted C₁₋₆ alkyl.

42. The compound of any one of claims 1, 4, 7, 9, 11, 13, 15-35, or 41, wherein R^{5A} is methyl.

43. The compound of any one of claims 1-42, wherein each of R^{A}, R^{B}, R^{C}, and R^{D} is hydrogen.

44. The compound of any one of claims 1-43, wherein x is 0.

45. The compound of any one of claims 1-44, wherein the compound is of formula: 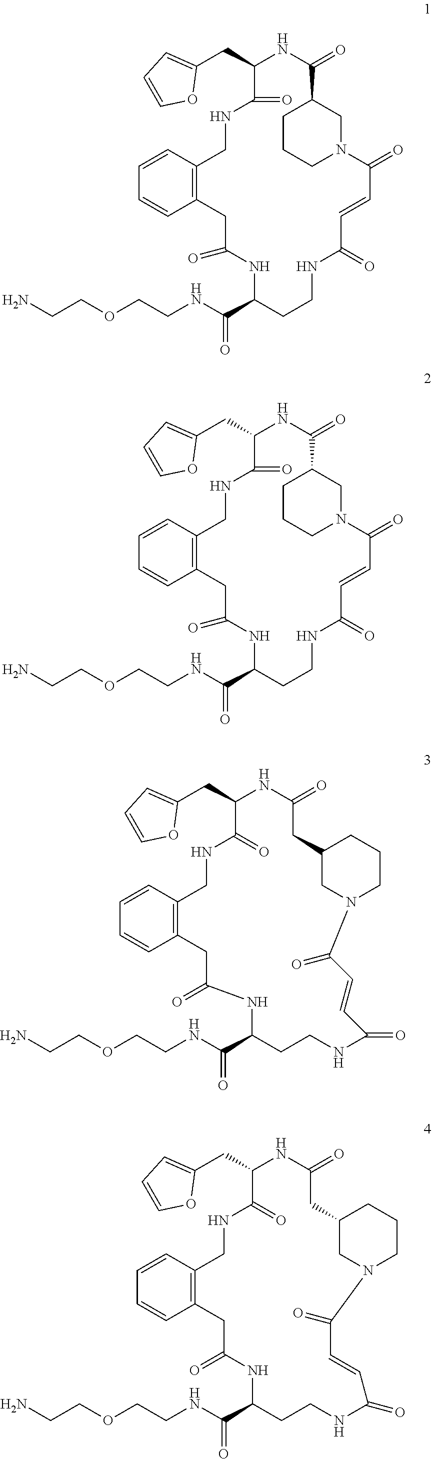 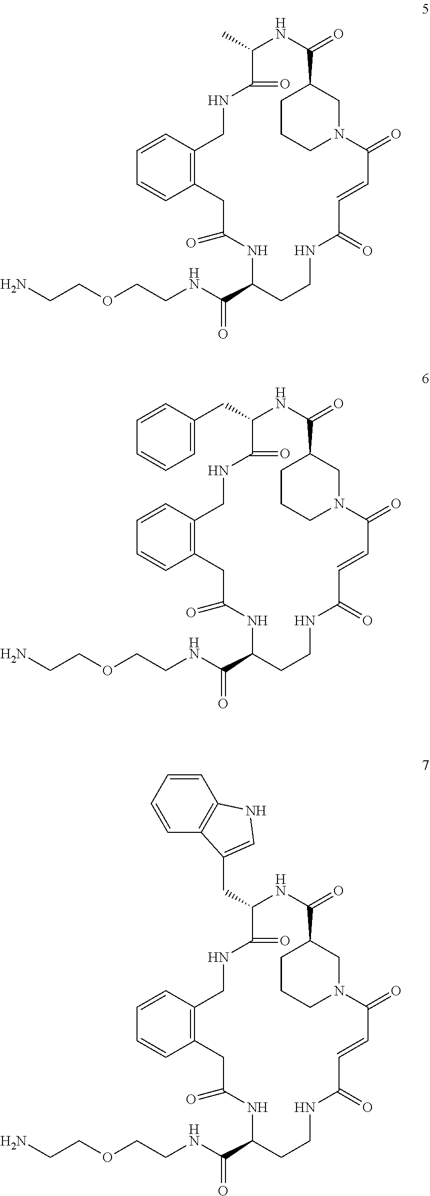 [SMILES: C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccccc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2c[nH]c3ccccc23)C(=O)NCc2ccccc2CC(=O)N1] 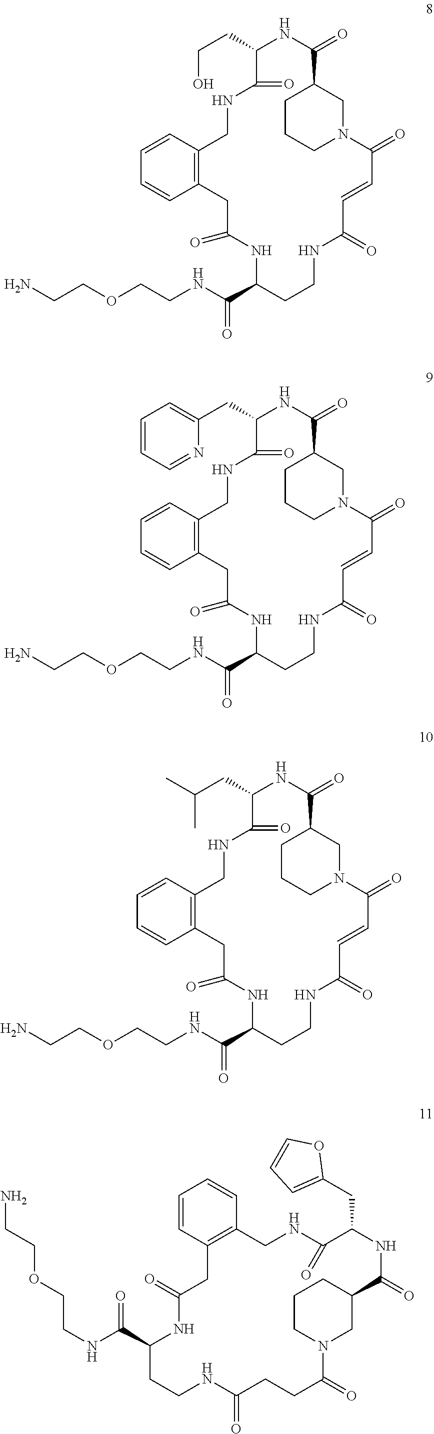 [SMILES: NCCOCCNC(=O)[C@@H]1CCNC(=O)/C=C/C(=O)N2CCC[C@H](C2)C(=O)N[C@@H](CCO)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccccn2)C(=O)NCc2ccccc2CC(=O)N1 ; CC(C)C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)CCC(=O)N2CCC[C@H](C2)C(=O)N[C@@H](Cc2ccco2)C(=O)NCc2ccccc2CC(=O)N1] 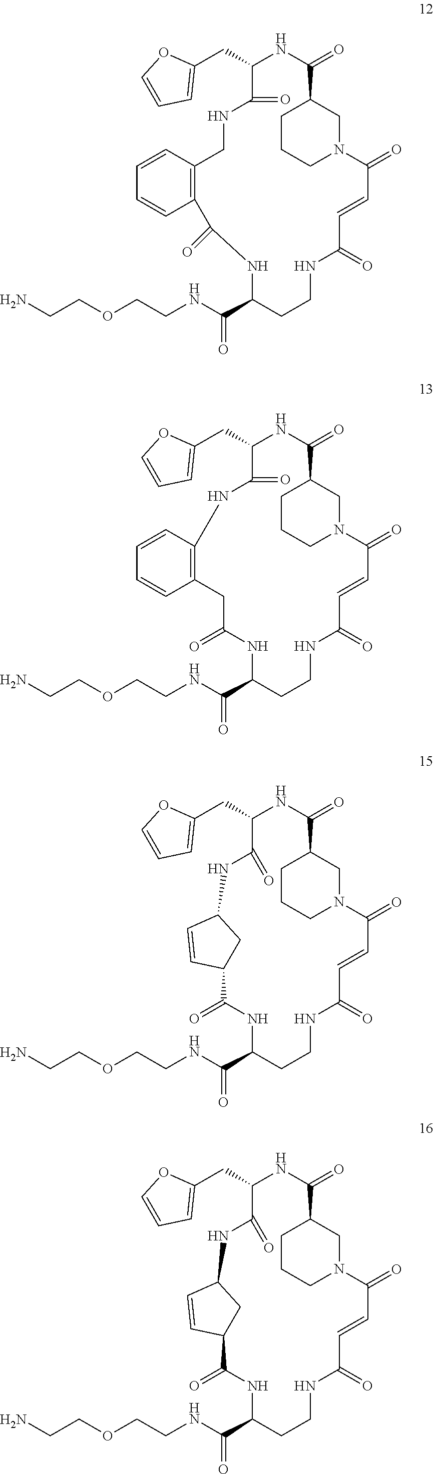 [SMILES: NCCOCCNC(=O)[C@@H]1CCNC(=O)/C=C/C(=O)N2CCC[C@H](C2)C(=O)N[C@@H](Cc2ccco2)C(=O)NCc2ccccc2C(=O)N1 ; NCCOCCNC(=O)[C@@H]1CCNC(=O)/C=C/C(=O)N2CCC[C@H](C2)C(=O)N[C@@H](Cc2ccco2)C(=O)Nc2ccccc2CC(=O)N1 ; NCCOCCNC(=O)[C@@H]1CCNC(=O)/C=C/C(=O)N2CCC[C@H](C2)C(=O)N[C@@H](Cc2ccco2)C(=O)N[C@H]2C=C[C@H](C2)C(=O)N1 ; NCCOCCNC(=O)[C@@H]1CCNC(=O)/C=C/C(=O)N2CCC[C@H](C2)C(=O)N[C@@H](Cc2ccco2)C(=O)N[C@@H]2C=C[C@@H](C2)C(=O)N1] 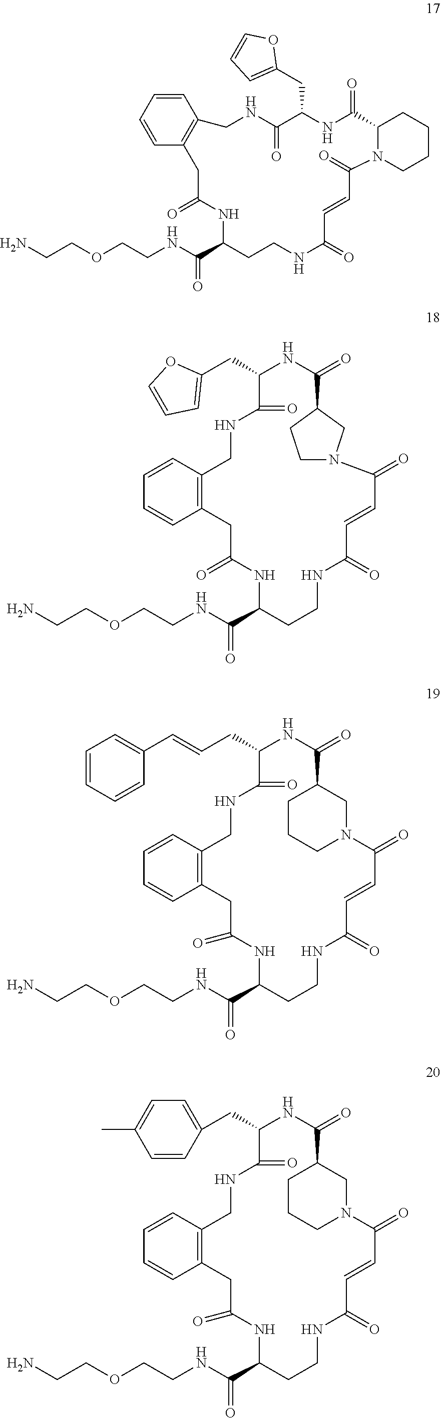 [SMILES: NCCOCCNC(=O)[C@@H]1CCNC(=O)/C=C/C(=O)N2CCCC[C@H]2C(=O)N[C@@H](Cc2ccco2)C(=O)NCc2ccccc2CC(=O)N1 ; NCCOCCNC(=O)[C@@H]1CCNC(=O)/C=C/C(=O)N2CC[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@H](C2)C(=O)N[C@@H](C/C=C/c2ccccc2)C(=O)NCc2ccccc2CC(=O)N1 ; Cc1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1] 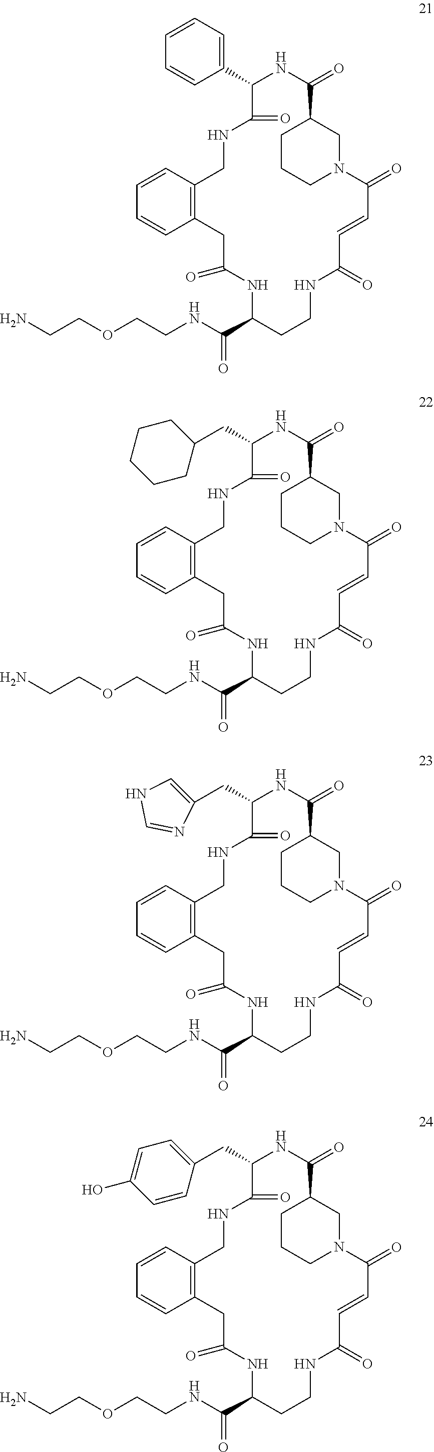 [SMILES: NCCOCCNC(=O)[C@@H]1CCNC(=O)/C=C/C(=O)N2CCC[C@H](C2)C(=O)N[C@@H](c2ccccc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](CC2CCCCC2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2c[nH]cn2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(O)cc2)C(=O)NCc2ccccc2CC(=O)N1] 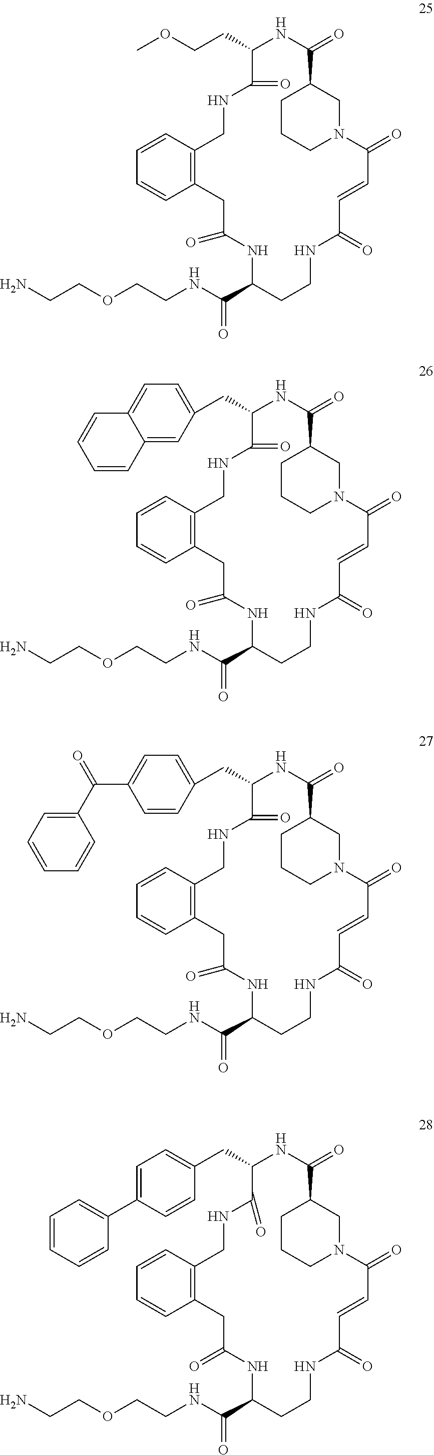 [SMILES: COCC[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccc3ccccc3c2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(C(=O)c3ccccc3)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(-c3ccccc3)cc2)C(=O)NCc2ccccc2CC(=O)N1] 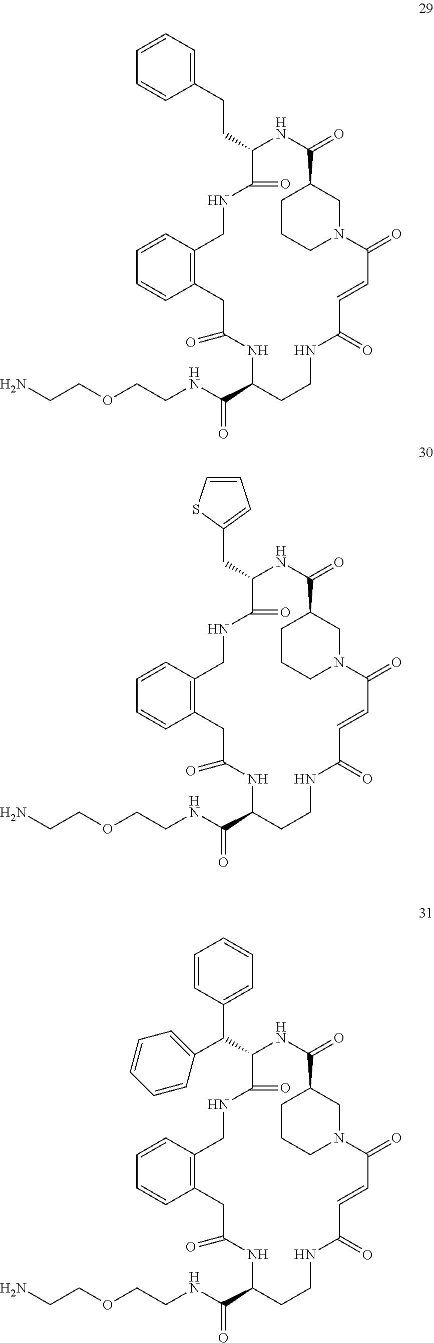 [SMILES: NCCOCCNC(=O)[C@@H]1CCNC(=O)/C=C/C(=O)N2CCC[C@H](C2)C(=O)N[C@@H](CCc2ccccc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2cccs2)C(=O)NCc2ccccc2CC(=O)N1 ; NCCOCCNC(=O)[C@@H]1CCNC(=O)/C=C/C(=O)N2CCC[C@H](C2)C(=O)N[C@@H](C(c2ccccc2)c2ccccc2)C(=O)NCc2ccccc2CC(=O)N1] 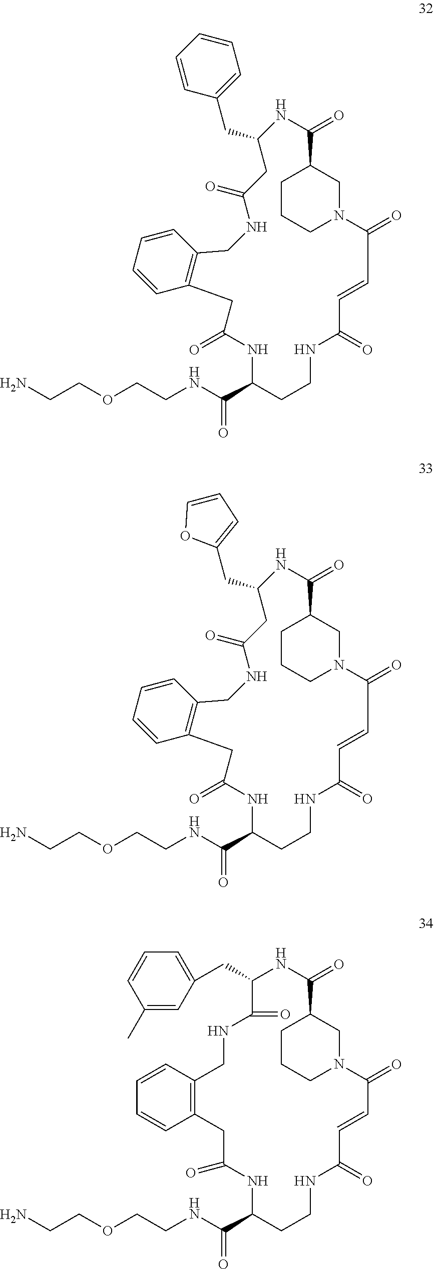 [SMILES: NCCOCCNC(=O)[C@@H]1CCNC(=O)/C=C/C(=O)N2CCC[C@H](C2)C(=O)N[C@@H](Cc2ccccc2)CC(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccco2)CC(=O)NCc2ccccc2CC(=O)N1 ; Cc1cccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)c1] 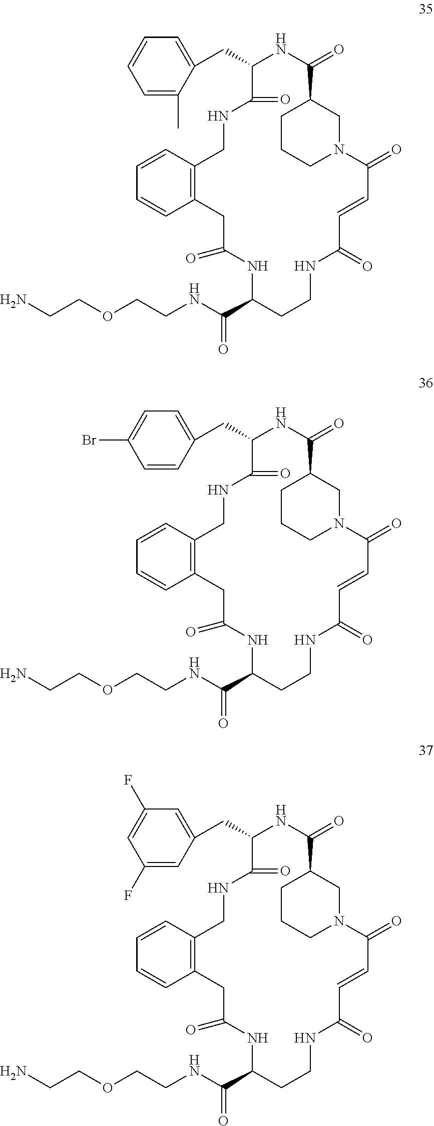 [SMILES: Cc1ccccc1C[C@@H]1NC(=O)[C@@H]2CCCN(C2)C(=O)/C=C/C(=O)NCC[C@@H](C(=O)NCCOCCN)NC(=O)Cc2ccccc2CNC1=O ; NCCOCCNC(=O)[C@@H]1CCNC(=O)/C=C/C(=O)N2CCC[C@H](C2)C(=O)N[C@@H](Cc2ccc(Br)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2cc(F)cc(F)c2)C(=O)NCc2ccccc2CC(=O)N1] 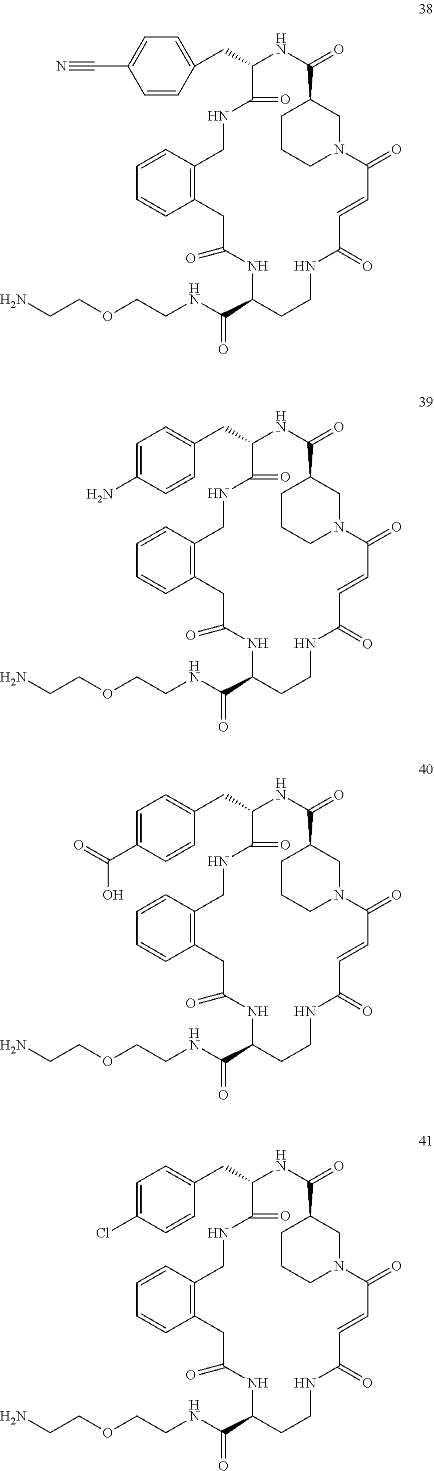 [SMILES: N#Cc1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@H](C2)C(=O)N[C@@H](Cc2ccc(N)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(C(=O)O)cc2)C(=O)NCc2ccccc2CC(=O)N1 ; NCCOCCNC(=O)[C@@H]1CCNC(=O)/C=C/C(=O)N2CCC[C@H](C2)C(=O)N[C@@H](Cc2ccc(Cl)cc2)C(=O)NCc2ccccc2CC(=O)N1] 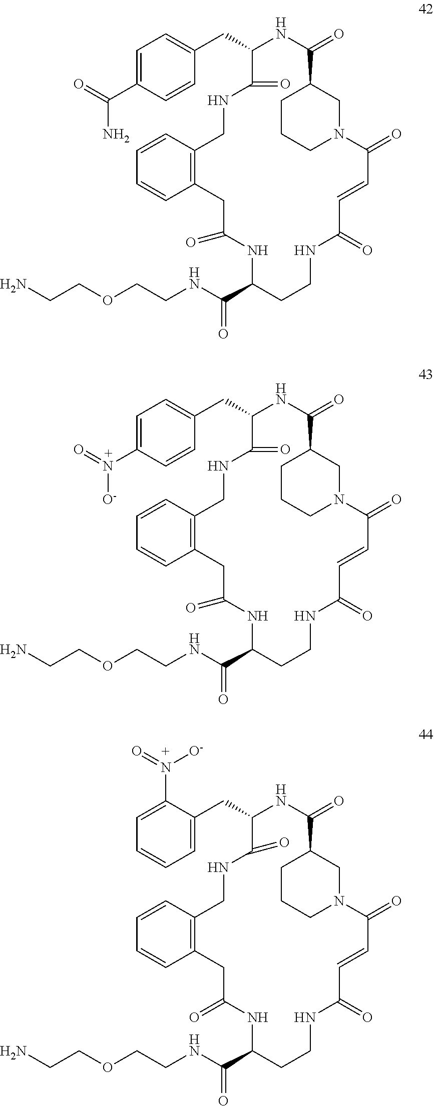 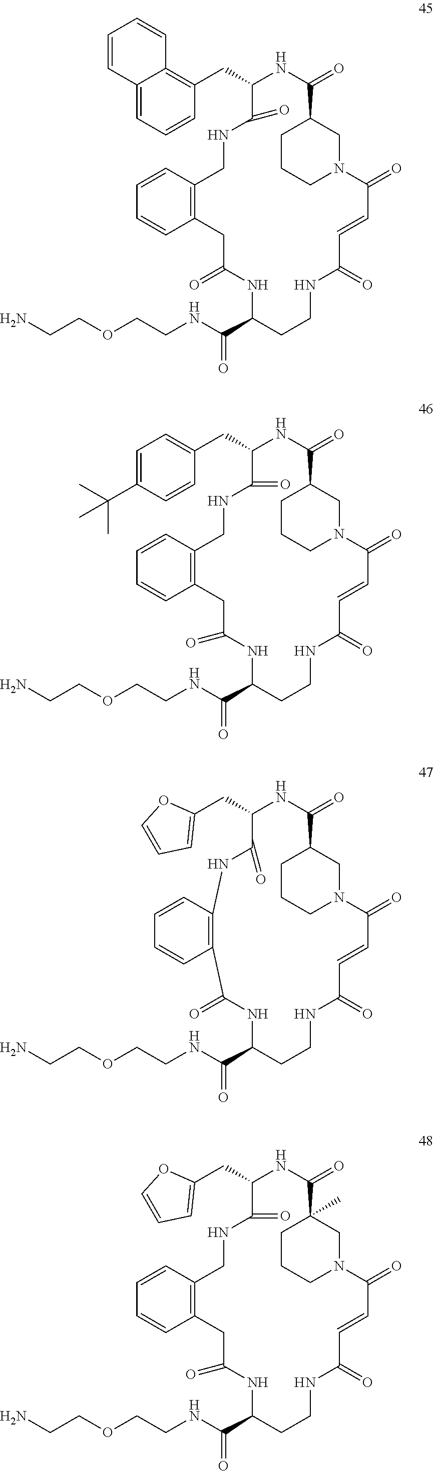 [SMILES: NCCOCCNC(=O)[C@@H]1CCNC(=O)/C=C/C(=O)N2CCC[C@H](C2)C(=O)N[C@@H](Cc2cccc3ccccc23)C(=O)NCc2ccccc2CC(=O)N1 ; CC(C)(C)c1ccc(C[C@@H]2NC(=O)[C@@H]3CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@H](C2)C(=O)N[C@@H](Cc2ccco2)C(=O)Nc2ccccc2C(=O)N1 ; C[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O] 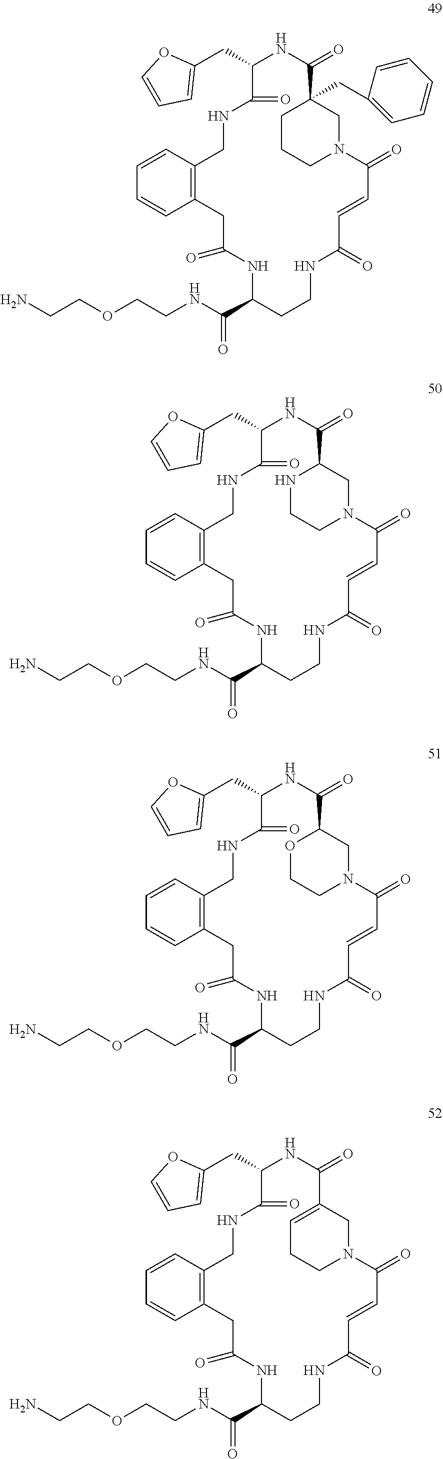 [SMILES: NCCOCCNC(=O)[C@@H]1CCNC(=O)/C=C/C(=O)N2CCC[C@](Cc3ccccc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCN[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCO[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC=C(C2)C(=O)N[C@@H](Cc2ccco2)C(=O)NCc2ccccc2CC(=O)N1] 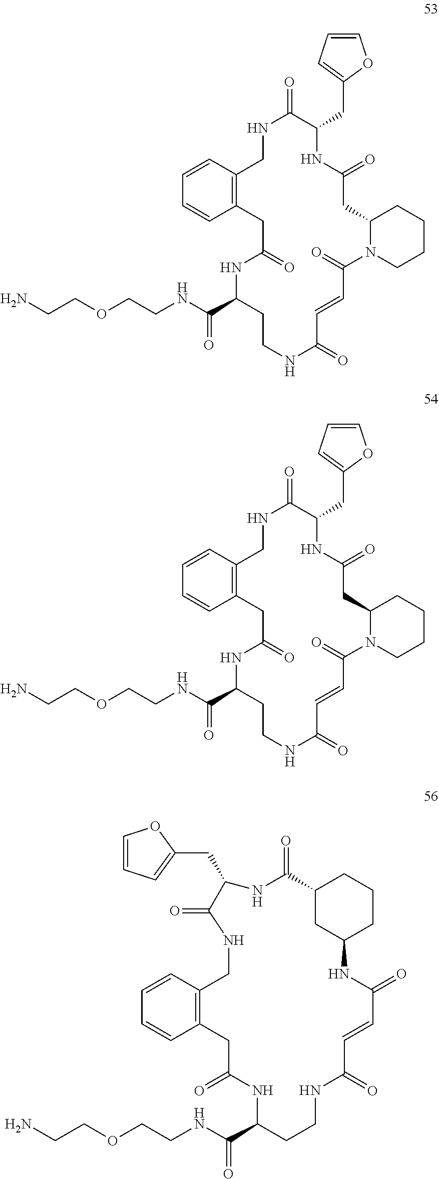 [SMILES: NCCOCCNC(=O)[C@@H]1CCNC(=O)/C=C/C(=O)N2CCCC[C@H]2CC(=O)N[C@@H](Cc2ccco2)C(=O)NCc2ccccc2CC(=O)N1 ; NCCOCCNC(=O)[C@@H]1CCNC(=O)/C=C/C(=O)N2CCCC[C@@H]2CC(=O)N[C@@H](Cc2ccco2)C(=O)NCc2ccccc2CC(=O)N1 ; NCCOCCNC(=O)[C@@H]1CCNC(=O)/C=C/C(=O)N[C@@H]2CCC[C@H](C2)C(=O)N[C@@H](Cc2ccco2)C(=O)NCc2ccccc2CC(=O)N1] 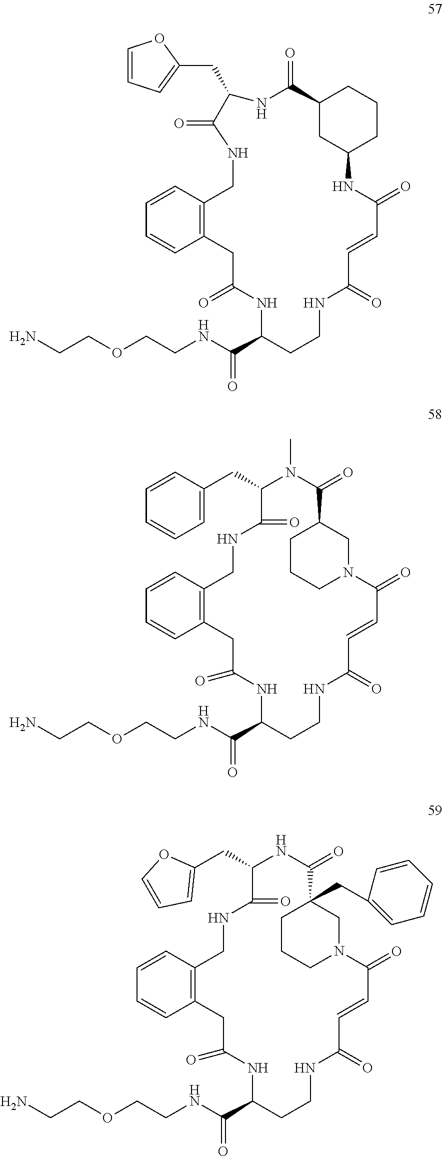 [SMILES: NCCOCCNC(=O)[C@@H]1CCNC(=O)/C=C/C(=O)N[C@@H]2CCC[C@@H](C2)C(=O)N[C@@H](Cc2ccco2)C(=O)NCc2ccccc2CC(=O)N1 ; CN1C(=O)[C@@H]2CCCN(C2)C(=O)/C=C/C(=O)NCC[C@@H](C(=O)NCCOCCN)NC(=O)Cc2ccccc2CNC(=O)[C@@H]1Cc1ccccc1 ; NCCOCCNC(=O)[C@@H]1CCNC(=O)/C=C/C(=O)N2CCC[C@@](Cc3ccccc3)(C2)C(=O)N[C@@H](Cc2ccco2)C(=O)NCc2ccccc2CC(=O)N1] 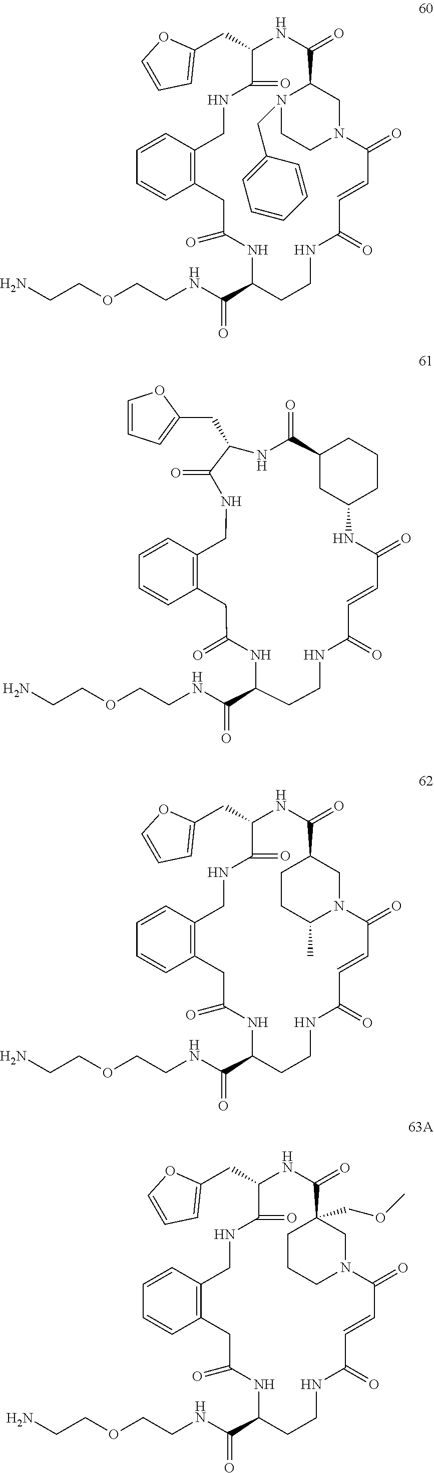 [SMILES: NCCOCCNC(=O)[C@@H]1CCNC(=O)/C=C/C(=O)N2CCN(Cc3ccccc3)[C@H](C2)C(=O)N[C@@H](Cc2ccco2)C(=O)NCc2ccccc2CC(=O)N1 ; NCCOCCNC(=O)[C@@H]1CCNC(=O)/C=C/C(=O)N[C@H]2CCC[C@@H](C2)C(=O)N[C@@H](Cc2ccco2)C(=O)NCc2ccccc2CC(=O)N1 ; C[C@@H]1CC[C@@H]2CN1C(=O)/C=C/C(=O)NCC[C@@H](C(=O)NCCOCCN)NC(=O)Cc1ccccc1CNC(=O)[C@H](Cc1ccco1)NC2=O ; COC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O] 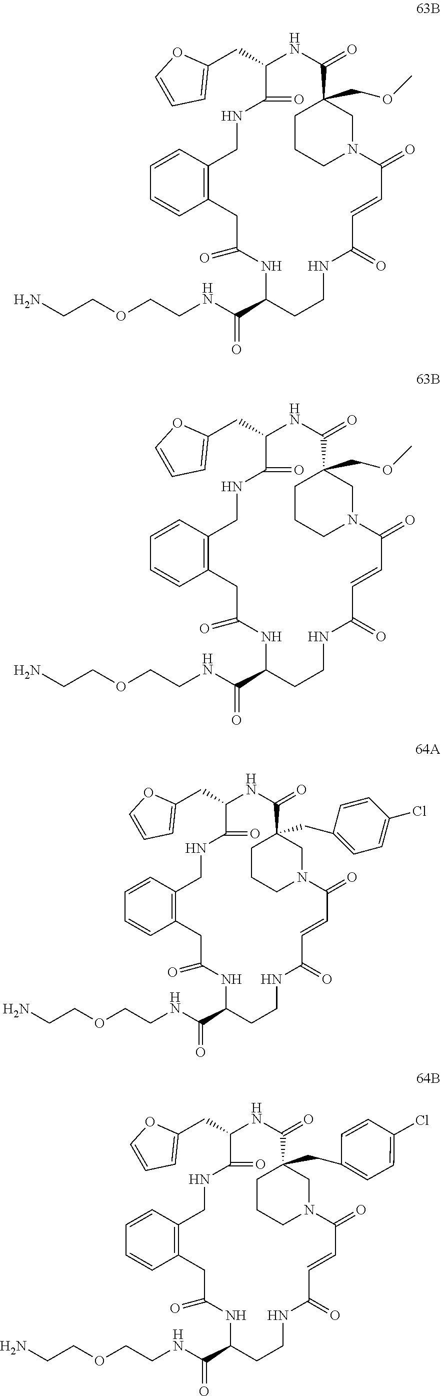 [SMILES: COC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; COC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; NCCOCCNC(=O)[C@@H]1CCNC(=O)/C=C/C(=O)N2CCC[C@](Cc3ccc(Cl)cc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(Cl)cc3)(C2)C(=O)N[C@@H](Cc2ccco2)C(=O)NCc2ccccc2CC(=O)N1] 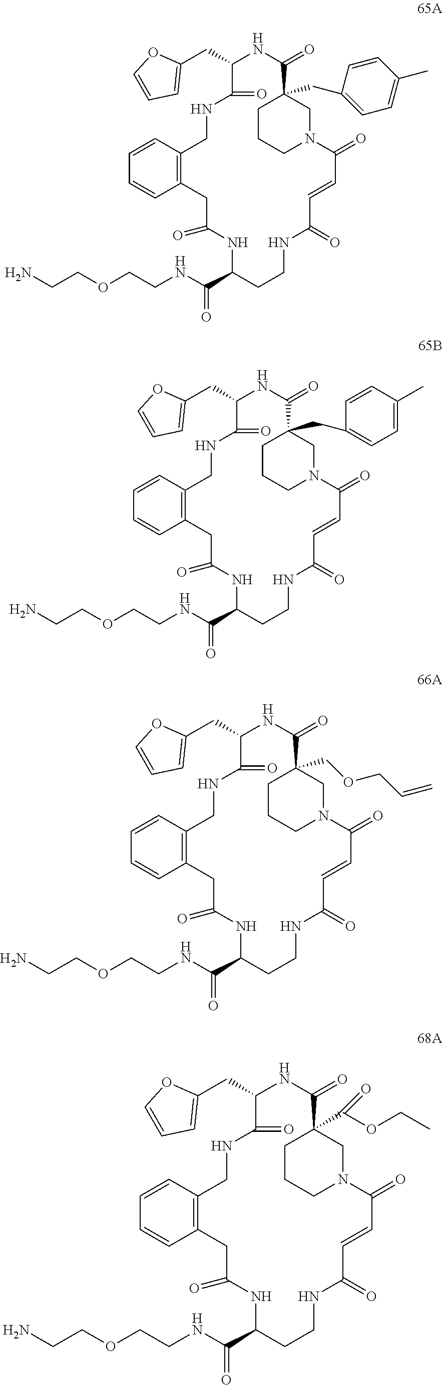 [SMILES: Cc1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; Cc1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; C=CCOC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CCOC(=O)[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O] 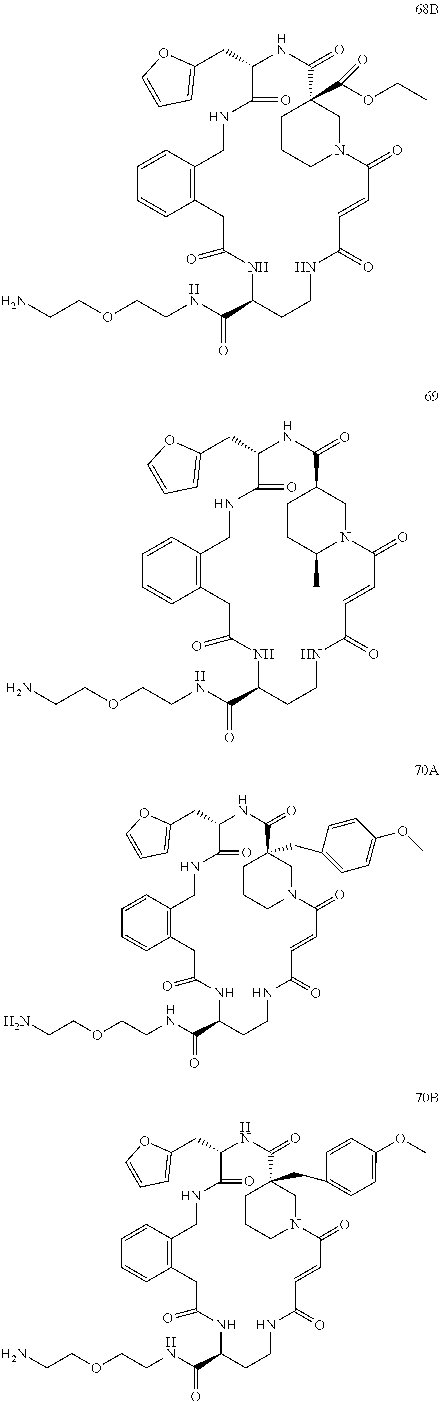 [SMILES: CCOC(=O)[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; C[C@H]1CC[C@@H]2CN1C(=O)/C=C/C(=O)NCC[C@@H](C(=O)NCCOCCN)NC(=O)Cc1ccccc1CNC(=O)[C@H](Cc1ccco1)NC2=O ; COc1ccc(C[C@@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1 ; COc1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1] 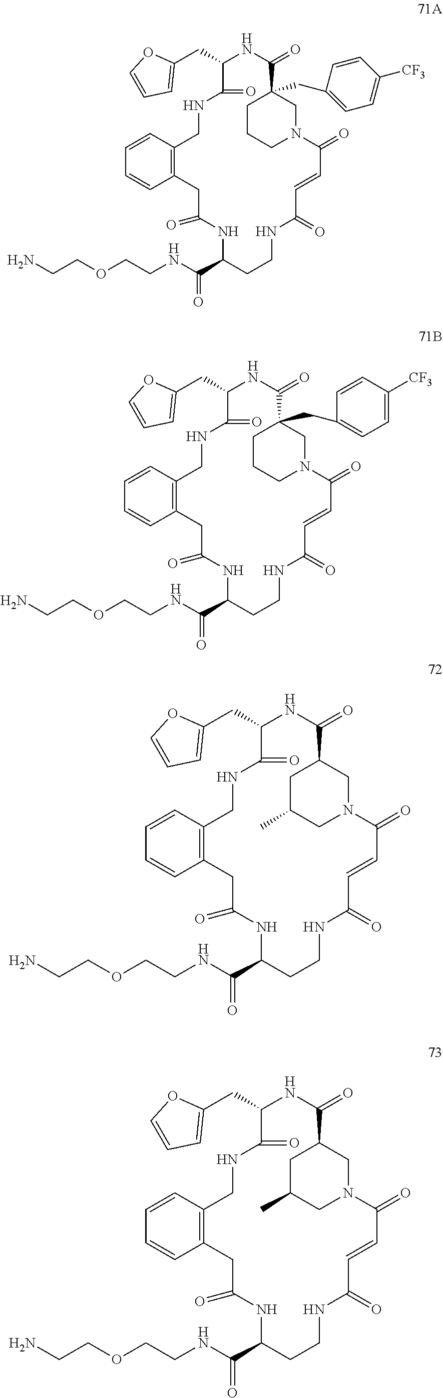 [SMILES: NCCOCCNC(=O)[C@@H]1CCNC(=O)/C=C/C(=O)N2CCC[C@](Cc3ccc(C(F)(F)F)cc3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(C(F)(F)F)cc3)(C2)C(=O)N[C@@H](Cc2ccco2)C(=O)NCc2ccccc2CC(=O)N1 ; C[C@@H]1C[C@@H]2CN(C1)C(=O)/C=C/C(=O)NCC[C@@H](C(=O)NCCOCCN)NC(=O)Cc1ccccc1CNC(=O)[C@H](Cc1ccco1)NC2=O ; C[C@H]1C[C@@H]2CN(C1)C(=O)/C=C/C(=O)NCC[C@@H](C(=O)NCCOCCN)NC(=O)Cc1ccccc1CNC(=O)[C@H](Cc1ccco1)NC2=O] 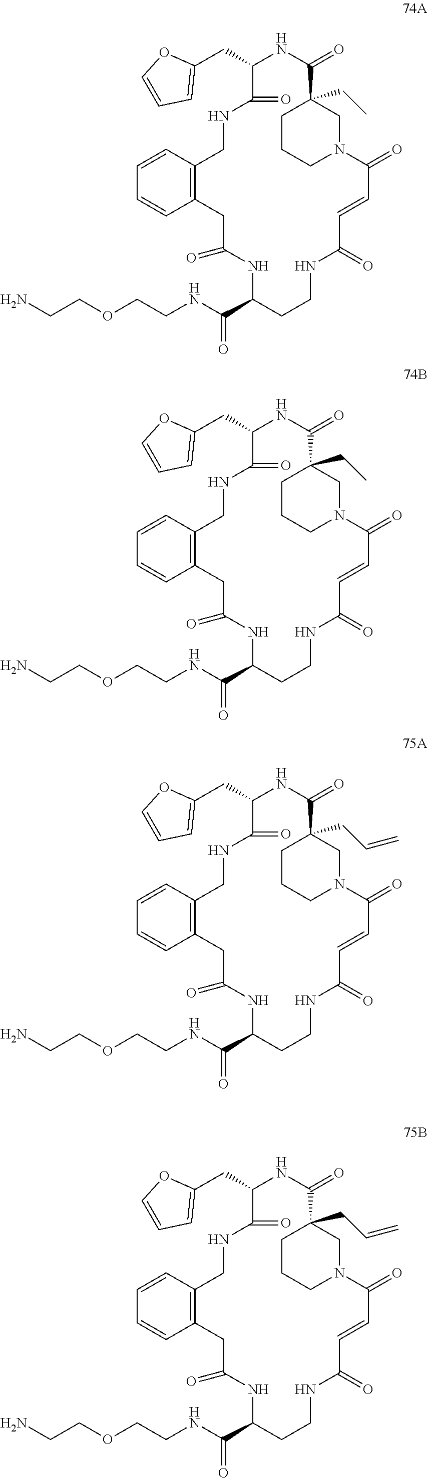 [SMILES: CC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; CC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; C=CC[C@@]12CCCN(C1)C(=O)/C=C/C(=O)NCC[C@@H](C(=O)NCCOCCN)NC(=O)Cc1ccccc1CNC(=O)[C@H](Cc1ccco1)NC2=O ; C=CC[C@]12CCCN(C1)C(=O)/C=C/C(=O)NCC[C@@H](C(=O)NCCOCCN)NC(=O)Cc1ccccc1CNC(=O)[C@H](Cc1ccco1)NC2=O] 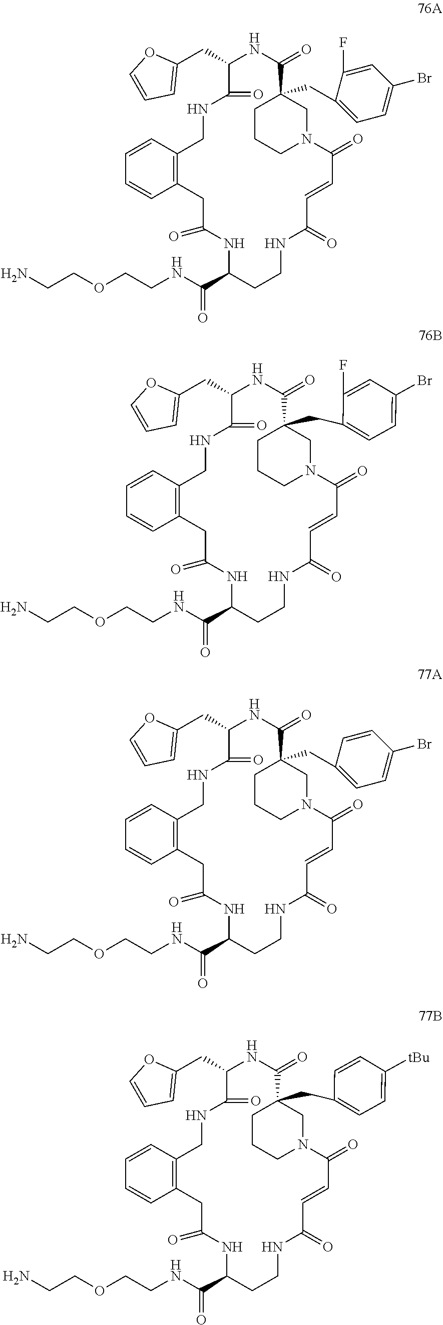 [SMILES: NCCOCCNC(=O)[C@@H]1CCNC(=O)/C=C/C(=O)N2CCC[C@](Cc3ccc(Br)cc3F)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(Br)cc3F)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3ccc(Br)cc3)(C2)C(=O)N[C@@H](Cc2ccco2)C(=O)NCc2ccccc2CC(=O)N1 ; CC(C)(C)c1ccc(C[C@]23CCCN(C2)C(=O)/C=C/C(=O)NCC[C@@H](C(=O)NCCOCCN)NC(=O)Cc2ccccc2CNC(=O)[C@H](Cc2ccco2)NC3=O)cc1] 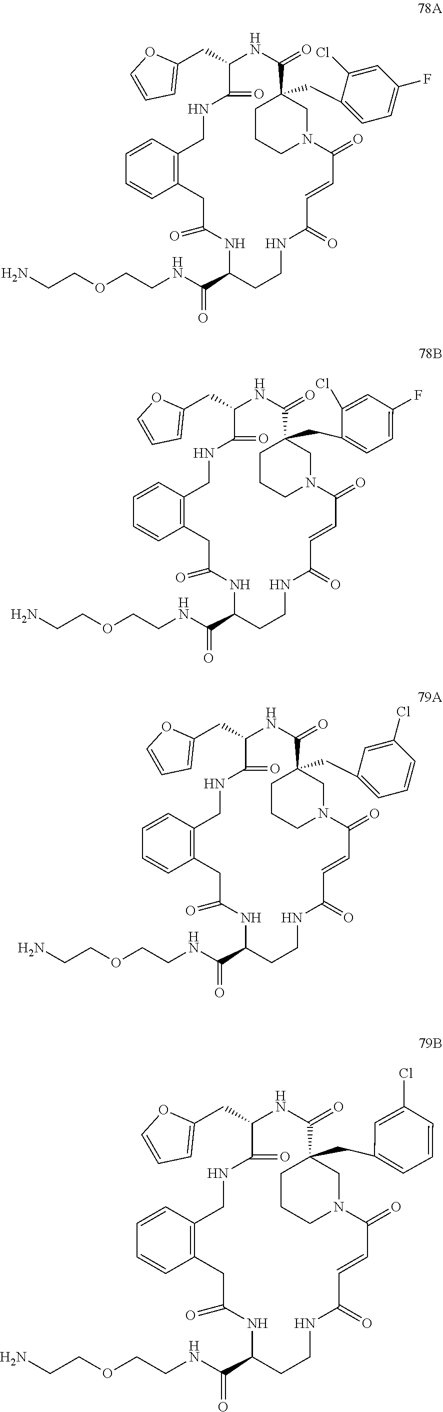 [SMILES: NCCOCCNC(=O)[C@@H]1CCNC(=O)/C=C/C(=O)N2CCC[C@](Cc3ccc(F)cc3Cl)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3ccc(F)cc3Cl)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@](Cc3cccc(Cl)c3)(C2)C(=O)N[C@@H](Cc2ccco2)C(=O)NCc2ccccc2CC(=O)N1 ; NCCOCCNC(=O)[C@@H]1CCNC(=O)/C=C/C(=O)N2CCC[C@@](Cc3cccc(Cl)c3)(C2)C(=O)N[C@@H](Cc2ccco2)C(=O)NCc2ccccc2CC(=O)N1] 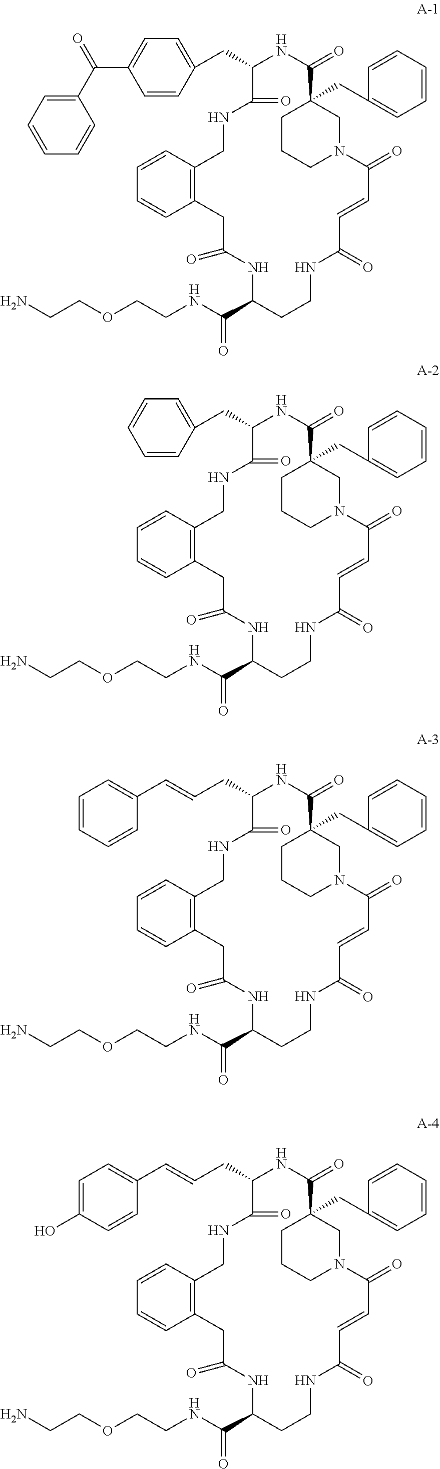 [SMILES: NCCOCCNC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](C/C=C/c2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](C/C=C/c2ccc(O)cc2)C(=O)NCc2ccccc2CC(=O)N1] 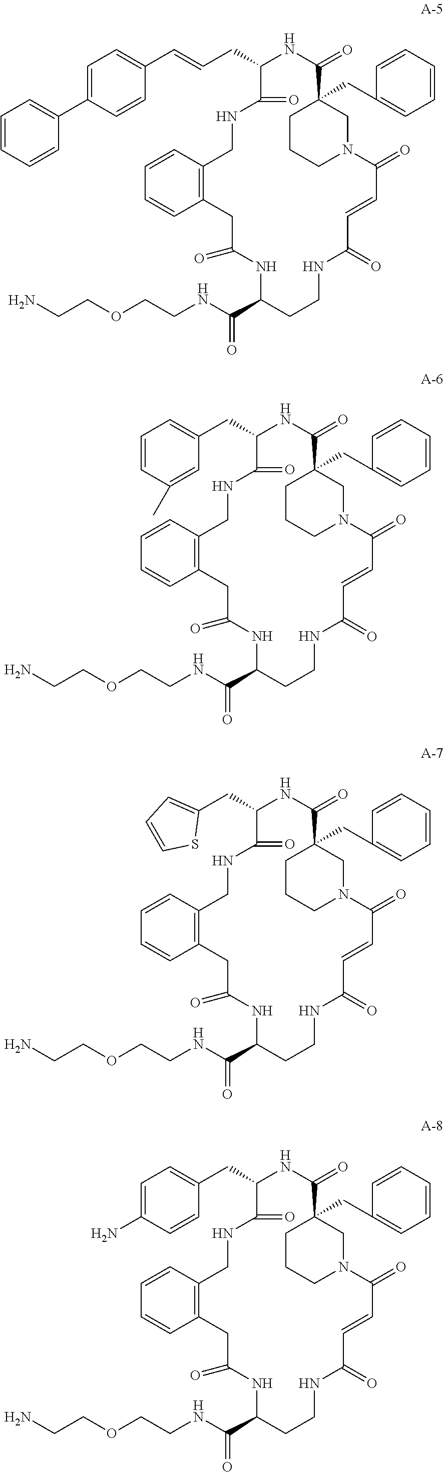 [SMILES: NCCOCCNC(=O)[C@@H]1CCNC(=O)/C=C/C(=O)N2CCC[C@](Cc3ccccc3)(C2)C(=O)N[C@@H](C/C=C/c2ccc(-c3ccccc3)cc2)C(=O)NCc2ccccc2CC(=O)N1 ; Cc1cccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)c1 ; NCCOCCNC(=O)[C@@H]1CCNC(=O)/C=C/C(=O)N2CCC[C@](Cc3ccccc3)(C2)C(=O)N[C@@H](Cc2cccs2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(N)cc2)C(=O)NCc2ccccc2CC(=O)N1] 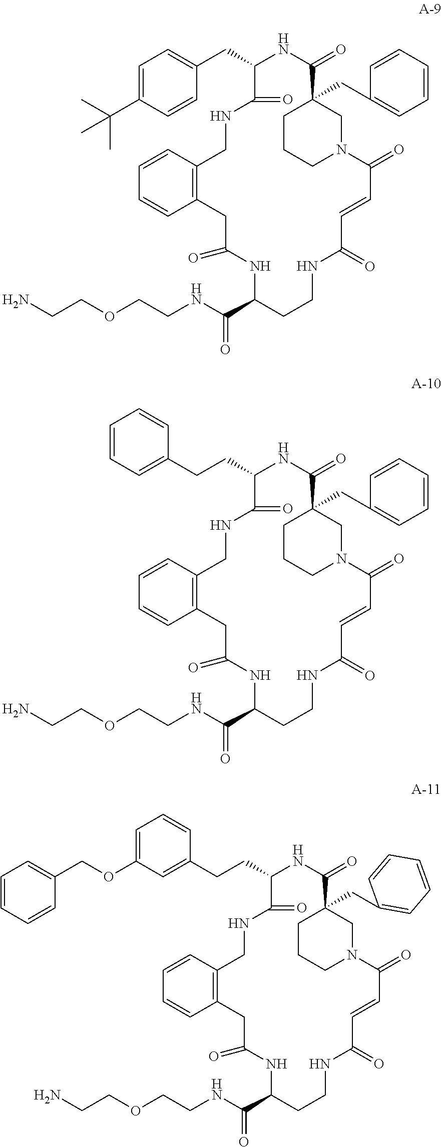 [SMILES: CC(C)(C)c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](CCc2ccccc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](CCc2cccc(OCc3ccccc3)c2)C(=O)NCc2ccccc2CC(=O)N1] 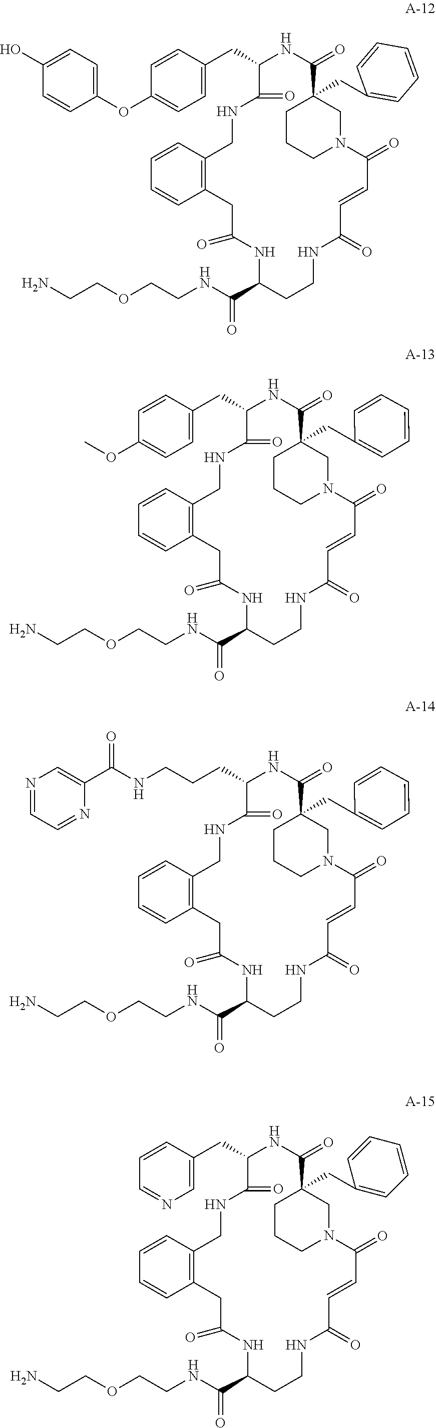 [SMILES: NCCOCCNC(=O)[C@@H]1CCNC(=O)/C=C/C(=O)N2CCC[C@](Cc3ccccc3)(C2)C(=O)N[C@@H](Cc2ccc(Oc3ccc(O)cc3)cc2)C(=O)NCc2ccccc2CC(=O)N1 ; COc1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](CCCNC(=O)c2cnccn2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccnc2)C(=O)NCc2ccccc2CC(=O)N1] 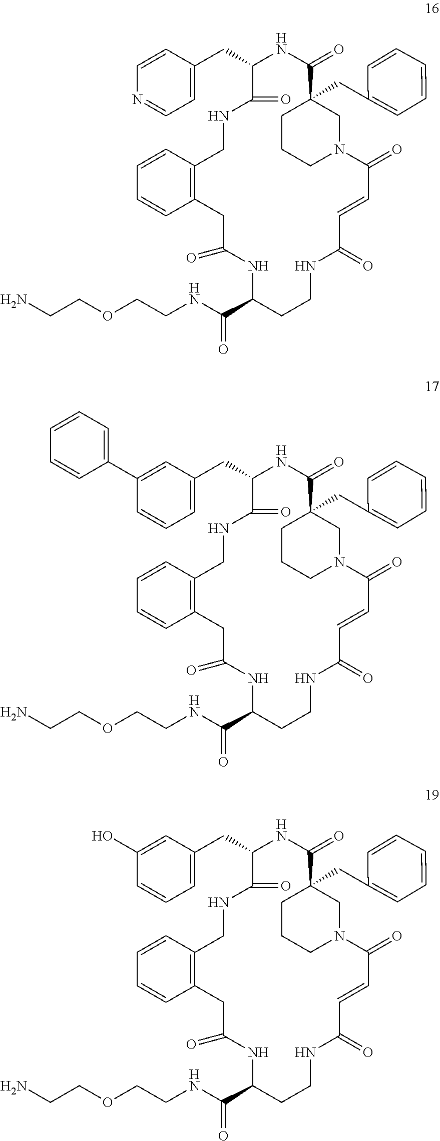 [SMILES: NCCOCCNC(=O)[C@@H]1CCNC(=O)/C=C/C(=O)N2CCC[C@](Cc3ccccc3)(C2)C(=O)N[C@@H](Cc2ccncc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccc(-c3ccccc3)c2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2cccc(O)c2)C(=O)NCc2ccccc2CC(=O)N1] 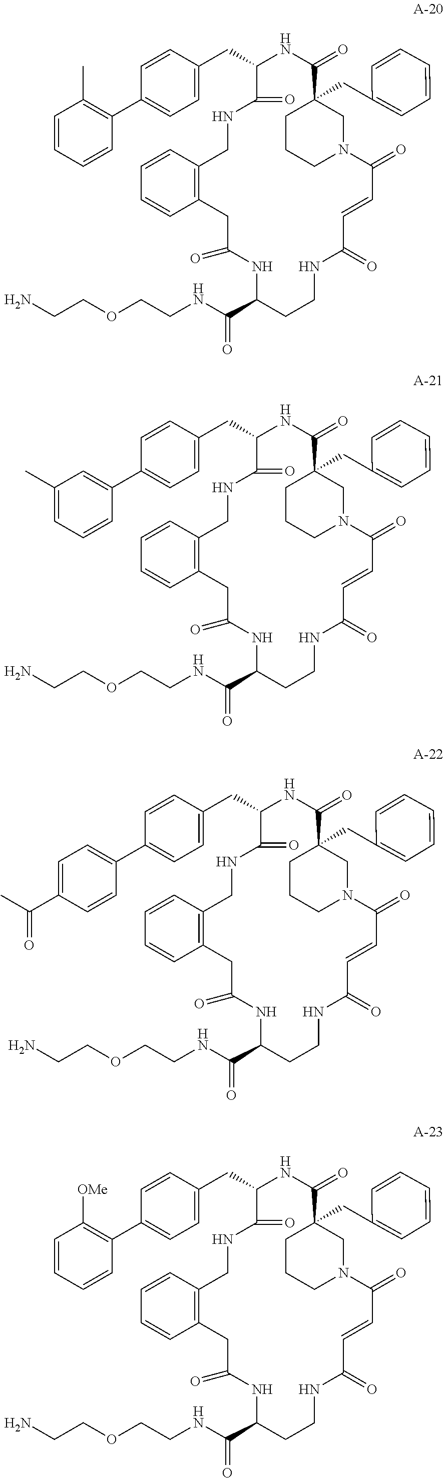 [SMILES: Cc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; Cc1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; COc1ccccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1] 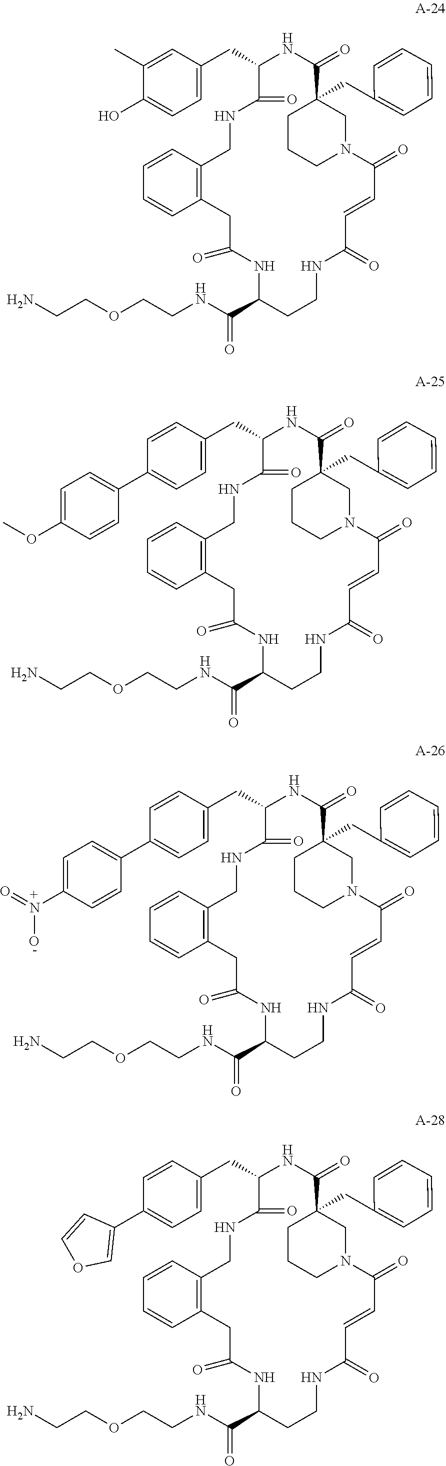 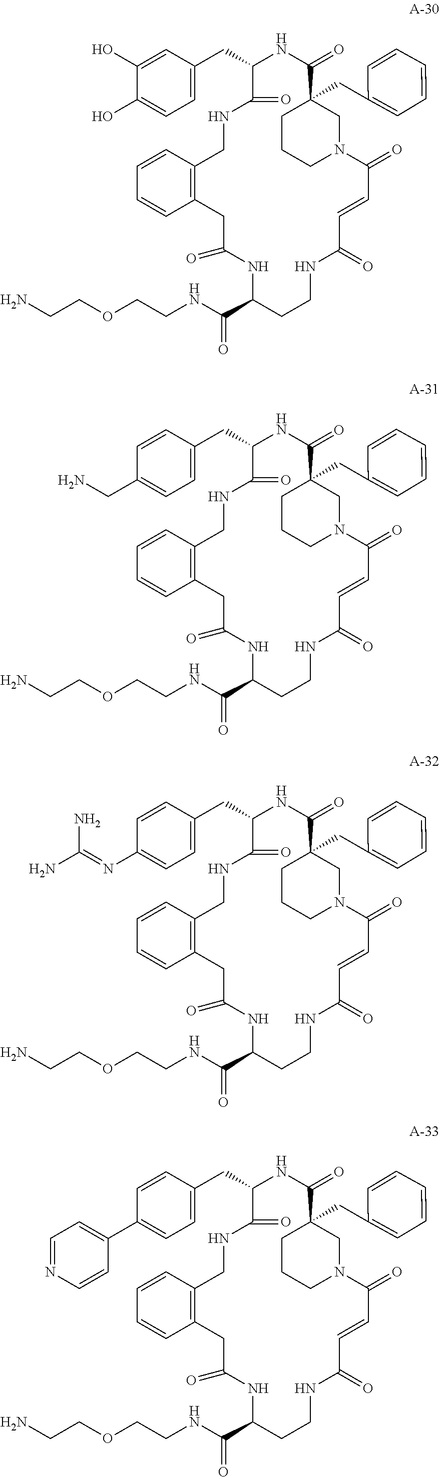 [SMILES: NCCOCCNC(=O)[C@@H]1CCNC(=O)/C=C/C(=O)N2CCC[C@](Cc3ccccc3)(C2)C(=O)N[C@@H](Cc2ccc(O)c(O)c2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(CN)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(N=C(N)N)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccncc3)cc2)C(=O)NCc2ccccc2CC(=O)N1] 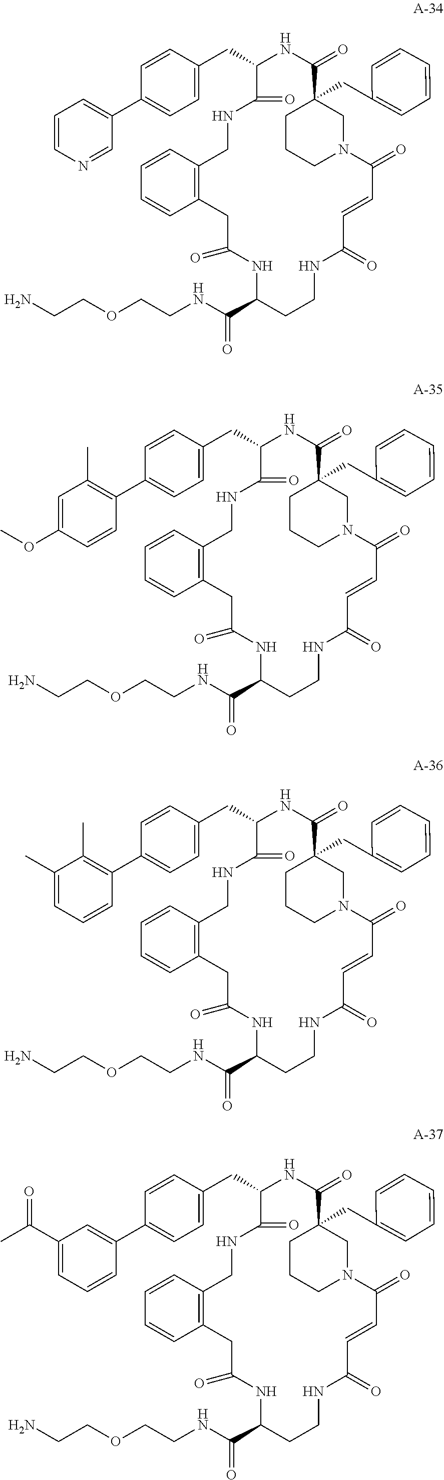 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3cccnc3)cc2)C(=O)NCc2ccccc2CC(=O)N1 ; COc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c(C)c1 ; Cc1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1C ; CC(=O)c1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1] 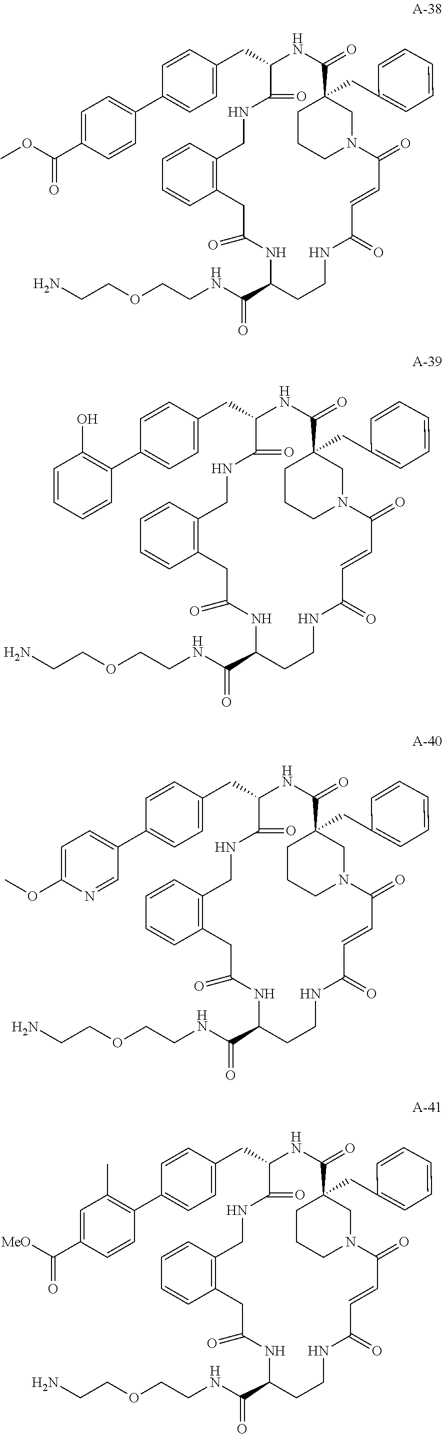 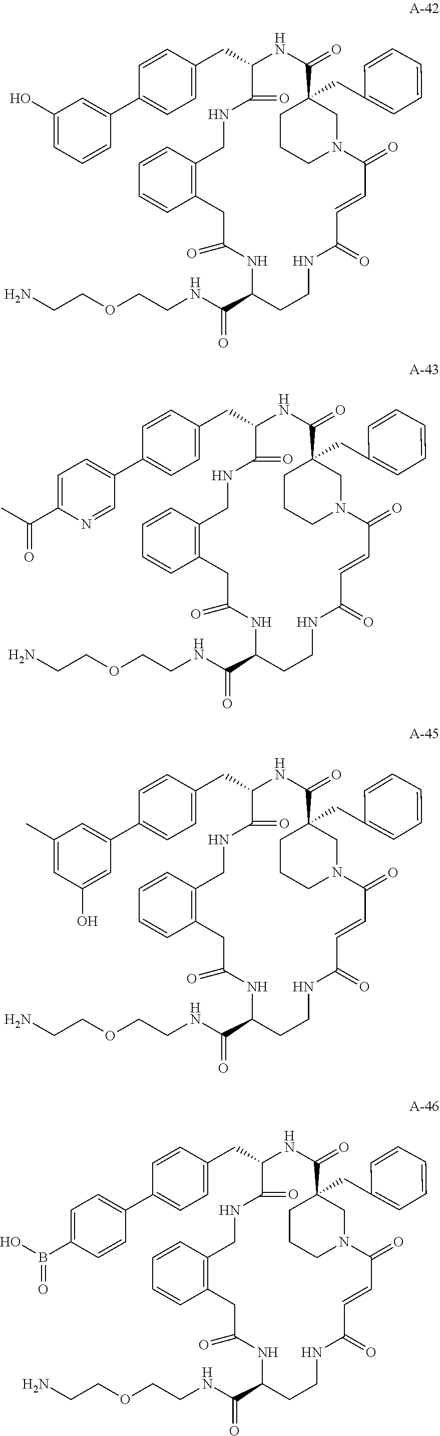 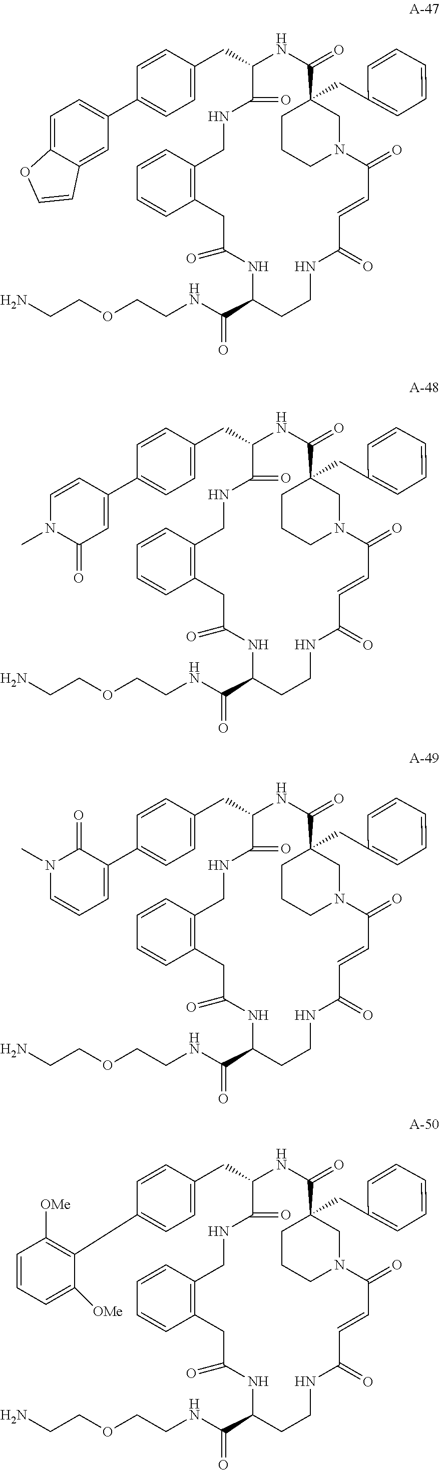 [SMILES: NCCOCCNC(=O)[C@@H]1CCNC(=O)/C=C/C(=O)N2CCC[C@](Cc3ccccc3)(C2)C(=O)N[C@@H](Cc2ccc(-c3ccc4occc4c3)cc2)C(=O)NCc2ccccc2CC(=O)N1 ; Cn1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1=O ; Cn1cccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)c1=O ; COc1cccc(OC)c1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1] 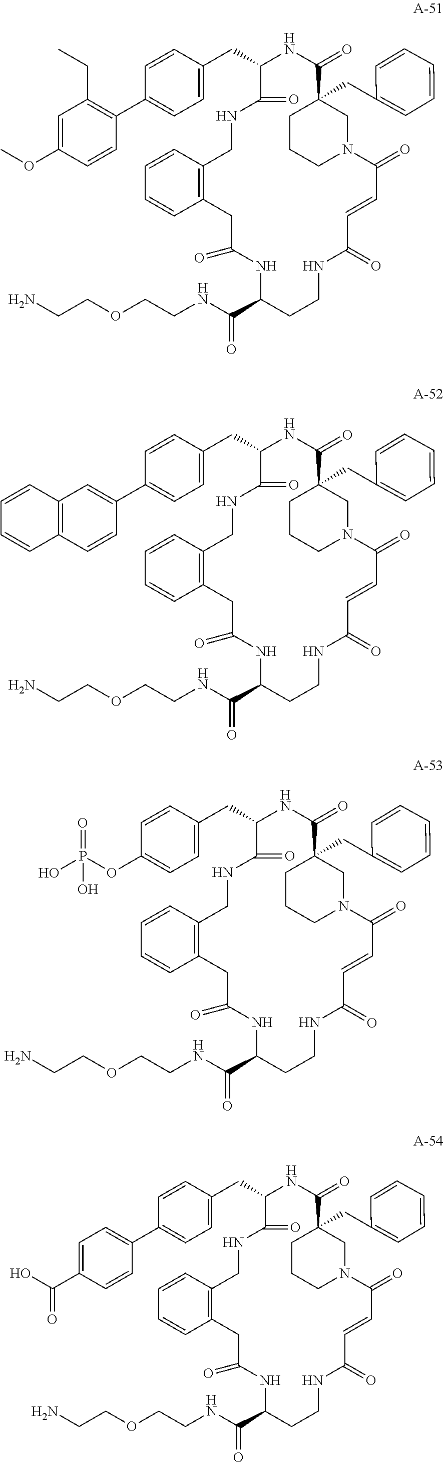 [SMILES: CCc1cc(OC)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc4ccccc4c3)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(OP(=O)(O)O)cc2)C(=O)NCc2ccccc2CC(=O)N1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(C(=O)O)cc3)cc2)C(=O)NCc2ccccc2CC(=O)N1] 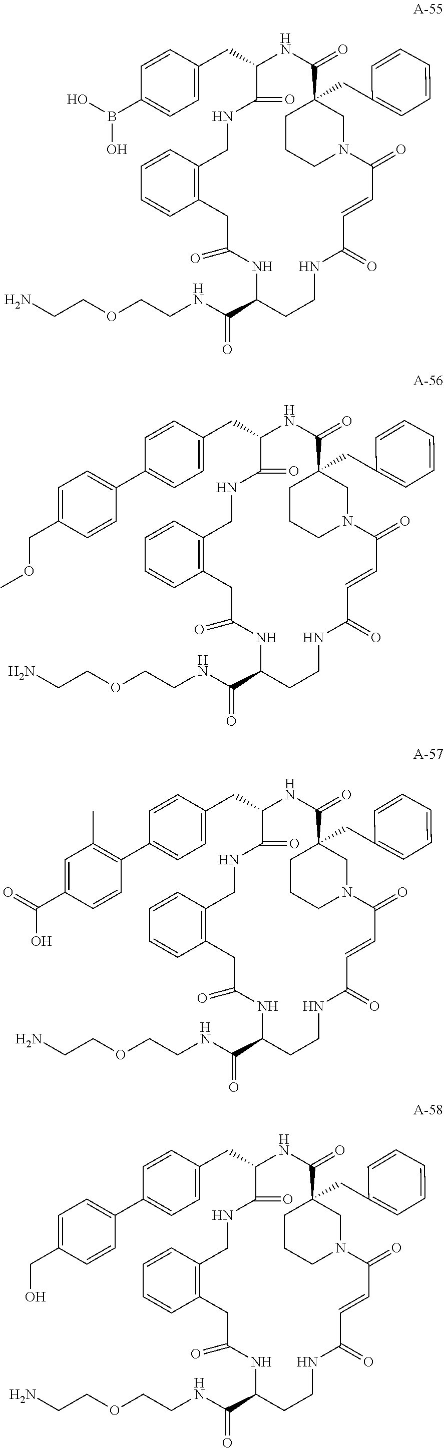 [SMILES: NCCOCCNC(=O)[C@@H]1CCNC(=O)/C=C/C(=O)N2CCC[C@](Cc3ccccc3)(C2)C(=O)N[C@@H](Cc2ccc(B(O)O)cc2)C(=O)NCc2ccccc2CC(=O)N1 ; COCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; Cc1cc(C(=O)O)ccc1-c1ccc(C[C@@H]2NC(=O)[C@]3(Cc4ccccc4)CCCN(C3)C(=O)/C=C/C(=O)NCC[C@@H](C(=O)NCCOCCN)NC(=O)Cc3ccccc3CNC2=O)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CO)cc3)cc2)C(=O)NCc2ccccc2CC(=O)N1] 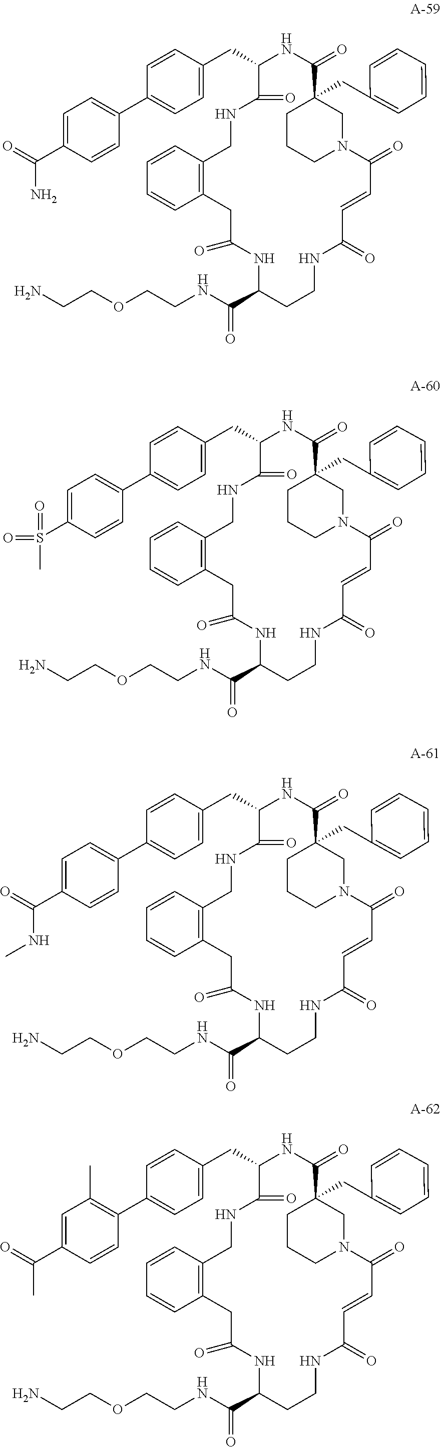 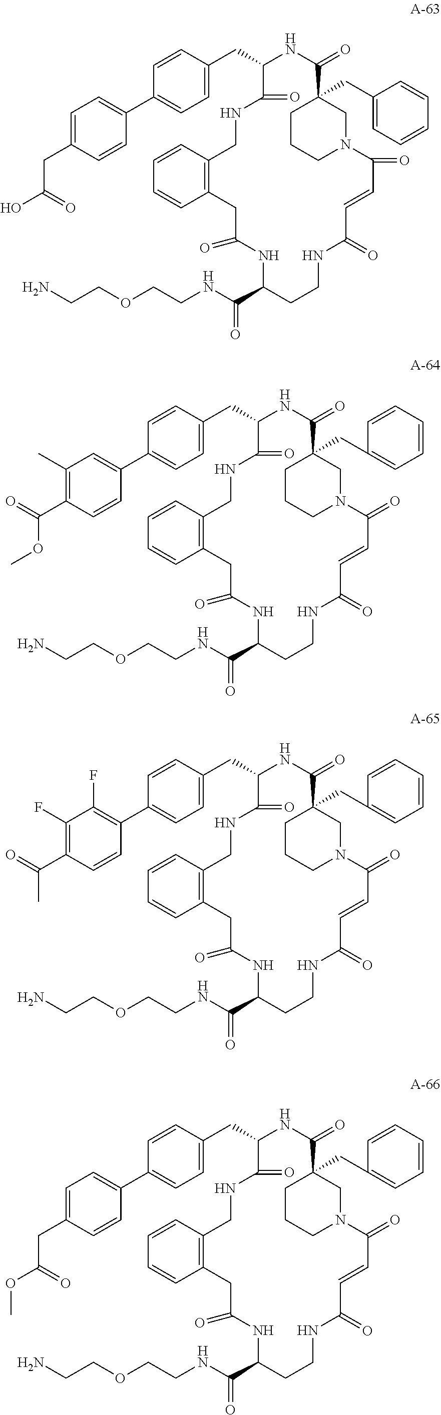 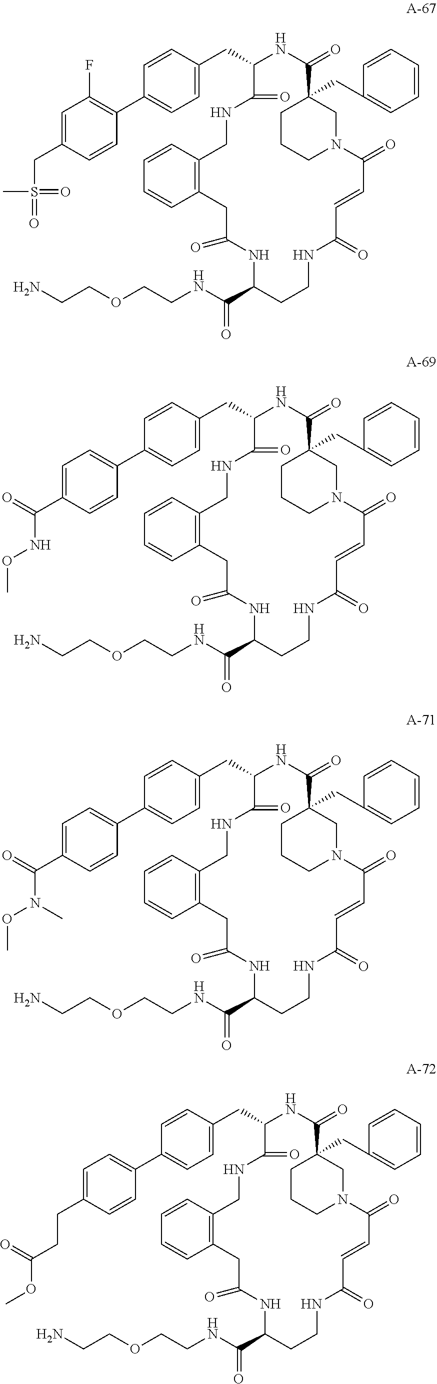 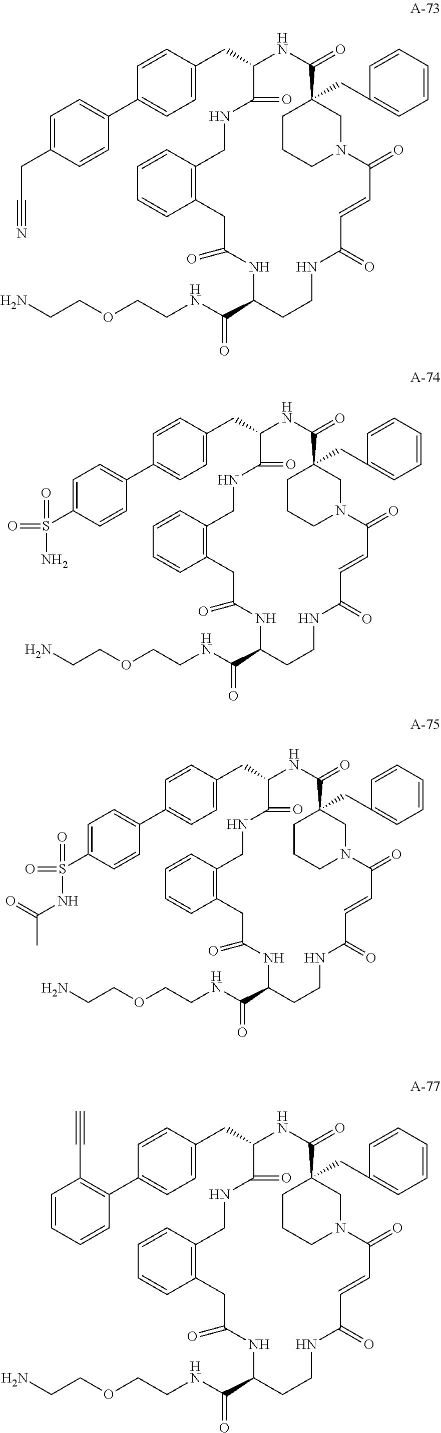 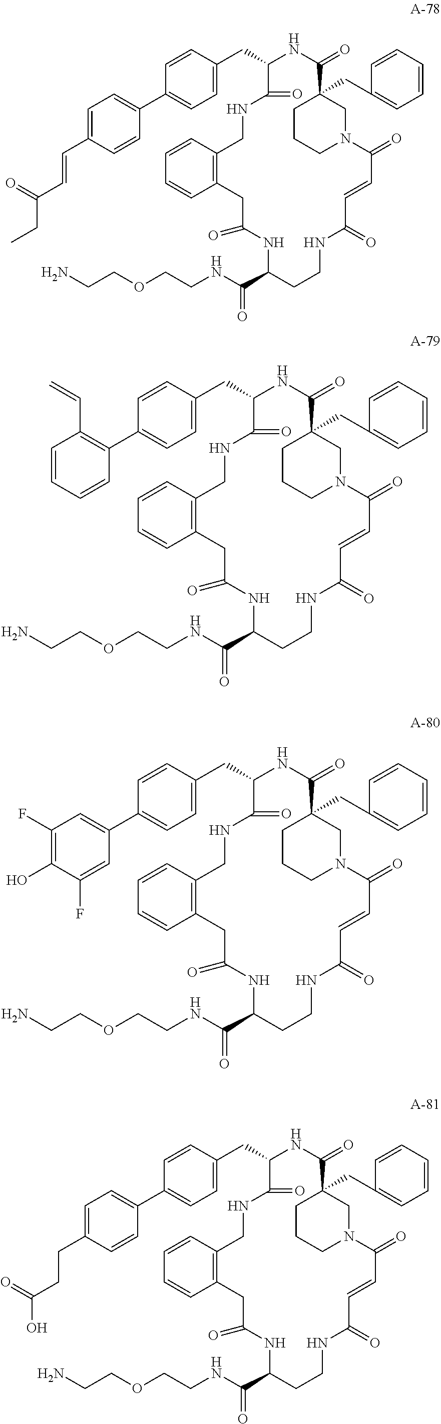 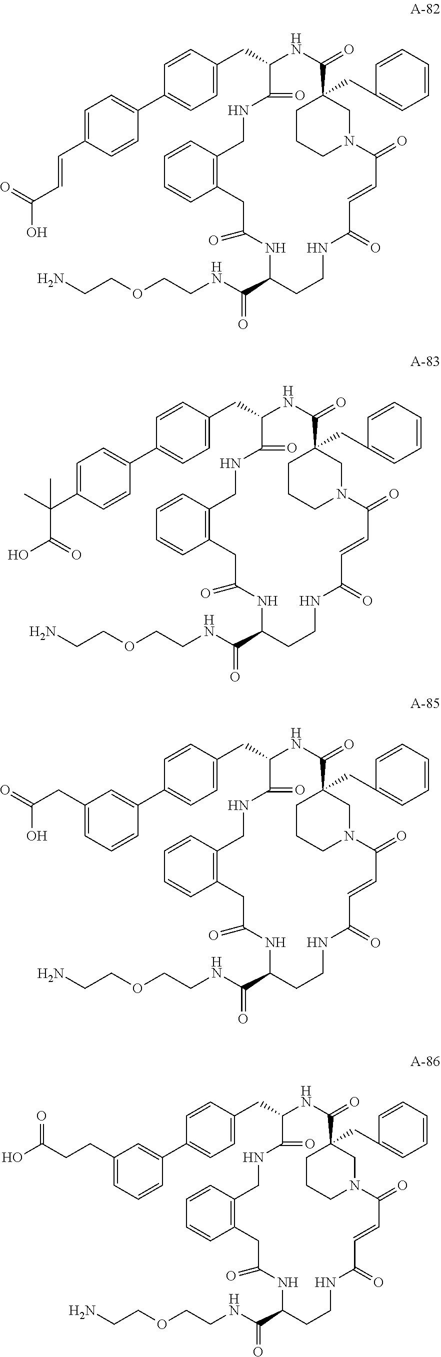 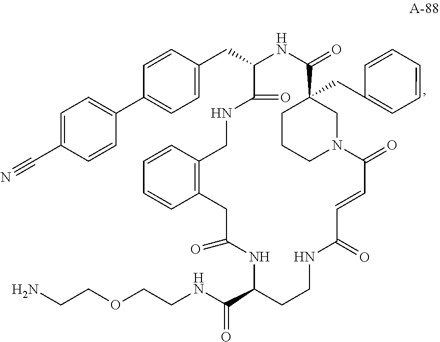 [SMILES: N#Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1]
or a pharmaceutically acceptable salt, solvate, hydrate, stereoisomer, polymorph, tautomer, isotopically enriched form, or prodrug thereof.

46. The compound of any one of claims 1-44, wherein the compound is of formula: 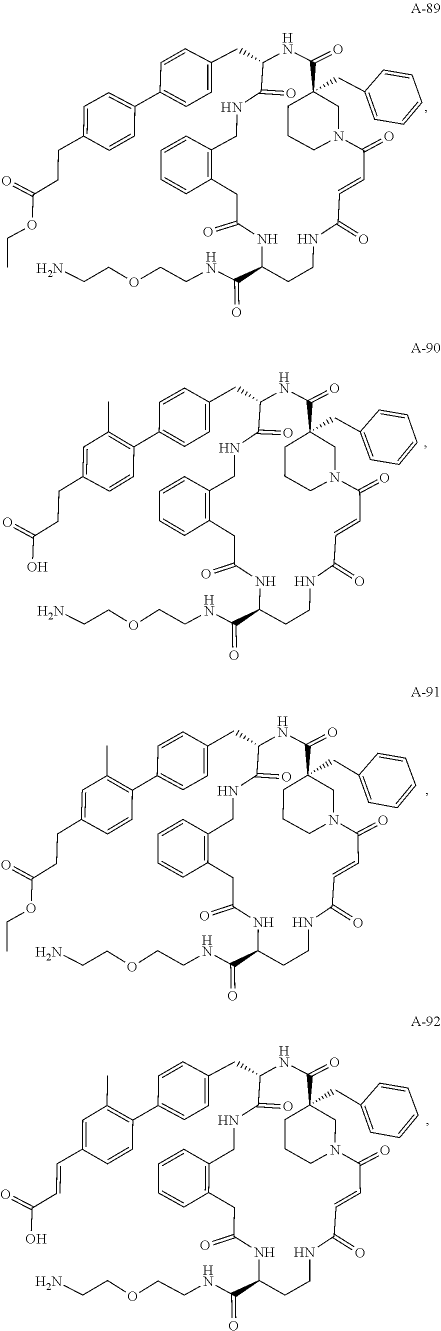 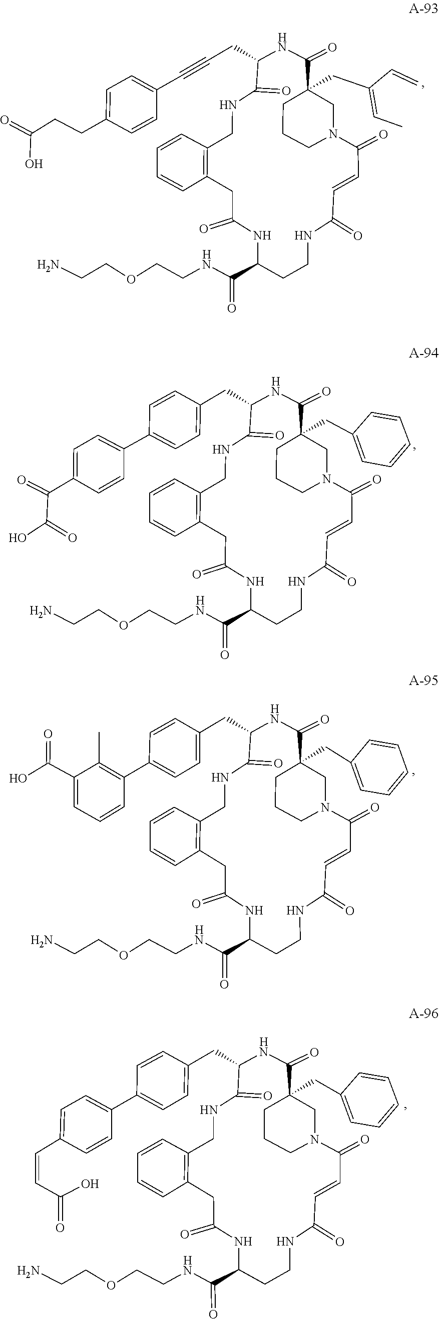 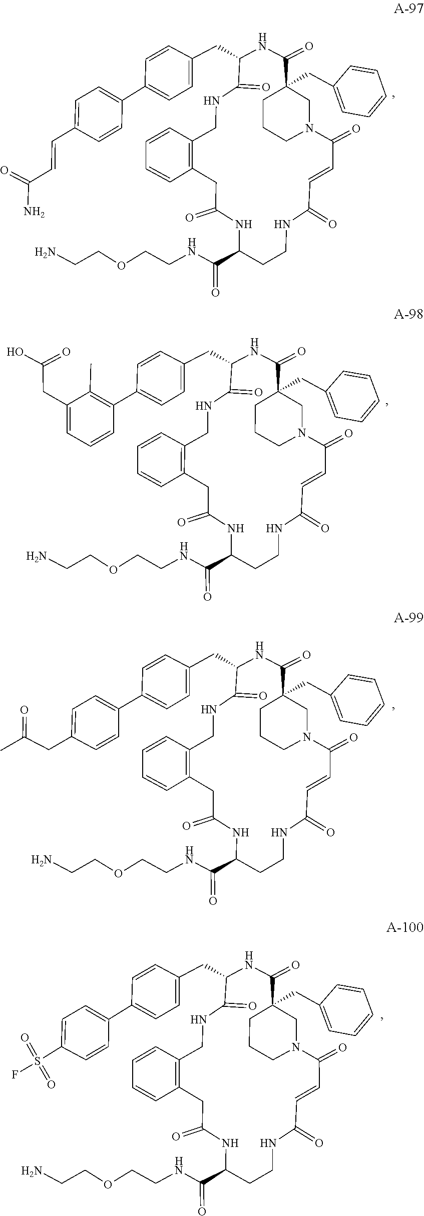 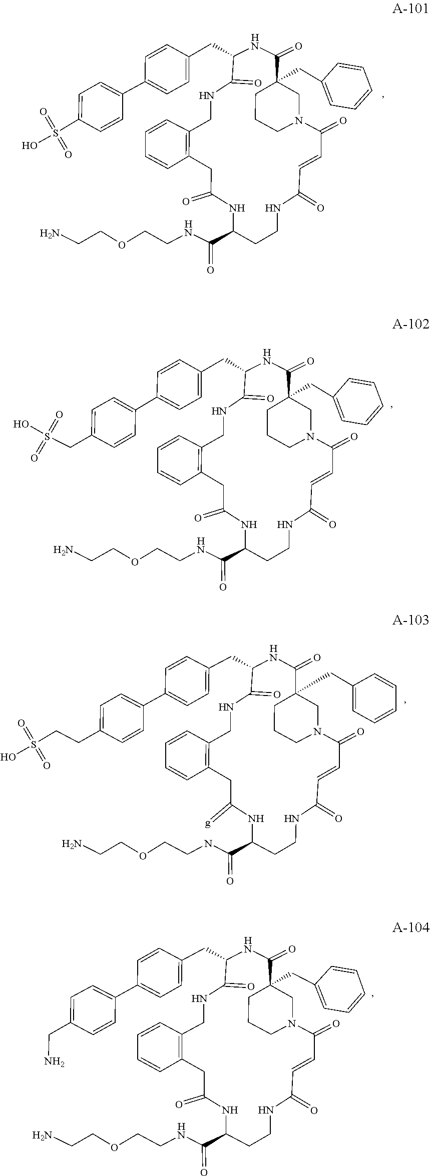 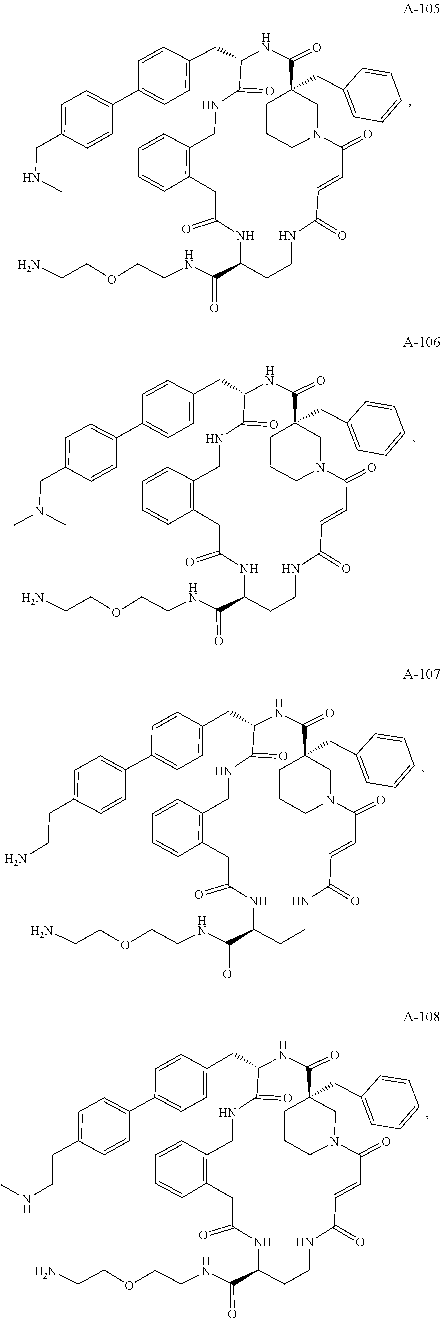 [SMILES: CNCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; CN(C)Cc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1 ; NCCOCCNC(=O)[C@@H]1CCNC(=O)/C=C/C(=O)N2CCC[C@](Cc3ccccc3)(C2)C(=O)N[C@@H](Cc2ccc(-c3ccc(CCN)cc3)cc2)C(=O)NCc2ccccc2CC(=O)N1 ; CNCCc1ccc(-c2ccc(C[C@@H]3NC(=O)[C@]4(Cc5ccccc5)CCCN(C4)C(=O)/C=C/C(=O)NCC[C@@H](C(=O)NCCOCCN)NC(=O)Cc4ccccc4CNC3=O)cc2)cc1] 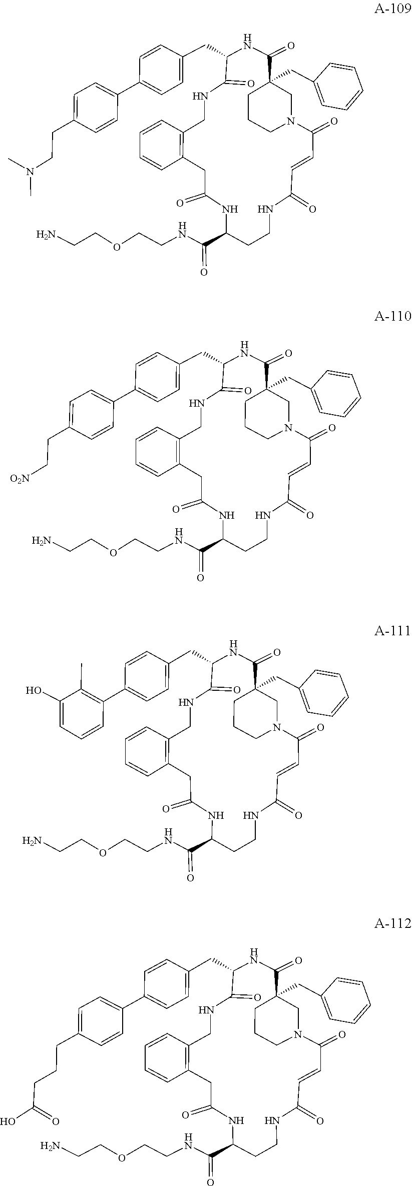 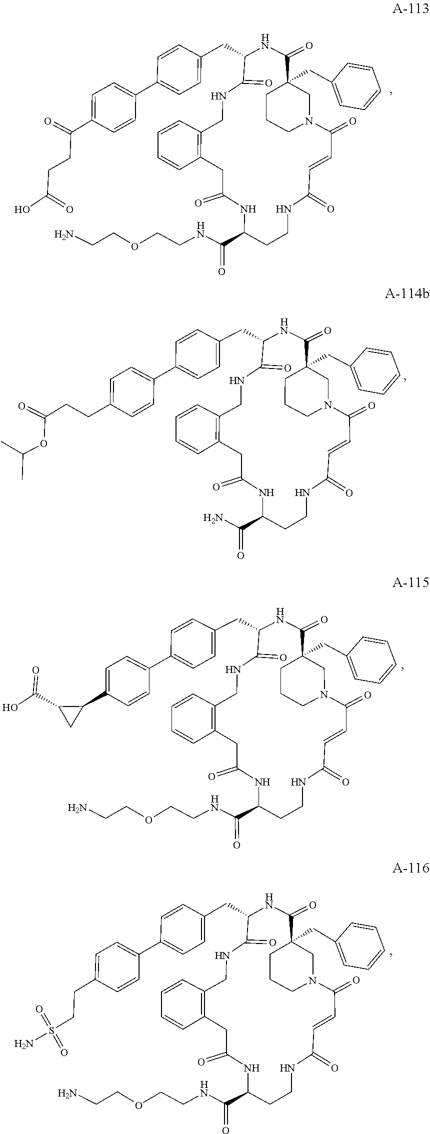 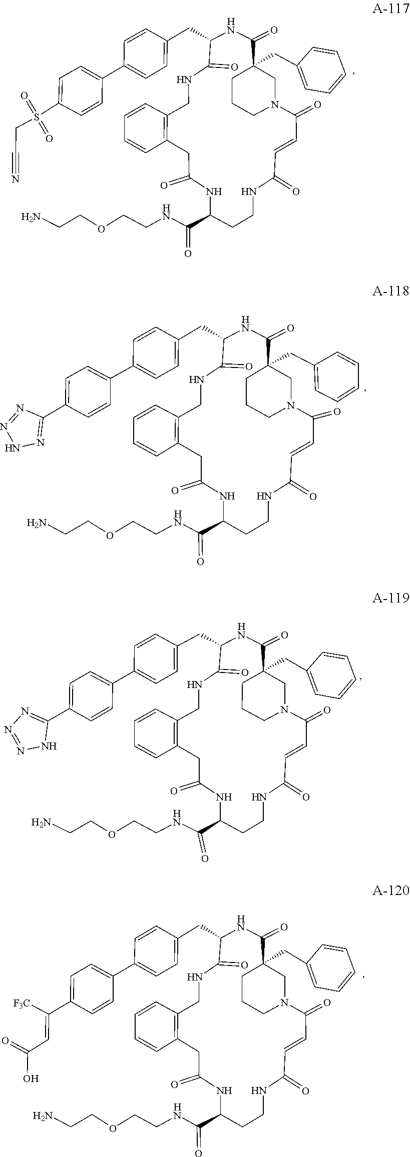 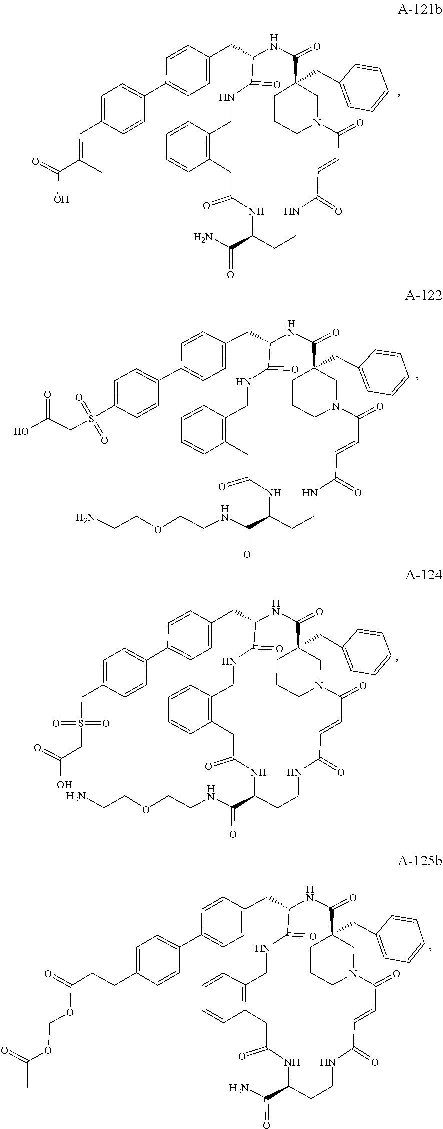 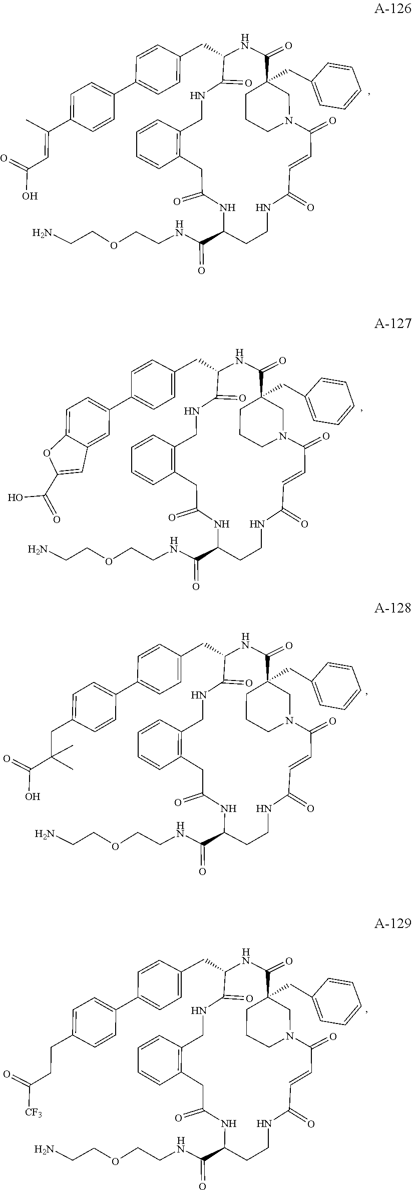 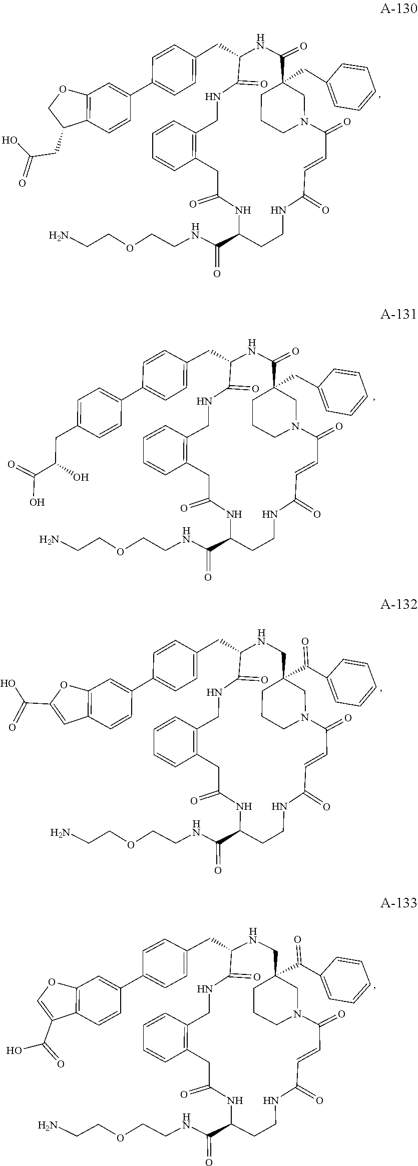 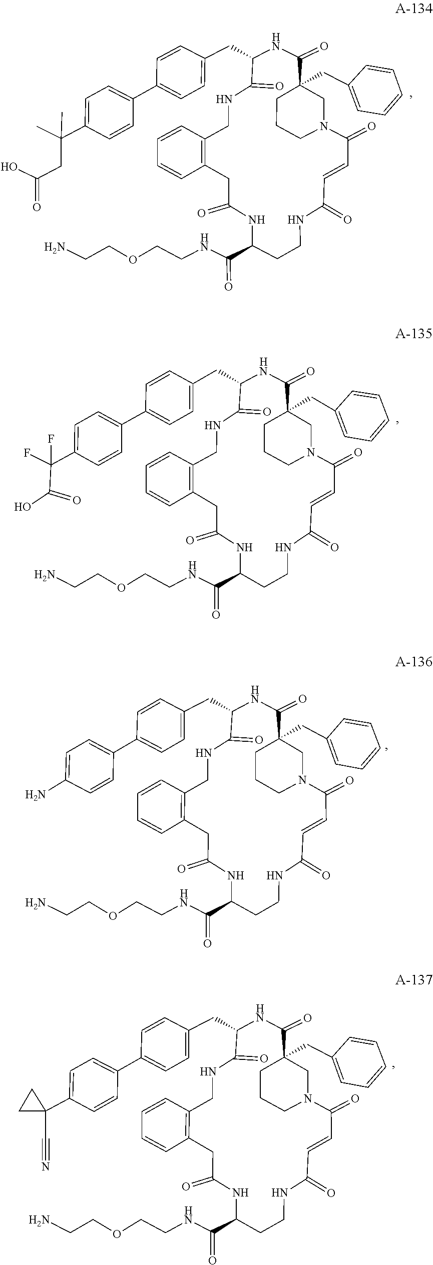 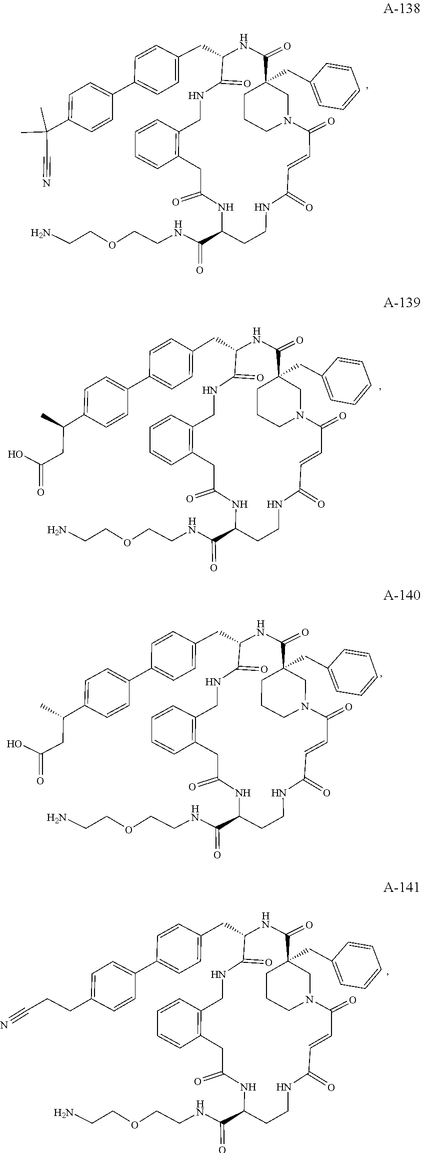 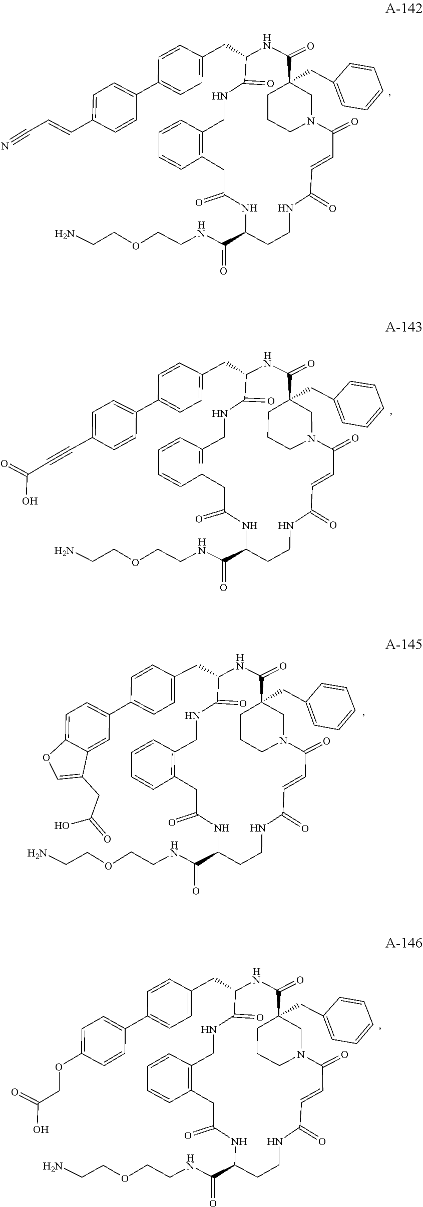 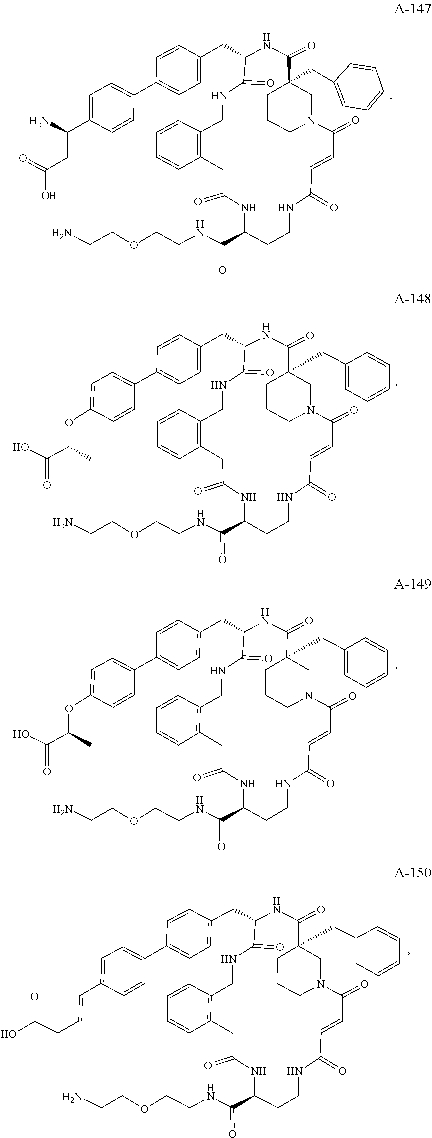 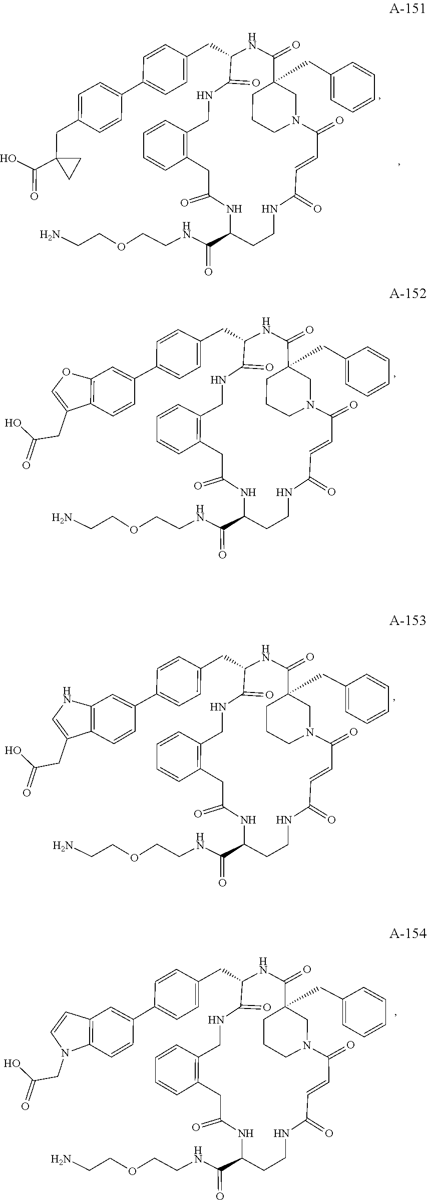 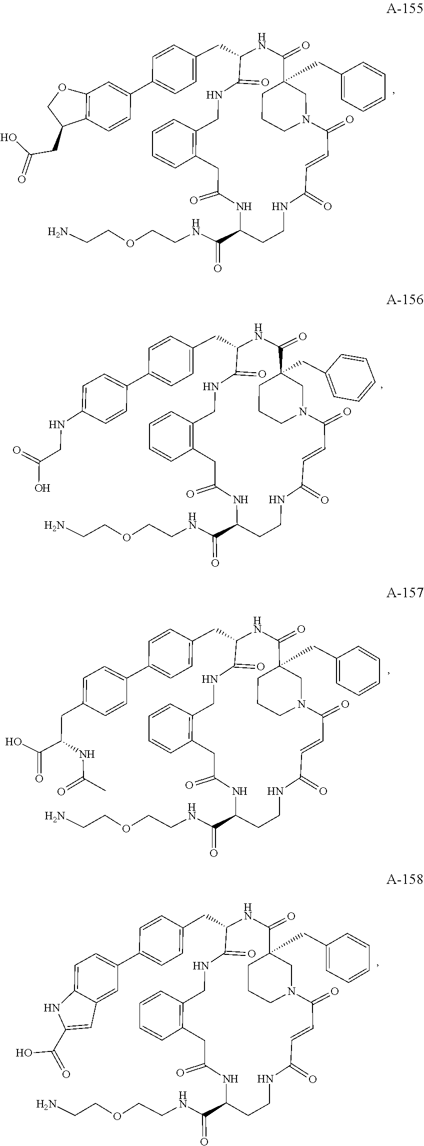 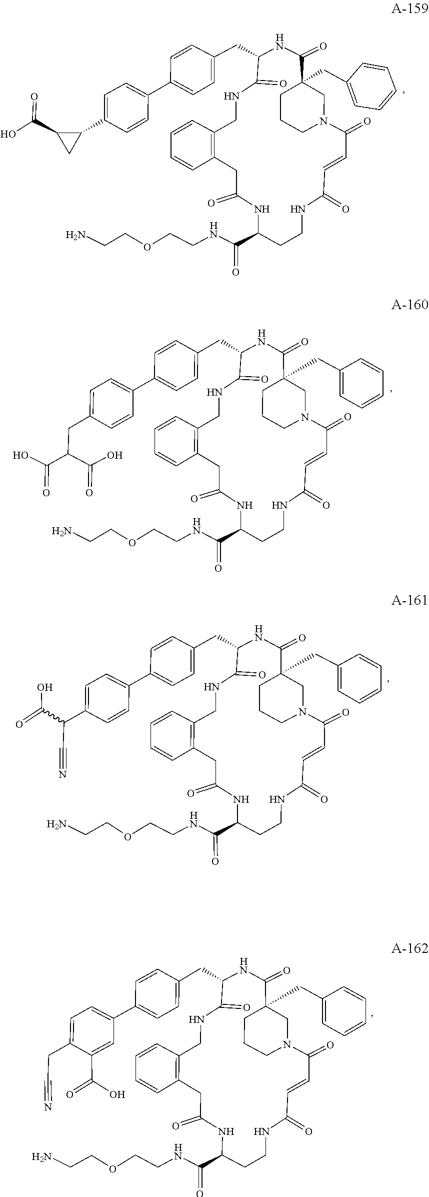 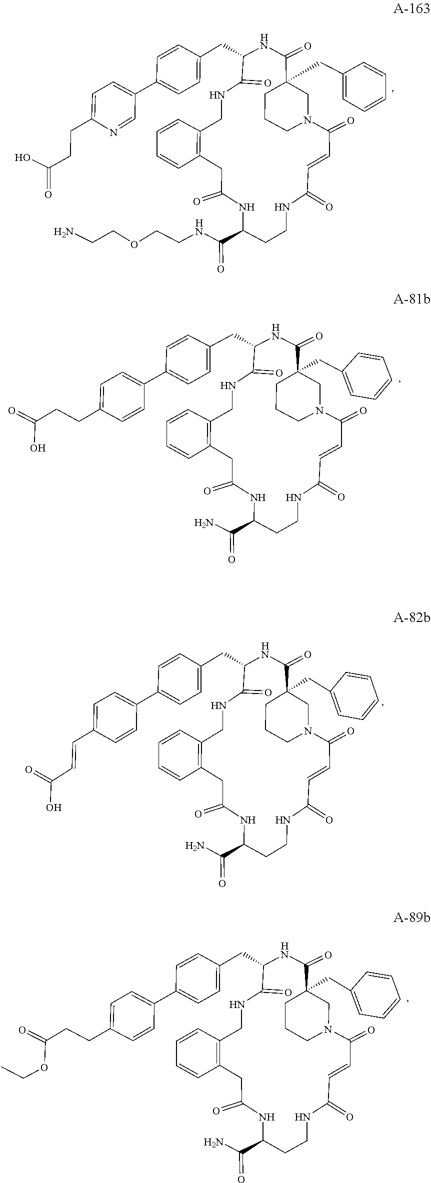 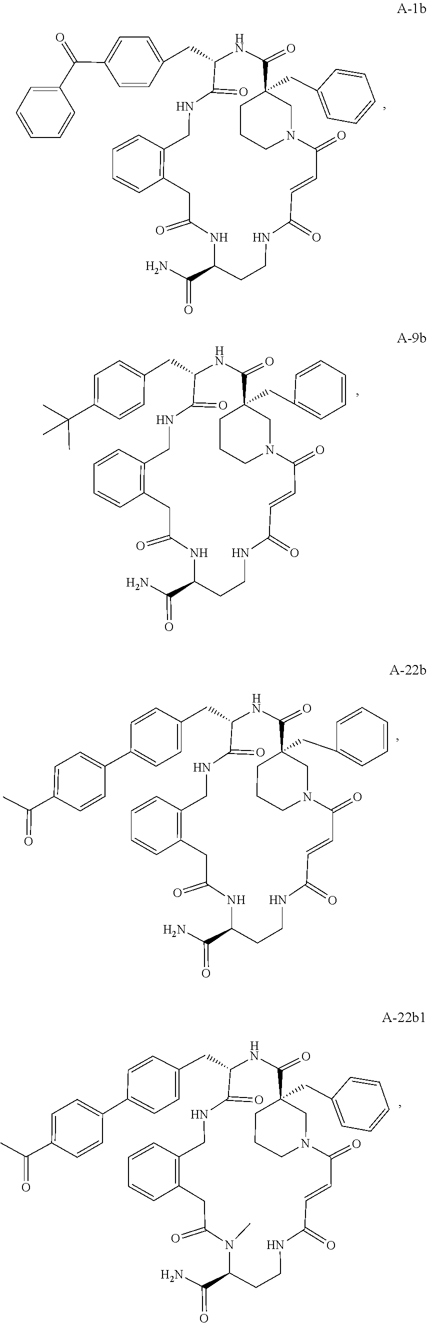 [SMILES: NC(=O)[C@@H]1CCNC(=O)/C=C/C(=O)N2CCC[C@](Cc3ccccc3)(C2)C(=O)N[C@@H](Cc2ccc(C(=O)c3ccccc3)cc2)C(=O)NCc2ccccc2CC(=O)N1 ; CC(C)(C)c1ccc(C[C@@H]2NC(=O)[C@]3(Cc4ccccc4)CCCN(C3)C(=O)/C=C/C(=O)NCC[C@@H](C(N)=O)NC(=O)Cc3ccccc3CNC2=O)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(N)=O)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(N)=O)N(C)C(=O)Cc4ccccc4CNC3=O)cc2)cc1] 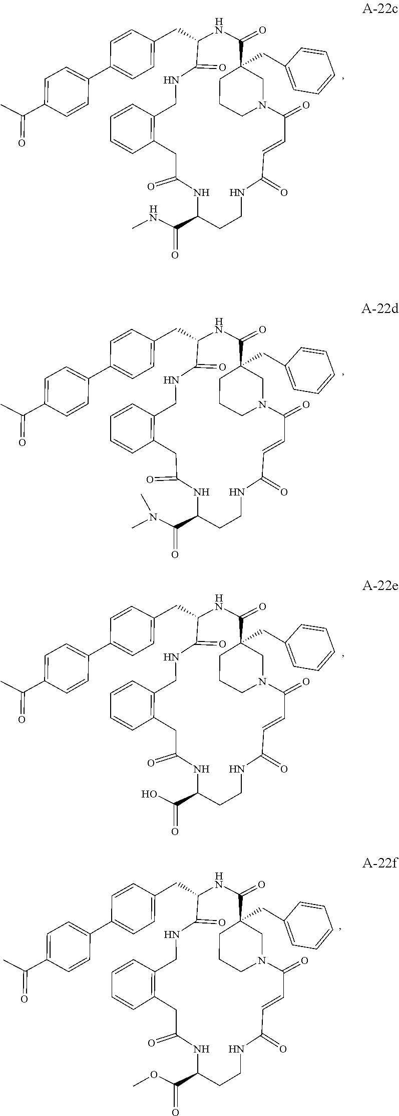 [SMILES: CNC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)N(C)C)NC(=O)Cc4ccccc4CNC3=O)cc2)cc1 ; CC(=O)c1ccc(-c2ccc(C[C@@H]3NC(=O)[C@]4(Cc5ccccc5)CCCN(C4)C(=O)/C=C/C(=O)NCC[C@@H](C(=O)O)NC(=O)Cc4ccccc4CNC3=O)cc2)cc1 ; COC(=O)[C@@H]1CCNC(=O)/C=C/C(=O)N2CCC[C@](Cc3ccccc3)(C2)C(=O)N[C@@H](Cc2ccc(-c3ccc(C(C)=O)cc3)cc2)C(=O)NCc2ccccc2CC(=O)N1] 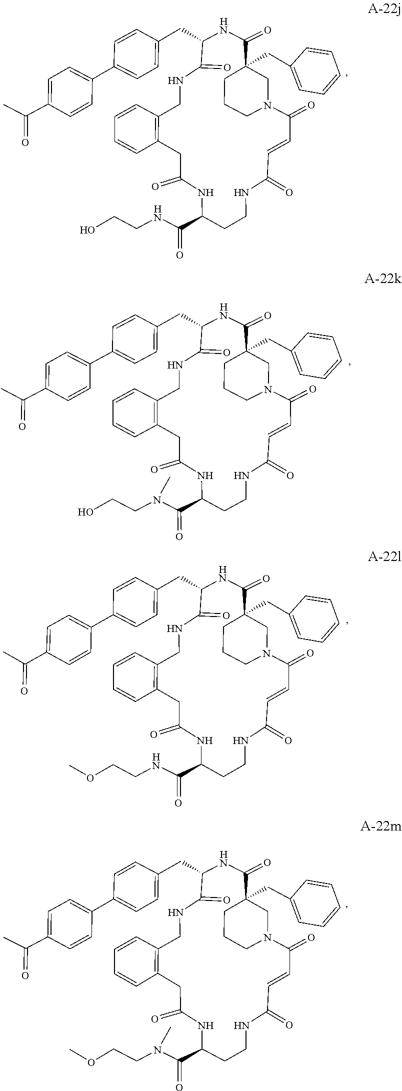 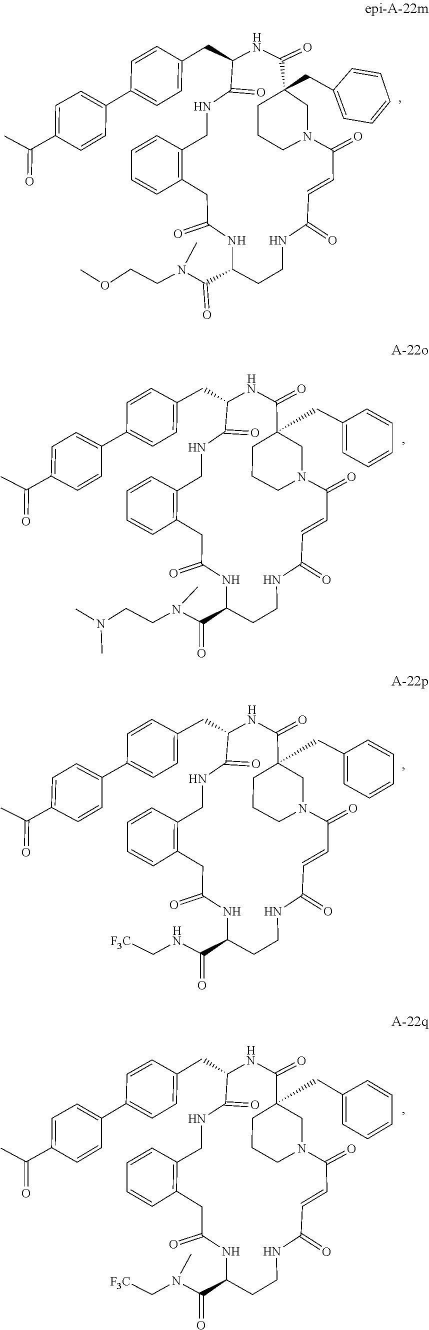
or a pharmaceutically acceptable salt, solvate, hydrate, stereoisomer, polymorph, tautomer, isotopically enriched form, or prodrug thereof.

47. A pharmaceutical composition comprising a compound of any one of claims 1-46, or a pharmaceutically acceptable salt, solvate, hydrate, polymorph, co-crystal, tautomer, stereoisomer, isotopically labeled derivative, prodrug, composition, or mixture thereof, and optionally a pharmaceutically acceptable excipient.

48. The pharmaceutical composition of claim 47, wherein the pharmaceutical composition comprises a therapeutically effective amount of the compound for use in treating a disease in a subject in need thereof.

49. A method of treating a disease and/or condition in a subject in need thereof, the method comprising administering to the subject a therapeutically effective amount of a compound of any one of claims 1-46, or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48.

50. A method of treating a disease and/or condition in a subject in need thereof, the method comprising administering to the subject a therapeutically effective amount of a compound of any one of claims 1-46, or a compound of formula: 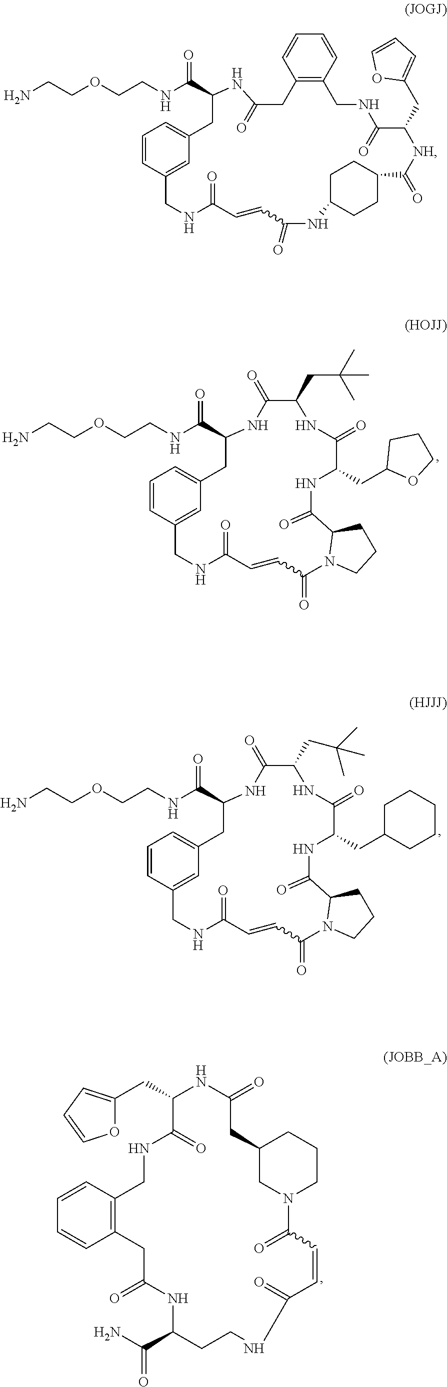 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](CC2CCCO2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2] 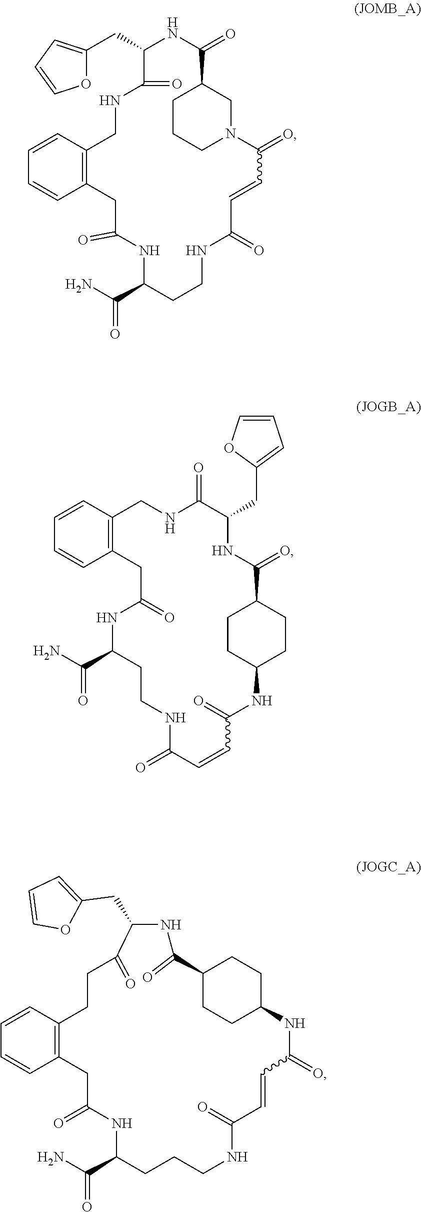 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)CCc2ccccc2CC(=O)N1] 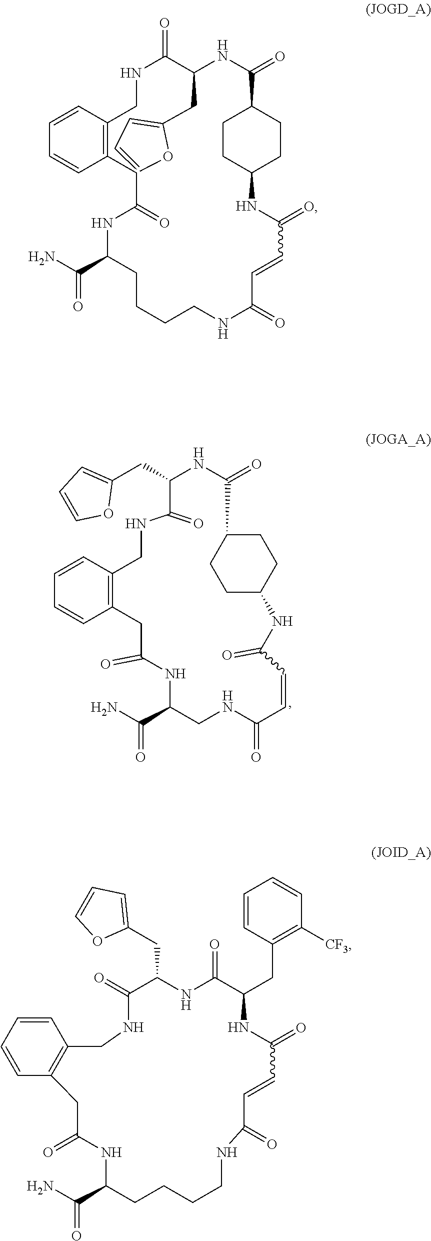 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1] 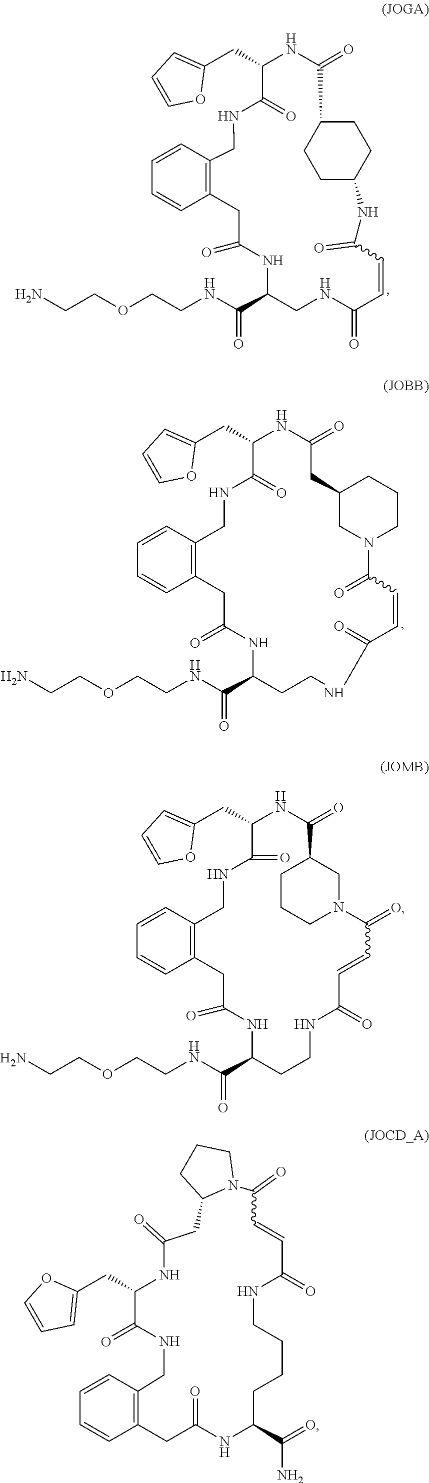 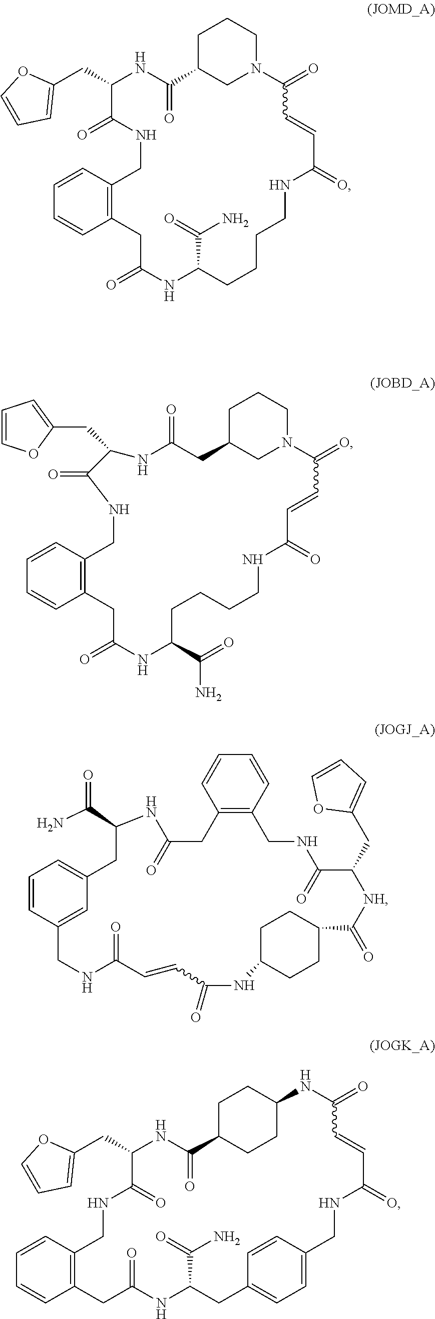 [SMILES: NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1] 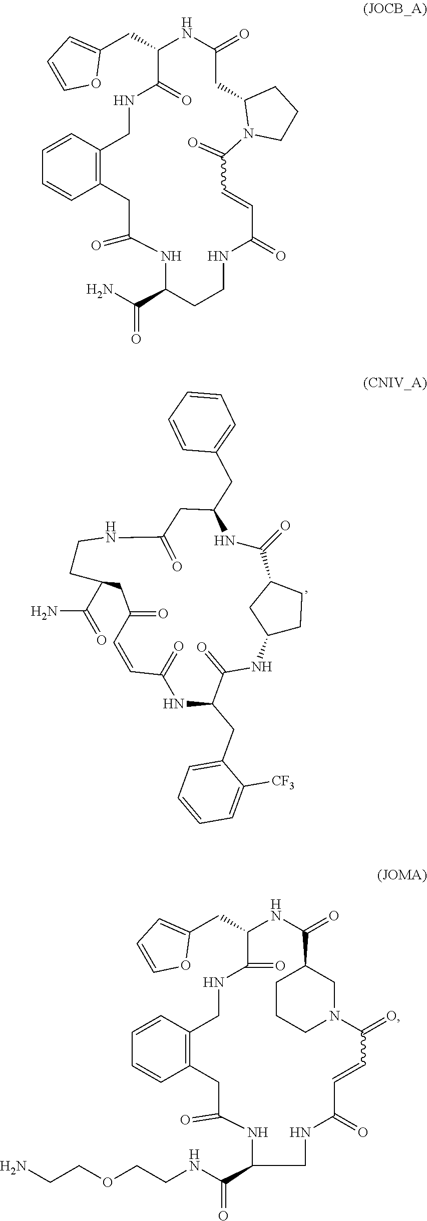 [SMILES: NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)/C=C\C(=O)C1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1] 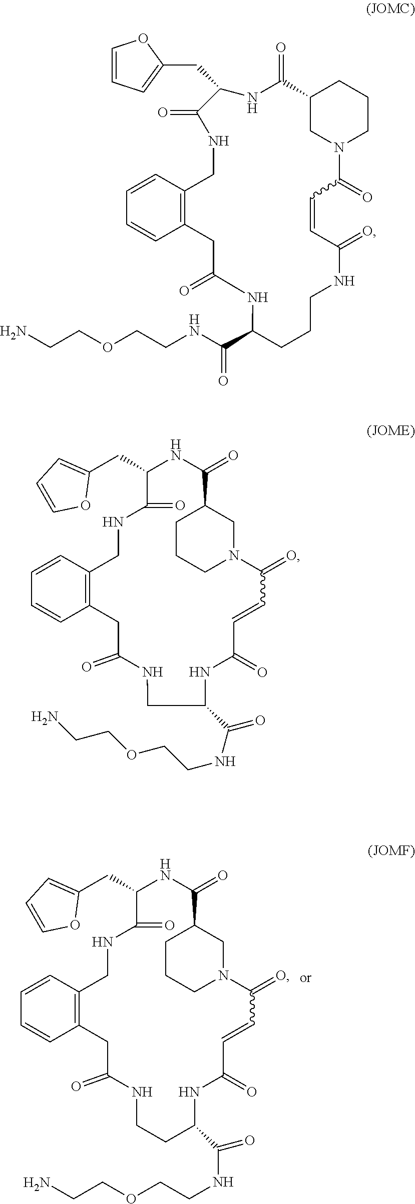 [SMILES: NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1] 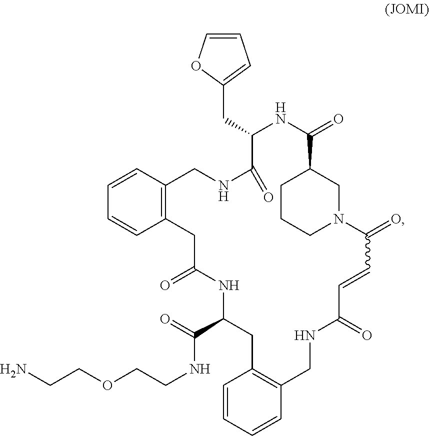 [SMILES: NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]
or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48;
provided that the disease and/or condition is not a cardiovascular disease, metabolic disorder, or a disease associated with insulin-degrading enzyme (IDE).

51. The method of claim 49 or 50, wherein the disease and/or condition is associated with the mitochondria.

52. The method of one of claims 49-51, wherein the disease and/or condition is associated with regulation of the mitochondrial permeability transition pore (mPTP).

53. The method of claim 52, wherein the disease and/or condition is associated with regulation of the opening and/or closing of the mPTP.

54. The method of any one of claims 49-53, wherein the condition is associated with autophagy.

55. The method of any one of claims 49-54, wherein the condition is associated with aging.

56. The method of claim 49 or 50, wherein the disease is a neurodegenerative disease.

57. The method of claim 49 or 50, wherein the disease is a metabolic disease.

58. The method of claim 49 or 50, wherein the disease is a proliferative disease.

59. The method of claim 58, wherein the disease is cancer.

60. The method of claim 49, 50, 58, or 59, wherein the condition is associated with oxidative stress.

61. The method of claim 50, wherein the disease and/or condition is associated with the mitochondria, the condition is associated with autophagy, the condition is associated with aging, the disease is a neurodegenerative disease, the disease is a proliferative disease, or the condition is associated with oxidative stress.

62. The method of claim 49, 50, 60, or 61, wherein the condition is ischemia-reperfusion injury.

63. A method of reducing oxidative stress in a subject in need thereof, the method comprising:
administering to the subject a therapeutically effective amount of a compound of any one of claims 1-46, or a compound of formula: 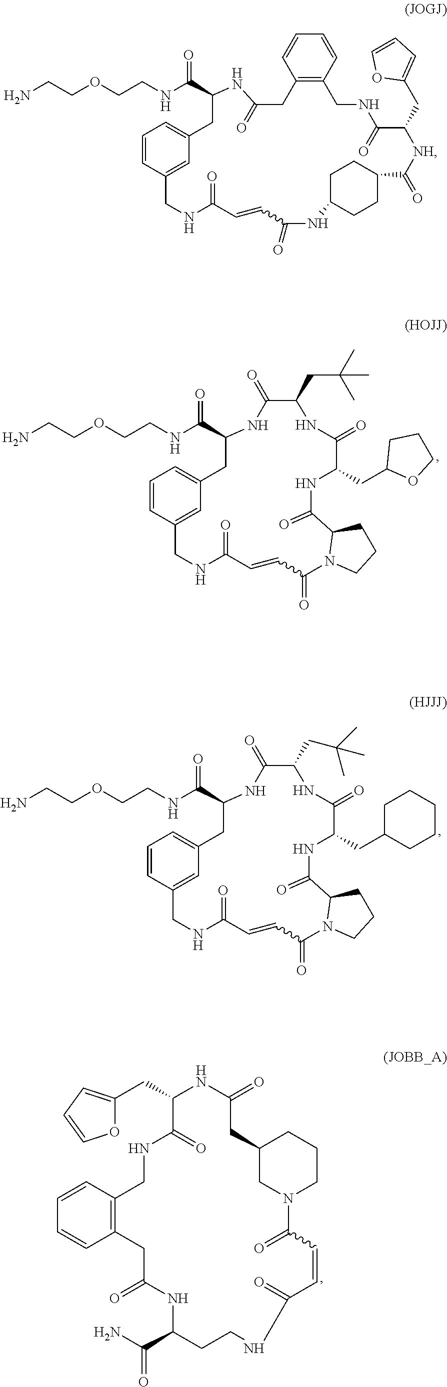 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](CC2CCCO2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2] 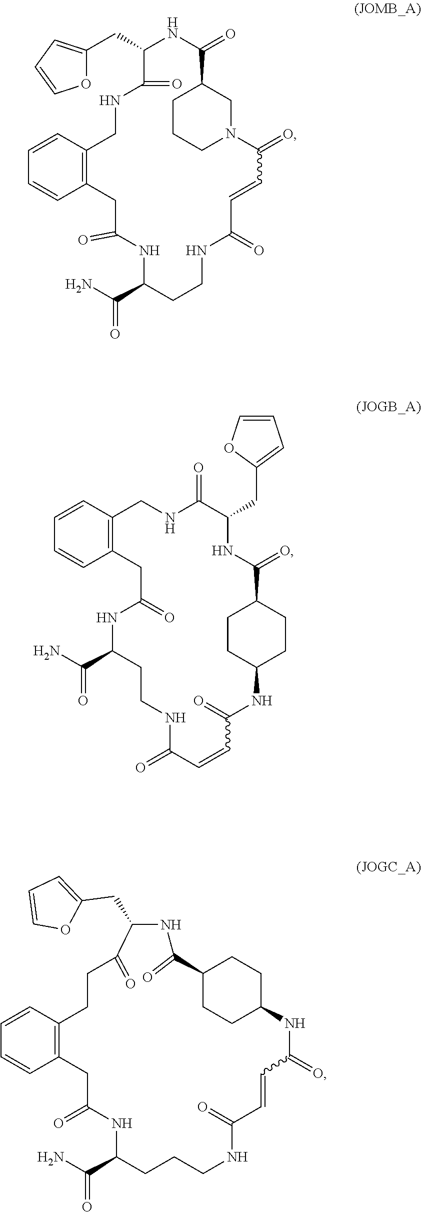 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)CCc2ccccc2CC(=O)N1] 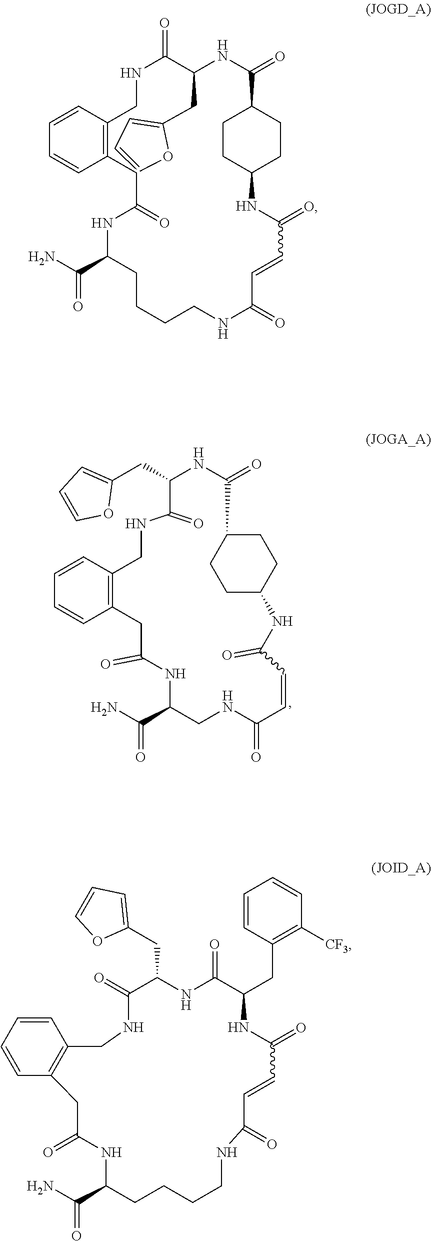 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1] 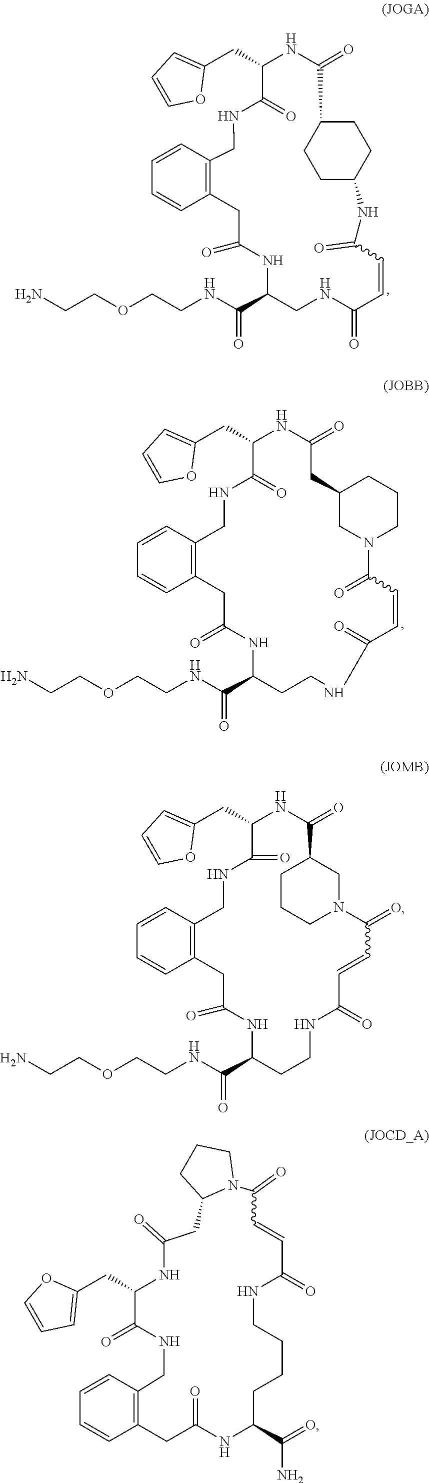 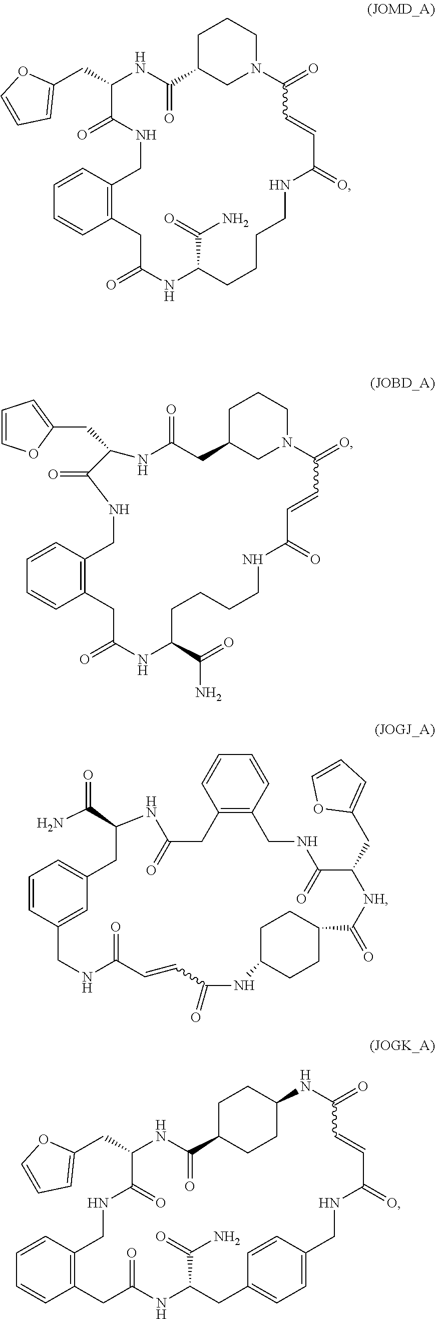 [SMILES: NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1] 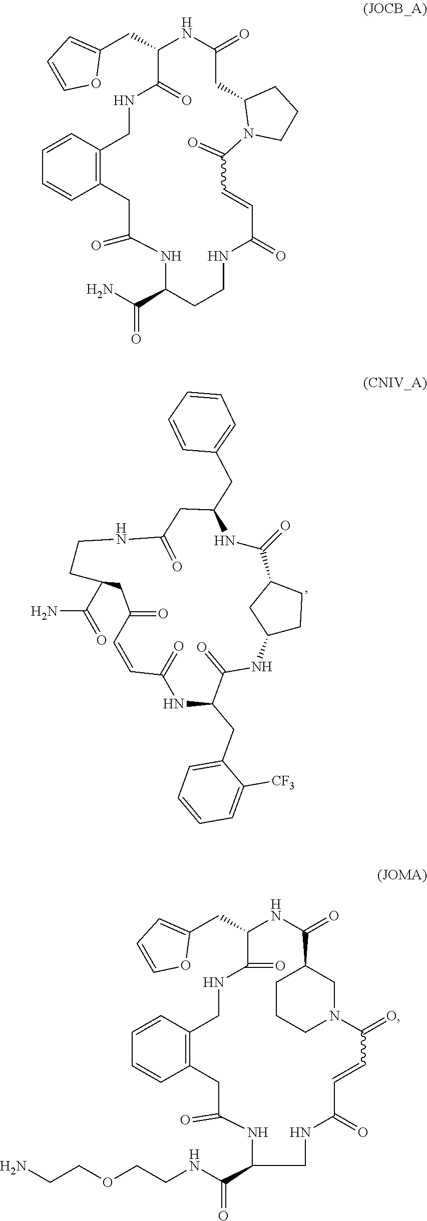 [SMILES: NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)/C=C\C(=O)C1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1] 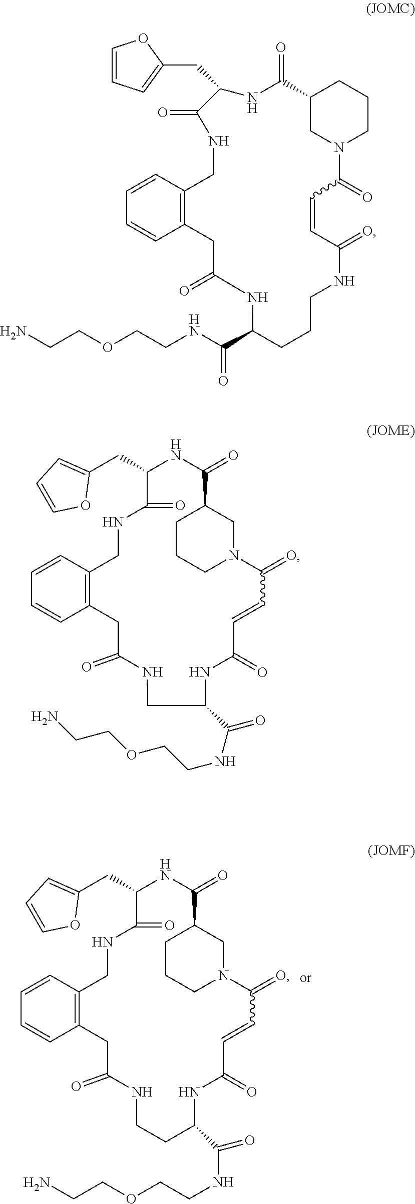 [SMILES: NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1] 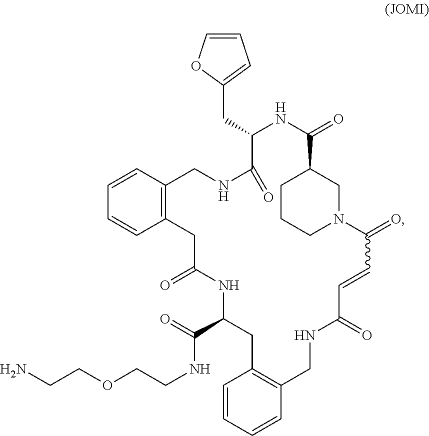 [SMILES: NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]
or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48.

64. A method of reducing oxidative stress in a biological sample, the method comprising:
contacting the biological sample with an effective amount of a compound of any one of claims 1-46, or a compound of formula: 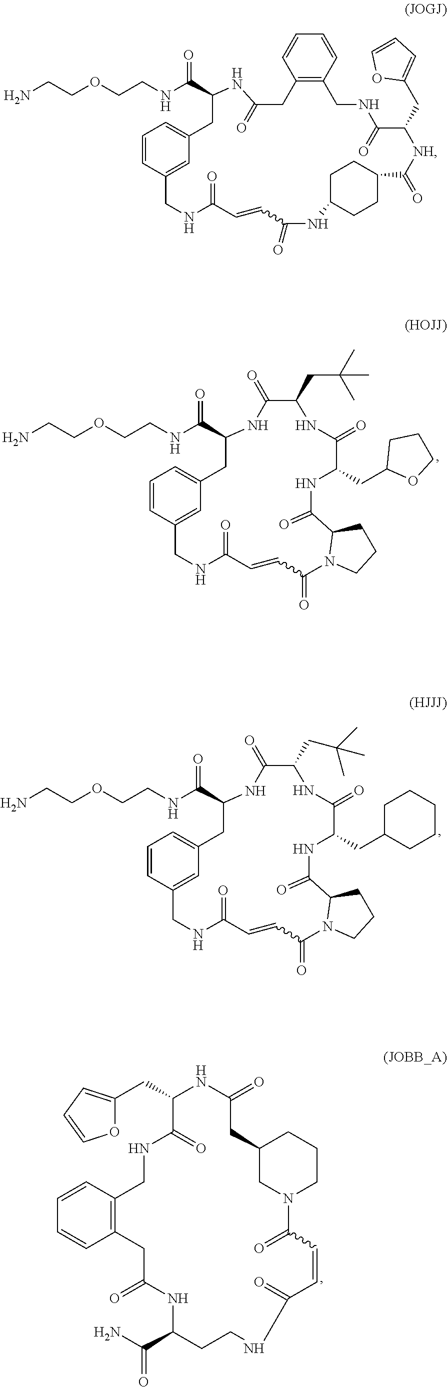 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](CC2CCCO2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2] 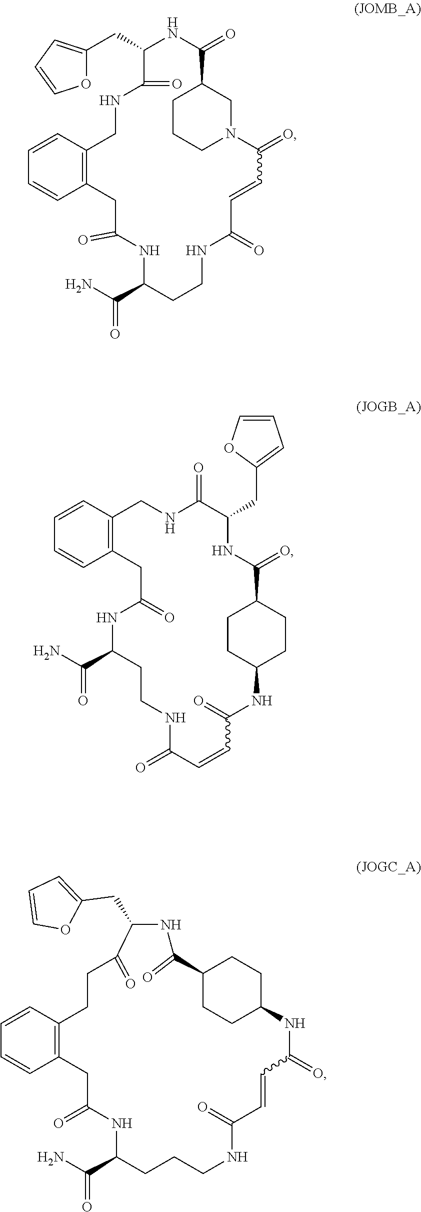 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)CCc2ccccc2CC(=O)N1] 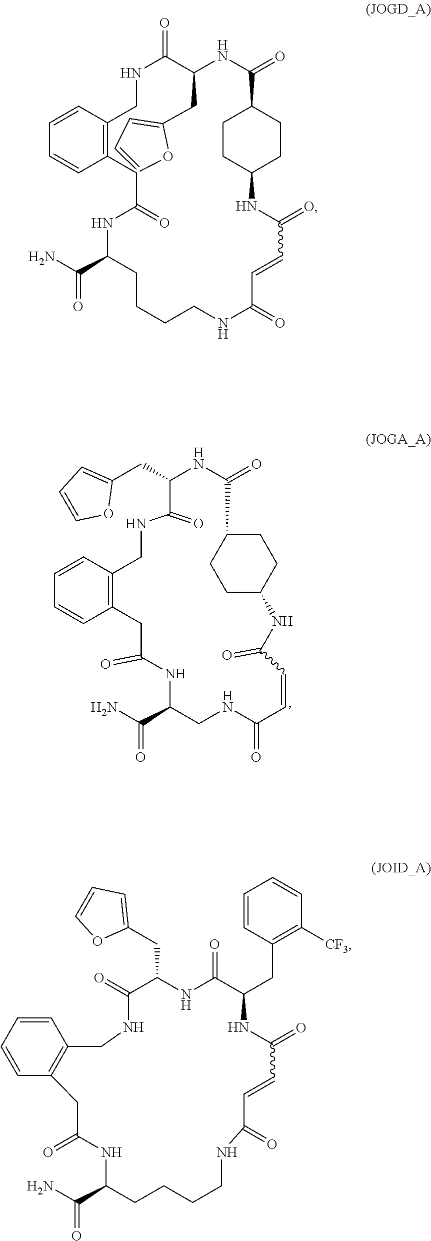 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1] 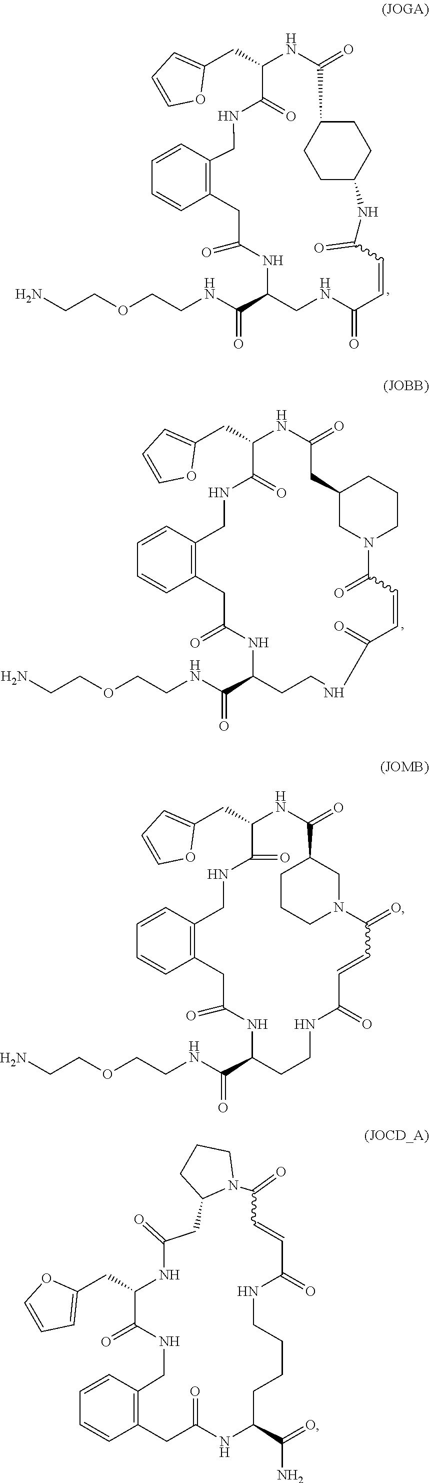 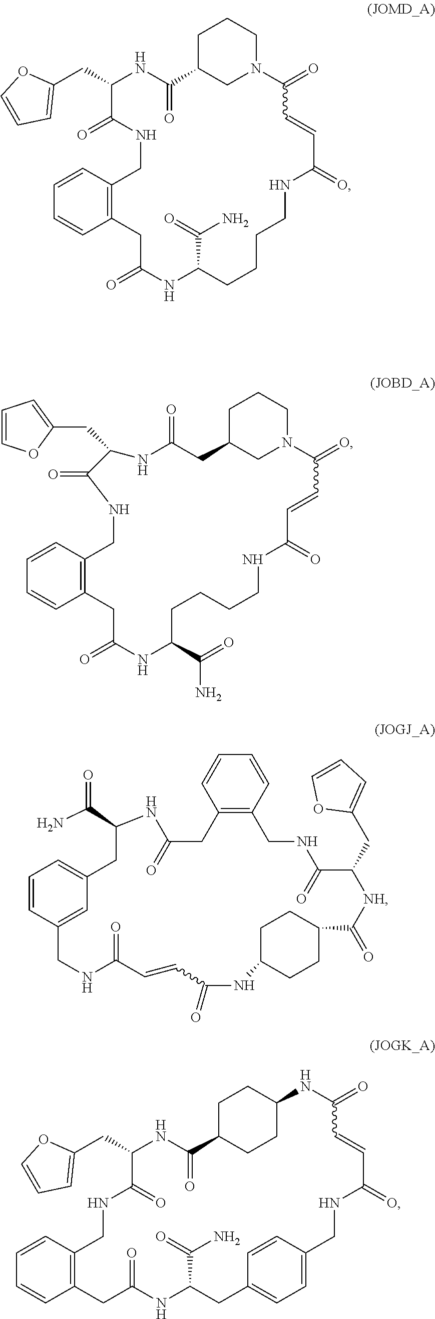 [SMILES: NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1] 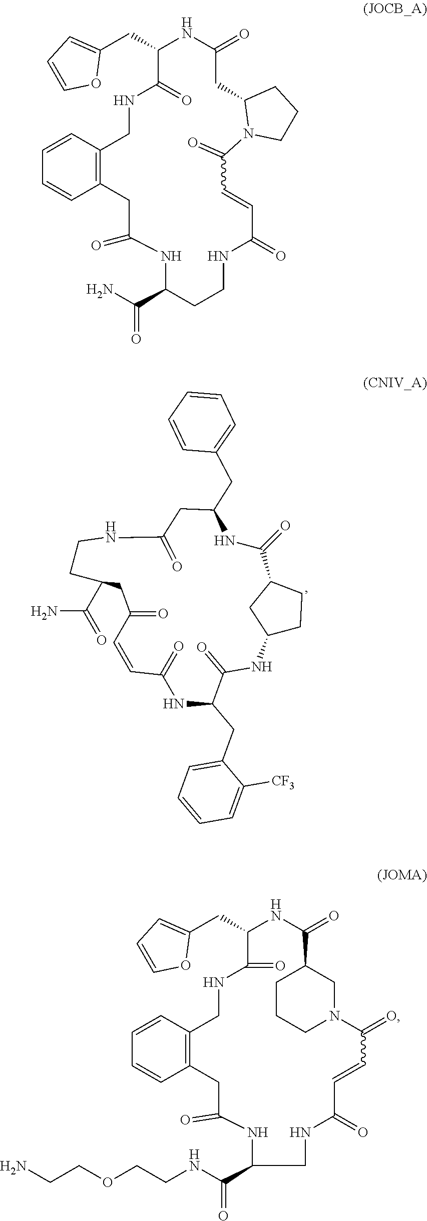 [SMILES: NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)/C=C\C(=O)C1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1] 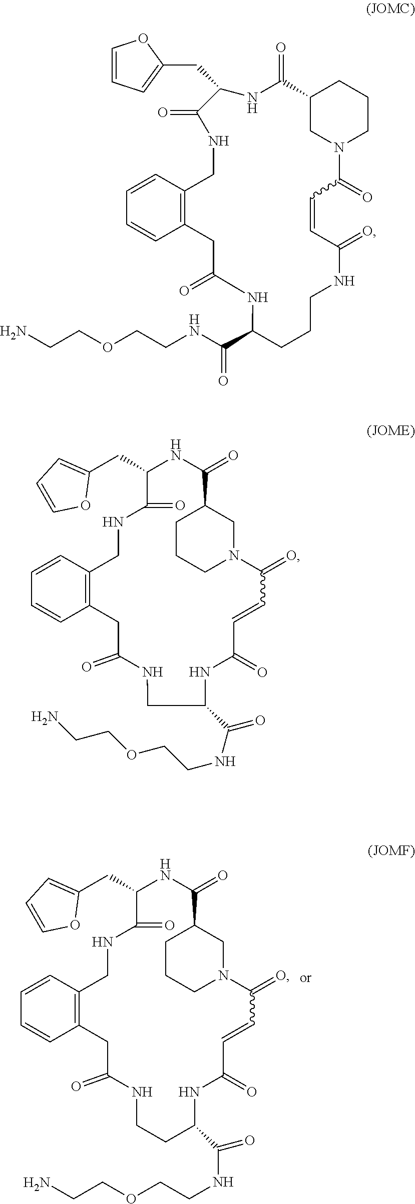 [SMILES: NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1] 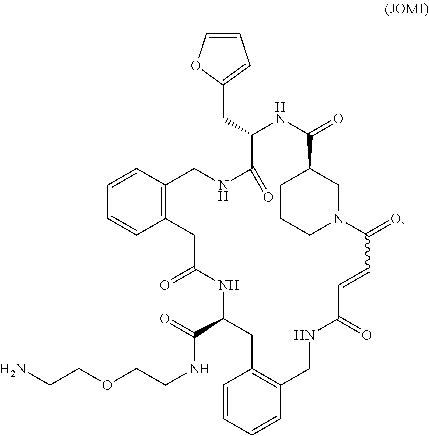 [SMILES: NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]
or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48.

65. A method of inhibiting a cyclophilin in a subject in need thereof, the method comprising:
administering to the subject a therapeutically effective amount of a compound of any one of claims 1-46, or a compound of formula: 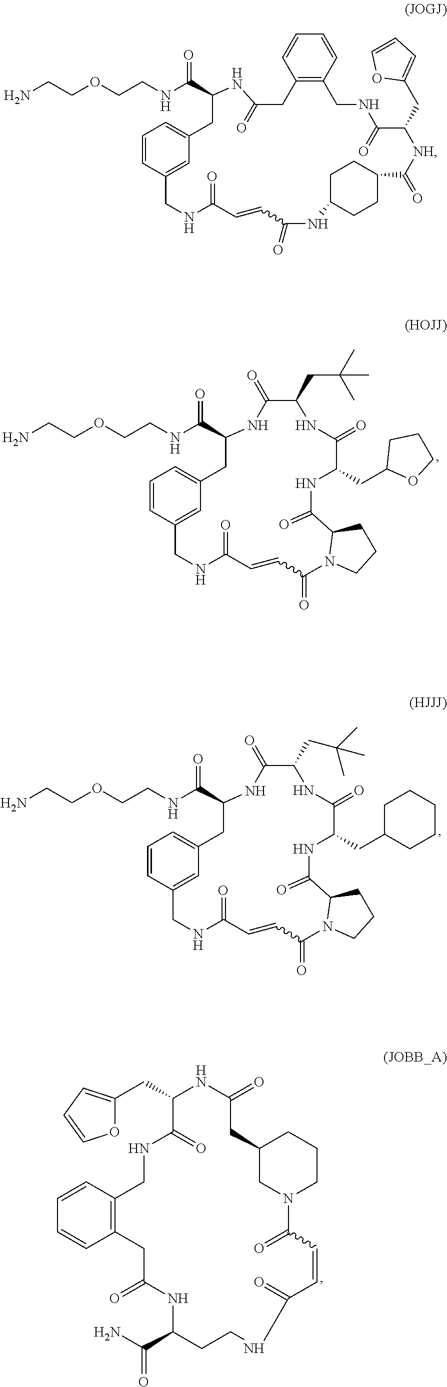 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](CC2CCCO2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2] 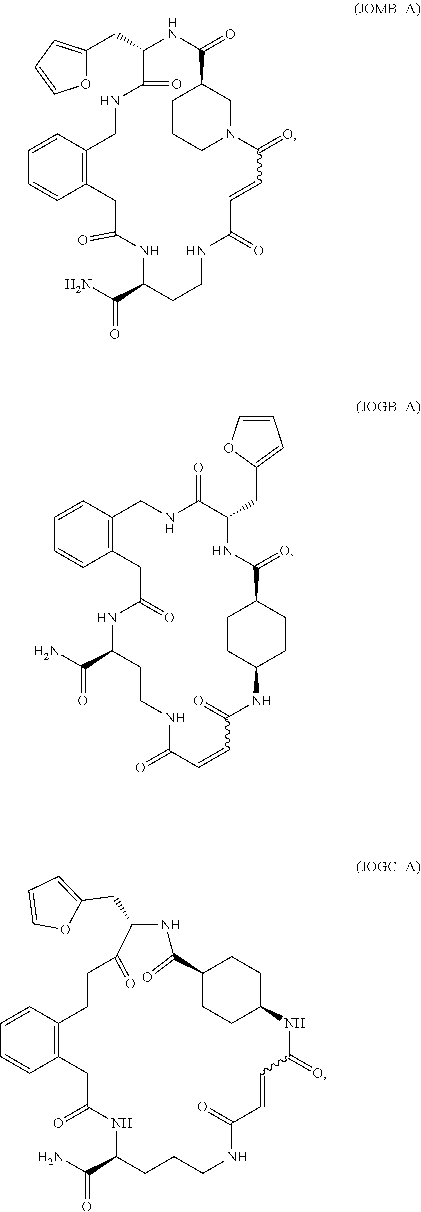 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)CCc2ccccc2CC(=O)N1] 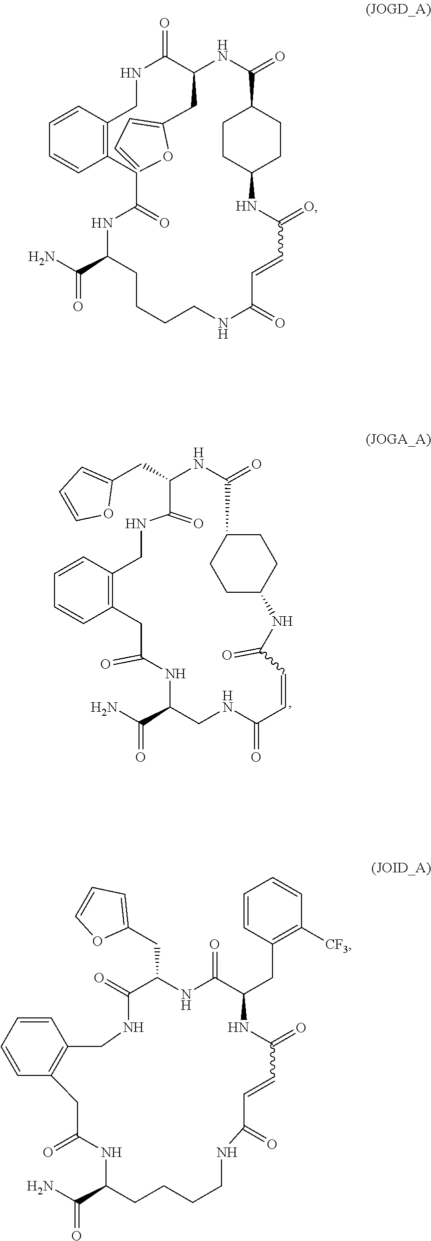 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1] 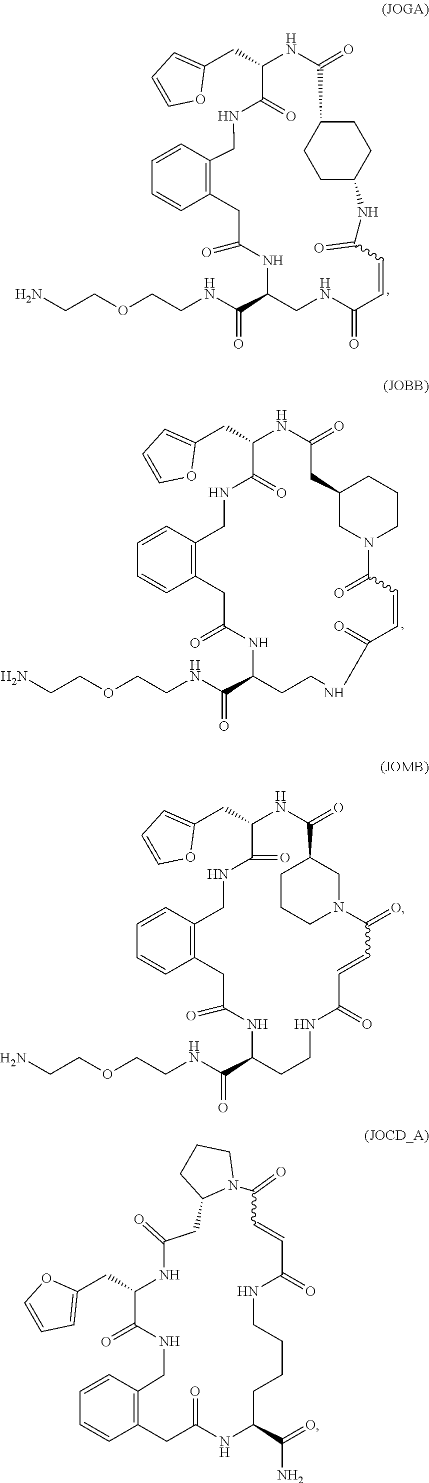 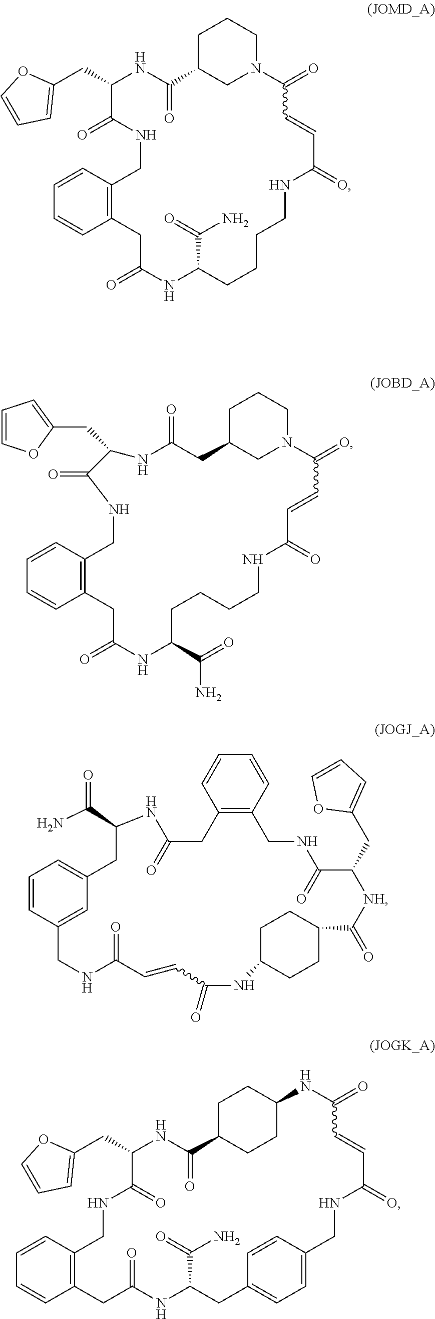 [SMILES: NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1] 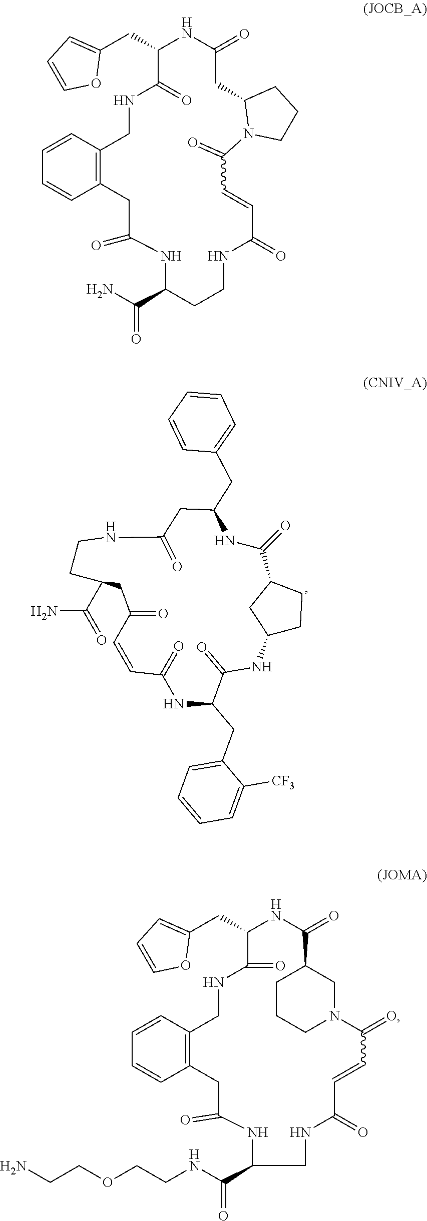 [SMILES: NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)/C=C\C(=O)C1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1] 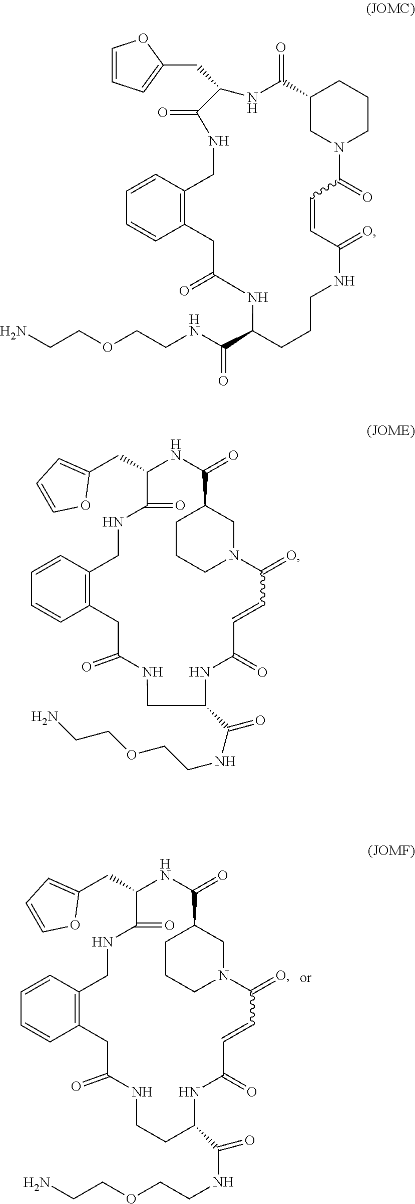 [SMILES: NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1] 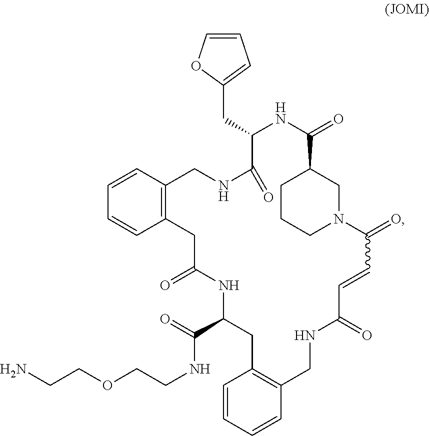 [SMILES: NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]
or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48.

66. A method of inhibiting a cyclophilin in a biological sample, the method comprising:
contacting the biological sample with an effective amount of a compound of any one of claims 1-46, or a compound of formula: 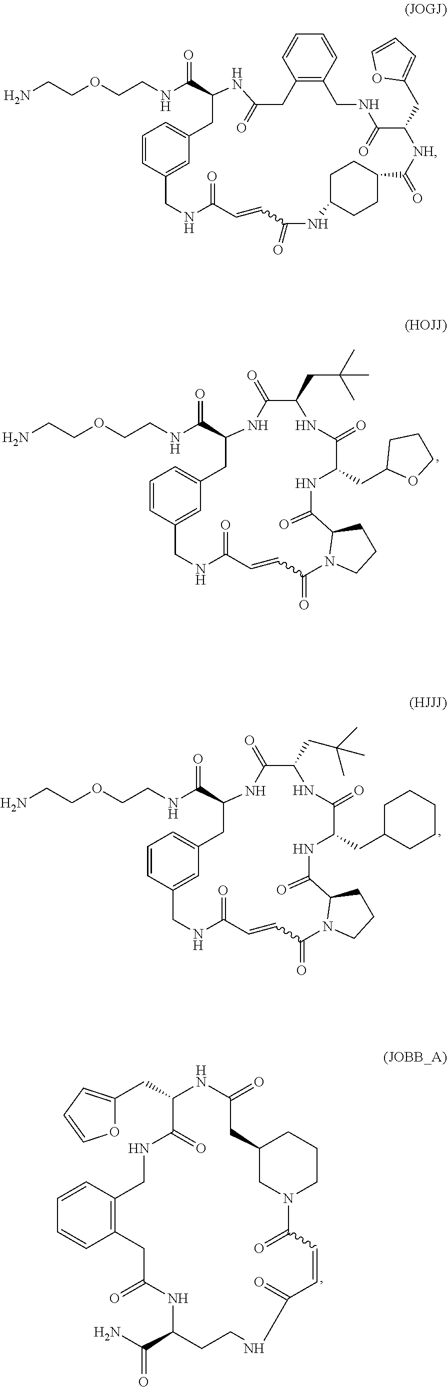 [SMILES: NCCOCCNC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; CC(C)(C)C[C@H]1NC(=O)[C@H](CC2CCCO2)NC(=O)[C@H]2CCCN2C(=O)C=CC(=O)NCc2cccc(c2)C[C@@H](C(=O)NCCOCCN)NC1=O ; CC(C)(C)C[C@@H]1NC(=O)[C@H](CC2CCCCC2)NC(=O)[C@H]2CCCN2C(=O)C=CC(=O)NCc2cccc(c2)C[C@@H](C(=O)NCCOCCN)NC1=O ; NC(=O)[C@@H]1CCNC(=O)C=CC(=O)N2CCC[C@H](CC(=O)N[C@@H](Cc3ccco3)C(=O)NCc3ccccc3CC(=O)N1)C2] 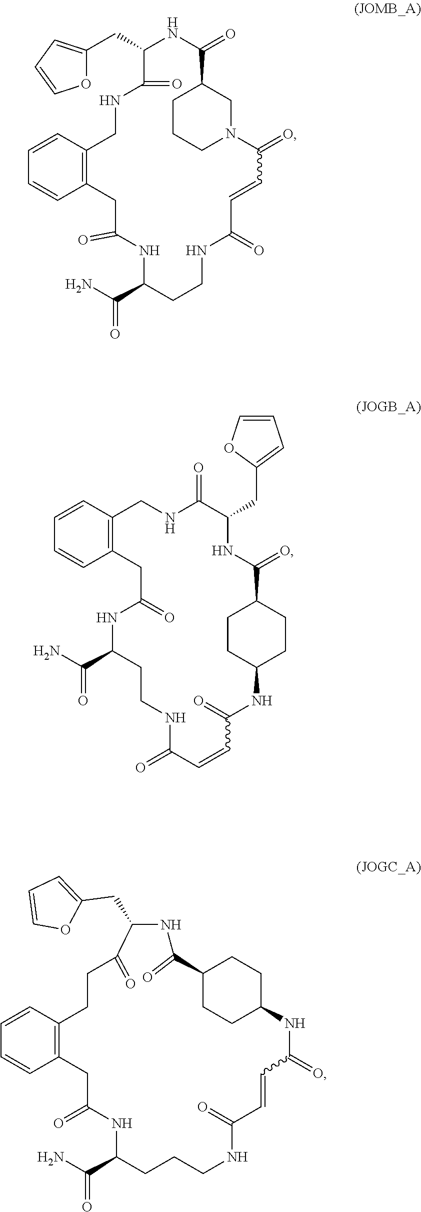 [SMILES: NC(=O)[C@@H]1CCNC(=O)C=CC(=O)N2CCC[C@H](C2)C(=O)N[C@@H](Cc2ccco2)C(=O)NCc2ccccc2CC(=O)N1 ; NC(=O)[C@@H]1CCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCNC(=O)C=CC(=O)N[C@H]2CC[C@H](CC2)C(=O)N[C@@H](Cc2ccco2)C(=O)CCc2ccccc2CC(=O)N1] 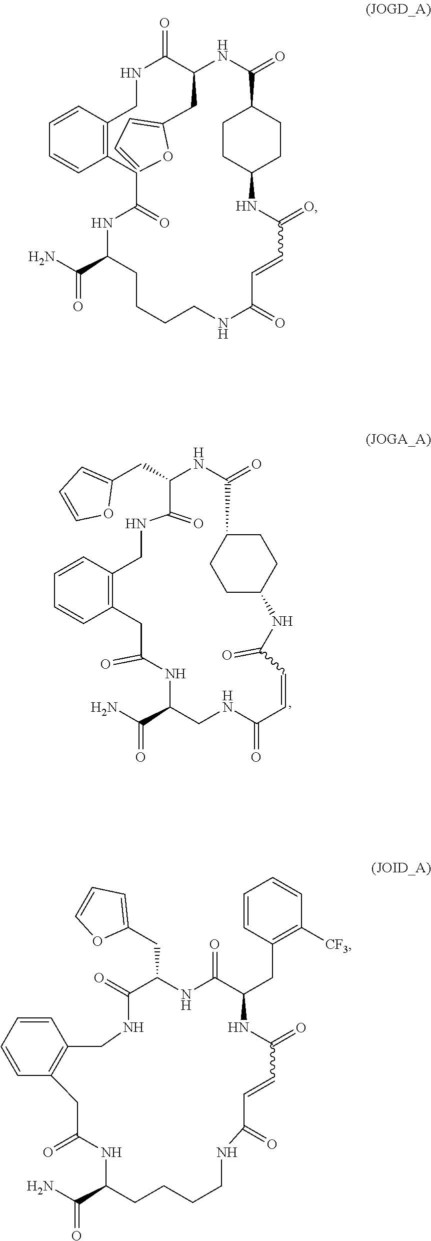 [SMILES: NC(=O)[C@@H]1CCCCNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1CCCCNC(=O)C=CC(=O)N[C@H](Cc2ccccc2C(F)(F)F)C(=O)N[C@@H](Cc2ccco2)C(=O)NCc2ccccc2CC(=O)N1] 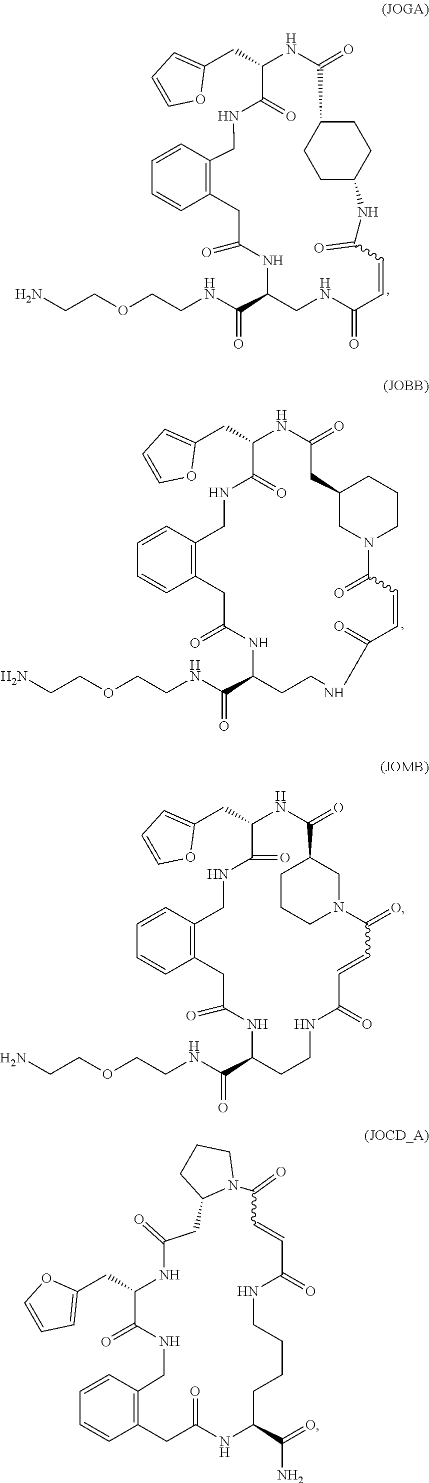 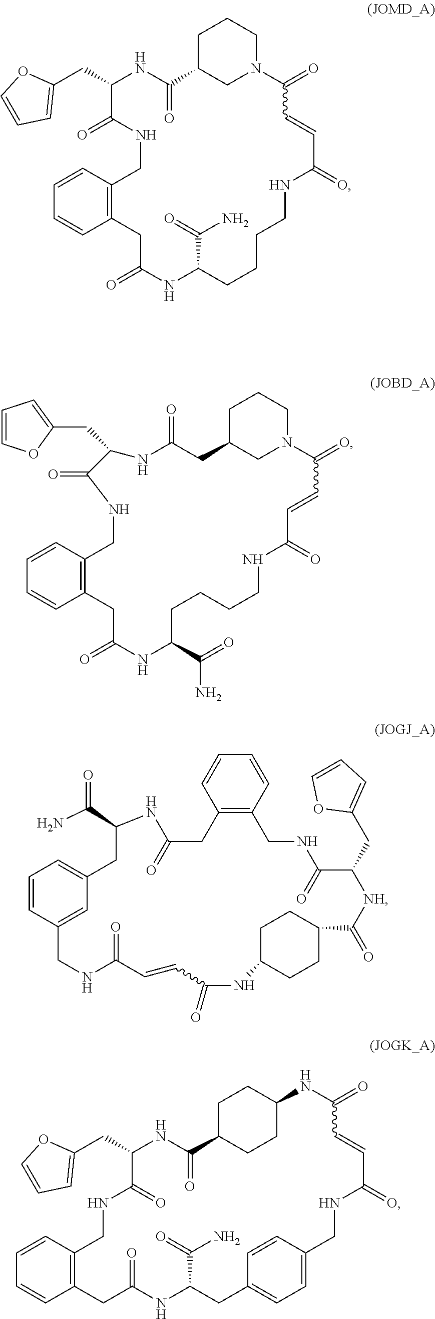 [SMILES: NC(=O)[C@@H]1CCCCNC(=O)C=CC(=O)N2CCC[C@H](C2)C(=O)N[C@@H](Cc2ccco2)C(=O)NCc2ccccc2CC(=O)N1 ; NC(=O)[C@@H]1CCCCNC(=O)C=CC(=O)N2CCC[C@H](CC(=O)N[C@@H](Cc3ccco3)C(=O)NCc3ccccc3CC(=O)N1)C2 ; NC(=O)[C@@H]1Cc2cccc(c2)CNC(=O)C=CC(=O)N[C@H]2CC[C@H](CC2)C(=O)N[C@@H](Cc2ccco2)C(=O)NCc2ccccc2CC(=O)N1 ; NC(=O)[C@@H]1Cc2ccc(cc2)CNC(=O)C=CC(=O)N[C@H]2CC[C@H](CC2)C(=O)N[C@@H](Cc2ccco2)C(=O)NCc2ccccc2CC(=O)N1] 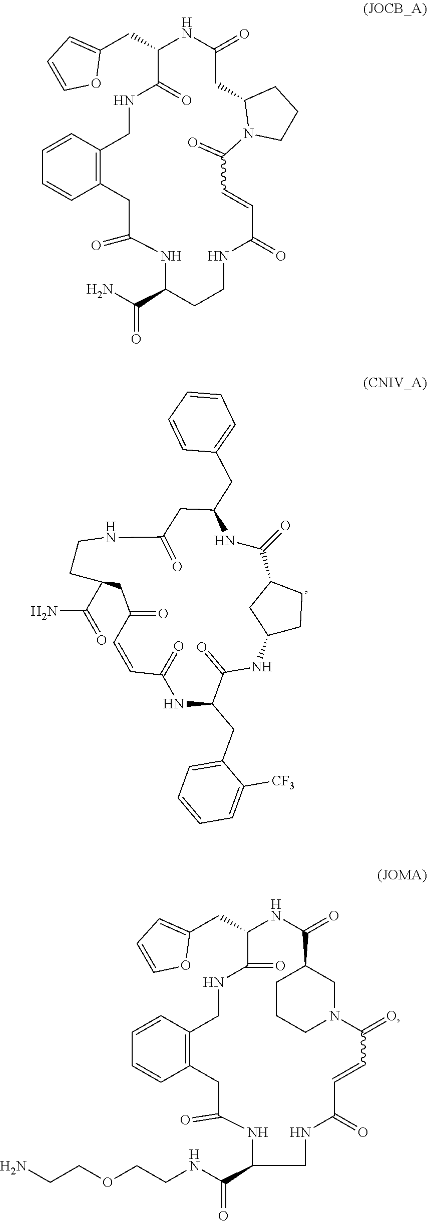 [SMILES: NC(=O)[C@@H]1CCNC(=O)C=CC(=O)N2CCC[C@H]2CC(=O)N[C@@H](Cc2ccco2)C(=O)NCc2ccccc2CC(=O)N1 ; NC(=O)[C@H]1CCNC(=O)C[C@@H](Cc2ccccc2)NC(=O)[C@@H]2CC[C@@H](C2)NC(=O)[C@@H](Cc2ccccc2C(F)(F)F)NC(=O)/C=C\C(=O)C1 ; NCCOCCNC(=O)[C@@H]1CNC(=O)C=CC(=O)N2CCC[C@H](C2)C(=O)N[C@@H](Cc2ccco2)C(=O)NCc2ccccc2CC(=O)N1] 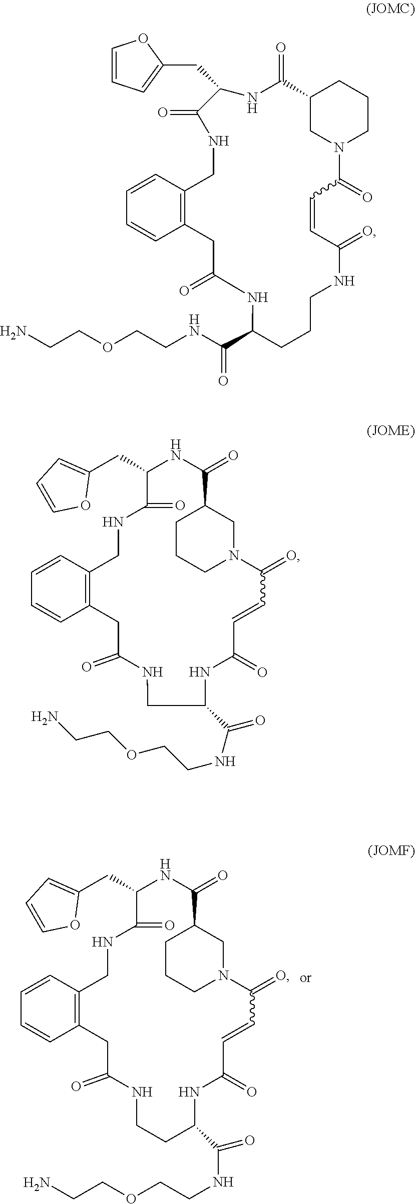 [SMILES: NCCOCCNC(=O)[C@@H]1CCCNC(=O)C=CC(=O)N2CCC[C@H](C2)C(=O)N[C@@H](Cc2ccco2)C(=O)NCc2ccccc2CC(=O)N1 ; NCCOCCNC(=O)[C@@H]1CNC(=O)Cc2ccccc2CNC(=O)[C@H](Cc2ccco2)NC(=O)[C@@H]2CCCN(C2)C(=O)C=CC(=O)N1 ; NCCOCCNC(=O)[C@@H]1CCNC(=O)Cc2ccccc2CNC(=O)[C@H](Cc2ccco2)NC(=O)[C@@H]2CCCN(C2)C(=O)C=CC(=O)N1] 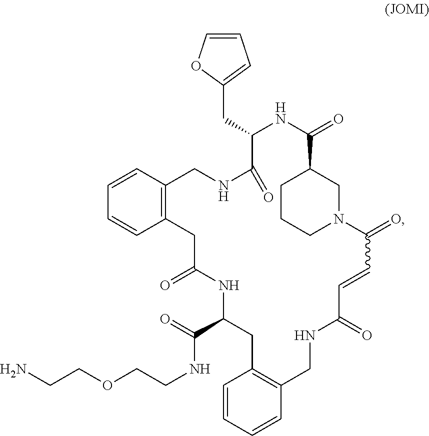 [SMILES: NCCOCCNC(=O)[C@@H]1Cc2ccccc2CNC(=O)C=CC(=O)N2CCC[C@H](C2)C(=O)N[C@@H](Cc2ccco2)C(=O)NCc2ccccc2CC(=O)N1]
or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48.

67. The method of claim 65 or 66, wherein the cyclophilin is cyclophilin A.

68. The method of claim 65 or 66, wherein the cyclophilin is cyclophilin B.

69. The method of claim 65 or 66, wherein the cyclophilin is cyclophilin C.

70. The method of claim 65 or 66, wherein the cyclophilin is cyclophilin D.

71. The method of any one of claims 65, 66, or 70, wherein the compound is capable of selectively inhibiting cyclophilin D over other cyclophilins.

72. The method of any one of claims 65, 66, 70, or 71, wherein the compound is capable of selectively inhibiting cyclophilin D over cyclophilin E.

73. The method of any one of claims 65, 66, 70, or 71, wherein the compound is capable of selectively inhibiting cyclophilin D over cyclophilin B.

74. The method of any one of claims 65, 66, 70, or 71-73, wherein the compound is capable of selectively inhibiting cyclophilin D over cyclophilins B and E.

75. The method of any one of claims 65, 66, or 70-74, wherein the compound is capable of binding the S2 pocket of cyclophilin D.

76. The method of any one of claims 65, 66, or 70-75, wherein the compound is capable of binding gatekeeper residues of cyclophilin D.

77. The method of any one of claims 65, 66, or 70-76, wherein the compound is capable of binding the gatekeeper residues serine and/or arginine of cyclophilin D.

78. The method of claim 77, wherein the gatekeeper residues comprise Serine 123 and/or Arginine 124.

79. The method of claim 65 or 66, wherein the cyclophilin is cyclophilin E.

80. The method of claim 65 or 66, wherein the cyclophilin is cyclophilin G.

81. The method of claim 65 or 66, wherein the cyclophilin is cyclophilin H.

82. The method of claim 65 or 66, wherein the cyclophilin is cyclophilin 40.

83. The method of claim 65 or 66, wherein the cyclophilin is PPWD1.

84. The method of claim 65 or 66, wherein the cyclophilin is PPIL1.

85. The method of claim 65 or 66, wherein the cyclophilin is NKTR.

86. The method of any one of claims 49-63, 65, or 67-82 further comprising administering to the subject a therapeutically effective amount of an additional pharmaceutical agent in combination with the compound, or a pharmaceutically acceptable salt, solvate, hydrate, tautomer, or stereoisomer thereof, or the pharmaceutical composition of any one of claims 47 or 48.

87. The method of any one of claims 64 or 66-82 further comprising contacting the biological sample with a therapeutically effective amount of an additional pharmaceutical agent in combination with the compound, or a pharmaceutically acceptable salt, solvate, hydrate, tautomer, or stereoisomer thereof, or the pharmaceutical composition of any one of claims 47 or 48.

88. The method of claim 83 or 84, wherein the additional pharmaceutical agent is an agent for treating a disease associated with cyclophilins.

89. Use of a compound of any one of claims 1-46, or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48 to treat a disease and/or condition in a subject in need thereof.

90. A kit comprising:
a compound of any one of claims 1-46, or a pharmaceutically acceptable salt, solvate, hydrate, polymorph, co-crystal, tautomer, stereoisomer, isotopically labeled derivative, prodrug, composition, or mixture thereof, or a pharmaceutical composition of claim 47 or 48; and
instructions for administering to a subject or contacting a biological sample with the compound, or the pharmaceutically acceptable salt, solvate, hydrate, polymorph, co-crystal, tautomer, stereoisomer, isotopically labeled derivative, prodrug, composition, or mixture thereof, or the pharmaceutical composition of claim 47 or 48.
